(19) United States
(12) Patent Application Publication
(10) Pub. No.: US 2023/0406843 A1
(43) Pub. Date: Dec. 21, 2023

(54) Progranulin Modulators and Methods of Using the Same

(71) Applicant: ARKUDA THERAPEUTICS, Watertown, MA (US)

(72) Inventors: Duane A. Burnett, Watertown, MA (US); James C. Lanter, Watertown, MA (US)

(21) Appl. No.: 18/038,749

(22) PCT Filed: Dec. 10, 2021
(86) PCT No.: PCT/US21/62733

§ 371 Date: May 25, 2023

Related U.S. Application Data
(60) Provisional application No. 63/123,651, filed on Dec. 10, 2020.

(51) Int. Cl. C07D 405/06; C07D 498/04; C07D 491/107; C07D 413/06; C07D 491/052; C07D 498/10; C07D 513/04; C07D 405/14; A61P 25/28

(52) U.S. Cl. CPC C07D 405/06; C07D 498/04; C07D 491/107; C07D 413/06; C07D 491/052; C07D 498/10; C07D 513/04; C07D 405/14; A61P 25/28

(57) ABSTRACT

Provided herein are compounds that modulate progranulin and methods of using the compounds in progranulin-associated disorders, such as Frontotemporal lobe dementia (FTLD).

### BACKGROUND

Provided herein are compounds that modulate progranulin levels and can be useful as therapeutics for granulin (GRN)- and/or progranulin (PGRN)-associated disorders. Mutations in the GRN gene cause Frontotemporal lobar degeneration (FTLD) (see, e.g., Cruts et al., Granulin Mutations Associated with Frontotemporal Lobar Degeneration and Related Disorders: An Update, Hu Mutation, 2008 and Baker et al., Nature, 2006.) FTLD-associated mutations in GRN result in a reduction of progranulin protein expression, which suggests that haploinsufficiency of progranulin is the critical pathogenic factor in FTLD-GRN. Plasma and CSF progranulin levels are reduced by up to 70% in pathogenic GRN mutation carriers (Ghidoni, et al., Neurodegen Dis, 2012). More than 60 non-sense mutations in the GRN gene have been described. Plasma can be easily monitored for PGRN (see e.g., Meeter, Nature Neurology, volume 13, 2017). Thus, granulin- and/or progranulin-associated disorders can be modulated by compounds which increase progranulin secretion and/or activity.

All known FTLD-GRN-associated mutations cause haploinsufficiency of progranulin, suggesting that restoration of proper progranulin levels or progranulin protein function will be therapeutically beneficial for FTLD-GRN patients. Several studies have shown that even subtle reductions in progranulin levels by genetic modifiers (e.g., TMEM106B, SLPI, Rs5848) have significant effects on the age-of-onset of FTLD, increase the risk of developing FTLD, or worsen the course of autoimmune diseases such as osteoarthritis (see, e.g., Nicholson et al., J Neurochem, 2013; Cruchaga et al., Arch Neurol, 2012; and Wei et al, Plos One, 2014). Polymorphisms that affect progranulin levels have also been identified as genetic modifiers of several other neurodegenerative diseases, such as Alzheimer's disease and C9orf72-linked FTLD (see, e.g., Sheng et al., Gene, 2014 and van Blitterswijk et al., Mol Neurodegen, 2014). As such, it is contemplated herein that progranulin-targeted therapeutics are effective across multiple neurodegenerative and autoimmune disorders.

Granulins are a family of secreted and glycosylated proteins. They are cleaved from a common precursor protein called progranulin (PGRN). Progranulin is a secreted glycoprotein and is expressed in neurons, neuroglia, chondrocytes, epithelial cells and leukocytes (Toh H et al. J Mol Neurosci 201 1 November; 45(3):538-48). It is a precursor protein with an N-terminal signal peptide and seven granulin motifs. Each of these granulin motifs contains 12 cysteines, which are responsible for 6 disulfide bridges in every granulin (Bateman A et al. Bioessays 2009:1245-54). Progranulin is coded by the GRN gene. Mutations in the GRN gene have been implicated in up to 25% of frontotemporal lobar degeneration, inherited in an autosomal dominant fashion with high penetrance (see, e.g., Mackenzie, Acta Neuropathologica, 114(1): 49-54 (2007)). Thus, modulation of progranulin activity is an attractive target for treating disorders associated with GRN activity or GRN-gene mutations.

The translocon complex is the main gate to the secretory pathway. It facilitates the translocation of nascent proteins into the endoplasmic reticulum (ER) lumen or their integration in lipid membranes. The translocon is organized around a conserved core composed of a trimeric protein complex, the Sec61 channel. It is associated with cytosolic chaperones such as the signal recognition particle (SRP), auxiliary components such as translocating chain-associating membrane (TRAM), translocon-associated protein (TRAP) and modifying enzymes such as oligosaccharyltransferase (OST). The latter is responsible for proper glycosylation of proteins and binds to the ribosome-Sec61-TRAP complex in near stoichiometric ratio. The importance of this complex is highlighted by mutations found in one of the TRAP components of the complex that lead to glycosylation disorders.

The lysosome is an organelle containing over 60 different enzymes such as lipases, proteases and hydrolases that are mainly involved in the breakdown of proteins, lipids and carbohydrates. Mutations found in multiple lysosomal proteins are the underlying cause of multiple different disease grouped under the classification of neuronal ceroid lipofuscinosis (NCL) also known as Batten disease. In some cases, certain lysosomal proteins act as co-factors of lysosomal enzymes. Their distribution and/or level of expression can modulate the activity of the lysosomal enzymes and as such, have regulatory downstream function on the overall function of the lysosome.

### SUMMARY

Provided herein are compounds and methods for modulating progranulin, e.g., increasing the level of progranulin or granulin in a subject. More particularly, provided are modulators of progranulin and the uses of such modulators in treating progranulin-associated disorders, e.g., Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), lysosomal storage diseases, diseases associated with inclusions and/or misfunction of C9orf72, TDP-43, FUS, UBQLN2, VCP, CHMP28, and/or MAPT, acute neurological disorders, glioblastoma, or neuroblastoma.

The disclosure provides compounds of Formula (I):

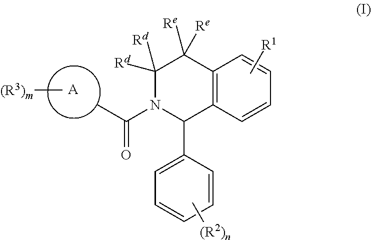

wherein ring A is a 4- to 12-membered heterocycle comprising a ring O or S atom, further comprising 0-3 additional ring heteroatoms selected from O, N, and S; R¹ is hydrogen, C₁₋₆alkyl, halo, C₁₋₃ haloalkyl, O—C₁₋₃ haloalkyl,C₁₋₃ alkylene-CN, C₁₋₃ alkylene-NR^{N}₂, C₀₋₆ alkylene-OR^{N}, C₀₋₆ alkylene- C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2; each R² is independently halo; each R³ is independently hydrogen, halo, C₁₋₆alkyl, C₁₋₆haloalkyl, C₀₋₆alkylene-OH, C₁₋₆ alkoxy, C₁₋₆haloalkoxy, C₁₋₆alkylene-O—C₁₋₆alkyl, C₀₋₆alkylene-NR^{a}R^{b}, S—C₁₋₆alkyl, C₂₋₆alkenyl, C(O)—C₁₋₆haloalkyl, SO₂—C₁₋₆alkyl, S²⁺(O)—(NR^{a})—C₁₋₆alkyl, OR⁴, 5- to 8-membered heteroaryl comprising 1-4 ring N atoms, or 4- to 6-membered heterocycle comprising 1-4 ring heteroatoms selected from O, N, and S with at least 1 ring heteroatom being N, and the heteroaryl or heterocycle is optionally substituted with 1 or 2 substituents independently selected from halo, C₁₋₆alkyl, OH, and C₁₋₆alkoxy, or two geminal R³ together with the atom to which they are attached form an oxo group, and when ring A comprises a ring N atom, the N is substituted with R^{a}, and if ring A does not comprise a ring N atom, then at least one R³ is C₀₋₆alkylene-NR^{a}R^{b}; R^{a} and R^{b} are each independently hydrogen, C₁₋₆alkyl, C₁₋₆haloalkyl, C₁₋₆alkylene-OH, C₁₋₆alkylene-O—C₁₋₆alkyl, C(O)—C₁₋₆alkyl, C(O)—C₁₋₆haloalkyl, S(O)₂—C₁₋₆alkyl, S(O)₂—C₁₋₆haloalkyl; or R^{a} and R^{b} together with the nitrogen to which they are attached form a 3- to 12-membered monocyclic or bicyclic heterocycle optionally further comprising 1-3 additional ring heteroatoms selected from O, N, and S; R⁴ is C₂₋₆alkenyl, C₂₋₆alkynyl, C₀₋₆alkylene-C₃₋₆cycloalkyl, or C₀₋₆alkylene-C₆₋₁₀aryl; each R^{d} is independently H or D; each R^{e} is independently H, D, halo, OH, methyl, methoxy, or two geminal R^{e} together with the atom to which they are attached form an oxo group or a spiro C₃₋₅ cycloalkyl; m is 1-4; and n is 0-2.

Also provided are compounds of Formula (Ia):

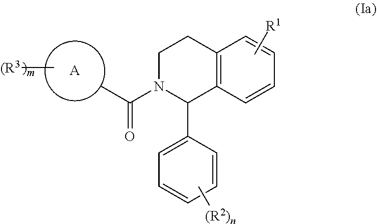

wherein A is a 4- to 12-membered heterocycle comprising a ring O or S atom, further comprising 0-3 additional ring heteroatoms selected from O, N, and S; R¹ is hydrogen, C₁₋₆alkyl, halo, C₁₋₆haloalkyl, O—C₁₋₆ haloalkyl, C₀₋₃ alkylene-CN, C₀₋₃ alkylene-NR^{N}₂, C₀₋₆alkylene-OR^{N}, C₀₋₆ alkylene- C(O)OR^{N}, C₀₋₆alkylene-C(O)N(R^{N})₂, or C₀₋₆ alkylene-SOₚR^{N}; each R^{N} is independently hydrogen or C₁₋₆alkyl, and p is 0-2; each R² is independently halo; each R³ is independently hydrogen, halo, C₁₋₆alkyl, C₁₋₆haloalkyl, C₀₋₆alkylene-OH, C₁₋₆alkoxy, C₁₋₆haloalkoxy, C₁₋₆ alkylene-O—C₁₋₆alkyl, C₀₋₆alkylene-NR^{a}R^{b}, S—C₁₋₆alkyl, C₂₋₆alkenyl, C(O)—C₁₋₆haloalkyl, or SO₂—C₁₋₆alkyl, or two geminal R³ together with the atom to which they are attached form an oxo group, and when ring A comprises a ring N atom, the N is substituted with Rₐ, and if ring A does not comprise a ring N atom, then at least one R³ is C₀₋₆alkylene-NR^{a}R^{b}; R^{a} and R^{b} are each independently hydrogen, C₁₋₆alkyl, C₁₋₆alkylene-OH, C₁₋₆alkylene-O—C₁₋₆ alkyl, C(O)—C₁₋₆alkyl, C(O)—C₁₋₆haloalkyl, S(O)₂—C₁₋₆alkyl, S(O)₂—C₁₋₆haloalkyl; or R^{a} and R^{b} together with the nitrogen to which they are attached form a 5- to 12-membered monocyclic or bicyclic heterocycle optionally further comprising 1-3 additional ring heteroatoms selected from O, N, and S; m is 1-3; and n is 0-2.

In some cases, ring A is

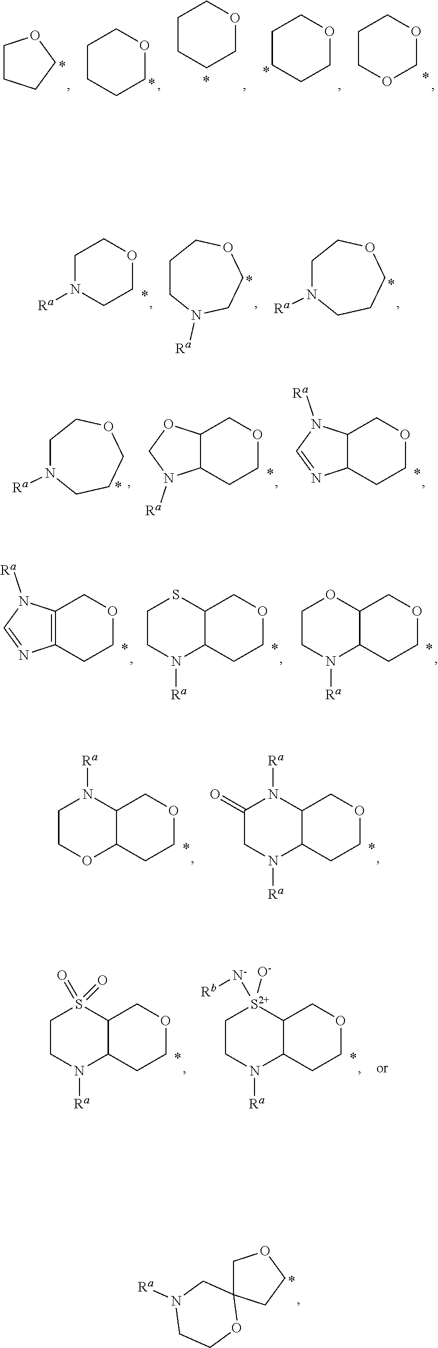

wherein * indicates the point of attachment of ring A to the adjacent carbonyl moiety of Formula I. In some cases, ring A is,

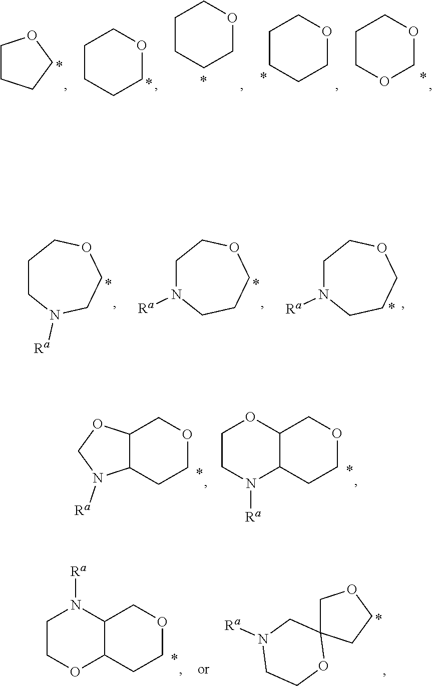

wherein * indicates the point of attachment of ring A to the adjacent carbonyl moiety of Formula I. In some cases, ring A is.

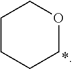

Further provided are methods of modulating progranulin in a subject. In some embodiments, provided are methods of treating a progranulin-associated disorder in a subject.

Other aspects of the disclosure include a compound as disclosed herein for use in the preparation of a medicament for the modulation of progranulin, and the use of a compound as disclosed herein in a method of treating or preventing a progranulin-associated disorder in a subject.

### DETAILED DESCRIPTION

### Compounds as Progranulin Modulators

Provided herein are compounds that can modulate progranulin production and/or secretion. In some cases, the compounds can increase the level of progranulin or granulin in a subject. Also provided are methods of modulating the translocon complex using compounds as disclosed herein to increase lysosomal protein levels.

The disclosure provides compounds of Formula (I):

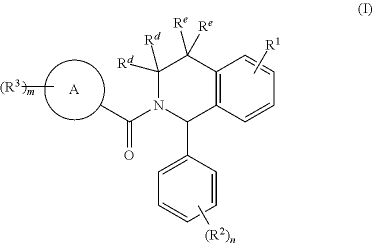

wherein
ring A is a 4- to 12-membered heterocycle comprising a ring O or S atom, further comprising 0-3 additional ring heteroatoms selected from O, N, and S;
R¹ is hydrogen, C₁₋₆alkyl, halo, C₁₋₃ haloalkyl, O—C₁₋₃ haloalkyl,C₁₋₃ alkylene-CN, C₁₋₃ alkylene-NR^{N}₂, C₀₋₆ alkylene-OR^{N}, C₀₋₆ alkylene- C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
each R² is independently halo;
each R³ is independently hydrogen, halo, C₁₋₆alkyl, C₁₋₆haloalkyl, C₀₋₆alkylene-OH, C₁₋₆alkoxy, C₁₋₆ haloalkoxy, C₁₋₆alkylene-O—C₁₋₆alkyl, C₀₋₆alkylene-NR^{a}R^{b}, S—C₁₋₆alkyl, C₂₋₆alkenyl, C(O)—C₁₋₆haloalkyl, SO₂—C₁₋₆ alkyl, S²⁺(O)—(NR^{a})—C₁₋₆alkyl, OR⁴, 5- to 8-membered heteroaryl comprising 1-4 ring N atoms, or 4- to 6-membered heterocycle comprising 1-4 ring heteroatoms selected from O, N, and S with at least 1 ring heteroatom being N, and the heteroaryl or heterocycle is optionally substituted with 1 or 2 substituents independently selected from halo, C₁₋₆alkyl, OH, and C₁₋₆alkoxy, or
two geminal R³ together with the atom to which they are attached form an oxo group, and
when ring A comprises a ring N atom, the N is substituted with R^{a}, and if ring A does not comprise a ring N atom, then at least one R³ is C₀₋₆alkylene-NR^{a}R^{b};
R^{a} and R^{b} are each independently hydrogen, C₁₋₆alkyl, C₁₋₆haloalkyl, C₁₋₆alkylene-OH, C₁₋₆alkylene-O—C₁₋₆alkyl, C(O)—C₁₋₆alkyl, C(O)—C₁₋₆haloalkyl, S(O)₂—C₁₋₆alkyl, S(O)₂—C₁₋₆haloalkyl; or
R^{a} and R^{b} together with the nitrogen to which they are attached form a 3- to 12-membered monocyclic or bicyclic heterocycle optionally further comprising 1-3 additional ring heteroatoms selected from O, N, and S;
R⁴ is C₂₋₆alkenyl, C₂₋₆alkynyl, C₀₋₆alkylene-C₃₋₆cycloalkyl, or C₀₋₆alkylene-C₆₋₁₀aryl;
each R^{d} is independently H or D;
each R^{e} is independently H, D, halo, OH, methyl, or methoxy, or
two geminal R^{e} together with the atom to which they are attached form an oxo group or a spiro C₃₋₅cycloalkyl;
m is 1-4; and
n is 0-2.

Also provided are compounds of Formula (Ia):

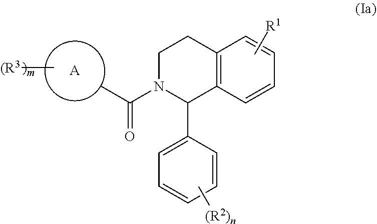

wherein
A is a 4- to 12-membered heterocycle comprising a ring O or S atom, further comprising 0-3 additional ring heteroatoms selected from O, N, and S;
R¹ is hydrogen, C₁₋₆alkyl, halo, C₁₋₃ haloalkylene, O—C₁₋₃ haloalkylene, C₁₋₃ alkylene-CN, C₁₋₃ alkylene-NR^{N}₂, C₀₋₆ alkylene-OR^{N}, C₀₋₆ alkylene- C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
each R² is independently halo;
each R³ is independently hydrogen, halo, C₁₋₆alkyl, C₁₋₆haloalkyl, C₀₋₆alkylene-OH, C₁₋₆alkoxy, C₁₋₆ haloalkoxy, C₁₋₆alkylene-O—C₁₋₆alkyl, C₀₋₆alkylene-NR^{a}R^{b}, S—C₁₋₆alkyl, C₂₋₆alkenyl, C(O)—C₁₋₆haloalkyl, or SO₂—C₁₋₆ alkyl, or
two geminal R³ together with the atom to which they are attached form an oxo group, and when ring A comprises a ring N atom, the N is substituted with Rₐ, and if ring A does not comprise a ring N atom, then at least one R³ is C₀₋₆alkylene-NR^{a}R^{b};
R^{a} and R^{b} are each independently hydrogen, C₁₋₆alkyl, C₁₋₆alkylene-OH, C₁₋₆alkylene-O—C₁₋₆alkyl, C(O)—C₁₋₆alkyl, C(O)—C₁₋₆haloalkyl, S(O)₂—C₁₋₆alkyl, S(O)₂—C₁₋₆haloalkyl; or
R^{a} and R^{b} together with the nitrogen to which they are attached form a 5- to 12-membered monocyclic or bicyclic heterocycle optionally further comprising 1-3 additional ring heteroatoms selected from O, N, and S;
m is 1-3; and
n is 0-2.

In some cases, ring A is a 4- to 6-membered heterocycle. In some cases, ring A is a 6- to 8-membered heterocycle. In some cases, ring A is a 4-membered heterocycle. In some cases, ring A is a 5-membered heterocycle. In some cases, ring A is a 6-membered heterocycle. In some cases, ring A is a 7-membered heterocycle. In some cases, ring A is an 8-membered heterocycle. In some cases, ring A is a 9-membered heterocycle. In some cases, ring A is a 10-membered heterocycle. In some cases, ring A is a 11-membered heterocycle. In some cases, ring A is a 12-membered heterocycle.

In some cases, ring A comprises a ring O atom and 0-3 additional ring heteroatoms selected from 0, N, and S. In some cases, ring A comprises a ring O atom and 0 additional ring heteroatoms. In some cases, ring A comprises a tetrahydropyranyl ring. In some cases, ring A comprises a ring O atom and 1 additional ring heteroatom selected from O, N, and S. In some cases, ring A comprises a ring O atom and a ring N atom. In some cases, ring A comprises a ring O atom and 2 additional ring heteroatoms selected from O, N, and S. In some cases, ring A comprises a ring O atom and 3 additional ring heteroatoms selected from O, N, and S.

In some cases, ring A comprises a ring S atom and 0-3 additional ring heteroatoms. In some cases, ring A comprises a ring S atom and 0 additional ring heteroatoms selected from O, N, and S. In some cases, ring A comprises a ring S atom and 1 additional ring heteroatom selected from O, N, and S. In some cases, ring A comprises a ring S atom and 2 additional ring heteroatoms selected from O, N, and S. In some cases, ring A comprises a ring S atom and 3 additional ring heteroatoms selected from O, N, and S.

In some cases, ring A comprises a ring N atom and the N is substituted with Rₐ. In cases where ring A comprises a ring N atom, ring A can be substituted an R³ that is C₀₋₆alkylene-NR^{a}R^{b}. In cases where ring A does not comprise a ring N atom, at least one R³ is C₀₋₆alkylene-NR^{a}R^{b}.

In some cases, ring A is

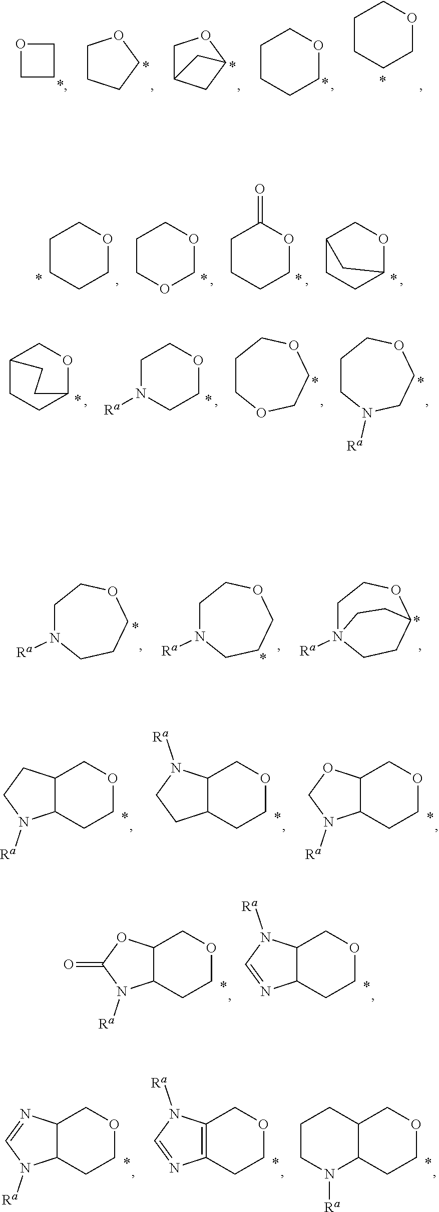

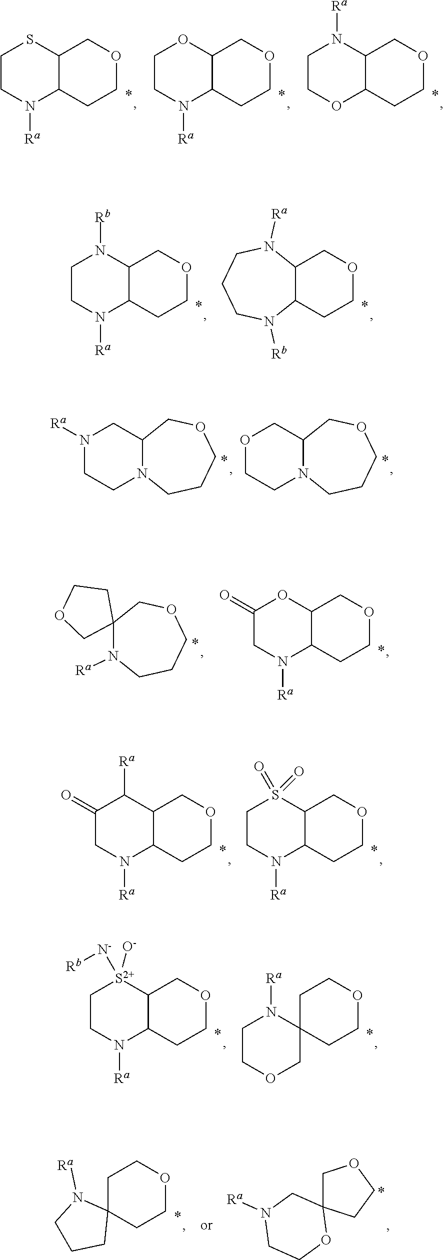

wherein * indicates the point of attachment of ring A to the adjacent carbonyl moiety of Formula I. In some cases ring A is

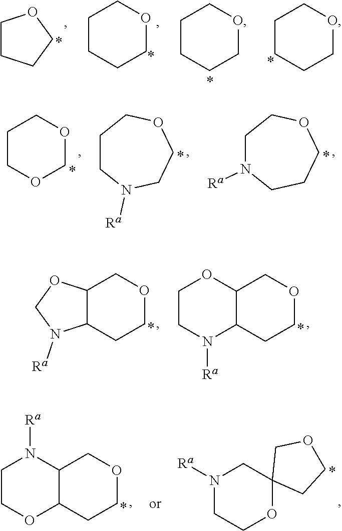

wherein * indicates the point of attachment of ring A to the adjacent carbonyl moiety of Formula I. In some cases, ring A is

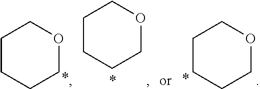

In some cases, ring A is

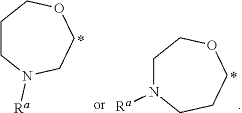

In some cases, ring A is

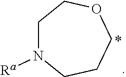

In some cases, ring A is

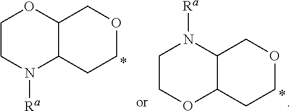

In some cases, ring A is

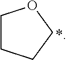

In some cases, ring A is

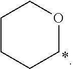

In some cases, ring A is

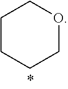

In some cases, ring A is

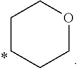

In some cases, ring A is

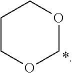

In some cases, ring A is

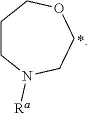

In some cases, ring A is

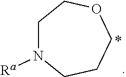

In some cases, ring A is

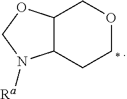

In some cases, ring A is

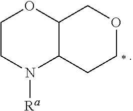

In some cases, ring A is

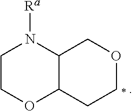

In some cases, ring A is

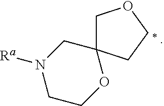

In some cases where there is a NR^{a} in ring A, the R^{a} is H. All ring A's noted in this paragraph can be substituted with m (i.e., 1 to 3) R³ substituents as discussed herein.

In some cases, m is 1-3. In some cases, m is 1 or 2. In some cases, m is 1. In some cases, m is 2. In some cases, m is 3. In some cases m is 4.

In some cases, ring A is

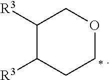

In some cases, each R³ is independently hydrogen, halo, C₁₋₆alkyl, C₁₋₆haloalkyl, C₀₋₆alkylene-OH, C₁₋₆ alkoxy, C₁₋₆haloalkoxy, C₁₋₆alkylene-O—C₁₋₆alkyl, C₀₋₆alkylene-NR^{a}R^{b}, S—C₁₋₆alkyl, C₂₋₆alkenyl, C(O)—C₁₋₆haloalkyl, or SO₂—C₁₋₆alkyl, or two geminal R³ together with the atom to which they are attached form an oxo group, and when ring A comprises a ring N atom, the N is substituted with R^{a}, and if ring A does not comprise a ring N atom, then at least one R³ is C₀₋₆alkylene-NR^{a}R^{b}.

In some cases, two geminal R³ together with the atom to which they are attached form an oxo group. In some cases, R³ is hydrogen, halo, C₁₋₆alkyl, C₁₋₆haloalkyl, C₀₋₆alkylene-OH, C₁₋₆alkoxy, C₁₋₆haloalkoxy, C₁₋₆ alkoxy-C₁₋₆alkyl, C₀₋₆alkylene-NR^{a}R^{b}, S—C₁₋₆alkyl, C₂₋₆alkene, C(O)—C₁₋₆haloalkyl, or SO₂—C₁₋₆alkyl. In some cases, R³ is hydrogen, halo, C₁₋₆alkoxy, or C₀₋₆alkylene-NR^{a}R^{b}. In some cases, R³ is halo, C₁₋₆alkoxy, or C₀₋₆alkylene-NR^{a}R^{b}. In some cases, R³ is H. In some cases, R³ is halo. In some cases, R³ is F. In some cases, R³ is C₁₋₆alkyl. In some cases, R³ is C₁₋₆haloalkyl. In some cases, R³ is C₀₋₆alkylene-OH. In some cases, R³ is C₁. 6alkoxy. In some cases, R³ is methoxy or ethoxy. In some cases, R³ is methoxy. In some cases, R³ is ethoxy. In some cases, R³ is C₁₋₆haloalkoxy. In some cases, R³ is C₁₋₆alkoxy-C₁₋₆alkyl. In some cases, R³ is C₀₋₆alkylene-NR^{a}R^{b}. In some cases, R³ is NH₂ or NHMe. In some cases, R³ is NH₂. In some cases, R³ is NHMe. In some cases, R³ is S—C₁₋₆alkyl. In some cases, R³ is C₂₋₆alkene. In some cases, C(O)—C₁₋₆haloalkyl. In some cases, R³ is SO₂—C₁₋₆alkyl.

In some cases, R³ is halo or C₀₋₆alkylene-NR^{a}R^{b}. In some cases, R³ is F or NH₂. In some cases, R³ is F or NHMe. In some cases, R³ is C₁₋₆alkoxy or C₀₋₆alkylene-NR^{a}R^{b}. In some cases, R³ is methoxy or NH₂. In some cases, R³ is methoxy or NHMe. In some cases, R³ is ethoxy or NH₂. In some cases, R³ is ethoxy or NHMe. In some cases, R³ is S²⁺(O)—(NR^{a})—C₁₋₆alkyl. In some cases, R³ is

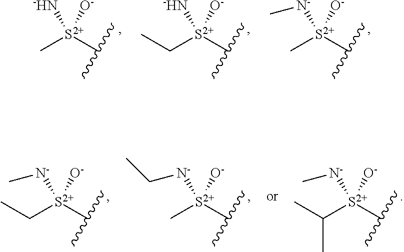

In some cases, R³ is OR⁴. In some cases, R³ is

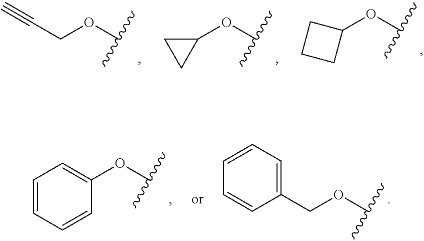

In some cases, R³ is

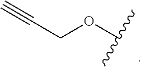 [SMILES: *OCC#C]

In some cases, R^{a} and R^{b} are each independently hydrogen, C₁₋₆alkyl, C₁₋₆alkylene-OH, C₁₋₆alkylene -O-C₁₋₆alkyl, C(O)—C₁₋₆alkyl, C(O)—C₁₋₆haloalkyl, S(O)₂—C₁₋₆alkyl, S(O)₂—C₁₋₆haloalkyl; or R^{a} and R^{b} together with the nitrogen to which they are attached form a 5- to 12-membered monocyclic or bicyclic heterocycle optionally further comprising 1-3 additional ring heteroatoms selected from O, N, and S. In some cases, R^{a} and R^{b} are each independently hydrogen, C₁₋₆alkyl, C₁₋₆alkylene-OH, C₁₋₆alkoxy-C₁₋₆alkyl, C(O)—C₁₋₆alkyl, C(O)—C₁₋₆haloalkyl, S(O)₂—C₁₋₆alkyl, S(O)₂—C₁₋₆haloalkyl. In some cases, R^{a} and R^{b} together with the nitrogen to which they are attached form a 5- to 12-membered monocyclic or bicyclic heterocycle optionally further comprising 1-3 additional ring heteroatoms selected from O, N, and S. In some cases, R^{a} and R^{b} are each independently C₁₋₆ haloalkyl.

In some cases, at least one R^{d} is H. In some cases, each R^{d} is H. In some cases, at least one R^{d} is D. In some cases, each R^{d} is D. In some cases, at least one R^{e} is H. In some cases, each R^{e} is H. In some cases, at least one R^{e} is D. In some cases, each R^{e} is D. In some cases, at least one R^{e} is OH. In some cases, each R^{e} is OH. In some cases, at least one R^{e} is halo. In some cases, at least one R^{e} is F. In some cases, each R^{e} is halo. In some cases, each R^{e} is F. In some cases, two geminal R^{e} together with the atom to which they are attached form an oxo group.

In some cases, R⁴ is C₂₋₆alkenyl, C₂₋₆alkynyl, C₀₋₆alkylene-C₃₋₆cycloalkyl, or C₀₋₆alkylene-C₆₋₁₀aryl. In some cases, R⁴ is C₂₋₆alkynyl, C₀₋₆alkylene-C₃₋₆cycloalkyl, or C₀₋₆alkylene-C₆₋₁₀aryl. In some cases, R⁴ is C₂₋₆alkenyl. In some cases, R⁴ is C₂₋₆alkynyl. In some cases, R⁴ is propargyl. In some cases, R⁴ is C₀₋₆alkylene-C₃₋₆cycloalkyl. In some cases, R⁴ is C₀₋₆alkylene-cyclopropyl. In some cases, R⁴ is C₀₋₆alkylene-cyclobutyl. In some cases, R⁴ is C₀₋₆alkylene-C₆₋₁₀aryl. In some cases, R⁴ is C₀₋₆alkylene-C₆aryl. In some cases, R⁴ is phenyl.

In some cases, R⁴ is benzyl.

In some cases, n is 1 or 2. In some cases, n is 0. In some cases, n is 1. In some cases, n is 2.

In some cases, R² is F or Cl. In some cases, R² is F.

In some cases, R¹ is H. In some cases, R¹ is halo. In some cases, R¹ is F.

Specific compounds contemplated include those listed in Table A, or a pharmaceutically acceptable salt thereof:

TABLE A

| Structure | Cpd No. |
|---|---|
| 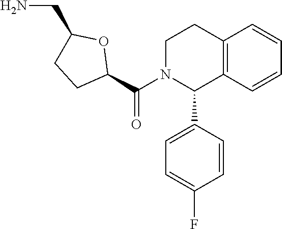 [SMILES: NC[C@@H]1CC[C@H](C(=O)N2CCc3ccccc3[C@@H]2c2ccc(F)cc2)O1] | 5000 |
| 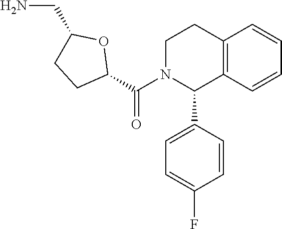 [SMILES: NC[C@H]1CC[C@@H](C(=O)N2CCc3ccccc3[C@@H]2c2ccc(F)cc2)O1] | 5001 |
| 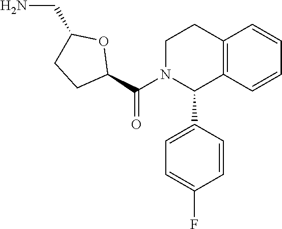 [SMILES: NC[C@H]1CC[C@H](C(=O)N2CCc3ccccc3[C@@H]2c2ccc(F)cc2)O1] | 5002 |
| 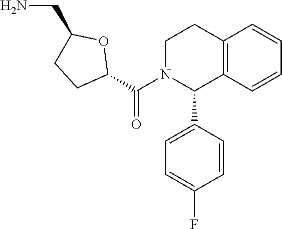 [SMILES: NC[C@@H]1CC[C@@H](C(=O)N2CCc3ccccc3[C@@H]2c2ccc(F)cc2)O1] | 5003 |
| 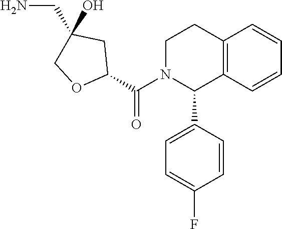 [SMILES: NC[C@@]1(O)CO[C@@H](C(=O)N2CCc3ccccc3[C@@H]2c2ccc(F)cc2)C1] | 5004 |
| 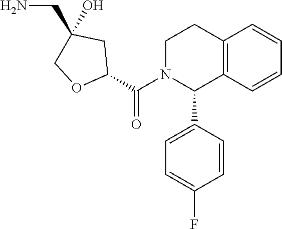 [SMILES: NC[C@]1(O)CO[C@@H](C(=O)N2CCc3ccccc3[C@@H]2c2ccc(F)cc2)C1] | 5005 |
| 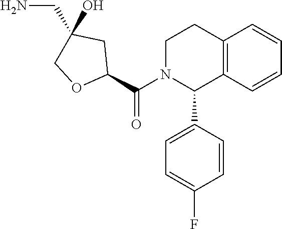 [SMILES: NC[C@@]1(O)CO[C@H](C(=O)N2CCc3ccccc3[C@@H]2c2ccc(F)cc2)C1] | 5006 |
| 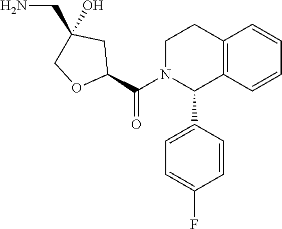 [SMILES: NC[C@]1(O)CO[C@H](C(=O)N2CCc3ccccc3[C@@H]2c2ccc(F)cc2)C1] | 5007 |
| 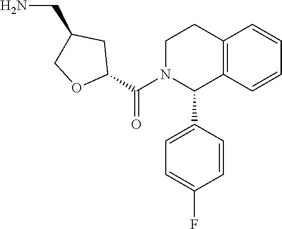 [SMILES: NC[C@@H]1CO[C@@H](C(=O)N2CCc3ccccc3[C@@H]2c2ccc(F)cc2)C1] | 5008 |
| 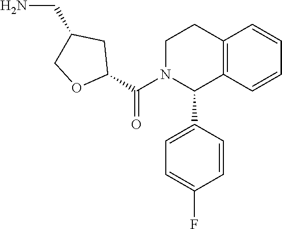 [SMILES: NC[C@H]1CO[C@@H](C(=O)N2CCc3ccccc3[C@@H]2c2ccc(F)cc2)C1] | 5009 |
| 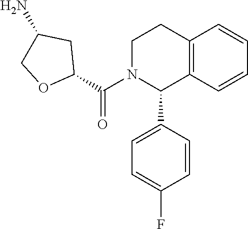 [SMILES: N[C@H]1CO[C@@H](C(=O)N2CCc3ccccc3[C@@H]2c2ccc(F)cc2)C1] | 5010 |
| 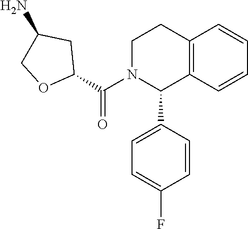 [SMILES: N[C@@H]1CO[C@@H](C(=O)N2CCc3ccccc3[C@@H]2c2ccc(F)cc2)C1] | 5011 |
| 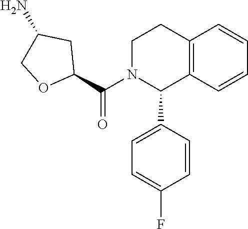 [SMILES: N[C@H]1CO[C@H](C(=O)N2CCc3ccccc3[C@@H]2c2ccc(F)cc2)C1] | 5012 |
| 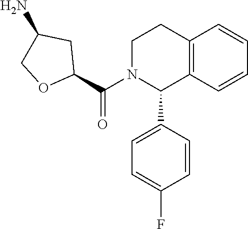 [SMILES: N[C@@H]1CO[C@H](C(=O)N2CCc3ccccc3[C@@H]2c2ccc(F)cc2)C1] | 5013 |
| 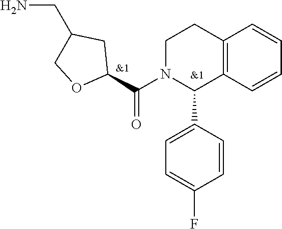 [SMILES: NCC1CO[C@H](C(=O)N2CCc3ccccc3[C@@H]2c2ccc(F)cc2)C1] | 5014 |
| 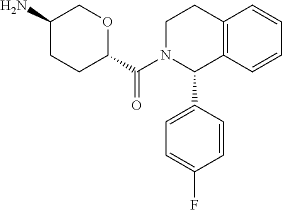 [SMILES: N[C@@H]1CC[C@@H](C(=O)N2CCc3ccccc3[C@@H]2c2ccc(F)cc2)OC1] | 5015 |
| 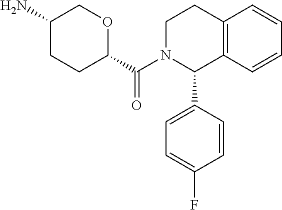 [SMILES: N[C@H]1CC[C@@H](C(=O)N2CCc3ccccc3[C@@H]2c2ccc(F)cc2)OC1] | 5016 |
| 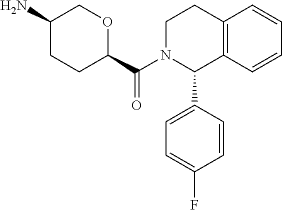 [SMILES: N[C@@H]1CC[C@H](C(=O)N2CCc3ccccc3[C@@H]2c2ccc(F)cc2)OC1] | 5017 |
| 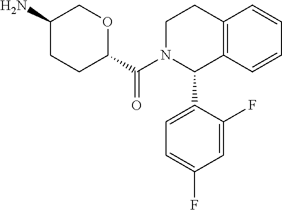 [SMILES: N[C@@H]1CC[C@@H](C(=O)N2CCc3ccccc3[C@@H]2c2ccc(F)cc2F)OC1] | 5018 |
| 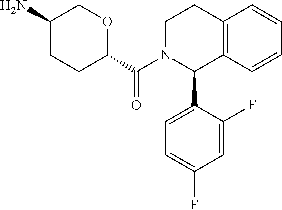 [SMILES: N[C@@H]1CC[C@@H](C(=O)N2CCc3ccccc3[C@H]2c2ccc(F)cc2F)OC1] | 5019 |
| 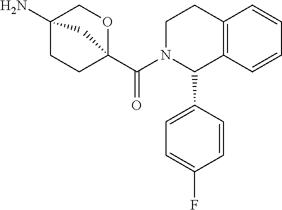 | 5020 |
| 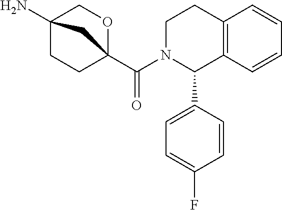 [SMILES: N[C@]12CC[C@](C(=O)N3CCc4ccccc4[C@@H]3c3ccc(F)cc3)(C1)OC2] | 5021 |
| 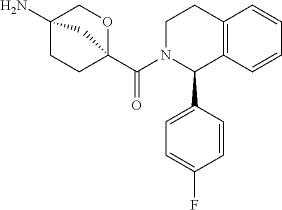 [SMILES: N[C@@]12CC[C@@](C(=O)N3CCc4ccccc4[C@H]3c3ccc(F)cc3)(C1)OC2] | 5022 |
| 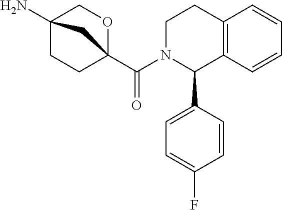 [SMILES: N[C@]12CC[C@](C(=O)N3CCc4ccccc4[C@H]3c3ccc(F)cc3)(C1)OC2] | 5023 |
| 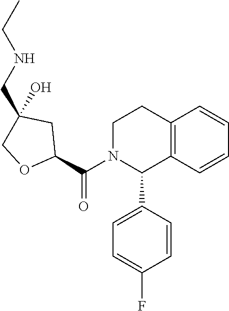 [SMILES: CCNC[C@]1(O)CO[C@H](C(=O)N2CCc3ccccc3[C@@H]2c2ccc(F)cc2)C1] | 5024 |
| 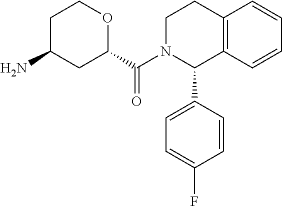 [SMILES: N[C@H]1CCO[C@H](C(=O)N2CCc3ccccc3[C@@H]2c2ccc(F)cc2)C1] | 5027 |
| 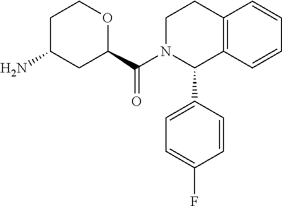 [SMILES: N[C@@H]1CCO[C@@H](C(=O)N2CCc3ccccc3[C@@H]2c2ccc(F)cc2)C1] | 5028 |
| 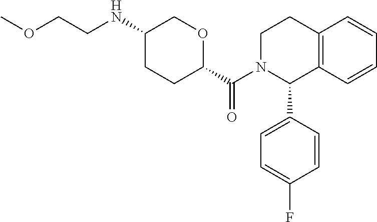 [SMILES: COCCN[C@H]1CC[C@@H](C(=O)N2CCc3ccccc3[C@@H]2c2ccc(F)cc2)OC1] | 5029 |
| 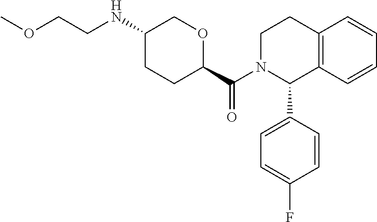 [SMILES: COCCN[C@H]1CC[C@H](C(=O)N2CCc3ccccc3[C@@H]2c2ccc(F)cc2)OC1] | 5030 |
| 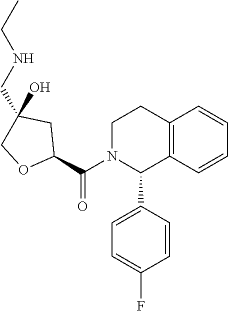 [SMILES: CCNC[C@@]1(O)CO[C@H](C(=O)N2CCc3ccccc3[C@@H]2c2ccc(F)cc2)C1] | 5031 |
| 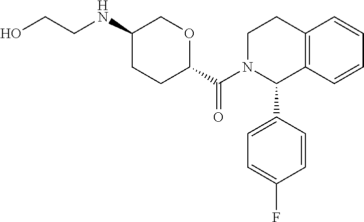 [SMILES: O=C([C@@H]1CC[C@@H](NCCO)CO1)N1CCc2ccccc2[C@@H]1c1ccc(F)cc1] | 5032 |
| 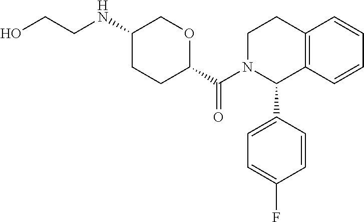 [SMILES: O=C([C@@H]1CC[C@H](NCCO)CO1)N1CCc2ccccc2[C@@H]1c1ccc(F)cc1] | 5033 |
| 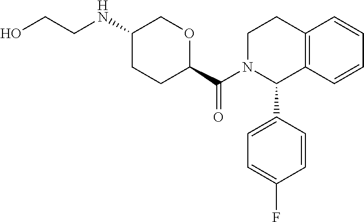 [SMILES: O=C([C@H]1CC[C@H](NCCO)CO1)N1CCc2ccccc2[C@@H]1c1ccc(F)cc1] | 5034 |
| 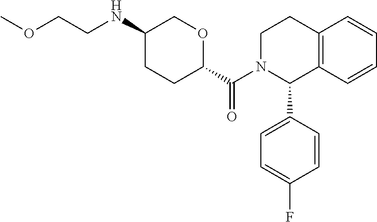 [SMILES: COCCN[C@@H]1CC[C@@H](C(=O)N2CCc3ccccc3[C@@H]2c2ccc(F)cc2)OC1] | 5035 |
| 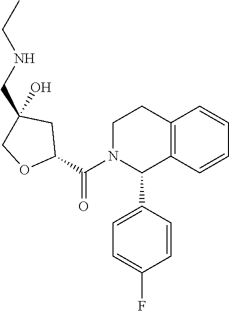 [SMILES: CCNC[C@]1(O)CO[C@@H](C(=O)N2CCc3ccccc3[C@@H]2c2ccc(F)cc2)C1] | 5036 |
| 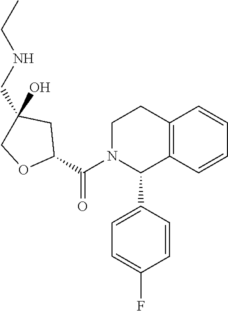 [SMILES: CCNC[C@@]1(O)CO[C@@H](C(=O)N2CCc3ccccc3[C@@H]2c2ccc(F)cc2)C1] | 5037 |
| 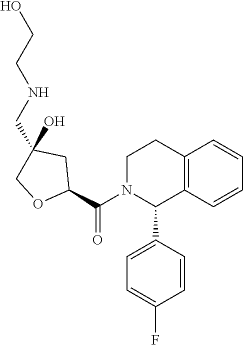 [SMILES: O=C([C@@H]1C[C@@](O)(CNCCO)CO1)N1CCc2ccccc2[C@@H]1c1ccc(F)cc1] | 5038 |
| 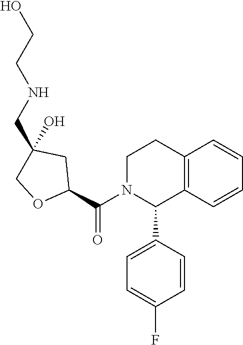 [SMILES: O=C([C@@H]1C[C@](O)(CNCCO)CO1)N1CCc2ccccc2[C@@H]1c1ccc(F)cc1] | 5039 |
| 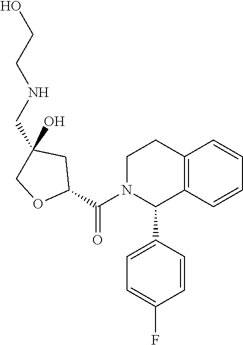 [SMILES: O=C([C@H]1C[C@@](O)(CNCCO)CO1)N1CCc2ccccc2[C@@H]1c1ccc(F)cc1] | 5040 |
| 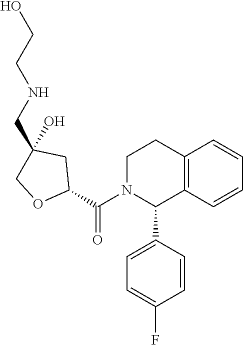 [SMILES: O=C([C@H]1C[C@](O)(CNCCO)CO1)N1CCc2ccccc2[C@@H]1c1ccc(F)cc1] | 5041 |
| 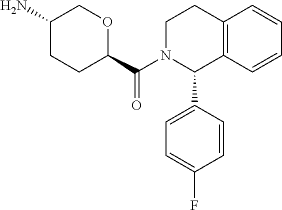 [SMILES: N[C@H]1CC[C@H](C(=O)N2CCc3ccccc3[C@@H]2c2ccc(F)cc2)OC1] | 5042 |
| 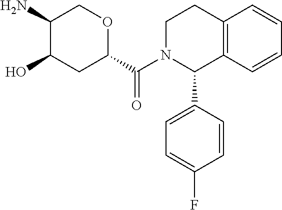 [SMILES: N[C@H]1CO[C@H](C(=O)N2CCc3ccccc3[C@@H]2c2ccc(F)cc2)C[C@H]1O] | 5043 |
| 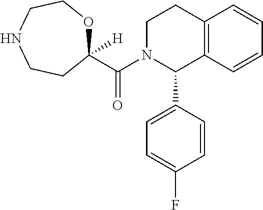 [SMILES: O=C([C@H]1CCNCCO1)N1CCc2ccccc2[C@@H]1c1ccc(F)cc1] | 5044 |
| 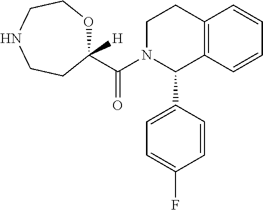 [SMILES: O=C([C@@H]1CCNCCO1)N1CCc2ccccc2[C@@H]1c1ccc(F)cc1] | 5045 |
| 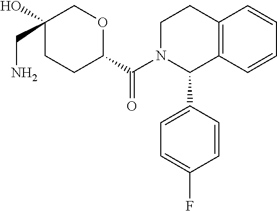 [SMILES: NC[C@]1(O)CC[C@@H](C(=O)N2CCc3ccccc3[C@@H]2c2ccc(F)cc2)OC1] | 5046 |
| 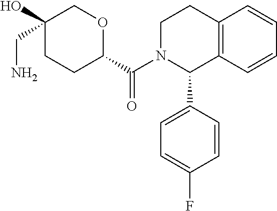 [SMILES: NC[C@@]1(O)CC[C@@H](C(=O)N2CCc3ccccc3[C@@H]2c2ccc(F)cc2)OC1] | 5047 |
| 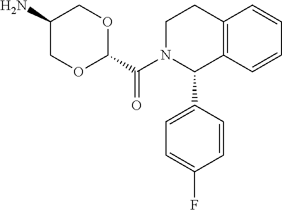 [SMILES: N[C@H]1CO[C@H](C(=O)N2CCc3ccccc3[C@@H]2c2ccc(F)cc2)OC1] | 5048 |
| 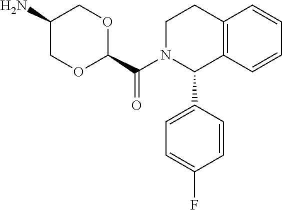 [SMILES: N[C@H]1CO[C@@H](C(=O)N2CCc3ccccc3[C@@H]2c2ccc(F)cc2)OC1] | 5049 |
| 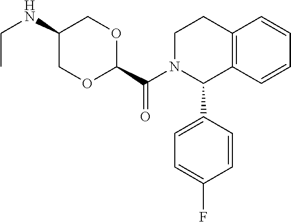 [SMILES: CCN[C@H]1CO[C@@H](C(=O)N2CCc3ccccc3[C@@H]2c2ccc(F)cc2)OC1] | 5050 |
| 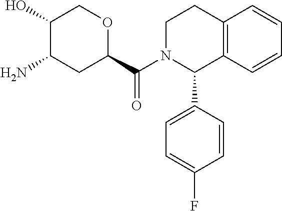 [SMILES: N[C@H]1C[C@H](C(=O)N2CCc3ccccc3[C@@H]2c2ccc(F)cc2)OC[C@H]1O] | 5051 |
| 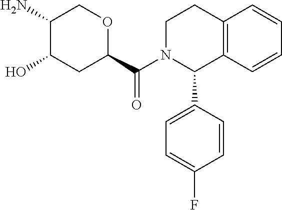 [SMILES: N[C@@H]1CO[C@@H](C(=O)N2CCc3ccccc3[C@@H]2c2ccc(F)cc2)C[C@@H]1O] | 5052 |
| 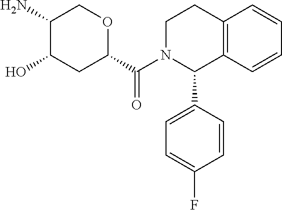 [SMILES: N[C@@H]1CO[C@H](C(=O)N2CCc3ccccc3[C@@H]2c2ccc(F)cc2)C[C@@H]1O] | 5053 |
| 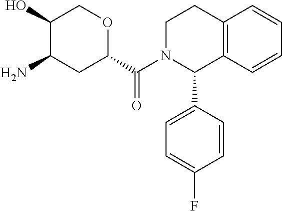 [SMILES: N[C@@H]1C[C@@H](C(=O)N2CCc3ccccc3[C@@H]2c2ccc(F)cc2)OC[C@@H]1O] | 5054 |
| 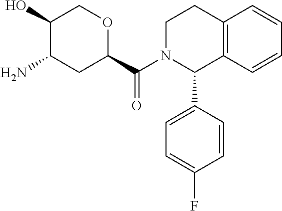 [SMILES: N[C@H]1C[C@H](C(=O)N2CCc3ccccc3[C@@H]2c2ccc(F)cc2)OC[C@@H]1O] | 5055 |
| 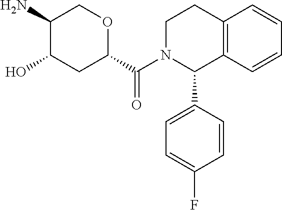 [SMILES: N[C@H]1CO[C@H](C(=O)N2CCc3ccccc3[C@@H]2c2ccc(F)cc2)C[C@@H]1O] | 5056 |
| 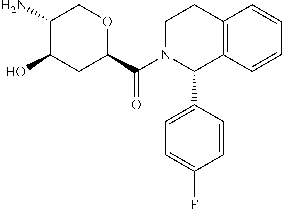 [SMILES: N[C@@H]1CO[C@@H](C(=O)N2CCc3ccccc3[C@@H]2c2ccc(F)cc2)C[C@H]1O] | 5057 |
| 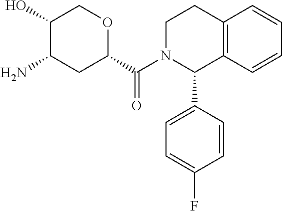 [SMILES: N[C@H]1C[C@@H](C(=O)N2CCc3ccccc3[C@@H]2c2ccc(F)cc2)OC[C@H]1O] | 5058 |
| 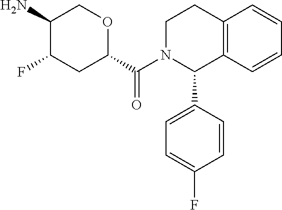 [SMILES: N[C@H]1CO[C@H](C(=O)N2CCc3ccccc3[C@@H]2c2ccc(F)cc2)C[C@@H]1F] | 5059 |
| 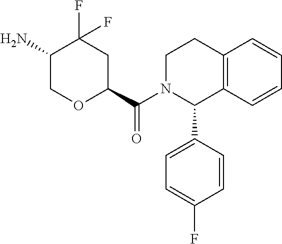 [SMILES: N[C@H]1CO[C@H](C(=O)N2CCc3ccccc3[C@@H]2c2ccc(F)cc2)CC1(F)F] | 5060 |
| 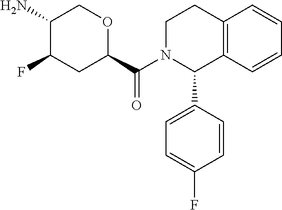 [SMILES: N[C@@H]1CO[C@@H](C(=O)N2CCc3ccccc3[C@@H]2c2ccc(F)cc2)C[C@H]1F] | 5061 |
| 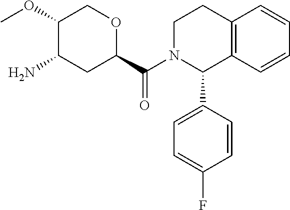 [SMILES: CO[C@@H]1CO[C@@H](C(=O)N2CCc3ccccc3[C@@H]2c2ccc(F)cc2)C[C@@H]1N] | 5062 |
| 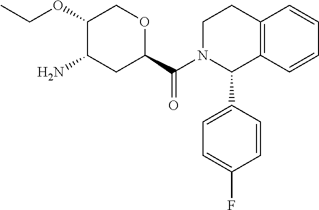 [SMILES: CCO[C@@H]1CO[C@@H](C(=O)N2CCc3ccccc3[C@@H]2c2ccc(F)cc2)C[C@@H]1N] | 5063 |
| 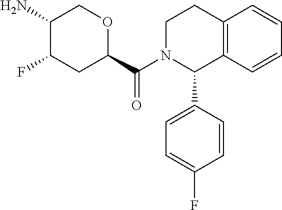 [SMILES: N[C@@H]1CO[C@@H](C(=O)N2CCc3ccccc3[C@@H]2c2ccc(F)cc2)C[C@@H]1F] | 5064 |
| 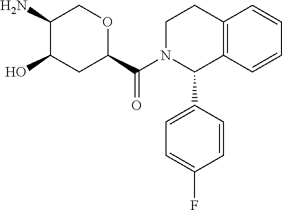 [SMILES: N[C@H]1CO[C@@H](C(=O)N2CCc3ccccc3[C@@H]2c2ccc(F)cc2)C[C@H]1O] | 5065 |
| 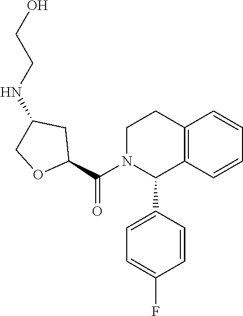 [SMILES: O=C([C@@H]1C[C@@H](NCCO)CO1)N1CCc2ccccc2[C@@H]1c1ccc(F)cc1] | 5066 |
| 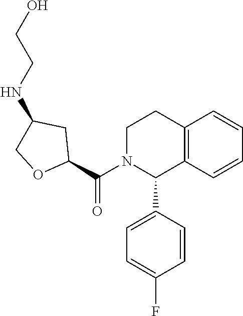 [SMILES: O=C([C@@H]1C[C@H](NCCO)CO1)N1CCc2ccccc2[C@@H]1c1ccc(F)cc1] | 5067 |
| 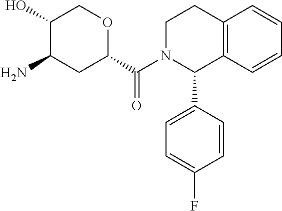 [SMILES: N[C@@H]1C[C@@H](C(=O)N2CCc3ccccc3[C@@H]2c2ccc(F)cc2)OC[C@H]1O] | 5068 |
| 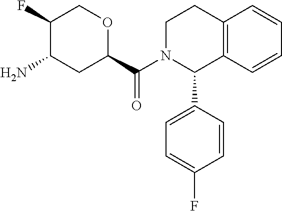 [SMILES: N[C@H]1C[C@H](C(=O)N2CCc3ccccc3[C@@H]2c2ccc(F)cc2)OC[C@@H]1F] | 5069 |
| 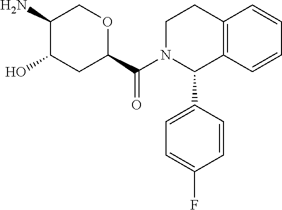 [SMILES: N[C@H]1CO[C@@H](C(=O)N2CCc3ccccc3[C@@H]2c2ccc(F)cc2)C[C@@H]1O] | 5070 |
| 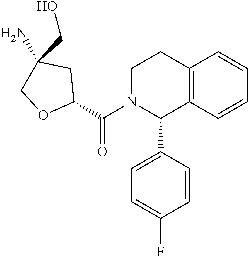 [SMILES: N[C@@]1(CO)CO[C@@H](C(=O)N2CCc3ccccc3[C@@H]2c2ccc(F)cc2)C1] | 5071 |
| 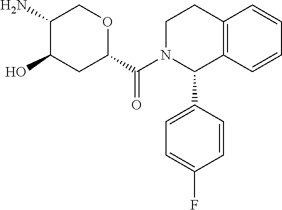 [SMILES: N[C@@H]1CO[C@H](C(=O)N2CCc3ccccc3[C@@H]2c2ccc(F)cc2)C[C@H]1O] | 5072 |
| 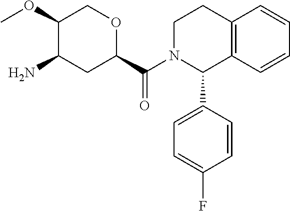 [SMILES: CO[C@H]1CO[C@@H](C(=O)N2CCc3ccccc3[C@@H]2c2ccc(F)cc2)C[C@H]1N] | 5073 |
| 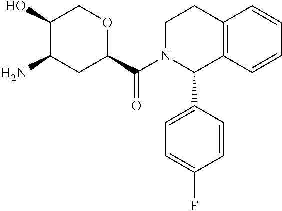 [SMILES: N[C@@H]1C[C@H](C(=O)N2CCc3ccccc3[C@@H]2c2ccc(F)cc2)OC[C@@H]1O] | 5074 |
| 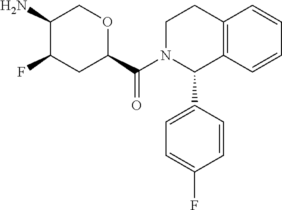 [SMILES: N[C@H]1CO[C@@H](C(=O)N2CCc3ccccc3[C@@H]2c2ccc(F)cc2)C[C@H]1F] | 5075 |
| 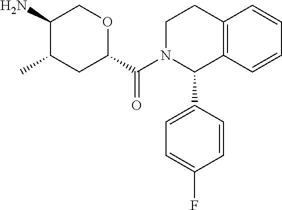 [SMILES: C[C@H]1C[C@@H](C(=O)N2CCc3ccccc3[C@@H]2c2ccc(F)cc2)OC[C@@H]1N] | 5076 |
| 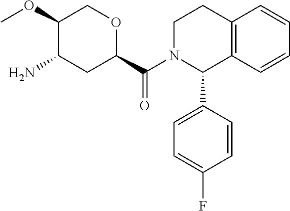 [SMILES: CO[C@H]1CO[C@@H](C(=O)N2CCc3ccccc3[C@@H]2c2ccc(F)cc2)C[C@@H]1N] | 5077 |
| 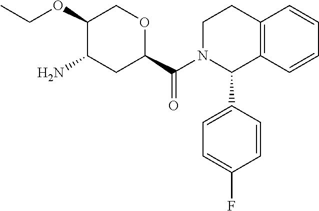 [SMILES: CCO[C@H]1CO[C@@H](C(=O)N2CCc3ccccc3[C@@H]2c2ccc(F)cc2)C[C@@H]1N] | 5078 |
| 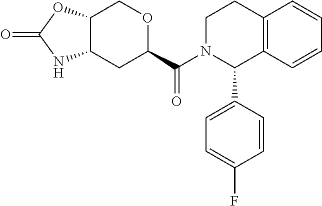 [SMILES: O=C1N[C@H]2C[C@H](C(=O)N3CCc4ccccc4[C@@H]3c3ccc(F)cc3)OC[C@H]2O1] | 5079 |
| 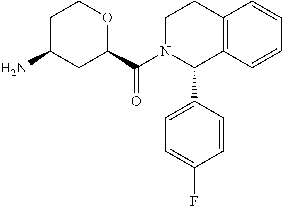 [SMILES: N[C@H]1CCO[C@@H](C(=O)N2CCc3ccccc3[C@@H]2c2ccc(F)cc2)C1] | 5080 |
| 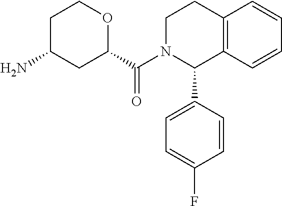 [SMILES: N[C@@H]1CCO[C@H](C(=O)N2CCc3ccccc3[C@@H]2c2ccc(F)cc2)C1] | 5081 |
| 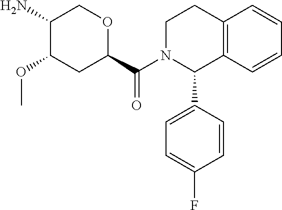 [SMILES: CO[C@H]1C[C@H](C(=O)N2CCc3ccccc3[C@@H]2c2ccc(F)cc2)OC[C@H]1N] | 5082 |
| 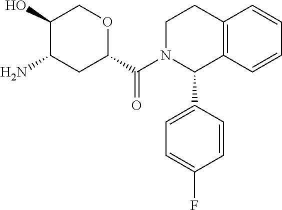 [SMILES: N[C@H]1C[C@@H](C(=O)N2CCc3ccccc3[C@@H]2c2ccc(F)cc2)OC[C@@H]1O] | 5083 |
| 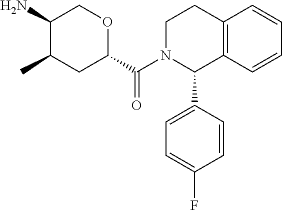 [SMILES: C[C@@H]1C[C@@H](C(=O)N2CCc3ccccc3[C@@H]2c2ccc(F)cc2)OC[C@@H]1N] | 5084 |
| 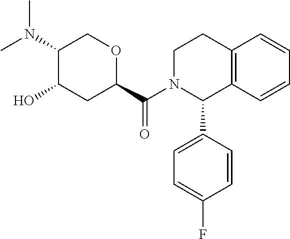 [SMILES: CN(C)[C@@H]1CO[C@@H](C(=O)N2CCc3ccccc3[C@@H]2c2ccc(F)cc2)C[C@@H]1O] | 5085 |
| 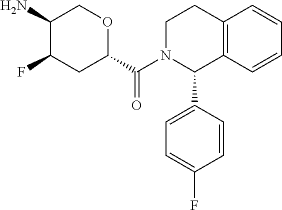 [SMILES: N[C@H]1CO[C@H](C(=O)N2CCc3ccccc3[C@@H]2c2ccc(F)cc2)C[C@H]1F] | 5086 |
| 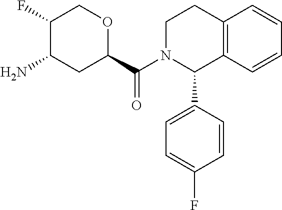 [SMILES: N[C@H]1C[C@H](C(=O)N2CCc3ccccc3[C@@H]2c2ccc(F)cc2)OC[C@H]1F] | 5087 |
| 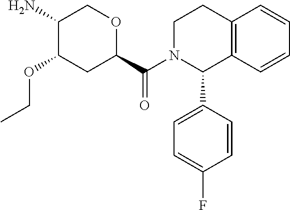 [SMILES: CCO[C@H]1C[C@H](C(=O)N2CCc3ccccc3[C@@H]2c2ccc(F)cc2)OC[C@H]1N] | 5088 |
| 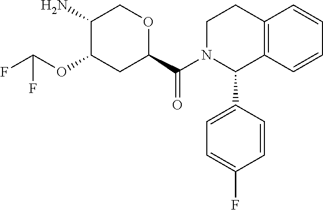 [SMILES: N[C@@H]1CO[C@@H](C(=O)N2CCc3ccccc3[C@@H]2c2ccc(F)cc2)C[C@@H]1OC(F)F] | 5089 |
| 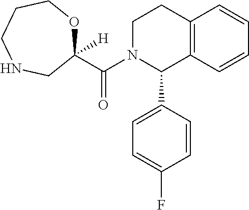 [SMILES: O=C([C@H]1CNCCCO1)N1CCc2ccccc2[C@@H]1c1ccc(F)cc1] | 5090 |
| 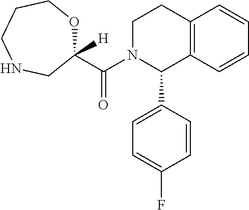 [SMILES: O=C([C@@H]1CNCCCO1)N1CCc2ccccc2[C@@H]1c1ccc(F)cc1] | 5091 |
| 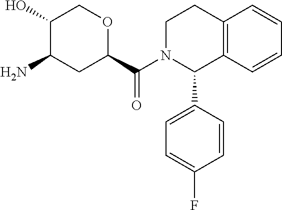 [SMILES: N[C@@H]1C[C@H](C(=O)N2CCc3ccccc3[C@@H]2c2ccc(F)cc2)OC[C@H]1O] | 5092 |
| 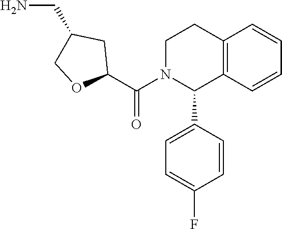 [SMILES: NC[C@H]1CO[C@H](C(=O)N2CCc3ccccc3[C@@H]2c2ccc(F)cc2)C1] | 5200 |
| 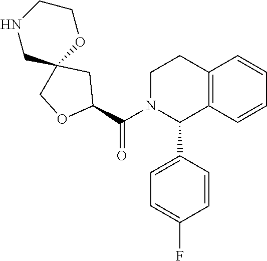 [SMILES: O=C([C@@H]1C[C@]2(CNCCO2)CO1)N1CCc2ccccc2[C@@H]1c1ccc(F)cc1] | 5201 |
| 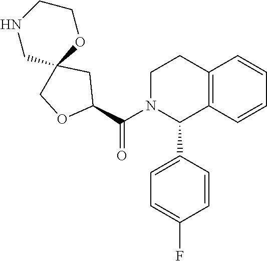 [SMILES: O=C([C@@H]1C[C@@]2(CNCCO2)CO1)N1CCc2ccccc2[C@@H]1c1ccc(F)cc1] | 5202 |
| 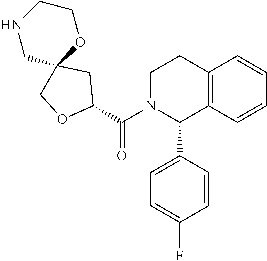 [SMILES: O=C([C@H]1C[C@@]2(CNCCO2)CO1)N1CCc2ccccc2[C@@H]1c1ccc(F)cc1] | 5203 |
| 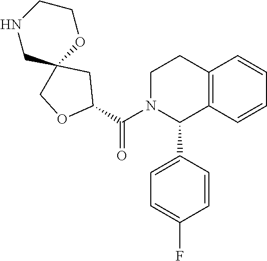 [SMILES: O=C([C@H]1C[C@]2(CNCCO2)CO1)N1CCc2ccccc2[C@@H]1c1ccc(F)cc1] | 5204 |
| 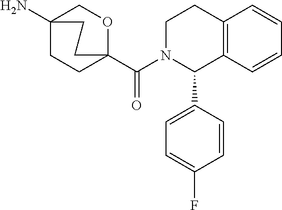 [SMILES: NC12CCC(C(=O)N3CCc4ccccc4[C@@H]3c3ccc(F)cc3)(CC1)OC2] | 5205 |
| 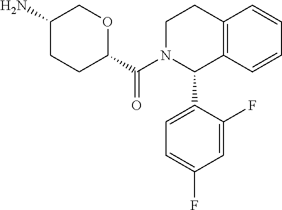 [SMILES: N[C@H]1CC[C@@H](C(=O)N2CCc3ccccc3[C@@H]2c2ccc(F)cc2F)OC1] | 5206 |
| 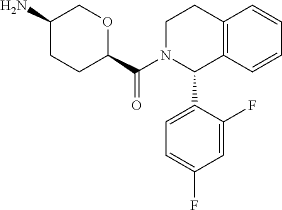 [SMILES: N[C@@H]1CC[C@H](C(=O)N2CCc3ccccc3[C@@H]2c2ccc(F)cc2F)OC1] | 5207 |
| 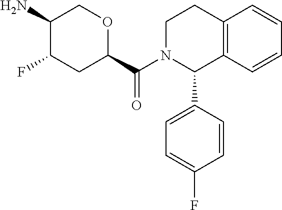 [SMILES: N[C@H]1CO[C@@H](C(=O)N2CCc3ccccc3[C@@H]2c2ccc(F)cc2)C[C@@H]1F] | 5210 |
| 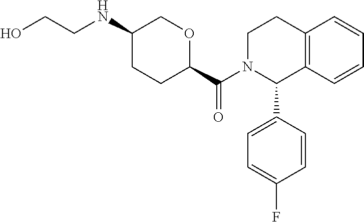 [SMILES: O=C([C@H]1CC[C@@H](NCCO)CO1)N1CCc2ccccc2[C@@H]1c1ccc(F)cc1] | 5211 |
| 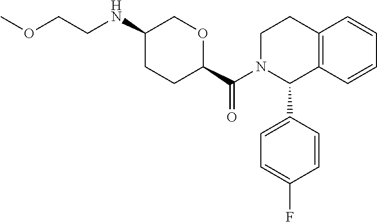 [SMILES: COCCN[C@@H]1CC[C@H](C(=O)N2CCc3ccccc3[C@@H]2c2ccc(F)cc2)OC1] | 5212 |
| 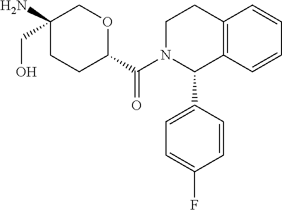 [SMILES: N[C@]1(CO)CC[C@@H](C(=O)N2CCc3ccccc3[C@@H]2c2ccc(F)cc2)OC1] | 5213 |
| 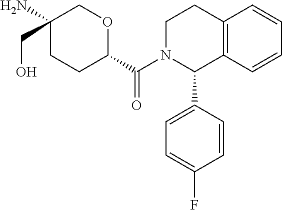 [SMILES: N[C@@]1(CO)CC[C@@H](C(=O)N2CCc3ccccc3[C@@H]2c2ccc(F)cc2)OC1] | 5214 |
| 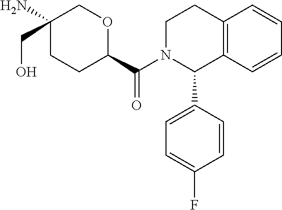 [SMILES: N[C@@]1(CO)CC[C@H](C(=O)N2CCc3ccccc3[C@@H]2c2ccc(F)cc2)OC1] | 5215 |
| 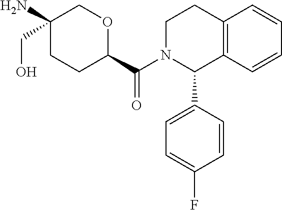 [SMILES: N[C@]1(CO)CC[C@H](C(=O)N2CCc3ccccc3[C@@H]2c2ccc(F)cc2)OC1] | 5216 |
| 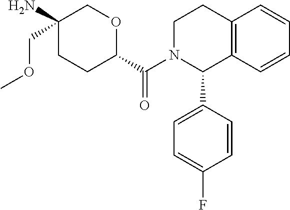 [SMILES: COC[C@@]1(N)CC[C@@H](C(=O)N2CCc3ccccc3[C@@H]2c2ccc(F)cc2)OC1] | 5217 |
| 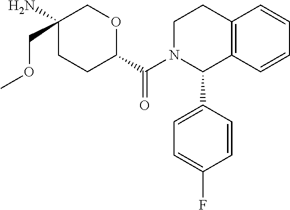 [SMILES: COC[C@]1(N)CC[C@@H](C(=O)N2CCc3ccccc3[C@@H]2c2ccc(F)cc2)OC1] | 5218 |
| 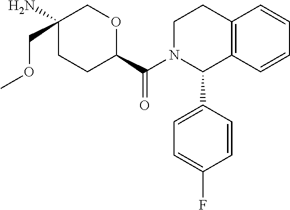 [SMILES: COC[C@]1(N)CC[C@H](C(=O)N2CCc3ccccc3[C@@H]2c2ccc(F)cc2)OC1] | 5219 |
| 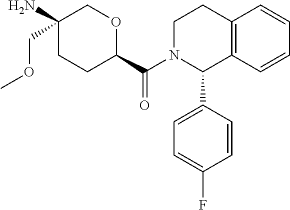 [SMILES: COC[C@@]1(N)CC[C@H](C(=O)N2CCc3ccccc3[C@@H]2c2ccc(F)cc2)OC1] | 5220 |
| 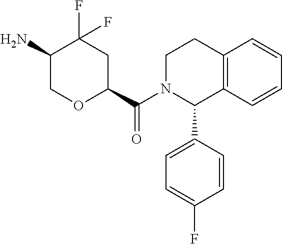 [SMILES: N[C@@H]1CO[C@H](C(=O)N2CCc3ccccc3[C@@H]2c2ccc(F)cc2)CC1(F)F] | 5221 |
| 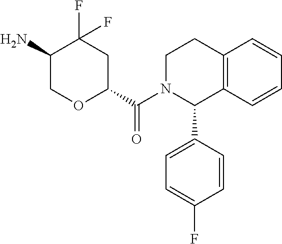 [SMILES: N[C@@H]1CO[C@@H](C(=O)N2CCc3ccccc3[C@@H]2c2ccc(F)cc2)CC1(F)F] | 5222 |
| 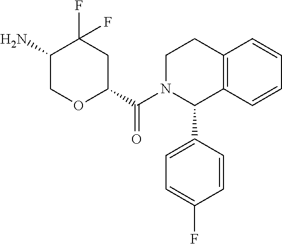 [SMILES: N[C@H]1CO[C@@H](C(=O)N2CCc3ccccc3[C@@H]2c2ccc(F)cc2)CC1(F)F] | 5223 |
| 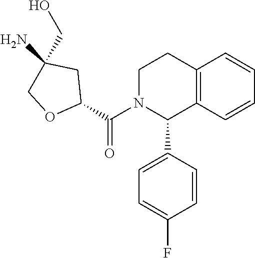 [SMILES: N[C@]1(CO)CO[C@@H](C(=O)N2CCc3ccccc3[C@@H]2c2ccc(F)cc2)C1] | 5224 |
| 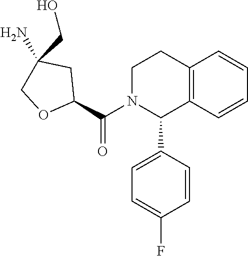 [SMILES: N[C@@]1(CO)CO[C@H](C(=O)N2CCc3ccccc3[C@@H]2c2ccc(F)cc2)C1] | 5225 |
| 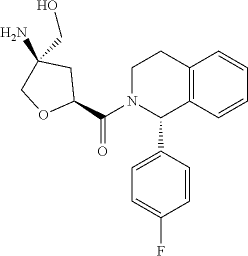 [SMILES: N[C@]1(CO)CO[C@H](C(=O)N2CCc3ccccc3[C@@H]2c2ccc(F)cc2)C1] | 5226 |
| 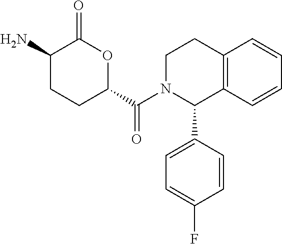 [SMILES: N[C@@H]1CC[C@@H](C(=O)N2CCc3ccccc3[C@@H]2c2ccc(F)cc2)OC1=O] | 5227 |
| 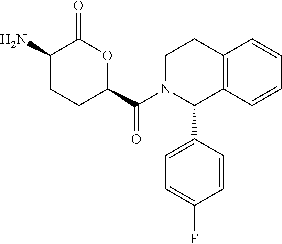 [SMILES: N[C@@H]1CC[C@H](C(=O)N2CCc3ccccc3[C@@H]2c2ccc(F)cc2)OC1=O] | 5228 |
| 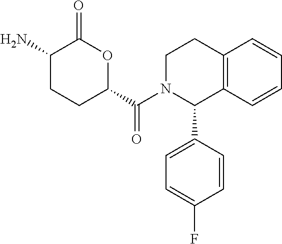 [SMILES: N[C@H]1CC[C@@H](C(=O)N2CCc3ccccc3[C@@H]2c2ccc(F)cc2)OC1=O] | 5229 |
| 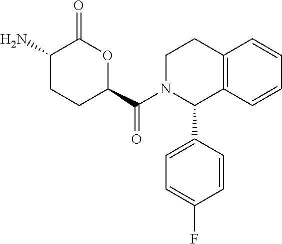 [SMILES: N[C@H]1CC[C@H](C(=O)N2CCc3ccccc3[C@@H]2c2ccc(F)cc2)OC1=O] | 5230 |
| 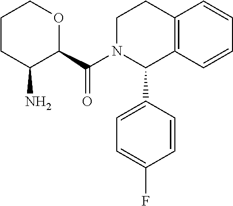 [SMILES: N[C@H]1CCCO[C@H]1C(=O)N1CCc2ccccc2[C@@H]1c1ccc(F)cc1] | 5231 |
| 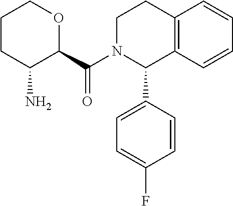 [SMILES: N[C@@H]1CCCO[C@H]1C(=O)N1CCc2ccccc2[C@@H]1c1ccc(F)cc1] | 5232 |
| 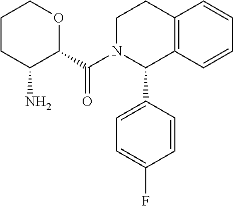 [SMILES: N[C@@H]1CCCO[C@@H]1C(=O)N1CCc2ccccc2[C@@H]1c1ccc(F)cc1] | 5233 |
| 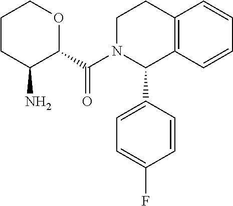 [SMILES: N[C@H]1CCCO[C@@H]1C(=O)N1CCc2ccccc2[C@@H]1c1ccc(F)cc1] | 5234 |
| 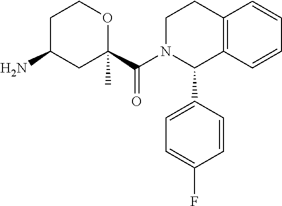 [SMILES: C[C@]1(C(=O)N2CCc3ccccc3[C@@H]2c2ccc(F)cc2)C[C@@H](N)CCO1] | 5235 |
| 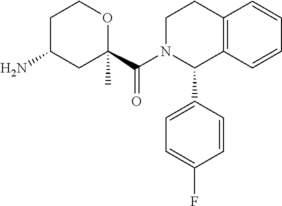 [SMILES: C[C@]1(C(=O)N2CCc3ccccc3[C@@H]2c2ccc(F)cc2)C[C@H](N)CCO1] | 5236 |
| 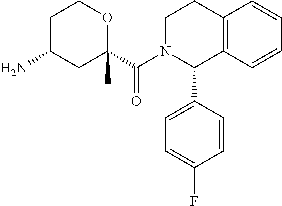 [SMILES: C[C@@]1(C(=O)N2CCc3ccccc3[C@@H]2c2ccc(F)cc2)C[C@H](N)CCO1] | 5237 |
| 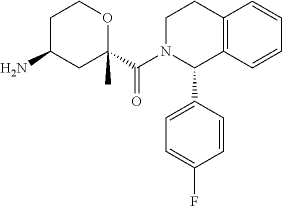 [SMILES: C[C@@]1(C(=O)N2CCc3ccccc3[C@@H]2c2ccc(F)cc2)C[C@@H](N)CCO1] | 5238 |
| 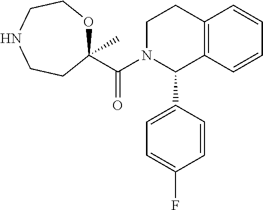 [SMILES: C[C@]1(C(=O)N2CCc3ccccc3[C@@H]2c2ccc(F)cc2)CCNCCO1] | 5239 |
| 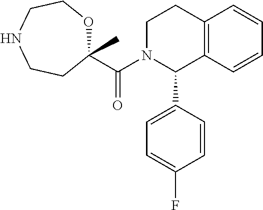 [SMILES: C[C@@]1(C(=O)N2CCc3ccccc3[C@@H]2c2ccc(F)cc2)CCNCCO1] | 5240 |
| 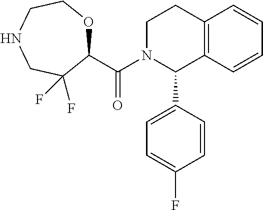 [SMILES: O=C([C@@H]1OCCNCC1(F)F)N1CCc2ccccc2[C@@H]1c1ccc(F)cc1] | 5241 |
| 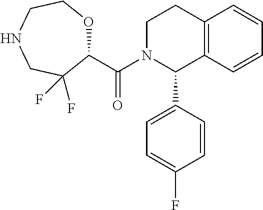 [SMILES: O=C([C@H]1OCCNCC1(F)F)N1CCc2ccccc2[C@@H]1c1ccc(F)cc1] | 5242 |
| 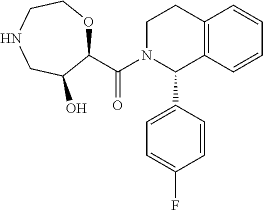 [SMILES: O=C([C@@H]1OCCNC[C@@H]1O)N1CCc2ccccc2[C@@H]1c1ccc(F)cc1] | 5243 |
| 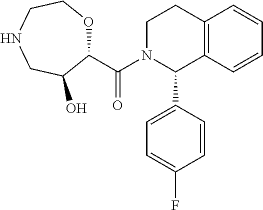 [SMILES: O=C([C@H]1OCCNC[C@@H]1O)N1CCc2ccccc2[C@@H]1c1ccc(F)cc1] | 5244 |
| 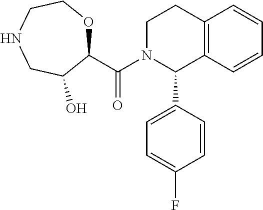 [SMILES: O=C([C@@H]1OCCNC[C@H]1O)N1CCc2ccccc2[C@@H]1c1ccc(F)cc1] | 5245 |
| 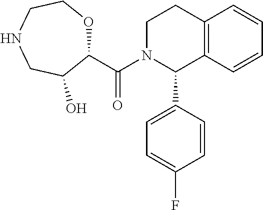 [SMILES: O=C([C@H]1OCCNC[C@H]1O)N1CCc2ccccc2[C@@H]1c1ccc(F)cc1] | 5246 |
| 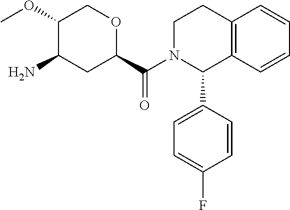 [SMILES: CO[C@@H]1CO[C@@H](C(=O)N2CCc3ccccc3[C@@H]2c2ccc(F)cc2)C[C@H]1N] | 5247 |
| 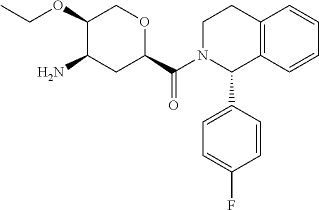 [SMILES: CCO[C@H]1CO[C@@H](C(=O)N2CCc3ccccc3[C@@H]2c2ccc(F)cc2)C[C@H]1N] | 5248 |
| 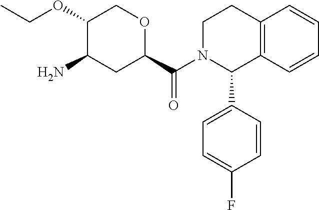 [SMILES: CCO[C@@H]1CO[C@@H](C(=O)N2CCc3ccccc3[C@@H]2c2ccc(F)cc2)C[C@H]1N] | 5249 |
| 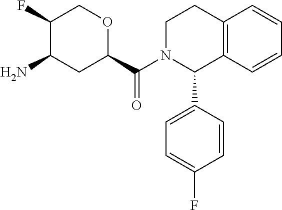 [SMILES: N[C@@H]1C[C@H](C(=O)N2CCc3ccccc3[C@@H]2c2ccc(F)cc2)OC[C@@H]1F] | 5250 |
| 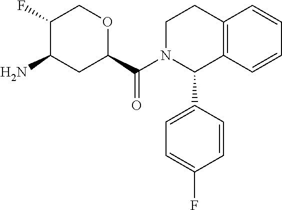 [SMILES: N[C@@H]1C[C@H](C(=O)N2CCc3ccccc3[C@@H]2c2ccc(F)cc2)OC[C@H]1F] | 5251 |
| 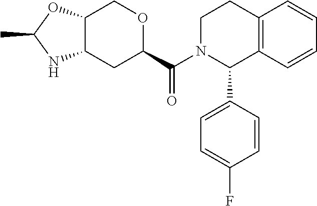 [SMILES: C[C@@H]1N[C@H]2C[C@H](C(=O)N3CCc4ccccc4[C@@H]3c3ccc(F)cc3)OC[C@H]2O1] | 5252 |
| 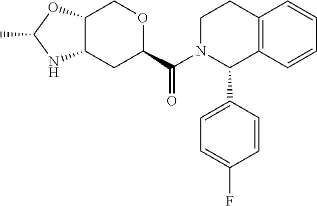 [SMILES: C[C@H]1N[C@H]2C[C@H](C(=O)N3CCc4ccccc4[C@@H]3c3ccc(F)cc3)OC[C@H]2O1] | 5253 |
| 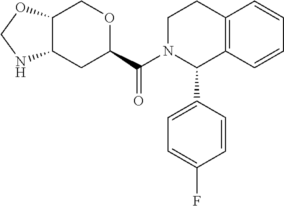 [SMILES: O=C([C@H]1C[C@@H]2NCO[C@@H]2CO1)N1CCc2ccccc2[C@@H]1c1ccc(F)cc1] | 5254 |
| 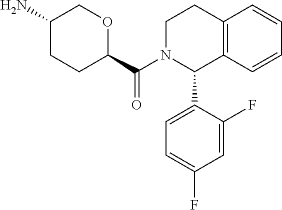 [SMILES: N[C@H]1CC[C@H](C(=O)N2CCc3ccccc3[C@@H]2c2ccc(F)cc2F)OC1] | 5255 |
| 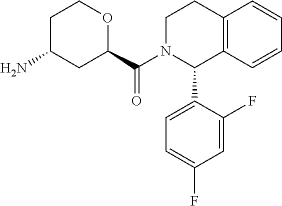 [SMILES: N[C@@H]1CCO[C@@H](C(=O)N2CCc3ccccc3[C@@H]2c2ccc(F)cc2F)C1] | 5256 |
| 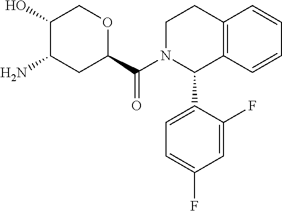 [SMILES: N[C@H]1C[C@H](C(=O)N2CCc3ccccc3[C@@H]2c2ccc(F)cc2F)OC[C@H]1O] | 5257 |
| 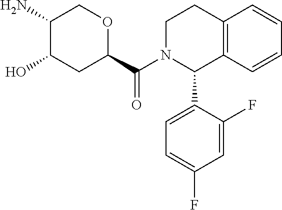 [SMILES: N[C@@H]1CO[C@@H](C(=O)N2CCc3ccccc3[C@@H]2c2ccc(F)cc2F)C[C@@H]1O] | 5258 |
| 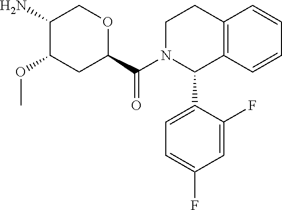 [SMILES: CO[C@H]1C[C@H](C(=O)N2CCc3ccccc3[C@@H]2c2ccc(F)cc2F)OC[C@H]1N] | 5259 |
| 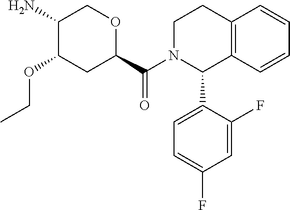 [SMILES: CCO[C@H]1C[C@H](C(=O)N2CCc3ccccc3[C@@H]2c2ccc(F)cc2F)OC[C@H]1N] | 5260 |
| 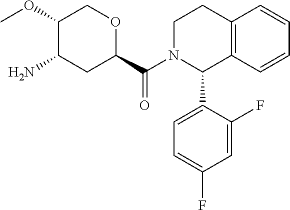 [SMILES: CO[C@@H]1CO[C@@H](C(=O)N2CCc3ccccc3[C@@H]2c2ccc(F)cc2F)C[C@@H]1N] | 5261 |
| 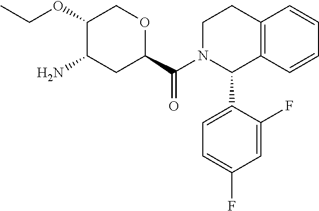 [SMILES: CCO[C@@H]1CO[C@@H](C(=O)N2CCc3ccccc3[C@@H]2c2ccc(F)cc2F)C[C@@H]1N] | 5262 |
| 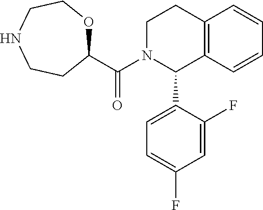 [SMILES: O=C([C@H]1CCNCCO1)N1CCc2ccccc2[C@@H]1c1ccc(F)cc1F] | 5263 |
| 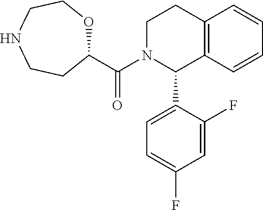 [SMILES: O=C([C@@H]1CCNCCO1)N1CCc2ccccc2[C@@H]1c1ccc(F)cc1F] | 5264 |
| 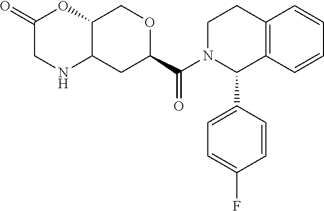 | 5265 |
| 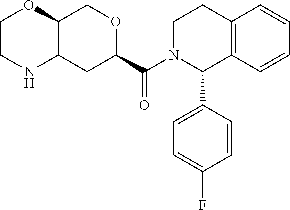 | 5266 |
| 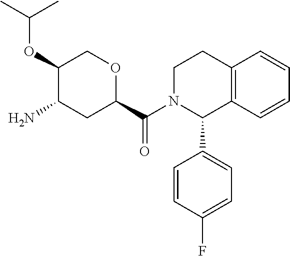 [SMILES: CC(C)O[C@H]1CO[C@@H](C(=O)N2CCc3ccccc3[C@@H]2c2ccc(F)cc2)C[C@@H]1N] | 5267 |
| 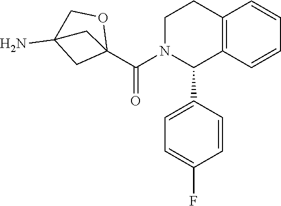 [SMILES: NC12COC(C(=O)N3CCc4ccccc4[C@@H]3c3ccc(F)cc3)(C1)C2] | 5400 |
| 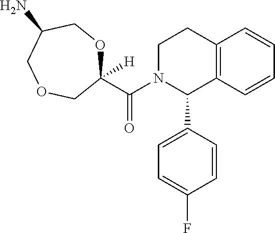 [SMILES: N[C@@H]1COC[C@H](C(=O)N2CCc3ccccc3[C@@H]2c2ccc(F)cc2)OC1] | 5401 |
| 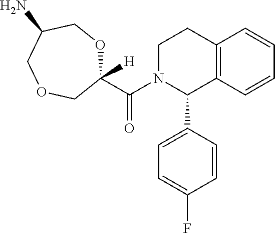 [SMILES: N[C@@H]1COC[C@@H](C(=O)N2CCc3ccccc3[C@@H]2c2ccc(F)cc2)OC1] | 5402 |
| 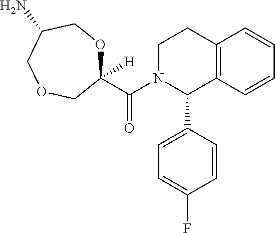 [SMILES: N[C@H]1COC[C@H](C(=O)N2CCc3ccccc3[C@@H]2c2ccc(F)cc2)OC1] | 5403 |
| 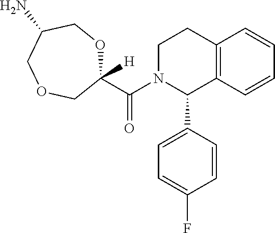 [SMILES: N[C@H]1COC[C@@H](C(=O)N2CCc3ccccc3[C@@H]2c2ccc(F)cc2)OC1] | 5404 |
| 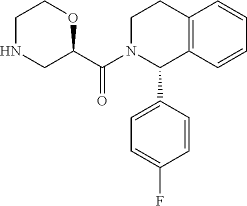 [SMILES: O=C([C@H]1CNCCO1)N1CCc2ccccc2[C@@H]1c1ccc(F)cc1] | 5405 |
| 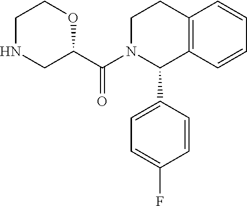 [SMILES: O=C([C@@H]1CNCCO1)N1CCc2ccccc2[C@@H]1c1ccc(F)cc1] | 5406 |
| 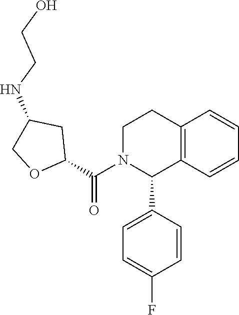 [SMILES: O=C([C@H]1C[C@@H](NCCO)CO1)N1CCc2ccccc2[C@@H]1c1ccc(F)cc1] | 5407 |
| 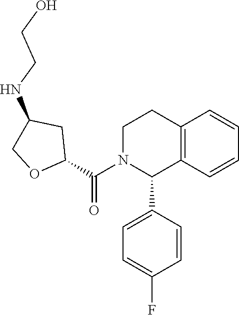 [SMILES: O=C([C@H]1C[C@H](NCCO)CO1)N1CCc2ccccc2[C@@H]1c1ccc(F)cc1] | 5408 |
| 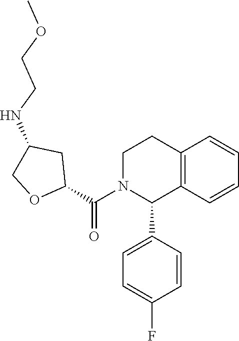 [SMILES: COCCN[C@H]1CO[C@@H](C(=O)N2CCc3ccccc3[C@@H]2c2ccc(F)cc2)C1] | 5409 |
| 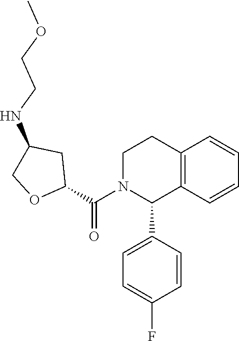 [SMILES: COCCN[C@@H]1CO[C@@H](C(=O)N2CCc3ccccc3[C@@H]2c2ccc(F)cc2)C1] | 5410 |
| 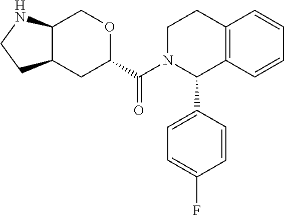 [SMILES: O=C([C@@H]1C[C@H]2CCN[C@H]2CO1)N1CCc2ccccc2[C@@H]1c1ccc(F)cc1] | 5411 |
| 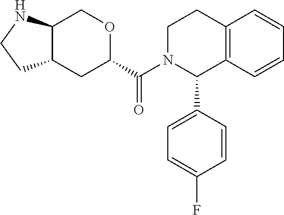 [SMILES: O=C([C@@H]1C[C@@H]2CCN[C@H]2CO1)N1CCc2ccccc2[C@@H]1c1ccc(F)cc1] | 5412 |
| 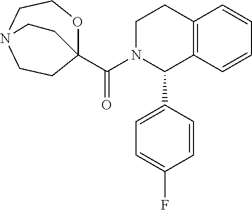 [SMILES: O=C(N1CCc2ccccc2[C@@H]1c1ccc(F)cc1)C12CCN(CCO1)CC2] | 5413 |
| 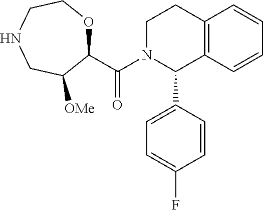 [SMILES: CO[C@H]1CNCCO[C@H]1C(=O)N1CCc2ccccc2[C@@H]1c1ccc(F)cc1] | 5414 |
| 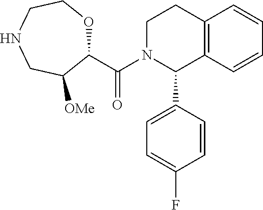 [SMILES: CO[C@H]1CNCCO[C@@H]1C(=O)N1CCc2ccccc2[C@@H]1c1ccc(F)cc1] | 5415 |
| 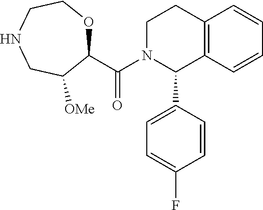 [SMILES: CO[C@@H]1CNCCO[C@H]1C(=O)N1CCc2ccccc2[C@@H]1c1ccc(F)cc1] | 5416 |
| 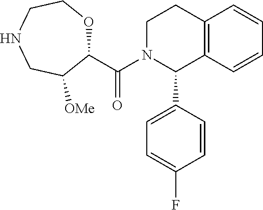 [SMILES: CO[C@@H]1CNCCO[C@@H]1C(=O)N1CCc2ccccc2[C@@H]1c1ccc(F)cc1] | 5417 |
| 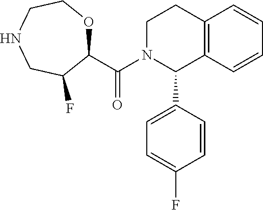 [SMILES: O=C([C@@H]1OCCNC[C@@H]1F)N1CCc2ccccc2[C@@H]1c1ccc(F)cc1] | 5418 |
| 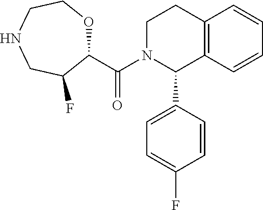 [SMILES: O=C([C@H]1OCCNC[C@@H]1F)N1CCc2ccccc2[C@@H]1c1ccc(F)cc1] | 5419 |
| 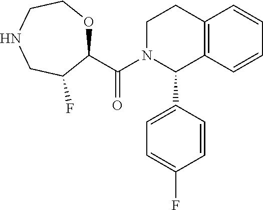 [SMILES: O=C([C@@H]1OCCNC[C@H]1F)N1CCc2ccccc2[C@@H]1c1ccc(F)cc1] | 5420 |
| 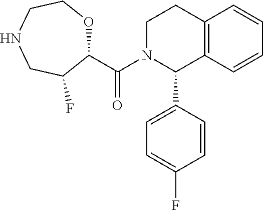 [SMILES: O=C([C@H]1OCCNC[C@H]1F)N1CCc2ccccc2[C@@H]1c1ccc(F)cc1] | 5421 |
| 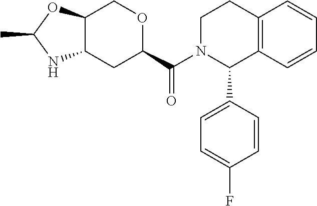 [SMILES: C[C@@H]1N[C@H]2C[C@H](C(=O)N3CCc4ccccc4[C@@H]3c3ccc(F)cc3)OC[C@@H]2O1] | 5422 |
| 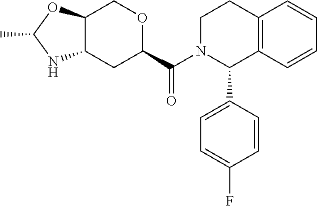 [SMILES: C[C@H]1N[C@H]2C[C@H](C(=O)N3CCc4ccccc4[C@@H]3c3ccc(F)cc3)OC[C@@H]2O1] | 5423 |
| 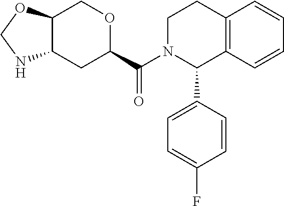 [SMILES: O=C([C@H]1C[C@@H]2NCO[C@H]2CO1)N1CCc2ccccc2[C@@H]1c1ccc(F)cc1] | 5424 |
| 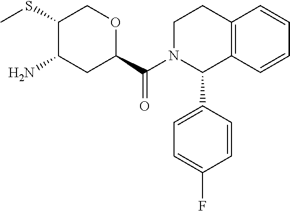 [SMILES: CS[C@@H]1CO[C@@H](C(=O)N2CCc3ccccc3[C@@H]2c2ccc(F)cc2)C[C@@H]1N] | 5425 |
| 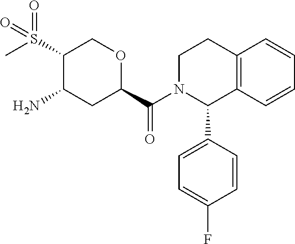 [SMILES: CS(=O)(=O)[C@@H]1CO[C@@H](C(=O)N2CCc3ccccc3[C@@H]2c2ccc(F)cc2)C[C@@H]1N] | 5426 |
| 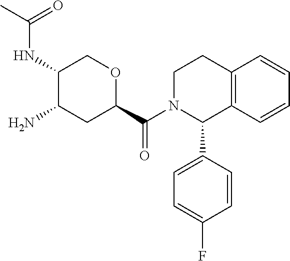 [SMILES: CC(=O)N[C@@H]1CO[C@@H](C(=O)N2CCc3ccccc3[C@@H]2c2ccc(F)cc2)C[C@@H]1N] | 5427 |
| 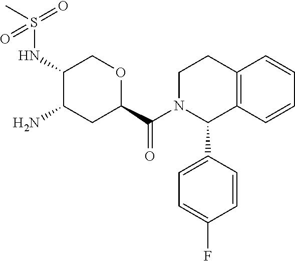 [SMILES: CS(=O)(=O)N[C@@H]1CO[C@@H](C(=O)N2CCc3ccccc3[C@@H]2c2ccc(F)cc2)C[C@@H]1N] | 5428 |
| 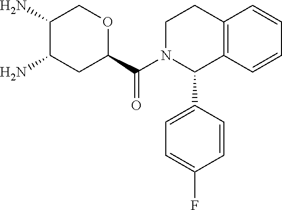 [SMILES: N[C@@H]1CO[C@@H](C(=O)N2CCc3ccccc3[C@@H]2c2ccc(F)cc2)C[C@@H]1N] | 5429 |
| 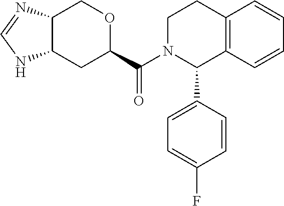 [SMILES: O=C([C@H]1C[C@@H]2NC=N[C@@H]2CO1)N1CCc2ccccc2[C@@H]1c1ccc(F)cc1] | 5430 |
| 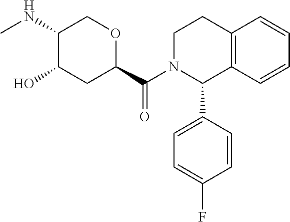 [SMILES: CN[C@@H]1CO[C@@H](C(=O)N2CCc3ccccc3[C@@H]2c2ccc(F)cc2)C[C@@H]1O] | 5431 |
| 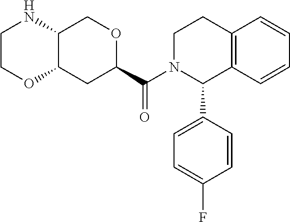 [SMILES: O=C([C@H]1C[C@@H]2OCCN[C@@H]2CO1)N1CCc2ccccc2[C@@H]1c1ccc(F)cc1] | 5432 |
| 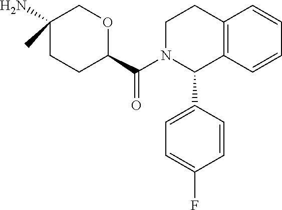 [SMILES: C[C@]1(N)CC[C@H](C(=O)N2CCc3ccccc3[C@@H]2c2ccc(F)cc2)OC1] | 5433 |
| 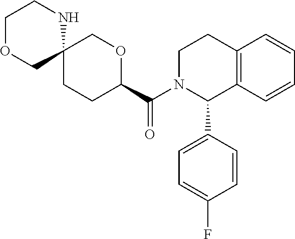 [SMILES: O=C([C@H]1CC[C@@]2(COCCN2)CO1)N1CCc2ccccc2[C@@H]1c1ccc(F)cc1] | 5434 |
| 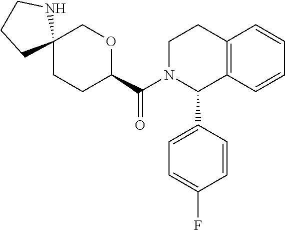 [SMILES: O=C([C@H]1CC[C@@]2(CCCN2)CO1)N1CCc2ccccc2[C@@H]1c1ccc(F)cc1] | 5435 |
| 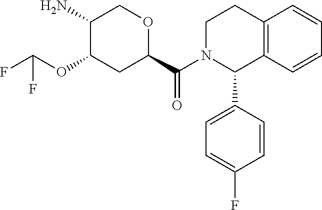 [SMILES: N[C@@H]1CO[C@@H](C(=O)N2CCc3ccccc3[C@@H]2c2ccc(F)cc2)C[C@@H]1OC(F)F] | 5436 |
| 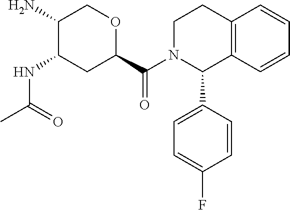 [SMILES: CC(=O)N[C@H]1C[C@H](C(=O)N2CCc3ccccc3[C@@H]2c2ccc(F)cc2)OC[C@H]1N] | 5437 |
| 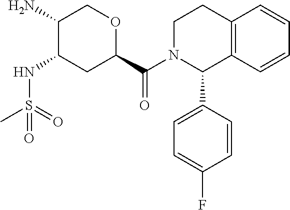 [SMILES: CS(=O)(=O)N[C@H]1C[C@H](C(=O)N2CCc3ccccc3[C@@H]2c2ccc(F)cc2)OC[C@H]1N] | 5438 |
| 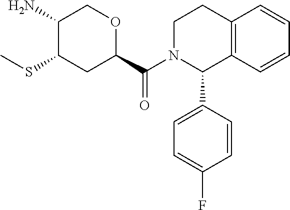 [SMILES: CS[C@H]1C[C@H](C(=O)N2CCc3ccccc3[C@@H]2c2ccc(F)cc2)OC[C@H]1N] | 5439 |
| 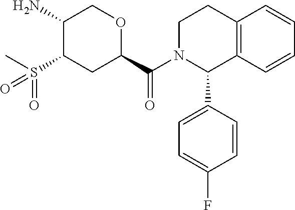 [SMILES: CS(=O)(=O)[C@H]1C[C@H](C(=O)N2CCc3ccccc3[C@@H]2c2ccc(F)cc2)OC[C@H]1N] | 5440 |
| 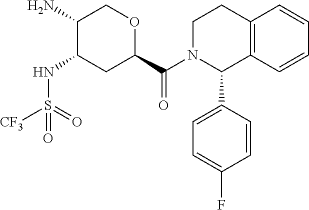 [SMILES: N[C@@H]1CO[C@@H](C(=O)N2CCc3ccccc3[C@@H]2c2ccc(F)cc2)C[C@@H]1NS(=O)(=O)C(F)(F)F] | 5441 |
| 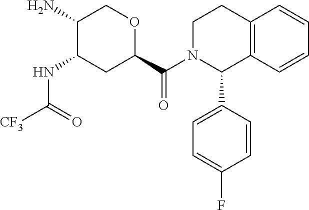 [SMILES: N[C@@H]1CO[C@@H](C(=O)N2CCc3ccccc3[C@@H]2c2ccc(F)cc2)C[C@@H]1NC(=O)C(F)(F)F] | 5442 |
| 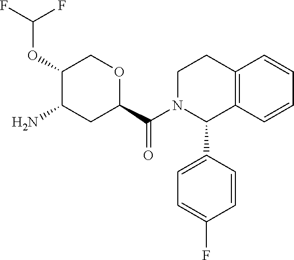 [SMILES: N[C@H]1C[C@H](C(=O)N2CCc3ccccc3[C@@H]2c2ccc(F)cc2)OC[C@H]1OC(F)F] | 5443 |
| 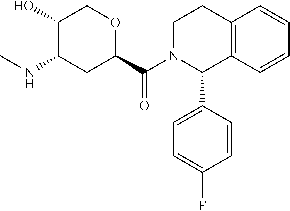 [SMILES: CN[C@H]1C[C@H](C(=O)N2CCc3ccccc3[C@@H]2c2ccc(F)cc2)OC[C@H]1O] | 5444 |
| 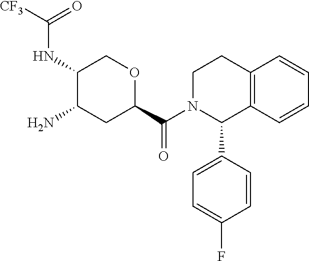 [SMILES: N[C@H]1C[C@H](C(=O)N2CCc3ccccc3[C@@H]2c2ccc(F)cc2)OC[C@H]1NC(=O)C(F)(F)F] | 5445 |
| 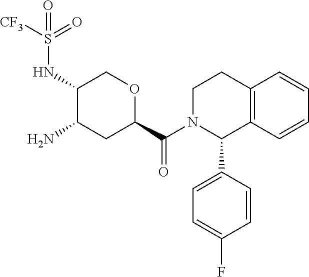 [SMILES: N[C@H]1C[C@H](C(=O)N2CCc3ccccc3[C@@H]2c2ccc(F)cc2)OC[C@H]1NS(=O)(=O)C(F)(F)F] | 5446 |
| 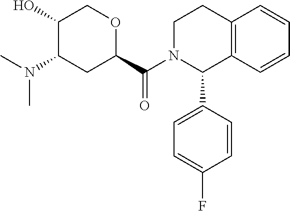 [SMILES: CN(C)[C@H]1C[C@H](C(=O)N2CCc3ccccc3[C@@H]2c2ccc(F)cc2)OC[C@H]1O] | 5447 |
| 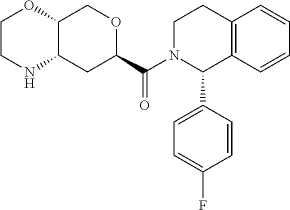 [SMILES: O=C([C@H]1C[C@@H]2NCCO[C@@H]2CO1)N1CCc2ccccc2[C@@H]1c1ccc(F)cc1] | 5448 |
| 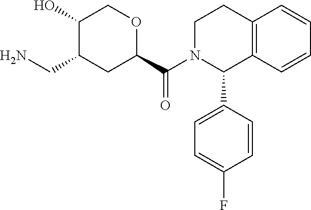 [SMILES: NC[C@H]1C[C@H](C(=O)N2CCc3ccccc3[C@@H]2c2ccc(F)cc2)OC[C@H]1O] | 5449 |
| 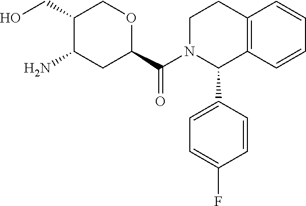 [SMILES: N[C@H]1C[C@H](C(=O)N2CCc3ccccc3[C@@H]2c2ccc(F)cc2)OC[C@H]1CO] | 5450 |
| 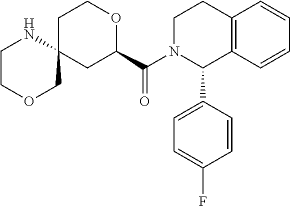 [SMILES: O=C([C@H]1C[C@]2(CCO1)COCCN2)N1CCc2ccccc2[C@@H]1c1ccc(F)cc1] | 5451 |
| 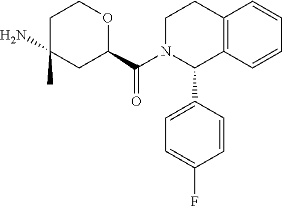 [SMILES: C[C@@]1(N)CCO[C@@H](C(=O)N2CCc3ccccc3[C@@H]2c2ccc(F)cc2)C1] | 5452 |
| 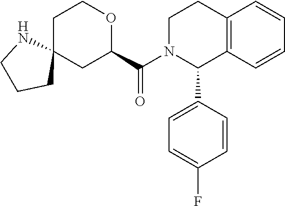 [SMILES: O=C([C@H]1C[C@@]2(CCCN2)CCO1)N1CCc2ccccc2[C@@H]1c1ccc(F)cc1] | 5453 |
| 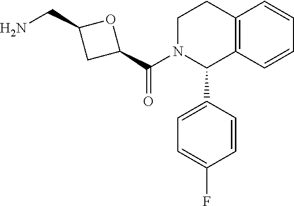 [SMILES: NC[C@@H]1C[C@H](C(=O)N2CCc3ccccc3[C@@H]2c2ccc(F)cc2)O1] | 5454 |
| 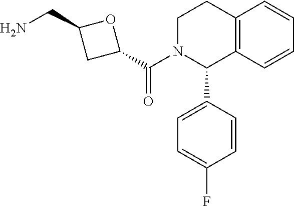 [SMILES: NC[C@@H]1C[C@@H](C(=O)N2CCc3ccccc3[C@@H]2c2ccc(F)cc2)O1] | 5455 |
| 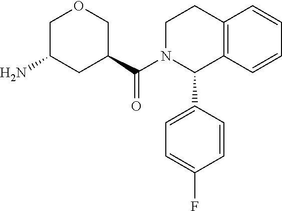 [SMILES: N[C@@H]1COC[C@@H](C(=O)N2CCc3ccccc3[C@@H]2c2ccc(F)cc2)C1] | 5456 |
| 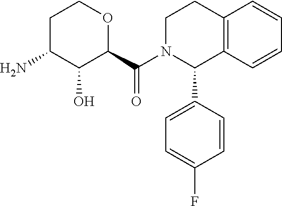 [SMILES: N[C@@H]1CCO[C@@H](C(=O)N2CCc3ccccc3[C@@H]2c2ccc(F)cc2)[C@@H]1O] | 5457 |
| 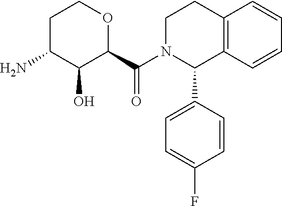 [SMILES: N[C@@H]1CCO[C@@H](C(=O)N2CCc3ccccc3[C@@H]2c2ccc(F)cc2)[C@H]1O] | 5458 |
| 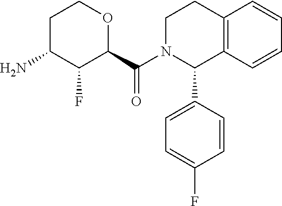 [SMILES: N[C@@H]1CCO[C@@H](C(=O)N2CCc3ccccc3[C@@H]2c2ccc(F)cc2)[C@@H]1F] | 5459 |
| 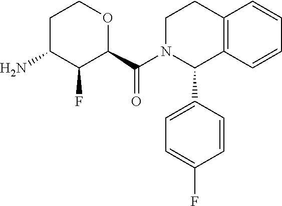 [SMILES: N[C@@H]1CCO[C@@H](C(=O)N2CCc3ccccc3[C@@H]2c2ccc(F)cc2)[C@H]1F] | 5460 |
| 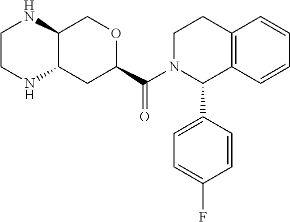 [SMILES: O=C([C@H]1C[C@@H]2NCCN[C@H]2CO1)N1CCc2ccccc2[C@@H]1c1ccc(F)cc1] | 5461 |
| 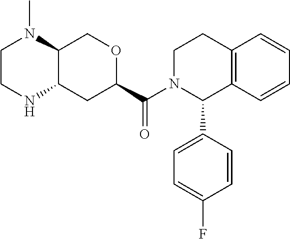 [SMILES: CN1CCN[C@H]2C[C@H](C(=O)N3CCc4ccccc4[C@@H]3c3ccc(F)cc3)OC[C@@H]21] | 5462 |
| 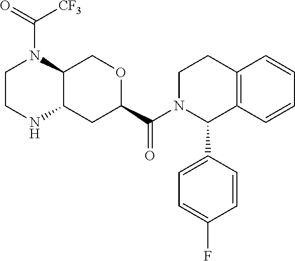 [SMILES: O=C([C@H]1C[C@@H]2NCCN(C(=O)C(F)(F)F)[C@H]2CO1)N1CCc2ccccc2[C@@H]1c1ccc(F)cc1] | 5463 |
| 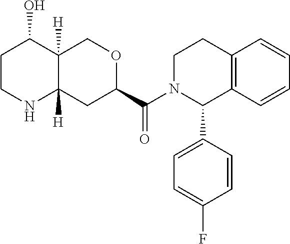 [SMILES: O=C([C@H]1C[C@@H]2NCC[C@H](O)[C@H]2CO1)N1CCc2ccccc2[C@@H]1c1ccc(F)cc1] | 5464 |
| 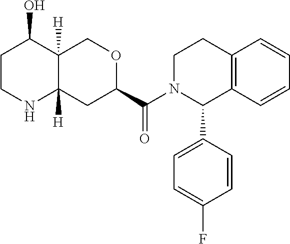 [SMILES: O=C([C@H]1C[C@@H]2NCC[C@@H](O)[C@H]2CO1)N1CCc2ccccc2[C@@H]1c1ccc(F)cc1] | 5465 |
| 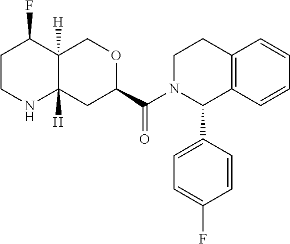 [SMILES: O=C([C@H]1C[C@@H]2NCC[C@@H](F)[C@H]2CO1)N1CCc2ccccc2[C@@H]1c1ccc(F)cc1] | 5466 |
| 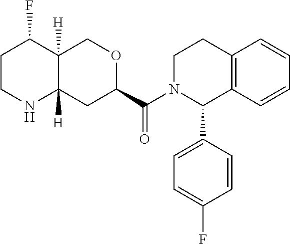 [SMILES: O=C([C@H]1C[C@@H]2NCC[C@H](F)[C@H]2CO1)N1CCc2ccccc2[C@@H]1c1ccc(F)cc1] | 5467 |
| 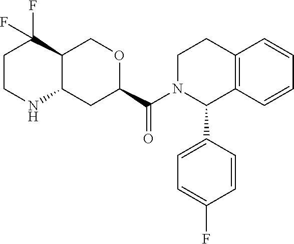 [SMILES: O=C([C@H]1C[C@@H]2NCCC(F)(F)[C@H]2CO1)N1CCc2ccccc2[C@@H]1c1ccc(F)cc1] | 5468 |
| 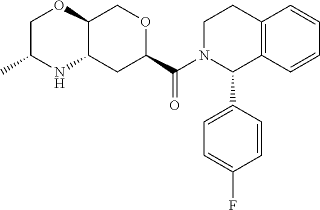 [SMILES: C[C@@H]1CO[C@H]2CO[C@@H](C(=O)N3CCc4ccccc4[C@@H]3c3ccc(F)cc3)C[C@@H]2N1] | 5469 |
| 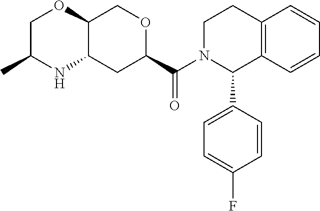 [SMILES: C[C@H]1CO[C@H]2CO[C@@H](C(=O)N3CCc4ccccc4[C@@H]3c3ccc(F)cc3)C[C@@H]2N1] | 5470 |
| 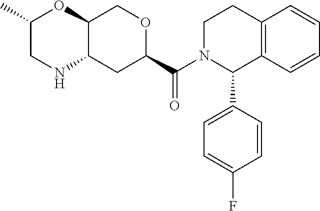 [SMILES: C[C@H]1CN[C@H]2C[C@H](C(=O)N3CCc4ccccc4[C@@H]3c3ccc(F)cc3)OC[C@@H]2O1] | 5471 |
| 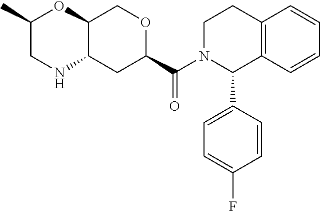 [SMILES: C[C@@H]1CN[C@H]2C[C@H](C(=O)N3CCc4ccccc4[C@@H]3c3ccc(F)cc3)OC[C@@H]2O1] | 5472 |
| 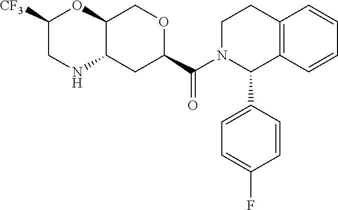 [SMILES: O=C([C@H]1C[C@@H]2NC[C@@H](C(F)(F)F)O[C@H]2CO1)N1CCc2ccccc2[C@@H]1c1ccc(F)cc1] | 5473 |
| 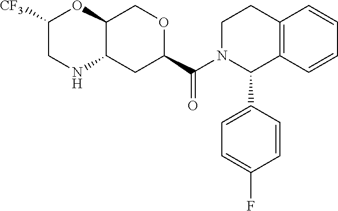 [SMILES: O=C([C@H]1C[C@@H]2NC[C@H](C(F)(F)F)O[C@H]2CO1)N1CCc2ccccc2[C@@H]1c1ccc(F)cc1] | 5474 |
| 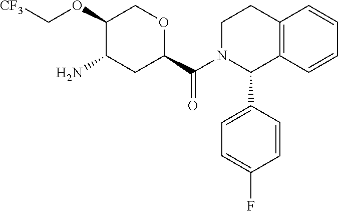 [SMILES: N[C@H]1C[C@H](C(=O)N2CCc3ccccc3[C@@H]2c2ccc(F)cc2)OC[C@@H]1OCC(F)(F)F] | 5475 |
| 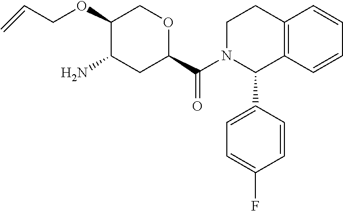 [SMILES: C=CCO[C@H]1CO[C@@H](C(=O)N2CCc3ccccc3[C@@H]2c2ccc(F)cc2)C[C@@H]1N] | 5476 |
| 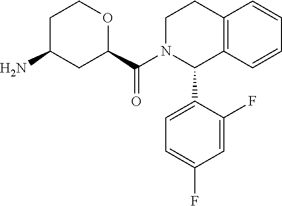 [SMILES: N[C@H]1CCO[C@@H](C(=O)N2CCc3ccccc3[C@@H]2c2ccc(F)cc2F)C1] | 5500 |
| 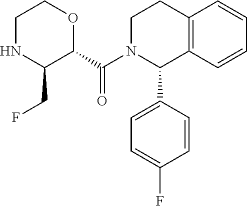 [SMILES: O=C([C@H]1OCCN[C@@H]1CF)N1CCc2ccccc2[C@@H]1c1ccc(F)cc1] | 5501 |
| 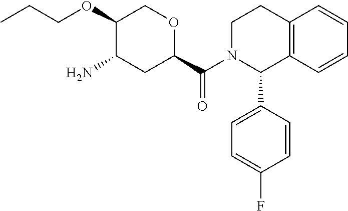 [SMILES: CCCO[C@H]1CO[C@@H](C(=O)N2CCc3ccccc3[C@@H]2c2ccc(F)cc2)C[C@@H]1N] | 5502 |
| 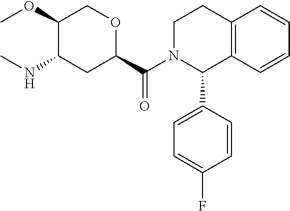 [SMILES: CN[C@H]1C[C@H](C(=O)N2CCc3ccccc3[C@@H]2c2ccc(F)cc2)OC[C@@H]1OC] | 5503 |
| 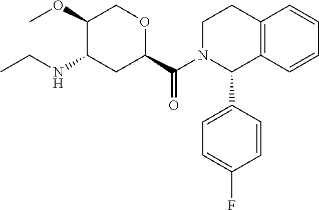 [SMILES: CCN[C@H]1C[C@H](C(=O)N2CCc3ccccc3[C@@H]2c2ccc(F)cc2)OC[C@@H]1OC] | 5504 |
| 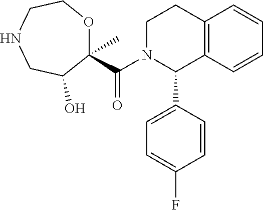 [SMILES: C[C@@]1(C(=O)N2CCc3ccccc3[C@@H]2c2ccc(F)cc2)OCCNC[C@H]1O] | 5505 |
| 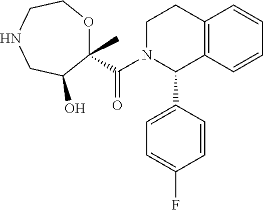 [SMILES: C[C@]1(C(=O)N2CCc3ccccc3[C@@H]2c2ccc(F)cc2)OCCNC[C@@H]1O] | 5506 |
| 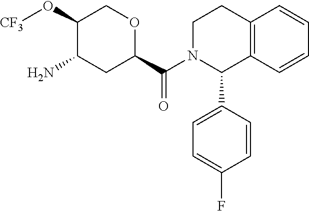 [SMILES: N[C@H]1C[C@H](C(=O)N2CCc3ccccc3[C@@H]2c2ccc(F)cc2)OC[C@@H]1OC(F)(F)F] | 5507 |
| 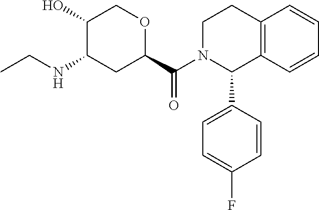 [SMILES: CCN[C@H]1C[C@H](C(=O)N2CCc3ccccc3[C@@H]2c2ccc(F)cc2)OC[C@H]1O] | 5508 |
| 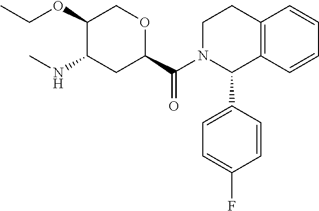 [SMILES: CCO[C@H]1CO[C@@H](C(=O)N2CCc3ccccc3[C@@H]2c2ccc(F)cc2)C[C@@H]1NC] | 5509 |
| 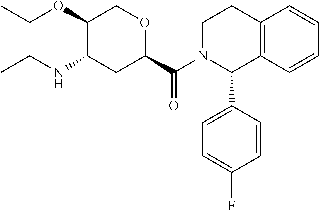 [SMILES: CCN[C@H]1C[C@H](C(=O)N2CCc3ccccc3[C@@H]2c2ccc(F)cc2)OC[C@@H]1OCC] | 5510 |
| 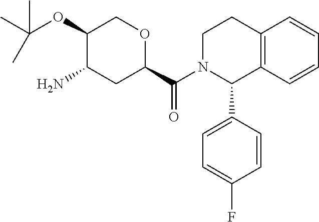 [SMILES: CC(C)(C)O[C@H]1CO[C@@H](C(=O)N2CCc3ccccc3[C@@H]2c2ccc(F)cc2)C[C@@H]1N] | 5511 |
| 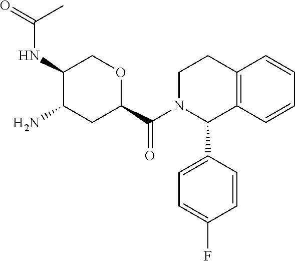 [SMILES: CC(=O)N[C@H]1CO[C@@H](C(=O)N2CCc3ccccc3[C@@H]2c2ccc(F)cc2)C[C@@H]1N] | 5512 |
| 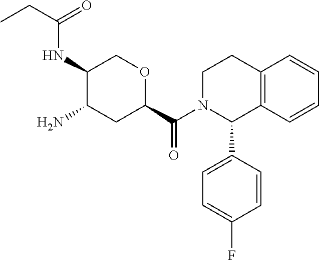 [SMILES: CCC(=O)N[C@H]1CO[C@@H](C(=O)N2CCc3ccccc3[C@@H]2c2ccc(F)cc2)C[C@@H]1N] | 5513 |
| 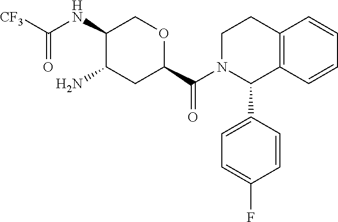 [SMILES: N[C@H]1C[C@H](C(=O)N2CCc3ccccc3[C@@H]2c2ccc(F)cc2)OC[C@@H]1NC(=O)C(F)(F)F] | 5514 |
| 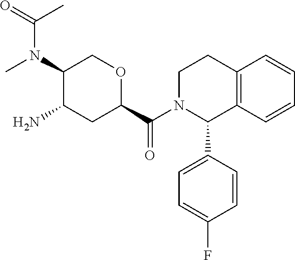 [SMILES: CC(=O)N(C)[C@H]1CO[C@@H](C(=O)N2CCc3ccccc3[C@@H]2c2ccc(F)cc2)C[C@@H]1N] | 5516 |
| 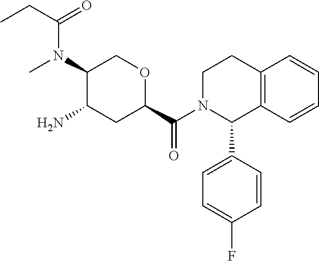 [SMILES: CCC(=O)N(C)[C@H]1CO[C@@H](C(=O)N2CCc3ccccc3[C@@H]2c2ccc(F)cc2)C[C@@H]1N] | 5517 |
| 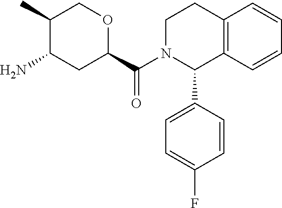 [SMILES: C[C@H]1CO[C@@H](C(=O)N2CCc3ccccc3[C@@H]2c2ccc(F)cc2)C[C@@H]1N] | 5518 |
| 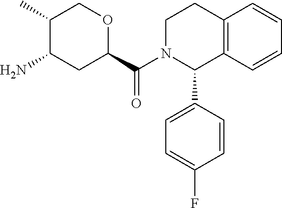 [SMILES: C[C@@H]1CO[C@@H](C(=O)N2CCc3ccccc3[C@@H]2c2ccc(F)cc2)C[C@@H]1N] | 5519 |
| 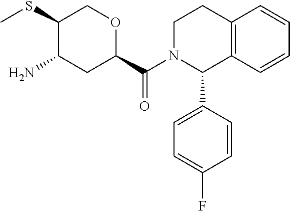 [SMILES: CS[C@H]1CO[C@@H](C(=O)N2CCc3ccccc3[C@@H]2c2ccc(F)cc2)C[C@@H]1N] | 5520 |
| 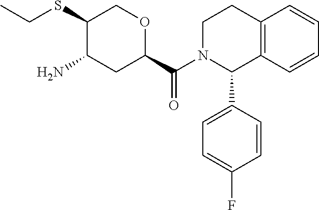 [SMILES: CCS[C@H]1CO[C@@H](C(=O)N2CCc3ccccc3[C@@H]2c2ccc(F)cc2)C[C@@H]1N] | 5521 |
| 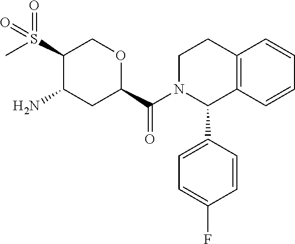 [SMILES: CS(=O)(=O)[C@H]1CO[C@@H](C(=O)N2CCc3ccccc3[C@@H]2c2ccc(F)cc2)C[C@@H]1N] | 5523 |
| 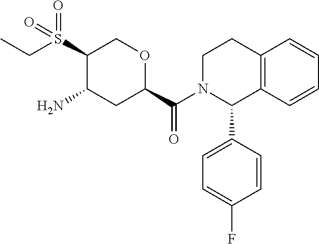 [SMILES: CCS(=O)(=O)[C@H]1CO[C@@H](C(=O)N2CCc3ccccc3[C@@H]2c2ccc(F)cc2)C[C@@H]1N] | 5524 |
| 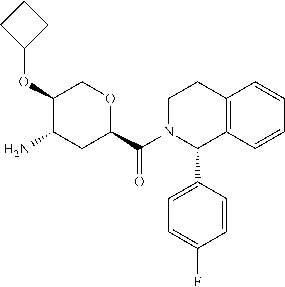 [SMILES: N[C@H]1C[C@H](C(=O)N2CCc3ccccc3[C@@H]2c2ccc(F)cc2)OC[C@@H]1OC1CCC1] | 5525 |
| 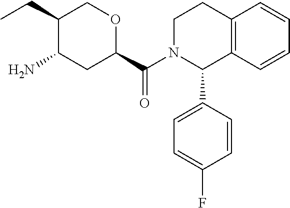 [SMILES: CC[C@H]1CO[C@@H](C(=O)N2CCc3ccccc3[C@@H]2c2ccc(F)cc2)C[C@@H]1N] | 5526 |
| 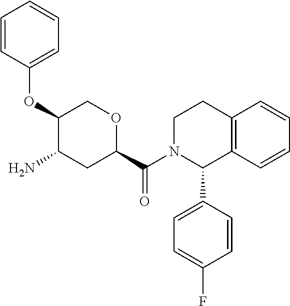 [SMILES: N[C@H]1C[C@H](C(=O)N2CCc3ccccc3[C@@H]2c2ccc(F)cc2)OC[C@@H]1Oc1ccccc1] | 5527 |
| 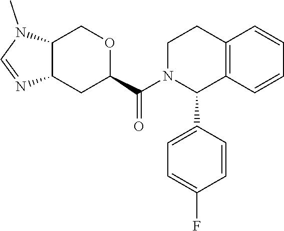 [SMILES: CN1C=N[C@H]2C[C@H](C(=O)N3CCc4ccccc4[C@@H]3c3ccc(F)cc3)OC[C@H]21] | 5528 |
| 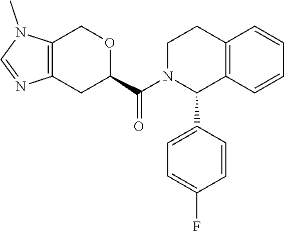 [SMILES: Cn1cnc2c1CO[C@@H](C(=O)N1CCc3ccccc3[C@@H]1c1ccc(F)cc1)C2] | 5529 |
| 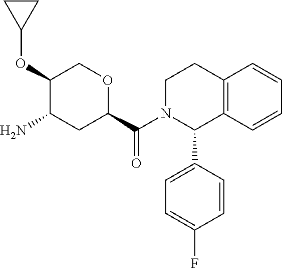 [SMILES: N[C@H]1C[C@H](C(=O)N2CCc3ccccc3[C@@H]2c2ccc(F)cc2)OC[C@@H]1OC1CC1] | 5530 |
| 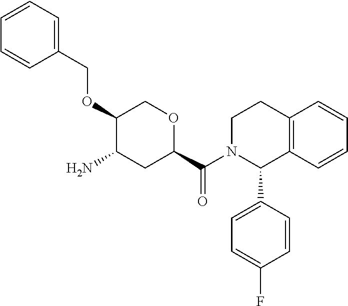 [SMILES: N[C@H]1C[C@H](C(=O)N2CCc3ccccc3[C@@H]2c2ccc(F)cc2)OC[C@@H]1OCc1ccccc1] | 5531 |
| 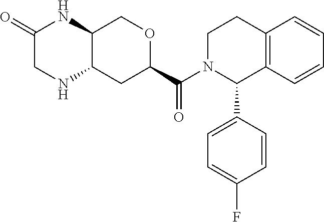 [SMILES: O=C1CN[C@H]2C[C@H](C(=O)N3CCc4ccccc4[C@@H]3c3ccc(F)cc3)OC[C@@H]2N1] | 5532 |
| 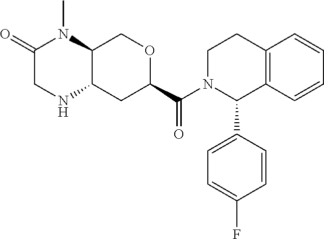 [SMILES: CN1C(=O)CN[C@H]2C[C@H](C(=O)N3CCc4ccccc4[C@@H]3c3ccc(F)cc3)OC[C@@H]21] | 5533 |
| 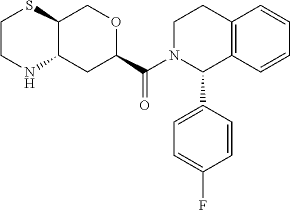 [SMILES: O=C([C@H]1C[C@@H]2NCCS[C@H]2CO1)N1CCc2ccccc2[C@@H]1c1ccc(F)cc1] | 5534 |
| 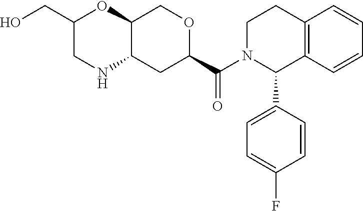 | 5535 |
| 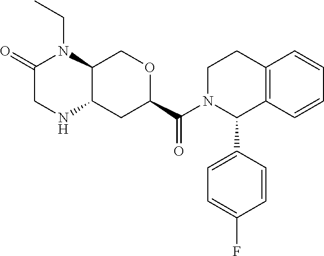 [SMILES: CCN1C(=O)CN[C@H]2C[C@H](C(=O)N3CCc4ccccc4[C@@H]3c3ccc(F)cc3)OC[C@@H]21] | 5536 |
| 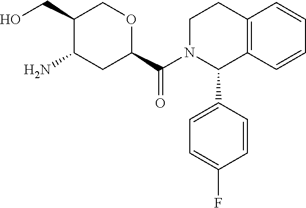 [SMILES: N[C@H]1C[C@H](C(=O)N2CCc3ccccc3[C@@H]2c2ccc(F)cc2)OC[C@@H]1CO] | 5537 |
| 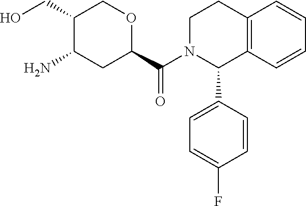 [SMILES: N[C@H]1C[C@H](C(=O)N2CCc3ccccc3[C@@H]2c2ccc(F)cc2)OC[C@H]1CO] | 5538 |
| 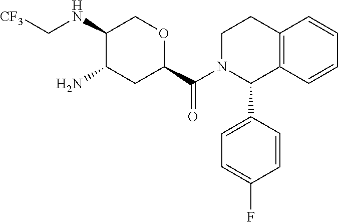 [SMILES: N[C@H]1C[C@H](C(=O)N2CCc3ccccc3[C@@H]2c2ccc(F)cc2)OC[C@@H]1NCC(F)(F)F] | 5539 |
| 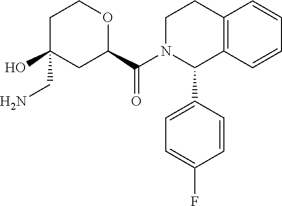 [SMILES: NC[C@]1(O)CCO[C@@H](C(=O)N2CCc3ccccc3[C@@H]2c2ccc(F)cc2)C1] | 5540 |
| 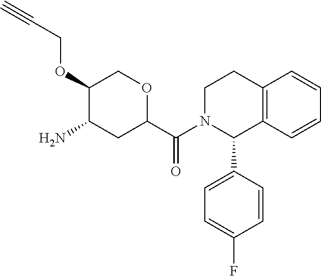 [SMILES: C#CCO[C@H]1COC(C(=O)N2CCc3ccccc3[C@@H]2c2ccc(F)cc2)C[C@@H]1N] | 5541 |
| 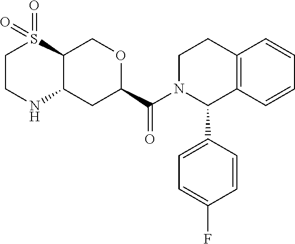 [SMILES: O=C([C@H]1C[C@@H]2NCCS(=O)(=O)[C@H]2CO1)N1CCc2ccccc2[C@@H]1c1ccc(F)cc1] | 5542 |
| 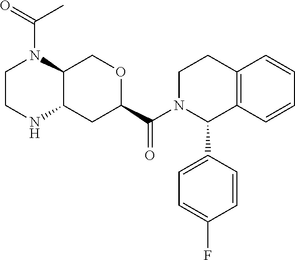 [SMILES: CC(=O)N1CCN[C@H]2C[C@H](C(=O)N3CCc4ccccc4[C@@H]3c3ccc(F)cc3)OC[C@@H]21] | 5543 |
| 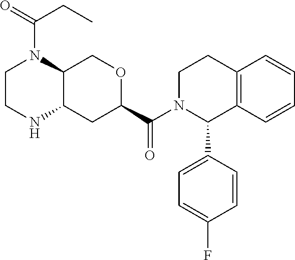 [SMILES: CCC(=O)N1CCN[C@H]2C[C@H](C(=O)N3CCc4ccccc4[C@@H]3c3ccc(F)cc3)OC[C@@H]21] | 5544 |
| 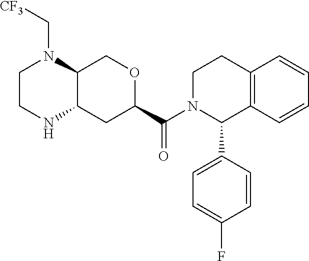 [SMILES: O=C([C@H]1C[C@@H]2NCCN(CC(F)(F)F)[C@H]2CO1)N1CCc2ccccc2[C@@H]1c1ccc(F)cc1] | 5545 |
| 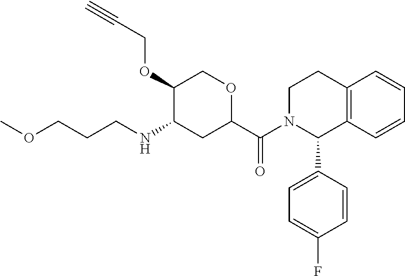 [SMILES: C#CCO[C@H]1COC(C(=O)N2CCc3ccccc3[C@@H]2c2ccc(F)cc2)C[C@@H]1NCCCOC] | 5546 |
| 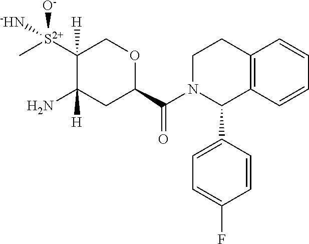 [SMILES: C[S@@+2]([NH-])([O-])[C@H]1CO[C@@H](C(=O)N2CCc3ccccc3[C@@H]2c2ccc(F)cc2)C[C@@H]1N] | 5547 |
| 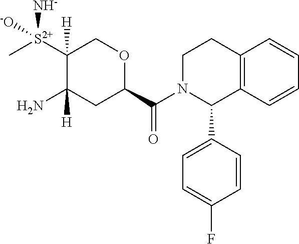 | 5548 |
| 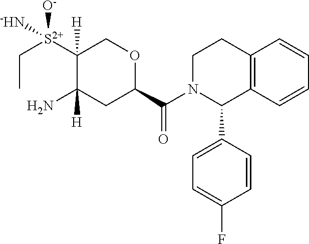 [SMILES: CC[S@@+2]([NH-])([O-])[C@H]1CO[C@@H](C(=O)N2CCc3ccccc3[C@@H]2c2ccc(F)cc2)C[C@@H]1N] | 5549 |
| 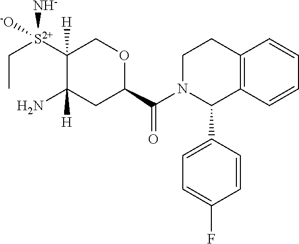 | 5550 |
| 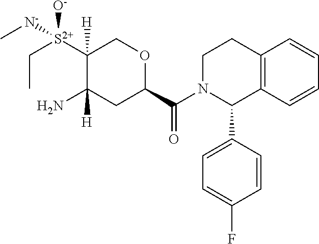 [SMILES: CC[S@+2]([O-])([N-]C)[C@H]1CO[C@@H](C(=O)N2CCc3ccccc3[C@@H]2c2ccc(F)cc2)C[C@@H]1N] | 5551 |
| 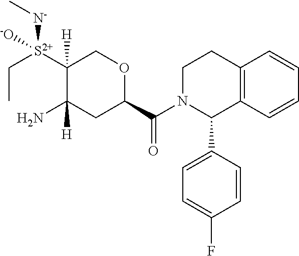 | 5552 |
| 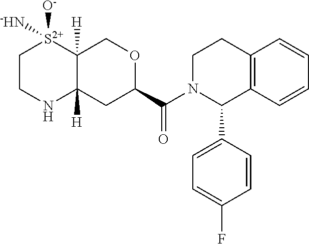 [SMILES: [NH-][S@@+2]1([O-])CCN[C@H]2C[C@H](C(=O)N3CCc4ccccc4[C@@H]3c3ccc(F)cc3)OC[C@@H]21] | 5553 |
| 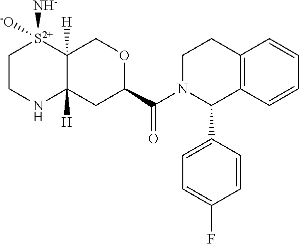 | 5554 |
| 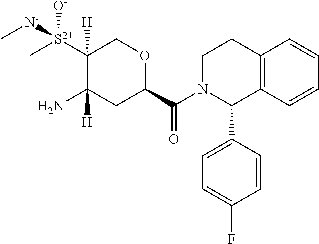 [SMILES: C[N-][S@@+2](C)([O-])[C@H]1CO[C@@H](C(=O)N2CCc3ccccc3[C@@H]2c2ccc(F)cc2)C[C@@H]1N] | 5555 |
| 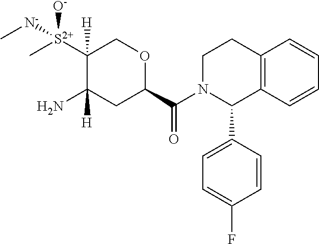 [SMILES: C[N-][S@+2](C)([O-])[C@H]1CO[C@@H](C(=O)N2CCc3ccccc3[C@@H]2c2ccc(F)cc2)C[C@@H]1N] | 5556 |
| 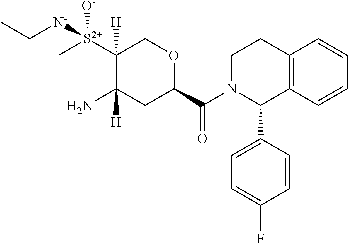 [SMILES: CC[N-][S@@+2](C)([O-])[C@H]1CO[C@@H](C(=O)N2CCc3ccccc3[C@@H]2c2ccc(F)cc2)C[C@@H]1N] | 5557 |
| 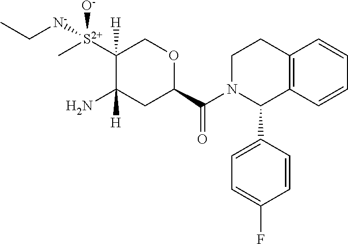 [SMILES: CC[N-][S@+2](C)([O-])[C@H]1CO[C@@H](C(=O)N2CCc3ccccc3[C@@H]2c2ccc(F)cc2)C[C@@H]1N] | 5558 |
| 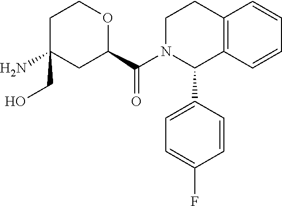 [SMILES: N[C@]1(CO)CCO[C@@H](C(=O)N2CCc3ccccc3[C@@H]2c2ccc(F)cc2)C1] | 5559 |
| 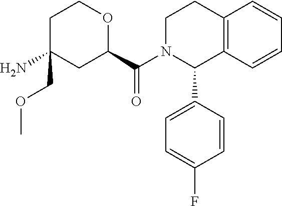 [SMILES: COC[C@@]1(N)CCO[C@@H](C(=O)N2CCc3ccccc3[C@@H]2c2ccc(F)cc2)C1] | 5560 |
| 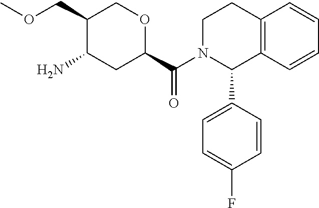 [SMILES: COC[C@H]1CO[C@@H](C(=O)N2CCc3ccccc3[C@@H]2c2ccc(F)cc2)C[C@@H]1N] | 5561 |
| 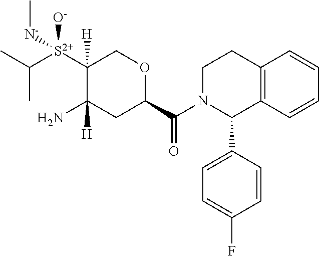 [SMILES: C[N-][S@@+2]([O-])(C(C)C)[C@H]1CO[C@@H](C(=O)N2CCc3ccccc3[C@@H]2c2ccc(F)cc2)C[C@@H]1N] | 5562 |
| 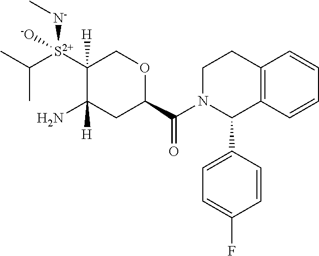 | 5563 |
| 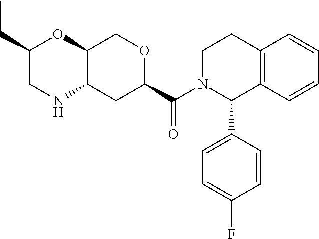 [SMILES: CC[C@@H]1CN[C@H]2C[C@H](C(=O)N3CCc4ccccc4[C@@H]3c3ccc(F)cc3)OC[C@@H]2O1] | 5564 |
| 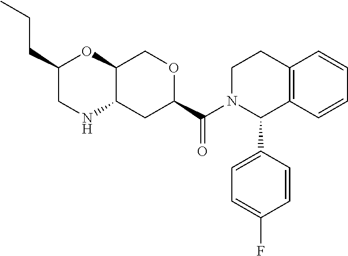 [SMILES: CCC[C@@H]1CN[C@H]2C[C@H](C(=O)N3CCc4ccccc4[C@@H]3c3ccc(F)cc3)OC[C@@H]2O1] | 5565 |
| 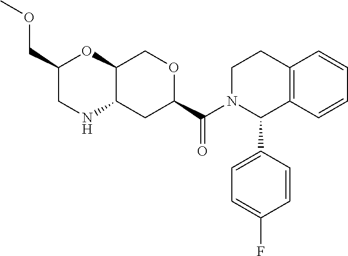 [SMILES: COC[C@@H]1CN[C@H]2C[C@H](C(=O)N3CCc4ccccc4[C@@H]3c3ccc(F)cc3)OC[C@@H]2O1] | 5566 |
| 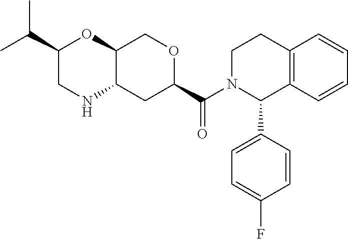 [SMILES: CC(C)[C@@H]1CN[C@H]2C[C@H](C(=O)N3CCc4ccccc4[C@@H]3c3ccc(F)cc3)OC[C@@H]2O1] | 5567 |
| 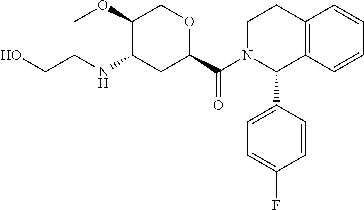 [SMILES: CO[C@H]1CO[C@@H](C(=O)N2CCc3ccccc3[C@@H]2c2ccc(F)cc2)C[C@@H]1NCCO] | 5568 |
| 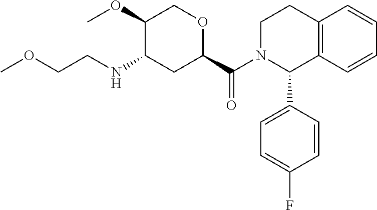 [SMILES: COCCN[C@H]1C[C@H](C(=O)N2CCc3ccccc3[C@@H]2c2ccc(F)cc2)OC[C@@H]1OC] | 5569 |
| 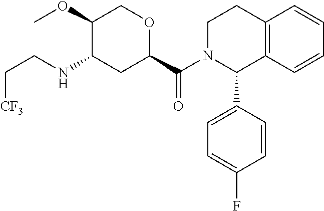 [SMILES: CO[C@H]1CO[C@@H](C(=O)N2CCc3ccccc3[C@@H]2c2ccc(F)cc2)C[C@@H]1NCCC(F)(F)F] | 5570 |
| 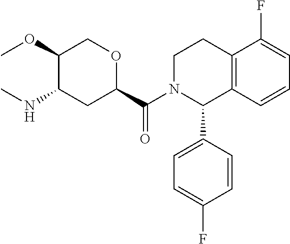 [SMILES: CN[C@H]1C[C@H](C(=O)N2CCc3c(F)cccc3[C@@H]2c2ccc(F)cc2)OC[C@@H]1OC] | 5571 |
| 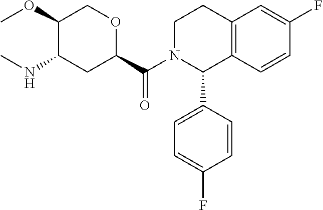 [SMILES: CN[C@H]1C[C@H](C(=O)N2CCc3cc(F)ccc3[C@@H]2c2ccc(F)cc2)OC[C@@H]1OC] | 5572 |
| 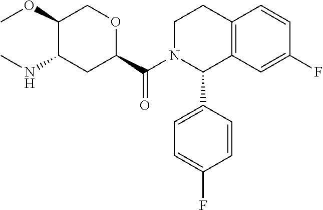 [SMILES: CN[C@H]1C[C@H](C(=O)N2CCc3ccc(F)cc3[C@@H]2c2ccc(F)cc2)OC[C@@H]1OC] | 5573 |
| 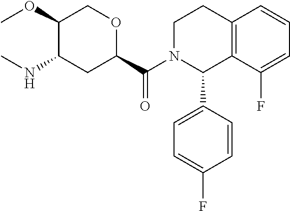 [SMILES: CN[C@H]1C[C@H](C(=O)N2CCc3cccc(F)c3[C@@H]2c2ccc(F)cc2)OC[C@@H]1OC] | 5574 |
| 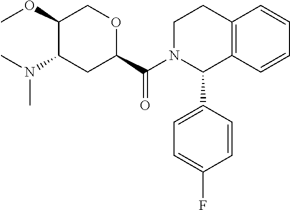 [SMILES: CO[C@H]1CO[C@@H](C(=O)N2CCc3ccccc3[C@@H]2c2ccc(F)cc2)C[C@@H]1N(C)C] | 5575 |
| 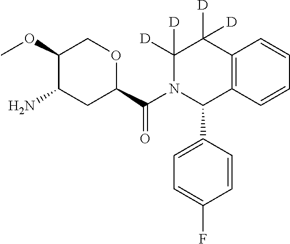 [SMILES: [2H]C1([2H])c2ccccc2[C@H](c2ccc(F)cc2)N(C(=O)[C@H]2C[C@H](N)[C@@H](OC)CO2)C1([2H])[2H]] | 5576 |
| 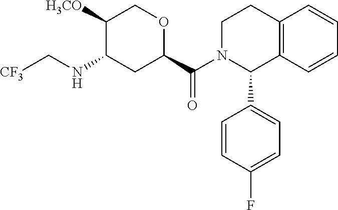 [SMILES: CO[C@H]1CO[C@@H](C(=O)N2CCc3ccccc3[C@@H]2c2ccc(F)cc2)C[C@@H]1NCC(F)(F)F] | 5577 |
| 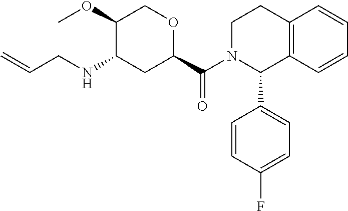 [SMILES: C=CCN[C@H]1C[C@H](C(=O)N2CCc3ccccc3[C@@H]2c2ccc(F)cc2)OC[C@@H]1OC] | 5578 |
| 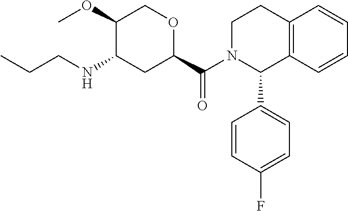 [SMILES: CCCN[C@H]1C[C@H](C(=O)N2CCc3ccccc3[C@@H]2c2ccc(F)cc2)OC[C@@H]1OC] | 5579 |
| 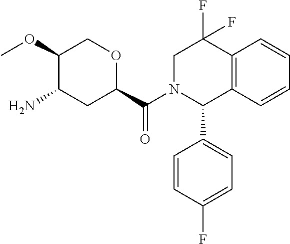 [SMILES: CO[C@H]1CO[C@@H](C(=O)N2CC(F)(F)c3ccccc3[C@@H]2c2ccc(F)cc2)C[C@@H]1N] | 5580 |
| 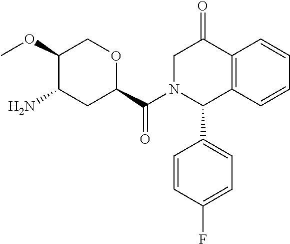 [SMILES: CO[C@H]1CO[C@@H](C(=O)N2CC(=O)c3ccccc3[C@@H]2c2ccc(F)cc2)C[C@@H]1N] | 5581 |
| 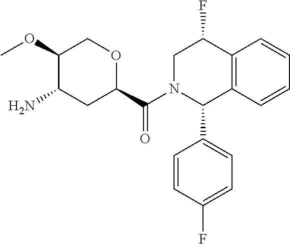 [SMILES: CO[C@H]1CO[C@@H](C(=O)N2C[C@H](F)c3ccccc3[C@@H]2c2ccc(F)cc2)C[C@@H]1N] | 5582 |
| 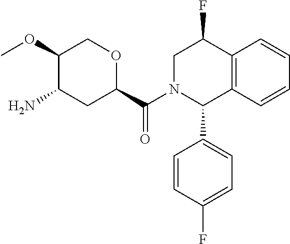 [SMILES: CO[C@H]1CO[C@@H](C(=O)N2C[C@@H](F)c3ccccc3[C@@H]2c2ccc(F)cc2)C[C@@H]1N] | 5583 |
| 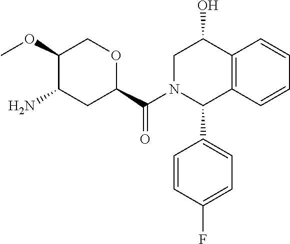 [SMILES: CO[C@H]1CO[C@@H](C(=O)N2C[C@H](O)c3ccccc3[C@@H]2c2ccc(F)cc2)C[C@@H]1N] | 5584 |
| 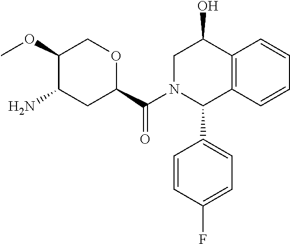 [SMILES: CO[C@H]1CO[C@@H](C(=O)N2C[C@@H](O)c3ccccc3[C@@H]2c2ccc(F)cc2)C[C@@H]1N] | 5585 |
| 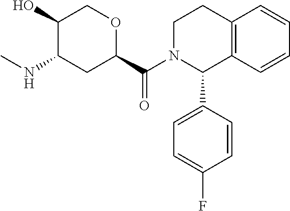 [SMILES: CN[C@H]1C[C@H](C(=O)N2CCc3ccccc3[C@@H]2c2ccc(F)cc2)OC[C@@H]1O] | 5586 |
| 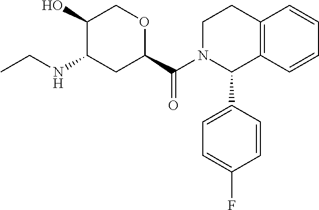 [SMILES: CCN[C@H]1C[C@H](C(=O)N2CCc3ccccc3[C@@H]2c2ccc(F)cc2)OC[C@@H]1O] | 5587 |
| 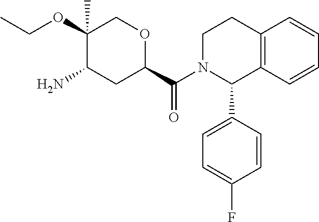 [SMILES: CCO[C@@]1(C)CO[C@@H](C(=O)N2CCc3ccccc3[C@@H]2c2ccc(F)cc2)C[C@@H]1N] | 5588 |
| 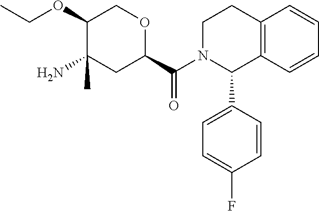 [SMILES: CCO[C@H]1CO[C@@H](C(=O)N2CCc3ccccc3[C@@H]2c2ccc(F)cc2)C[C@]1(C)N] | 5589 |
| 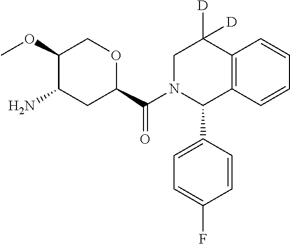 [SMILES: [2H]C1([2H])CN(C(=O)[C@H]2C[C@H](N)[C@@H](OC)CO2)[C@@H](c2ccc(F)cc2)c2ccccc21] | 5592 |
| 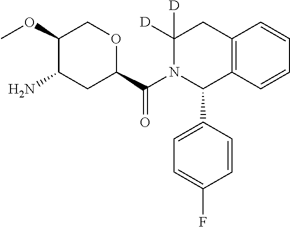 [SMILES: [2H]C1([2H])Cc2ccccc2[C@H](c2ccc(F)cc2)N1C(=O)[C@H]1C[C@H](N)[C@@H](OC)CO1] | 5593 |
| 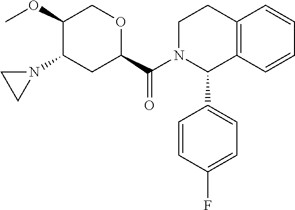 [SMILES: CO[C@H]1CO[C@@H](C(=O)N2CCc3ccccc3[C@@H]2c2ccc(F)cc2)C[C@@H]1N1CC1] | 5594 |
| 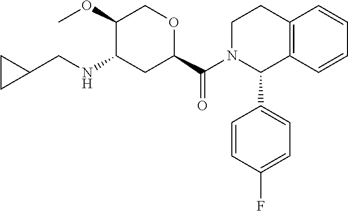 [SMILES: CO[C@H]1CO[C@@H](C(=O)N2CCc3ccccc3[C@@H]2c2ccc(F)cc2)C[C@@H]1NCC1CC1] | 5595 |
| 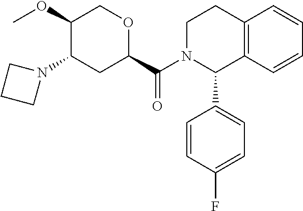 [SMILES: CO[C@H]1CO[C@@H](C(=O)N2CCc3ccccc3[C@@H]2c2ccc(F)cc2)C[C@@H]1N1CCC1] | 5596 |
| 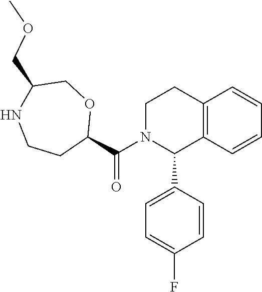 [SMILES: COC[C@H]1CO[C@@H](C(=O)N2CCc3ccccc3[C@@H]2c2ccc(F)cc2)CCN1] | 5597 |
| 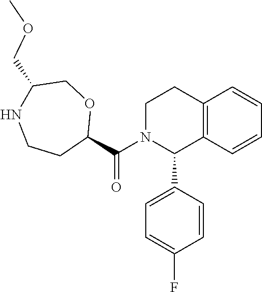 [SMILES: COC[C@@H]1CO[C@@H](C(=O)N2CCc3ccccc3[C@@H]2c2ccc(F)cc2)CCN1] | 5598 |
| 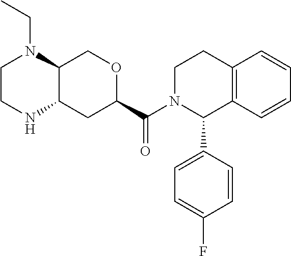 [SMILES: CCN1CCN[C@H]2C[C@H](C(=O)N3CCc4ccccc4[C@@H]3c3ccc(F)cc3)OC[C@@H]21] | 5599 |
| 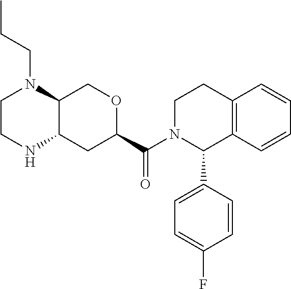 [SMILES: CCCN1CCN[C@H]2C[C@H](C(=O)N3CCc4ccccc4[C@@H]3c3ccc(F)cc3)OC[C@@H]21] | 5600 |
| 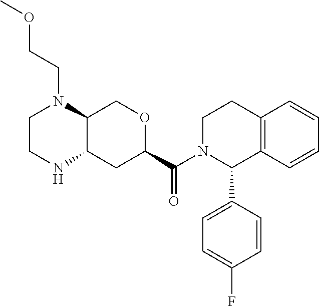 [SMILES: COCCN1CCN[C@H]2C[C@H](C(=O)N3CCc4ccccc4[C@@H]3c3ccc(F)cc3)OC[C@@H]21] | 5601 |
| 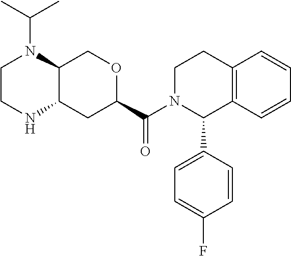 [SMILES: CC(C)N1CCN[C@H]2C[C@H](C(=O)N3CCc4ccccc4[C@@H]3c3ccc(F)cc3)OC[C@@H]21] | 5602 |
| 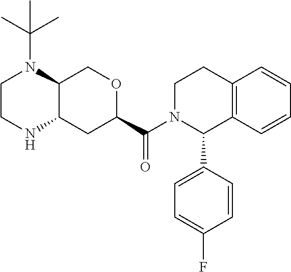 [SMILES: CC(C)(C)N1CCN[C@H]2C[C@H](C(=O)N3CCc4ccccc4[C@@H]3c3ccc(F)cc3)OC[C@@H]21] | 5603 |
| 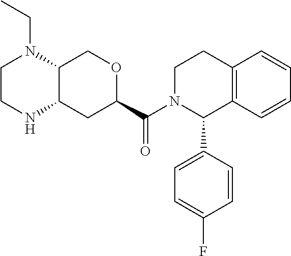 [SMILES: CCN1CCN[C@H]2C[C@H](C(=O)N3CCc4ccccc4[C@@H]3c3ccc(F)cc3)OC[C@H]21] | 5604 |
| 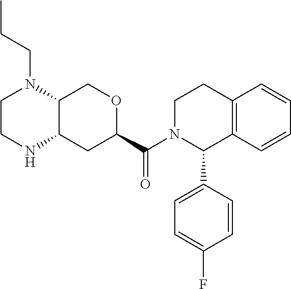 [SMILES: CCCN1CCN[C@H]2C[C@H](C(=O)N3CCc4ccccc4[C@@H]3c3ccc(F)cc3)OC[C@H]21] | 5605 |
| 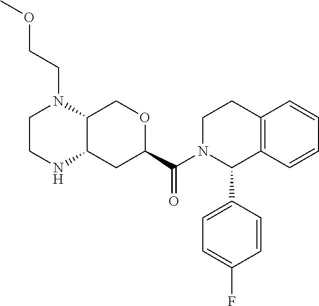 [SMILES: COCCN1CCN[C@H]2C[C@H](C(=O)N3CCc4ccccc4[C@@H]3c3ccc(F)cc3)OC[C@H]21] | 5606 |
| 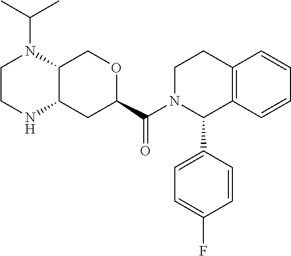 [SMILES: CC(C)N1CCN[C@H]2C[C@H](C(=O)N3CCc4ccccc4[C@@H]3c3ccc(F)cc3)OC[C@H]21] | 5607 |
| 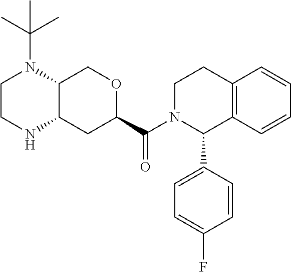 [SMILES: CC(C)(C)N1CCN[C@H]2C[C@H](C(=O)N3CCc4ccccc4[C@@H]3c3ccc(F)cc3)OC[C@H]21] | 5608 |
| 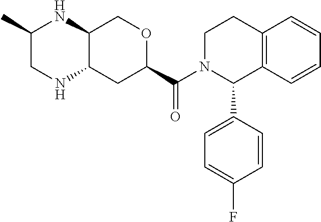 [SMILES: C[C@@H]1CN[C@H]2C[C@H](C(=O)N3CCc4ccccc4[C@@H]3c3ccc(F)cc3)OC[C@@H]2N1] | 5700 |
| 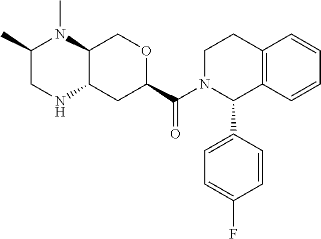 [SMILES: C[C@@H]1CN[C@H]2C[C@H](C(=O)N3CCc4ccccc4[C@@H]3c3ccc(F)cc3)OC[C@@H]2N1C] | 5701 |
| 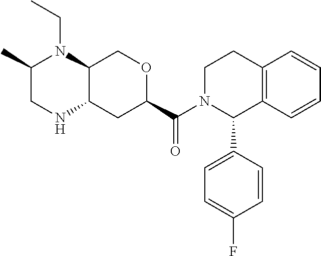 [SMILES: CCN1[C@H](C)CN[C@H]2C[C@H](C(=O)N3CCc4ccccc4[C@@H]3c3ccc(F)cc3)OC[C@@H]21] | 5702 |
| 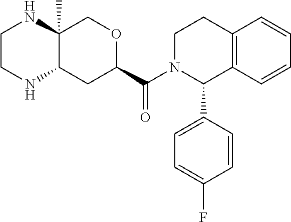 [SMILES: C[C@]12CO[C@@H](C(=O)N3CCc4ccccc4[C@@H]3c3ccc(F)cc3)C[C@@H]1NCCN2] | 5703 |
| 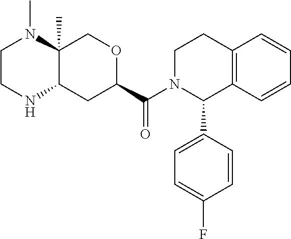 [SMILES: CN1CCN[C@H]2C[C@H](C(=O)N3CCc4ccccc4[C@@H]3c3ccc(F)cc3)OC[C@@]21C] | 5704 |
| 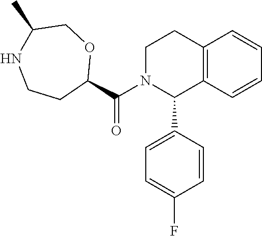 [SMILES: C[C@H]1CO[C@@H](C(=O)N2CCc3ccccc3[C@@H]2c2ccc(F)cc2)CCN1] | 5705 |
| 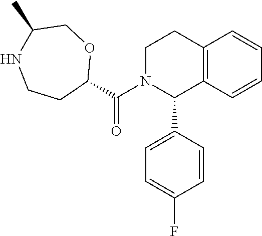 [SMILES: C[C@H]1CO[C@H](C(=O)N2CCc3ccccc3[C@@H]2c2ccc(F)cc2)CCN1] | 5706 |
| 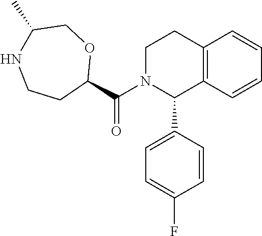 [SMILES: C[C@@H]1CO[C@@H](C(=O)N2CCc3ccccc3[C@@H]2c2ccc(F)cc2)CCN1] | 5707 |
| 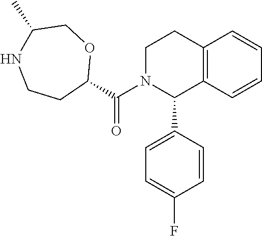 [SMILES: C[C@@H]1CO[C@H](C(=O)N2CCc3ccccc3[C@@H]2c2ccc(F)cc2)CCN1] | 5708 |
| 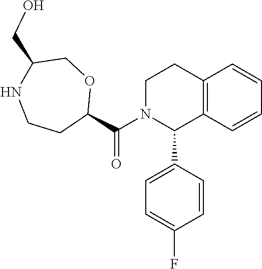 [SMILES: O=C([C@H]1CCN[C@@H](CO)CO1)N1CCc2ccccc2[C@@H]1c1ccc(F)cc1] | 5710 |
| 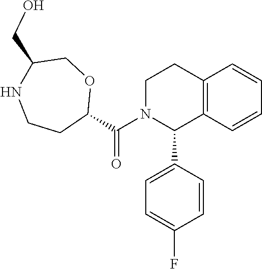 [SMILES: O=C([C@@H]1CCN[C@@H](CO)CO1)N1CCc2ccccc2[C@@H]1c1ccc(F)cc1] | 5711 |
| 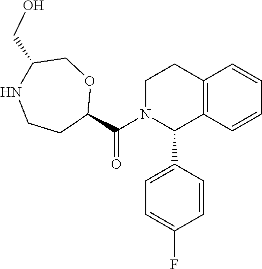 [SMILES: O=C([C@H]1CCN[C@H](CO)CO1)N1CCc2ccccc2[C@@H]1c1ccc(F)cc1] | 5712 |
| 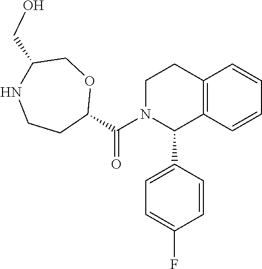 [SMILES: O=C([C@@H]1CCN[C@H](CO)CO1)N1CCc2ccccc2[C@@H]1c1ccc(F)cc1] | 5713 |
| 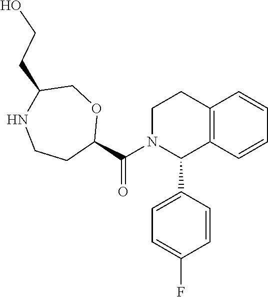 [SMILES: O=C([C@H]1CCN[C@@H](CCO)CO1)N1CCc2ccccc2[C@@H]1c1ccc(F)cc1] | 5714 |
| 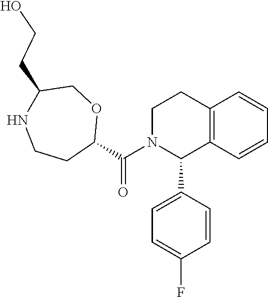 [SMILES: O=C([C@@H]1CCN[C@@H](CCO)CO1)N1CCc2ccccc2[C@@H]1c1ccc(F)cc1] | 5715 |
| 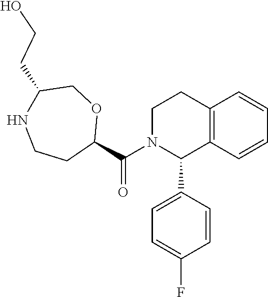 [SMILES: O=C([C@H]1CCN[C@H](CCO)CO1)N1CCc2ccccc2[C@@H]1c1ccc(F)cc1] | 5716 |
| 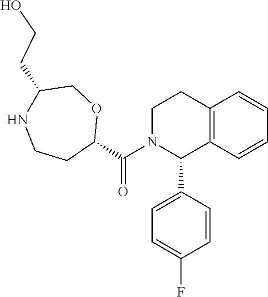 [SMILES: O=C([C@@H]1CCN[C@H](CCO)CO1)N1CCc2ccccc2[C@@H]1c1ccc(F)cc1] | 5717 |
| 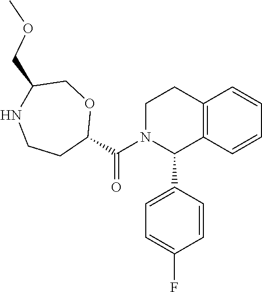 [SMILES: COC[C@H]1CO[C@H](C(=O)N2CCc3ccccc3[C@@H]2c2ccc(F)cc2)CCN1] | 5718 |
| 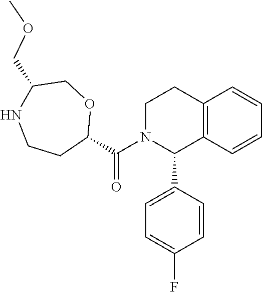 [SMILES: COC[C@@H]1CO[C@H](C(=O)N2CCc3ccccc3[C@@H]2c2ccc(F)cc2)CCN1] | 5719 |
| 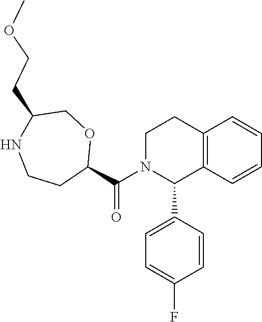 [SMILES: COCC[C@H]1CO[C@@H](C(=O)N2CCc3ccccc3[C@@H]2c2ccc(F)cc2)CCN1] | 5720 |
| 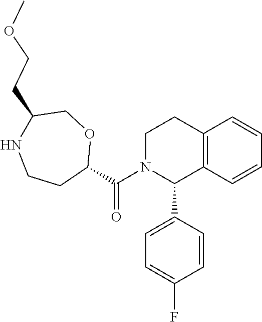 [SMILES: COCC[C@H]1CO[C@H](C(=O)N2CCc3ccccc3[C@@H]2c2ccc(F)cc2)CCN1] | 5721 |
| 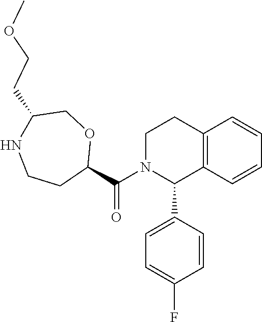 [SMILES: COCC[C@@H]1CO[C@@H](C(=O)N2CCc3ccccc3[C@@H]2c2ccc(F)cc2)CCN1] | 5722 |
| 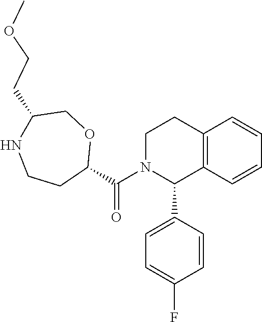 [SMILES: COCC[C@@H]1CO[C@H](C(=O)N2CCc3ccccc3[C@@H]2c2ccc(F)cc2)CCN1] | 5723 |
| 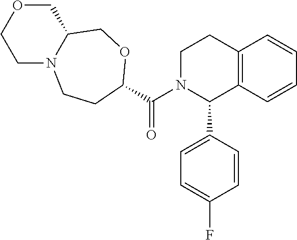 [SMILES: O=C([C@@H]1CCN2CCOC[C@@H]2CO1)N1CCc2ccccc2[C@@H]1c1ccc(F)cc1] | 5724 |
| 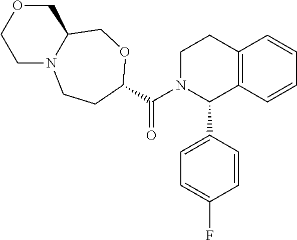 [SMILES: O=C([C@@H]1CCN2CCOC[C@H]2CO1)N1CCc2ccccc2[C@@H]1c1ccc(F)cc1] | 5725 |
| 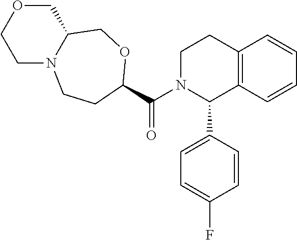 [SMILES: O=C([C@H]1CCN2CCOC[C@@H]2CO1)N1CCc2ccccc2[C@@H]1c1ccc(F)cc1] | 5726 |
| 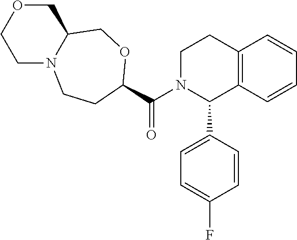 [SMILES: O=C([C@H]1CCN2CCOC[C@H]2CO1)N1CCc2ccccc2[C@@H]1c1ccc(F)cc1] | 5727 |
| 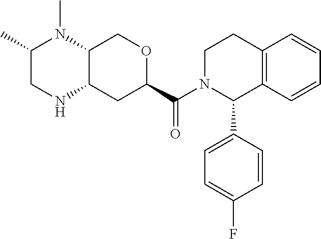 [SMILES: C[C@H]1CN[C@H]2C[C@H](C(=O)N3CCc4ccccc4[C@@H]3c3ccc(F)cc3)OC[C@H]2N1C] | 5728 |
| 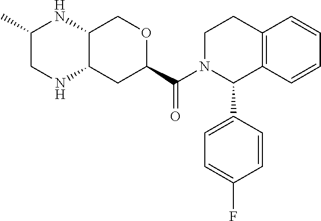 [SMILES: C[C@H]1CN[C@H]2C[C@H](C(=O)N3CCc4ccccc4[C@@H]3c3ccc(F)cc3)OC[C@H]2N1] | 5729 |
| 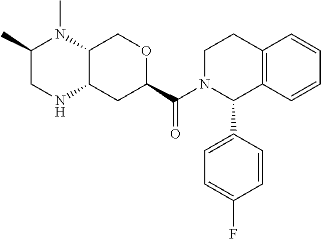 [SMILES: C[C@@H]1CN[C@H]2C[C@H](C(=O)N3CCc4ccccc4[C@@H]3c3ccc(F)cc3)OC[C@H]2N1C] | 5730 |
| 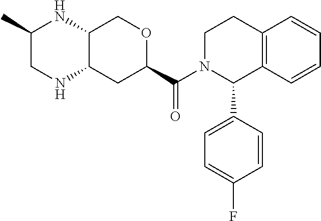 [SMILES: C[C@@H]1CN[C@H]2C[C@H](C(=O)N3CCc4ccccc4[C@@H]3c3ccc(F)cc3)OC[C@H]2N1] | 5731 |
| 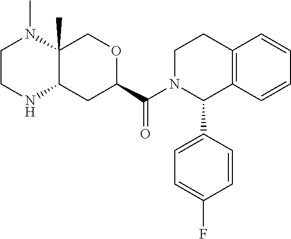 [SMILES: CN1CCN[C@H]2C[C@H](C(=O)N3CCc4ccccc4[C@@H]3c3ccc(F)cc3)OC[C@]21C] | 5732 |
| 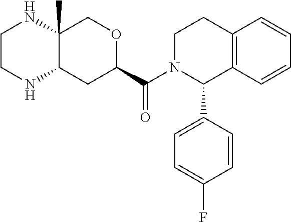 [SMILES: C[C@@]12CO[C@@H](C(=O)N3CCc4ccccc4[C@@H]3c3ccc(F)cc3)C[C@@H]1NCCN2] | 5733 |
| 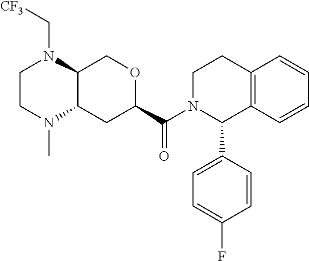 [SMILES: CN1CCN(CC(F)(F)F)[C@H]2CO[C@@H](C(=O)N3CCc4ccccc4[C@@H]3c3ccc(F)cc3)C[C@@H]21] | 5735 |
| 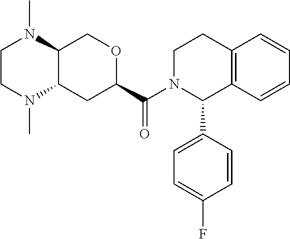 [SMILES: CN1CCN(C)[C@H]2C[C@H](C(=O)N3CCc4ccccc4[C@@H]3c3ccc(F)cc3)OC[C@@H]21] | 5737 |
| 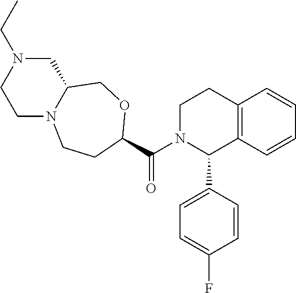 [SMILES: CCN1CCN2CC[C@H](C(=O)N3CCc4ccccc4[C@@H]3c3ccc(F)cc3)OC[C@H]2C1] | 5738 |
| 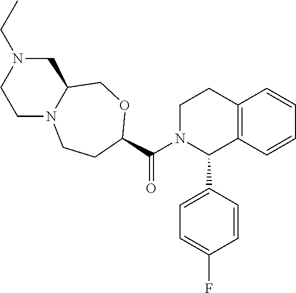 [SMILES: CCN1CCN2CC[C@H](C(=O)N3CCc4ccccc4[C@@H]3c3ccc(F)cc3)OC[C@@H]2C1] | 5739 |
| 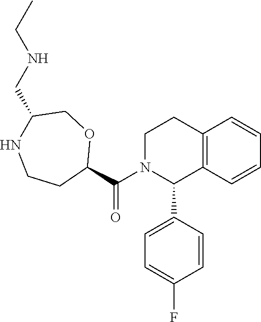 [SMILES: CCNC[C@@H]1CO[C@@H](C(=O)N2CCc3ccccc3[C@@H]2c2ccc(F)cc2)CCN1] | 5740 |
| 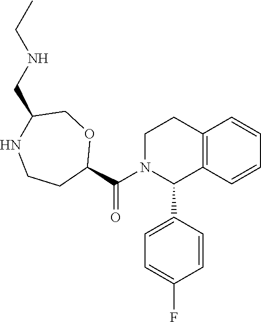 [SMILES: CCNC[C@H]1CO[C@@H](C(=O)N2CCc3ccccc3[C@@H]2c2ccc(F)cc2)CCN1] | 5741 |
| 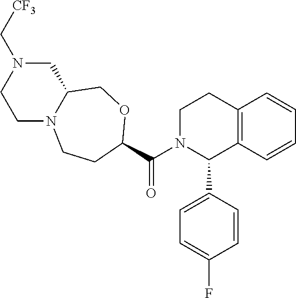 [SMILES: O=C([C@H]1CCN2CCN(CC(F)(F)F)C[C@@H]2CO1)N1CCc2ccccc2[C@@H]1c1ccc(F)cc1] | 5742 |
| 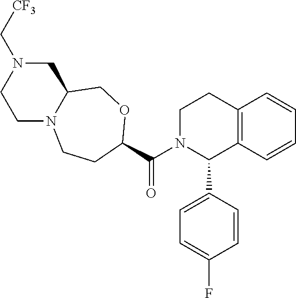 [SMILES: O=C([C@H]1CCN2CCN(CC(F)(F)F)C[C@H]2CO1)N1CCc2ccccc2[C@@H]1c1ccc(F)cc1] | 5743 |
| 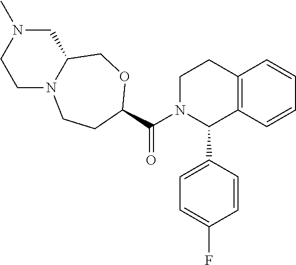 [SMILES: CN1CCN2CC[C@H](C(=O)N3CCc4ccccc4[C@@H]3c3ccc(F)cc3)OC[C@H]2C1] | 5744 |
| 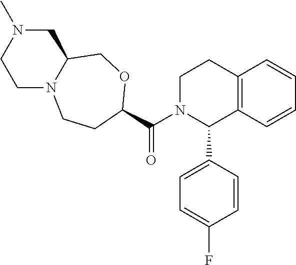 [SMILES: CN1CCN2CC[C@H](C(=O)N3CCc4ccccc4[C@@H]3c3ccc(F)cc3)OC[C@@H]2C1] | 5745 |
| 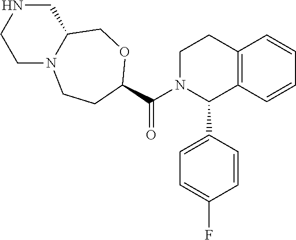 [SMILES: O=C([C@H]1CCN2CCNC[C@@H]2CO1)N1CCc2ccccc2[C@@H]1c1ccc(F)cc1] | 5746 |
| 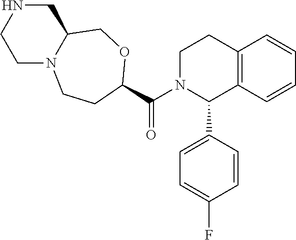 [SMILES: O=C([C@H]1CCN2CCNC[C@H]2CO1)N1CCc2ccccc2[C@@H]1c1ccc(F)cc1] | 5747 |
| 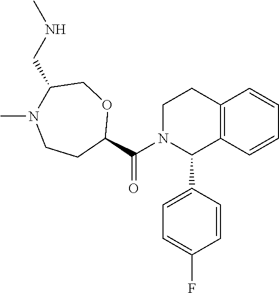 [SMILES: CNC[C@@H]1CO[C@@H](C(=O)N2CCc3ccccc3[C@@H]2c2ccc(F)cc2)CCN1C] | 5748 |
| 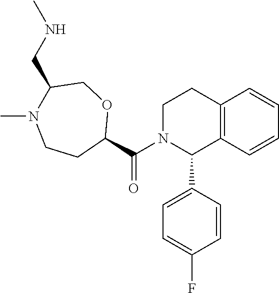 [SMILES: CNC[C@H]1CO[C@@H](C(=O)N2CCc3ccccc3[C@@H]2c2ccc(F)cc2)CCN1C] | 5749 |
| 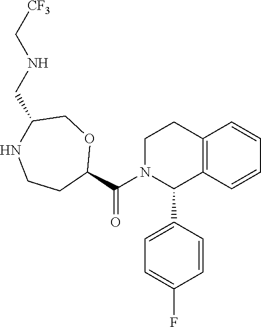 [SMILES: O=C([C@H]1CCN[C@H](CNCC(F)(F)F)CO1)N1CCc2ccccc2[C@@H]1c1ccc(F)cc1] | 5750 |
| 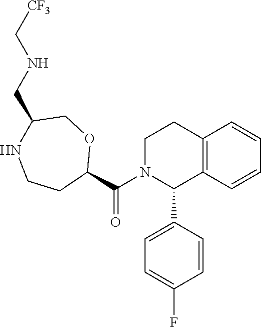 [SMILES: O=C([C@H]1CCN[C@@H](CNCC(F)(F)F)CO1)N1CCc2ccccc2[C@@H]1c1ccc(F)cc1] | 5751 |
| 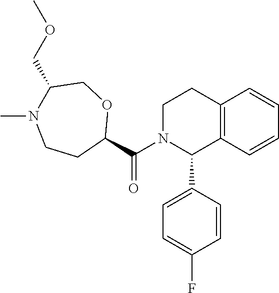 [SMILES: COC[C@@H]1CO[C@@H](C(=O)N2CCc3ccccc3[C@@H]2c2ccc(F)cc2)CCN1C] | 5752 |
| 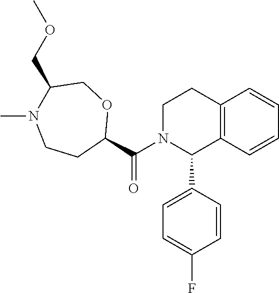 [SMILES: COC[C@H]1CO[C@@H](C(=O)N2CCc3ccccc3[C@@H]2c2ccc(F)cc2)CCN1C] | 5753 |
| 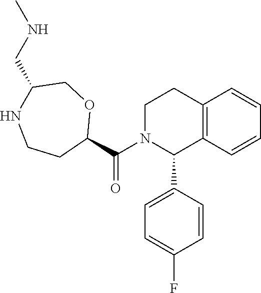 [SMILES: CNC[C@@H]1CO[C@@H](C(=O)N2CCc3ccccc3[C@@H]2c2ccc(F)cc2)CCN1] | 5754 |
| 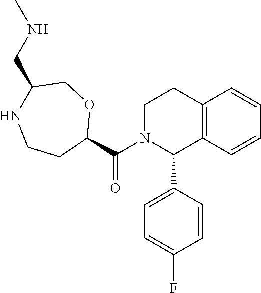 [SMILES: CNC[C@H]1CO[C@@H](C(=O)N2CCc3ccccc3[C@@H]2c2ccc(F)cc2)CCN1] | 5755 |
| 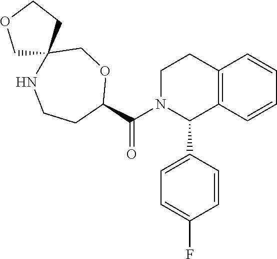 [SMILES: O=C([C@H]1CCN[C@@]2(CCOC2)CO1)N1CCc2ccccc2[C@@H]1c1ccc(F)cc1] | 5756 |
| 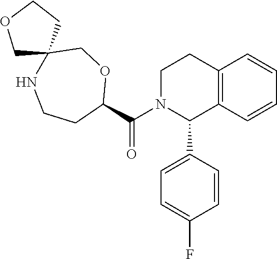 [SMILES: O=C([C@H]1CCN[C@]2(CCOC2)CO1)N1CCc2ccccc2[C@@H]1c1ccc(F)cc1] | 5757 |
| 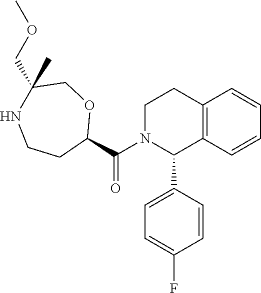 [SMILES: COC[C@]1(C)CO[C@@H](C(=O)N2CCc3ccccc3[C@@H]2c2ccc(F)cc2)CCN1] | 5758 |
| 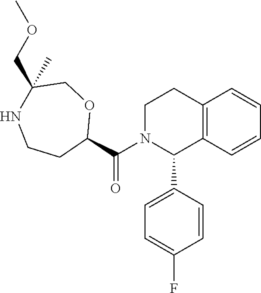 [SMILES: COC[C@@]1(C)CO[C@@H](C(=O)N2CCc3ccccc3[C@@H]2c2ccc(F)cc2)CCN1] | 5759 |
| 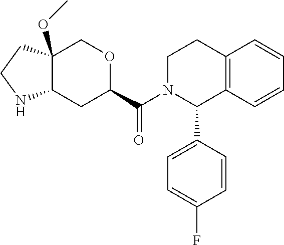 [SMILES: CO[C@]12CCN[C@H]1C[C@H](C(=O)N1CCc3ccccc3[C@@H]1c1ccc(F)cc1)OC2] | 5760 |
| 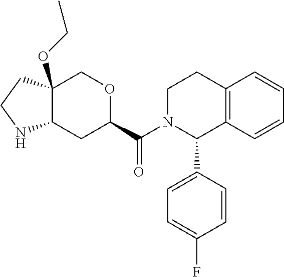 [SMILES: CCO[C@]12CCN[C@H]1C[C@H](C(=O)N1CCc3ccccc3[C@@H]1c1ccc(F)cc1)OC2] | 5761 |
| 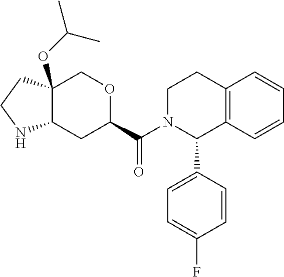 [SMILES: CC(C)O[C@]12CCN[C@H]1C[C@H](C(=O)N1CCc3ccccc3[C@@H]1c1ccc(F)cc1)OC2] | 5762 |
| 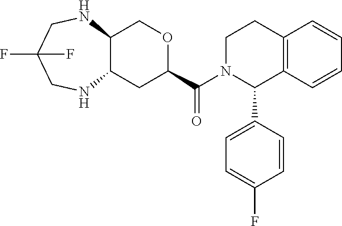 | 5763 |
| 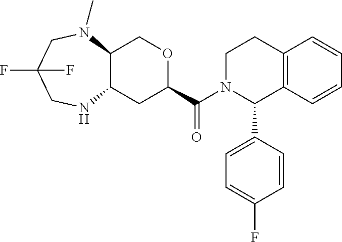 | 5764 |
| 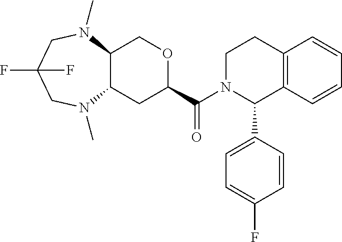 | 5765 |
| 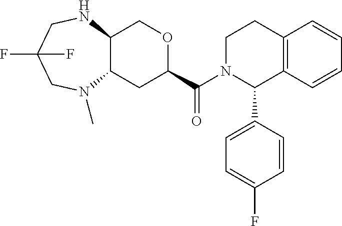 | 5766 |
| 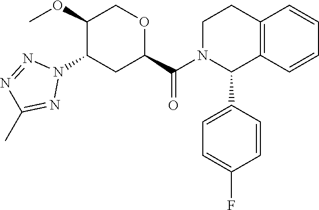 [SMILES: CO[C@H]1CO[C@@H](C(=O)N2CCc3ccccc3[C@@H]2c2ccc(F)cc2)C[C@@H]1n1nnc(C)n1] | 5767 |
| 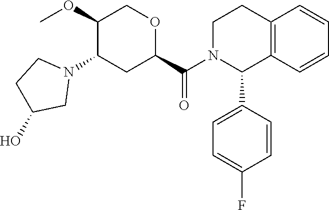 [SMILES: CO[C@H]1CO[C@@H](C(=O)N2CCc3ccccc3[C@@H]2c2ccc(F)cc2)C[C@@H]1N1CC[C@@H](O)C1] | 5768 |
| 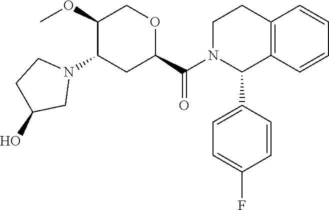 [SMILES: CO[C@H]1CO[C@@H](C(=O)N2CCc3ccccc3[C@@H]2c2ccc(F)cc2)C[C@@H]1N1CC[C@H](O)C1] | 5769 |
| 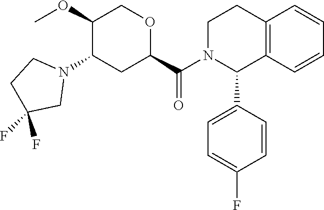 | 5770 |
| 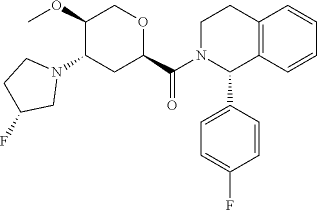 [SMILES: CO[C@H]1CO[C@@H](C(=O)N2CCc3ccccc3[C@@H]2c2ccc(F)cc2)C[C@@H]1N1CC[C@@H](F)C1] | 5771 |
| 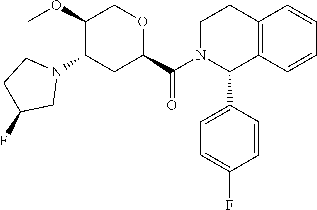 [SMILES: CO[C@H]1CO[C@@H](C(=O)N2CCc3ccccc3[C@@H]2c2ccc(F)cc2)C[C@@H]1N1CC[C@H](F)C1] | 5772 |
| 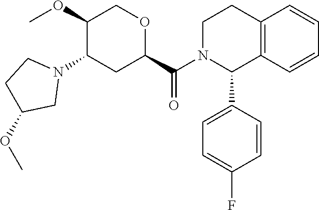 [SMILES: CO[C@@H]1CCN([C@H]2C[C@H](C(=O)N3CCc4ccccc4[C@@H]3c3ccc(F)cc3)OC[C@@H]2OC)C1] | 5773 |
| 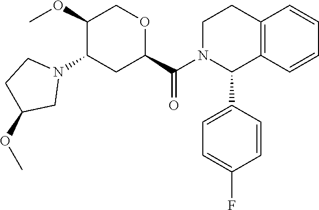 [SMILES: CO[C@H]1CCN([C@H]2C[C@H](C(=O)N3CCc4ccccc4[C@@H]3c3ccc(F)cc3)OC[C@@H]2OC)C1] | 5774 |
| 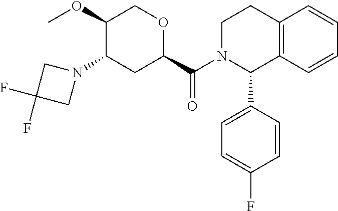 [SMILES: CO[C@H]1CO[C@@H](C(=O)N2CCc3ccccc3[C@@H]2c2ccc(F)cc2)C[C@@H]1N1CC(F)(F)C1] | 5775 |
| 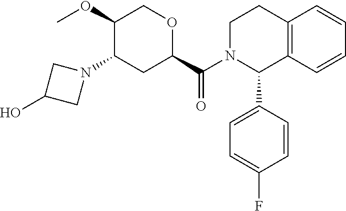 [SMILES: CO[C@H]1CO[C@@H](C(=O)N2CCc3ccccc3[C@@H]2c2ccc(F)cc2)C[C@@H]1N1CC(O)C1] | 5776 |
| 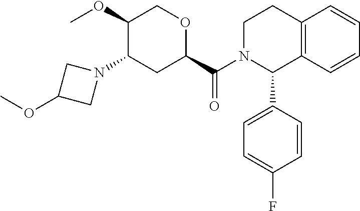 [SMILES: COC1CN([C@H]2C[C@H](C(=O)N3CCc4ccccc4[C@@H]3c3ccc(F)cc3)OC[C@@H]2OC)C1] | 5777 |
| 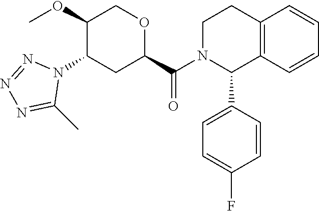 [SMILES: CO[C@H]1CO[C@@H](C(=O)N2CCc3ccccc3[C@@H]2c2ccc(F)cc2)C[C@@H]1n1nnnc1C] | 5778 |
| 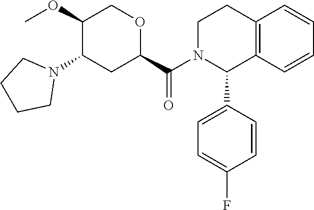 [SMILES: CO[C@H]1CO[C@@H](C(=O)N2CCc3ccccc3[C@@H]2c2ccc(F)cc2)C[C@@H]1N1CCCC1] | 5779 |
| 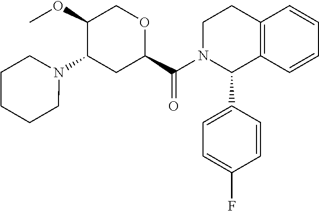 [SMILES: CO[C@H]1CO[C@@H](C(=O)N2CCc3ccccc3[C@@H]2c2ccc(F)cc2)C[C@@H]1N1CCCCC1] | 5780 |
| 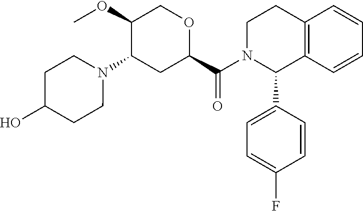 [SMILES: CO[C@H]1CO[C@@H](C(=O)N2CCc3ccccc3[C@@H]2c2ccc(F)cc2)C[C@@H]1N1CCC(O)CC1] | 5781 |
| 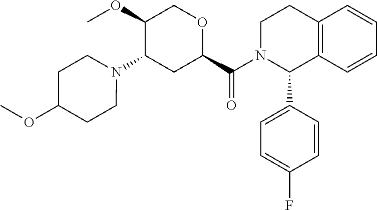 [SMILES: COC1CCN([C@H]2C[C@H](C(=O)N3CCc4ccccc4[C@@H]3c3ccc(F)cc3)OC[C@@H]2OC)CC1] | 5782 |
| 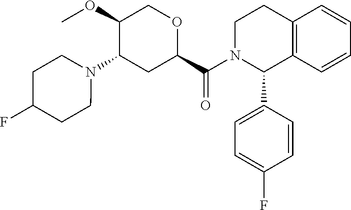 [SMILES: CO[C@H]1CO[C@@H](C(=O)N2CCc3ccccc3[C@@H]2c2ccc(F)cc2)C[C@@H]1N1CCC(F)CC1] | 5783 |
| 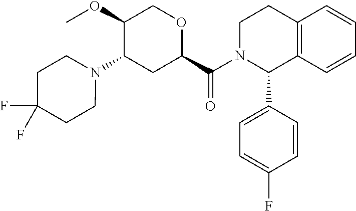 [SMILES: CO[C@H]1CO[C@@H](C(=O)N2CCc3ccccc3[C@@H]2c2ccc(F)cc2)C[C@@H]1N1CCC(F)(F)CC1] | 5784 |
| 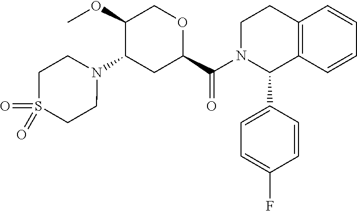 [SMILES: CO[C@H]1CO[C@@H](C(=O)N2CCc3ccccc3[C@@H]2c2ccc(F)cc2)C[C@@H]1N1CCS(=O)(=O)CC1] | 5785 |
| 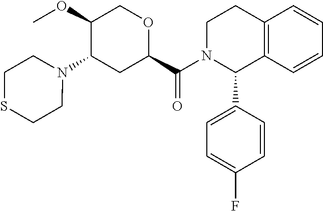 [SMILES: CO[C@H]1CO[C@@H](C(=O)N2CCc3ccccc3[C@@H]2c2ccc(F)cc2)C[C@@H]1N1CCSCC1] | 5786 |
| 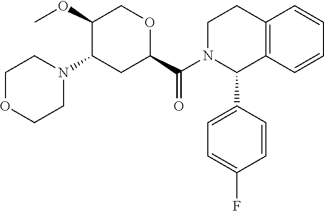 [SMILES: CO[C@H]1CO[C@@H](C(=O)N2CCc3ccccc3[C@@H]2c2ccc(F)cc2)C[C@@H]1N1CCOCC1] | 5787 |
| 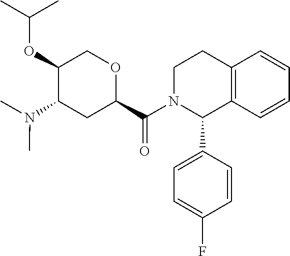 [SMILES: CC(C)O[C@H]1CO[C@@H](C(=O)N2CCc3ccccc3[C@@H]2c2ccc(F)cc2)C[C@@H]1N(C)C] | 5788 |
| 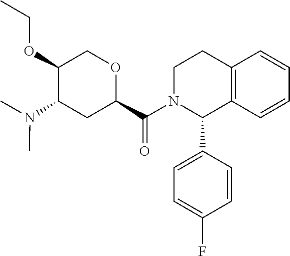 [SMILES: CCO[C@H]1CO[C@@H](C(=O)N2CCc3ccccc3[C@@H]2c2ccc(F)cc2)C[C@@H]1N(C)C] | 5789 |
| 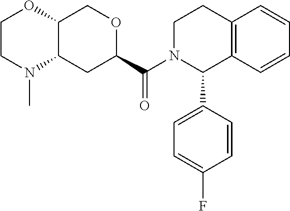 [SMILES: CN1CCO[C@@H]2CO[C@@H](C(=O)N3CCc4ccccc4[C@@H]3c3ccc(F)cc3)C[C@@H]21] | 5790 |
| 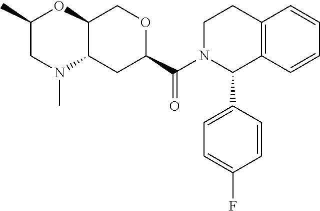 [SMILES: C[C@@H]1CN(C)[C@H]2C[C@H](C(=O)N3CCc4ccccc4[C@@H]3c3ccc(F)cc3)OC[C@@H]2O1] | 5791 |
| 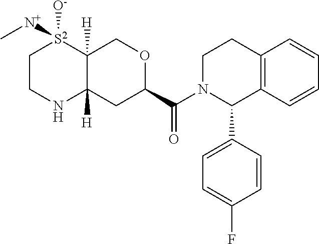 | 5792 |
| 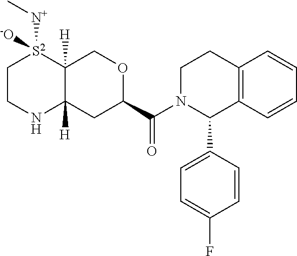 | 5793 |
| 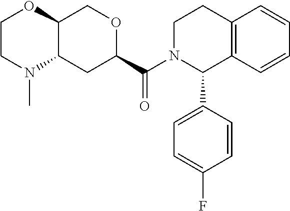 [SMILES: CN1CCO[C@H]2CO[C@@H](C(=O)N3CCc4ccccc4[C@@H]3c3ccc(F)cc3)C[C@@H]21] | 5800 |
| 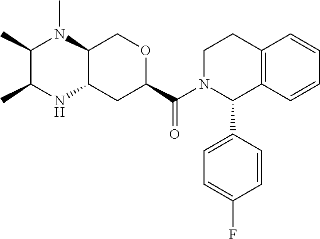 [SMILES: C[C@@H]1N[C@H]2C[C@H](C(=O)N3CCc4ccccc4[C@@H]3c3ccc(F)cc3)OC[C@@H]2N(C)[C@@H]1C] | 5801 |
| 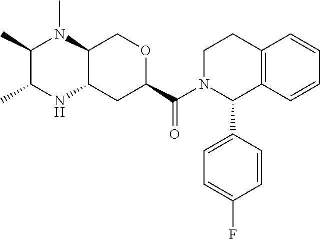 [SMILES: C[C@@H]1[C@@H](C)N[C@H]2C[C@H](C(=O)N3CCc4ccccc4[C@@H]3c3ccc(F)cc3)OC[C@@H]2N1C] | 5802 |
| 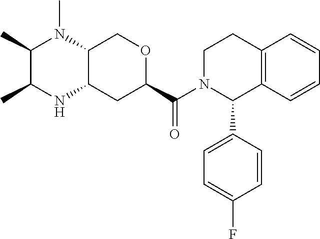 [SMILES: C[C@@H]1N[C@H]2C[C@H](C(=O)N3CCc4ccccc4[C@@H]3c3ccc(F)cc3)OC[C@H]2N(C)[C@@H]1C] | 5803 |
| 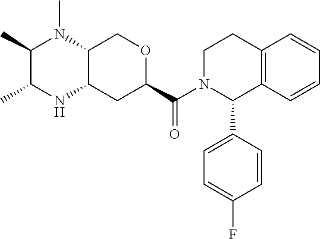 [SMILES: C[C@@H]1[C@@H](C)N[C@H]2C[C@H](C(=O)N3CCc4ccccc4[C@@H]3c3ccc(F)cc3)OC[C@H]2N1C] | 5804 |
| 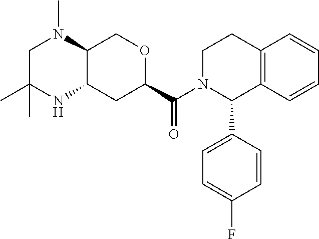 [SMILES: CN1CC(C)(C)N[C@H]2C[C@H](C(=O)N3CCc4ccccc4[C@@H]3c3ccc(F)cc3)OC[C@@H]21] | 5805 |
| 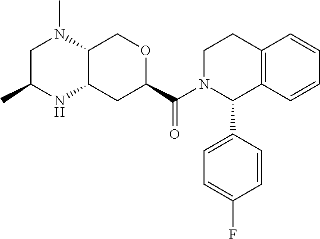 [SMILES: C[C@H]1CN(C)[C@@H]2CO[C@@H](C(=O)N3CCc4ccccc4[C@@H]3c3ccc(F)cc3)C[C@@H]2N1] | 5806 |
| 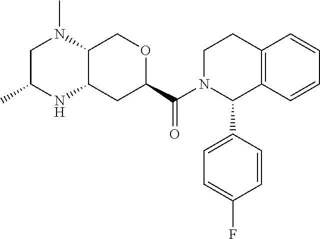 [SMILES: C[C@@H]1CN(C)[C@@H]2CO[C@@H](C(=O)N3CCc4ccccc4[C@@H]3c3ccc(F)cc3)C[C@@H]2N1] | 5807 |
| 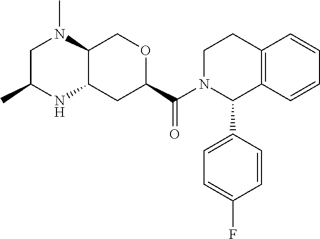 [SMILES: C[C@H]1CN(C)[C@H]2CO[C@@H](C(=O)N3CCc4ccccc4[C@@H]3c3ccc(F)cc3)C[C@@H]2N1] | 5808 |
| 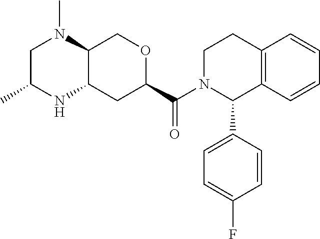 [SMILES: C[C@@H]1CN(C)[C@H]2CO[C@@H](C(=O)N3CCc4ccccc4[C@@H]3c3ccc(F)cc3)C[C@@H]2N1] | 5809 |
| 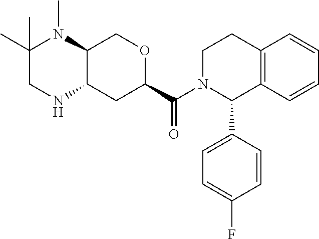 [SMILES: CN1[C@H]2CO[C@@H](C(=O)N3CCc4ccccc4[C@@H]3c3ccc(F)cc3)C[C@@H]2NCC1(C)C] | 5810 |
| 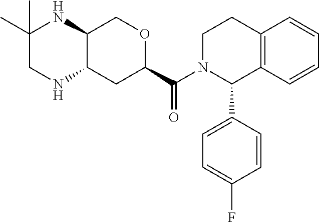 [SMILES: CC1(C)CN[C@H]2C[C@H](C(=O)N3CCc4ccccc4[C@@H]3c3ccc(F)cc3)OC[C@@H]2N1] | 5812 |
| 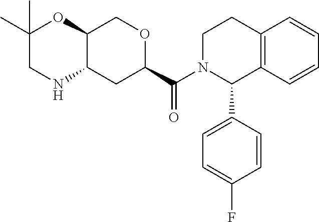 [SMILES: CC1(C)CN[C@H]2C[C@H](C(=O)N3CCc4ccccc4[C@@H]3c3ccc(F)cc3)OC[C@@H]2O1] | 5814 |
| 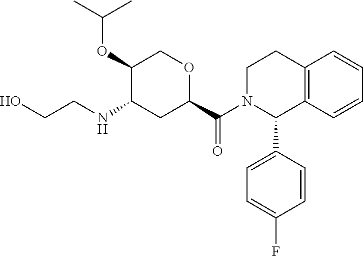 [SMILES: CC(C)O[C@H]1CO[C@@H](C(=O)N2CCc3ccccc3[C@@H]2c2ccc(F)cc2)C[C@@H]1NCCO] | 5815 |
| 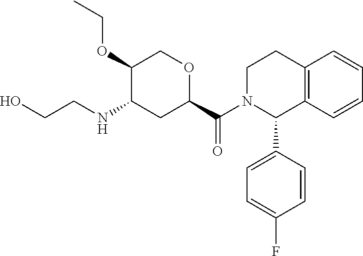 [SMILES: CCO[C@H]1CO[C@@H](C(=O)N2CCc3ccccc3[C@@H]2c2ccc(F)cc2)C[C@@H]1NCCO] | 5816 |
| 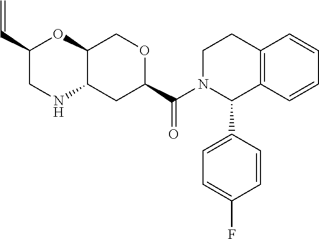 [SMILES: C=C[C@@H]1CN[C@H]2C[C@H](C(=O)N3CCc4ccccc4[C@@H]3c3ccc(F)cc3)OC[C@@H]2O1] | 5817 |
| 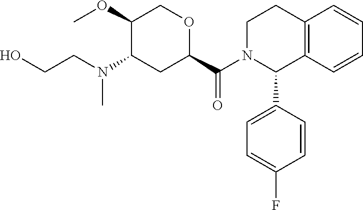 [SMILES: CO[C@H]1CO[C@@H](C(=O)N2CCc3ccccc3[C@@H]2c2ccc(F)cc2)C[C@@H]1N(C)CCO] | 5818 |
| 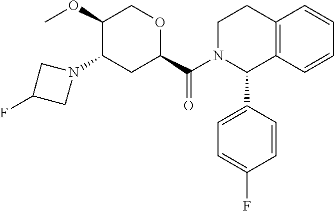 [SMILES: CO[C@H]1CO[C@@H](C(=O)N2CCc3ccccc3[C@@H]2c2ccc(F)cc2)C[C@@H]1N1CC(F)C1] | 5819 |

In some cases, the compound is selected from compound 5605, 5602, 5599, 5575, 5564, 5550, 5472, 5545, 5543, 5461, 5267, 5448, 5475, 5087, 5077, 5051, 5045, 5042, 5021, and 5012, and pharmaceutically acceptable salts thereof. In some cases, the compound is selected from compound 5599, 5564, 5472, 5077, and 5087, and pharmaceutically acceptable salts thereof. In some cases, the compound is compound 5599 or a pharmaceutically acceptable salt thereof. In some cases, the compound is compound 5564 or a pharmaceutically acceptable salt thereof. In some cases, the compound is compound 5472 or a pharmaceutically acceptable salt thereof. In some cases, the compound is compound 5077 or a pharmaceutically acceptable salt thereof. In some cases, the compound is compound 5087 or a pharmaceutically acceptable salt thereof.

As used herein, the term “alkyl” refers to straight chained and branched saturated hydrocarbon groups containing one to six carbon atoms. The term Cₙ means the alkyl group has “n” carbon atoms. For example, C₆ alkyl refers to an alkyl group that has 6 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), and 3-methylpentyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. A C₀ alkylene indicates a direct bond. Unless otherwise indicated, an alkylene group can be an unsubstituted alkylene group or a substituted alkylene group. Particular substitutions on the alkylene group can be specified, e.g., alkylene-halo, alkylene-CN, or the like.

The term “alkene” or “alkenyl” used herein refers to an unsaturated aliphatic group analogous in length and possible substitution to an alkyl group described above, but that contains at least one double bond. For example, the term “alkenyl” includes straight chain alkenyl groups (e.g., ethenyl, propenyl, butenyl, pentenyl, hexenyl), and branched alkenyl groups. For example, a straight chain or branched alkenyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). Unless otherwise indicated, an alkenyl group can be an unsubstituted alkenyl group or a substituted alkenyl group.

As used herein, the term “haloalkyl” refers to an alkyl group substituted with one or more halogen substituents. Haloalkyl is alternatively referred to as “alkylene-halo.” For example, C₁-C₆haloalkyl refers to a C₁-C₆ alkyl group substituted with one or more halogen atoms, e.g., 1, 2, 3, 4, 5, or 6 halogen atoms. Non-limiting examples of haloalkyl groups include fluoromethyl, difluoromethyl, trifluoromethyl, chloromethyl, dichloromethyl, and trichloromethyl groups. Similarly, haloalkoxy refers to an alkoxy group substituted with one or more halogen atoms e.g., 1, 2, 3, 4, 5, or 6 halogen atoms.

As used herein, the term “halo” or “halogen” refers to fluorine, chlorine, bromine, or iodine.

As used herein, the term “oxo” refers to ═O substituent, e.g., a carbon can be substituted with an oxo to form a carbonyl (C═O) group.

As used herein, the term “carbocycle” or “carbocyclyl” refers to a cyclic hydrocarbon group containing three to eleven carbon atoms (e.g., 3, 4, 5, 6, 7, 8, 9, 10, or 11 carbon atoms). The term “n-membered carbocycle” means the carbocycle group has “n” carbon atoms. For example, 5-membered carbocycle refers to a carbocycle group that has 5 carbon atoms in the ring. 6- to 8-membered carbocycle refers to carbocycle groups having a number of carbon atoms encompassing the entire range (e.g., 6 to 8 carbon atoms), as well as all subgroups (e.g., 6-7, 6-8, 7-8, 6, 7, and 8 carbon atoms). Nonlimiting examples of carbocycle groups include cyclopropyl, cyclobutyl, cyclopentyl, cyclohexyl, cycloheptyl, and cyclooctyl. Unless otherwise indicated, a carbocycle group can be an unsubstituted carbocycle group or a substituted carbocycle group. The carbocycle groups described herein can be isolated or fused to another carbocycle group. In particular, the carbocycles described herein can have a fused, bridged, or spiro structure.

As used herein, the term “heterocycle” is defined similarly as carbocycle, except the ring contains one to four heteroatoms independently selected from oxygen, nitrogen, and sulfur. In particular, the term “heterocycle” refers to a ring containing a total of four to twelve atoms (e.g., four to six, or six to eight), of which 1, 2, 3 or 4 of those atoms are heteroatoms independently selected from the group consisting of oxygen, nitrogen, and sulfur, and the remaining atoms in the ring are carbon atoms. Heterocycle rings as disclosed herein can be in a monocyclic, fused (e.g., bicyclic), bridged, or spiro form, yet still exhibit the 4-12 members of the ring(s) and heteroatoms as discussed herein. Nonlimiting examples of heterocycle groups include azetidine, piperdine, piperazine, pyrazolidine, tetrahydrofuran, tetrahydropyran, dihydrofuran, morpholine, quinuclidine, and the like. Heterocycle groups can be saturated or partially unsaturated ring systems optionally substituted with, for example, one to three groups, such as halo, C₁₋₆alkyl, C₁₋₆haloalkyl, OH, C₁₋₆alkylene-OH, C₁₋₆alkoxy, C₁₋₆ haloalkoxy, C₁₋₆alkoxy-C₁₋₆alkyl, —NR^{a}R^{b} (e.g., —NH₂ or —NHMe), C₁₋₆alkylene-NR^{a}R^{b} (e.g., C₁₋₆alkylene-NH₂ or C₁₋₆ alkylene-NHMe), S—C₁₋₆alkyl, C₂₋₆alkene, C(O)—C₁₋₆haloalkyl, or SO₂—C₁₋₆alkyl. Other contemplated substituents on a heterocycle ring are discussed elsewhere in this disclosure.

As used herein, the term “substituted,” when used to modify a chemical functional group, refers to the replacement of at least one hydrogen radical on the functional group with a substituent. Unless otherwise specified for a particular moiety, substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

Compounds of the present disclosure can exist in particular geometric or stereoisomeric forms having one or more asymmetric carbon atoms. The present disclosure contemplates such forms, including cis- and trans-isomers, R- and S-enantiomers, diastereomers, racemic mixtures thereof, and other mixtures thereof, as falling within the scope of the disclosed compounds. Additional asymmetric carbon atoms may be present in substituents such as alkyl groups. All such isomers, as well as mixtures thereof, are intended for inclusion herein.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Formulations, Dosing, and Routes of Administration

Further provided are pharmaceutical formulations (alternatively referred to as compositions throughout herein) comprising a compound as described herein or pharmaceutically acceptable salt thereof, and a pharmaceutically acceptable excipient.

The compounds described herein can be administered to a subject in a therapeutically effective amount, alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to affect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, for example, from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds disclosed herein (e.g., compounds of Formula I and as shown in Table A) can increase the amount of progranulin or granulin in a subject. In some cases, the compounds increase the amount of progranulin in a subject. In some cases, the compounds increase the amount of granulin in a subject. In some cases, the compounds affect cells to increase secretion of progranulin. As such, the compounds disclosed herein, ((e.g., compounds of Formula I and as shown in Table A) can be useful in treating disorders associated with aberrant (e.g., reduced) progranulin secretion or activity.

Specifically contemplated are methods of using a therapeutically effective amount of a compound disclosed herein to modulate progranulin (e.g., to increase secretion of progranulin), for use as a therapeutic in a subject. As used herein, the term “therapeutically effective amount” means an amount of a compound or combination of therapeutically active compounds (e.g., a progranulin modulator or combination of modulators) that ameliorates, attenuates or eliminates one or more symptoms of a particular disease or condition (e.g., progranulin- or granulin-associated disorders), or prevents or delays the onset of one of more symptoms of a particular disease or condition.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., increasing secretion of progranulin. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

Contemplated disorders associated with aberrant progranulin activity include Alzheimer's disease (AD), Parkinson's disease (PD) and PD-related disorders, Amytrophic lateral sclerosis (ALS), Frontotemperal lobe dementia (FTLD), Lewy body dementia (LBD), Prion disease, Motor neurone diseases (MND), Huntington's disease (HD), Spinocerebellar ataxia (SCA), Spinal muscular atrophy (SMA) and other neurodegenerative diseases. Other disorders contemplated include lysosomal dys-or misfunction disorders, such lysosomal storage diseases (e.g., Paget's disease, Gaucher's disease, Nieman's Pick disease, Tay-Sachs Disease, Fabry Disease, Pompes disease, and Naso-Hakula disease). Other diseases contemplated include those associated with inclusions and/or misfunction of C9orf72, TDP-43, FUS, UBQLN2, VCP, CHMP28, and/or MAPT. Other diseases include acute neurological disorders such as stroke, cerebral hemorrhage, traumatic brain injury and other head traumas as well as diseases of the brain such as glioblastoma and neuroblastomas.

In some cases, the progranulin-associated disorder is Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), a lysosomal storage disease, nephropathy, a disease associated with inclusions and/or misfunction of C9orf72, TDP-43, FUS, UBQLN2, VCP, CHMP28, and/or MAPT, an acute neurological disorder, glioblastoma, or neuroblastoma. In some cases, the Parkinson's disease is Parkinson's disease with GBA mutation. In some cases, the lysosomal storage disease is Paget's disease, Gaucher's disease, Nieman's Pick disease, Tay-Sachs Disease, Fabry Disease, Pompes disease, or Naso-Hakula disease. In some cases, the acute neurological disorder is stroke, cerebral hemorrhage, traumatic brain injury or head trauma. In some cases, the progranulin-associated disorder is Frontotemporal dementia (FTD). In some cases, the progranulin-associated disorder is Frontotemporal dementia-Granulin subtype (FTD-GRN).

The compounds disclosed herein (e.g., compounds of Formula I and as shown in Table A) can increase the levels of a lysosomal protein by modulating the translocon complex. The translocon is a complex of proteins associated with the translocation of proteins across the cell membranes, e.g., the complex that transports nascent polypeptides with a targeting signal sequence into the interior (cisternal or lumenal) space of the endoplasmic reticulum (ER) from the cytosol. This translocation process requires the protein to cross a hydrophobic lipid bilayer. The same complex is also used to integrate nascent proteins into the membrane itself (membrane proteins). The translocon complex is a hetero-trimeric protein complex called Sec61. It comprises the subunits Sec61α, Sec61β, and Sec61γ. Sec61α is the large pore subunit. During protein translocation, the channel rearranges to move an alpha-helix “plug” out of the way, and a polypeptide chain is moved from the cytoplasmic funnel, through the pore ring, the extracellular funnel, into the extracellular space. Proteins destined to be secreted elongate through the Sec61 channel and get N-glycosylated on asparagine residues by OST before finally having their signal peptide cleaved by the signal peptidase.

Levels of one or more lysosomal proteins can be increased by contacting the translocon complex with an agent that modulates the translocon complex thereby increasing the lysosomal protein level. The level of lysosomal protein can be increased intracellularly and/or the secretion of the lysosomal protein can be increased such that extracellular levels are increased.

### Increasing Lysosomal Protein Levels

It has been discovered that by modulating the activity of the translocon complex, the level of lysosomal protein can be increased. Without being bound to any particular theory, it is hypothesized that the amount of lysosomal protein is increased by one of four mechanisms, or any combination of these four mechanisms.

Increased trafficking of the lysosomal protein can be due to increased translation of mRNA sub-population, translocation facilitation into the endoplasmic reticulum, increased glycosylation of the lysosomal protein thereby increasing its stability, and/or enhanced trafficking of the lysosomal protein via the transmembrane proteins mannose-6-phosphate receptor (M6PR) and/or sortilin.

The compounds disclosed herein can be used in methods of treating a disorder associated with low levels of a lysosomal protein by administering to a subject suffering therefrom a disclosed compound that modulates the translocon complex thereby increasing amounts of the lysosomal protein and treat the disorder.

The lysosomal protein level can be increased, compared to a control, by at least 5%, at least 10%, at least 25%, at least 30%, at least 40%, at least 50%, at least 75%, at least 90%, at least 100%, at least 125%, at least 150%, or at least 200%.

Amount of lysosomal protein can be assessed using typical biological assays, including those as described in the examples section below. The level of lysosomal protein measured can be intracellular, extracellular (i.e., secreted protein), or a combination of each. In some cases, the level of lysosomal protein secreted from a cell is increased (e.g., the extracellular protein levels are increased). In some cases, the level of intracellular lysosomal protein is increased.

Provided herein are methods of increasing the levels of a lysosomal protein in a subject by administering a compound as disclosed herein. The lysosomal protein can be progranulin, prosaposin, β-glucocerebrosidase, galactosidase alpha, cathepsin B, cathepsin Z, neuraminidase 1, tripeptidyl peptidase, alpha-L-fucosidase 2, mannosidase alpha class 2B member 2, mannosidase beta, serine carboxypeptidase 1, acid ceramidase, GM2 ganglioside activator, cathepsin D, cathepsin S, cathepsin K, cathepsin L, or hexosaminidase. Increasing the levels of a lysosomal protein can impact a disorder associated with aberrant levels of the lysosomal protein. For example, the disorder can be a lysosomal storage disorder, a neurodegenerative disease, an inflammatory disease, or a disease selected from stroke, Down syndrome, congenital heart disease, diabetes, common variable immune deficiency (CVID), tubulo-interstitial kidney disease (TKD), polycystic liver disease, myocarditis, dermatitis hyperhomocysteinemia, endo-toxic shock, lung injury, bone defect (e.g., inflammatory periodontal bone defect), or osteolysis.

In various cases, the subject suffers from a lysosomal storage disorder. The lysosomal storage disorder can be mucopolysaccharidosis, sphingolipidosis, glycogen storage disease type II, glycoprotein storage disease, Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, mucopolysaccharidosis-plus syndrome, Fabry disease, Gaucher disease, Tay-Sachs disease, sialidosis, Niemann Pick type A, Niemann Pick type B, galactosialidosis, Niemann pick type C, I-cell disease, mucolipidosis type III, GM1 gangliosidosis, β-galactosidase deficiency, α-mannosidosis, GM2 gangliosidosis, β-mannosidosis, Krabbe, fucosidosis, metachromatic leukodystrophy, aspartylglucosaminuria, multiple sulfatase deficiency, Schindler, Farber lipogranulomatosis, Pompe disease, Wolman disease, Danon disease, free sialic acid storage disease, ceroid lipofuscinosis, β-glucuronidase hypoactivity disease, Sandhoff disease, or cholesterol ester storage disease.

In various cases, the disorder is a neurodegenerative disease. The neurodegenerative disease can be Parkinson's disease (e.g., Parkinson's disease with GBA mutation), frontotemporal dementia, Alzheimer's disease, Huntington's disease, traumatic brain injury, neuronal ceroid lipofuscinosis (NCL), multiple sclerosis, amyotrophic lateral sclerosis (ALS), aigyrophilic grain dementia, Alexander's disease, Alper's disease, cerebral palsy, Cockayne syndrome, corticobasal degeneration, Creutzfeldt-Jakob disease, dementia pugilistica, diffuse neurofibrillary tangles with calcification, HIV-associated dementia, Lewy body dementia, Kennedy's disease, neuroborreliosis, primary lateral sclerosis, Refsum's disease, Gerstmann-Straussler-Scheinker disease, Hallevorden-Spatz disease, hereditary diffuse leukoencepholopathy with spheroids (HDLS), inclusion body myositis, multiple system atrophy, myotonic dystrophy, Nasu-Hakola disease, Schilder's disease, Wobbly Hedgehog Syndrome (WHS), Duchenne-Aran muscular atrophy, progressive bulbar palsy, pseudobulbar palsy, HIV-associated neurocognitive disorder (HAND), tauopathy, chronic traumatic encephalopathy, or cerebellar downbeat nystagmus.

In various cases, the disorder is an inflammatory disease. The inflammatory disease can be Sjogren disease, inflammatory arthritis, osteoarthritis, inflammatory bowel disease, or immune thrombocytopenia.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

### Synthesis of Compounds Disclosed Herein

Compounds can be synthesized using typical synthetic chemistry techniques using commercially available starting materials, compounds known in the literature, or from readily prepared intermediates, by employing standard synthetic methods and procedures either known to those of skill in the art, or in light of the teachings herein. Generally, the synthesis of the disclosed compounds can be achieved following similar syntheses as detailed in Schemes A and B below and in the Examples.

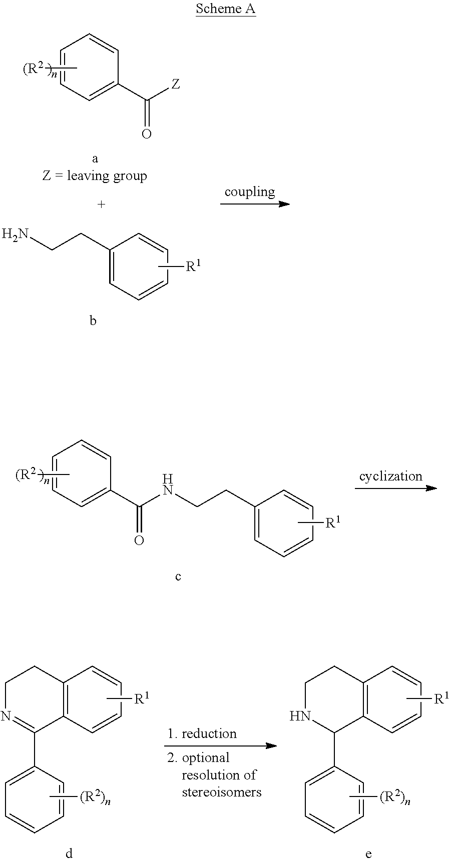

Compounds having structure e can be synthesized using the procedure shown in Scheme A. For example, reaction of an optionally substituted benzoic acid derivative having structure a with an optionally substituted 2-phenylethan-1-amine b produces optionally substituted N-phenethylbenzamide compounds having structure c. Cyclization under appropriate conditions gives optionally substituted 1-phenyl-3,4-dihydroisoquinoline compounds having structure d. Subsequent reduction followed by optional isolation of single stereoisomers by resolution or chromatographic means gives substituted tetrahydroquinoline compounds having structure e.

The coupling of compounds a and b can be catalyzed by appropriate reagents selected based on the precise nature of compounds a and b. For example, when compound a is an acid chloride compound (i.e., when Z is Cl), the coupling of compounds a and b can be catalyzed by e.g., triethylamine. Compounds a and b can be purchased commercially or prepared by a variety of methods from commercially-available starting materials.

Cyclization of compound c can be effected with the use of various reactions known in the art. For example, the cyclization can involve an acid-catalyzed electrophilic aromatic substitution reaction, e.g., cyclization under Bischler-Napieralski reaction conditions. For example, c can be cyclized by treatment with triflic anhydride in the presence of e.g., chloropyridine in a solvent, e.g., dichloromethane. Alternately, compound c can be cyclized by treatment with polyphosphoric acid (PPA).

Compound d can be reduced to form compound e with or without asymmetric induction of a stereocenter. For example, compound d can be treated with a reducing agent, e.g., sodium borohydride, in a solvent, e.g., methanol. Reduction of compound d can be followed by the formation of a desired stereoisomer, e.g., by crystallization in the presence of D-tartaric acid. Alternately, compound d can be reduced via asymmetric hydrogenation to directly produce substituted tetrahydroquinoline compound e as the desired stereoisomer. For example, compound d can be reduced with H₂ gas in the presence of an iridium catalyst, such as [{Ir(H)[(S,S)-(f)-binaphane]}₂(μ-I);]⁺I⁻.

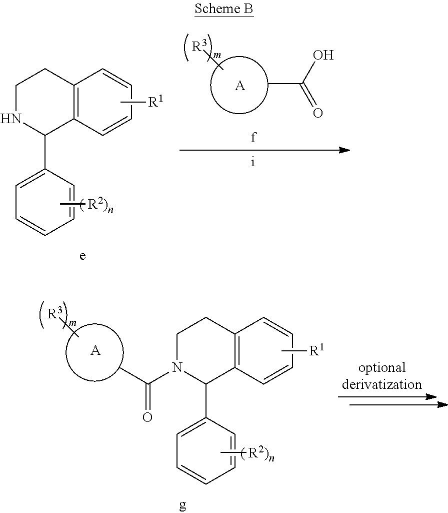

Compounds described herein, e.g., compounds of Formula I, can be synthesized from compounds of structure e using the procedures shown in Scheme B. For example, reaction of a compound having structure e with an alcohol or amine compound having structure f under coupling conditions i produces a substituted tetrahydroquinolinyl compound having structure g. The coupling can be facilitated by an appropriate reagent, such as a carbodiimide reagent. Subsequent optional derivatization gives compounds as disclosed herein, e.g., compounds of Formula (I).

Appropriate coupling conditions i for the reaction between compounds e and f are known to those skilled in the art. For example, the reaction can be carried out using carbodiimide coupling conditions, e.g., EDC/HOBt, or other peptide coupling conditions e.g., (1-[bis(dimethylamino)methylene]-1H-1,2,3-triazolo[4,5-b]pyridinium 3-oxide hexafluorophosphate (HATU) and a base such as N,N-diisopropylethylamine (DIPEA) or trimethylamine (TEA), i.e., HATU/DIPEA or HATU/TEA, in an organic solvent, e.g., dichloromethane (DCM) or dimethylformamide (DMF).

### EXAMPLES

### General Methods

LCMS: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-1000, ELSD Alltech 3300 gas flow 1.5 ml/min, gas temp: 40° C., Eluent A: 0.1% formic acid in acetonitrile, Eluent B: 0.1% formic acid in water).

Method A: column: Waters XSelect™ C18, 30×2.1 mm, 3.5μ, Temp: 35° C., Flow: 1 mL/min, Gradient: t₀=5% A, t_{1.6 min}=98% A, t_{3 min}=98% A, Posttime: 1.3 min.

Method C: column: Waters XSelect™ C18, 50×2.1 mm, 3.5μ, Temp: 35° C., Flow: 0.8 mL/min, Gradient: t₀=5% A, t_{3.5 min}=98% A, t_{6 min}=98% A, Posttime: 2 min.

Method AK: column: Waters XSelect™ CSH C18, 50×2.1 mm, 3.5μ, Temp: 40° C., Flow: 0.8 mL/min, Gradient: t₀=5% A, t_{4.5 min}=98% A, t_{6 min}=98% A, Posttime: 2 min.

Method BV: column: Waters XSelect™ C18, 30×2.1 mm, 3.5μ, Temp: 25° C., Flow: 1 mL/min, Gradient: t₀=5% A, t_{1.6 min}=98% A, t_{4 min}=98% A, Posttime: 1.3 min.

LCMS: Apparatus: Agilent 1260 Bin. Pump: G1312B, degasser; autosampler, ColCom, DAD: Agilent G1315C, 220-320 nm, MSD: Agilent LC/MSD G6130B ESI, pos/neg 100-1000, Eluent A: acetonitrile, Eluent B: 10 mM ammoniumbicarbonate in water (pH=9.5).

Method B: column: Waters XSelect™ CSH C18, 30×2.1 mm, 3.5μ, Temp: 25° C., Flow: 1 mL/min, Gradient: t₀=5% A, t_{1.6 min}=98% A, t_{3 min}=98% A, Posttime: 1.3 min.

Method D: column: Waters XSelect™ CSH C18, 50×2.1 mm, 3.5μ, Temp: 25° C., Flow: 0.8 mL/min, Gradient: t₀=5% A, t_{3.5 min}=98% A, t_{6 min}=98% A, Posttime: 2 min.

Method AQ: column: Waters XSelect™ CSH C18, 50×2.1 mm, 3.5μ, Temp: 25° C., Flow: 0.8 mL/min, Gradient: t₀=5% A, t_{4.5} min=98% A, t_{6 min}=98% A, Posttime: 2 min.

Method J: column: Phenomenex GeminiNX C18, 50×2.0 mm, 3μ, Temp: 25° C., Flow: 0.8 mL/min, Gradient: t₀=5% A, t_{3.5 min}=98% A, t_{6 min}=98% A, Posttime: 2 min.

LCMS: Apparatus: Agilent 1290 series with UV detector (220 nm, 270 nm (band width 100 nm)), and HP 6130 MSD mass detector (API-ES positive and negative).

Method E: column: Waters XBridge BEH XP (2.1×50 mm; 2.5 μm; 1034 bar), Temp: 35° C., Flow: 0.6 mL/min, t₀=80% A, t_{1.5} min=0% A, t_{3 min}=0% A. Eluent A: 100% water, Eluent B: 100% methanol/acetonitrile 1:1.

Method O: column: Waters XBridge BEH XP (2.1×50 mm; 2.5 μm; 1034 bar), Temp: 35° C., Flow: 0.6 mL/min, t₀=100% A, t_{1.5} min=50% A, t_{2 min}=20% A. Eluent A: 0.05% trifluoroacetic acid in water, Eluent B: 100% acetonitrile.

Method K: column: Waters XBridge BEH XP (2.1×50 mm; 2.5 μm; 1034 bar), Temp: 35° C., Flow: 0.6 mL/min, t₀=80% A, t_{1.5 min}=0% A, t_{4 min}=0% A. Eluent A: ammonium acetate (10 mM); water/methanol/acetonitrile (90:6:4), Eluent B: ammonium acetate (10 mM); water/methanol/acetonitrile (10:54:36).

LCMS: Apparatus: Agilent Infinity II; Bin. Pump: G7120A, Multisampler, VTC, DAD: Agilent G7117B, 220 and 220-320 nm, PDA: 210-320 nm, MSD: Agilent G6135B ESI, pos/neg 100-1000, ELSD G7102A: Evap 40° C., Neb 40° C., gasflow 1.6 ml/min.

Method P: column: Waters XSelect CSH C18, 50×2.1 mm, 2.5 μm, Temp: 40° C., Flow: 0.6 mL/min, Gradient: t₀=5% A, t_{2 min}=98% A, t_{2.7 min}=98% A, Posttime: 0.3 min, Eluent A: 0.1% formic acid in acetonitrile, Eluent B: 0.1% formic acid in water.

Method Q: column: Waters XSelect CSH C18, 50×2.1 mm, 2.5 μm, Temp: 25° C., Flow: 0.6 mL/min, Gradient: t₀=5% A, t_{2 min}=98% A, t_{2.7 min}=98% A, Posttime: 0.3 min, Eluent A: acetonitrile, Eluent B: 10 mM ammonium bicarbonate in water (pH=9.5).

Method AH: column: XBridge Shield RP C18 (50×2.1 mm 2.5 μm), Temp: 25° C., Flow: 0.6 mL/min, Gradient: t₀=5% A, t_{2 min}=98% A, t_{2.7 min}=98% A, Posttime: 0.3 min, Eluent A: 100 mM ammonia in acetonitrile, Eluent B: 100 mM ammonia in water (pH=10).

Method BB: column: Waters XBridge BEH Shield RP (50×2.1 mm; 2.5 μm), Temp: 40° C., Flow: 0.6 mL/min, t₀=5% A, t_{2.0 min}=98% A, t_{2.7 min}=98% A, Posttime: 0.3 min, Eluent A: 0.1% formic acid in acetonitrile, Eluent B: 0.1% formic acid in water.

Method CA: column: Waters XSelect CSH C18, 50×2.1 mm, 2.5 μm, Temp: 40° C., Flow: 0.8 mL/min, Gradient: t₀=5% A, t_{0.5}=5% A, t_{4.5 min}=98% A, t_{5 min}=98% A, Posttime: 0.5 min, Eluent A: 0.1% formic acid in acetonitrile, Eluent B: 0.1% formic acid in water.

Method CB: column: Waters XSelect CSH C18, 50×2.1 mm, 2.5 μm, Temp: 25° C., Flow: 0.8 mL/min, Gradient: t₀=5% A, t_{0.5 min}=5% A, t_{4.5 min}=98% A, t_{5 min}=98% A, Posttime: 0.5 min, Eluent A: acetonitrile, Eluent B: 10 mM ammonium bicarbonate in water (pH=9.5).

LCMS: Apparatus: Waters Acquity UPLC H-Class with PDA detector and SQD mass detector (API-ES positive and negative.

Method R: column: Waters XBridge BEH C18 (2.1×50 mm; 2.5 μm), Temp: 30° C., Flow: 0.6 mL/min, t₀=80% A, t_{1.5 min}=5% A, t_{2.5 min}=5% A, Posttime: 0.5 min, Eluent A: 10 mM ammonium acetate in water with 5% acetonitrile, Eluent B: acetonitrile.

Method BI: column: Waters Acquity CSH C18, 100×2.1 mm, 1.7 μm, Temp: 40° C., Flow: 0.45 mL/min, Gradient: t₀=5% A, t_{5.0 min}=98% A, t_{6.0 min}=98% A, Posttime: 0.5 min, Eluent A: 0.1% formic acid in acetonitrile, Eluent B: 0.1% formic acid in water.

LCMS: Apparatus: Waters Iclass; Bin. Pump: UPIBSM, SM: UPISMFTN with SO; UPCMA, PDA: UPPDATC, 210-320 nm, SQD: ACQ-SQD2 ESI; ELSD: gaspressure 40 psi, drift tube temp: 50° C.

Method AV: column: Waters Acquity Shield RP18 (50×2.1 mm; 1.7 μm), Temp: 25° C., Flow: 0.5 mL/min, t₀=5% B, t_{2.0 min}=98% B, t_{2.7} min=98% B, Posttime: 0.3 min, Eluent A: 10 mM ammonium bicarbonate in water (pH=9.5), Eluent B: acetonitrile.

Chiral LC:

Apparatus: Agilent 1260 Quat. Pump: G1311C, degasser; autosampler, ColCom, DAD: Agilent G1315D (210 nm, 220 nm, 220-320 nm).

Method H: column: Chiralcel OD-H (250×4.6 mm, 5 μm); Column temp: 25° C.; flow: 1.0 mL/min; isocratic gradient of 0.1% diethylamine in heptane/isopropanol 95/05.

Method I: column: Chiralcel OD-H (250×4.6 mm, 5 μm); Column temp: 25° C.; flow: 1.0 mL/min; isocratic gradient of 0.1% diethylamine in heptane/ethanol 80/20.

Method L: column: Chiralcel OD-H (250×4.6 mm, 5 μm); Column temp: 25° C.; flow: 1.0 mL/min; isocratic gradient of 0.1% diethylamine in heptane/isopropanol 90/10.

Method AB: column: Chiralpak AD-H (250×4.6 mm, 5 μm); Column temp: 25° C., flow: 0.8 mL/min, Isocratic gradient of 0.1% diethylamine in heptane/ethanol 30/70.

Method AC: column: Chiralpak AD-H (250×4.6 mm, 5 μm); Column temp: 25° C., flow: 1.0 mL/min, Isocratic gradient of 0.1% diethylamine in heptane/ethanol 70/30.

Method AG: column: Chiralpak AD-H (250×4.6 mm, 5 μm); Column temp: 25° C., flow: 1.0 mL/min, Isocratic gradient of heptane/isopropanol 70/30.

SFC: Apparatus: Waters Acquity UPC²: Waters ACQ-ccBSM Binary Pump; Waters ACQ-CCM Convergence Manager; Waters ACQ-SM Sample Manager—Fixed Loop; Waters ACQ-CM Column Manager-30S; Waters ACQ-PDA Photodiode Array Detector (210-400 nm); Waters ACQ-ISM Make Up Pump, Waters Acquity QDa MS Detector (pos 100-650).

Method F: column: Phenomenex Cellulose-2 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

Method BL: column: Phenomenex Cellulose-2 (100×4.6 mm, 5 μm), Temp: 35° C., BPR: 170 bar, Flow: 2.5 mL/min, Gradient: t₀=10% B, t_{5 min}=40% B, t_{6 min}=40% B, Posttime: 0.5 min; Eluent A: CO₂, Eluent B: 20 mM ammonia in 2-propanol

Method G: column: Phenomenex Amylose-1 (100×4.6 mm, 5 μm), Temp: 35° C., BPR: 170 bar, Flow: 2.5 mL/min, Gradient: t₀=5% B, t_{2.5 min}=50% B, t_{10 min}=50% B, Posttime: 0.5 min; Eluent A: CO₂, Eluent B: 20 mM ammonia in methanol

Method W: column: Phenomenex Amylose-1 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

Method X: column: Phenomenex Amylose-1 (100×4.6 mm, 5 μm), Temp: 35° C., BPR: 170 bar, Flow: 2.5 mL/min, Gradient: t₀=5% B, t_{5 min}=30% B, t_{6 min}=30% B, Posttime: 0.5 min; Eluent A: CO₂, Eluent B: 20 mM ammonia in methanol

Method N: column: Diacel Chiralpak IG-3 (3.0×150 mm, 3 μm), Temp: 40° C., BPR: 126 bar, Flow: 2.0 mL/min, Pump program: 30% B isocratic, Eluent A: CO₂, Eluent B: 0.2% ammonia in methanol

Method V: column: Phenomenex Cellulose-1 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

Method AO: column: Phenomenex Cellulose-1 (100×4.6 mm, 5 μm), Temp: 35° C., BPR: 170 bar, Flow: 2.5 mL/min, Gradient: t₀=2% B, t_{5 min}=10% B, t_{6 min}=10% B, Posttime: 0.5 min; Eluent A: CO₂, Eluent B: 20 mM ammonia in methanol

Method BM: column: Phenomenex Cellulose-1 (100×4.6 mm, 5 μm), Temp: 35° C., BPR: 170 bar, Flow: 2.5 mL/min, Gradient: t₀=10% B, t_{5 min}=40% B, t_{6 min}=40% B, Posttime: 0.5 min; Eluent A: CO₂, Eluent B: 20 mM ammonia in methanol

Method AA: column: Phenomenex Lux Cellulose 4 (3.0×150 mm, 3 μm); Temp: 40° C., BPR: 138 bar; Flow: 2.5 mL/min, Gradient: t₀=2% B, t_{4 min}=27% B, Posttime: 1 min; Eluent A: CO₂, Eluent B: methanol

Method AD: column: Diacel Chiralpak IC (4.6×100 mm, 5 μm); Temp: 35° C., BPR: 170 bar; Flow: 2.5 mL/min, Gradient: t₀=5% B, t_{5 min}=50% B, Posttime: 0.5 min; Eluent A: CO₂, Eluent B: 20 mM ammonia in methanol

Method A1: column: Diacel Chiralpak IC (4.6×100 mm, 5 μm); Temp: 35° C., BPR: 170 bar; Flow: 2.5 mL/min, Gradient: t₀=5% B, t_{5 min}=50% B, Posttime: 0.5 min; Eluent A: CO₂, Eluent B: 20 mM ammonia in 2-propanol

Method AR: column: Waters Acquity UPC2 BEH (3.0×100 mm, 1.7 μm); Temp: 35° C., BPR: 210 bar; Flow: 1.0 mL/min, Gradient: t₀=2% B, t_{4 min}=20% B, t_{6 min}=20% B, Posttime: 0.5 min; Eluent A: CO₂, Eluent B: 20 mM ammonia in methanol

Method AS: column: Waters Acquity UPC2 BEH (3.0×100 mm, 1.7 μm); Temp: 35° C., BPR: 210 bar; Flow: 1.0 mL/min, Gradient: t₀=2% B, t_{4 min}=50% B, t_{6 min}=50% B, Posttime: 0.5 min; Eluent A: CO₂, Eluent B: 20 mM ammonia in methanol

Method AU: column: Waters Acquity UPC2 Torus 2-PIC (100×3.0 mm 1.7 μm); Temp: 35° C., BPR: 170 bar; Flow: 1.0 mL/min, Gradient: t₀=2% B, t_{4 min}=50% B, t_{6 min}=50% B, Posttime: 0.5 min; Eluent A: CO₂, Eluent B: 20 mM ammonia in methanol

Method AZ: column: Phenomenex iAmylose-3 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

Method BZ: column: Phenomenex iAmylose-3 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in ethanol

Method BE: column: Phenomenex Cellulose-1 (100×4.6 mm, 5 μm), Temp: 35° C., BPR: 120 bar, Flow: 2.5 mL/min, Gradient: t₀=10% B, t_{5 min}=30% B, t_{6 min}=30% B, Posttime: 0.5 min; Eluent A: CO₂, Eluent B: 20 mM ammonia in methanol

GCMS1: Instrument: GC: Agilent 6890N and MS: 5973 MSD, El-positive, Det.temp.: 280° C. Mass range: 50-550; Column: RXi-5MS 20 m, ID 180 μm, df 0.18 μm; Average velocity: 50 cm/s; Injection vol: 1 μl; Injector temp: 250° C.; Split ratio: 100/1; Carrier gas: He;

Method A20: Initial temp: 100° C.; Initial time: 1.5 min; Solvent delay: 1.0 min; Rate 75° C./min; Final temp 250° C.; Final time 3.5 min.

Method SC_S20: Initial temp: 60° C.; Initial time: 1.0 min; Solvent delay: 1.3 min; Rate 50° C./min; Final temp 250° C.; Final time 3.5 min.

Preparative Methods

Basic reversed phase MPLC: Instrument type: Reveleris™ prep MPLC; Eluent A: 99% acetonitrile+1% 10 mM ammoniumbicarbonate in water (pH=9.0); Eluent B: 10 mM ammoniumbicarbonate in water (pH=9.0). Unless otherwise stated, column: Waters XSelect CSH C18 (145×25 mm, 10p); Flow: 40 mL/min; Column temp: room temperature, was used. Otherwise Column: Phenomenex Gemini C18 (185×25 mm, 10p); Flow: 40 mL/min; Column temp: room temperature.

Acidic reversed phase MPLC: Instrument type: Reveleris™ prep MPLC; Column: Phenomenex LUNA C18(3) (150×25 mm, 10p); Flow: 40 mL/min; Column temp: room temperature; Eluent A: 0.1% (v/v) Formic acid in acetonitrile, Eluent B: 0.1% (v/v) Formic acid in water.

Preparative SFC: Apparatus: Waters Prep 100 SFC UV/MS directed system; Waters 2998 Photodiode Array (PDA) Detector; Waters Acquity QDa MS detector; Waters 2767 Sample Manager. Eluent A: CO₂, Eluent B: 20 mM ammonia in methanol. Eluent C: 20 mM ammonia in 2-propanol. Eluent D: 20 mM ammonia in ethanol.

Method S: Column: Phenomenex Lux Cellulose-2 (250×21.2 mm, 5 μm); Column temp: 35° C.; flow: 70 mL/min; ABPR: 120 bar; Linear gradient: t=0 min 10% B, t=6 min 50% B; Detection: PDA (210-400 nm)/TIC.

Method T: Column: Phenomenex Lux Amylose-1 (250×21 mm, 5 μm); Column temp: 35° C.; flow: 70 mL/min; ABPR: 120 bar; Linear gradient: t=0 min 5% B, t=3 min 10% B; t=8.5 min 10% B; Detection: PDA (210-400 nm)/TIC.

Method Y: Column: Phenomenex Lux Amylose-1 (250×21 mm, 5 μm); Column temp: 35° C.; flow: 70 mL/min; ABPR: 120 bar; Linear gradient: t=0 min 10% B, t=6 min 40% B; t=7.5 min 40% B; Detection: PDA (210-400 nm)/TIC.

Method Z: Column: Phenomenex Lux Amylose-1 (250×21 mm, 5 μm); Column temp: 35° C.; flow: 70 mL/min; ABPR: 120 bar; Linear gradient: t=0 min 10% B, t=6 min 50% B; t=7.5 min 50% B; Detection: PDA (210-400 nm)/TIC.

Method AW: Column: Phenomenex Lux Amylose-1 (250×21 mm, 5 μm); Column temp: 35° C.; flow: 70 mL/min; ABPR: 120 bar; Linear gradient: t=0 min 5% B, t=6 min 50% B; t=7.5 min 50% B; Detection: PDA (210-400 nm)/TIC.

Method U: Column: Phenomenex Lux Cellulose-1 (250×21.2 mm, 5 μm); Column temp: 35° C.; flow: 70 mL/min; ABPR: 120 bar; Linear gradient: t=0 min 10% B, t=6.5 min 30% B, t=8 min 30% B; Detection: PDA (210-400 nm)/TIC.

Method AE: Column: Diacel Chiralpak IC for SFC (250×20 mm, 5 μm); Column temp: 35° C.; Flow: 70 ml/min; ABPR: 120 bar; Linear gradient: t=0 min 10% B, t=6 min 40% B, t=7.5 min 40% B; Detection: PDA (210-400 nm); Fraction collection: PDA TIC.

Method AL: Column: Phenomenex Lux Cellulose-2 (250×21.2 mm, 5 μm); Column temp: 35° C.; flow: 70 mL/min; ABPR: 120 bar; Linear gradient: t=0 min 30% B, t=6 min 50% B; t=7.5 min 50% B; Detection: PDA (210-400 nm); Fraction collection: PDA TIC.

Method AM: Column: Diacel Chiralpak IC for SFC (250×20 mm, 5 μm); Column temp: 35° C.; Flow: 70 ml/min; ABPR: 120 bar; Linear gradient: t=0 min 25% B, t=6 min 50% B; Detection: PDA (210-400 nm); Fraction collection: PDA TIC.

Method AN: Column: Diacel Chiralpak IC for SFC (250×20 mm, 5 μm); Column temp: 35° C.; Flow: 70 ml/min; ABPR: 120 bar; Linear gradient: t=0 min 10% B, t=6 min 50% B, t=7.5 min 50% B; Detection: PDA (210-400 nm); Fraction collection: PDA TIC.

Method AP: Column: Diacel Chiralpak IC for SFC (250×20 mm, 5 μm); Column temp: 35° C.; Flow: 70 ml/min; ABPR: 120 bar; Linear gradient: t=0 min 10% C, t=6 min 50% C, t=7.5 min 50% C; Detection: PDA (210-400 nm); Fraction collection: PDA TIC.

Method AT: Column: Waters Torus 2-PIC 130A OBD (250×19 mm, 5 μm); Column temp: 35° C.; Flow: 70 ml/min; ABPR: 120 bar; Linear gradient: t=0 min 10% B, t=4 min 50% B, t=7 min 50% B; Detection: PDA (210-400 nm); Fraction collection: PDA TIC.

Method BA: Column: Diacel Chiralpak IC for SFC (250×20 mm, 5 μm); Column temp: 35° C.; Flow: 70 ml/min; ABPR: 120 bar; Linear gradient: t=0 min 50% B, t=6 min 50% B, t=7.5 min 50% B; Detection: PDA (210-400 nm); Fraction collection: PDA TIC.

Method BD: Column: Phenomenex Lux Cellulose-1 (250×21.2 mm, 5 μm); Column temp: 35° C.; flow: 70 mL/min; ABPR: 120 bar; Linear gradient: t=0 min 10% B, t=6.5 min 40% B, t=8 min 40% B; Detection: PDA (210-400 nm)/TIC.

Method BG: Column: Phenomenex Lux Cellulose-2 (250×21.2 mm, 5 μm); Column temp: 35° C.; flow: 70 mL/min; ABPR: 120 bar; Linear gradient: t=0 min 20% B, t=6 min 30% B; Detection: PDA (210-400 nm)/TIC.

Method BH: Column: Waters Torus 2-PIC 130A OBD (250×19 mm, 5 μm); Column temp: 35° C.; Flow: 70 ml/min; ABPR: 120 bar; Linear gradient: t=0 min 5% B, t=4 min 30% B, t=7 min 30% B; Detection: PDA (210-400 nm); Fraction collection: PDA TIC.

Method BK: Column: Phenomenex Lux Cellulose-2 (250×21.2 mm, 5 μm); Column temp: 35° C.; flow: 70 mL/min; ABPR: 120 bar; Linear gradient: t=0 min 10% C, t=6 min 40% C; Detection: PDA (210-400 nm)/TIC.

Method BN: Column: Diacel Chiralpak IC for SFC (250×20 mm, 5 μm); Column temp: 35° C.; Flow: 70 ml/min; ABPR: 120 bar; Linear gradient: t=0 min 35% B, t=6 min 50% B; Detection: PDA (210-400 nm); Fraction collection: PDA TIC.

Method BO: Column: Phenomenex Lux Amylose-1 (250×21 mm, 5 μm); Column temp: 35° C.; flow: 70 mL/min; ABPR: 120 bar; Linear gradient: t=0 min 50% B, t=4 min 50% B; Detection: PDA (210-400 nm)/TIC.

Method BP: Column: Diacel Chiralpak IC for SFC (250×20 mm, 5 μm); Column temp: 35° C.; Flow: 70 ml/min; ABPR: 120 bar; Linear gradient: t=0 min 20% B, t=3.5 min 30% B; Detection: PDA (210-400 nm); Fraction collection: PDA TIC.

Method BQ: Column: Phenomenex Lux iAmylose-3 (250×21 mm, 5 μm); Column temp: 35° C.; Flow: 70 ml/min; ABPR: 120 bar; Linear gradient: t=0 min 10% B, t=5 min 50% B, t=7 min 50% B; Detection: PDA (210-400 nm); Fraction collection: PDA TIC.

Method BR: Column: Phenomenex Lux Cellulose-1 (250×21.2 mm, 5 μm); Column temp: 35° C.; flow: 70 mL/min; ABPR: 120 bar; Linear gradient: t=0 min 10% B, t=6.5 min 50% B, t=8 min 50% B; Detection: PDA (210-400 nm)/TIC.

Method BS: Column: Waters Viridis BEH Prep OBD (250×19 mm, 5 μm); Column temp: 35° C.; Flow: 70 ml/min; ABPR: 120 bar; Linear gradient: t=0 min 2% B, t=5 min 50% B; Detection: PDA (210-400 nm); Fraction collection based on PDA TIC.

Method BT: Column: Phenomenex Lux Cellulose-2 (250×21.2 mm, 5 μm); Column temp: 35° C.; flow: 70 mL/min; ABPR: 120 bar; Linear gradient: t=0 min 5% B, t=6 min 50% B; Detection: PDA (210-400 nm)/TIC.

Method BW: Column: Waters Viridis BEH Prep OBD (250×19 mm, 5 μm); Column temp: 35° C.; Flow: 70 ml/min; ABPR: 120 bar; Isocratic method: 10% B for 8 min; Detection: PDA (210-400 nm); Fraction collection based on PDA TIC.

Method BX: Column: Phenomenex Lux iAmylose-3 (250×21 mm, 5 μm); Column temp: 35° C.; Flow: 70 ml/min; ABPR: 120 bar; Linear gradient: t=0 min 5% C, t=5 min 50% C, t=7 min 50% C; Detection: PDA (210-400 nm); Fraction collection: PDA TIC.

Method BY: Column: Phenomenex Lux iAmylose-3 (250×21 mm, 5 μm); Column temp: 35° C.; Flow: 70 ml/min; ABPR: 120 bar; Linear gradient: t=0 min 5% D, t=5 min 50% D, t=7 min 50% D; Detection: PDA (210-400 nm); Fraction collection: PDA TIC.

Preparative Chiral HPLC: Apparatus: Shimadzu LC8-A preparative pumps, Shimadzu SCL-10Avp system controller, Shimadzu SPD-10Avp UV-VIS detector; Fraction Collector: Gilson 215 Liquid Handler.

Method AF: Column: Diacel Chiralpak AD-H, 20×250 mm, 5 μm, Flow: 18 mL/min, isocratic heptane/isopropanol. time: 60 min, Eluent A: 70%, Eluent B: 30%.

Method AJ: Column: Diacel Chiralpak AD-H, 20×250 mm, 5 μm, Flow: 18 mL/min, isocratic 0.1% diethylamine in heptane/ethanol. time: 60 min, Eluent A: 30%, Eluent B: 70%.

Method AY: Column: Diacel Chiralcel OD, 20×250 mm, 10 μm, Flow: 18 mL/min, isocratic heptane/ethanol. time: 60 min, Eluent A: 90%, Eluent B: 10%.

Preparative LCMS: Apparatus: Agilent Technologies 1290 preparative LC; MS instrument type: Agilent Technologies G6130B Quadrupole; Detection: DAD (220-320 nm); MSD (ESI pos/neg) mass range: 100-800; fraction collection based on MS and/or DAD.

Method M: column: Xbridge Amide (150×19 mm, 5p); flow: 25 mL/min; column temp: room temperature; Eluent A: 100% acetonitrile; Eluent B: 10 mM ammonium bicarbonate in water (pH=9.0).

Preparative LCMS: Agilent Technologies G6130B Quadrupole; HPLC instrument type: Agilent Technologies 1200 preparative LC; Detection: DAD (220-320 nm); Detection: MSD (ESI pos/neg) mass range: 100-1000; Fraction collection based on MS and DAD.

Method AX: column: Xbridge Amide (150×19 mm, 5p); flow: 25 mL/min; column temp: room temperature; Eluent A: 100% acetonitrile; Eluent B: 10 mM ammonium bicarbonate in water (pH=9.0). lin. gradient: t=0 min 2% A, t=2.5 min 2% A, t=11 min 30% A, t=13 min 100% A, t=17 min 100% A.

Method BC: column: Waters XSelect CSH (C18, 100×30 mm, 10p); Flow: 25 ml/min; Column temp: RT; Eluent A: 0.1% formic acid in acetonitrile; Eluent B: 0.1% formic acid in water; lin. gradient: t=0 min 2% A, t=2 min 2% A, t=8.5 min 30% A, t=10 min 100% A, t=13 min 100% A.

Method BU: column: Waters XBridge Shield (C18, 150×19 mm, 5p); Flow: 25 ml/min; Column temp: RT; Eluent A: 10 mM ammonium bicarbonate in water pH=9.5; Eluent B: 100% acetonitrile; lin. gradient: t=0 min 20% B, t=2.5 min 20% B, t=11 min 60% B, t=13 min 100% B, t=17 min 100% B.

Preparative LCMS: Agilent Technologies G6120AA Quadrupole; HPLC instrument type: Agilent Technologies 1200 preparative LC; Detection: DAD (220-320 nm); Detection: MSD (ESI pos/neg) mass range: 100-1000; Fraction collection based on MS and DAD.

Method BF: column: Waters XSelect CSH (C18, 100×30 mm, 10 μm); Flow: 55 ml/min; Column temp: RT; Eluent A: 100% acetonitrile; Eluent B: 0.1% formic acid in water; lin. gradient: t=0 min 30% A, t=2 min 30% A, t=8.5 min 70% A, t=10 min 100% A, t=13 min 100% A.

Preparative LCMS: MS instrument type: ACQ-SQD2; HPLC instrument type: Waters Modular Preparative HPLC System. Detection: DAD (220-320 nm); Detection: MSD (ESI pos/neg) mass range: 100-800; Fraction collection based on MS and DAD.

Method BJ: column: Waters Xselect (C18, 100×30 mm, 10 μm); flow: 55 mL/min; Column temp: RT; Eluent A: 10 mM ammonium bicarbonate in water pH=9.5, eluent B: 100% acetonitrile; lin. gradient: t=0 min 5% B, t=2.5 min 5% B, t=4 min 20% B, t=13 min 60% B, t=14.5 100% B, t=17 min 100% B.

¹H-NMR:

400 MHz ¹H-NMR spectra were recorded on a Bruker Avance-400 ultrashield NMR spectrometer, using CDCl₃ or DMSO-d6 as solvent and are reported in ppm using TMS (0.00 ppm) as an internal standard.

300 MHz ¹H-NMR spectra were recorded on a Varian VNMRS: 7.05 Tesla magnet from Oxford Instruments. Chemical shifts are denoted in 5 (ppm) and are referenced to the residual protic solvent.

All CP Analytical-SFC experiments were run on SFC Method Station (Thar, Waters), Column temperature: 40° C., Mobile phase: CO₂/Methanol (or Ethanol or isopropanol) containing 0.2% MA (MA=7M Methanolic Ammonia) Flow: 4.0 ml/min, Back Pressure: 120 Bar, Detection wavelength: 214 nm;

All CP Analytical-chiral HPLC experiments were run on Agilent-1200 (Agilent), Column temperature: 40° C., Mobile phase: n-Hexane (0.1% DEA)/Ethanol (0.1% DEA). Flow rate: 1.0 ml/min, Detection wavelength: 214 nm&254 nm.

Preparative Methods:

All CP Preparative-SFC experiments were run on SFC-80 (Thar, Waters), Column temperature: 35° C., Mobile phase: CO₂/Methanol (or Ethanol or isopropanol) containing 0.2% MA (MA=7M Methanolic Ammonia). Flow rate: 80 g/min, Back pressure: 100 bar, Detection wavelength: 214 nm.

All CP Preparative-chiral HPLC experiments were run on Gilson-281 (Gilson), Column temperature: 40° C., Mobile phase: n-Hexane (0.1% DEA)/Ethanol (0.1% DEA). Flow rate: 50 ml/min, Detection wavelength: 214 nm.

Lcms Experiments:

All CP LCMS experiments were run on Agilent 1200, with a column temperature of 40° C., monitoring UV absorption at 214 nm and scanning a mass range from 100-1000. Individual conditions vary slightly as described in the methods below:

LCMS CP Method A: Column: ZORBAX SB-C18 3.0*50 mm, 3.5 μm; Mobile Phase: A: Water (0.1% TFA), B: ACN (0.1% TFA); Gradient: 5% B increase to 95% B over 1.3 min, stop at 3 min. Flow Rate: 1.8 mL/min

LCMS CP Method B: Column: XBridge C18 50*4.6 mm, 3.5 μm; Mobile Phase: A: Water (0.1% TFA), B: ACN (0.1% TFA); Gradient: 5% B increase to 95% B over 1.2 min, stop at 3 min. Flow Rate: 2.0 mL/min

LCMS CP Method C: Column: XBridge SB-C18 3.0*50 mm, 3.5 μm; Mobile Phase: A: Water (10 mM NH₄HCO₃), B: ACN; Gradient: 5% B increase to 95% B over 1.2 min. Flow Rate: 2.0 mL/min;

LCMS CP Method C1: Column: XBridge SB-C18 3.0*50 mm, 3.5 μm; Mobile Phase: A: Water (10 mM NH₄HCO₃), B: ACN; Gradient: 5% B increase to 95% B over 1.4 min. Flow Rate: 2.0 mL/min;

LCMS CP Method C2: Column: SunFire-C18 4.6*50 mm, 3.5 μm; Mobile Phase: A: Water (10 mM NH₄HCO₃), B: ACN; Gradient: 5% B increase to 95% B over 1.4 min. Flow Rate: 2.0 mL/min;

LCMS CP Method D: Column: SunFire-C18 3.0*50 mm, 3.5 μm; Mobile Phase: A: Water (0.01% TFA), B: ACN (0.01% TFA); Gradient: 5% B increase to 95% B over 1.3 min, stop at 3 min. Flow Rate: 2.0 mL/min;

LCMS CP Method E: Column: XBridge SB-C18 3.0*50 mm, 3.5 μm; Mobile Phase: A: Water (0.1% TFA), B: ACN (0.1% TFA); Gradient: 5% B increase to 95% B over 1.8 min, stop at 3 min. Flow Rate: 1.8 mL/min;

LCMS CP Method F: Column: XBridge C18 4.6*50 mm, 3.5 μm; Mobile Phase: A: Water (0.1% TFA), B: ACN (0.1% TFA); Gradient: 5% B increase to 95% B over 1.7 min, stop at 3 min. Flow Rate: 1.8 mL/min

### Experimental Procedures

### Example 1

### Synthesis of ((2R,5S)-5-(aminomethyl)tetrahydrofuran-2-yl)((S)-1-(4-fluorophenyl)-3,4-dihydroisoquinolin-2(1H)-yl)methanone (Compound 5000) and ((2S,5R)-5-(aminomethyl)tetrahydrofuran-2-yl)((S)-1-(4-fluorophenyl)-3,4-dihydroisoquinolin-2(1H)-yl)methanone (Compound 5001)

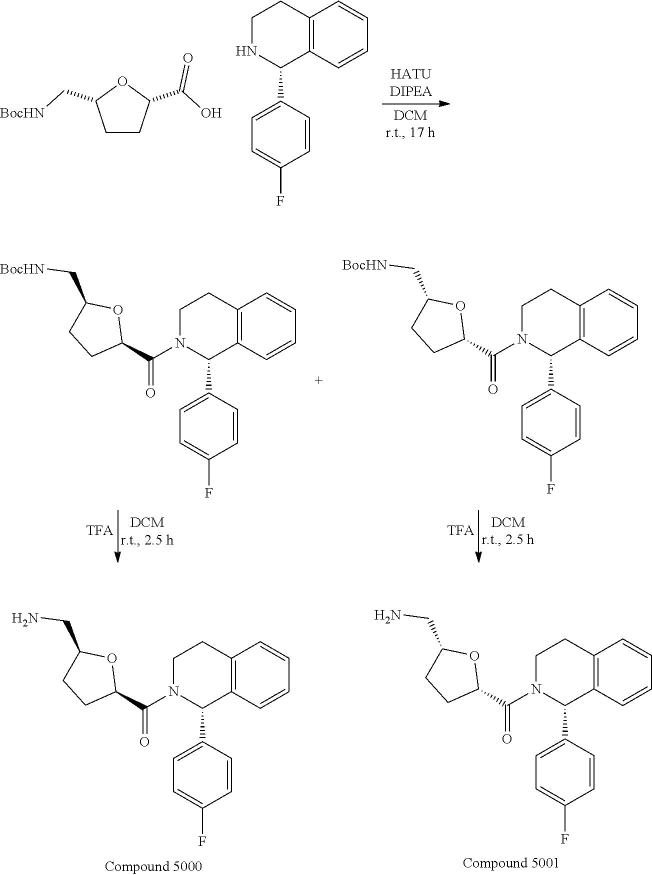

### Synthesis of tert-butyl (((2S,5R)-5-((S)-1-(4-fluorophenyl)-1,2,3,4-tetrahydroisoquinoline-2-carbonyl)tetrahydrofuran-2-yl)methyl)carbamate and tert-butyl (((2R,5S)-5-((S)-1-(4-fluorophenyl)-1,2,3,4-tetrahydroisoquinoline-2-carbonyl)tetrahydrofuran-2-yl)methyl)carbamate

Starting from cis-5-(((tert-butoxycarbonyl)amino)methyl)tetrahydrofuran-2-carboxylic acid (97.4 mg, 0.397 mmol) and (S)-1-(4-fluorophenyl)-1,2,3,4-tetrahydroisoquinoline (98.6 mg, 0.434 mmol), tert-butyl (((2S,5R)-5-((S)-1-(4-fluorophenyl)-1,2,3,4-tetrahydroisoquinoline-2-carbonyl)tetrahydrofuran-2-yl)methyl)carbamate (51 mg, 28%) as the first eluting isomer and tert-butyl (((2R,5S)-5-((S)-1-(4-fluorophenyl)-1,2,3,4-tetrahydroisoquinoline-2-carbonyl)tetrahydrofuran-2-yl)methyl)carbamate as the second eluting isomer were prepared according to the procedure described for (Sand R)-5-((S)-1-(4-fluorophenyl)-1,2,3,4-tetrahydroisoquinoline-2-carbonyl)dihydrofuran-3(2H)-one (see Compound 5004), additional acidic workup (aqueous HCl (1 M)/dichloromethane), and purification by preparative chiral SFC (method S). Absolute configuration of the cis-tetrahydropyran-2-carboxamide centers was arbitrarily assigned.

First eluting isomer: SFC: RT=2.89 min. (M+Na)⁺=472 (method F). Synthesis of ((2R,5S)-5-(aminomethyl)tetrahydrofuran-2-yl)((S)-1-(4-fluorophenyl)-3,4-dihydroisoquinolin-2(1H)-yl)methanone (Compound 5000).

Starting from tert-butyl (((2S,5R)-5-((S)-1-(4-fluorophenyl)-1,2,3,4-tetrahydroisoquinoline-2-carbonyl)tetrahydrofuran-2-yl)methyl)carbamate (51 mg, 0.112 mmol), ((2R,5S)-5-(aminomethyl)tetrahydrofuran-2-yl)((S)-1-(4-fluorophenyl)-3,4-dihydroisoquinolin-2(1H)-yl)methanone (Compound 5000) was prepared according to the procedure described for Compound 5008 after basic workup (saturated aqueous NaHCO₃/dichloromethane extraction) and lyophilization from acetonitrile and water (1:1).

LCMS: 98%, RT=1.06 min. (M+H)⁺=355 (method P). Synthesis of ((2S,5R)-5-(aminomethyl)tetrahydrofuran-2-yl)((S)-1-(4-fluorophenyl)-3,4-dihydroisoquinolin-2(1H)-yl)methanone (Compound 5001).

Starting from tert-butyl (((2R,5S)-5-((S)-1-(4-fluorophenyl)-1,2,3,4-tetrahydroisoquinoline-2-carbonyl)tetrahydrofuran-2-yl)methyl)carbamate (35 mg, 0.077 mmol), ((2S,5R)-5-(aminomethyl)tetrahydrofuran-2-yl)((S)-1-(4-fluorophenyl)-3,4-dihydroisoquinolin-2(1H)-yl)methanone (Compound 5001) was prepared according to the procedure described for Compound 5008 after basic workup (saturated aqueous NaHCO₃/dichloromethane extraction) and lyophilization from acetonitrile and water (1:1).

LCMS: 99%, RT=1.07 min. (M+H)⁺=355 (method P).

### Example 2

### Synthesis of ((2R,5R)-5-(aminomethyl)tetrahydrofuran-2-yl)((S)-1-(4-fluorophenyl)-3,4-dihydroisoquinolin-2(1H)-yl)methanone (Compound 5002)

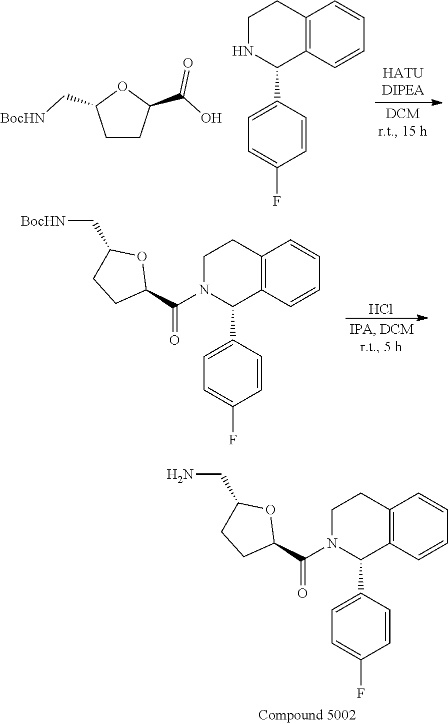

### Synthesis of tert-butyl (((2R,5R)-5-((S)-1-(4-fluorophenyl)-1,2,3,4-tetrahydroisoquinoline-2-carbonyl)tetrahydrofuran-2-yl)methyl)carbamate

Starting from (2R,5R)-5-(((tert-butoxycarbonyl)amino)methyl)tetrahydrofuran-2-carboxylic acid (synthesis according to methods reported in: Tetrahedron Lett., 2014, 55, 3569 and J. Org. Chem., 2010, 65, 6441; 0.11 g, 0.44 mmol) and (S)-1-(4-fluorophenyl)-1,2,3,4-tetrahydroisoquinoline (0.11 g, 0.48 mmol), tert-butyl (((2R,5R)-5-((S)-1-(4-fluorophenyl)-1,2,3,4-tetrahydroisoquinoline-2-carbonyl)tetrahydrofuran-2-yl)methyl)carbamate was prepared according to the procedure described for (S and R)-5-((S)-1-(4-fluorophenyl)-1,2,3,4-tetrahydroisoquinoline-2-carbonyl)dihydrofuran-3(2H)-one (see Compound 5004).

LCMS: 84%, RT=2.31 min., (M+H)⁺=455 (method K).

### Synthesis of ((2R,5R)-5-(aminomethyl)tetrahydrofuran-2-yl)((S)-1-(4-fluorophenyl)-3,4-dihydroisoquinolin-2(1H)-yl)methanone (Compound 5002)

HCl (6 M in 2-propanol, 0.25 mL, 1.5 mmol) was added to a solution of tert-butyl (((2R,5R)-5-((S)-1-(4-fluorophenyl)-1,2,3,4-tetrahydroisoquinoline-2-carbonyl)tetrahydrofuran-2-yl)methyl)carbamate (53 mg, 0.12 mmol) in 2-propanol (1 mL) at room temperature. After stirring the reaction mixture for 6 hours, additional HCl (6 M in 2-Propanol, 0.25 mL, 1.5 mmol) was added stirring was continued for 72 hours. Then, the reaction mixture was diluted with aqueous K₂CO₃ (2 M, 10 mL) and extracted with dichloromethane (2×25 mL). The combined organic layer was dried over Na₂SO₄ and evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm) (product isolated by dichloromethane extraction from basified (aqueous saturated NaHCO₃) fractions) and lyophilized from a mixture of acetonitrile and water (1:1)). ((2R,5R)-5-(aminomethyl)tetrahydrofuran-2-yl)((S)-1-(4-fluorophenyl)-3,4-dihydroisoquinolin-2(1H)-yl)methanone (Compound 5002).

LCMS: 99%, RT=1.05 min., (M+H)⁺=355 (method P).

### Example 3

### Synthesis of ((2S,5S)-5-(aminomethyl)tetrahydrofuran-2-yl)((S)-1-(4-fluorophenyl)-3,4-dihydroisoquinolin-2(1)-yl)methanone (Compound 5003)

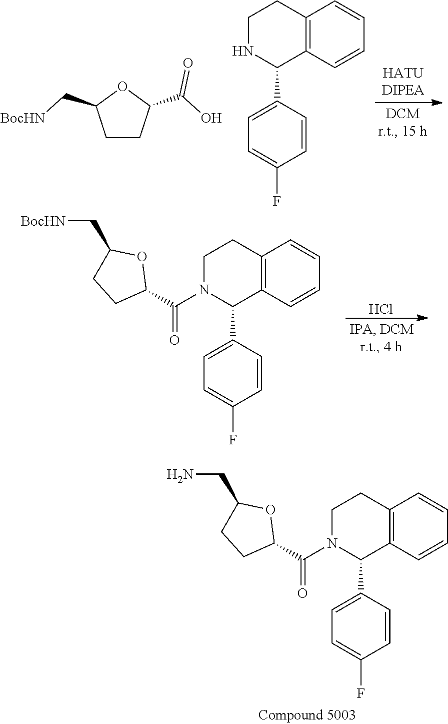

### Synthesis of tert-butyl (((2S,5S)-5-((S)-1-(4-fluorophenyl)-1,2,3,4-tetrahydroisoquinoline-2-carbonyl)tetrahydrofuran-2-yl)methyl)carbamate

Starting from (2S,5S)-5-(((tert-butoxycarbonyl)amino)methyl)tetrahydrofuran-2-carboxylic acid (synthesis according to methods reported in: Tetrahedron Lett., 2014, 55, 3569, using (R,R)-salenCo(II) as catalyst and J. Org. Chem., 2010, 65, 6441; 0.13 g, 0.54 mmol) and (S)-1-(4-fluorophenyl)-1,2,3,4-tetrahydroisoquinoline (0.14 g, 0.59 mmol), tert-butyl (((2S,5S)-5-((S)-1-(4-fluorophenyl)-1,2,3,4-tetrahydroisoquinoline-2-carbonyl)tetrahydrofuran-2-yl)methyl)carbamate was prepared according to the procedure described for (Sand R)-5-((S)-1-(4-fluorophenyl)-1,2,3,4-tetrahydroisoquinoline-2-carbonyl)dihydrofuran-3(2H)-one (see Compound 5004).

LCMS: 81%, RT=2.30 min., (M+H)⁺=455 (method K). Synthesis of ((2S,5S)-5-(aminomethyl)tetrahydrofuran-2-yl)((S)-1-(4-fluorophenyl)-3,4-dihydroisoquinolin-2(1H)-yl)methanone (Compound 5003).

Starting from tert-butyl (((2S,5S)-5-((S)-1-(4-fluorophenyl)-1,2,3,4-tetrahydroisoquinoline-2-carbonyl)tetrahydrofuran-2-yl)methyl)carbamate (66 mg, 0.15 mmol), ((2S,5S)-5-(aminomethyl)tetrahydrofuran-2-yl)((S)-1-(4-fluorophenyl)-3,4-dihydroisoquinolin-2(1H)-yl)methanone (Compound 5003) was prepared according to the procedure described for Compound 5002 and purified by acidic preparative MPLC (Linear Gradient: t=0 min 5% A; t=1 min 5% A; t=2 min 10% A; t=17 min 50% A; t=18 min 100%; t=23 min 100% A; detection: 220 nm) (product isolated by dichloromethane extraction from basified (aqueous saturated NaHCO₃) fractions) and lyophilized from a mixture of acetonitrile and water (1:1)).

LCMS: 98%, RT=1.04 min., (M+H)⁺=355 (method P).

### Example 4

### Synthesis of ((2S,4R)-4-(aminomethyl)-4-hydroxytetrahydrofuran-2-yl)((S)-1-(4-fluorophenyl)-3,4-dihydroisoquinolin-2(1H)-yl)methanone (Compound 5004), ((2S,4S)-4-(aminomethyl)-4-hydroxytetrahydrofuran-2-yl)((S)-1-(4-fluorophenyl)-3,4-dihydroisoquinolin-2(1H)-yl)methanone (Compound 5005), ((2R,4S)-4-(aminomethyl)-4-hydroxytetrahydrofuran-2-yl)((S)-1-(4-fluorophenyl)-3,4-dihydroisoquinolin-2(1H)-yl)methanone (Compound 5006), and ((2R,4R)-4-(aminomethyl)-4-hydroxytetrahydrofuran-2-yl)((S)-1-(4-fluorophenyl)-3,4-dihydroisoquinolin-2(1H)-yl)methanone (Compound 5007)

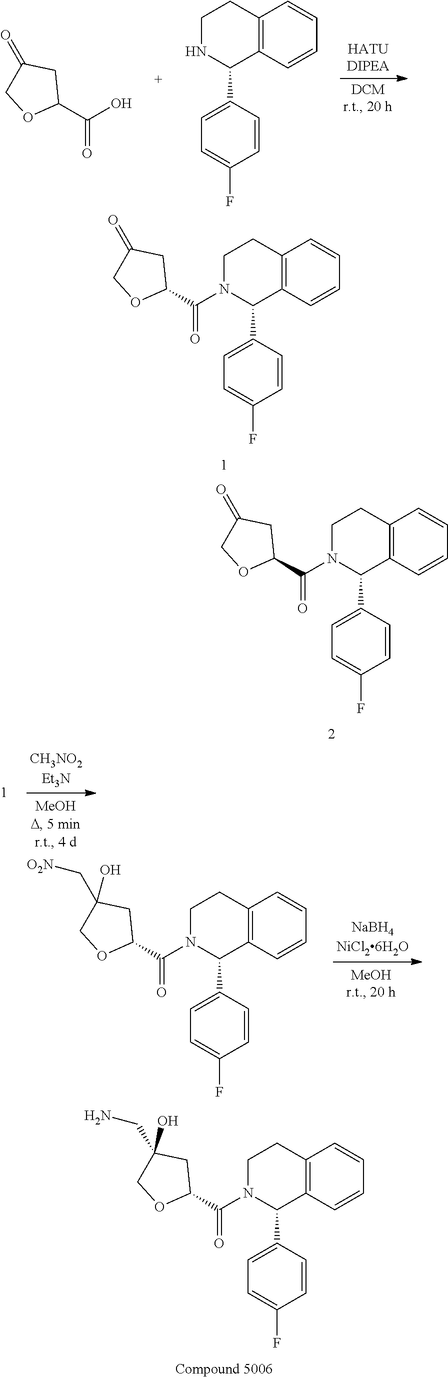

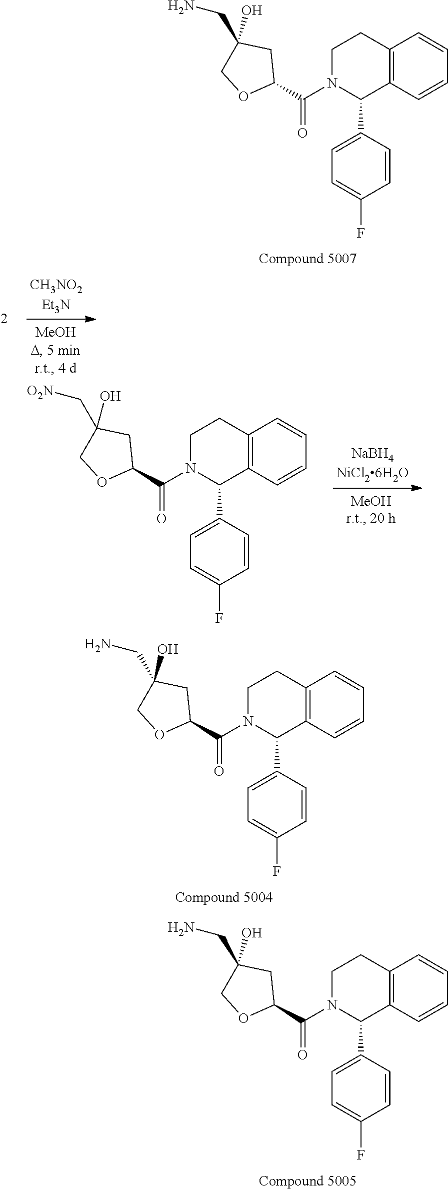 [SMILES: NC[C@]1(O)CO[C@@H](C(=O)N2CCc3ccccc3[C@@H]2c2ccc(F)cc2)C1 ; O=C([C@@H]1CC(O)(C[N+](=O)[O-])CO1)N1CCc2ccccc2[C@@H]1c1ccc(F)cc1 ; NC[C@@]1(O)CO[C@H](C(=O)N2CCc3ccccc3[C@@H]2c2ccc(F)cc2)C1 ; NC[C@]1(O)CO[C@H](C(=O)N2CCc3ccccc3[C@@H]2c2ccc(F)cc2)C1]

### Synthesis of (R)-5-((S)-1-(4-fluorophenyl)-1,2,3,4-tetrahydroisoquinoline-2-carbonyl)dihydrofuran-3(2H)-one and (S)-5-((S)-1-(4-fluorophenyl)-1,2,3,4-tetrahydroisoquinoline-2-carbonyl)dihydrofuran-3(2H)-one

N,N-diisopropylethylamine (3.83 mL, 22.0 mmol) and 1-[bis(dimethylamino)methylene]-1H-1,2,3-triazolo[4,5-b]pyridinium 3-oxide hexafluorophosphate (HATU, 3.54 g, 9.3 mmol) were added to a solution of 4-oxotetrahydrofuran-2-carboxylic acid (1.1 g, 8.46 mmol) and (S)-1-(4-fluorophenyl)-1,2,3,4-tetrahydroisoquinoline (1.92 g, 8.46 mmol) in dichloromethane (40 mL). After stirring at room temperature for 20 hours, the reaction mixture was washed with saturated aqueous NaHCO₃ (3×50 mL), dried over Na₂SO₄, and evaporated under reduced pressure. The residue was purified by flash column chromatography (silica, 0 to 30% ethyl acetate in heptane) to give (R)-5-((S)-1-(4-fluorophenyl)-1,2,3,4-tetrahydroisoquinoline-2-carbonyl)dihydrofuran-3(2H)-one as the first eluting isomer and (S)-5-((S)-1-(4-fluorophenyl)-1,2,3,4-tetrahydroisoquinoline-2-carbonyl)dihydrofuran-3(2H)-one as the second eluting isomer. Absolute configuration of the tetrahydropyran-2-carboxamide center was determined by X-ray crystallography.

First eluting isomer: LCMS: 95%, RT=2.04 min., (M+H)⁺=340 (method K).

Second eluting isomer: LCMS 95%, RT=2.03 min., (M+H)⁺=340 (method K).

### Synthesis of ((S)-1-(4-fluorophenyl)-3,4-dihydroisoquinolin-2(1H)-yl)((2S)-4-hydroxy-4-(nitromethyl)tetrahydrofuran-2-yl)methanone

A mixture of (S)-5-((S)-1-(4-fluorophenyl)-1,2,3,4-tetrahydroisoquinoline-2-carbonyl)dihydrofuran-3(2H)-one (0.22 g, 0.65 mmol), nitromethane (300 mg, 4.91 mmol) and triethylamine (2.6 mL, 19 mmol) in methanol (3 mL) was heated to reflux for 5 minutes and then stirred at room temperature for 4 days. The reaction mixture was evaporated to dryness under reduced pressure (at 60° C.) and the residue purified by flash column chromatography (silica, 0 to 50% ethyl acetate in heptane) to give ((S)-1-(4-fluorophenyl)-3,4-dihydroisoquinolin-2(1H)-yl)((2S)-4-hydroxy-4-(nitromethyl)tetrahydrofuran-2-yl)methanone which was used as such.

LCMS: 44:54 mixture of isomers at RT=2.00 min. and 2.04 min., (M+H)⁺=401 (method K).

### Synthesis of ((S)-1-(4-fluorophenyl)-3,4-dihydroisoquinolin-2(1H)-yl)((2R)-4-hydroxy-4-(nitromethyl)tetrahydrofuran-2-yl)methanone

Starting from (R)-5-((S)-1-(4-fluorophenyl)-1,2,3,4-tetrahydroisoquinoline-2-carbonyl)dihydrofuran-3(2H)-one (0.20 g, 0.59 mmol), ((S)-1-(4-fluorophenyl)-3,4-dihydroisoquinolin-2(1H)-yl)((2R)-4-hydroxy-4-(nitromethyl)tetrahydrofuran-2-yl)methanone was prepared according to the procedure described for ((S)-1-(4-fluorophenyl)-3,4-dihydroisoquinolin-2(1H)-yl)((2S)-4-hydroxy-4-(nitromethyl)tetrahydrofuran-2-yl)methanone (vide supra) and used directly in the next step.

LCMS: 48:51 mixture of isomers at RT=2.02 min. and 2.05 min., (M+H)⁺=401 (method K).

### Synthesis of ((2S,4S)-4-(aminomethyl)-4-hydroxytetrahydrofuran-2-yl)((S)-1-(4-fluorophenyl)-3,4-dihydroisoquinolin-2(1H)-yl)methanone (Compound 5005) and ((2S,4R)-4-(aminomethyl)-4-hydroxytetrahydrofuran-2-yl)((S)-1-(4-fluorophenyl)-3,4-dihydroisoquinolin-2(1H)-yl)methanone (Compound 5004)

NaBH₄ (0.13 g, 3.5 mmol) was added portionwise to a solution of ((S)-1-(4-fluorophenyl)-3,4-dihydroisoquinolin-2(1H)-yl)((2S)-4-hydroxy-4-(nitromethyl)tetrahydrofuran-2-yl)methanone (70 mg, 0.17 mmol) and NiCl₂·6H₂O (21 mg, 0.087 mmol) in methanol (15 mL) at room temperature resulting in an initial vigorous exotherm. After stirring at room temperature for 20 hours, saturated aqueous NaHCO₃ (25 mL) was added and stirring was continued for 30 minutes. Ethyl acetate (50 mL) was added and the mixture was thoroughly mixed. Brine (20 mL) was added and the mixture was thoroughly mixed. The layers were separated and the aqueous phase was extracted with ethyl acetate (2×30 mL). The combined organic phase was dried overNa₂SO₄ and concentrated under reduced pressure. The residue was purified by preparative chiral SFC (method Z) and the product containing fractions lyophilized from a mixture of acetonitrile and water (1:1, 4 mL) to give ((2S,4S)-4-(aminomethyl)-4-hydroxytetrahydrofuran-2-yl)((S)-1-(4-fluorophenyl)-3,4-dihydroisoquinolin-2(1H)-yl)methanone (Compound 5005) as the first eluting isomer and ((2S,4R)-4-(aminomethyl)-4-hydroxytetrahydrofuran-2-yl)((S)-1-(4-fluorophenyl)-3,4-dihydroisoquinolin-2(1H)-yl)methanone (Compound 5004) as the second eluting isomer. The absolute configuration of the tetrahydropyran-2-carboxamide was determined by X-ray crystallography and tetrahydropyran-4-hydroxy-aminomethyl centers were arbitrarily assigned.

Compound 5005: LCMS: 98%, RT=2.54 min., (M+H)⁺=371 (method AK). SFC: 100%, RT=4.16 min., (M+H)⁺=371 (method W).

Compound 5004: LCMS: 95%, RT=2.50 min., (M+H)⁺=371 (method AK). SFC: d.e.=96.5%, RT=4.90 min., (M+H)⁺=371 (method W). Synthesis of ((2R,4R)-4-(aminomethyl)-4-hydroxytetrahydrofuran-2-yl)((S)-1-(4-fluorophenyl)-3,4-dihydroisoquinolin-2(1H)-yl)methanone (Compound 5007) and ((2R,4S)-4-(aminomethyl)-4-hydroxytetrahydrofuran-2-yl)((S)-1-(4-fluorophenyl)-3,4-dihydroisoquinolin-2(1H)-yl)methanone (Compound 5006).

Starting from ((S)-1-(4-fluorophenyl)-3,4-dihydroisoquinolin-2(1H)-yl)((2R)-4-hydroxy-4-(nitromethyl)tetrahydrofuran-2-yl)methanone (130 mg, 0.325 mmol), ((2R,4R)-4-(aminomethyl)-4-hydroxytetrahydrofuran-2-yl)((S)-1-(4-fluorophenyl)-3,4-dihydroisoquinolin-2(1H)-yl)methanone (Compound 5007) as the first eluting isomer and ((2R,4S)-4-(aminomethyl)-4-hydroxytetrahydrofuran-2-yl)((S)-1-(4-fluorophenyl)-3,4-dihydroisoquinolin-2(1/H)-yl)methanone (Compound 5006) as the second eluting isomer were prepared according to the procedure described for ((2S,4S and 2S,4R)-4-(aminomethyl)-4-hydroxytetrahydrofuran-2-yl)((S)-1-(4-fluorophenyl)-3,4-dihydroisoquinolin-2(1H)-yl)methanone (vide supra) and purified by preparative chiral SFC (method AN). Absolute configuration was determined by X-ray crystallography.

Compound 5007: LCMS: 97%, RT=2.49 min., (M+H)⁺=371 (method AK). SFC: 100%, RT=3.72 min., (M+H)⁺=371 (method AD).

Compound 5006: LCMS: 99%, RT=2.53 min., (M+H)⁺=371 (method AK). SFC: d.e.=97.5%, RT=4.16 min., (M+H)⁺=371 (method AD).

### Example 5

### Synthesis of ((2S,4S)-4-(aminomethyl)tetrahydrofuran-2-yl)((S)-1-(4-fluorophenyl)-3,4-dihydroisoquinolin-2(1H)-yl)methanone (Compound 5008) and ((2S,4R)-4-(aminomethyl)tetrahydrofuran-2-yl)((S)-1-(4-fluorophenyl)-3,4-dihydroisoquinolin-2(1H)-yl)methanone (Compound 5009)

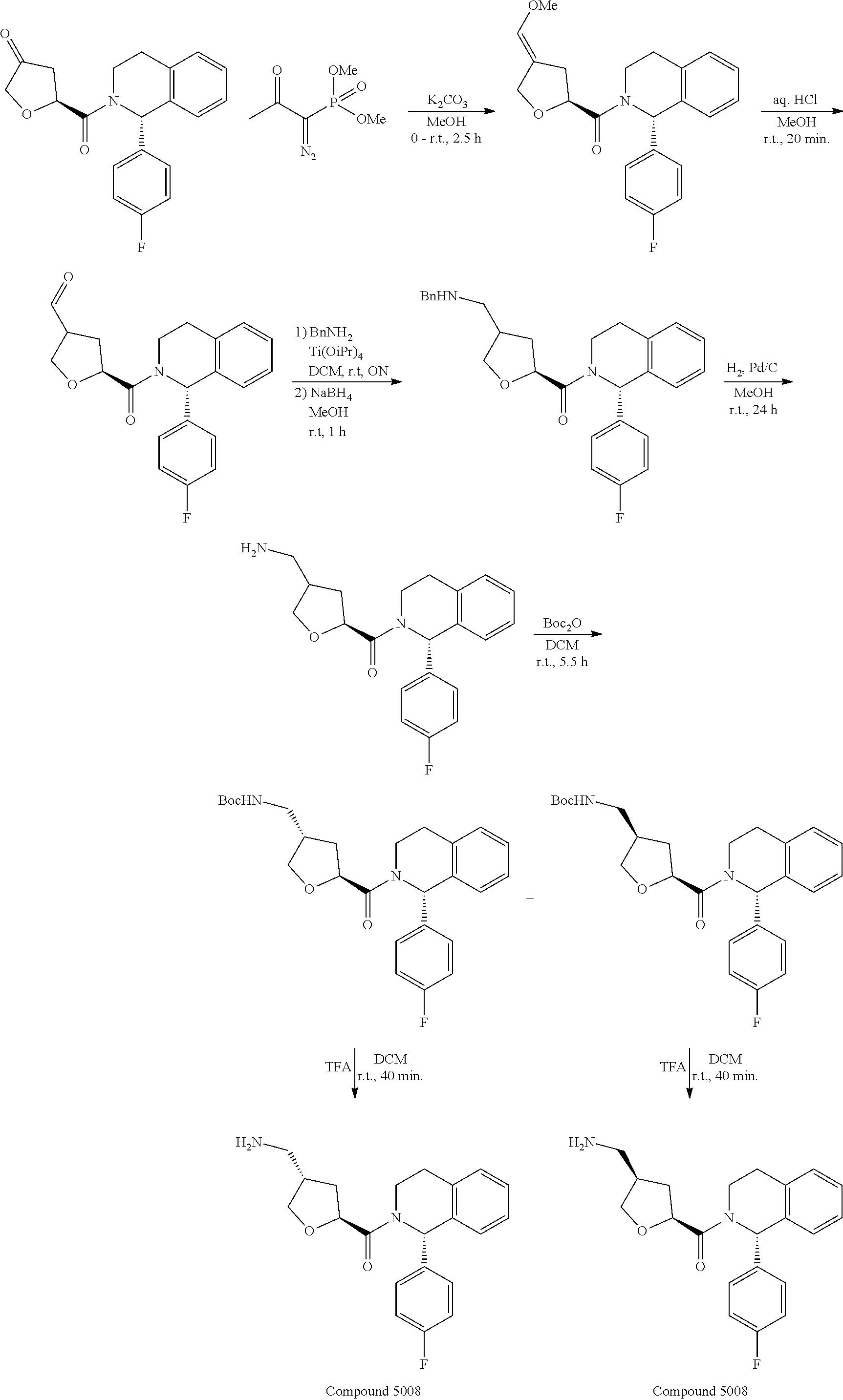

### Synthesis of ((S)-1-(4-fluorophenyl)-3,4-dihydroisoquinolin-2(1H)-yl)((S)-4-(methoxymethylene)tetrahydrofuran-2-yl)methanone

At 0° C., dimethyl (1-diazo-2-oxopropyl)phosphonate (0.5 g, 2.6 mmol) and K₂CO₃ (512 mg, 3.7 mmol) were added to a solution of (S)-5-((S)-1-(4-fluorophenyl)-1,2,3,4-tetrahydroisoquinoline-2-carbonyl)dihydrofuran-3(2H)-one (see Compound 5004, 300 mg, 0.88 mmol) in methanol (8 mL). The reaction mixture was allowed to warm to room temperature. After 2.5 hours, the mixture was diluted with water (15 mL) and ethyl acetate (50 mL) and the aqueous phase was saturated with NaCl. The layers were separated and the aqueous phase was extracted with ethyl acetate (2×50 mL). The combined organic phase was dried over Na₂SO₄ and evaporated under reduced pressure to give ((S)-1-(4-fluorophenyl)-3,4-dihydroisoquinolin-2(1H)-yl)((S)-4-(methoxymethylene)tetrahydrofuran-2-yl)methanone

LCMS: RT=2.20 min., (M+H)⁺=368 (method K).

### Synthesis of (5S)-5-((S)-1-(4-fluorophenyl)-1,2,3,4-tetrahydroisoquinoline-2-carbonyl)tetrahydrofuran-3-carbaldehyde

Aqueous HCl (2.0 M, 2.5 mL, 5.0 mmol) was added to a solution of ((S)-1-(4-fluorophenyl)-3,4-dihydroisoquinolin-2(1H)-yl)((S)-4-(methoxymethylene)tetrahydrofuran-2-yl)methanone (325 mg, 0.884 mmol) in acetonitrile (5 mL). After 20 minutes, the reaction mixture was diluted with water (40 mL) and extracted with ethyl acetate (2×50 mL). The combined organic phase dried over Na₂SO₄ and evaporated to dryness under reduced pressure to give (5S)-5-((S)-1-(4-fluorophenyl)-1,2,3,4-tetrahydroisoquinoline-2-carbonyl)tetrahydrofuran-3-carbaldehyde which was directly used in the next step.

LCMS: RT=2.00 min, (M+H)⁺=354 (method K).

### Synthesis of ((2S)-4-((benzylamino)methyl)tetrahydrofuran-2-yl)((S)-1-(4-fluorophenyl)-3,4-dihydroisoquinolin-2(1H)-yl)methanone

Benzylamine (1.0 g, 9.3 mmol) followed by titanium(IV) isopropoxide (1.0 mL, 3.5 mmol) were added to a solution of crude (5S)-5-((S)-1-(4-fluorophenyl)-1,2,3,4-tetrahydroisoquinoline-2-carbonyl)tetrahydrofuran-3-carbaldehyde (0.884 mmol) in dichloromethane (3 mL). The reaction mixture was stirred at room temperature overnight and then evaporated to dryness under reduced pressure. The residue was taken up in methanol (30 mL) and NaBH₄ (981 mg, 25.9 mmol) was added portion wise. The mixture was stirred for 1 hour before aqueous NaOH (0.1 M, 25 mL) was added and the mixture extracted with methyl tert-butyl ether (3×50 mL). The combined organic layers were evaporated under reduced pressure (at 60° C.) and the residue was coevaporated from ethanol (2×20 mL) to give ((2S)-4-((benzylamino)methyl)tetrahydrofuran-2-yl)((S)-1-(4-fluorophenyl)-3,4-dihydroisoquinolin-2(1H)-yl)methanone which was directly used in the next step.

LCMS: RT=2.14 min., (M+H)⁺=445 (method K).

### Synthesis of ((2S)-4-(aminomethyl)tetrahydrofuran-2-yl)((S)-1-(4-fluorophenyl)-3,4-dihydroisoquinolin-2(1H)-yl)methanone

Palladium (10% on activated carbon, 200 mg, 0.188 mmol) was added to a solution of ((2S)-4-((benzylamino)methyl)tetrahydrofuran-2-yl)((S)-1-(4-fluorophenyl)-3,4-dihydroisoquinolin-2(1H)-yl)methanone (380 mg, 0.855 mmol) in methanol (40 mL). The mixture was stirred under a hydrogen atmosphere for 24 hours then flushed with nitrogen, filtered over Celite, and washed with methanol (2×40 mL). The combined filtrates were evaporated under reduced pressure (at 65° C.). The residue was purified by flash column chromatography (silica, 0 to 25% methanol in dichloromethane) to give ((2S)-4-(aminomethyl)tetrahydrofuran-2-yl)((S)-1-(4-fluorophenyl)-3,4-dihydroisoquinolin-2(1H)-yl)methanone.

LCMS: 100%, RT=1.78 min., (M+H)⁺=355 (method K).

### Synthesis of tert-butyl (((3R,5S)-5-((S)-1-(4-fluorophenyl)-1,2,3,4-tetrahydroisoquinoline-2-carbonyl)tetrahydrofuran-3-yl)methyl)carbamate and tert-butyl (((3S,5S)-5-((S)-1-(4-fluorophenyl)-1,2,3,4-tetrahydroisoquinoline-2-carbonyl)tetrahydrofuran-3-yl)methyl)carbamate

Di-tert-butyl dicarbonate (93 mg, 0.426 mmol) was added to a solution of ((2S)-4-(aminomethyl)tetrahydrofuran-2-yl)((S)-1-(4-fluorophenyl)-3,4-dihydroisoquinolin-2(1H)-yl)methanone (134 mg, 0.378 mmol) in dichloromethane (3 mL). After 5.5 hours, the reaction mixture was concentrated to dryness under reduced pressure and the residue purified by flash column chromatography (silica, 10 to 50% ethyl acetate in heptane) and preparative chiral SFC (method AP) to give tert-butyl (((3R,5S)-5-((S)-1-(4-fluorophenyl)-1,2,3,4-tetrahydroisoquinoline-2-carbonyl)tetrahydrofuran-3-yl)methyl)carbamate as the first eluting isomer and tert-butyl (((3S,5S)-5-((S)-1-(4-fluorophenyl)-1,2,3,4-tetrahydroisoquinoline-2-carbonyl)tetrahydrofuran-3-yl)methyl)carbamate as the second eluting isomer. The absolute configuration of the tetrahydropyran-aminomethyl center was arbitrarily assigned.

First eluting isomer: LCMS: 97%, RT=2.13 min., (M+Na)⁺=477 (method A). SFC: d.e.=100%, RT=3.66 min., (M+Na)⁺=477 (method A1).

Second eluting isomer: LCMS: 97%, RT=2.12 min., (M+H)⁺=455 (method A). SFC: d.e.=97%, RT=3.98 min., (M+H)⁺=455 (method A1).

### ((2S,4S)-4-(aminomethyl)tetrahydrofuran-2-yl)((S)-1-(4-fluorophenyl)-3,4-dihydroisoquinolin-2(1H)-yl)methanone (Compound 5008)

Trifluoroacetic acid (0.1 mL) was added to a solution of tert-butyl (((3R,5R)-5-((S)-1-(4-fluorophenyl)-1,2,3,4-tetrahydroisoquinoline-2-carbonyl)tetrahydrofuran-3-yl)methyl)carbamate (22.3 mg, 0.049 mmol) in dichloromethane (2.0 mL). After stirring at room temperature for 4.5 hours, the reaction mixture was evaporated under reduced pressure, desalted (SCX-2 (1 g) ion exchange chromatography), and lyophilized from a mixture of acetonitrile and water (1:1). ((2R,4R)-4-(aminomethyl)tetrahydrofuran-2-yl)((S)-1-(4-fluorophenyl)-3,4-dihydroisoquinolin-2(1H)-yl)methanone. The absolute configuration of the tetrahydropyran-aminomethyl center was arbitrarily assigned.

LCMS: 99%, RT=2.54 min., (M+H)⁺=355 (method AK).

### ((2S,4R)-4-(aminomethyl)tetrahydrofuran-2-yl)((S)-1-(4-fluorophenyl)-3,4-dihydroisoquinolin-2(1H)-yl)methanone (Compound 5009)

Starting from tert-butyl (((3R,5S)-5-((S)-1-(4-fluorophenyl)-1,2,3,4-tetrahydroisoquinoline-2-carbonyl)tetrahydrofuran-3-yl)methyl)carbamate (18.6 mg, 0.041 mmol), ((2S,4R)-4-(aminomethyl)tetrahydrofuran-2-yl)((S)-1-(4-fluorophenyl)-3,4-dihydroisoquinolin-2(1H)-yl)methanone (Compound 5009) was prepared as described for Compound 5008, desalted (SCX-2 (1 g) ion exchange chromatography), and lyophilized from a mixture of acetonitrile and water (1:1). The absolute configuration of the tetrahydropyran-aminomethyl center was arbitrarily assigned.

LCMS: 99%, RT=2.54 min., (M+H)⁺=355 (method AK).

### Example 6

### Synthesis of ((2S,4R)-4-aminotetrahydrofuran-2-yl)((S)-1-(4-fluorophenyl)-3,4-dihydroisoquinolin-2(1)-yl)methanone (Compound 5010) and ((2S,4S)-4-aminotetrahydrofuran-2-yl)((S)-1-(4-fluorophenyl)-3,4-dihydroisoquinolin-2(1H)-yl)methanone (Compound 5011)

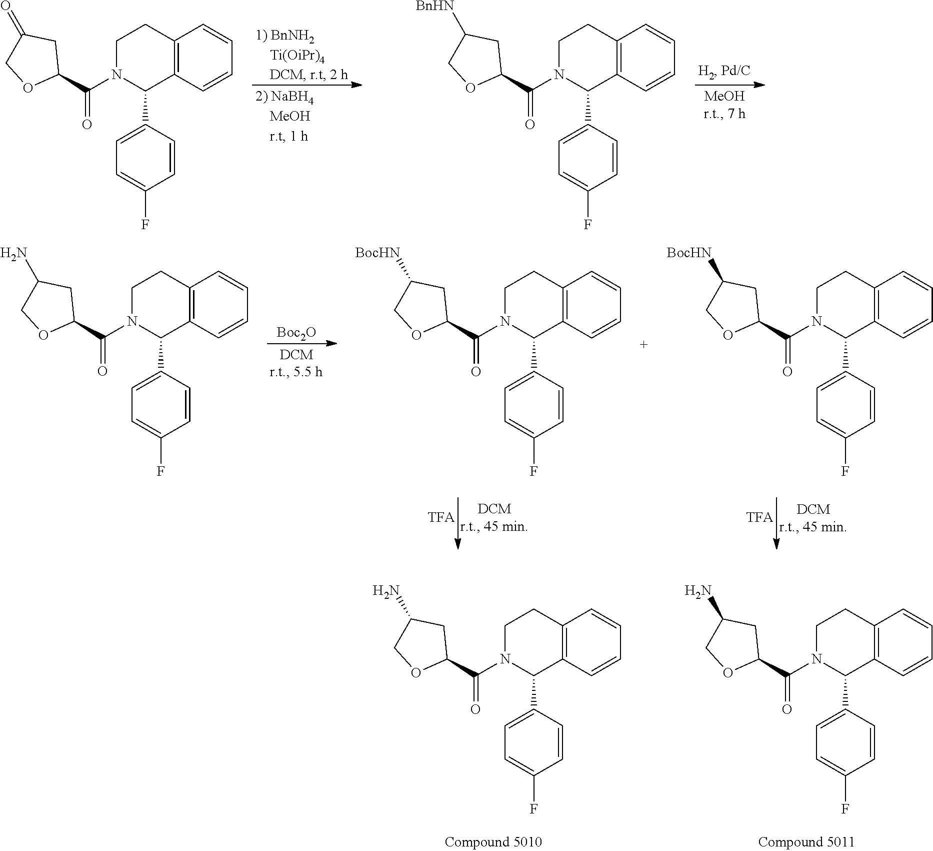

### Synthesis of ((2S)-4-(benzylamino)tetrahydrofuran-2-yl)((S)-1-(4-fluorophenyl)-3,4-dihydroisoquinolin-2(1H)-yl)methanone

Starting from (S)-5-((S)-1-(4-fluorophenyl)-1,2,3,4-tetrahydroisoquinoline-2-carbonyl)dihydrofuran-3(2H)-one (see Compound 5004, 290 mg, 0.855 mmol), ((2S)-4-(benzylamino)tetrahydrofuran-2-yl)((S)-1-(4-fluorophenyl)-3,4-dihydroisoquinolin-2(1H)-yl)methanone (368 mg) was prepared as described for ((2S)-4-((benzylamino)methyl)tetrahydrofuran-2-yl)((S)-1-(4-fluorophenyl)-3,4-dihydroisoquinolin-2(1H)-yl)methanone (see Compound 5008). Used as such.

LCMS: 63%, RT=2.22 min., (M+H)⁺=431 (method K).

### Synthesis of ((2S)-4-(benzylamino)tetrahydrofuran-2-yl)((S)-1-(4-fluorophenyl)-3,4-dihydroisoquinolin-2(1H)-yl)methanone

Starting from ((2S)-4-(benzylamino)tetrahydrofuran-2-yl)((S)-1-(4-fluorophenyl)-3,4-dihydroisoquinolin-2(1H)-yl)methanone (368 mg, 0.855 mmol), ((2S)-4-(benzylamino)tetrahydrofuran-2-yl)((S)-1-(4-fluorophenyl)-3,4-dihydroisoquinolin-2(1H)-yl)methanone was prepared as described for ((2S)-4-(aminomethyl)tetrahydrofuran-2-yl)((S)-1-(4-fluorophenyl)-3,4-dihydroisoquinolin-2(1H)-yl)methanone (see Compound 5008). and used without purification.

LCMS: 81%, RT=1.79 min., (M+H)⁺=341 (method K).

### Synthesis of tert-butyl ((3R,5S)-5-((S)-1-(4-fluorophenyl)-1,2,3,4-tetrahydroisoquinoline-2-carbonyl)tetrahydrofuran-3-yl)carbamate and tert-butyl ((3S,5S)-5-((S)-1-(4-fluorophenyl)-1,2,3,4-tetrahydroisoquinoline-2-carbonyl)tetrahydrofuran-3-yl)carbamate

Di-tert-butyl dicarbonate (131 mg, 0.600 mmol) was added to a suspension of ((2S)-4-(aminomethyl)tetrahydrofuran-2-yl)((S)-1-(4-fluorophenyl)-3,4-dihydroisoquinolin-2(1H)-yl)methanone (178 mg, 0.523 mmol) in dichloromethane (5 mL). After 5.5 hours, the reaction mixture was diluted with dichloromethane (10 mL) and saturated aqueous NaHCO₃ (2 mL). The layers were separated using a phase-separator and the organic filtrate was evaporated under reduced pressure. The residue was purified by flash column chromatography (silica, 10 to 50% ethyl acetate in heptane) and preparative chiral SFC (method S) to give tert-butyl ((3S,5S)-5-((S)-1-(4-fluorophenyl)-1,2,3,4-tetrahydroisoquinoline-2-carbonyl)tetrahydrofuran-3-yl)carbamate as the first eluting isomer and tert-butyl ((3R,5S)-5-((S)-1-(4-fluorophenyl)-1,2,3,4-tetrahydroisoquinoline-2-carbonyl)tetrahydrofuran-3-yl)carbamate as the second eluting isomer. The absolute configuration of the tetrahydropyran-amine center was arbitrarily assigned.

First eluting isomer: LCMS: 98%, RT=2.17 min., (M+Na)⁺=463 (method A). SFC: d.e.=100%, RT=2.64 min., (M+Na)⁺=463 (method F).

Second eluting isomer: LCMS: 98%, RT=2.12 min., (M+H)⁺=441 (method A). SFC: d.e.=98%, RT=3.14 min., (M+H)⁺=441 (method F).

### Synthesis of ((2S,4R)-4-aminotetrahydrofuran-2-yl)((S)-1-(4-fluorophenyl)-3,4-dihydroisoquinolin-2(1H)-yl)methanone (Compound 5010)

Starting from tert-butyl ((3R,5S)-5-((S)-1-(4-fluorophenyl)-1,2,3,4-tetrahydroisoquinoline-2-carbonyl)tetrahydrofuran-3-yl)carbamate (6.4 mg, 0.0145 mmol), ((2S,4R)-4-aminotetrahydrofuran-2-yl)((S)-1-(4-fluorophenyl)-3,4-dihydroisoquinolin-2(1H)-yl)methanone (Compound 5010) was prepared as described for Compound 5008, desalted (SCX-2 (1 g) ion exchange chromatography), and lyophilized from a mixture of acetonitrile and water (1:1). The absolute configuration of the tetrahydropyran-amine center was arbitrarily assigned.

LCMS: 98%, RT=2.50 min., (M+H)⁺=341 (method AK).

### Synthesis of ((2S,4S)-4-aminotetrahydrofuran-2-yl)((S)-1-(4-fluorophenyl)-3,4-dihydroisoquinolin-2(1H)-yl)methanone (Compound 5011)

Starting from tert-butyl ((3S,5S)-5-((S)-1-(4-fluorophenyl)-1,2,3,4-tetrahydroisoquinoline-2-carbonyl)tetrahydrofuran-3-yl)carbamate (55.7 mg, 0.126 mmol), ((2S,4S)-4-aminotetrahydrofuran-2-yl)((S)-1-(4-fluorophenyl)-3,4-dihydroisoquinolin-2(1H)-yl)methanone (Compound 5011) was prepared as described for Compound 5008, desalted (SCX-2 (1 g) ion exchange chromatography), and lyophilized from a mixture of acetonitrile and water (1:1). The absolute configuration of the tetrahydropyran-amine center was arbitrarily assigned.

LCMS: 100%, RT=2.54 min., (M+H)⁺=341 (method AK).

### Example 7

### Synthesis of ((2R,4R)-4-aminotetrahydrofuran-2-yl)((S)-1-(4-fluorophenyl)-3,4-dihydroisoquinolin-2(1H)-yl)methanone (Compound 5012) and ((2R,4S)-4-aminotetrahydrofuran-2-yl)((S)-1-(4-fluorophenyl)-3,4-dihydroisoquinolin-2(1H)-yl)methanone (Compound 5013)

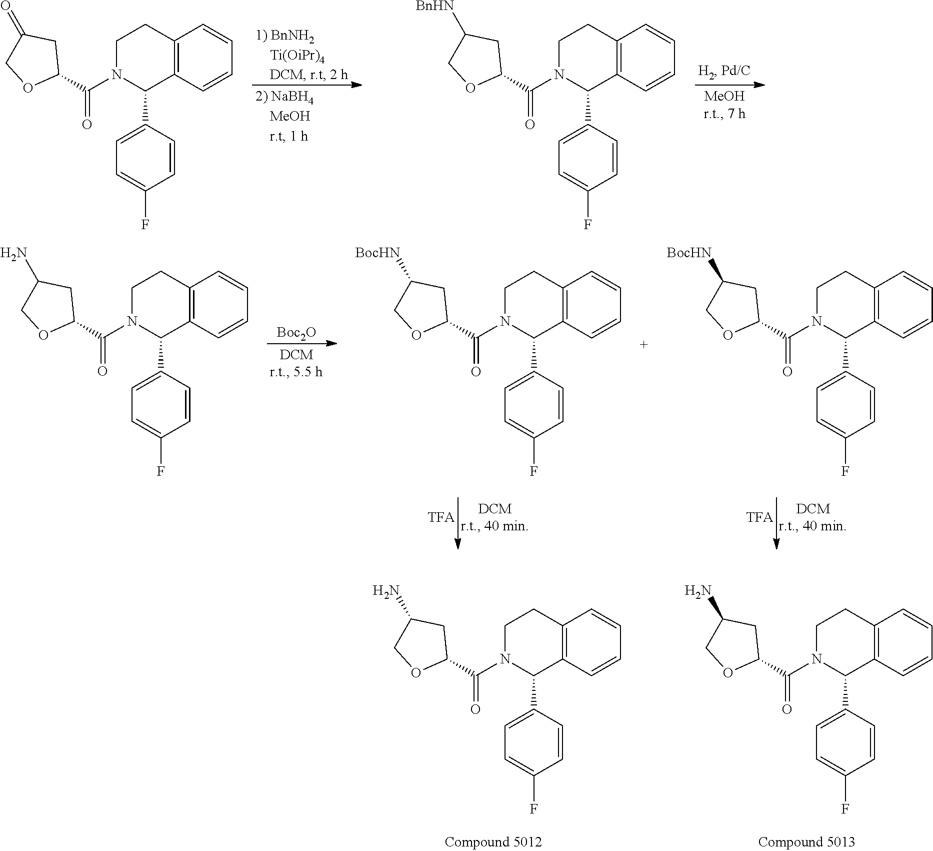

### Synthesis of ((2R)-4-(benzylamino)tetrahydrofuran-2-yl)((S)-1-(4-fluorophenyl)-3,4-dihydroisoquinolin-2(1H)-yl)methanone

Starting from (R)-5-((S)-1-(4-fluorophenyl)-1,2,3,4-tetrahydroisoquinoline-2-carbonyl)dihydrofuran-3(2H)-one (see Compound 5005, 290 mg, 0.855 mmol), ((2R)-4-(benzylamino)tetrahydrofuran-2-yl)((S)-1-(4-fluorophenyl)-3,4-dihydroisoquinolin-2(1H)-yl)methanone was prepared as described for ((2S)-4-((benzylamino)methyl)tetrahydrofuran-2-yl)((S)-1-(4-fluorophenyl)-3,4-dihydroisoquinolin-2(1H)-yl)methanone (see Compound 5008) and used directly in the next step.

LCMS: 64%, RT=2.24 min., (M+H)⁺=431 (method K).

### Synthesis of ((2R)-4-aminotetrahydrofuran-2-yl)((S)-1-(4-fluorophenyl)-3,4-dihydroisoquinolin-2(1H)-yl)methanone

Starting from ((2R)-4-(benzylamino)tetrahydrofuran-2-yl)((S)-1-(4-fluorophenyl)-3,4-dihydroisoquinolin-2(1H)-yl)methanone (368 mg, 0.855 mmol), ((2R)-4-(benzylamino)tetrahydrofuran-2-yl)((S)-1-(4-fluorophenyl)-3,4-dihydroisoquinolin-2(1H)-yl)methanone was prepared as described for ((2S)-4-(aminomethyl)tetrahydrofuran-2-yl)((S)-1-(4-fluorophenyl)-3,4-dihydroisoquinolin-2(1H)-yl)methanone (see Compound 5008) and used directly in the next step.

LCMS: 83%, RT=1.80 min., (M+H)⁺=341 (method K).

### Synthesis of tert-butyl ((3S,5R)-5-((S)-1-(4-fluorophenyl)-1,2,3,4-tetrahydroisoquinoline-2-carbonyl)tetrahydrofuran-3-yl)carbamate and tert-butyl ((3R,5R)-5-((S)-1-(4-fluorophenyl)-1,2,3,4-tetrahydroisoquinoline-2-carbonyl)tetrahydrofuran-3-yl)carbamate

Di-tert-butyl dicarbonate (194 mg, 0.889 mmol) was added to a suspension of ((2R)-4-(aminomethyl)tetrahydrofuran-2-yl)((S)-1-(4-fluorophenyl)-3,4-dihydroisoquinolin-2(1H)-yl)methanone (270 mg, 0.793 mmol) in dichloromethane (5 mL). After 5.5 hours, the reaction mixture was diluted with dichloromethane (10 mL) and saturated aqueous NaHCO₃ (2 mL). The layers were separated using a phase-separator and the organic filtrate was evaporated under reduced pressure. The residue was purified by flash column chromatography (silica, 10 to 50% ethyl acetate in heptane) and preparative chiral SFC (method AN) to give tert-butyl ((3S,5R)-5-((S)-1-(4-fluorophenyl)-1,2,3,4-tetrahydroisoquinoline-2-carbonyl)tetrahydrofuran-3-yl)carbamate as the first eluting isomer and tert-butyl ((3R,5R)-5-((S)-1-(4-fluorophenyl)-1,2,3,4-tetrahydroisoquinoline-2-carbonyl)tetrahydrofuran-3-yl)carbamate as the second eluting isomer. The absolute configuration of the tetrahydropyran-amine center was arbitrarily assigned.

First eluting isomer: LCMS: 91%, RT=2.17 min., (M+Na)⁺=463 (method A). SFC: d.e.=100%, RT=2.66 min., (M+a)⁺=463 (method AD).

Second eluting isomer: LCMS: 98%, RT=2.12 min., (M+H)⁺=441 (method A). SFC: d.e.=96.5%, RT=3.30 min., (M+H)⁺=441 (method AD).

### Synthesis of ((2R,4R)-4-aminotetrahydrofuran-2-yl)((S)-1-(4-fluorophenyl)-3,4-dihydroisoquinolin-2(1H)-yl)methanone (Compound 5012)

Starting from tert-butyl ((3R,5R)-5-((S)-1-(4-fluorophenyl)-1,2,3,4-tetrahydroisoquinoline-2-carbonyl)tetrahydrofuran-3-yl)carbamate (15.0 mg, 0.0341 mmol), ((2R,4R)-4-aminotetrahydrofuran-2-yl)((S)-1-(4-fluorophenyl)-3,4-dihydroisoquinolin-2(1H)-yl)methanone (Compound 5012) was prepared as described for Compound 5008, desalted (SCX-2 (1 g) ion exchange chromatography), and lyophilized from a mixture of acetonitrile and water (1:1). The absolute configuration of the tetrahydropyran-amine center was arbitrarily assigned.

LCMS: 97%, RT=2.50 min., (M+H)⁺=341 (method AK).

### Synthesis of ((2R,4S)-4-aminotetrahydrofuran-2-yl)((S)-1-(4-fluorophenyl)-3,4-dihydroisoquinolin-2(1H)-yl)methanone (Compound 5013)

Starting from tert-butyl ((3S,5R)-5-((S)-1-(4-fluorophenyl)-1,2,3,4-tetrahydroisoquinoline-2-carbonyl)tetrahydrofuran-3-yl)carbamate (91.0 mg, 0.207 mmol), ((2R,4S)-4-aminotetrahydrofuran-2-yl)((S)-1-(4-fluorophenyl)-3,4-dihydroisoquinolin-2(1H)-yl)methanone (Compound 5013) was prepared as described for Compound 5008, desalted (SCX-2 (1 g) ion exchange chromatography), and lyophilized from a mixture of acetonitrile and water (1:1). The absolute configuration of the tetrahydropyran-amine center was arbitrarily assigned.

LCMS: 100%, RT=2.55 min., (M+H)⁺=341 (method AK).

### Example 8

### Synthesis of ((2R)-4-(aminomethyl)tetrahydrofuran-2-yl)((S)-1-(4-fluorophenyl)-3,4-dihydroisoquinolin-2(1H)-yl)methanone (Compound 5014)

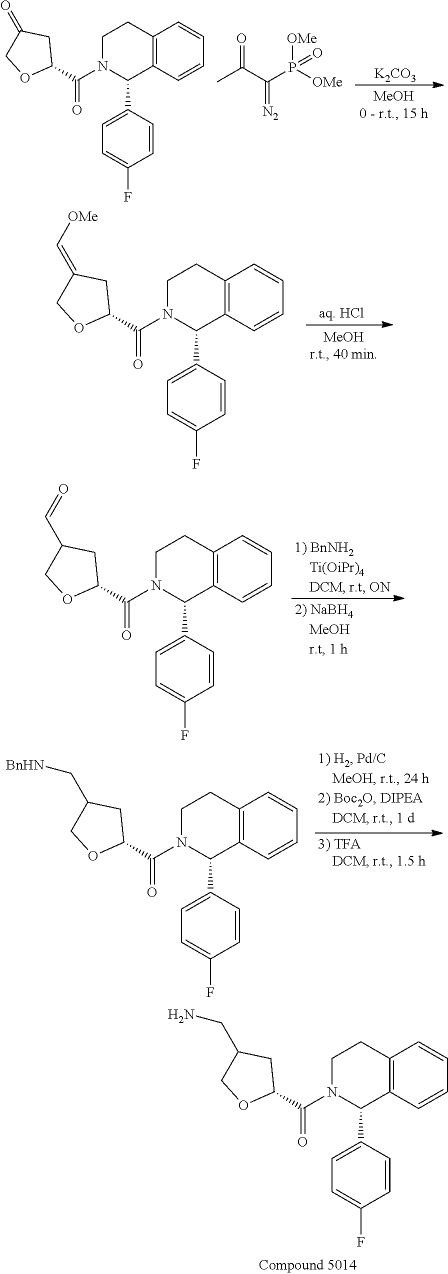

### Synthesis of ((S)-1-(4-fluorophenyl)-3,4-dihydroisoquinolin-2(1H)-yl)((R)-4-(methoxymethylene)tetrahydrofuran-2-yl)methanone

Starting from dimethyl (1-diazo-2-oxopropyl)phosphonate (270 mg, 1.41 mmol) and (R)-5-((S)-1-(4-fluorophenyl)-1,2,3,4-tetrahydroisoquinoline-2-carbonyl)dihydrofuran-3(2H)-one (see Compound 5005, 40 mg, 0.12 mmol), ((S)-1-(4-fluorophenyl)-3,4-dihydroisoquinolin-2(1H)-yl)((R)-4-(methoxymethylene)tetrahydrofuran-2-yl)methanone was prepared as described for ((S)-1-(4-fluorophenyl)-3,4-dihydroisoquinolin-2(1H)-yl)((S)-4-(methoxymethylene)tetrahydrofuran-2-yl)methanone (see Compound 5008).

LCMS: E/Z mixture, RT=2.20 and 2.22 min., (M+H)⁺=368 (method K).

### Synthesis of (5R)-5-((S)-1-(4-fluorophenyl)-1,2,3,4-tetrahydroisoquinoline-2-carbonyl)tetrahydrofuran-3-carbaldehyde

Starting from ((S)-1-(4-fluorophenyl)-3,4-dihydroisoquinolin-2(1H)-yl)((R)-4-(methoxymethylene)tetrahydrofuran-2-yl)methanone (40 mg, 0.11 mmol), (5R)-5-((S)-1-(4-fluorophenyl)-1,2,3,4-tetrahydroisoquinoline-2-carbonyl)tetrahydrofuran-3-carbaldehyde was prepared as described for (5S)-5-((S)-1-(4-fluorophenyl)-1,2,3,4-tetrahydroisoquinoline-2-carbonyl)tetrahydrofuran-3-carbaldehyde (see Compound 5008). Directly used in the next step.

LCMS: RT=2.02 min, (M+H)⁺=354 (method K).

### Synthesis of ((2R)-4-((benzylamino)methyl)tetrahydrofuran-2-yl)((S)-1-(4-fluorophenyl)-3,4-dihydroisoquinolin-2(1H)-yl)methanone product

Starting from crude (5R)-5-((S)-1-(4-fluorophenyl)-1,2,3,4-tetrahydroisoquinoline-2-carbonyl)tetrahydrofuran-3-carbaldehyde (0.11 mmol), ((2R)-4-((benzylamino)methyl)tetrahydrofuran-2-yl)((S)-1-(4-fluorophenyl)-3,4-dihydroisoquinolin-2(1H)-yl)methanone product was prepared as described for ((2S)-4-((benzylamino)methyl)tetrahydrofuran-2-yl)((S)-1-(4-fluorophenyl)-3,4-dihydroisoquinolin-2(1H)-yl)methanone (see Compound 5008). Directly used in the next step.

LCMS: RT=2.17 min., (M+H)⁺=445 (method K).

### Synthesis of ((2R)-4-(aminomethyl)tetrahydrofuran-2-yl)((S)-1-(4-fluorophenyl)-3,4-dihydroisoquinolin-2(1H)-yl)methanone

Starting from ((2R)-4-((benzylamino)methyl)tetrahydrofuran-2-yl)((S)-1-(4-fluorophenyl)-3,4-dihydroisoquinolin-2(1H)-yl)methanone (50 mg, 0.11 mmol), ((2R)-4-(aminomethyl)tetrahydrofuran-2-yl)((S)-1-(4-fluorophenyl)-3,4-dihydroisoquinolin-2(1H)-yl)methanone (Compound 5014) was prepared as described for ((2S)-4-(aminomethyl)tetrahydrofuran-2-yl)((S)-1-(4-fluorophenyl)-3,4-dihydroisoquinolin-2(1H)-yl)methanone (see Compound 5008) and purified as its Boc-protected derivative (di-tert-butyl dicarbonate (18.3 mg, 0.084 mmol), N,N-diisopropylethylamine (0.080 mL, 0.458 mmol) in dichloromethane (3 mL), 1 day; flash column chromatography (silica, 10 to 70% ethyl acetate in heptane)) and deprotected (trifluoroacetic acid (0.5 mL), dichloromethane (2 mL), 1.5 hours; SCX-2 (1 g)) then lyophilized lyophilization from acetonitrile and water (1:1).

LCMS: 95%, RT=1.02 min., (M+H)⁺=355 (method P).

### Example 9

### Synthesis of ((2S,5R)-5-aminotetrahydro-2H-pyran-2-yl)((S)-1-(4-fluorophenyl)-3,4-dihydroisoquinolin-2(1H)-yl)methanone (Compound 5015)

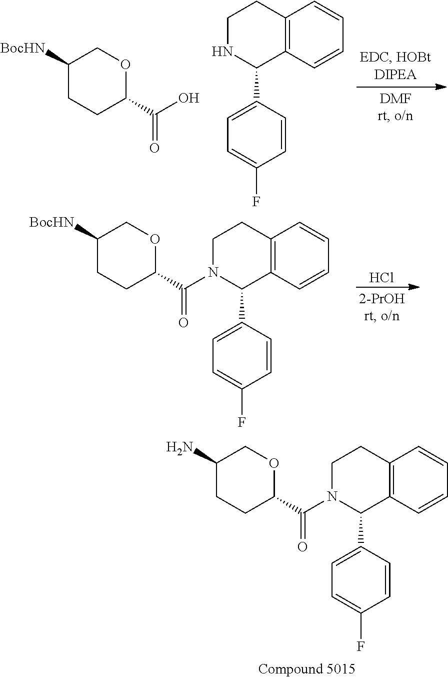

### Synthesis of tert-butyl ((3R,6S)-6-((S)-1-(4-fluorophenyl)-1,2,3,4-tetrahydroisoquinoline-2-carbonyl)tetrahydro-2H-pyran-3-yl)carbamate

Starting from (2S,5R)-5-{[(tert-butoxy)carbonyl]amino}oxane-2-carboxylic acid (1.00 g, 4.08 mmol) and (S)-1-(4-fluorophenyl)-1,2,3,4-tetrahydroisoquinoline (0.927 g, 4.08 mmol), tert-butyl ((3R,6S)-6-((S)-1-(4-fluorophenyl)-1,2,3,4-tetrahydroisoquinoline-2-carbonyl)tetrahydro-2H-pyran-3-yl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)carbamate (see Compound 5042) and purified by flash column chromatography (silica, 0 to 50% ethyl acetate in heptane).

LCMS: 99%, RT=2.14 min., (M+H)⁺=455 (method A).

### Synthesis of ((2S,5R)-5-aminotetrahydro-2H-pyran-2-yl)((S)-1-(4-fluorophenyl)-3,4-dihydroisoquinolin-2(1H)-yl)methanone (Compound 5015)

HCl (5-6 M in 2-propanol, 15 mL, 75 mmol) was added to a solution of tert-butyl ((3R,6S)-6-((S)-1-(4-fluorophenyl)-1,2,3,4-tetrahydroisoquinoline-2-carbonyl)tetrahydro-2H-pyran-3-yl)carbamate (1.71 g, 3.76 mmol) in 2-propanol (30 mL) and stirred overnight. The reaction mixture was concentrated to dryness under reduced pressure. The residue was diluted with water (40 mL) and aqueous HCl (1 M, 4.0 mL (pH<3)) and the mixture extracted with ethyl acetate (20 mL). The aqueous phase was basified by addition of saturated aqueous K₂CO₃ (20 mL) and extracted with ethyl acetate (50 mL). The latter organic layer was dried over Na₂SO₄ and evaporated under reduced pressure to give ((2S,5R)-5-aminotetrahydro-2H-pyran-2-yl)((S)-1-(4-fluorophenyl)-3,4-dihydroisoquinolin-2(1H)-yl)methanone (Compound 5015) after lyophilization from a mixture of acetonitrile and water (1:1, 30 mL).

LCMS: 99%, RT=1.03 min., (M+H)⁺=355 (method P).

### Example 10

### Synthesis of Compound 5016 and Compound 5017

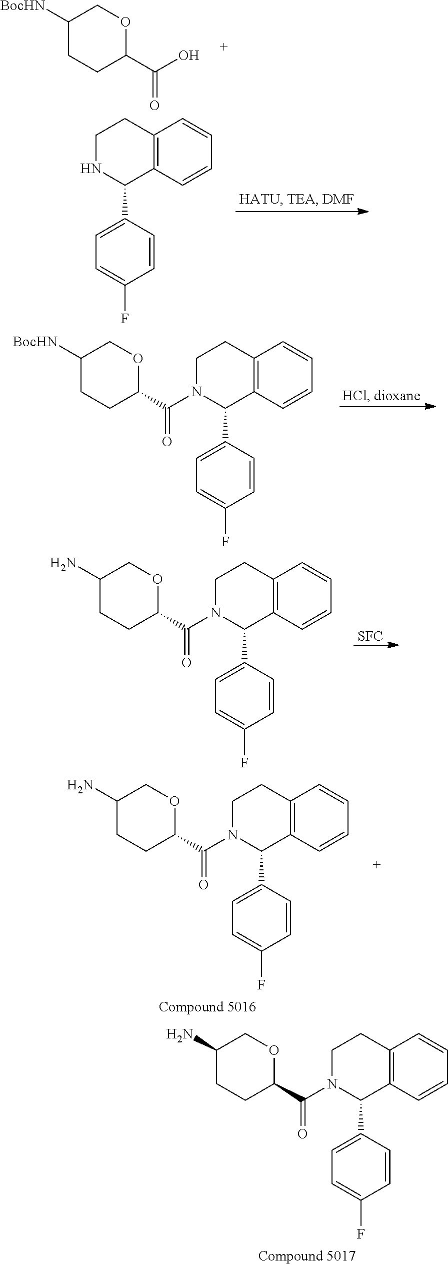

Step 1: To a solution of 5-(tert-butoxycarbonylamino)-tetrahydro-2H-pyran-2-carboxylic acid (200 mg, 0.8 mmol) in DMF (4 mL) were added (S)-1-(4-fluorophenyl)-1,2,3,4-tetrahydroisoquinoline (185 mg, 0.8 mmol), HATU (372 mg, 1.0 mmol) and Et₃N (0.2 mL, 1.6 mmol) at 0° C. The resulting reaction mixture was stirred at room temperature for 2 h and then diluted with ethyl acetate (20 mL) and water (30 mL). The aqueous layer was extracted with ethyl acetate (3×30 mL) and the combined organic phase washed with brine (30 mL), dried over Na₂SO₄, filtered and concentrated. The residue was purified by column chromatography to give tert-butyl 6-((S)-1-(4-fluorophenyl)-1,2,3,4-tetrahydroisoquinoline-2-carbonyl)-tetrahydro-2H-pyran-3-ylcarbamate.

LCMS: (M+H)⁺=455; Retention time=1.933 min. LCMS CP Method A

Step 2: To a solution of tert-butyl 6-((S)-1-(4-fluorophenyl)-1,2,3,4-tetrahydroisoquinoline-2-carbonyl)-tetrahydro-2H-pyran-3-ylcarbamate (300 mg, 0.7 mmol) in dioxane (2 mL) was added HCl in dioxane (4N, 2 mL) at 0° C. The reaction mixture was stirred at room temperature for 2 h and then concentrated under reduced pressure. The residue was purified by Prep-HPLC to give (5-amino-tetrahydro-2H-pyran-2-yl)((S)-1-(4-fluorophenyl)-3,4-dihydroisoquinolin-2(1H)-yl)methanone.

LCMS: (M+H)⁺=355; Retention time=1.381 min. LCMS CP Method E

The diastereomers were separated by chiral SFC eluting with CO₂/MeOH containing 0.2% MA over an EnantioPak® AD column (20*250 mm 10 μm) to give Compound 5016 (retention time=3.332 min), Compound 5017 (retention time=1.193 min). Stereochemical assignment of (S) at the 1 position of the tetrahydroisoquinoline is based on enantiomerically pure starting materials; the configuration at the tetrahydropyran chiral center is assigned based on chromatographic elution order as compared to related analogues of known configuration.

Compound 5016: LCMS: (M+H)⁺=355; purity=100% (214 nm); retention time=1.490 min. LCMS CP Method F

Chiral SFC: CO₂/MeOH containing 0.2% MA (65%:35%) over a CHIRALPAK® IG column (4.6*100 mm 5 μm), retention time=3.086 min, 100% ee.

Compound 5017: LCMS: (M+H)⁺=355; purity=97.36% (214 nm); retention time=1.489 min. LCMS CP Method F

### Example 11

### Synthesis of Compound 5018 and Compound 5019

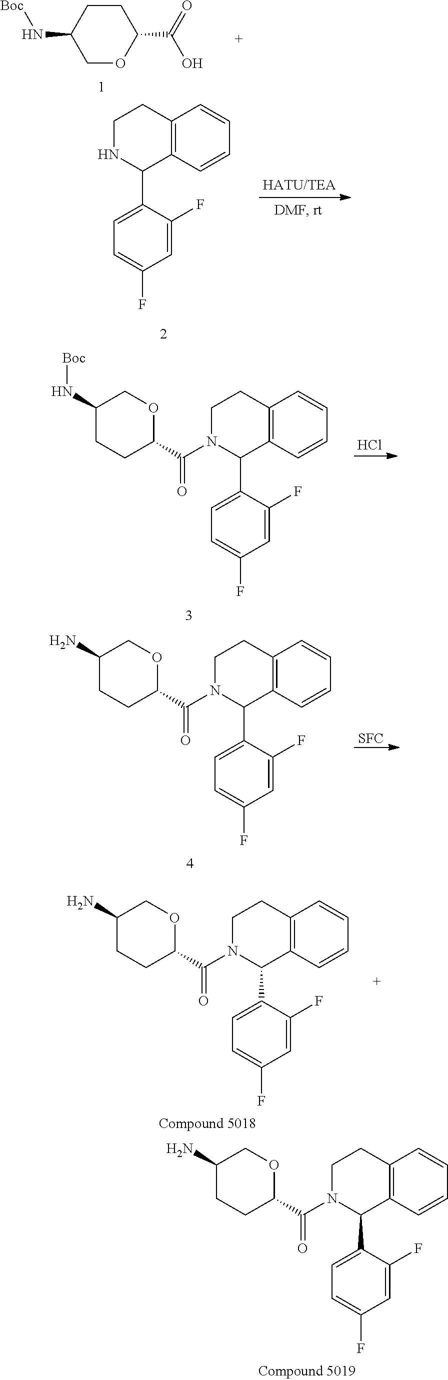

Step 1: To a solution of 1-(2,4-difluorophenyl)-1,2,3,4-tetrahydroisoquinoline (300 mg, 1.2 mmol) in DMF (5 mL) were added (2R,5S)-5-((tert-butoxycarbonyl)amino)tetrahydro-2H-pyran-2-carboxylic acid (223 mg, 0.99 mmol), HATU (480 mg, 1.26 mmol) and TEA (0.3 mL, 2 mmol) at 0° C. The resulting reaction mixture was stirred at room temperature for 2 h, diluted with EA (10 mL) and washed with saturated NH₄Cl (2×10 mL) followed by brine (2×10 mL). The organic phase was dried over Na₂SO₄ and concentrated in vacuo. The residue was purified by Prep-HPLC to give tert-butyl ((3S,6R)-6-(1-(2,4-difluorophenyl)-1,2,3,4-tetrahydroisoquinoline-2-carbonyl)tetrahydro-2H-pyran-3-yl)carbamate

LCMS: (M+H)⁺=473.1; purity=100% (214 nm); Retention time=1.68 min. Method C1

Step 2: To a round bottomed flask charged with a solution of HCl in Dioxane (10 mL, 4.0 M) was added tert-butyl ((3S,6R)-6-(1-(2,4-difluorophenyl)-1,2,3,4-tetrahydroisoquinoline-2-carbonyl)tetrahydro-2H-pyran-3-yl)carbamate (320 mg, 0.68 mmol) and the reaction mixture was stirred at room temperature for 1 h. The mixture was concentrated to give a residue which was purified by Prep-HPLC to give ((2R,5S)-5-aminotetrahydro-2H-pyran-2-yl)((R)-1-(2,4-difluorophenyl)-3,4-dihydroisoquinolin-2(1H)-yl)methanone.

LCMS: (M+H)⁺=373.1; purity=100% (214 nm); Retention time=1.42 min. Method C1

The diastereomers were separated by chiral SFC eluting with CO2/MeOH (0.2% Methanol Ammonia)=45/55 over a Daicel® IG column (20×250 mm, 10 μm) to give Compound 5018 and Compound 5019. The configuration of the stereocenters on the pyran ring is based on commercial starting material; the tetrahydroisoquinoline stereochemical configuration is arbitrarily assign based on the chromatographic elution order of related analogs.

Compound 5018: LCMS:(M+H)⁺=373.0 purity=100% (214 nm); Retention time=1.49 min. Method C1

Chiral SFC: CO₂/MeOH containing 0.2% Methanol Ammonia=60:40 over Daicel® IG column (20*250 mm, 10 um), retention time=2.698 min), 98.7% ee.

Compound 5019: LCMS: (M+H)⁺=373.0; purity=100% (214 nm); Retention time=1.43 min. Method C1

Chiral SFC: CO₂/MeOH containing 0.2% Methanol Ammonia=60:40 over Daicel® IG column (20*250 mm 10 μm), retention time=1.503 min), 100% ee.

### Example 12

### Synthesis of Compound 5020, Compound 5021, Compound 5022 and Compound 5023

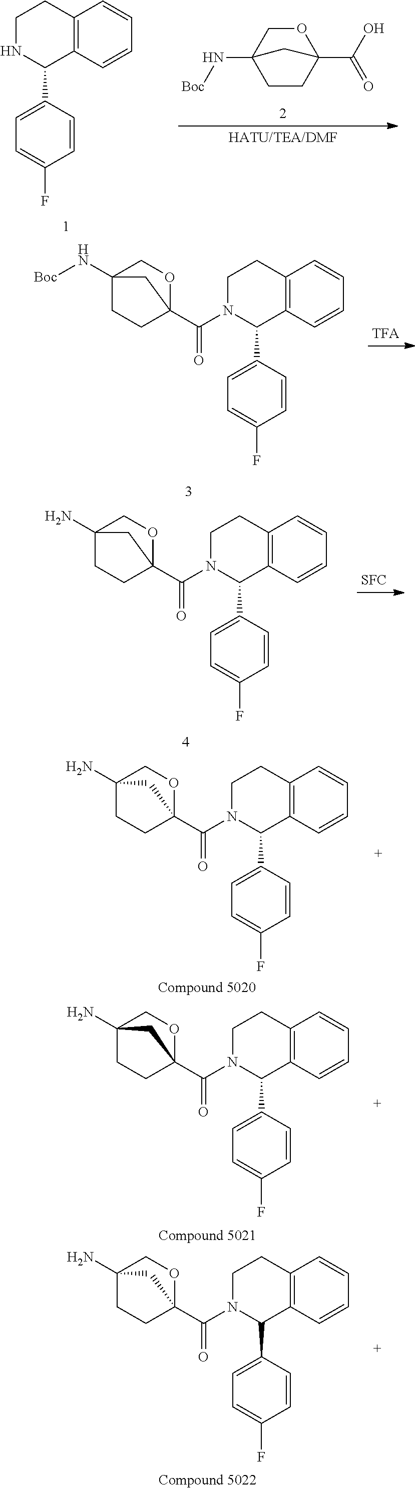

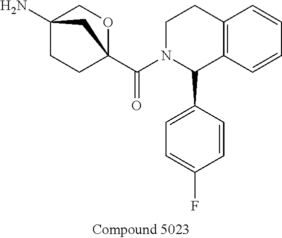

Step 1: To a solution of 4-((tert-butoxycarbonyl)amino)-2-oxabicyclo[2.2.1]heptane-1-carboxylic acid (150 mg, 0.58 mmol) and TEA (0.16 mL, 0.17 mmol) in DMF (2 mL) was added HATU (266 mg, 0.7 mmol) at room temperature. After stirring for 30 min at room temperature, (S)-1-(4-fluorophenyl)-1,2,3,4-tetrahydroisoquinoline (145 mg, 0.64 mmol) was added and the reaction mixture was stirred for 16 h at room temperature. The reaction mixture was filtered and the filtrate concentrated to give a residue which was purified by Prep-HPLC to give tert-butyl (1-((S)-1-(4-fluorophenyl)-1,2,3,4-tetrahydroisoquinoline-2-carbonyl)-2-oxabicyclo[2.2.1]heptan-4-yl)carbamate.

LCMS: (M+1)⁺=467.0; Retention time=1.78 min. LCMS CP Method C2

Step 2: To a solution of tert-butyl (1-((S)-1-(4-fluorophenyl)-1,2,3,4-tetrahydroisoquinoline-2-carbonyl)-2-oxabicyclo[2.2.1]heptan-4-yl)carbamate (62 mg, 0.13 mmol) in DCM (2 mL) was added TFA (0.5 mL) at 0° C. The reaction mixture was stirred at room temperature for 2 h. then concentrated and the residue redissolved in water. The mixture was basified with 1 N NaOH and extracted with DCM (3×20 mL). The combined organic layers were dried with anhydrous Na₂SO₄, filtered and concentrated to give a residue which was purified by Prep-HPLC to give (4-amino-2-oxabicyclo[2.2.1]heptan-1-yl)((S)-1-(4-fluorophenyl)-3,4-dihydroisoquinolin-2(1H)-yl)methanone.

LCMS: (M+H)⁺=367.0; Retention time=1.47 min. LCMS CP Method C2

The diastereomers (26 mg) were separated by chiral SFC eluting with C02/MEOH (0.2% Methanol Ammonia)=50/50 over a Daicel® IG column (20×250 mm, 10 μm) to give Compound 5020, Compound 5021, Compound 5022 and Compound 5023. Stereochemical assignment of (S) at the 1 position of the tetrahydroisoquinoline is assigned based on the starting material while stereochemical assignments on the bicyclic centers are arbitrary.

Compound 5020: LCMS: (M+H)⁺=367.0; Retention time=1.79 min. LCMS CP Method C

Chiral SFC: CO₂/IPA (60%:40%) containing 1% ammonia over CHIRALPAK® IG column (4.6×100 mm, 5 μm), retention time=1.462 min), 99.59% ee.

Compound 5021: LCMS: (M+H)⁺=367.2; Retention time=1.53 min. LCMS CP Method A1

Chiral SFC: CO₂/IPA (60%:40%) containing 1% ammonia over CHIRALPAK® IG column (4.6×100 mm, 5 μm), retention time=3.957 min), 80.32% ee.

Compound 5022: LCMS: (M+H)⁺=367.0; Retention time=1.76 min. LCMS CP Method C

Chiral SFC: CO₂/IPA (60%:40%) containing 1% ammonia over CHIRALPAK® IG column (4.6×100 mm, 5 μm), retention time=1.843 min), 95.5% ee.

Compound 5023: LCMS: (M+H)⁺=367.0; Retention time=1.78 min. LCMS CP Method C

Chiral SFC: CO₂/IPA (60%:40%) containing 1% ammonia over CHIRALPAK® IG column (4.6×100 mm, 5 μm), retention time=2.947 min), 96.64% ee.

### Example 13

### Synthesis of ((2S,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5024)

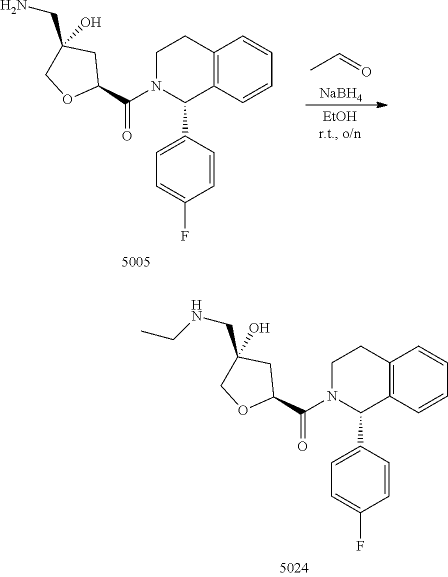

Synthesis of ((2S,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5024).

Acetaldehyde (10 wt % solution in ethanol, 73.0 μL, 0.130 mmol) was added to a solution of ((2S,4S)-4-(aminomethyl)-4-hydroxytetrahydrofuran-2-yl)((S)-1-(4-fluorophenyl)-3,4-dihydroisoquinolin-2(1H)-yl)methanone (Compound 5005, 32 mg, 0.086 mmol) was in ethanol (1.5 mL). The mixture was stirred for 1 hour, after which sodium borohydride (4.90 mg, 0.130 mmol) was added. After stirring overnight, water was added and the mixture filtered. The filtrate was evaporated under reduced pressure and the residue was purified by preparative SFC (method AT) and flash column chromatography (silica, 0 to 10% (3.5M ammonia in methanol) in dichloromethane) to give ((2S,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5024). The absolute configuration of the tetrahydropyran-2-carboxamide was determined by X-ray and the stereochemistry of tetrahydropyran-4-hydroxy-aminomethyl center was arbitrarily assigned.

LCMS: 96%, RT=2.56 min., (M+H)⁺=399 (method AK). SFC: RT=2.78 min.,

### Example 14

### Synthesis of Compound 5027 and Compound 5027

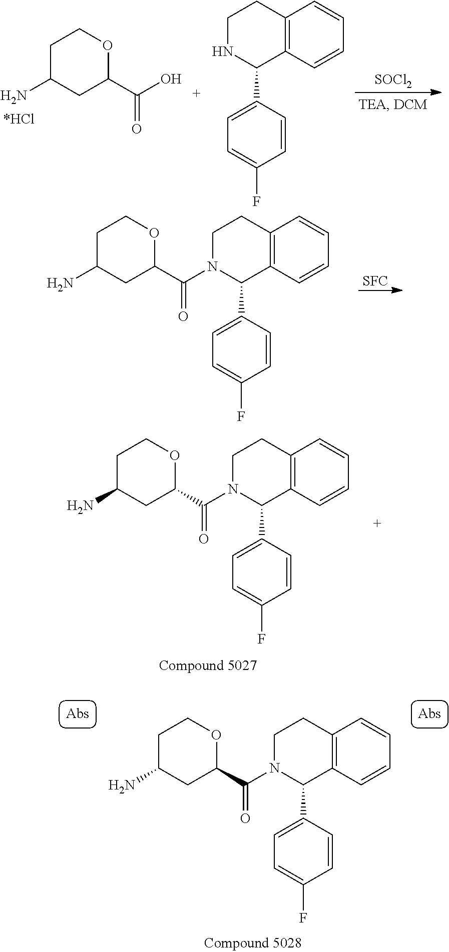

To a solution of 4-aminotetrahydro-2H-pyran-2-carboxylic acid hydrochloride (362 mg, 2 mmol) in DCM (50 mL) was added SOCl₂ (1 mL, excess) under nitrogen atmosphere. The resulting reaction mixture was stirred at room temperature for 1 h and then concentrated to give a white solid which was added to another solution of (S)-1-(4-fluorophenyl)-1,2,3,4-tetrahydroisoquinoline (454 mg, 2 mmol) and TEA (404 mg, 4 mmol) in DCM (10 mL). The resulting mixture was stirred for 3 h and then quenched with water (10 mL). The mixture was extracted with DCM (3×10 mL) and the combined organic phase was washed with brine (20 mL), dried over anhydrous Na₂SO₄, filtered and concentrated to give a residue. This was purified by Prep-HPLC to give (4-aminotetrahydro-2H-pyran-2-yl)((S)-1-(4-fluorophenyl)-3,4-dihydroisoquinolin-2(1H)-yl)methanone.

LCMS: (M+H)⁺=355.1, purity=100% (214 nm), Retention time=1.38 min. LCMS CP method C

The diastereomers were separated by chiral SFC eluting with CO₂/MEOH (0.2% Methanol Ammonia)=65/35 over an EnantioPak® OX-H column (20*250 mm 10 μm) to give Compound 5028 (retention time=2.32 min) and Compound 5027 (retention time=3.07 min). Stereochemical assignment of the tetrahydroisoquinoline is based on enantiomerically pure starting material and the tetrahydropyran stereochemistry was determined by X-Ray crystallography of related analogs.

Compound 5028: LCMS: ((M+H)⁺=355.1, purity=100% (214 nm), Retention time=1.38 min. LCMS CP method C

Chiral SFC: CO₂/MeOH containing 0.2% Methanol Ammonia=65:35 over CHIRALPAK® IG column (4.6*100 mm 5 μm), retention time=1.73 min, 100% ee.

Compound 5027: LCMS: ((M+H)⁺=355.1, purity=100% (214 nm), Retention time=1.38 min. LCMS CP method C

Chiral SFC: CO₂/MeOH containing 0.2% Methanol Ammonia=65:35 over CHIRALPAK® IG column (4.6*100 mm 5 μm), retention time=1.67 min, 100% ee.

### Example 15

### Synthesis of ((S)-1-(4-fluorophenyl)-3,4-dihydroisoquinolin-2(1H)-yl)((2S,5S)-5-((2-methoxyethyl)amino)tetrahydro-2H-pyran-2-yl)methanone (Compound 5029)

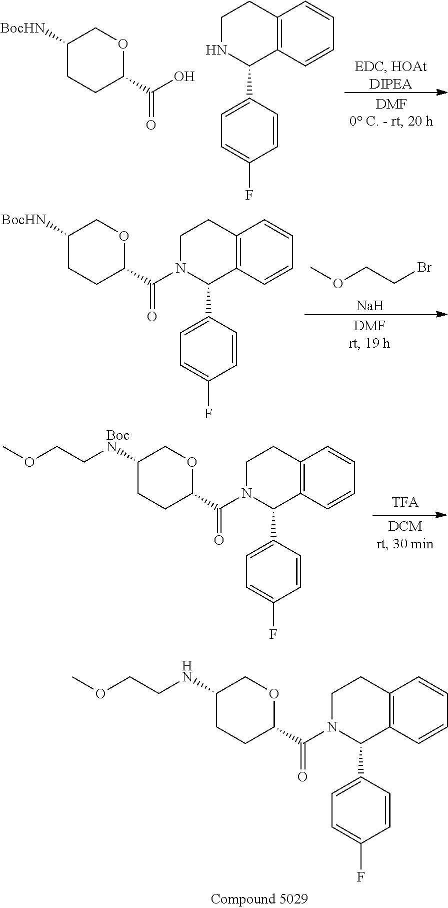

### Synthesis of tert-butyl ((3S,6S)-6-((S)-1-(4-fluorophenyl)-1,2,3,4-tetrahydroisoquinoline-2-carbonyl)tetrahydro-2H-pyran-3-yl)carbamate

Starting from (2S,5S)-5-((tert-butoxycarbonyl)amino)tetrahydro-2H-pyran-2-carboxylic acid (241 mg, 0.983 mmol) and (S)-1-(4-fluorophenyl)-1,2,3,4-tetrahydroisoquinoline (213 mg, 0.937 mmol), tert-butyl ((3S,6S)-6-((S)-1-(4-fluorophenyl)-1,2,3,4-tetrahydroisoquinoline-2-carbonyl)tetrahydro-2H-pyran-3-yl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)carbamate (see Compound 5042). White foam.

LCMS: 97%, RT=2.15 min., (M+Na)⁺=477 (method A).

### Synthesis of tert-butyl ((3S,6S)-6-((S)-1-(4-fluorophenyl)-1,2,3,4-tetrahydroisoquinoline-2-carbonyl)tetrahydro-2H-pyran-3-yl)(2-methoxyethyl)carbamate

Starting from tert-butyl ((3S,6S)-6-((S)-1-(4-fluorophenyl)-1,2,3,4-tetrahydroisoquinoline-2-carbonyl)tetrahydro-2H-pyran-3-yl)carbamate (76.6 mg, 0.169 mmol), tert-butyl ((3S,6S)-6-((S)-1-(4-fluorophenyl)-1,2,3,4-tetrahydroisoquinoline-2-carbonyl)tetrahydro-2H-pyran-3-yl)(2-methoxyethyl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate (see Compound 5030) and purified by acidic preparative MPLC (Linear Gradient: t=0 min 10% A; t=1 min 10% A; t=2 min 40% A; t=17 min 80% A; t=18 min 100%; t=23 min 100% A; detection: 220 nm).

LCMS: 100%, RT=2.27 min., (M+H)⁺=513 (method A).

### Synthesis of ((S)-1-(4-fluorophenyl)-3,4-dihydroisoquinolin-2(1H)-yl)((2S,5S)-5-((2-methoxyethyl)amino)tetrahydro-2H-pyran-2-yl)methanone (Compound 5029)

Starting from tert-butyl ((3S,6S)-6-((S)-1-(4-fluorophenyl)-1,2,3,4-tetrahydroisoquinoline-2-carbonyl)tetrahydro-2H-pyran-3-yl)(2-methoxyethyl)carbamate (44.8 mg, 0.087 mmol), ((S)-1-(4-fluorophenyl)-3,4-dihydroisoquinolin-2(1H)-yl)((2S,5S)-5-((2-methoxyethyl)amino)tetrahydro-2H-pyran-2-yl)methanone (Compound 5029) was prepared as described for ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030) without additional purification.

LCMS: 99%, RT=1.09 min., (M+H)⁺=413 (method P).

### Example 16

### Synthesis of ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030)

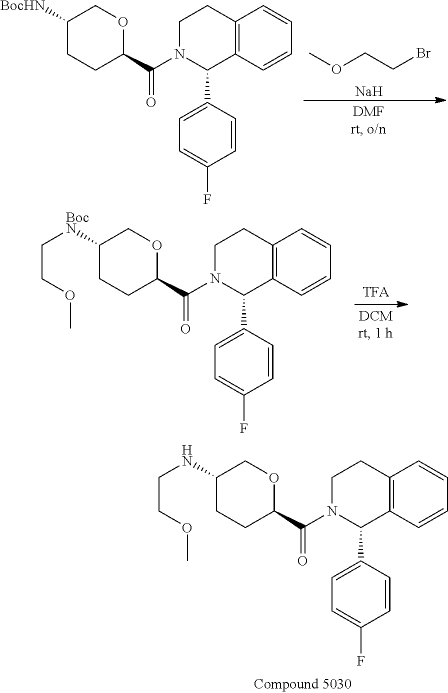

### Synthesis of tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate

Under nitrogen atmosphere, sodium hydride (60% dispersion in oil, 11.4 mg, 0.286 mmol) was added to a solution of tert-butyl ((3S,6R)-6-((S)-1-(4-fluorophenyl)-1,2,3,4-tetrahydroisoquinoline-2-carbonyl)tetrahydro-2H-pyran-3-yl)carbamate (See Compound 5042, 100 mg, 0.220 mmol) in N,N-dimethylformamide (dry, 1.5 mL). After 15 minutes, 2-bromoethyl methyl ether (38 μL, 0.396 mmol) was added and the stirring continued overnight. The reaction mixture was diluted with ethyl acetate (25 mL), washed with brine (4×10 mL), dried over Na₂SO₄, and evaporated under reduced pressure to give tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate which was used directly in the next step.

LCMS: 79%, RT=2.27 min., (M+H)⁺=513 (method A).

### Synthesis of ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030)

Trifluoroacetic acid (0.307 mL, 3.98 mmol) was added to a solution of tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate (102 mg (79 wt %)), 0.157 mmol) in dichloromethane (2.7 mL). After 1 hour, the reaction mixture was diluted with dichloromethane (15 mL) and washed with saturated aqueous NaHCO₃ (10 mL). The aqueous layer was extracted with dichloromethane (10 mL) and the combined organic layers were dried over Na₂SO₄ and evaporated under reduced pressure. The residue was dissolved in dimethyl sulfoxide (2 mL) and purified by acidic preparative MPLC (Linear Gradient: t=0 min 5% A; t=1 min 5% A; t=16.6 min 45% A; t=17.6 min 100%; t=22.6 min 100% A; detection: 220 nm). The product containing fractions were pooled, diluted with a mixture of brine, saturated aqueous NaHCO₃, and saturated aqueous Na₂CO₃ (1:1:1, 15 mL) and extracted with dichloromethane (3×20 mL). The combined organic phase was dried over Na₂SO₄ and evaporated under reduced pressure to give ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030) after lyophilization from a mixture of acetonitrile and water (3:2, 2 mL).

LCMS: 99%, RT=1.07 min., (M+H)⁺=413 (method P). SFC: RT=3.84 min.

### Example 17

### Synthesis of ((2S,4R)-4-((ethylamino)methyl)-4-hydroxytetrahydrofuran-2-yl)((S)-1-(4-fluorophenyl)-3,4-dihydroisoquinolin-2(1H)-yl)methanone (Compound 5031)

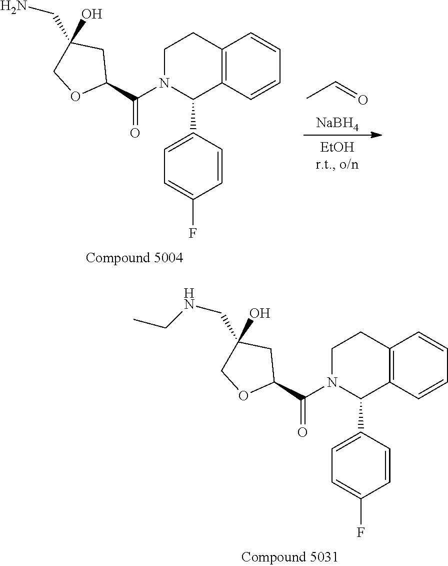

### Synthesis of ((2S,4R)-4-((ethylamino)methyl)-4-hydroxytetrahydrofuran-2-yl)((S)-1-(4-fluorophenyl)-3,4-dihydroisoquinolin-2(1H)-yl)methanone (Compound 5031)

Acetaldehyde (10 wt % solution in ethanol, 120 μL, 0.207 mmol) was added to a solution of ((2S,4R)-4-(aminomethyl)-4-hydroxytetrahydrofuran-2-yl)((S)-1-(4-fluorophenyl)-3,4-dihydroisoquinolin-2(1H)-yl)methanone (Compound 5004, 51 mg, 0.138 mmol) in ethanol (1.5 mL). The mixture was stirred for 1 hour, after which sodium borohydride (7.81 mg, 0.207 mmol) was added. After stirring overnight, water (few drops) and methanol (2 mL) were added and the mixture was filtered through a 0.45 μm nylon filter. The filtrate was evaporated under reduced pressure and the residue was purified by flash column chromatography (silica, 0 to 10% (7M ammonia in methanol) in dichloromethane) and acidic preparative MPLC (Linear Gradient: t=0 min 10% A; t=1 min 5% A; t=16 min 50% A; t=17 min 100%; t=22 min 100% A; detection: 220 nm) to give ((2S,4R)-4-((ethylamino)methyl)-4-hydroxytetrahydrofuran-2-yl)((S)-1-(4-fluorophenyl)-3,4-dihydroisoquinolin-2(1H)-yl)methanone (Compound 5031) after lyophilization from a mixture of acetonitrile and water (1:1, 4 mL). The absolute configuration of the tetrahydropyran-2-carboxamide was determined by X-ray and the stereochemistry of tetrahydropyran-4-hydroxy-aminomethyl center was arbitrarily assigned

LCMS: 98%, RT=1.03 min., (M+H)⁺=399 (method P).

### Example 18

### Synthesis of ((S)-1-(4-fluorophenyl)-3,4-dihydroisoquinolin-2(1H)-yl)((2S,5R)-5-((2-hydroxyethyl)amino)tetrahydro-2H-pyran-2-yl)methanone (Compound 5032)

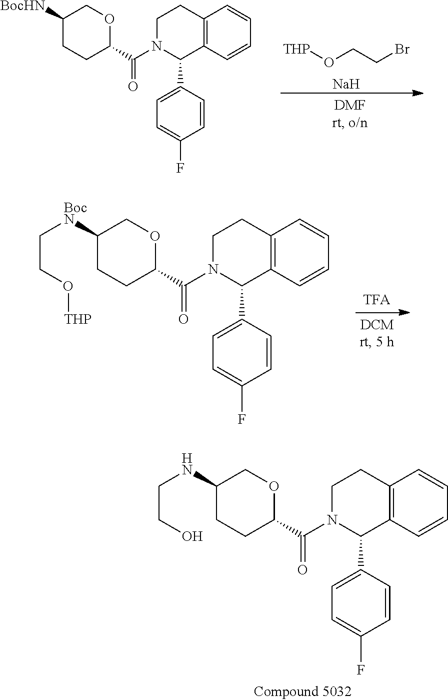

### Synthesis of tert-butyl ((3R,6S)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate

Starting from tert-butyl ((3R,6S)-6-((S)-1-(4-fluorophenyl)-1,2,3,4-tetrahydroisoquinoline-2-carbonyl)tetrahydro-2H-pyran-3-yl)carbamate (See Compound 5015, 100 mg, 0.220 mmol) and 2-(2-bromoethoxy)tetrahydro-2H-pyran (53 μL, 0.352 mmol), tert-butyl ((3R,6S)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate (see Compound 5030) and purified by basic preparative MPLC (Linear Gradient: t=0 min 10% A; t=1 min 10% A; t=2 min 40% A; t=17 min 80% A; t=18 min 100% A; t=23 min 100% A; detection: 220 nm).

LCMS: 98%, RT=2.26 min., (M-THP+H)⁺=499 (method A).

### Synthesis of ((S)-1-(4-fluorophenyl)-3,4-dihydroisoquinolin-2(1H)-yl)((2S,5R)-5-((2-hydroxyethyl)amino)tetrahydro-2H-pyran-2-yl)methanone (Compound 5032)

Starting from tert-butyl ((3R,6S)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate (70 mg, 0.120 mmol), ((S)-1-(4-fluorophenyl)-3,4-dihydroisoquinolin-2(1H)-yl)((2S,5R)-5-((2-hydroxyethyl)amino)tetrahydro-2H-pyran-2-yl)methanone (Compound 5032) was prepared as described for ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030). Two additional portions of trifluoroacetic acid were added after 2 and 4 hours, respectively.

LCMS: 99%, RT=1.03 min., (M+H)⁺=399 (method P). SFC: RT=5.19 min., (M+H)⁺=399 (method F).

### Example 19

### Synthesis of ((S)-1-(4-fluorophenyl)-3,4-dihydroisoquinolin-2(1H)-yl)((2S,5S)-5-((2-hydroxyethyl)amino)tetrahydro-2H-pyran-2-yl)methanone (Compound 5033)

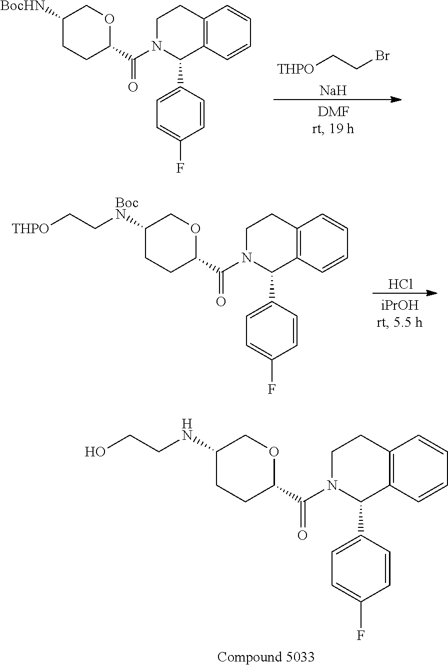

### Synthesis of tert-butyl ((3S,6S)-6-((S)-1-(4-fluorophenyl)-1,2,3,4-tetrahydroisoquinoline-2-carbonyl)tetrahydro-2H-pyran-3-yl)(2-methoxyethyl)carbamate

Starting from tert-butyl ((3S,6S)-6-((S)-1-(4-fluorophenyl)-1,2,3,4-tetrahydroisoquinoline-2-carbonyl)tetrahydro-2H-pyran-3-yl)carbamate (see Compound 5029, 75.7 mg, 0.167 mmol) and 2-(2-bromoethoxy)tetrahydro-2H-pyran (30.2 μl, 0.200 mmol), tert-butyl ((3S,6S)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate (see Compound 5030) and purified by acidic preparative MPLC (Linear Gradient: t=0 min 10% A; t=1 min 10% A; t=2 min 40% A; t=17 min 80% A; t=18 min 100%; t=23 min 100% A; detection: 220 nm).

LCMS: 100%, RT=2.34 min., (M+Na)⁺=605 (method A).

### Synthesis of ((S)-1-(4-fluorophenyl)-3,4-dihydroisoquinolin-2(1H)-yl)((2S,5S)-5-((2-hydroxyethyl)amino)tetrahydro-2H-pyran-2-yl)methanone (Compound 5033)

Starting from tert-butyl ((3S,6S)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate (21.1 mg, 0.036 mmol), ((S)-1-(4-fluorophenyl)-3,4-dihydroisoquinolin-2(1H)-yl)((2S,5S)-5-((2-hydroxyethyl)amino)tetrahydro-2H-pyran-2-yl)methanone (Compound 5033) was prepared as described for ((2R,5S)-5-aminotetrahydro-2H-pyran-2-yl)((S)-1-(4-fluorophenyl)-3,4-dihydroisoquinolin-2(1H)-yl)methanone (Compound 5042).

LCMS: 99%, RT=2.57 min., (M+H)⁺=399 (method AK).

### Example 20

### Synthesis of ((S)-1-(4-fluorophenyl)-3,4-dihydroisoquinolin-2(1H)-yl)((2R,5S)-5-((2-hydroxyethyl)amino)tetrahydro-2H-pyran-2-yl)methanone (Compound 5034)

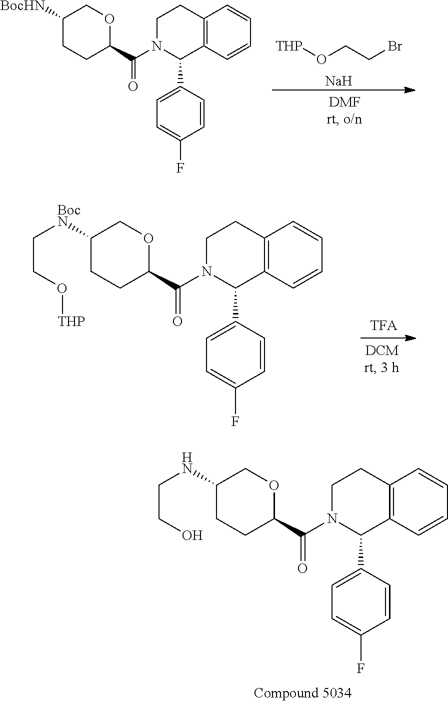

### Synthesis of tert-butyl ((3S,6R)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate

Starting from tert-butyl ((3S,6R)-6-((S)-1-(4-fluorophenyl)-1,2,3,4-tetrahydroisoquinoline-2-carbonyl)tetrahydro-2H-pyran-3-yl)carbamate (See Compound 5042, 100 mg, 0.220 mmol) and 2-(2-bromoethoxy)tetrahydro-2H-pyran (60 μL, 0.396 mmol), tert-butyl ((3S,6R)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate (see Compound 5030) and purified by basic preparative MPLC (Linear Gradient: t=0 min 10% A; t=1 min 10% A; t=2 min 40% A; t=17 min 80% A; t=18 min 100% A; t=23 min 100% A; detection: 220 nm).

LCMS: 99%, RT=2.27 min., (M-THP+H)⁺=499 (method A).

### Synthesis of ((S)-1-(4-fluorophenyl)-3,4-dihydroisoquinolin-2(1H)-yl)((2R,5S)-5-((2-hydroxyethyl)amino)tetrahydro-2H-pyran-2-yl)methanone (Compound 5034)

Starting from tert-butyl ((3S,6R)-6-((S)-1-(4-fluorophenyl)-1,2,3,4-tetrahydroisoquinoline-2-carbonyl)tetrahydro-2H-pyran-3-yl)(2-((tetrahydro-2H-pyran-2-yl)oxy)ethyl)carbamate (68 mg, 0.117 mmol), ((S)-1-(4-fluorophenyl)-3,4-dihydroisoquinolin-2(1H)-yl)((2R,5S)-5-((2-hydroxyethyl)amino)tetrahydro-2H-pyran-2-yl)methanone (Compound 5034) was prepared as described for ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030).

LCMS: 99%, RT=1.03 min., (M+H)⁺=399 (method P). SFC: RT=4.34 min., (M+H)⁺=399 (method AD).

### Example 21

### Synthesis of ((S)-1-(4-fluorophenyl)-3,4-dihydroisoquinolin-2(1H)-yl)((2S,5R)-5-((2-methoxyethyl)amino)tetrahydro-2H-pyran-2-yl)methanone (Compound 5035)

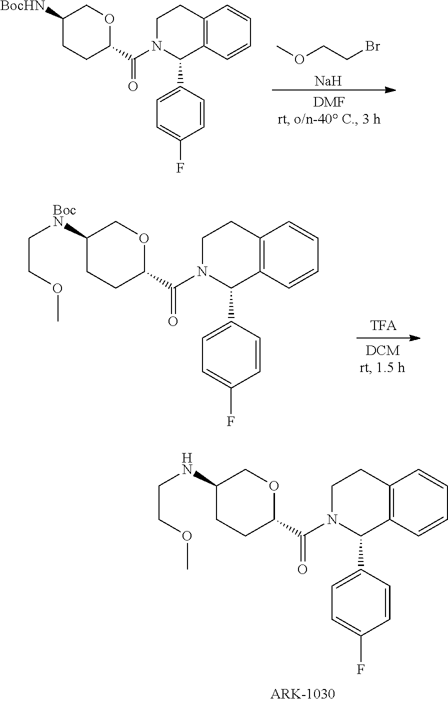

### Synthesis of tert-butyl ((3R,6S)-6-((S)-1-(4-fluorophenyl)-1,2,3,4-tetrahydroisoquinoline-2-carbonyl)tetrahydro-2H-pyran-3-yl)(2-methoxyethyl)carbamate

Starting from tert-butyl ((3R,6S)-6-((S)-1-(4-fluorophenyl)-1,2,3,4-tetrahydroisoquinoline-2-carbonyl)tetrahydro-2H-pyran-3-yl)carbamate (See Compound 5015, 100 mg, 0.209 mmol), tert-butyl ((3R,6S)-6-((S)-1-(4-fluorophenyl)-1,2,3,4-tetrahydroisoquinoline-2-carbonyl)tetrahydro-2H-pyran-3-yl)(2-methoxyethyl)carbamate was prepared as described for tert-butyl ((3S,6R)-6-((S)-1-(4-fluorophenyl)-1,2,3,4-tetrahydroisoquinoline-2-carbonyl)tetrahydro-2H-pyran-3-yl)(2-methoxyethyl)carbamate (see Compound 5030) and directly used in the next step.

LCMS: 70%, RT=2.21 min., (M-tBu)⁺=455 (method A).

### Synthesis of ((S)-1-(4-fluorophenyl)-3,4-dihydroisoquinolin-2(1H)-yl)((2S,5R)-5-((2-methoxyethyl)amino)tetrahydro-2H-pyran-2-yl)methanone (Compound 5035)

Starting from tert-butyl ((3R,6S)-6-((S)-1-(4-fluorophenyl)-1,2,3,4-tetrahydroisoquinoline-2-carbonyl)tetrahydro-2H-pyran-3-yl)(2-methoxyethyl)carbamate (99 mg (70 wt %)), 0.135 mmol), ((S)-1-(4-fluorophenyl)-3,4-dihydroisoquinolin-2(1H)-yl)((2S,5R)-5-((2-methoxyethyl)amino)tetrahydro-2H-pyran-2-yl)methanone (Compound 5035) was prepared as described for ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030).

LCMS: 99%, RT=1.07 min., (M+H)⁺=413 (method P). SFC: RT=4.33 min., (M+H)⁺=413 (method F).

### Example 22

### Synthesis of ((2R,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5036)

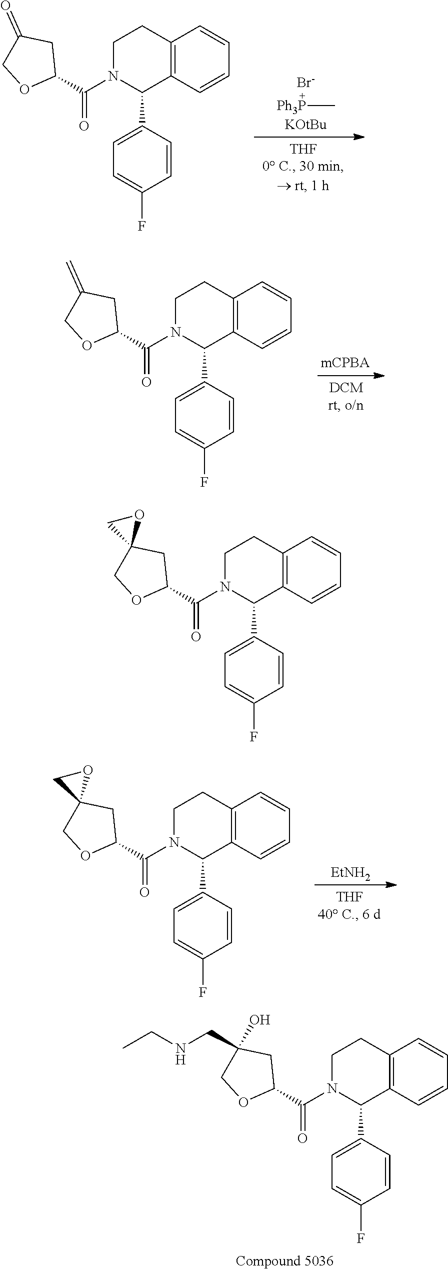

### Synthesis of ((S)-1-(4-fluorophenyl)-3,4-dihydroisoquinolin-2(1H)-yl)((R)-4-methylenetetrahydrofuran-2-yl)methanone

Under nitrogen atmosphere at 0° C., a solution of potassium tert-butoxide (1 M solution in tetrahydrofuran, 2.53 mL, 2.53 mmol) was added dropwise to a suspension of methyltriphenylphosphonium bromide (1054 mg, 2.95 mmol) in tetrahydrofuran (dry, 15.0 mL). After 30 minutes, a solution of (R)-5-((S)-1-(4-fluorophenyl)-1,2,3,4-tetrahydroisoquinoline-2-carbonyl)dihydrofuran-3(2H)-one (see Compound 5004, 715 mg, 2.107 mmol) in tetrahydrofuran (dry, 7.5 mL) was added dropwise. The reaction mixture was allowed to warm to room temperature and stirred for 1 hour. The mixture was filtered through Celite and the filtrate concentrated to dryness under reduced pressure. The residue was purified by flash column chromatography (silica, 0 to 30% ethyl acetate in heptane) to give ((S)-1-(4-fluorophenyl)-3,4-dihydroisoquinolin-2(1H)-yl)((R)-4-methylenetetrahydrofuran-2-yl)methanone.

LCMS: 99%, RT=2.14 min., (M+H)⁺=338 (method B).

### Synthesis of ((S)-1-(4-fluorophenyl)-3,4-dihydroisoquinolin-2(1H)-yl)((3R,6R)-1,5-dioxaspiro[2.4]heptan-6-yl)methanone and ((S)-1-(4-fluorophenyl)-3,4-dihydroisoquinolin-2(1H)-yl)((3S,6R)-1,5-dioxaspiro[2.4]heptan-6-yl)methanone

m-Chloroperbenzoic acid (70%, 513 mg, 2.081 mmol) was added to a solution of ((S)-1-(4-fluorophenyl)-3,4-dihydroisoquinolin-2(1H)-yl)((R)-4-methylenetetrahydrofuran-2-yl)methanone (585 mg, 1.734 mmol) in dichloromethane (6.0 mL). After stirring the reaction overnight, the mixture was purified by flash column chromatography (silica, 0 to 60% ethyl acetate in heptane) to give ((S)-1-(4-fluorophenyl)-3,4-dihydroisoquinolin-2(1H)-yl)((3R,6R)-1,5-dioxaspiro[2.4]heptan-6-yl)methanone as the first eluting isomer and ((S)-1-(4-fluorophenyl)-3,4-dihydroisoquinolin-2(1H)-yl)((3S,6R)-1,5-dioxaspiro[2.4]heptan-6-yl)methanone as the second eluting isomer.

First eluting isomer: LCMS: 95%, RT=2.00 min., (M+H)⁺=354 (method A).

Second eluting isomer: LCMS: 96%, RT=1.97 min., (M+H)⁺=354 (method A).

### Synthesis of ((2R,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5036)

Ethylamine (2.0 M in tetrahydrofuran, 1.026 mL, 2.052 mmol) was added to a solution of ((S)-1-(4-fluorophenyl)-3,4-dihydroisoquinolin-2(1H)-yl)((3S,6R)-1,5-dioxaspiro[2.4]heptan-6-yl)methanone (58 mg, 0.164 mmol) in tetrahydrofuran (dry, 1.0 mL). The reaction vial was sealed and heated at 40° C. for 6 days. The reaction mixture was concentrated to dryness under reduced pressure and the residue purified by acidic preparative MPLC (Linear Gradient: t=0 min 5% A; t=1 min 5% A; t=16.6 min 40% A; t=17.6 min 100%; t=23.8 min 100% A; detection: 220 nm). Product containing fractions were pooled, basified with saturated aqueous NaHCO₃ (2.5 mL), and extracted with ethyl acetate (2×20 mL). The combined organics were washed with brine (15 mL), dried over Na₂SO₄, and evaporated under reduced pressure to give ((2R,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5036) after lyophilization from a mixture of acetonitrile and water (1:1, 4 mL).

LCMS: 99%, RT=1.05 min., (M+H)⁺=399 (method P).

### Example 23

### Synthesis of ((2R,4R)-4-((ethylamino)methyl)-4-hydroxytetrahydrofuran-2-yl)((S)-1-(4-fluorophenyl)-3,4-dihydroisoquinolin-2(1H)-yl)methanone (Compound 5037)

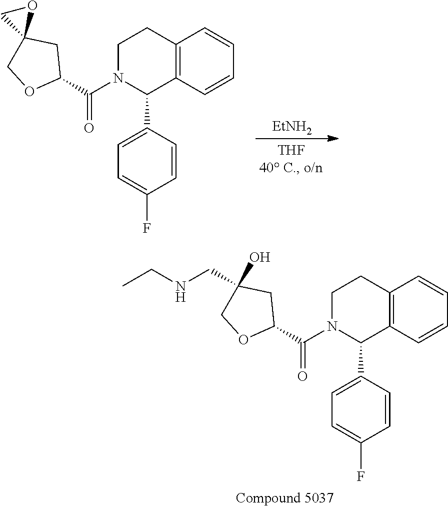

### Synthesis of ((2R,4R)-4-((ethylamino)methyl)-4-hydroxytetrahydrofuran-2-yl)((S)-1-(4-fluorophenyl)-3,4-dihydroisoquinolin-2(1H)-yl)methanone (Compound 5037)

Starting from ((S)-1-(4-fluorophenyl)-3,4-dihydroisoquinolin-2(1H)-yl)((3R,6R)-1,5-dioxaspiro[2.4]heptan-6-yl)methanone (see Compound 5036, 53 mg, 0.150 mmol), ((2R,4R)-4-((ethylamino)methyl)-4-hydroxytetrahydrofuran-2-yl)((S)-1-(4-fluorophenyl)-3,4-dihydroisoquinolin-2(1)-yl)methanone (Compound 5037) was prepared as described for ((2R,4S)-4-((ethylamino)methyl)-4-hydroxytetrahydrofuran-2-yl)((S)-1-(4-fluorophenyl)-3,4-dihydroisoquinolin-2(1H)-yl)methanone (Compound 5036).

LCMS: 97%, RT=1.03 min., (M+H)⁺=399 (method P).

### Example 24

### Synthesis of ((S)-1-(4-fluorophenyl)-3,4-dihydroisoquinolin-2(1H)-yl)((2S,4R)-4-hydroxy-4-(((2-hydroxyethyl)amino)methyl)tetrahydrofuran-2-yl)methanone (Compound 5038)

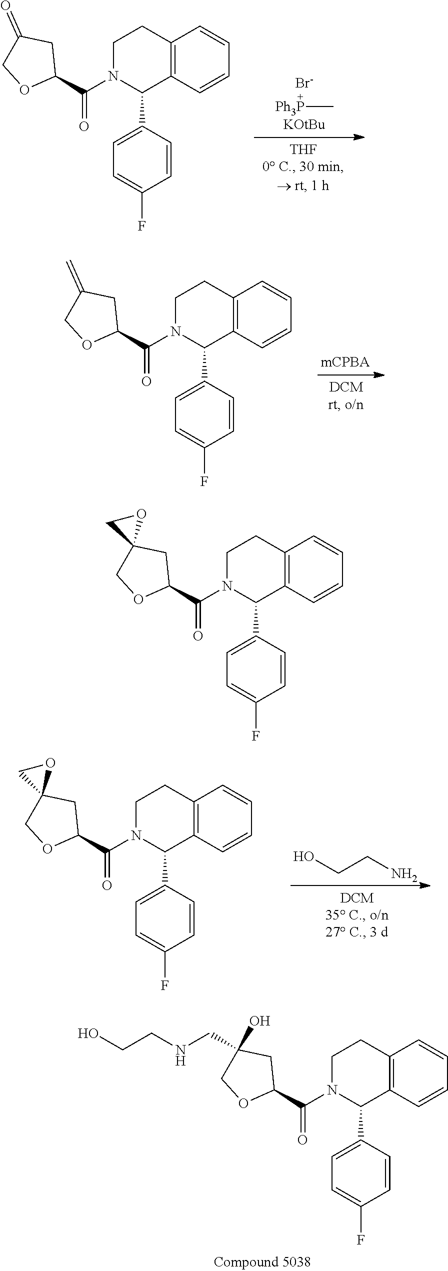

### Synthesis of ((S)-1-(4-fluorophenyl)-3,4-dihydroisoquinolin-2(1H)-yl)((S)-4-methylenetetrahydrofuran-2-yl)methanone

Starting from (R)-5-((S)-1-(4-fluorophenyl)-1,2,3,4-tetrahydroisoquinoline-2-carbonyl)dihydrofuran-3(2H)-one (see Compound 5004, 285 mg, 0.840 mmol), ((S)-1-(4-fluorophenyl)-3,4-dihydroisoquinolin-2(1H)-yl)((S)-4-methylenetetrahydrofuran-2-yl)methanone was prepared as described for ((S)-1-(4-fluorophenyl)-3,4-dihydroisoquinolin-2(1H)-yl)((R)-4-methylenetetrahydrofuran-2-yl)methanone (see Compound 5036).

LCMS: 99%, RT=2.17 min., (M+H)⁺=338 (method B).

### Synthesis of ((S)-1-(4-fluorophenyl)-3,4-dihydroisoquinolin-2(1H)-yl)((3S,6S)-1,5-dioxaspiro[2.4]heptan-6-yl)methanone and ((S)-1-(4-fluorophenyl)-3,4-dihydroisoquinolin-2(1H)-yl)((3R,6S)-1,5-dioxaspiro[2.4]heptan-6-yl)methanone

Starting from ((S)-1-(4-fluorophenyl)-3,4-dihydroisoquinolin-2(1H)-yl)((S)-4-methylenetetrahydrofuran-2-yl)methanone (145 mg, 0.430 mmol), ((S)-1-(4-fluorophenyl)-3,4-dihydroisoquinolin-2(1H)-yl)((3S,6S)-1,5-dioxaspiro[2.4]heptan-6-yl)methanone as the first eluting isomer and ((S)-1-(4-fluorophenyl)-3,4-dihydroisoquinolin-2(1H)-yl)((3R,6S)-1,5-dioxaspiro[2.4]heptan-6-yl)methanone as the second eluting isomer were prepared as described for ((S)-1-(4-fluorophenyl)-3,4-dihydroisoquinolin-2(1H)-yl)((3R,6R)-1,5-dioxaspiro[2.4]heptan-6-yl)methanone (see Compound 5036). The configuration of the spiro-epoxide was arbitrarily assigned

First eluting isomer: LCMS: 98%, RT=1.99 min., (M+H)⁺=354 (method A).

Second eluting isomer: LCMS: 99%, RT=1.97 min., (M+H)⁺=354 (method A).

### Synthesis of ((S)-1-(4-fluorophenyl)-3,4-dihydroisoquinolin-2(1H)-yl)((2S,4R)-4-hydroxy-4-(((2-hydroxyethyl)amino)methyl)tetrahydrofuran-2-yl)methanone (Compound 5038)

Starting from ((S)-1-(4-fluorophenyl)-3,4-dihydroisoquinolin-2(1H)-yl)((3R,6S)-1,5-dioxaspiro[2.4]heptan-6-yl)methanone (30 mg, 0.085 mmol), ((S)-1-(4-fluorophenyl)-3,4-dihydroisoquinolin-2(1H)-yl)((2S,4R)-4-hydroxy-4-(((2-hydroxyethyl)amino)methyl)tetrahydrofuran-2-yl)methanone (Compound 5038) was prepared as described for ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040). The configuration of the tetrahydrofuran-hydroxy moiety was arbitrarily assigned

LCMS: 97%, RT=1.02 min., (M+H)⁺=415 (method P).

### Example 25

### Synthesis of ((S)-1-(4-fluorophenyl)-3,4-dihydroisoquinolin-2(1H)-yl)((2S,4S)-4-hydroxy-4-(((2-hydroxyethyl)amino)methyl)tetrahydrofuran-2-yl)methanone (Compound 5039)

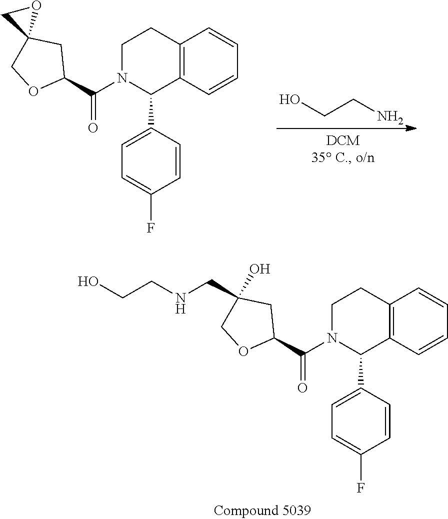

### Synthesis of ((S)-1-(4-fluorophenyl)-3,4-dihydroisoquinolin-2(1H)-yl)((2S,4S)-4-hydroxy-4-(((2-hydroxyethyl)amino)methyl)tetrahydrofuran-2-yl)methanone (Compound 5039)

Starting from ((S)-1-(4-fluorophenyl)-3,4-dihydroisoquinolin-2(1H)-yl)((3S,6S)-1,5-dioxaspiro[2.4]heptan-6-yl)methanone (see Compound 5038, 71 mg, 0.201 mmol), ((S)-1-(4-fluorophenyl)-3,4-dihydroisoquinolin-2(1H)-yl)((2S,4S)-4-hydroxy-4-(((2-hydroxyethyl)amino)methyl)tetrahydrofuran-2-yl)methanone (Compound 5039) was prepared as described for ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040) after lyophilization from a mixture of acetonitrile and water (1:1, 4 mL). The configuration of the tetrahydrofuran-hydroxy moiety was arbitrarily assigned

LCMS: 99%, RT=1.00 min., (M+H)⁺=415 (method P).

### Example 26

### Synthesis of ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040)

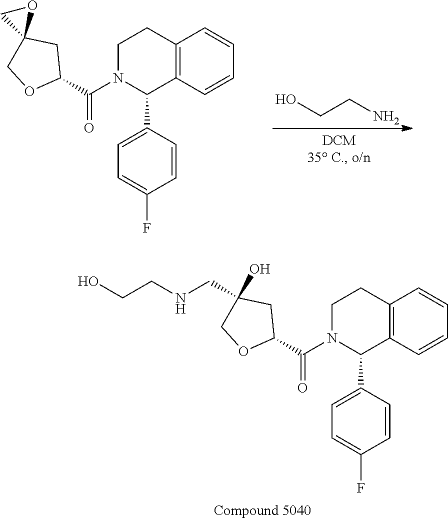

### Synthesis of ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040)

Ethanolamine (0.055 mL, 0.906 mmol) was added to a solution of ((S)-1-(4-fluorophenyl)-3,4-dihydroisoquinolin-2(1H)-yl)((3R,6R)-1,5-dioxaspiro[2.4]heptan-6-yl)methanone (see Compound 5036, 64 mg, 0.181 mmol) in dichloromethane (1.8 mL). The reaction vial was sealed and heated at 35° C. overnight. The reaction mixture was concentrated to dryness under reduced pressure and the residue purified by acidic preparative MPLC (Linear Gradient: t=0 min 5% A; t=1 min 5% A; t=16.6 min 40% A; t=17.6 min 100%; t=23.8 min 100% A; detection: 220 nm). Product containing fractions were pooled, basified with saturated aqueous NaHCO₃ (2.5 mL), and extracted with ethyl acetate (2×20 mL). The combined organics were washed with brine (15 mL), dried over Na₂SO₄, and evaporated under reduced pressure to give ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040) after lyophilization from a mixture of acetonitrile and water (1:1, 4 mL).

LCMS: 99%, RT=1.01 min., (M+H)⁺=415 (method P).

### Example 27

### Synthesis of ((S)-1-(4-fluorophenyl)-3,4-dihydroisoquinolin-2(1H)-yl)((2R,4S)-4-hydroxy-4-(((2-hydroxyethyl)amino)methyl)tetrahydrofuran-2-yl)methanone (Compound 5041)

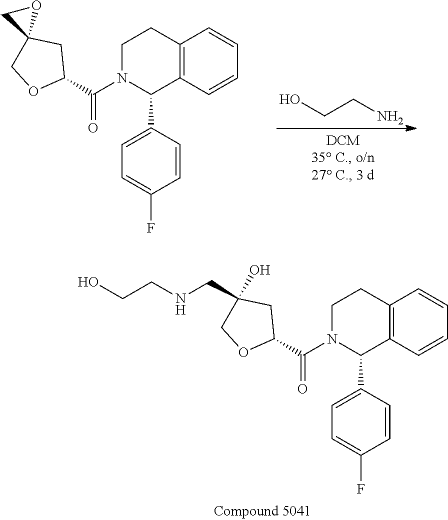

### Synthesis of ((S)-1-(4-fluorophenyl)-3,4-dihydroisoquinolin-2(1H)-yl)((2R,4S)-4-hydroxy-4-(((2-hydroxyethyl)amino)methyl)tetrahydrofuran-2-yl)methanone (Compound 5041)

Starting from ((S)-1-(4-fluorophenyl)-3,4-dihydroisoquinolin-2(1H)-yl)((3S,6R)-1,5-dioxaspiro[2.4]heptan-6-yl)methanone (see Compound 5036, 55 mg, 0.156 mmol), (S)-1-(4-fluorophenyl)-3,4-dihydroisoquinolin-2(1H)-yl)((2R,4S)-4-hydroxy-4-(((2-hydroxyethyl)amino)methyl)tetrahydrofuran-2-yl)methanone (Compound 5041) was prepared as described for ((S)-1-(4-fluorophenyl)-3,4-dihydroisoquinolin-2(1H)-yl)((2R,4R)-4-hydroxy-4-(((2-hydroxyethyl)amino)methyl)tetrahydrofuran-2-yl)methanone (Compound 5040).

LCMS: 99%, RT=1.02 min., (M+H)⁺=415 (method P).

### Example 28

### Synthesis of ((2R,5S)-5-aminotetrahydro-2H-pyran-2-yl)((S)-1-(4-fluorophenyl)-3,4-dihydroisoquinolin-2(1H)-yl)methanone (Compound 5042)

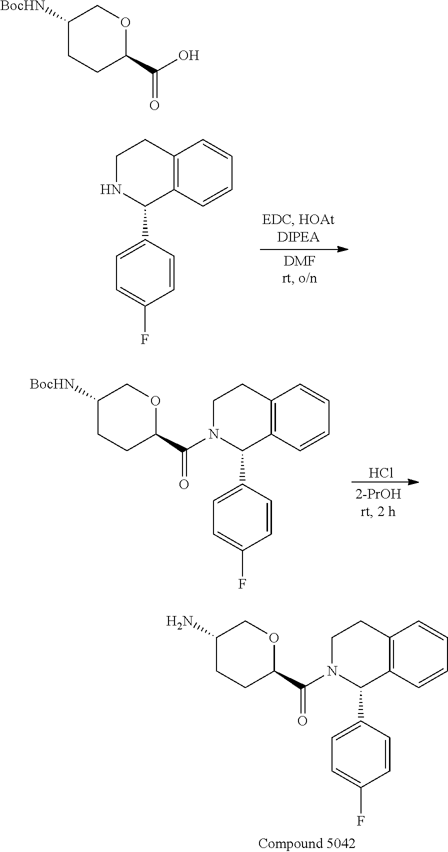

### Synthesis of tert-butyl ((3S,6R)-6-((S)-1-(4-fluorophenyl)-1,2,3,4-tetrahydroisoquinoline-2-carbonyl)tetrahydro-2H-pyran-3-yl)carbamate

Under nitrogen atmosphere, (S)-1-(4-fluorophenyl)-1,2,3,4-tetrahydroisoquinoline (281 mg, 1.237 mmol) and N,N-diisopropylethylamine (0.514 mL, 2.95 mmol) were added to a suspension of (2R,5S)-5-((tert-butoxycarbonyl)amino)tetrahydro-2H-pyran-2-carboxylic acid (289 mg, 1.178 mmol), N-(3-dimethylaminopropyl)-N′-ethylcarbodiimide hydrochloride (248 mg, 1.296 mmol), and 1-hydroxy-7-azabenzotriazole (32.1 mg, 0.236 mmol) in N,N-dimethylformamide (dry, 5.5 mL) and the mixture was stirred overnight. The reaction mixture was diluted with ethyl acetate (50 mL) and washed with brine (15 mL). The aqueous layer was extracted with ethyl acetate (20 mL) and the combined organic phase was washed with brine (3×15 mL), dried over Na₂SO₄, and evaporated under reduced pressure. The residue was purified by flash column chromatography (silica, 0 to 45% ethyl acetate in heptane) to give tert-butyl ((3S,6R)-6-((S)-1-(4-fluorophenyl)-1,2,3,4-tetrahydroisoquinoline-2-carbonyl)tetrahydro-2H-pyran-3-yl)carbamate.

LCMS: 99%, RT=2.16 min., (M+H)⁺=455 (method A).

### Synthesis of ((2R,5S)-5-aminotetrahydro-2H-pyran-2-yl)((S)-1-(4-fluorophenyl)-3,4-dihydroisoquinolin-2(1H)-yl)methanone (Compound 5042)

HCl (5-6 M in 2-propanol, 4.5 mL, 112 mmol) was added to tert-butyl ((3S,6R)-6-((S)-1-(4-fluorophenyl)-1,2,3,4-tetrahydroisoquinoline-2-carbonyl)tetrahydro-2H-pyran-3-yl)carbamate (694 mg, 1.526 mmol). After 2 hours, the reaction mixture was diluted with dichloromethane (25 mL) and washed with saturated aqueous Na₂CO₃ (10 mL). The aqueous layer was extracted with dichloromethane (3×15 mL) and the combined organic phase was dried over Na₂SO₄ and evaporated under reduced pressure. The residue was dissolved in methanol and brought onto an SCX-2 column (5 g) and eluted with methanol until neutral. Next, the column was eluted with ammonia in methanol (1 M). The basic fraction was concentrated to dryness under reduced pressure. The residue was lyophilized from a mixture of acetonitrile and water (1:2, 30 mL) to give ((2R,5S)-5-aminotetrahydro-2H-pyran-2-yl)((S)-1-(4-fluorophenyl)-3,4-dihydroisoquinolin-2(1H)-yl)methanone (Compound 5042).

LCMS: 99%, RT=1.03 min., (M+H)⁺=355 (method P). SFC: RT=5.32 min., (M+H)⁺=355 (method W).

### Example 29

### Synthesis of ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043)

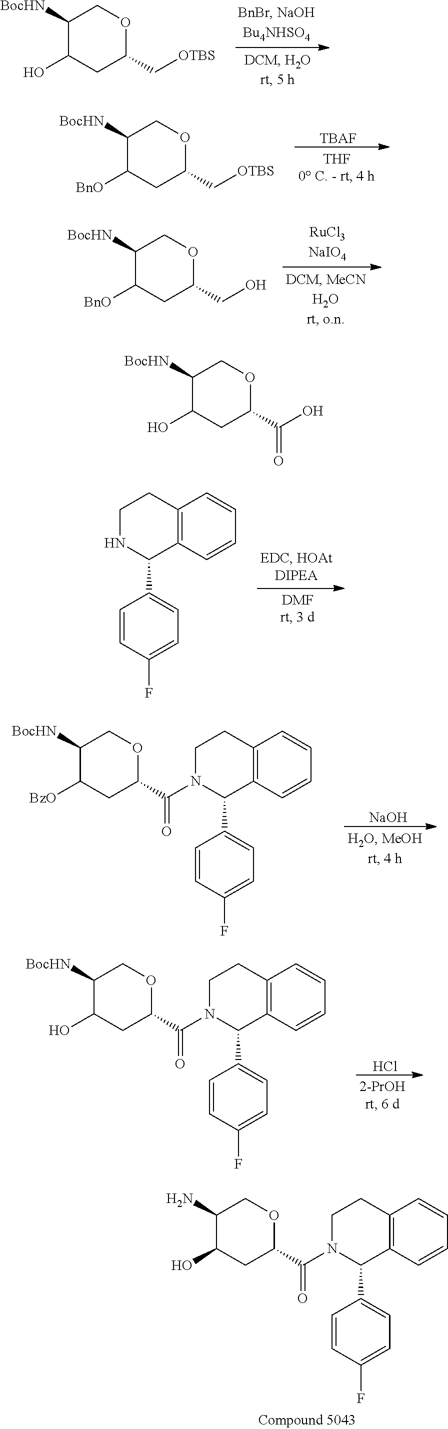

### Synthesis of tert-butyl ((3S,6S)-4-(benzyloxy)-6-(((tert-butyldimethylsilyl)oxy)methyl)tetrahydro-2H-pyran-3-yl)carbamate

Aqueous NaOH (50%, 20 mL) was added to a solution of tert-butyl ((3S,6S)-6-(((tert-butyldimethylsilyl)oxy)methyl)-4-hydroxytetrahydro-2H-pyran-3-yl)carbamate (0.70 g, 1.936 mmol), tetrabutylammonium hydrogensulfate (1.972 g, 5.81 mmol), and benzyl bromide (0.576 mL, 4.84 mmol) in dichloromethane (20 mL). The mixture was stirred for 5 hours, diluted with ice cold water (50 mL) and extracted with dichloromethane (3×30 mL). The combined phase was dried over Na₂SO₄ and evaporated under reduced pressure. The residue was purified by flash column chromatography (silica, 0 to 35% ethyl acetate in heptane) to give tert-butyl ((3S,6S)-4-(benzyloxy)-6-(((tert-butyldimethylsilyl)oxy)methyl)tetrahydro-2H-pyran-3-yl)carbamate.

LCMS: 85%, RT=2.69 min., (M-Boc+H)⁺=352 (method B).

### Synthesis of tert-butyl ((3S,6S)-4-(benzyloxy)-6-(hydroxymethyl)tetrahydro-2H-pyran-3-yl)carbamate

Under argon atmosphere at 0° C., tetrabutylammonium fluoride (1.0 M solution in tetrahydrofuran, 4.14 mL, 4.14 mmol) was added dropwise to a solution of tert-butyl ((3S,6S)-4-(benzyloxy)-6-(((tert-butyldimethylsilyl)oxy)methyl)tetrahydro-2H-pyran-3-yl)carbamate (0.623 g, 1.379 mmol) in tetrahydrofuran (dry, 10 mL). The reaction mixture was allowed to reach room temperature and stirred for 4 hours. The reaction mixture was quenched with saturated aqueous NH₄Cl (50 mL) and extracted with ethyl acetate (3×20 mL). The combined organic phase was washed with brine (20 mL), dried over Na₂SO₄, and evaporated under reduced pressure. The residue was purified by flash column chromatography (silica, 0 to 80% ethyl acetate in heptane) to give tert-butyl ((3S,6S)-4-(benzyloxy)-6-(hydroxymethyl)tetrahydro-2H-pyran-3-yl)carbamate.

LCMS: 95%, RT=1.91 min., (M+Na)⁺=360 (method A

### Synthesis of (2S,5S)-4-(benzoyloxy)-5-((tert-butoxycarbonyl)amino)tetrahydro-2H-pyran-2-carboxylic acid

Sodium periodate (875 mg, 4.09 mmol) and ruthenium(III) chloride hydrate (30.7 mg, 0.136 mmol) were added to a solution of tert-butyl ((3S,6S)-4-(benzyloxy)-6-(hydroxymethyl)tetrahydro-2H-pyran-3-yl)carbamate (460 mg, 1.363 mmol) in a mixture of dichloromethane (6 mL), acetonitrile (6 mL) and water (9 mL). After stirring overnight, the mixture was cooled in an ice/water bath and diluted with saturated aqueous Na₂S₂O₃. After warming to room temperature, the pH was adjusted to ˜6-7 by adding aqueous HCl (1 M) and the mixture was extracted with ethyl acetate (3×25 mL). The combined organic phase was washed with brine (10 mL), dried over Na₂SO₄, and evaporated under reduced pressure. The residue was purified by acidic preparative MPLC (Linear Gradient: t=0 min 5% A, t=1 min 5% A; t=16 min 60% A; t=17 min 100%; t=22 min 100% A; detection: 220 nm) to give (2S,5S)-4-(benzoyloxy)-5-((tert-butoxycarbonyl)amino)tetrahydro-2H-pyran-2-carboxylic acid.

LCMS: 68%, RT=1.88 min., (M−H)⁻=364 (method A).

### Synthesis of (2S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl benzoate

Under an argon atmosphere, N-(3-dimethylaminopropyl)-N′-ethylcarbodiimide hydrochloride (23.91 mg, 0.125 mmol) and 1-hydroxy-7-azabenzotriazole (3.23 mg, 0.024 mmol) followed by (S)-1-(4-fluorophenyl)-1,2,3,4-tetrahydroisoquinoline (27 mg, 0.119 mmol) and N,N-diisopropylethylamine (0.052 mL, 0.297 mmol) were added to a solution of (2S,5S)-4-(benzoyloxy)-5-((tert-butoxycarbonyl)amino)tetrahydro-2H-pyran-2-carboxylic acid (43.4 mg, 0.119 mmol) in N,N-dimethylformamide (dry, 1 mL). After stirring for 3 days, the mixture was diluted with ethyl acetate (10 mL) and washed with saturated aqueous NaHCO₃ (5 mL). The aqueous phase was extracted with ethyl acetate (3×10 mL) and the combined organics were washed with brine (15 mL), dried over Na₂SO₄, and evaporated under reduced pressure. The residue was purified by flash column chromatography (silica, 0 to 50% ethyl acetate in heptane) to give (2S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl benzoate.

LCMS: 95%, RT=2.25 min., (M+H)⁺=575 (method A).

### Synthesis of tert-butyl ((3S,6S)-6-((S)-1-(4-fluorophenyl)-1,2,3,4-tetrahydroisoquinoline-2-carbonyl)-4-hydroxytetrahydro-2H-pyran-3-yl)carbamate

Aqueous NaOH (1 M, 0.139 mL, 0.139 mmol) was added to a solution of (2S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl benzoate (40 mg, 0.070 mmol) in a mixture of water and methanol (1:1, 0.5 mL). After 4 hours, the reaction mixture was concentrated under reduced pressure. The residue was dissolved in water (5 mL) and extracted with dichloromethane (2×10 mL). The organic layers were combined and passed through a phase-separator. The filtrate was evaporated under reduced pressure. The residue was purified by acidic preparative MPLC (Linear Gradient: t=0 min 5% A, t=1 min 5% A; t=16 min 60% A; t=17 min 100%; t=22 min 100% A; detection: 220/254 nm) to give tert-butyl ((3S,6S)-6-((S)-1-(4-fluorophenyl)-1,2,3,4-tetrahydroisoquinoline-2-carbonyl)-4-hydroxytetrahydro-2H-pyran-3-yl)carbamate.

LCMS: 100%, RT=2.07 min., (M+H)⁺=471 (method A).

### Synthesis of ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043)

HCl (5-6 M in 2-propanol, 83 μL, 0.46 mmol) was added to a solution of tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (20 mg, 0.043 mmol) in 2-propanol (0.5 mL) and stirred overnight. Three portions of HCl (5-6 M in 2-propanol, 83 μL, 0.46 mmol) were added after stirring for respectively one, two, and three days. After further stirring for three days, the reaction mixture was diluted with dichloromethane (10 mL) and saturated aqueous K₂CO₃ (5 mL). The layers were separated and the aqueous phase was extracted with dichloromethane (3×10 mL). The combined phase was dried over Na₂SO₄ and evaporated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure and lyophilized from a mixture of acetonitrile and water (1:1, 4 mL) to give ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 100%, RT=1.01 min., (M+H)⁺=371 (method P).

### Example 30

### Synthesis of ((S)-1-(4-fluorophenyl)-3,4-dihydroisoquinolin-2(1H)-yl)((S)-1,4-oxazepan-7-yl)methanone (Compound 5044) and ((S)-1-(4-fluorophenyl)-3,4-dihydroisoquinolin-2(1H)-yl)((R)-1,4-oxazepan-7-yl)methanone (Compound 5045)

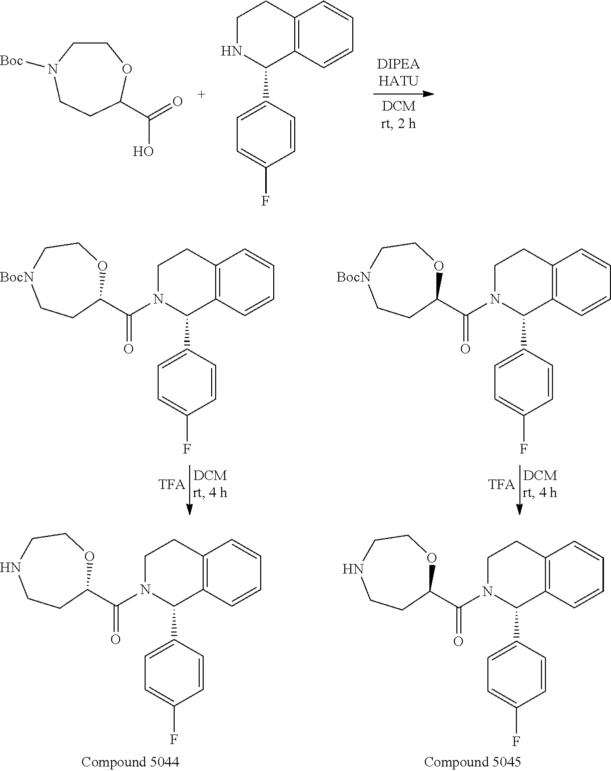 [SMILES: CC(C)(C)OC(=O)N1CCOC(C(=O)O)CC1 ; Fc1ccc([C@@H]2NCCc3ccccc32)cc1 ; CC(C)(C)OC(=O)N1CCO[C@H](C(=O)N2CCc3ccccc3[C@@H]2c2ccc(F)cc2)CC1 ; CC(C)(C)OC(=O)N1CCO[C@@H](C(=O)N2CCc3ccccc3[C@@H]2c2ccc(F)cc2)CC1 ; O=C([C@@H]1CCNCCO1)N1CCc2ccccc2[C@@H]1c1ccc(F)cc1 ; O=C([C@H]1CCNCCO1)N1CCc2ccccc2[C@@H]1c1ccc(F)cc1]

### Synthesis of tert-butyl (S)-7-((S)-1-(4-fluorophenyl)-1,2,3,4-tetrahydroisoquinoline-2-carbonyl)-1,4-oxazepane-4-carboxylate and (R)-7-((S)-1-(4-fluorophenyl)-1,2,3,4-tetrahydroisoquinoline-2-carbonyl)-1,4-oxazepane-4-carboxylate

N,N-diisopropylethylamine (80 μl, 0.459 mmol) and 1-[bis(dimethylamino)methylene]-1H-1,2,3-triazolo[4,5-b]pyridinium 3-oxid hexafluorophosphate (153 mg, 0.404 mmol) were added to a solution of 4-(tert-butoxycarbonyl)-1,4-oxazepane-7-carboxylic acid (90 mg, 0.367 mmol) in dichloromethane (5 mL). After 10 minutes, (S)-1-(4-fluorophenyl)-1,2,3,4-tetrahydroisoquinoline (92 mg, 0.404 mmol) was added and the mixture was stirred for 2 hours. Then, the reaction mixture was washed with aqueous HCl (1 M, 5 mL), saturated aqueous NaHCO₃ (5 mL), and brine (5 mL), dried over Na₂SO₄, and evaporated under reduced pressure. The residue was purified by preparative LCMS (method BF) and by preparative chiral SFC (method Y) to give tert-butyl (S)-7-((S)-1-(4-fluorophenyl)-1,2,3,4-tetrahydroisoquinoline-2-carbonyl)-1,4-oxazepane-4-carboxylate as the first eluting SFC isomer and tert-butyl (R)-7-((S)-1-(4-fluorophenyl)-1,2,3,4-tetrahydroisoquinoline-2-carbonyl)-1,4-oxazepane-4-carboxylate as the second eluting SFC isomer. The configuration of the oxazepane stereocenter was arbitrarily assigned.

First eluting SFC isomer: LCMS: 97%, RT=2.18 min., (M-tBu+H)⁺=399 (method A). SFC: RT=2.60 min., (M-tBu+H)⁺=399 (method W).

Second eluting SFC isomer: LCMS: 99%, RT=2.17 min., (M-tBu+H)⁺=399 (method A). SFC: RT=3.14 min., (M-tBu+H)⁺=399 (method W).

### Synthesis of ((S)-1-(4-fluorophenyl)-3,4-dihydroisoquinolin-2(1H)-yl)((S)-1,4-oxazepan-7-yl)methanone (Compound 5044)

Trifluoroacetic acid (0.5 mL, 6.53 mmol) was added to a solution of tert-butyl (S)-7-((S)-1-(4-fluorophenyl)-1,2,3,4-tetrahydroisoquinoline-2-carbonyl)-1,4-oxazepane-4-carboxylate (10 mg, 0.022 mmol) in dichloromethane (5 mL). After 4 hours, the reaction mixture was concentrated to dryness under reduced pressure. The residue was dissolved in methanol (1 mL) and brought onto a SCX-2 column (1 g) and eluted with methanol until neutral. Next, the column was eluted with ammonia in methanol (3 M). The basic fraction was concentrated to dryness under reduced pressure to give ((S)-1-(4-fluorophenyl)-3,4-dihydroisoquinolin-2(1H)-yl)((S)-1,4-oxazepan-7-yl)methanone (Compound 5044) after lyophilization from a mixture of acetonitrile and water (1:1, 4 mL). The configuration of the oxazepane stereocenter was arbitrarily assigned.

LCMS: 100%, RT=1.05 min., (M+H)⁺=355 (method P). SFC: RT=4.16 min., (M+H)⁺=355 (method W).

### Synthesis of ((S)-1-(4-fluorophenyl)-3,4-dihydroisoquinolin-2(1H)-yl)((R)-1,4-oxazepan-7-yl)methanone (Compound 5045)

Starting from tert-butyl (R)-7-((S)-1-(4-fluorophenyl)-1,2,3,4-tetrahydroisoquinoline-2-carbonyl)-1,4-oxazepane-4-carboxylate (10 mg, 0.022 mmol), ((S)-1-(4-fluorophenyl)-3,4-dihydroisoquinolin-2(1H)-yl)((R)-1,4-oxazepan-7-yl)methanone (Compound 5045) was prepared as described for ((S)-1-(4-fluorophenyl)-3,4-dihydroisoquinolin-2(1H)-yl)((S)-1,4-oxazepan-7-yl)methanone (Compound 5044). The configuration of the oxazepane stereocenter was arbitrarily assigned.

LCMS: 100%, RT=1.04 min., (M+H)⁺=355 (method P). SFC: RT=4.63 min., (M+H)⁺=355 (method W).

### Example 31

### Synthesis of ((2S,5S)-5-(aminomethyl)-5-hydroxytetrahydro-2H-pyran-2-yl)((S)-1-(4-fluorophenyl)-3,4-dihydroisoquinolin-2(1H)-yl)methanone (Compound 5046) and ((2R,5R)-5-(aminomethyl)-5-hydroxytetrahydro-2H-pyran-2-yl)((S)-1-(4-fluorophenyl)-3,4-dihydroisoquinolin-2(1H)-yl)methanone (Compound 5047)

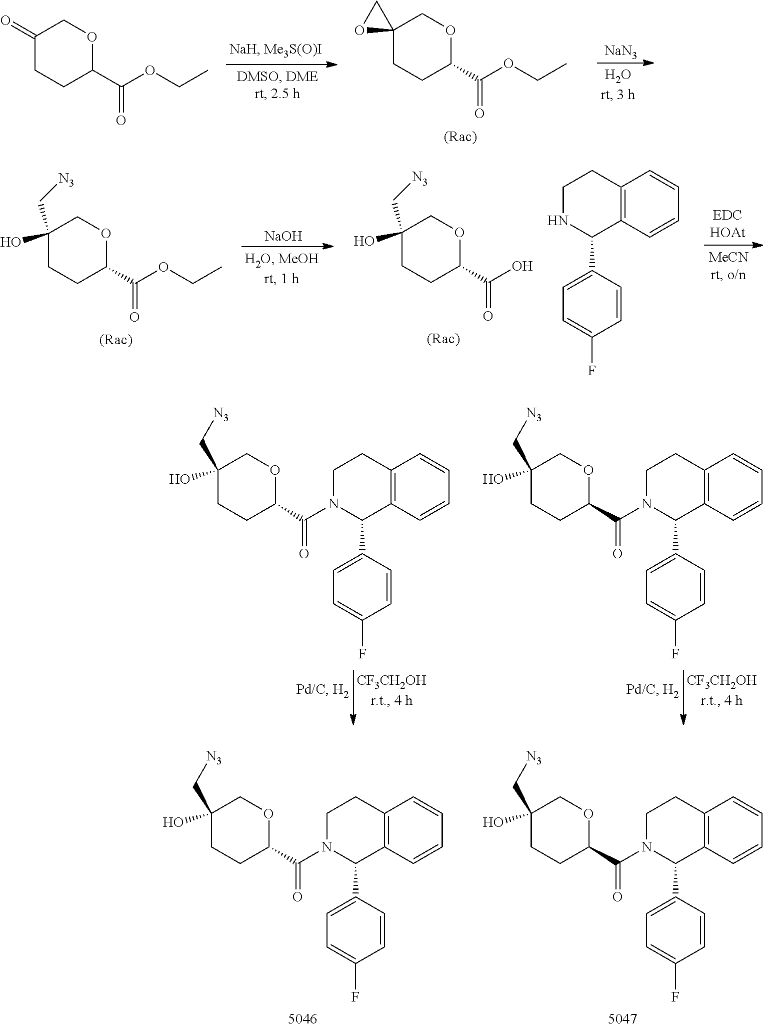

### Synthesis of ethyl trans-1,5-dioxaspiro[2.5]octane-6-carboxylate

Under nitrogen atmosphere, sodium hydride (60% dispersion in oil, 32.5 mg, 0.813 mmol) was added to a solution of trimethylsulfoxonium iodide (179 mg, 0.813 mmol) in dimethyl sulfoxide (2.0 mL). After 0.5 hour, a solution of ethyl 5-oxotetrahydro-2H-pyran-2-carboxylate (100 mg, 0.581 mmol) in 1,2-dimethoxyethane (1.0 mL) was added. After stirring for another 2 hours, the reaction mixture was poured into a mixture of saturated aqueous NH₄Cl and ice (20 mL) and the resulting mixture was extracted with diethyl ether (2×15 mL) and ethyl acetate (15 mL). The combined organic phase was washed with water, dried over Na₂SO₄ and concentrated under reduced pressure. The residue was purified by flash column chromatography (silica, 10 to 50% ethyl acetate in heptane) to give ethyl trans-1,5-dioxaspiro[2.5]octane-6-carboxylate.

GCMS: 99%, RT=3.06 min., (M-CO₂Et)⁺=113 (method A20).

### Synthesis of ethyl trans-5-(azidomethyl)-5-hydroxytetrahydro-2H-pyran-2-carboxylate

A mixture of sodium azide (52.4 mg, 0.806 mmol) in acetic acid (0.15 mL) and water (0.26 mL) was added to ethyl trans-1,5-dioxaspiro[2.5]octane-6-carboxylate (30 mg, 0.161 mmol). After 3 hours, the reaction mixture was diluted with dichloromethane (10 mL) and a mixture of saturated aqueous K₂CO₃ (1 mL) and water (1 mL). The layers were separated using a phase separator and the organic filtrate was concentrated. The residue was purified by flash column chromatography (silica, 10 to 70% ethyl acetate in heptane) to give ethyl trans-5-(azidomethyl)-5-hydroxytetrahydro-2H-pyran-2-carboxylate.

LCMS: 99%, RT=1.76 min. (method B).

### Synthesis of trans-5-(azidomethyl)-5-hydroxytetrahydro-2H-pyran-2-carboxylic acid

Aqueous NaOH (2 M, 0.079 mL, 0.157 mmol) was added to a solution of ethyl trans-5-(azidomethyl)-5-hydroxytetrahydro-2H-pyran-2-carboxylate (30 mg, 0.131 mmol) in methanol (0.3 mL). After 1 hour, the mixture was diluted with dichloromethane and acidified to pH ˜ 1-2 with aqueous HCl (1 M). The layers were separated using a phase separator and the water layer was treated with brine (4 mL) and stirred with a mixture of dichloromethane and methanol (4:1, 4 mL). The layers were separated using a phase separator and the organic filtrate was combined with the organic filtrate of the first extraction and evaporated under reduced pressure to give trans-5-(azidomethyl)-5-hydroxytetrahydro-2H-pyran-2-carboxylic acid.

### Synthesis of (trans-5-(azidomethyl)-5-hydroxytetrahydro-2H-pyran-2-yl)((S)-1-(4-fluorophenyl)-3,4-dihydroisoquinolin-2(1H)-yl)methanone

1-Hydroxy-7-azabenzotriazole (2.57 mg, 0.019 mmol) and N-(3-dimethylaminopropyl)-N′-ethylcarbodiimide hydrochloride (21.73 mg, 0.113 mmol) were added to a solution of (S)-1-(4-fluorophenyl)-1,2,3,4-tetrahydroisoquinoline (21.46 mg, 0.094 mmol) and trans-5-(azidomethyl)-5-hydroxytetrahydro-2H-pyran-2-carboxylic acid (19 mg, 0.094 mmol) in acetonitrile (0.5 mL). After stirring overnight, the mixture was diluted with water and acetonitrile (1:1, 1 mL) and purified by acidic preparative MPLC (Linear Gradient: t=0 min 20% A; t=3 min 20% A; t=18 min 60% A; t=19 min 100%; t=24 min 100% A; detection: 210 nm). The product containing fractions were pooled and the volatiles were removed under reduced pressure. The aqueous residue was diluted with saturated aqueous NaHCO₃ (5 mL) and dichloromethane (5 mL) and stirred for 2 hours. The layers were separated using a phase separator and the organic filtrate was evaporated under reduced pressure. The residue was purified by preparative chiral SFC to give ((2S,5S)-5-(azidomethyl)-5-hydroxytetrahydro-2H-pyran-2-yl)((S)-1-(4-fluorophenyl)-3,4-dihydroisoquinolin-2(1H)-yl)methanone as the first eluting SFC isomer and ((2R,5R)-5-(azidomethyl)-5-hydroxytetrahydro-2H-pyran-2-yl)((S)-1-(4-fluorophenyl)-3,4-dihydroisoquinolin-2(1H)-yl)methanone as the second eluting SFC isomer. The configuration of the pyran stereocenters is arbitrarily assigned.

First eluting isomer: LCMS: 99%, RT=2.07 min., (M+H)⁺=411 (method C). SFC: RT=3.73 min., (M+H)⁺=411 (method W).

Second eluting isomer: LCMS: 99%, RT=2.07 min., (M+H)⁺=411 (method C). SFC: RT=4.94 min., (M+H)⁺=411 (method W).

### Synthesis of ((2S,5S)-5-(aminomethyl)-5-hydroxytetrahydro-2H-pyran-2-yl)((S)-1-(4-fluorophenyl)-3,4-dihydroisoquinolin-2(1H)-yl)methanone (Compound 5046)

Palladium on carbon (10 wt %, containing 50% water, spatula point) was added to a solution of ((2S,5S)-5-(azidomethyl)-5-hydroxytetrahydro-2H-pyran-2-yl)((S)-1-(4-fluorophenyl)-3,4-dihydroisoquinolin-2(1H)-yl)methanone (12.2 mg, 0.030 mmol). The mixture was hydrogenated at room temperature and ambient pressure for 4 hours, diluted with methanol, filtered through a nylon, 0.45 μm filter and the filtrate was evaporated under reduced pressure. The residue was purified by acidic preparative MPLC (Linear Gradient: t=0 min 5% A; t=2 min 10% A; t=17 min 50% A; t=18 min 100%; t=23 min 100% A; detection: 210 nm). The product containing fractions were pooled, diluted with saturated aqueous NaHCO₃ (4 mL), and extracted with dichloromethane. The organic layer was passed through a phase separator and evaporated under reduced pressure to give ((2S,5S)-5-(aminomethyl)-5-hydroxytetrahydro-2H-pyran-2-yl)((S)-1-(4-fluorophenyl)-3,4-dihydroisoquinolin-2(1H)-yl)methanone (Compound 5046) after lyophilization from a mixture of acetonitrile and water (1:1, 4 mL). The configuration of the pyran stereocenters is arbitrarily assigned.

LCMS: 99%, RT=2.57 min., (M+H)⁺=385 (method AK).

### Synthesis of ((2R,5R)-5-(aminomethyl)-5-hydroxytetrahydro-2H-pyran-2-yl)((S)-1-(4-fluorophenyl)-3,4-dihydroisoquinolin-2(1H)-yl)methanone (Compound 5047)

Palladium on carbon (10 wt %, containing 50% water, spatula point) was added to a solution of ((2R,5R)-5-(azidomethyl)-5-hydroxytetrahydro-2H-pyran-2-yl)((S)-1-(4-fluorophenyl)-3,4-dihydroisoquinolin-2(1H)-yl)methanone (12.2 mg, 0.030 mmol). The mixture was hydrogenated at room temperature and ambient pressure for 4 hours, diluted with methanol, filtered through a nylon, 0.45 μm filter and the filtrate evaporated under reduced pressure. The residue was purified by acidic preparative MPLC (Linear Gradient: t=0 min 5% A; t=2 min 10% A; t=17 min 50% A; t=18 min 100%; t=23 min 100% A; detection: 210 nm). The product containing fractions were pooled, diluted with saturated aqueous NaHCO₃ (4 mL), and extracted with dichloromethane. The organic layer was passed through a phase separator and evaporated under reduced pressure to give ((2S,5S)-5-(aminomethyl)-5-hydroxytetrahydro-2H-pyran-2-yl)((S)-1-(4-fluorophenyl)-3,4-dihydroisoquinolin-2(1H)-yl)methanone (Compound 5047) after lyophilization from a mixture of acetonitrile and water (1:1, 4 mL). The configuration of the pyran stereocenters is arbitrarily assigned.

LCMS: 99%, RT=1.03 min., (M+H)⁺=385 (method P).

### Example 32

### Synthesis of (trans-5-amino-1,3-dioxan-2-yl)((S)-1-(4-fluorophenyl)-3,4-dihydroisoquinolin-2(1H)-yl)methanone (Compound 5048)

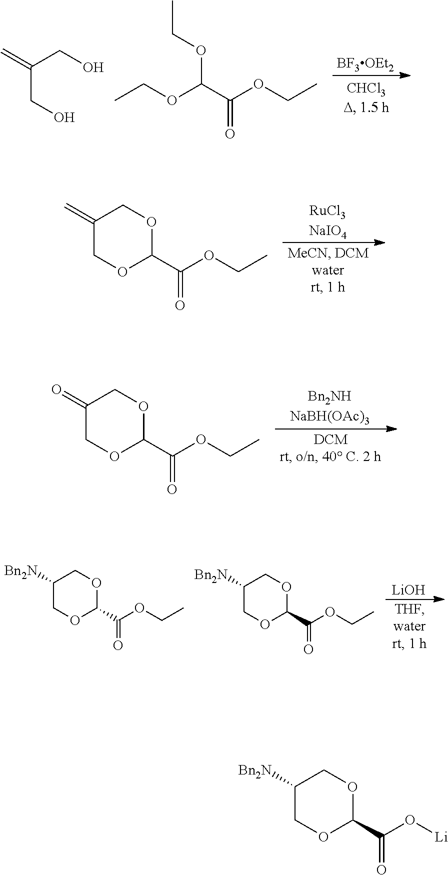

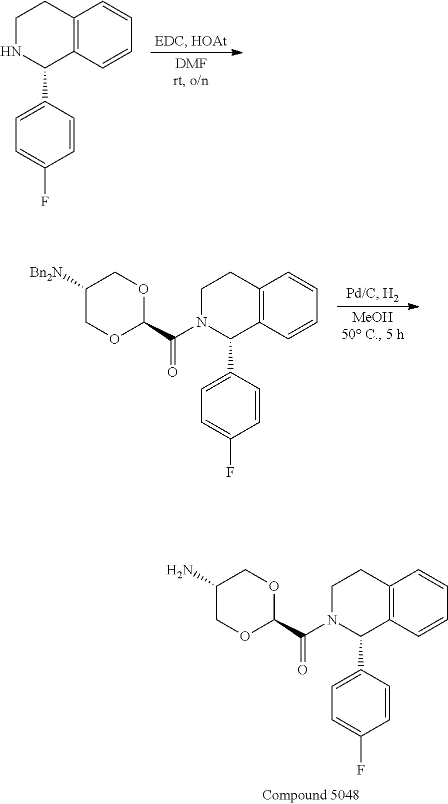

### Synthesis of ethyl 5-methylene-1,3-dioxane-2-carboxylate

In 15 minutes, a suspension of ethyl 2,2-diethoxyacetate (6.58 mL, 36.8 mmol) and 2-methylenepropane-1,3-diol (3 mL, 36.8 mmol) in chloroform (10 mL) was added dropwise to a refluxing solution of boron trifluoride etherate (˜48% BF₃, 9.33 mL, 73.6 mmol) in chloroform (20 ml). After stirring at reflux for 1.5 hour, the reaction mixture was allowed to cool to room temperature, washed successively with water, saturated aqueous Na₂CO₃, and water, dried over Na₂SO₄, and evaporated under reduced pressure. The residue was purified by flash column chromatography (silica, 0 to 50% ethyl acetate in heptane) to give ethyl 5-methylene-1,3-dioxane-2-carboxylate.

GCMS: 96%, RT=2.67 min., (M-CO₂Et)⁺=99 (method A20).

### Synthesis of ethyl 5-oxo-1,3-dioxane-2-carboxylate

Sodium periodate (10.39 g, 48.6 mmol) was added to a mixture of ethyl 5-methylene-1,3-dioxane-2-carboxylate (2.09 g, 12.14 mmol) and ruthenium(III) chloride hydrate (0.109 g, 0.486 mmol) in a mixture of acetonitrile (20 mL), dichloromethane (20 mL), and water (20 mL). The reaction became exothermic and the mixture was cooled with ice/water to ambient temperature. After 1 hour, the reaction mixture was diluted with ethyl acetate, mixed well, and filtered. The residue was washed with ethyl acetate and the combined organic filtrates were washed with brine. The aqueous layer was extracted with ethyl acetate and the combined organic layers were dried over Na₂SO₄ and evaporated under reduced pressure. The residue was purified by flash column chromatography (silica, 0 to 50% ethyl acetate in heptane) to give ethyl 5-oxo-1,3-dioxane-2-carboxylate.

GCMS: RT=2.54 min., (M-CO₂Et)⁺=101 (method A20).

### Synthesis of ethyl cis-5-(dibenzylamino)-1,3-dioxane-2-carboxylate and ethyl (trans)-5-(dibenzylamino)-1,3-dioxane-2-carboxylate

Dibenzylamine (0.222 mL, 1.148 mmol) was added to a solution of ethyl 5-oxo-1,3-dioxane-2-carboxylate (200 mg, 1.148 mmol) in dichloromethane (3 mL). After 15 minutes, sodium triacetoxyborohydride (292 mg, 1.378 mmol) was added and the mixture was stirred overnight. Additional sodium triacetoxyborohydride (243 mg, 1.148 mmol) and acetic acid (2 drops) were added and the reaction mixture was warmed to 40° C. After 2 hours, the reaction mixture was allowed to cool to room temperature and diluted with water and dichloromethane. The layers were separated, and the aqueous phase was diluted with saturated aqueous NaHCO₃ and extracted twice with dichloromethane. The combined organic phase was dried over Na₂SO₄ and evaporated under reduced pressure. The residue was purified by acidic preparative MPLC (Linear Gradient: t=0 min 5% A; t=1 min 5% A; t=2 min 20% A; t=17 min 60% A; t=18 min 100%; t=23 min 100% A; detection: 220/270 nm). The product fractions were combined and lyophilized to give ethyl cis-5-(dibenzylamino)-1,3-dioxane-2-carboxylate as the first eluting isomer and ethyl trans-5-(dibenzylamino)-1,3-dioxane-2-carboxylate as the second eluting isomer after lyophilization of the product containing fractions.

Cis isomer: LCMS: RT=1.76 min., (M+H)⁺=356 (method A).

Trans isomer: LCMS: RT=2.15 min., (M+H)⁺=356 (method A).

### Synthesis of lithium trans-5-(dibenzylamino)-1,3-dioxane-2-carboxylate

Lithium hydroxide monohydrate (5.67 mg, 0.135 mmol) was added to a solution of ethyl trans-5-(dibenzylamino)-1,3-dioxane-2-carboxylate (32 mg, 0.090 mmol) in a mixture of water (2 mL) and tetrahydrofuran (2 mL). After 1 hour, the reaction mixture was concentrated to dryness under reduced pressure to give lithium trans-5-(dibenzylamino)-1,3-dioxane-2-carboxylate which was used as such.

LCMS: RT=1.73 min., (M−Li)⁻=326 (method B).

### Synthesis of (trans-5-(dibenzylamino)-1,3-dioxan-2-yl)((S)-1-(4-fluorophenyl)-3,4-dihydroisoquinolin-2(1H)-yl)methanone

(S)-1-(4-fluorophenyl)-1,2,3,4-tetrahydroisoquinoline (20.46 mg, 0.090 mmol), N-(3-dimethylaminopropyl)-N′-ethylcarbodiimide hydrochloride (20.70 mg, 0.108 mmol), and 1-hydroxy-7-azabenzotriazole (1.225 mg, 9.00 μmol) were added to a solution of lithium trans-5-(dibenzylamino)-1,3-dioxane-2-carboxylate (0.090 mmol) in N,N-dimethylformamide (dry, 3 mL). After stirring the reaction mixture overnight, aqueous HCl (1 M, few drops) and N-(3-dimethylaminopropyl)-N′-ethylcarbodiimide hydrochloride (8.63 mg, 0.045 mmol) were added and stirring was continued for 1 hour. The mixture was diluted with water and extracted 3 times with dichloromethane. The combined organic phase was dried over Na₂SO₄ and evaporated under reduced pressure. The residue was purified by basic preparative MPLC (Linear Gradient: t=0 min 10% A; t=1 min 10% A; t=2 min 40% A; t=17 min 80% A; t=18 min 100% A; t=23 min 100% A; detection: 210/220/270 nm) to give (trans-5-(dibenzylamino)-1,3-dioxan-2-yl)((S)-1-(4-fluorophenyl)-3,4-dihydroisoquinolin-2(1H)-yl)methanone after concentration and lyophilization of the product containing fractions.

LCMS: 100%, RT=2.48 min., (M+H)⁺=537 (method B).

### Synthesis of (trans-5-amino-1,3-dioxan-2-yl)((S)-1-(4-fluorophenyl)-3,4-dihydroisoquinolin-2(1H)-yl)methanone (Compound 5048)

A solution of (trans-5-(dibenzylamino)-1,3-dioxan-2-yl)((S)-1-(4-fluorophenyl)-3,4-dihydroisoquinolin-2(1H)-yl)methanone (27 mg, 0.050 mmol) in methanol (5 mL) was hydrogenated in the presence of palladium (10 wt % on carbon, containing 50% water, 8 mg, 3.76 μmol) at 50° C. and atmospheric hydrogen pressure. After 5 hours, the reaction mixture was filtered and the filtrate was purified by basic preparative MPLC (Linear Gradient: t=0 min 5% A; t=1 min 5% A; t=2 min 20% A; t=17 min 60% A; t=18 min 100% A; t=23 min 100% A; detection: 210/220/270 nm) to give (trans-5-amino-1,3-dioxan-2-yl)((S)-1-(4-fluorophenyl)-3,4-dihydroisoquinolin-2(1)-yl)methanone (Compound 5048) after concentration and lyophilization of the product containing fractions.

LCMS: 100%, RT=1.03 min., (M+H)⁺=357 (method P).

### Example 33

### Synthesis of (cis-5-amino-1,3-dioxan-2-yl)((S)-1-(4-fluorophenyl)-3,4-dihydroisoquinolin-2(1H)-yl)methanone (Compound 5049) and (cis-5-(ethylamino)-1,3-dioxan-2-yl)((S)-1-(4-fluorophenyl)-3,4-dihydroisoquinolin-2(1H)-yl)methanone (Compound 5050)

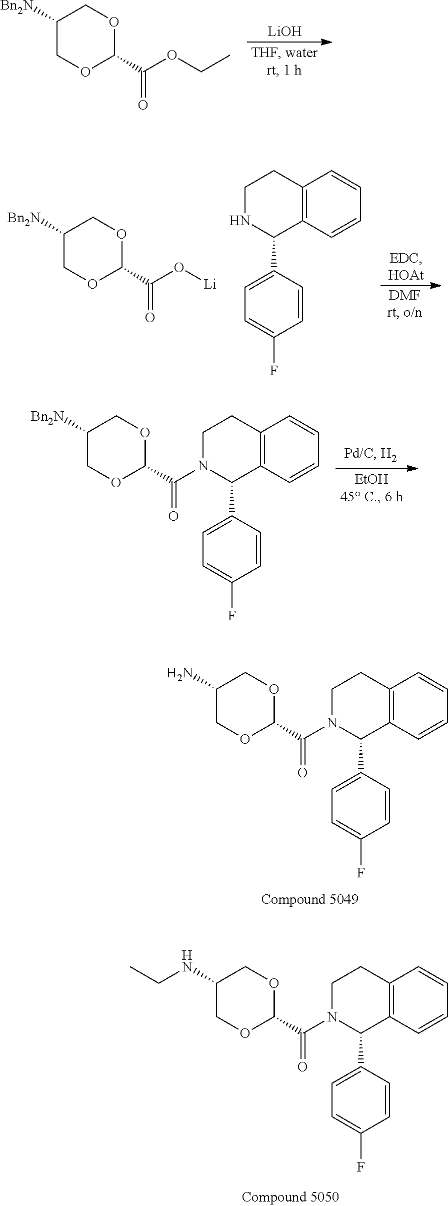

### Synthesis of lithium cis-5-(dibenzylamino)-1,3-dioxane-2-carboxylate

Lithium hydroxide monohydrate (13.22 mg, 0.315 mmol) was added to a solution of ethyl cis-5-(dibenzylamino)-1,3-dioxane-2-carboxylate (56 mg, 0.158 mmol) in a mixture of water (2 mL) and tetrahydrofuran (2 mL). After 1 hour, the reaction mixture was diluted with aqueous HCl (1 M, 0.150 mL, 0.150 mmol) and concentrated to dryness under reduced pressure to give lithium cis-5-(dibenzylamino)-1,3-dioxane-2-carboxylate which was used as such.

LCMS: RT=1.73 min., (M−Li)⁻=326 (method B).

### Synthesis of (cis-5-(dibenzylamino)-1,3-dioxan-2-yl)((S)-1-(4-fluorophenyl)-3,4-dihydroisoquinolin-2(1H)-yl)methanone

Starting from (S)-1-(4-fluorophenyl)-1,2,3,4-tetrahydroisoquinoline (36.1 mg, 0.159 mmol) and lithium cis-5-(dibenzylamino)-1,3-dioxane-2-carboxylate (0.159 mmol), (cis-5-(dibenzylamino)-1,3-dioxan-2-yl)((S)-1-(4-fluorophenyl)-3,4-dihydroisoquinolin-2(1H)-yl)methanone was prepared as described for (trans-5-(dibenzylamino)-1,3-dioxan-2-yl)((S)-1-(4-fluorophenyl)-3,4-dihydroisoquinolin-2(1H)-yl)methanone (see Compound 5048).

LCMS: 100%, RT=2.48 min., (M+H)⁺=537 (method B).

### Synthesis of (cis-5-amino-1,3-dioxan-2-yl)((S)-1-(4-fluorophenyl)-3,4-dihydroisoquinolin-2(1H)-yl)methanone (Compound 5049) and (cis-5-(ethylamino)-1,3-dioxan-2-yl)((S)-1-(4-fluorophenyl)-3,4-dihydroisoquinolin-2(1H)-yl)methanone (Compound 5050)

A solution of (cis-5-(dibenzylamino)-1,3-dioxan-2-yl)((S)-1-(4-fluorophenyl)-3,4-dihydroisoquinolin-2(1H)-yl)methanone (24 mg, 0.045 mmol) in ethanol (5 mL) was hydrogenated in the presence of palladium (10 wt % on carbon, containing 50% water, 8 mg, 3.76 μmol) at 45° C. and atmospheric hydrogen pressure. After 5 hours, the reaction mixture was filtered and the filtrate was purified by basic preparative MPLC (Linear Gradient: t=0 min 5% A; t=1 min 5% A; t=2 min 20% A; t=17 min 60% A; t=18 min 100% A; t=23 min 100% A; detection: 210/220/270 nm) to give (cis-5-amino-1,3-dioxan-2-yl)((S)-1-(4-fluorophenyl)-3,4-dihydroisoquinolin-2(1H)-yl)methanone (Compound 5049) and (cis-5-(ethylamino)-1,3-dioxan-2-yl)((S)-1-(4-fluorophenyl)-3,4-dihydroisoquinolin-2(1H)-yl)methanone (Compound 5050) after concentration and lyophilization of the product containing fractions.

Compound 5049: LCMS: 100%, RT=1.03 min., (M+H)⁺=357 (method P).

Compound 5050: LCMS: 97%, RT=1.06 min., (M+H)⁺=385 (method P).

### Example 34

### Synthesis of ((2R,4S,5S)-4-amino-5-hydroxytetrahydro-2H-pyran-2-yl)((S)-1-(4-fluorophenyl)-3,4-dihydroisoquinolin-2(1H)-yl)methanone (Compound 5051)

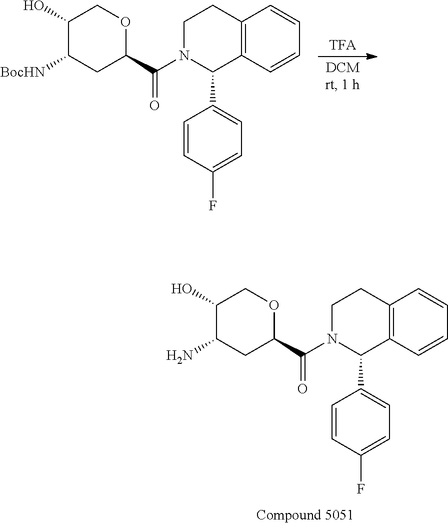 [SMILES: CC(C)(C)OC(=O)N[C@H]1C[C@H](C(=O)N2CCc3ccccc3[C@@H]2c2ccc(F)cc2)OC[C@H]1O ; N[C@H]1C[C@H](C(=O)N2CCc3ccccc3[C@@H]2c2ccc(F)cc2)OC[C@H]1O]

### Synthesis of ((2R,4S,5S)-4-amino-5-hydroxytetrahydro-2H-pyran-2-yl)((S)-1-(4-fluorophenyl)-3,4-dihydroisoquinolin-2(1H)-yl)methanone (Compound 5051)

Starting from tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 53 mg, 0.113 mmol), ((2R,4S,5S)-4-amino-5-hydroxytetrahydro-2H-pyran-2-yl)((S)-1-(4-fluorophenyl)-3,4-dihydroisoquinolin-2(1H)-yl)methanone (Compound 5051) was prepared as described for ((S)-1-(4-fluorophenyl)-3,4-dihydroisoquinolin-2(1H)-yl)((2R,5S)-5-((2-methoxyethyl)amino)tetrahydro-2H-pyran-2-yl)methanone (Compound 5030) without the need for additional purification.

LCMS: 98%, RT=1.03 min., (M+H)⁺=371 (method P). SFC: RT=4.64 min., (M+H)⁺=371 (method F).

### Example 35

### Synthesis of ((2R,4S,5R)-5-amino-4-hydroxytetrahydro-2H-pyran-2-yl)((S)-1-(4-fluorophenyl)-3,4-dihydroisoquinolin-2(1H)-yl)methanone (Compound 5052)

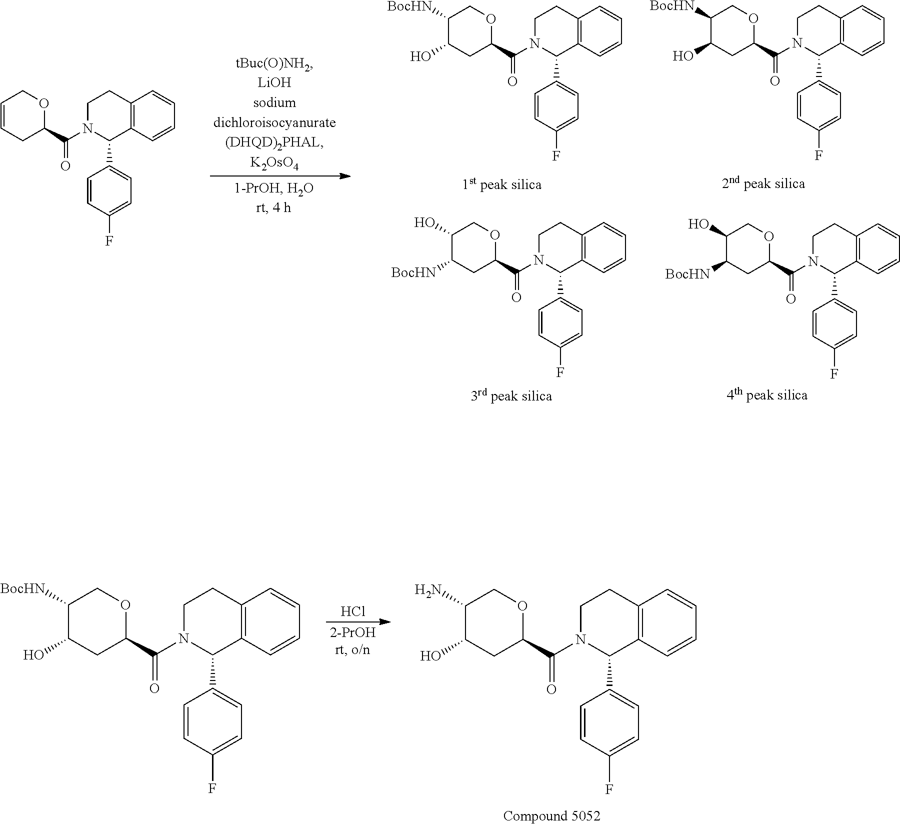

Synthesis of tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate, tert-butyl ((3S,4R,6R)-6-((S)-1-(4-fluorophenyl)-1,2,3,4-tetrahydroisoquinoline-2-carbonyl)-4-hydroxytetrahydro-2H-pyran-3-yl)carbamate, tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate, and tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate.

Starting from ((R)-3,6-dihydro-2H-pyran-2-yl)((S)-1-(4-fluorophenyl)-3,4-dihydroisoquinolin-2(1H)-yl)methanone (2.45 g, 7.26 mmol), the crude products were prepared as described for tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (see Compound 5053). The crude products were purified by flash column chromatography (silica, 0 to 70% ethyl acetate in heptane) to give tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate as the first eluting isomer on silica after crystallization from 2-propanol and further purification of mixed fractions by chiral preparative SFC (method S), tert-butyl ((3S,4R,6R)-6-((S)-1-(4-fluorophenyl)-1,2,3,4-tetrahydroisoquinoline-2-carbonyl)-4-hydroxytetrahydro-2H-pyran-3-yl)carbamate as the second eluting isomer on silica after further purification by chiral preparative SFC (method BG), tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate as the third eluting isomer on silica, and tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate as the fourth eluting isomer on silica after further purification by chiral preparative SFC (method A1).

First eluting isomer on silica: LCMS: 100%, RT=2.07 min., (M+H)⁺=471 (method A). SFC: RT=3.59 min., (M+H)⁺=471 (method F).

Second eluting isomer on silica: LCMS: 95%, RT=2.06 min., (M+H)⁺=471 (method A). SFC: RT=3.06 min., (M+H)⁺=471 (method F).

Third eluting isomer on silica: LCMS: 100%, RT=2.06 min., (M+H)⁺=471 (method A). SFC: RT=3.32 min., (M+H)⁺=471 (method F).

Fourth eluting isomer on silica: LCMS: 93%, RT=2.05 min., (M-Boc+H)⁺=371 (method A). SFC: RT=3.07 min., (M-Boc+H)⁺=371 (method F).

### Synthesis of ((2R,4S,5R)-5-amino-4-hydroxytetrahydro-2H-pyran-2-yl)((S)-1-(4-fluorophenyl)-3,4-dihydroisoquinolin-2(1H)-yl)methanone (Compound 5052)

Starting from tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (30 mg, 0.064 mmol), ((2R,4S,5R)-5-amino-4-hydroxytetrahydro-2H-pyran-2-yl)((S)-1-(4-fluorophenyl)-3,4-dihydroisoquinolin-2(1H)-yl)methanone (Compound 5052) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.00 min., (M+H)⁺=371 (method P). SFC: RT=4.47 min., (M+H)⁺=371 (method F).

### Example 36

### Synthesis of ((2S,4S,5R)-5-amino-4-hydroxytetrahydro-2H-pyran-2-yl)((S)-1-(4-fluorophenyl)-3,4-dihydroisoquinolin-2(1H)-yl)methanone (Compound 5053)

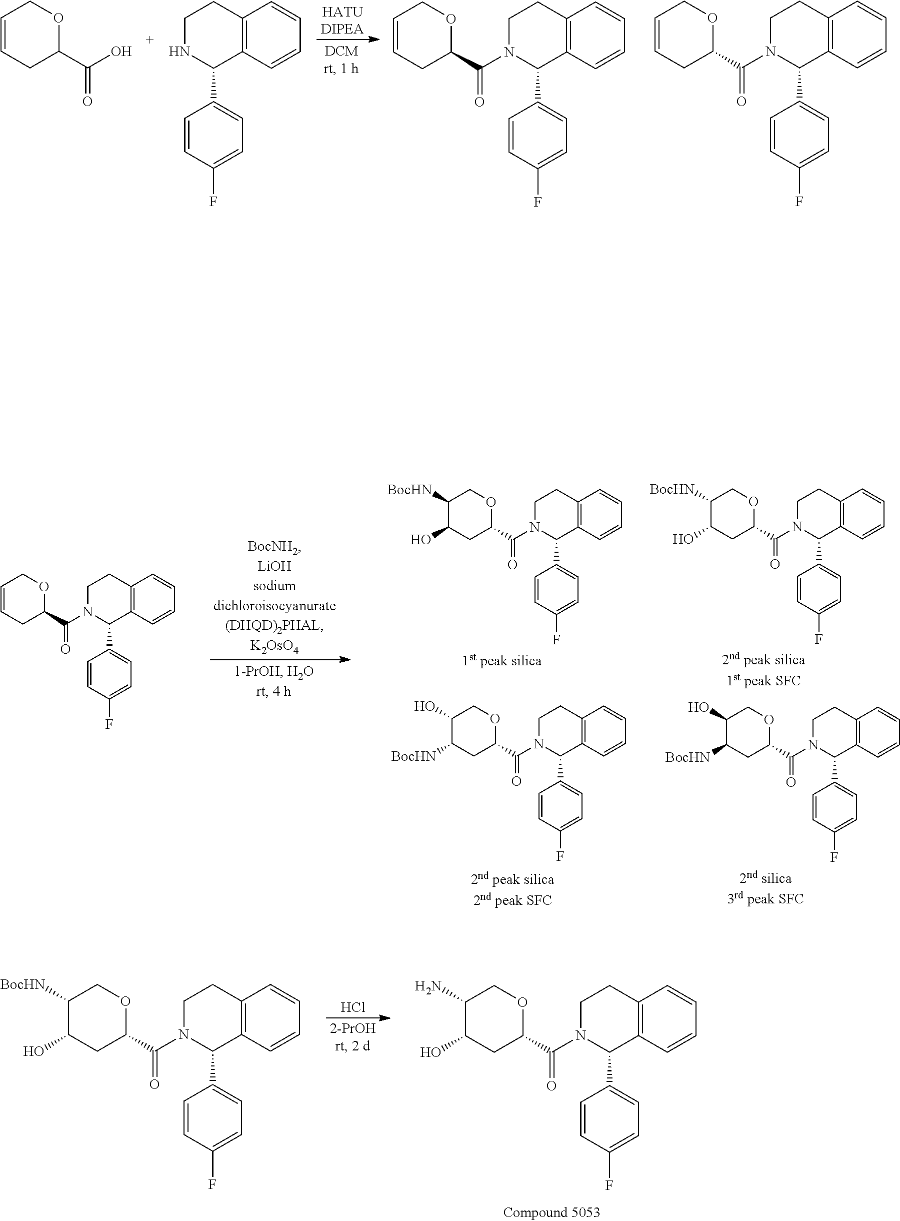

### Synthesis of ((R)-3,6-dihydro-2H-pyran-2-yl)((S)-1-(4-fluorophenyl)-3,4-dihydroisoquinolin-2(1H)-yl)methanone and ((S)-3,6-dihydro-2H-pyran-2-yl)((S)-1-(4-fluorophenyl)-3,4-dihydroisoquinolin-2(1H)-yl)methanone

1-[Bis(dimethylamino)methylene]-1H-1,2,3-triazolo[4,5-b]pyridinium 3-oxide hexafluorophosphate (32.6 g, 86 mmol) and N,N-diisopropylethylamine (20.45 mL, 117 mmol) were added to a solution of 3,6-dihydro-2H-pyran-2-carboxylic acid (10 g, 78 mmol) in dichloromethane (150 mL). After 10 minutes, (S)-1-(4-fluorophenyl)-1,2,3,4-tetrahydroisoquinoline (19.51 g, 86 mmol) was added and stirring was continued for 1 hour. The reaction mixture was diluted with dichloromethane (120 mL), washed with saturated aqueous NaHCO₃ (2×50 mL) and aqueous HCl (1 M, 2×50 mL), dried over Na₂SO₄, and evaporated under reduced pressure. The residue was purified by flash column chromatography (silica, 0 to 50% ethyl acetate in heptane) to give ((R)-3,6-dihydro-2H-pyran-2-yl)((S)-1-(4-fluorophenyl)-3,4-dihydroisoquinolin-2(1H)-yl)methanone as the first eluting isomer and ((S)-3,6-dihydro-2H-pyran-2-yl)((S)-1-(4-fluorophenyl)-3,4-dihydroisoquinolin-2(1H)-yl)methanone as the second eluting isomer.

First eluting isomer: LCMS: 87%, RT=2.12 min., (M+H)⁺=338 (method A). SFC: 100%, RT=3.18 min., (M+H)⁺=338 (method F).

Second eluting isomer: LCMS: 95%, RT=2.12 min., (M+H)⁺=338 (method A). SFC: 100%, RT=2.72 min., (M+H)⁺=338 (method F).

Synthesis of tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate, tert-butyl ((3R,4S,6S)-6-((S)-1-(4-fluorophenyl)-1,2,3,4-tetrahydroisoquinoline-2-carbonyl)-4-hydroxytetrahydro-2H-pyran-3-yl)carbamate, tert-butyl ((2S,4S,5S)-2-((S)-1-(4-fluorophenyl)-1,2,3,4-tetrahydroisoquinoline-2-carbonyl)-5-hydroxytetrahydro-2H-pyran-4-yl)carbamate, and tert-butyl ((2S,4R,5R)-2-((S)-1-(4-fluorophenyl)-1,2,3,4-tetrahydroisoquinoline-2-carbonyl)-5-hydroxytetrahydro-2H-pyran-4-yl)carbamate.

Sodium dichloroisocyanurate (1.29 g, 5.84 mmol) was added to stirred mixture of tert-butyl carbamate (684 mg, 5.84 mmol) in 1-propanol (26 mL) and aqueous LiOH (0.5 M, 11.3 mL, 5.65 mmol). After 15 minutes, a fine suspension was formed and a freshly prepared solution of hydroquinidine 1,4-phthalazinediyl diether ((DHQD)₂PHAL, 76 mg, 0.097 mmol) and ((S)-3,6-dihydro-2H-pyran-2-yl)((S)-1-(4-fluorophenyl)-3,4-dihydroisoquinolin-2(1H)-yl)methanone (657 mg, 1.95 mmol) in 1-propanol (13 mL) was added slowly. The reaction mixture was stirred for another 2 minutes, after which a solution of potassium osmate(VI) dihydrate (28.7 mg, 0.078 mmol) in water (8.6 mL) was added dropwise. After 3.5 hours, saturated aqueous sodium metabisulfite was added (10 mL) and the mixture was stirred for another 15 minutes. The mixture was filtered through a glass filter (por-4) and washed with 1-propanol. The volatiles of the filtrate were removed under reduced pressure and the aqueous residue was extracted with dichloromethane (2×20 mL). The combined organic phase was washed with brine, dried over Na₂SO₄, and evaporated under reduced pressure. The residue was purified by flash column chromatography (silica, 0 to 100% ethyl acetate in heptane) to give tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate as the first eluting isomer on silica after further purification by flash column chromatography (silica, 0 to 10% 2-propanol in heptane). The second eluting fraction on silica was further purified by chiral preparative SFC (method BH) to give tert-butyl ((3R,4S,6S)-6-((S)-1-(4-fluorophenyl)-1,2,3,4-tetrahydroisoquinoline-2-carbonyl)-4-hydroxytetrahydro-2H-pyran-3-yl)carbamate as the first eluting isomer on SFC, tert-butyl ((2S,4S,5S)-2-((S)-1-(4-fluorophenyl)-1,2,3,4-tetrahydroisoquinoline-2-carbonyl)-5-hydroxytetrahydro-2H-pyran-4-yl)carbamate as the second eluting isomer on SFC, and tert-butyl ((2S,4R,5R)-2-((S)-1-(4-fluorophenyl)-1,2,3,4-tetrahydroisoquinoline-2-carbonyl)-5-hydroxytetrahydro-2H-pyran-4-yl)carbamate as the third eluting isomer on SFC.

First eluting isomer on silica: LCMS: 85%, RT=2.07 min., (M+H)⁺=471 (method A). SFC: RT=2.31 min., (M+H)⁺=471 (method AU).

Second eluting fraction on silica; first eluting isomer on SFC: SFC: RT=1.97 min., (M+H)⁺=471 (method AU).

Second eluting fraction on silica; second eluting isomer on SFC: SFC: RT=2.10 min., (M-Boc+H)⁺=371 (method AU).

Second eluting fraction on silica; third eluting isomer on SFC: SFC: RT=2.31 min., (M+H)⁺=471 (method AU).

### Synthesis of ((2S,4S,5R)-5-amino-4-hydroxytetrahydro-2H-pyran-2-yl)((S)-1-(4-fluorophenyl)-3,4-dihydroisoquinolin-2(1H)-yl)methanone (Compound 5053)

Starting from tert-butyl ((3R,4S,6S)-6-((S)-1-(4-fluorophenyl)-1,2,3,4-tetrahydroisoquinoline-2-carbonyl)-4-hydroxytetrahydro-2H-pyran-3-yl)carbamate (16 mg, 0.034 mmol), ((2S,4S,5R)-5-amino-4-hydroxytetrahydro-2H-pyran-2-yl)((S)-1-(4-fluorophenyl)-3,4-dihydroisoquinolin-2(1H)-yl)methanone (Compound 5053) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.01 min., (M+H)⁺=371 (method P). SFC: RT=3.94 min., (M+H)⁺=371 (method W).

### Example 37

### Synthesis of ((2S,4R,5R)-4-amino-5-hydroxytetrahydro-2H-pyran-2-yl)((S)-1-(4-fluorophenyl)-3,4-dihydroisoquinolin-2(1H)-yl)methanone (Compound 5054)

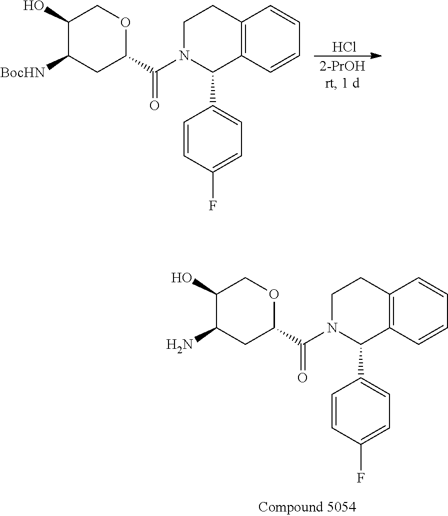 [SMILES: CC(C)(C)OC(=O)N[C@@H]1C[C@@H](C(=O)N2CCc3ccccc3[C@@H]2c2ccc(F)cc2)OC[C@@H]1O ; Cl ; N[C@@H]1C[C@@H](C(=O)N2CCc3ccccc3[C@@H]2c2ccc(F)cc2)OC[C@@H]1O]

### Synthesis of ((2S,4R,5R)-4-amino-5-hydroxytetrahydro-2H-pyran-2-yl)((S)-1-(4-fluorophenyl)-3,4-dihydroisoquinolin-2(1H)-yl)methanone (Compound 5054)

Starting from tert-butyl ((2S,4R,5R)-2-((S)-1-(4-fluorophenyl)-1,2,3,4-tetrahydroisoquinoline-2-carbonyl)-5-hydroxytetrahydro-2H-pyran-4-yl)carbamate (See Compound 5053, 33 mg, 0.070 mmol), ((2S,4R,5R)-4-amino-5-hydroxytetrahydro-2H-pyran-2-yl)((S)-1-(4-fluorophenyl)-3,4-dihydroisoquinolin-2(1H)-yl)methanone (Compound 5054) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.02 min., (M+H)⁺=371 (method P). SFC: RT=4.48 min., (M+H)⁺=371 (method AD).

### Example 38

### Synthesis of ((2R,4S,5R)-4-amino-5-hydroxytetrahydro-2H-pyran-2-yl)((S)-1-(4-fluorophenyl)-3,4-dihydroisoquinolin-2(1H)-yl)methanone (Compound 5055)

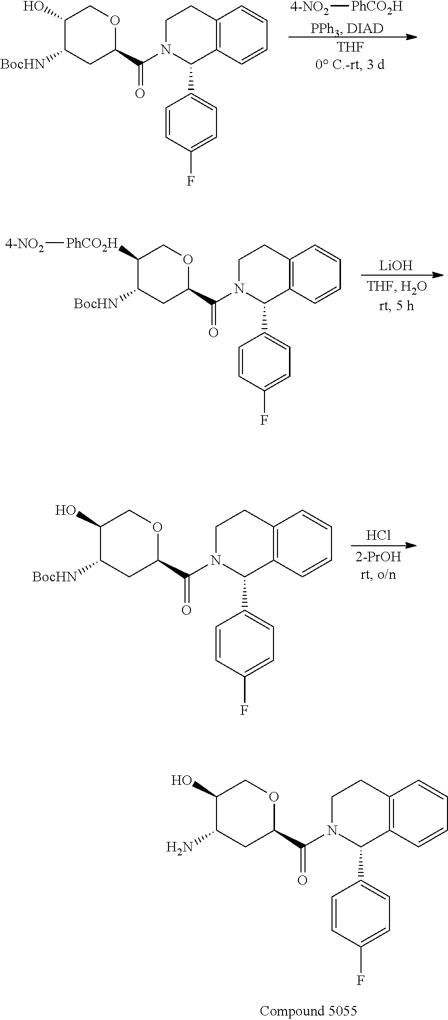

### Synthesis of (3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate

Starting from tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (See Compound 5052, 50 mg, 0.106 mmol), (3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate was prepared as described for (2S,4S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl 4-nitrobenzoate (see Compound 5056) with recharging of triphenylphosphine and diisopropyl azodicarboxylate after 2 days and used directly in the next step.

LCMS: 93%, RT=2.30 min., (M+Na)⁺=642 (method A).

### Synthesis of tert-butyl ((2R,4S,5R)-2-((S)-1-(4-fluorophenyl)-1,2,3,4-tetrahydroisoquinoline-2-carbonyl)-5-hydroxytetrahydro-2H-pyran-4-yl)carbamate

Starting from (3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate (67 mg crude, 0.106 mmol), tert-butyl ((2R,4S,5R)-2-((S)-1-(4-fluorophenyl)-1,2,3,4-tetrahydroisoquinoline-2-carbonyl)-5-hydroxytetrahydro-2H-pyran-4-yl)carbamate was prepared as described for tert-butyl ((3S,4S,6S)-6-((S)-1-(4-fluorophenyl)-1,2,3,4-tetrahydroisoquinoline-2-carbonyl)-4-hydroxytetrahydro-2H-pyran-3-yl)carbamate (see Compound 5056).

LCMS: 100%, RT=2.05 min., (M+H)⁺=471 (method A).

### Synthesis of ((2R,4S,5R)-4-amino-5-hydroxytetrahydro-2H-pyran-2-yl)((S)-1-(4-fluorophenyl)-3,4-dihydroisoquinolin-2(1H)-yl)methanone (Compound 5055)

Starting from tert-butyl ((2R,4S,5R)-2-((S)-1-(4-fluorophenyl)-1,2,3,4-tetrahydroisoquinoline-2-carbonyl)-5-hydroxytetrahydro-2H-pyran-4-yl)carbamate (11 mg, 0.023 mmol), ((2R,4S,5R)-4-amino-5-hydroxytetrahydro-2H-pyran-2-yl)((S)-1-(4-fluorophenyl)-3,4-dihydroisoquinolin-2(1H)-yl)methanone (Compound 5055) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.03 min., (M+H)⁺=371 (method P). SFC: RT=3.68 min., (M+H)⁺=371 (method AD).

### Example 39

### Synthesis of ((2S,4S,5S)-5-amino-4-hydroxytetrahydro-2H-pyran-2-yl)((S)-1-(4-fluorophenyl)-3,4-dihydroisoquinolin-2(1H)-yl)methanone (Compound 5056)

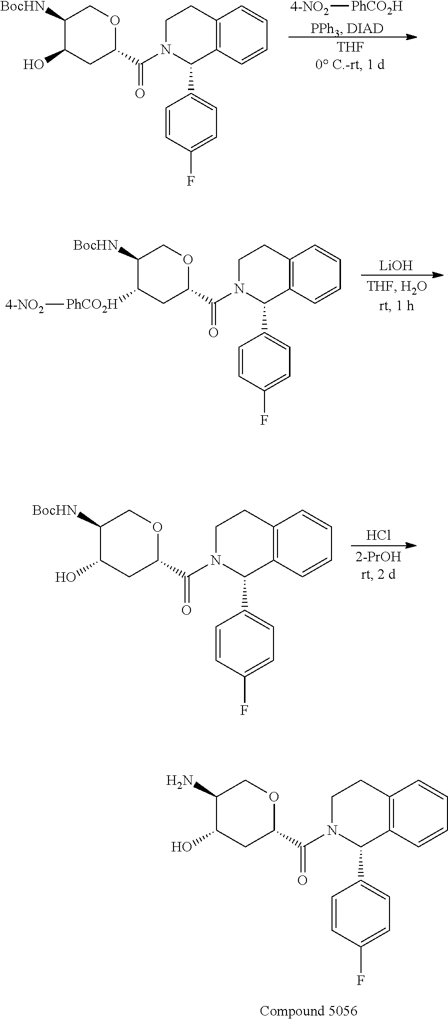

### Synthesis of (2S,4S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl 4-nitrobenzoate

Under an argon atmosphere at 0° C., diisopropyl azodicarboxylate (0.025 mL, 0.128 mmol) was added to a solution of tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (See Compound 5053, 40 mg, 0.085 mmol), triphenylphosphine (33.4 mg, 0.128 mmol) and 4-nitrobenzoic acid (21.3 mg, 0.128 mmol) in dry tetrahydrofuran (0.4 mL). The reaction mixture was allowed to warm to room temperature and stirred for 1 day. The reaction mixture was diluted with ethyl acetate (5 mL) and washed with water (5 mL). The aqueous phase was extracted with ethyl acetate (2×5 mL) and the combined phase was dried over Na₂SO₄ and evaporated under reduced pressure.

The residue was purified by flash column chromatography (silica, 0 to 35% ethyl acetate in heptane) to give (2S,4S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl 4-nitrobenzoate which was used as such.

LCMS: 98%, RT=2.27 min., (M+H)⁺=620 (method A).

### Synthesis of tert-butyl ((3S,4S,6S)-6-((S)-1-(4-fluorophenyl)-1,2,3,4-tetrahydroisoquinoline-2-carbonyl)-4-hydroxytetrahydro-2H-pyran-3-yl)carbamate

A solution of lithium hydroxide monohydrate (2.98 mg, 0.071 mmol) in water (0.5 mL) was added to a solution of (2S,4S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl 4-nitrobenzoate (40 mg, 0.065 mmol) in tetrahydrofuran (0.5 mL). After stirring for 1 hour, the mixture was diluted with dichloromethane (10 mL) and saturated aqueous NaHCO₃ (10 mL). The aqueous layer was extracted with dichloromethane (2×10 mL) and the combined phase was dried over Na₂SO₄ and evaporated under reduced pressure. The residue was purified by flash column chromatography (silica, 0 to 70% ethyl acetate in heptane) to give tert-butyl ((3S,4S,6S)-6-((S)-1-(4-fluorophenyl)-1,2,3,4-tetrahydroisoquinoline-2-carbonyl)-4-hydroxytetrahydro-2H-pyran-3-yl)carbamate.

LCMS: 100%, RT=2.03 min., (M+H)⁺=471 (method A).

### Synthesis of ((2S,4S,5S)-5-amino-4-hydroxytetrahydro-2H-pyran-2-yl)((S)-1-(4-fluorophenyl)-3,4-dihydroisoquinolin-2(1H)-yl)methanone (Compound 5056)

Starting from tert-butyl ((3S,4S,6S)-6-((S)-1-(4-fluorophenyl)-1,2,3,4-tetrahydroisoquinoline-2-carbonyl)-4-hydroxytetrahydro-2H-pyran-3-yl)carbamate (15 mg, 0.032 mmol), ((2S,4S,5S)-5-amino-4-hydroxytetrahydro-2H-pyran-2-yl)((S)-1-(4-fluorophenyl)-3,4-dihydroisoquinolin-2(1H)-yl)methanone (Compound 5056) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 98%, RT=1.01 min., (M+H)⁺=371 (method P). SFC: RT=4.55 min., (M+H)⁺=371 (method F).

### Example 40

### Synthesis of ((2R,4R,5R)-5-amino-4-hydroxytetrahydro-2H-pyran-2-yl)((S)-1-(4-fluorophenyl)-3,4-dihydroisoquinolin-2(1H)-yl)methanone (Compound 5057)

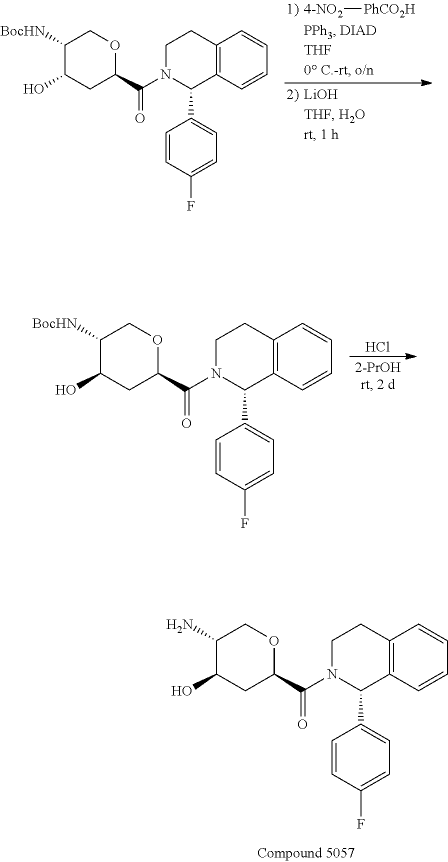

### Synthesis of tert-butyl ((3R,4R,6R)-6-((S)-1-(4-fluorophenyl)-1,2,3,4-tetrahydroisoquinoline-2-carbonyl)-4-hydroxytetrahydro-2H-pyran-3-yl)carbamate

Starting from tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (See Compound 5052, 40 mg, 0.085 mmol), tert-butyl ((3R,4R,6R)-6-((S)-1-(4-fluorophenyl)-1,2,3,4-tetrahydroisoquinoline-2-carbonyl)-4-hydroxytetrahydro-2H-pyran-3-yl)carbamate was prepared as described for (2S,4S,5S)-5-((tert-butoxycarbonyl)amino)-2-((S)-1-(4-fluorophenyl)-1,2,3,4-tetrahydroisoquinoline-2-carbonyl)tetrahydro-2H-pyran-4-yl 4-nitrobenzoate (see Compound 5056) and tert-butyl ((3S,4S,6S)-6-((S)-1-(4-fluorophenyl)-1,2,3,4-tetrahydroisoquinoline-2-carbonyl)-4-hydroxytetrahydro-2H-pyran-3-yl)carbamate (see Compound 5056). LCMS: 100%, RT=2.03 min., (M+H)⁺=471 (method A).

### Synthesis of ((2R,4R,5R)-5-amino-4-hydroxytetrahydro-2H-pyran-2-yl)((S)-1-(4-fluorophenyl)-3,4-dihydroisoquinolin-2(1H)-yl)methanone (Compound 5057)

Starting from tert-butyl ((3R,4R,6R)-6-((S)-1-(4-fluorophenyl)-1,2,3,4-tetrahydroisoquinoline-2-carbonyl)-4-hydroxytetrahydro-2H-pyran-3-yl)carbamate (29 mg, 0.062 mmol), ((2R,4R,5R)-5-amino-4-hydroxytetrahydro-2H-pyran-2-yl)((S)-1-(4-fluorophenyl)-3,4-dihydroisoquinolin-2(1H)-yl)methanone (Compound 5057) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=2.51 min., (M+H)⁺=371 (method AK). SFC: RT=5.31 min., (M+H)⁺=371 (method F).

### Example 41

### Synthesis of ((2S,4S,5S)-4-amino-5-hydroxytetrahydro-2H-pyran-2-yl)((S)-1-(4-fluorophenyl)-3,4-dihydroisoquinolin-2(1H)-yl)methanone (Compound 5058)

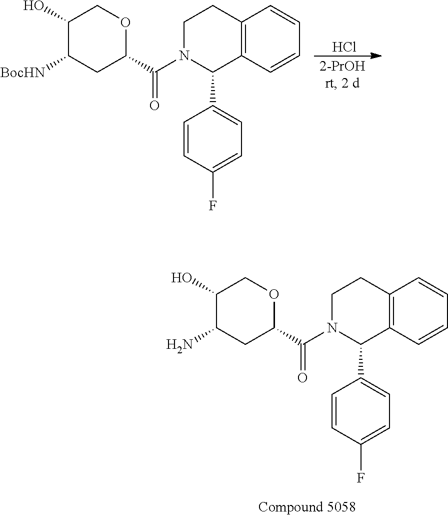 [SMILES: CC(C)(C)OC(=O)N[C@H]1C[C@@H](C(=O)N2CCc3ccccc3[C@@H]2c2ccc(F)cc2)OC[C@H]1O ; Cl ; N[C@H]1C[C@@H](C(=O)N2CCc3ccccc3[C@@H]2c2ccc(F)cc2)OC[C@H]1O]

### Synthesis of ((2S,4S,5S)-4-amino-5-hydroxytetrahydro-2H-pyran-2-yl)((S)-1-(4-fluorophenyl)-3,4-dihydroisoquinolin-2(1H)-yl)methanone (Compound 5058)

Starting from tert-butyl ((2S,4S,5S)-2-((S)-1-(4-fluorophenyl)-1,2,3,4-tetrahydroisoquinoline-2-carbonyl)-5-hydroxytetrahydro-2H-pyran-4-yl)carbamate (See Compound 5053, 35 mg, 0.074 mmol), ((2S,4S,5S)-4-amino-5-hydroxytetrahydro-2H-pyran-2-yl)((S)-1-(4-fluorophenyl)-3,4-dihydroisoquinolin-2(1H)-yl)methanone (Compound 5058) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043). LCMS: 99%, RT=1.01 min., (M+H)⁺=371 (method P). SFC: RT=4.00 min., (M+H)⁺=371 (method AD).

### Example 42

### Synthesis of ((2S,4S,5S)-5-amino-4-fluorotetrahydro-2H-pyran-2-yl)((S)-1-(4-fluorophenyl)-3,4-dihydroisoquinolin-2(1H)-yl)methanone (Compound 5059)

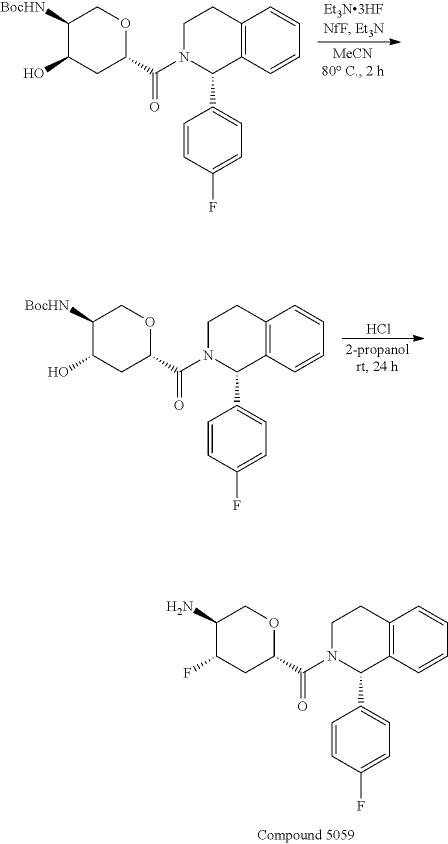 [SMILES: CC(C)(C)OC(=O)N[C@H]1CO[C@H](C(=O)N2CCc3ccccc3[C@@H]2c2ccc(F)cc2)C[C@H]1O ; CC(C)(C)OC(=O)N[C@H]1CO[C@H](C(=O)N2CCc3ccccc3[C@@H]2c2ccc(F)cc2)C[C@@H]1O ; Cl ; N[C@H]1CO[C@H](C(=O)N2CCc3ccccc3[C@@H]2c2ccc(F)cc2)C[C@@H]1F]

### Synthesis of tert-butyl ((3S,4S,6S)-4-fluoro-6-((S)-1-(4-fluorophenyl)-1,2,3,4-tetrahydroisoquinoline-2-carbonyl)tetrahydro-2H-pyran-3-yl)carbamate

Starting from tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (see Compound 5053, 76 mg, 0.162 mmol), tert-butyl ((3S,4S,6S)-4-fluoro-6-((S)-1-(4-fluorophenyl)-1,2,3,4-tetrahydroisoquinoline-2-carbonyl)tetrahydro-2H-pyran-3-yl)carbamate was prepared as described for tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate (see Compound 5069, at 80° C.).

LCMS: 100%, RT=2.15 min., (M+H)⁺=473 (method A).

### Synthesis of ((2S,4S,5S)-5-amino-4-fluorotetrahydro-2H-pyran-2-yl)((S)-1-(4-fluorophenyl)-3,4-dihydroisoquinolin-2(1H)-yl)methanone (Compound 5059)

Starting from tert-butyl ((3S,4S,6S)-4-fluoro-6-((S)-1-(4-fluorophenyl)-1,2,3,4-tetrahydroisoquinoline-2-carbonyl)tetrahydro-2H-pyran-3-yl)carbamate (34 mg, 0.072 mmol), ((2S,4S,5S)-5-amino-4-fluorotetrahydro-2H-pyran-2-yl)((S)-1-(4-fluorophenyl)-3,4-dihydroisoquinolin-2(1H)-yl)methanone (Compound 5059) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043) and purified by preparative chiral SFC (method AN).

LCMS: 100%, RT=1.05 min., (M+H)⁺=373 (method P). SFC: RT=3.56 min., (M+H)⁺=373 (method AD).

### Example 43

### Synthesis of ((2S,5S)-5-amino-4,4-difluorotetrahydro-2H-pyran-2-yl)((S)-1-(4-fluorophenyl)-3,4-dihydroisoquinolin-2(1H)-yl)methanone (Compound 5060)

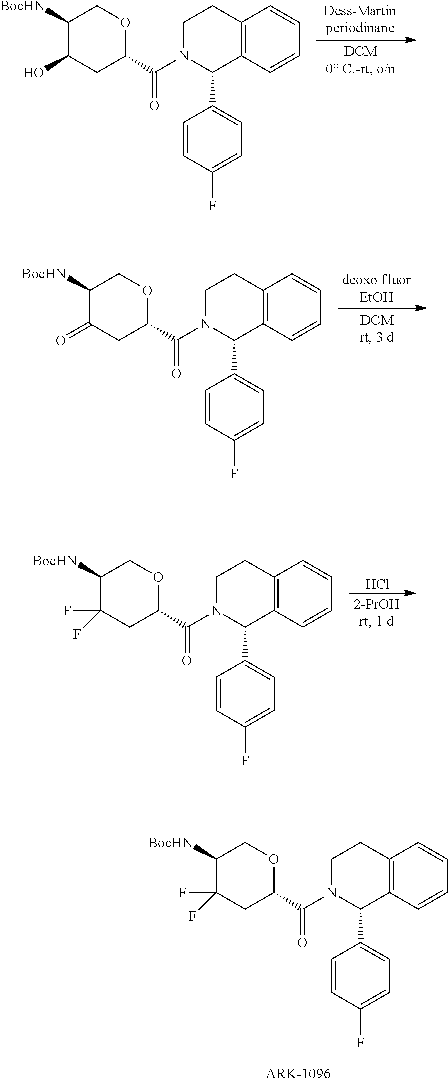 [SMILES: CC(C)(C)OC(=O)N[C@H]1CO[C@H](C(=O)N2CCc3ccccc3[C@@H]2c2ccc(F)cc2)C[C@H]1O ; CC(C)(C)OC(=O)N[C@H]1CO[C@H](C(=O)N2CCc3ccccc3[C@@H]2c2ccc(F)cc2)CC1=O ; CC(C)(C)OC(=O)N[C@H]1CO[C@H](C(=O)N2CCc3ccccc3[C@@H]2c2ccc(F)cc2)CC1(F)F ; Cl ; CC(C)(C)OC(=O)N[C@H]1CO[C@H](C(=O)N2CCc3ccccc3[C@@H]2c2ccc(F)cc2)CC1(F)F]

### Synthesis of tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate

At 0° C., 1,1,1-tris(acetyloxy)-1,1-dihydro-1,2-benziodoxol-3-(1H)-one (Dess-Martin periodinane, 49.6 mg, 0.117 mmol) was added to a solution of tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (see Compound 5053, 50 mg, 0.106 mmol) in dichloromethane (1 mL). The reaction mixture was allowed to warm to room temperature and stirred overnight. The reaction mixture was quenched with a mixture of saturated aqueous Na₂S₂O₃ and saturated aqueous NaHCO₃ (1:1, 2 mL) and stirred for 1 hour. The layers were separated using a phase-separator and the organic filtrate was evaporated under reduced pressure. The residue was purified by flash column chromatography (silica, 0 to 35% ethyl acetate in heptane) to give tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate.

LCMS: 82%, RT=2.15 min., (M+Na)⁺=491 and 18% as hydrate: RT=2.03 min., (M+Na)⁺=509 (method A).

### Synthesis of tert-butyl ((3S,6S)-4,4-difluoro-6-((S)-1-(4-fluorophenyl)-1,2,3,4-tetrahydroisoquinoline-2-carbonyl)tetrahydro-2H-pyran-3-yl)carbamate

At 0° C., a solution of tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate (42 mg, 0.090 mmol) in dichloromethane (0.5 mL) followed by ethanol (0.523 μL, 8.96 μmol) were added to a solution of [bis(2-methoxyethyl)amino]sulfur trifluoride (2.6 M in toluene, 0.072 mL, 0.188 mmol) in dichloromethane (0.20 mL). After the addition a solution of tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate (42 mg, 0.090 mmol) in dichloromethane (0.5 ml) was added, followed by ethanol (0.523 μl, 8.96 μmol). The reaction mixture was allowed to warm to room temperature and stirred for 3 days. The reaction mixture was diluted with dichloromethane (1 mL), cooled in an ice-bath and quenched with saturated aqueous NaHCO₃ (2 mL). The layers were separated using a phase-separator and the organic filtrate was evaporated under reduced pressure. The residue was purified by flash column chromatography (silica, 0 to 30% ethyl acetate in heptane) to give tert-butyl ((3S,6S)-4,4-difluoro-6-((S)-1-(4-fluorophenyl)-1,2,3,4-tetrahydroisoquinoline-2-carbonyl)tetrahydro-2H-pyran-3-yl)carbamate.

LCMS: 100%, RT=2.20 min., (M+Na)⁺=513 (method A).

### Synthesis of ((2S,5S)-5-amino-4,4-difluorotetrahydro-2H-pyran-2-yl)((S)-1-(4-fluorophenyl)-3,4-dihydroisoquinolin-2(1H)-yl)methanone (Compound 5060)

Starting from tert-butyl ((3S,6S)-4,4-difluoro-6-((S)-1-(4-fluorophenyl)-1,2,3,4-tetrahydroisoquinoline-2-carbonyl)tetrahydro-2H-pyran-3-yl)carbamate (23 mg, 0.047 mmol), ((2S,5S)-5-amino-4,4-difluorotetrahydro-2H-pyran-2-yl)((S)-1-(4-fluorophenyl)-3,4-dihydroisoquinolin-2(1H)-yl)methanone (Compound 5060) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.09 min., (M+H)⁺=391 (method P). SFC: RT=4.64 min., (M+H)⁺=371 (method F).

### Example 44

### Synthesis of ((2R,4R,5R)-5-amino-4-fluorotetrahydro-2H-pyran-2-yl)((S)-1-(4-fluorophenyl)-3,4-dihydroisoquinolin-2(1H)-yl)methanone (Compound 5061)

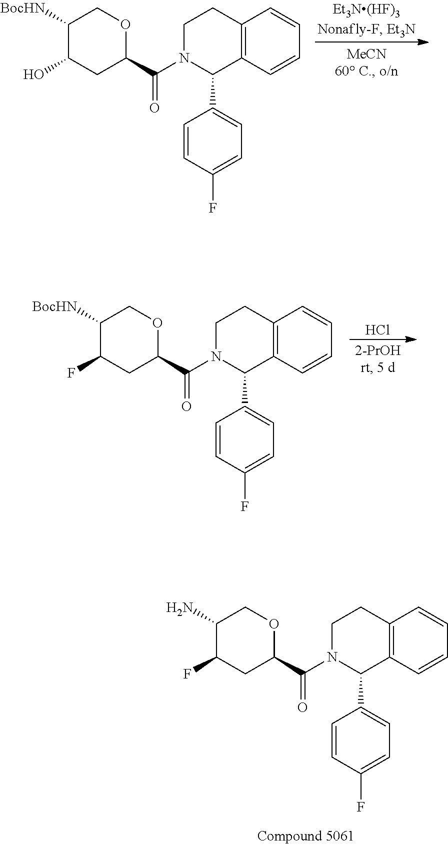 [SMILES: CC(C)(C)OC(=O)N[C@@H]1CO[C@@H](C(=O)N2CCc3ccccc3[C@@H]2c2ccc(F)cc2)C[C@@H]1O ; CC(C)(C)OC(=O)N[C@@H]1CO[C@@H](C(=O)N2CCc3ccccc3[C@@H]2c2ccc(F)cc2)C[C@H]1F ; Cl ; N[C@@H]1CO[C@@H](C(=O)N2CCc3ccccc3[C@@H]2c2ccc(F)cc2)C[C@H]1F]

### Synthesis of tert-butyl ((3R,4R,6R)-4-fluoro-6-((S)-1-(4-fluorophenyl)-1,2,3,4-tetrahydroisoquinoline-2-carbonyl)tetrahydro-2H-pyran-3-yl)carbamate

Starting from tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (See Compound 5052, 100 mg, 0.213 mmol), tert-butyl ((3R,4R,6R)-4-fluoro-6-((S)-1-(4-fluorophenyl)-1,2,3,4-tetrahydroisoquinoline-2-carbonyl)tetrahydro-2H-pyran-3-yl)carbamate was prepared as described for tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate (see Compound 5069).

LCMS: 99%, RT=2.16 min., (M+H)⁺=473 (method A).

### Synthesis of ((2R,4R,5R)-5-amino-4-fluorotetrahydro-2H-pyran-2-yl)((S)-1-(4-fluorophenyl)-3,4-dihydroisoquinolin-2(1H)-yl)methanone (Compound 5061)

Starting from tert-butyl ((3R,4R,6R)-4-fluoro-6-((S)-1-(4-fluorophenyl)-1,2,3,4-tetrahydroisoquinoline-2-carbonyl)tetrahydro-2H-pyran-3-yl)carbamate (86 mg, 0.182 mmol), ((2R,4R,5R)-5-amino-4-fluorotetrahydro-2H-pyran-2-yl)((S)-1-(4-fluorophenyl)-3,4-dihydroisoquinolin-2(1H)-yl)methanone (Compound 5061) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043) and purified by flash column chromatography (silica, 0 to 2% (7 M ammonia in methanol) in dichloromethane).

LCMS: 99%, RT=1.04 min., (M+H)⁺=373 (method P). SFC: RT=3.37 min., (M+H)⁺=373 (method AD).

### Example 45

### Synthesis of ((2R,4S,5S)-4-amino-5-methoxytetrahydro-2H-pyran-2-yl)((S)-1-(4-fluorophenyl)-3,4-dihydroisoquinolin-2(1H)-yl)methanone (Compound 5062)

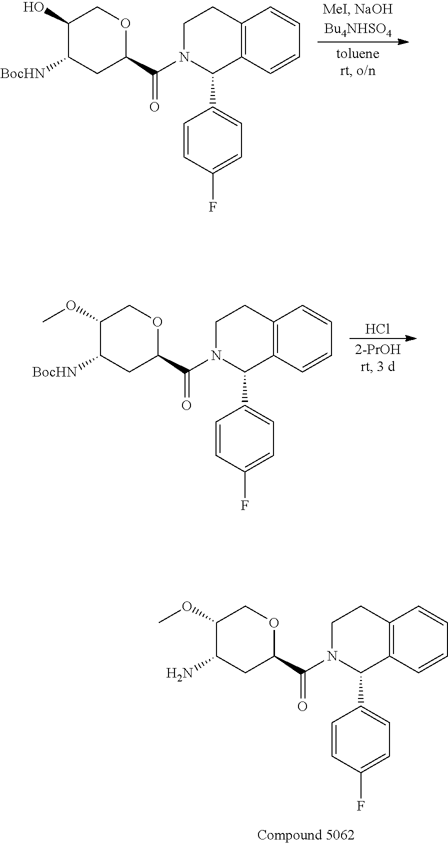 [SMILES: CC(C)(C)OC(=O)N[C@H]1C[C@H](C(=O)N2CCc3ccccc3[C@@H]2c2ccc(F)cc2)OC[C@@H]1O ; CO[C@@H]1CO[C@@H](C(=O)N2CCc3ccccc3[C@@H]2c2ccc(F)cc2)C[C@@H]1NC(=O)OC(C)(C)C ; Cl ; CO[C@@H]1CO[C@@H](C(=O)N2CCc3ccccc3[C@@H]2c2ccc(F)cc2)C[C@@H]1N]

### Synthesis of tert-butyl ((2R,4S,5S)-2-((S)-1-(4-fluorophenyl)-1,2,3,4-tetrahydroisoquinoline-2-carbonyl)-5-methoxytetrahydro-2H-pyran-4-yl)carbamate

Tetrabutylammonium hydrogen sulfate (15 mg, 0.043 mmol) followed by aqueous NaOH (50 wt %, 119 μL, 2.23 mmol) and methyl iodide (14.9 μL, 0.238 mmol) were added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 70 mg, 0.149 mmol) in toluene (1.2 mL). After stirring for 3 hours, additional amounts of aqueous NaOH (50 wt %, 119 μL, 2.23 mmol) and methyl iodide (14.9 μL, 0.238 mmol) were added and stirring was continued overnight. The reaction mixture was diluted with water (4 mL) and dichloromethane (4 mL). The layers were separated using a phase-separator and the organic filtrate was evaporated under reduced pressure. The residue was purified by flash column chromatography (silica, 0 to 50% ethyl acetate in heptane) to give tert-butyl ((2R,4S,5S)-2-((S)-1-(4-fluorophenyl)-1,2,3,4-tetrahydroisoquinoline-2-carbonyl)-5-methoxytetrahydro-2H-pyran-4-yl)carbamate.

LCMS: 99%, RT=2.18 min., (M+H)⁺=485 (method A).

### Synthesis of ((2R,4S,5S)-4-amino-5-methoxytetrahydro-2H-pyran-2-yl)((S)-1-(4-fluorophenyl)-3,4-dihydroisoquinolin-2(1H)-yl)methanone (Compound 5062)

Starting from tert-butyl ((2R,4S,5S)-2-((S)-1-(4-fluorophenyl)-1,2,3,4-tetrahydroisoquinoline-2-carbonyl)-5-methoxytetrahydro-2H-pyran-4-yl)carbamate (69 mg, 0.142 mmol), ((2R,4S,5S)-4-amino-5-methoxytetrahydro-2H-pyran-2-yl)((S)-1-(4-fluorophenyl)-3,4-dihydroisoquinolin-2(1H)-yl)methanone (Compound 5062) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.06 min., (M+H)⁺=385 (method P). SFC: RT=3.97 min., (M+H)⁺=385 (method AD).

### Example 46

### Synthesis of ((2R,4S,5S)-4-amino-5-ethoxytetrahydro-2H-pyran-2-yl)((S)-1-(4-fluorophenyl)-3,4-dihydroisoquinolin-2(1H)-yl)methanone (Compound 5063)

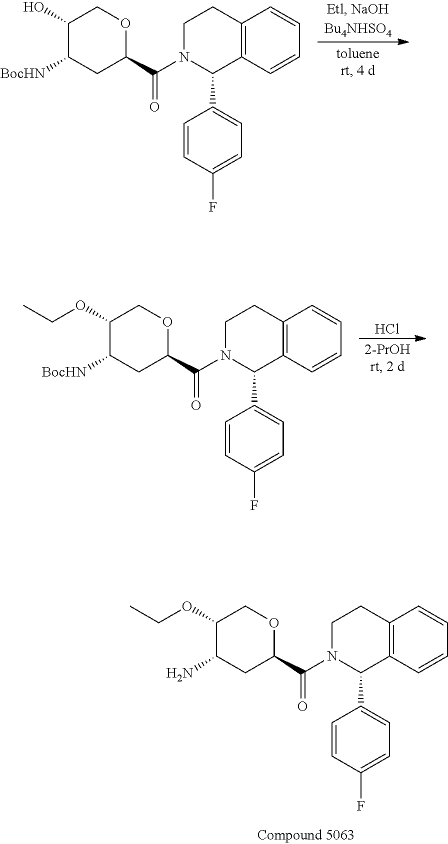 [SMILES: CC(C)(C)OC(=O)N[C@H]1C[C@H](C(=O)N2CCc3ccccc3[C@@H]2c2ccc(F)cc2)OC[C@H]1O ; CCO[C@@H]1CO[C@@H](C(=O)N2CCc3ccccc3[C@@H]2c2ccc(F)cc2)C[C@@H]1NC(=O)OC(C)(C)C ; Cl ; CCO[C@@H]1CO[C@@H](C(=O)N2CCc3ccccc3[C@@H]2c2ccc(F)cc2)C[C@@H]1N]

### Synthesis of tert-butyl ((2R,4S,5S)-5-ethoxy-2-((S)-1-(4-fluorophenyl)-1,2,3,4-tetrahydroisoquinoline-2-carbonyl)tetrahydro-2H-pyran-4-yl)carbamate

Starting from tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 70 mg, 0.149 mmol) and ethyl iodide (19.0 μL, 0.238 mmol), tert-butyl ((2R,4S,5S)-5-ethoxy-2-((S)-1-(4-fluorophenyl)-1,2,3,4-tetrahydroisoquinoline-2-carbonyl)tetrahydro-2H-pyran-4-yl)carbamate was prepared as described for tert-butyl ((2R,4S,5S)-2-((S)-1-(4-fluorophenyl)-1,2,3,4-tetrahydroisoquinoline-2-carbonyl)-5-methoxytetrahydro-2H-pyran-4-yl)carbamate (see Compound 5062).

LCMS: 99%, RT=2.24 min., (M+H)⁺=485 (method A).

### Synthesis of ((2R,4S,5S)-4-amino-5-ethoxytetrahydro-2H-pyran-2-yl)((S)-1-(4-fluorophenyl)-3,4-dihydroisoquinolin-2(1H)-yl)methanone (Compound 5063)

Starting from tert-butyl ((2R,4S,5S)-5-ethoxy-2-((S)-1-(4-fluorophenyl)-1,2,3,4-tetrahydroisoquinoline-2-carbonyl)tetrahydro-2H-pyran-4-yl)carbamate (69 mg, 0.142 mmol), ((2R,4S,5S)-4-amino-5-ethoxytetrahydro-2H-pyran-2-yl)((S)-1-(4-fluorophenyl)-3,4-dihydroisoquinolin-2(1H)-yl)methanone (Compound 5063) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.10 min., (M+H)⁺=399 (method P). SFC: RT=3.91 min., (M+H)⁺=399 (method F).

### Example 47

### Synthesis of ((2R,4S,5R)-5-amino-4-fluorotetrahydro-2H-pyran-2-yl)((S)-1-(4-fluorophenyl)-3,4-dihydroisoquinolin-2(1H)-yl)methanone (Compound 5064)

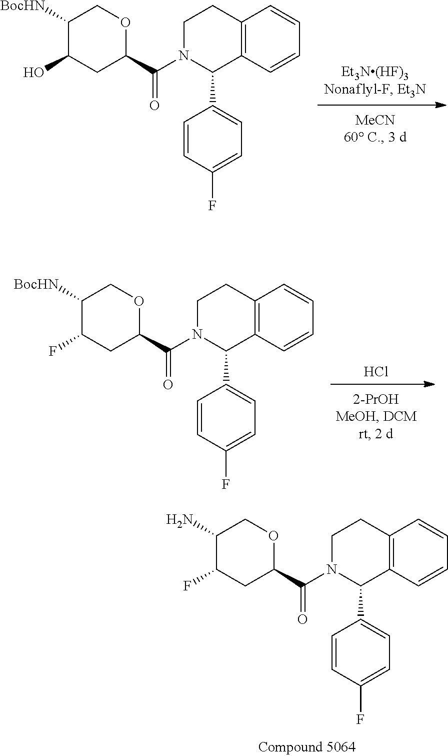

### Synthesis of tert-butyl ((3R,4S,6R)-4-fluoro-6-((S)-1-(4-fluorophenyl)-1,2,3,4-tetrahydroisoquinoline-2-carbonyl)tetrahydro-2H-pyran-3-yl)carbamate

Starting from tert-butyl ((3R,4R,6R)-6-((S)-1-(4-fluorophenyl)-1,2,3,4-tetrahydroisoquinoline-2-carbonyl)-4-hydroxytetrahydro-2H-pyran-3-yl)carbamate (See Compound 5057, 114 mg, 0.242 mmol), tert-butyl ((3R,4S,6R)-4-fluoro-6-((S)-1-(4-fluorophenyl)-1,2,3,4-tetrahydroisoquinoline-2-carbonyl)tetrahydro-2H-pyran-3-yl)carbamate was prepared as described for tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate (see Compound 5069).

LCMS: 95%, RT=2.19 min., (M+H)⁺=473 (method A).

### Synthesis of ((2R,4S,5R)-5-amino-4-fluorotetrahydro-2H-pyran-2-yl)((S)-1-(4-fluorophenyl)-3,4-dihydroisoquinolin-2(1H)-yl)methanone (Compound 5064)

Starting from tert-butyl ((3R,4S,6R)-4-fluoro-6-((S)-1-(4-fluorophenyl)-1,2,3,4-tetrahydroisoquinoline-2-carbonyl)tetrahydro-2H-pyran-3-yl)carbamate (50 mg, 0.106 mmol), methanol and dichloromethane (to solubilize the starting material), ((2R,4S,5R)-5-amino-4-fluorotetrahydro-2H-pyran-2-yl)((S)-1-(4-fluorophenyl)-3,4-dihydroisoquinolin-2(1H)-yl)methanone (Compound 5064) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043) and purified by flash column chromatography (silica, 0 to 2% (7 M ammonia in methanol) in dichloromethane).

LCMS: 98%, RT=1.04 min., (M+H)⁺=373 (method P). SFC: RT=3.43 min., (M+H)⁺=373 (method AD).

### Example 48

### Synthesis of ((2R,4R,5S)-5-amino-4-hydroxytetrahydro-2H-pyran-2-yl)((S)-1-(4-fluorophenyl)-3,4-dihydroisoquinolin-2(1H)-yl)methanone (Compound 5065)

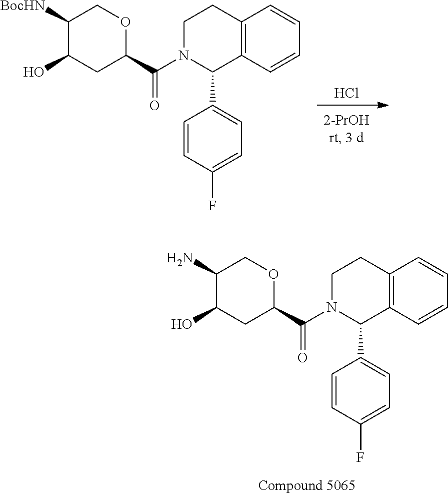 [SMILES: CC(C)(C)OC(=O)N[C@H]1CO[C@@H](C(=O)N2CCc3ccccc3[C@@H]2c2ccc(F)cc2)C[C@H]1O ; Cl ; N[C@H]1CO[C@@H](C(=O)N2CCc3ccccc3[C@@H]2c2ccc(F)cc2)C[C@H]1O]

### Synthesis of ((2R,4R,5S)-5-amino-4-hydroxytetrahydro-2H-pyran-2-yl)((S)-1-(4-fluorophenyl)-3,4-dihydroisoquinolin-2(1H)-yl)methanone (Compound 5065)

Starting from tert-butyl ((3S,4R,6R)-6-((S)-1-(4-fluorophenyl)-1,2,3,4-tetrahydroisoquinoline-2-carbonyl)-4-hydroxytetrahydro-2H-pyran-3-yl)carbamate (See Compound 5052, 50 mg, 0.106 mmol), ((2R,4R,5S)-5-amino-4-hydroxytetrahydro-2H-pyran-2-yl)((S)-1-(4-fluorophenyl)-3,4-dihydroisoquinolin-2(1H)-yl)methanone (Compound 5065) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.00 min., (M+H)⁺=371 (method P). SFC: RT=4.70 min., (M+H)⁺=371 (method F).

### Example 49

### Synthesis of ((S)-1-(4-fluorophenyl)-3,4-dihydroisoquinolin-2(1H)-yl)((2S,4R)-4-((2-hydroxyethyl)amino)tetrahydrofuran-2-yl)methanone (Compound 5066) and ((S)-1-(4-fluorophenyl)-3,4-dihydroisoquinolin-2(1H)-yl)((2S,4S)-4-((2-hydroxyethyl)amino)tetrahydrofuran-2-yl)methanone (Compound 5067)

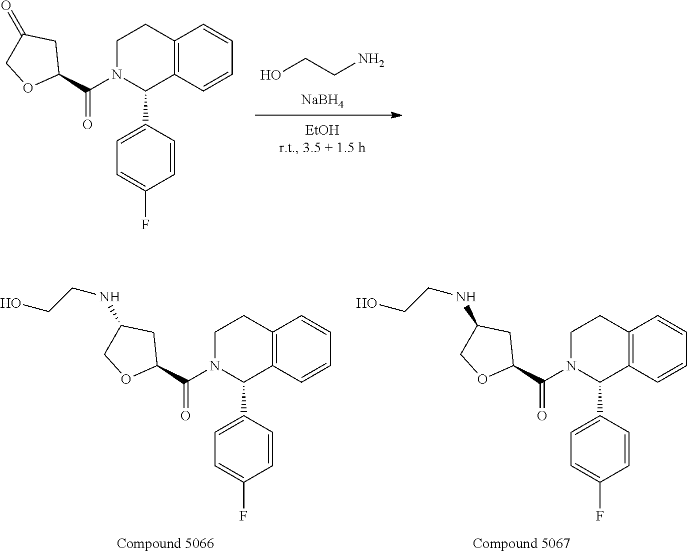

### Synthesis of ((S)-1-(4-fluorophenyl)-3,4-dihydroisoquinolin-2(1H)-yl)((2S,4R)-4-((2-hydroxyethyl)amino)tetrahydrofuran-2-yl)methanone (Compound 5066) and ((S)-1-(4-fluorophenyl)-3,4-dihydroisoquinolin-2(1H)-yl)((2S,4S)-4-((2-hydroxyethyl)amino)tetrahydrofuran-2-yl)methanone (Compound 5067)

Under nitrogen atmosphere, ethanolamine (0.029 mL, 0.486 mmol) was added to a solution of (S)-5-((S)-1-(4-fluorophenyl)-1,2,3,4-tetrahydroisoquinoline-2-carbonyl)dihydrofuran-3(2H)-one (see Compound 5004, 150 mg, 0.442 mmol) in ethanol (absolute, 3.0 mL). After 3.5 hours, NaBH₄ (21.74 mg, 0.575 mmol) was added and stirring was continued for another 1.5 hour. The reaction mixture was diluted with water (1 mL), ethyl acetate (15 mL), and brine (5 mL). The layers were separated, and the aqueous layer was extracted with ethyl acetate (10 mL). The combined organic phase was dried over Na₂SO₄ and evaporated under reduced pressure. The residue was purified by flash column chromatography (silica, 1 to 7% (7M ammonia in methanol) in dichloromethane). The product (109 mg) was combined with the product of a similar reaction (64 mg) and purified by preparative chiral SFC (method BN) to give ((S)-1-(4-fluorophenyl)-3,4-dihydroisoquinolin-2(1H)-yl)((2S,4R)-4-((2-hydroxyethyl)amino)tetrahydrofuran-2-yl)methanone (Compound 5066) as the first eluting isomer after lyophilization from a mixture of water and acetonitrile (1:3, 3 mL) and ((S)-1-(4-fluorophenyl)-3,4-dihydroisoquinolin-2(1H)-yl)((2S,4S)-4-((2-hydroxyethyl)amino)tetrahydrofuran-2-yl)methanone (Compound 5067) as the second eluting isomer after lyophilization from a mixture of water and acetonitrile (1:3, 3 mL).

First eluting isomer: LCMS: 96%, RT=1.00 min., (M+H)⁺=385 (method P). SFC: RT=3.73 min., (M+H)⁺=385 (method AD).

Second eluting isomer: LCMS: 97%, RT=1.01 min., (M+H)⁺=385 (method P). SFC: RT=4.31 min., (M+H)⁺=385 (method AD).

### Example 50

### Synthesis of ((2S,4R,5S)-4-amino-5-hydroxytetrahydro-2H-pyran-2-yl)((S)-1-(4-fluorophenyl)-3,4-dihydroisoquinolin-2(1H)-yl)methanone (Compound 5068)

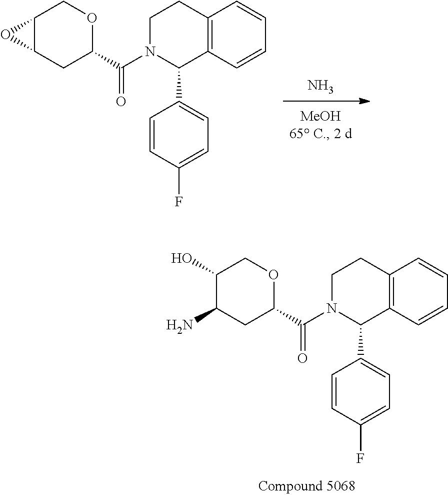

### Synthesis of ((2S,4R,5S)-4-amino-5-hydroxytetrahydro-2H-pyran-2-yl)((S)-1-(4-fluorophenyl)-3,4-dihydroisoquinolin-2(1H)-yl)methanone (Compound 5068)

Starting from ((1R,4R,6S)-3,7-dioxabicyclo[4.1.0]heptan-4-yl)((S)-1-(4-fluorophenyl)-3,4-dihydroisoquinolin-2(1H)-yl)methanone (See Compound 5072, 75 mg, 0.212 mmol), ((2S,4R,5S)-4-amino-5-hydroxytetrahydro-2H-pyran-2-yl)((S)-1-(4-fluorophenyl)-3,4-dihydroisoquinolin-2(1H)-yl)methanone (Compound 5068) was prepared as described for ((2S,4R,5R)-5-amino-4-hydroxytetrahydro-2H-pyran-2-yl)((S)-1-(4-fluorophenyl)-3,4-dihydroisoquinolin-2(1H)-yl)methanone (see Compound 5070) without additional purification by preparative chiral SFC.

LCMS: 98%, RT=1.03 min., (M+H)⁺=371 (method P). SFC: RT=3.65 min., (M+H)⁺=371 (method W).

### Example 51

### Synthesis of ((2R,4S,5R)-4-amino-5-fluorotetrahydro-2H-pyran-2-yl)((S)-1-(4-fluorophenyl)-3,4-dihydroisoquinolin-2(1H)-yl)methanone (Compound 5069)

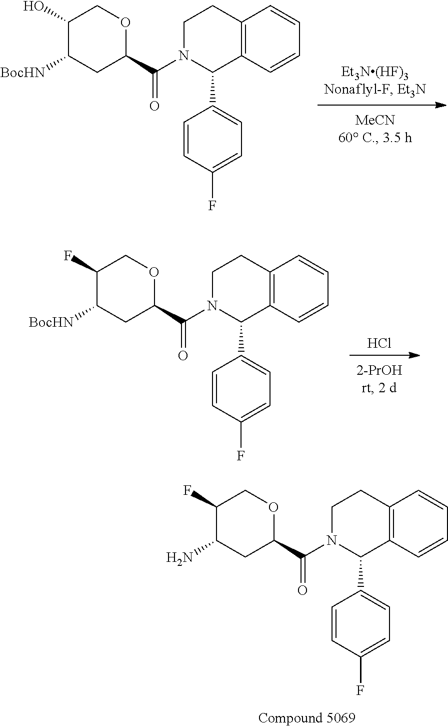

### Synthesis of tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate

Under an argon atmosphere in a microwave vial, triethylamine trihydrofluoride (0.076 mL, 0.468 mmol) and perfluoro-1-butanesulfonyl fluoride (0.084 mL, 0.468 mmol) were added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (See Compound 5052, 100 mg, 0.213 mmol) and triethylamine (0.196 mL, 1.407 mmol) in dry acetonitrile (1.5 mL). The vial was sealed, transferred into pre-heated oil bath of 60° C., and stirred for 3.5 hours. After cooling the mixture to room temperature, it was poured onto a mixture of saturated aqueous NaHCO₃ and ice (10 mL). The aqueous layer was extracted with ethyl acetate (2×10 mL) and the combined phase was dried over Na₂SO₄ and evaporated under reduced pressure. The residue was purified by flash column chromatography (silica, 0 to 35% ethyl acetate in heptane) to give tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate.

LCMS: 99%, RT=2.18 min., (M+H)⁺=473 (method A).

### Synthesis of ((2R,4S,5R)-4-amino-5-fluorotetrahydro-2H-pyran-2-yl)((S)-1-(4-fluorophenyl)-3,4-dihydroisoquinolin-2(1H)-yl)methanone (Compound 5069)

Starting from tert-butyl ((2R,4S,5R)-5-fluoro-2-((S)-1-(4-fluorophenyl)-1,2,3,4-tetrahydroisoquinoline-2-carbonyl)tetrahydro-2H-pyran-4-yl)carbamate (36 mg, 0.076 mmol), ((2R,4S,5R)-4-amino-5-fluorotetrahydro-2H-pyran-2-yl)((S)-1-(4-fluorophenyl)-3,4-dihydroisoquinolin-2(1H)-yl)methanone (Compound 5069) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.07 min., (M+H)⁺=373 (method P). SFC: RT=3.15 min., (M+H)⁺=373 (method AD).

### Example 52

### Synthesis of ((2R,4S,5S)-5-amino-4-hydroxytetrahydro-2H-pyran-2-yl)((S)-1-(4-fluorophenyl)-3,4-dihydroisoquinolin-2(1H)-yl)methanone (Compound 5070)

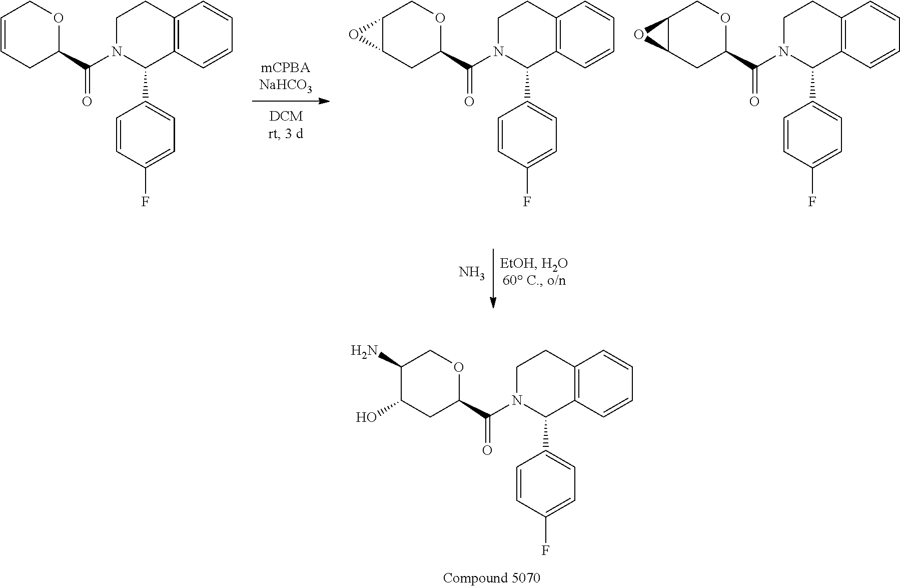

### Synthesis of ((1R,4R,6S)-3,7-dioxabicyclo[4.1.0]heptan-4-yl)((S)-1-(4-fluorophenyl)-3,4-dihydroisoquinolin-2(1H)-yl)methanone and ((1S,4R,6R)-3,7-dioxabicyclo[4.1.0]heptan-4-yl)((S)-1-(4-fluorophenyl)-3,4-dihydroisoquinolin-2(1H)-yl)methanone

m-Chloroperbenzoic acid (70%, 4.09 g, 16.60 mmol) was added portion wise to a mixture of ((R)-3,6-dihydro-2H-pyran-2-yl)((S)-1-(4-fluorophenyl)-3,4-dihydroisoquinolin-2(1H)-yl)methanone (See Compound 5053, 2.8 g, 8.30 mmol) and NaHCO₃ (2.091 g, 24.90 mmol) in dichloromethane (50 mL). After stirring the mixture for 3 days, it was diluted with dichloromethane (300 mL) and washed with saturated aqueous NaHCO₃. The aqueous phase was extracted with dichloromethane and the combined organic layers were washed with saturated aqueous NaHCO₃ and brine, dried over Na₂SO₄ and evaporated under reduced pressure. The residue was purified by flash column chromatography (silica, 0 to 100% ethyl acetate in heptane) to give ((1R,4R,6S)-3,7-dioxabicyclo[4.1.0]heptan-4-yl)((S)-1-(4-fluorophenyl)-3,4-dihydroisoquinolin-2(1H)-yl)methanone as the first eluting isomer and ((1S,4R,6R)-3,7-dioxabicyclo[4.1.0]heptan-4-yl)((S)-1-(4-fluorophenyl)-3,4-dihydroisoquinolin-2(1H)-yl)methanone as the second eluting isomer.

First eluting isomer: LCMS: 88%, RT=2.01 min., (M+H)⁺=354 (method A).

Second eluting isomer: LCMS: 95%, RT=1.96 min., (M+H)⁺=354 (method A).

### Synthesis of ((2R,4S,5S)-5-amino-4-hydroxytetrahydro-2H-pyran-2-yl)((S)-1-(4-fluorophenyl)-3,4-dihydroisoquinolin-2(1H)-yl)methanone (Compound 5070)

Ammonia (32% in water, 517 μL, 8.55 mmol) was added to a solution of ((1R,4R,6S)-3,7-dioxabicyclo[4.1.0]heptan-4-yl)((S)-1-(4-fluorophenyl)-3,4-dihydroisoquinolin-2(1H)-yl)methanone (302 mg, 0.855 mmol) in ethanol (12 mL) and the mixture was stirred at 60° C. overnight. The reaction mixture was concentrated to dryness under reduced pressure. The residue was purified by acidic preparative MPLC (Linear Gradient: t=0 min 5% A; t=1 min 10% A; t=16 min 50% A; t=17 min 100%; t=22 min 100% A; detection: 220 nm) and preparative chiral SFC (method AN) to give ((2R,4S,5S)-5-amino-4-hydroxytetrahydro-2H-pyran-2-yl)((S)-1-(4-fluorophenyl)-3,4-dihydroisoquinolin-2(1H)-yl)methanone (Compound 5070) after lyophilization from a mixture of acetonitrile and water (1:1).

LCMS: 99%, RT=1.00 min., (M+H)⁺=371 (method P). SFC: RT=3.23 min., (M+H)⁺=371 (method AD).

### Example 53

### Synthesis of ((2R,4S)-4-amino-4-(hydroxymethyl)tetrahydrofuran-2-yl)((S)-1-(4-fluorophenyl)-3,4-dihydroisoquinolin-2(1H)-yl)methanone (Compound 5071)

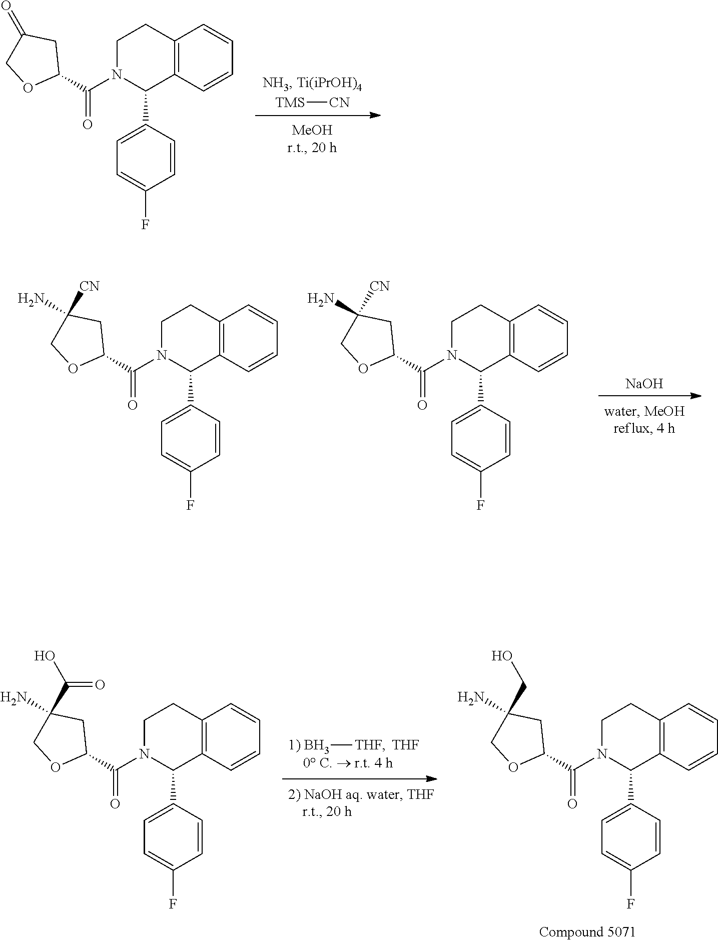

### Synthesis of (3S,5R)-3-amino-5-((S)-1-(4-fluorophenyl)-1,2,3,4-tetrahydroisoquinoline-2-carbonyl)tetrahydrofuran-3-carbonitrile

Titanium(IV) isopropoxide (0.109 mL, 0.368 mmol) was added to a solution of (R)-5-((S)-1-(4-fluorophenyl)-1,2,3,4-tetrahydroisoquinoline-2-carbonyl)dihydrofuran-3(2H)-one (see Compound 50004, 104 mg, 0.306 mmol) in a mixture of methanol (extra dry, 2 mL) and ammonia in methanol (7 M, 2 mL). After stirring for 4 hours, trimethylsilyl cyanide (0.041 mL, 0.306 mmol) was added and stirring was continued for 16 hours. The reaction mixture was diluted with saturated aqueous NaHCO₃ (0.5 mL) and extracted with dichloromethane (20 mL). The layers were separated using a phase-separator and the organic filtrate evaporated under reduced pressure. The residue was purified by flash column chromatography (silica, 0 to 100% ethyl acetate in heptane) to give (3S,5R)-3-amino-5-((S)-1-(4-fluorophenyl)-1,2,3,4-tetrahydroisoquinoline-2-carbonyl)tetrahydrofuran-3-carbonitrile as the first eluting isomer and (3R,5R)-3-amino-5-((S)-1-(4-fluorophenyl)-1,2,3,4-tetrahydroisoquinoline-2-carbonyl)tetrahydrofuran-3-carbonitrile as the second eluting isomer. The configuration of the tetrahydrofuran-amine stereocenter was arbitrarily assigned

First eluting isomer: LCMS: 98%, RT=1.87 min., (M+H)⁺=366 (method A), SFC: 99%, RT=3.49 min., (M+H)⁺=366 (method W).

Second eluting isomer: LCMS: 87%, RT=1.86 min., (M+H)⁺=366 (method A). SFC: 99%, RT=4.06 min., (M+H)⁺=366 (method W).

### Synthesis of (3R,5R)-3-amino-5-((S)-1-(4-fluorophenyl)-1,2,3,4-tetrahydroisoquinoline-2-carbonyl)tetrahydrofuran-3-carboxylic acid

Sodium hydroxide (85 mg, 2.14 mmol) was added to a solution of (3S,5R)-3-amino-5-((S)-1-(4-fluorophenyl)-1,2,3,4-tetrahydroisoquinoline-2-carbonyl)tetrahydrofuran-3-carbonitrile (78 mg, 0.213 mmol) in a mixture of methanol and water (1:1, 2 mL) and the mixture was stirred at 65° C. for 4 hours. The volatiles were removed under reduced pressure and aqueous HCl (1 M) was added until pH-5. The mixture was extracted with ethyl acetate (4 mL), dried over Na₂SO₄, and evaporated under reduced pressure to give (3R,5R)-3-amino-5-((S)-1-(4-fluorophenyl)-1,2,3,4-tetrahydroisoquinoline-2-carbonyl)tetrahydrofuran-3-carboxylic acid. The configuration of the tetrahydrofuran-amine stereocenter was arbitrarily assigned

LCMS: 89%, RT=1.72 min., (M+H)⁺=385 (method A).

### Synthesis of ((2R,4S)-4-amino-4-(hydroxymethyl)tetrahydrofuran-2-yl)((S)-1-(4-fluorophenyl)-3,4-dihydroisoquinolin-2(1H)-yl)methanone (Compound 5071)

At 0° C., borane-tetrahydrofuran complex (1 M in tetrahydrofuran, 0.293 mL, 0.293 mmol) was added to a solution of (3R,5R)-3-amino-5-((S)-1-(4-fluorophenyl)-1,2,3,4-tetrahydroisoquinoline-2-carbonyl)tetrahydrofuran-3-carboxylic acid (45 mg, 0.117 mmol) in tetrahydrofuran (dry, 4 mL). After 4 hours, aqueous NaOH (3 M, 1 mL) was added and stirring was continued for 16 hours. The reaction mixture was saturated with Na₂CO₃ and the product was extracted with ethyl acetate (10 mL). The organic layer was dried over Na₂SO₄ and evaporated under reduced pressure. The residue was purified by acidic preparative MPLC (Linear Gradient: t=0 min 5% A; t=1 min 5% A; t=16 min 40% A; t=17 min 100%; t=22 min 100% A; detection: 220/216 nm). The residue was dissolved in methanol (2 mL) and brought onto an SCX-2 column (1 g) and eluted with methanol until neutral. Next, the column was eluted with ammonia in methanol (3 M). The basic fraction was concentrated to dryness under reduced pressure and the residue lyophilized from a mixture of acetonitrile and water (1:1, 4 mL) to give ((2R,4S)-4-amino-4-(hydroxymethyl)tetrahydrofuran-2-yl)((S)-1-(4-fluorophenyl)-3,4-dihydroisoquinolin-2(1H)-yl)methanone (Compound 5071). The configuration of the tetrahydrofuran-amine stereocenter was arbitrarily assigned

LCMS: 95%, RT=1.01 min., (M+H)⁺=371 (method P). SFC: 97%, RT=3.45 min., (M+H)⁺=371 (method W).

### Example 54

### Synthesis of ((2S,4R,5R)-5-amino-4-hydroxytetrahydro-2H-pyran-2-yl)((S)-1-(4-fluorophenyl)-3,4-dihydroisoquinolin-2(1H)-yl)methanone (Compound 5072)

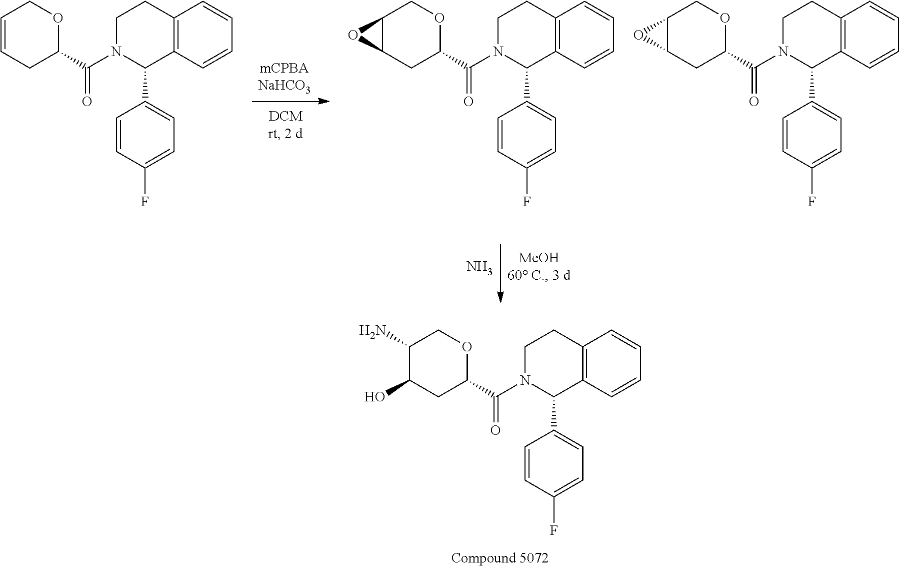

### Synthesis of ((1S,4S,6R)-3,7-dioxabicyclo[4.1.0]heptan-4-yl)((S)-1-(4-fluorophenyl)-3,4-dihydroisoquinolin-2(1H)-yl)methanone and ((1R,4S,6S)-3,7-dioxabicyclo[4.1.0]heptan-4-yl)((S)-1-(4-fluorophenyl)-3,4-dihydroisoquinolin-2(1H)-yl)methanone

Starting from ((S)-3,6-dihydro-2H-pyran-2-yl)((S)-1-(4-fluorophenyl)-3,4-dihydroisoquinolin-2(1H)-yl)methanone (2.71 g, 8.03 mmol), ((1S,4S,6R)-3,7-dioxabicyclo[4.1.0]heptan-4-yl)((S)-1-(4-fluorophenyl)-3,4-dihydroisoquinolin-2(1H)-yl)methanone as the first eluting isomer on silica and ((1R,4S,6S)-3,7-dioxabicyclo[4.1.0]heptan-4-yl)((S)-1-(4-fluorophenyl)-3,4-dihydroisoquinolin-2(1H)-yl)methanone as the second eluting isomer on silica were prepared as described for ((1R,4R,6S)-3,7-dioxabicyclo[4.1.0]heptan-4-yl)((S)-1-(4-fluorophenyl)-3,4-dihydroisoquinolin-2(1H)-yl)methanone and ((1S,4R,6R)-3,7-dioxabicyclo[4.1.0]heptan-4-yl)((S)-1-(4-fluorophenyl)-3,4-dihydroisoquinolin-2(1H)-yl)methanone.

First eluting isomer: LCMS: 84%, RT=2.00 min., (M+H)⁺=354 (method A).

Second eluting isomer: LCMS: 96%, RT=1.97 min., (M+H)⁺=354 (method A).

### Synthesis of ((2S,4R,5R)-5-amino-4-hydroxytetrahydro-2H-pyran-2-yl)((S)-1-(4-fluorophenyl)-3,4-dihydroisoquinolin-2(1H)-yl)methanone (Compound 5072)

((1S,4S,6R)-3,7-dioxabicyclo[4.1.0]heptan-4-yl)((S)-1-(4-fluorophenyl)-3,4-dihydroisoquinolin-2(1H)-yl)methanone (200 mg, 0.447 mmol) was dissolved in ammonia (7 M in methanol, 10 mL, 70 mmol). The reaction vial was sealed and warmed to 60° C. and stirred for 3 days. The reaction mixture was concentrated to dryness under reduced pressure. The residue was purified by flash column chromatography (silica, 0.5 to 8% (7M ammonia in methanol) in dichloromethane) and part of the product (45 mg out of 145 mg) was dissolved in methanol, brought onto an SCX-2 column (6 g) and eluted with methanol until neutral. Next, the column was eluted with ammonia in methanol (1 M). The basic fraction was concentrated to dryness under reduced pressure. The product (35 mg) was combined with a similarly prepared batch (53 mg) and purified by preparative chiral SFC (method AN) to give ((2S,4R,5R)-5-amino-4-hydroxytetrahydro-2H-pyran-2-yl)((S)-1-(4-fluorophenyl)-3,4-dihydroisoquinolin-2(1H)-yl)methanone (Compound 5072) after lyophilization from a mixture of acetonitrile and water (1:3, 3 mL).

LCMS: 98%, RT=1.01 min., (M+H)⁺=371 (method P). SFC: RT=3.35 min., (M+H)⁺=371 (method AD).

### Example 55

### Synthesis of ((2R,4R,5R)-4-amino-5-methoxytetrahydro-2H-pyran-2-yl)((S)-1-(4-fluorophenyl)-3,4-dihydroisoquinolin-2(1H)-yl)methanone (Compound 5073)

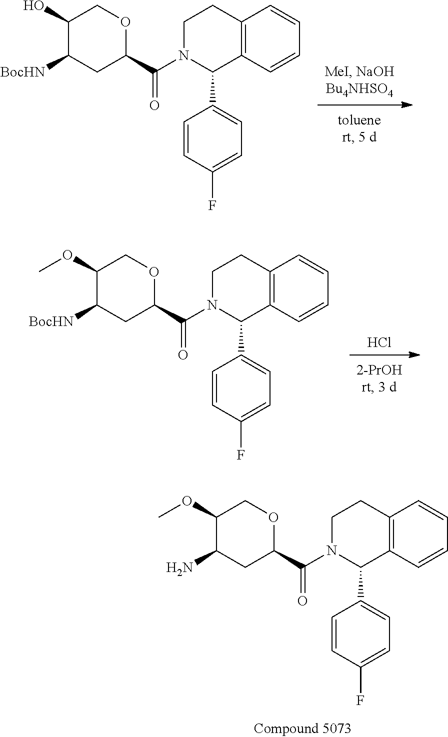

### Synthesis of tert-butyl ((2R,4R,5R)-2-((S)-1-(4-fluorophenyl)-1,2,3,4-tetrahydroisoquinoline-2-carbonyl)-5-methoxytetrahydro-2H-pyran-4-yl)carbamate

Starting from tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 35 mg, 0.074 mmol), tert-butyl ((2R,4R,5R)-2-((S)-1-(4-fluorophenyl)-1,2,3,4-tetrahydroisoquinoline-2-carbonyl)-5-methoxytetrahydro-2H-pyran-4-yl)carbamate was prepared as described for tert-butyl ((2R,4S,5S)-2-((S)-1-(4-fluorophenyl)-1,2,3,4-tetrahydroisoquinoline-2-carbonyl)-5-methoxytetrahydro-2H-pyran-4-yl)carbamate (see Compound 5062).

LCMS: 95%, RT=2.15 min., (M+Na)⁺=507 (method A).

### Synthesis of ((2R,4R,5R)-4-amino-5-methoxytetrahydro-2H-pyran-2-yl)((S)-1-(4-fluorophenyl)-3,4-dihydroisoquinolin-2(1H)-yl)methanone (Compound 5073)

Starting from tert-butyl ((2R,4R,5R)-2-((S)-1-(4-fluorophenyl)-1,2,3,4-tetrahydroisoquinoline-2-carbonyl)-5-methoxytetrahydro-2H-pyran-4-yl)carbamate (34 mg, 0.070 mmol), ((2R,4R,5R)-4-amino-5-methoxytetrahydro-2H-pyran-2-yl)((S)-1-(4-fluorophenyl)-3,4-dihydroisoquinolin-2(1H)-yl)methanone (Compound 5073) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.05 min., (M+H)⁺=385 (method P).

### Example 56

### Synthesis of ((2R,4R,5R)-4-amino-5-hydroxytetrahydro-2H-pyran-2-yl)((S)-1-(4-fluorophenyl)-3,4-dihydroisoquinolin-2(1H)-yl)methanone (Compound 5074)

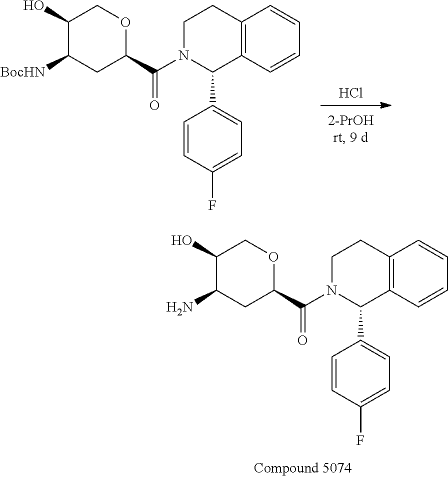

### Synthesis of ((2R,4R,5R)-4-amino-5-hydroxytetrahydro-2H-pyran-2-yl)((S)-1-(4-fluorophenyl)-3,4-dihydroisoquinolin-2(1H)-yl)methanone (Compound 5074)

Starting from tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 22 mg, 0.047 mmol), ((2R,4R,5R)-4-amino-5-hydroxytetrahydro-2H-pyran-2-yl)((S)-1-(4-fluorophenyl)-3,4-dihydroisoquinolin-2(1H)-yl)methanone (Compound 5074) was prepared as described for ((2S,4R,5S)-5-amino-4-hydroxytetrahydro-2H-pyran-2-yl)((S)-1-(4-fluorophenyl)-3,4-dihydroisoquinolin-2(1H)-yl)methanone (Compound 5043).

LCMS: 99%, RT=1.00 min., (M+H)⁺=371 (method P).

### Example 57

### Synthesis of ((2R,4R)-4-aminotetrahydro-2H-pyran-2-yl)((S)-1-(4-fluorophenyl)-3,4-dihydroisoquinolin-2(1H)-yl)methanone (Compound 5028) and ((2R,4S)-4-aminotetrahydro-2H-pyran-2-yl)((S)-1-(4-fluorophenyl)-3,4-dihydroisoquinolin-2(1H)_-yl)methanone (Compound 5080)

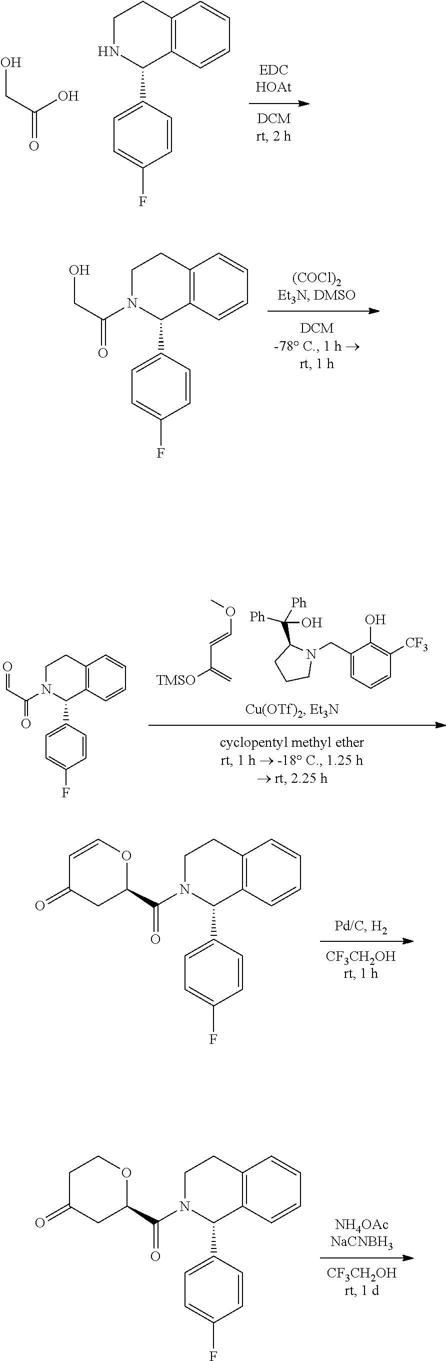

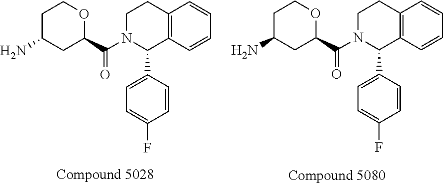 [SMILES: N[C@@H]1CCO[C@@H](C(=O)N2CCc3ccccc3[C@@H]2c2ccc(F)cc2)C1 ; N[C@H]1CCO[C@@H](C(=O)N2CCc3ccccc3[C@@H]2c2ccc(F)cc2)C1]

### Synthesis of (S)-1-(1-(4-fluorophenyl)-3,4-dihydroisoquinolin-2(1H)-yl)-2-hydroxyethan-1-one

N-(3-dimethylaminopropyl)-N′-ethylcarbodiimide hydrochloride (2.320 g, 12.10 mmol) and 1-hydroxy-7-azabenzotriazole (0.150 g, 1.100 mmol) were added to a solution of (S)-1-(4-fluorophenyl)-1,2,3,4-tetrahydroisoquinoline (2.5 g, 11.00 mmol) and glycolic acid (0.725 mL, 12.10 mmol) in dichloromethane (20 mL). After 2 hours, the mixture was diluted with water and the layers were separated over a phase separation filter. The organic layer was evaporated under reduced pressure. The residue was purified by flash column chromatography (silica, 10 to 60% ethyl acetate in heptane) to give (S)-1-(1-(4-fluorophenyl)-3,4-dihydroisoquinolin-2(1H)-yl)-2-hydroxyethan-1-one.

LCMS: 99%, RT=1.90 min., (M+H)⁺=286 (method A).

### Synthesis of (S)-2-(1-(4-fluorophenyl)-3,4-dihydroisoquinolin-2(1H)-yl)-2-oxoacetaldehyde

Under nitrogen atmosphere at −78° C., a solution of dimethyl sulfoxide (0.850 mL, 11.97 mmol) in dichloromethane (15.0 mL) was added dropwise to a solution of oxalyl chloride (0.685 mL, 7.98 mmol) in dichloromethane (dry, 7.5 mL). After 20 minutes, a solution of (S)-1-(1-(4-fluorophenyl)-3,4-dihydroisoquinolin-2(1H)-yl)-2-hydroxyethan-1-one (1.138 g, 3.99 mmol) in dichloromethane (7.5 mL) was added dropwise and after another 30 minutes, triethylamine (2.77 mL, 19.94 mmol). After 30 minutes, the reaction mixture was allowed to warm to room temperature and stirred for 1 hour. The reaction mixture was partitioned between water (50 mL) and dichloromethane (50 mL) and the aqueous phase was extracted with dichloromethane (15 mL). The combined organic phase was washed with brine (30 mL), dried over Na₂SO₄ and evaporated under reduced pressure. Part of the residue was purified by basic preparative MPLC (Linear Gradient: t=0 min 5% A; t=1 min 5% A; t=2 min 20% A; t=17 min 50% A; t=18 min 100% A; t=23 min 100% A; detection: 220 nm) to give (S)-2-(1-(4-fluorophenyl)-3,4-dihydroisoquinolin-2(1H)-yl)-2-oxoacetaldehyde after lyophilization of the product containing fractions.

LCMS: 97%, RT=1.93 min., (M+H₂O (hydrated ketone)+H)⁺=302 (method B).

### Synthesis of (R)-2-((S)-1-(4-fluorophenyl)-1,2,3,4-tetrahydroisoquinoline-2-carbonyl)-2,3-dihydro-4H-pyran-4-one

Under nitrogen atmosphere in a microwave vial, copper(II) triflate (38.3 mg, 0.106 mmol) was added to a stirred solution of (S)-2-((2-(hydroxydiphenylmethyl)pyrrolidin-1-yl)methyl)-6-(trifluoromethyl)phenol (45.3 mg, 0.106 mmol) and triethylamine (0.015 mL, 0.106 mmol) in cyclopentyl methyl ether (8.0 mL). The reaction vial was sealed with a septum and stirred for 1 hour. (S)-2-(1-(4-fluorophenyl)-3,4-dihydroisoquinolin-2(1H)-yl)-2-oxoacetaldehyde (200 mg, 0.706 mmol) and powdered 4 Å molecular sieves (400 mg) were added and the reaction mixture was cooled to −18° C. and stirred for 30 minutes. Then (E)-((4-methoxybuta-1,3-dien-2-yl)oxy)trimethylsilane (0.241 mL, 1.235 mmol) was added dropwise. Stirring was continued for 45 minutes, after the mixture was allowed to slowly warm to room temperature. After 2.25 hours, the reaction mixture was cooled in an ice/water bath and quenched by the dropwise addition of trifluoroacetic acid (0.135 mL, 1.765 mmol). After 5 minutes, the reaction vial was stored in the freezer overnight. After warming to room temperature, the mixture was diluted with ethyl acetate (10 mL) and filtered through a small pad of Celite. The residue was rinsed with ethyl acetate (2×5 mL) and the combined filtrates were washed with saturated aqueous Na₂CO₃ (12.5 mL). The aqueous phase was extracted with ethyl acetate (12.5 mL). The combined organics were washed with brine (12.5 mL), dried over Na₂SO₄, and evaporated under reduced pressure. The residue was purified by flash column chromatography (silica, 0 to 50% ethyl acetate in heptane) to give (R)-2-((S)-1-(4-fluorophenyl)-1,2,3,4-tetrahydroisoquinoline-2-carbonyl)-2,3-dihydro-4H-pyran-4-one containing a minor amount of (S)-2-((S)-1-(4-fluorophenyl)-1,2,3,4-tetrahydroisoquinoline-2-carbonyl)-2,3-dihydro-4H-pyran-4-one.

LCMS: 95%, RT=1.99 min., (M+H)⁺=352 (method A).

### Synthesis of (R)-2-((S)-1-(4-fluorophenyl)-1,2,3,4-tetrahydroisoquinoline-2-carbonyl)tetrahydro-4H-pyran-4-one

Palladium on carbon (10 wt %, containing 50% water, 72.7 mg, 0.068 mmol) was added to a solution of (R)-2-((S)-1-(4-fluorophenyl)-1,2,3,4-tetrahydroisoquinoline-2-carbonyl)-2,3-dihydro-4H-pyran-4-one (240 mg, 0.683 mmol) in 2,2,2-trifluoroethanol (7.5 mL). The mixture was hydrogenated at room temperature and ambient pressure for 1 hour. The reaction mixture was filtered through a nylon, 0.45 μm filter and the filtrate was evaporated under reduced pressure. The residue was purified by flash column chromatography (silica, 0 to 50% ethyl acetate in heptane) and preparative chiral SFC (method AM) to give (R)-2-((S)-1-(4-fluorophenyl)-1,2,3,4-tetrahydroisoquinoline-2-carbonyl)tetrahydro-4H-pyran-4-one

LCMS: 99%, RT=2.01 min., (M+H)⁺=354 (method A). SFC: RT=3.79 min., (M+H)⁺=354 (method AD

### Synthesis of ((2R,4R)-4-aminotetrahydro-2H-pyran-2-yl)((S)-1-(4-fluorophenyl)-3,4-dihydroisoquinolin-2(1H)-yl)methanone (Compound 5028) and ((2R,4S)-4-aminotetrahydro-2H-pyran-2-yl)((S)-1-(4-fluorophenyl)-3,4-dihydroisoquinolin-2(1H)-yl)methanone (Compound 5080)

Ammonium acetate (262 mg, 3.40 mmol) was added to a solution of (R)-2-((S)-1-(4-fluorophenyl)-1,2,3,4-tetrahydroisoquinoline-2-carbonyl)tetrahydro-4H-pyran-4-one (120 mg, 0.340 mmol) in 2,2,2-trifluoroethanol (4.0 mL). After overnight stirring, sodium cyanoborohydride (64.0 mg, 1.019 mmol) was added and after 2 hours an extra quantity of sodium cyanoborohydride (64.0 mg, 1.019 mmol) was added. After an additional 2 hours, ammonium acetate (262 mg, 3.40 mmol) was added and after another hour, sodium cyanoborohydride (64.0 mg, 1.019 mmol). Stirring was continued for 1 hour after which the reaction mixture was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm) and preparative chiral SFC (method AL) to give ((2R,4R)-4-aminotetrahydro-2H-pyran-2-yl)((S)-1-(4-fluorophenyl)-3,4-dihydroisoquinolin-2(1H)-yl)methanone (Compound 5028) as the first eluting SFC isomer and ((2R,4S)-4-aminotetrahydro-2H-pyran-2-yl)((S)-1-(4-fluorophenyl)-3,4-dihydroisoquinolin-2(1H)-yl)methanone (Compound 5080) as the second eluting SFC isomer, both after lyophilization from a mixture of acetonitrile and water (3:2, 2.5 mL).

Compound 5028: LCMS: 99%, RT=1.06 min., (M+H)⁺=355 (method P). SFC: RT=4.24 min., (M+H)⁺=355 (method F).

Compound 5080: LCMS: 99%, RT=1.04 min., (M+H)⁺=355 (method P). SFC: RT=4.58 min., (M+H)⁺=355 (method F).

### Example 58

### Synthesis of ((2S,4S)-4-aminotetrahydro-2H-pyran-2-yl)((S)-1-(4-fluorophenyl)-3,4-dihydroisoquinolin-2(1H)-yl)methanone (Compound 5027) and ((2S,4R)-4-aminotetrahydro-2H-pyran-2-yl)((S)-1-(4-fluorophenyl)-3,4-dihydroisoquinolin-2(1H)-yl)methanone (Compound 5081)

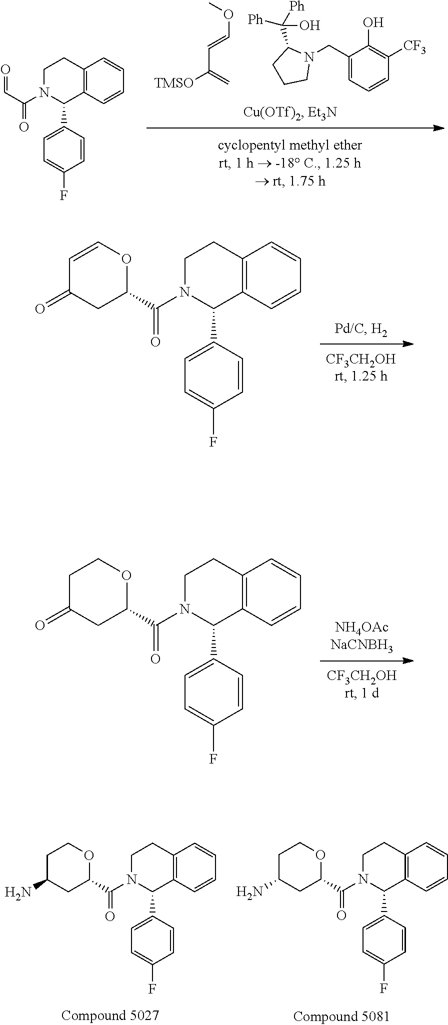

### Synthesis of (S)-2-((S)-1-(4-fluorophenyl)-1,2,3,4-tetrahydroisoquinoline-2-carbonyl)-2,3-dihydro-4H-pyran-4-one

Starting from (R)-2-((2-(hydroxydiphenylmethyl)pyrrolidin-1-yl)methyl)-6-(trifluoromethyl)phenol (74.4 mg, 0.175 mmol) and (S)-2-(1-(4-fluorophenyl)-3,4-dihydroisoquinolin-2(1H)-yl)-2-oxoacetaldehyde (330 mg, 1.165 mmol), (S)-2-((S)-1-(4-fluorophenyl)-1,2,3,4-tetrahydroisoquinoline-2-carbonyl)-2,3-dihydro-4H-pyran-4-one (207 mg) as a mixture with (R)-2-((S)-1-(4-fluorophenyl)-1,2,3,4-tetrahydroisoquinoline-2-carbonyl)-2,3-dihydro-4H-pyran-4-one was prepared as described for (R)-2-((S)-1-(4-fluorophenyl)-1,2,3,4-tetrahydroisoquinoline-2-carbonyl)-2,3-dihydro-4H-pyran-4-one (see Compound 5028).

LCMS: 98%, RT=1.98 min., (M+H)⁺=352 (method A).

### Synthesis of (S)-2-((S)-1-(4-fluorophenyl)-1,2,3,4-tetrahydroisoquinoline-2-carbonyl)tetrahydro-4H-pyran-4-one

Starting from (S)-2-((S)-1-(4-fluorophenyl)-1,2,3,4-tetrahydroisoquinoline-2-carbonyl)-2,3-dihydro-4H-pyran-4-one as a mixture with (R)-2-((S)-1-(4-fluorophenyl)-1,2,3,4-tetrahydroisoquinoline-2-carbonyl)-2,3-dihydro-4H-pyran-4-one (205 mg, 0.583 mmol), (S)-2-((S)-1-(4-fluorophenyl)-1,2,3,4-tetrahydroisoquinoline-2-carbonyl)tetrahydro-4H-pyran-4-one was prepared as described for (R)-2-((S)-1-(4-fluorophenyl)-1,2,3,4-tetrahydroisoquinoline-2-carbonyl)tetrahydro-4H-pyran-4-one (see Compound 5028).

LCMS: 99%, RT=2.00 min., (M+H)⁺=354 (method A). SFC: RT=3.48 min., (M+H)⁺=354 (method AD).

### Synthesis of ((2S,4S)-4-aminotetrahydro-2H-pyran-2-yl)((S)-1-(4-fluorophenyl)-3,4-dihydroisoquinolin-2(1H)-yl)methanone (Compound 5027) and ((2S,4R)-4-aminotetrahydro-2H-pyran-2-yl)((S)-1-(4-fluorophenyl)-3,4-dihydroisoquinolin-2(1H)-yl)methanone (Compound 5081)

Starting from (S)-2-((S)-1-(4-fluorophenyl)-1,2,3,4-tetrahydroisoquinoline-2-carbonyl)tetrahydro-4H-pyran-4-one (80 mg, 0.226 mmol), ((2S,4S)-4-aminotetrahydro-2H-pyran-2-yl)((S)-1-(4-fluorophenyl)-3,4-dihydroisoquinolin-2(1H)-yl)methanone (Compound 5027,) as the first eluting SFC isomer and ((2S,4R)-4-aminotetrahydro-2H-pyran-2-yl)((S)-1-(4-fluorophenyl)-3,4-dihydroisoquinolin-2(1H)-yl)methanone (Compound 5081,) as the second eluting SFC isomer were prepared as described for ((2R,4R)-4-aminotetrahydro-2H-pyran-2-yl)((S)-1-(4-fluorophenyl)-3,4-dihydroisoquinolin-2(1H)-yl)methanone (Compound 5028).

Compound 5027: LCMS: 99%, RT=1.06 min., (M+H)⁺=355 (method P). SFC: RT=3.84 min., (M+H)⁺=355 (method AD).

Compound 5081: LCMS: 99%, RT=1.04 min., (M+H)⁺=355 (method P). SFC: RT=4.11 min., (M+H)⁺=355 (method F).

### Example 59

### Synthesis of Compound 5090 and Compound 5091

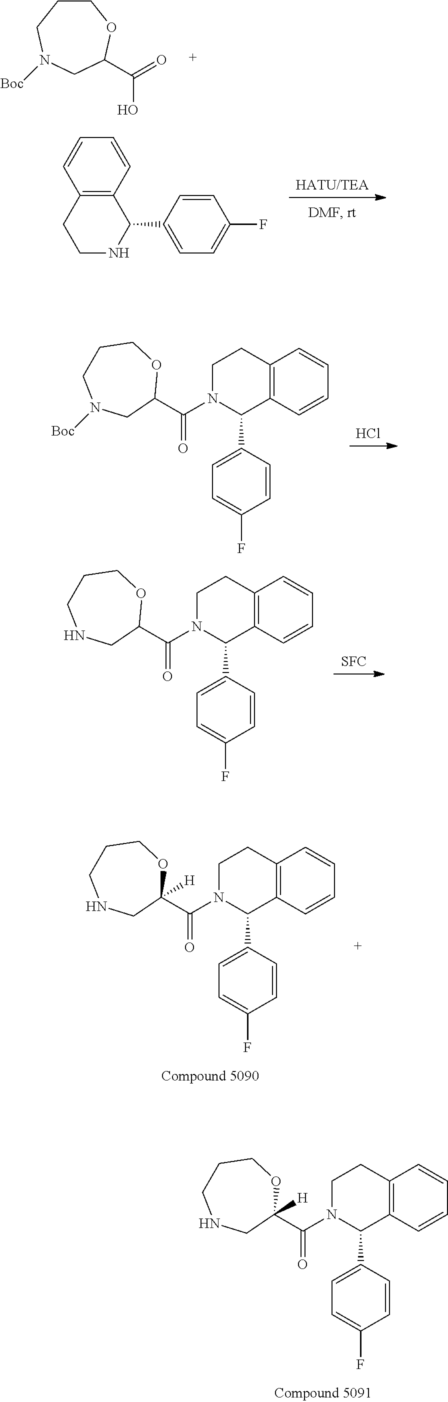

Step 1: To a solution of (S)-1-(4-fluorophenyl)-1,2,3,4-tetrahydroisoquinoline (470 mg, 1.6 mmol) in DMF (5 mL) were added 4-(tert-butoxycarbonyl)-1,4-oxazepane-2-carboxylic acid (200 mg, 0.81 mmol), HATU (456 mg, 1.2 mmol) and TEA (0.3 mL, 2 mmol) at 0° C. The resulting reaction mixture was stirred at room temperature for 2 h, diluted with EA (10 mL) and washed with saturated NH₄Cl (2×10 mL) followed by brine (2×10 mL). The organic phase was dried over Na₂SO₄, filtered and concentrated in vacuo. The residue was purified by Prep-HPLC to afford tert-butyl 2-((S)-1-(4-fluorophenyl)-1,2,3,4-tetrahydroisoquinoline-2-carbonyl)-1,4-oxazepane-4-carboxylate.

LCMS: (M+H)⁺=455.1; purity=100% (214 nm); Retention time=1.77 min. Method C1

Step 2: To a round bottomed flask charged with a solution of HCl in Dioxane (4.0 M, 10 mL) was added tert-butyl 2-((S)-1-(4-fluorophenyl)-1,2,3,4-tetrahydroisoquinoline-2-carbonyl)-1,4-oxazepane-4-carboxylate (320 mg, 0.70 mmol) and the reaction mixture was stirred at room temperature for 1 h. The mixture was concentrated to give a residue which was purified by Prep-HPLC to give ((S)-1-(4-fluorophenyl)-3,4-dihydroisoquinolin-2(1H)-yl)(1,4-oxazepan-2-yl)methanone.

LCMS: (M+H)⁺=355.1; purity=100% (214 nm); Retention time=1.47 min. Method C1

The diastereomers were separated by chiral SFC eluting with C02/IPA (0.2% Methanol Ammonia)=50/50 over a Daicel® OZ column (20×250 mm, 10 μm) to give Compound 5091 and Compound 5090. Stereochemical assignment of (S) at 1 position of the tetrahydroisoquinoline is based on enantiomerically pure starting materials; the stereochemical configuration of the chiral center on the 7-membered ring is arbitrarily assigned.

Compound 5090: LCMS:(M+H)⁺=355.0 purity=100% (214 nm); Retention time=1.74 min. Method C1. Chiral SFC: CO₂/MeOH 0.2% MA=60/40 over Daicel® OZ column (4.6*100 mm, 3 um), retention time=2.009 min, 100% ee.

Compound 5091: LCMS: (M+H)⁺=355.0; purity=100% (214 nm); Retention time=1.73 min. Method C1. Chiral SFC: CO₂/MeOH 0.2% MA=60/40 over Daicel® OZ column (4.6*100 mm, 3 um), retention time=1.935 min, 100% ee.

### Example 60

### ((2R,4R,5S)-5-amino-4-fluorotetrahydro-2H-pyran-2-yl)((S)-1-(4-fluorophenyl)-3,4-dihydroisoquinolin-2(1H)-yl)methanone (COMPOUND 5075)

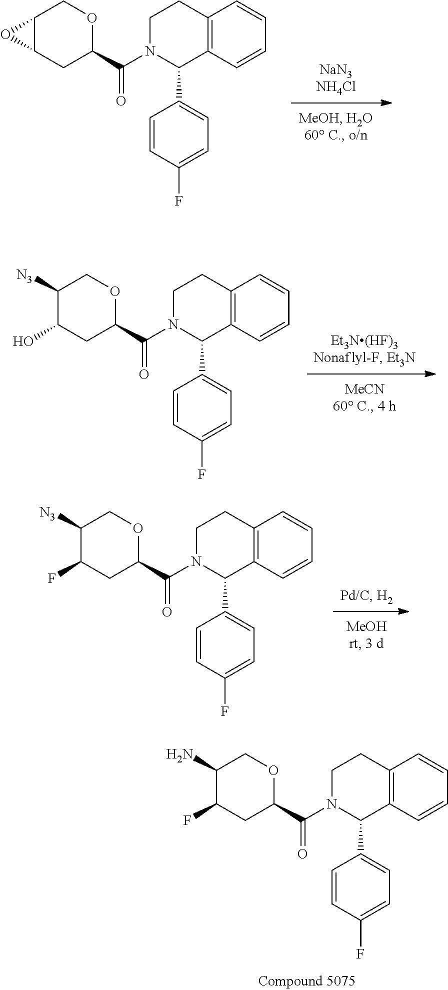

### Synthesis of ((2R,4S,5S)-5-azido-4-hydroxytetrahydro-2H-pyran-2-yl)((S)-1-(4-fluorophenyl)-3,4-dihydroisoquinolin-2(1H)-yl)methanone

Ammonium chloride (0.605 g, 11.32 mmol) and sodium azide (1.472 g, 22.64 mmol) were added to a solution of ((1R,4R,6S)-3,7-dioxabicyclo[4.1.0]heptan-4-yl)((S)-1-(4-fluorophenyl)-3,4-dihydroisoquinolin-2(1H)-yl)methanone (see COMPOUND 5070, 0.80 g, 2.264 mmol) in a mixture of methanol (100 mL) and water (12.5 mL). The reaction mixture was warmed to 60° C. and stirred overnight. After cooling to room temperature, the reaction mixture was diluted with water and extracted with ethyl acetate (2×). The combined organics were washed with brine, dried over Na₂SO₄, and evaporated under reduced pressure. The residue was purified by flash column chromatography (silica, 0 to 100% ethyl acetate in heptane) to give ((2R,4S,5S)-5-azido-4-hydroxytetrahydro-2H-pyran-2-yl)((S)-1-(4-fluorophenyl)-3,4-dihydroisoquinolin-2(1H)-yl)methanone.

LCMS: 92%, RT=2.04 min., (M+H)⁺=397 (method A).

### Synthesis of ((2R,4R,5S)-5-azido-4-fluorotetrahydro-2H-pyran-2-yl)((S)-1-(4-fluorophenyl)-3,4-dihydroisoquinolin-2(1H)-yl)methanone

Under argon atmosphere, triethylamine (0.174 mL, 1.249 mmol), triethylamine trihydrofluoride (0.068 mL, 0.416 mmol) and perfluoro-1-butanesulfonyl fluoride (0.075 mL, 0.416 mmol) were added to a solution of ((2R,4S,5S)-5-azido-4-hydroxytetrahydro-2H-pyran-2-yl)((S)-1-(4-fluorophenyl)-3,4-dihydroisoquinolin-2(1H)-yl)methanone (75 mg, 0.189 mmol) in acetonitrile (anhydrous, 2 mL). The reaction mixture was warmed to 60° C. and stirred for 2 hours. Then, the mixture was allowed to cool to room temperature and stirred overnight. Additional triethylamine (0.087 mL, 0.624 mmol), triethylamine trihydrofluoride (0.034 mL, 0.208 mmol) and perfluoro-1-butanesulfonyl fluoride (0.037 mL, 0.208 mmol) were added. The reaction mixture was warmed to 60° C. and stirred for 2 hours. The reaction mixture was poured into ice cold saturated aqueous NaHCO₃ solution and the mixture was extracted with ethyl acetate (twice). The combined organic layers were dried over Na₂SO₄ and evaporated under reduced pressure. The residue was purified by flash column chromatography (silica, 0 to 100% ethyl acetate in heptane) to give ((2R,4R,5S)-5-azido-4-fluorotetrahydro-2H-pyran-2-yl)((S)-1-(4-fluorophenyl)-3,4-dihydroisoquinolin-2(1H)-yl)methanone (50% pure, 50 mg).

LCMS: 91%, RT=2.19 min., (M+H)⁺=399 (method A). SFC: 50%, RT=2.10 min., (M+H)⁺=399 (method AD).

### Synthesis of ((2R,4R,5S)-5-amino-4-fluorotetrahydro-2H-pyran-2-yl)((S)-1-(4-fluorophenyl)-3,4-dihydroisoquinolin-2(1H)-yl)methanone (COMPOUND 5075)

A solution of ((2R,4R,5S)-5-azido-4-fluorotetrahydro-2H-pyran-2-yl)((S)-1-(4-fluorophenyl)-3,4-dihydroisoquinolin-2(1/H)-yl)methanone (50% pure, 50 mg, 0.063 mmol) in methanol (extra dry, 5 mL) was hydrogenated in the presence of palladium (10 wt % on carbon, containing 50% water, 26.7 mg, 0.013 mmol) and atmospheric hydrogen pressure. After 3 days, the reaction mixture was filtered and evaporated under reduced pressure. The residue was purified by basic preparative MPLC (Linear Gradient: t=0 min 5% A; t=1 min 5% A; t=2 min 20% A; t=17 min 60% A; t=18 min 100% A; t=23 min 100% A; detection: 220 nm) to give ((2R,4R,5S)-5-amino-4-fluorotetrahydro-2H-pyran-2-yl)((S)-1-(4-fluorophenyl)-3,4-dihydroisoquinolin-2(1/H)-yl)methanone (COMPOUND 5075).

LCMS: 97%, RT=1.22 min., (M+H)⁺=373 (method P). SFC: 92%, RT=3.20 min., (M+H)⁺=373 (method W).

### Example 61

### ((2S,4R,5R)-5-amino-4-methyltetrahydro-2H-pyran-2-yl)((S)-1-(4-fluorophenyl)-3,4-dihydroisoquinolin-2(1H)-yl)methanone (COMPOUND 5084) and ((2S,4S,5R)-5-amino-4-methyltetrahydro-2H-pyran-2-yl)((S)-1-(4-fluorophenyl)-3,4-dihydroisoquinolin-2(1H)-yl)methanone (COMPOUND 5076)

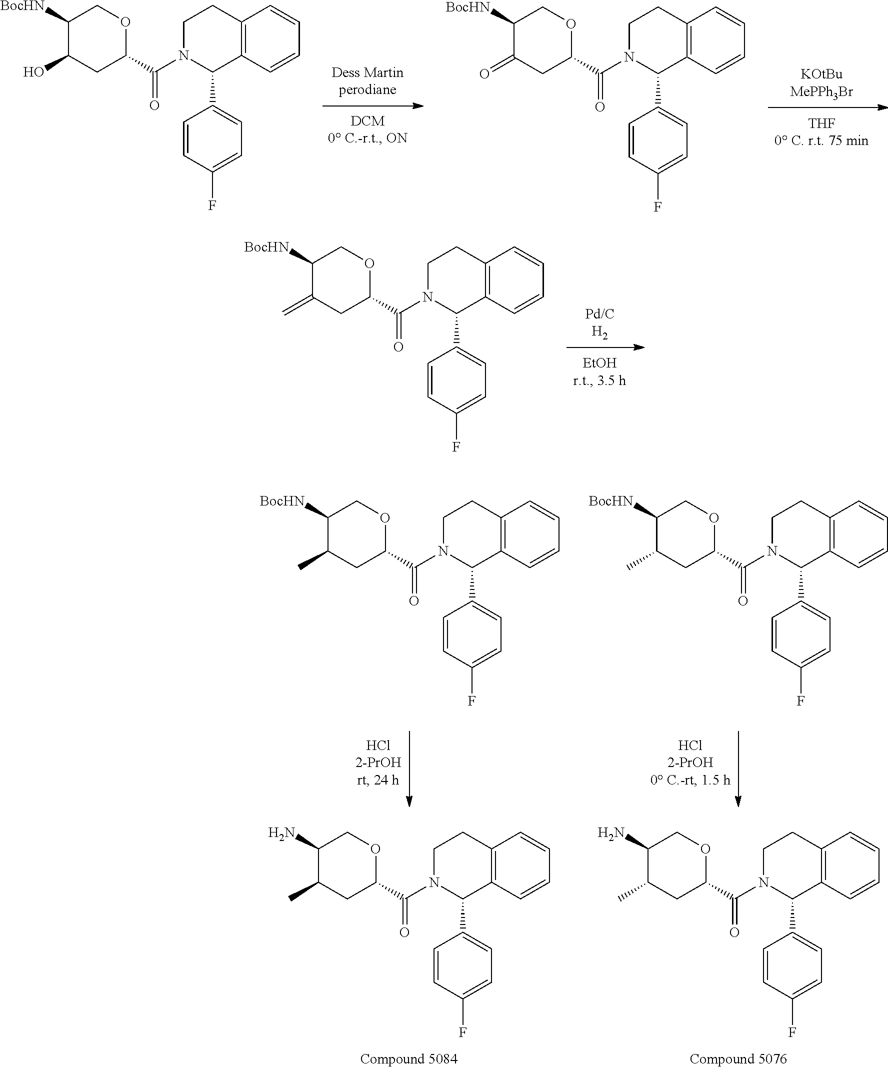

### Synthesis of tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate

At 0° C., Dess-Martin periodinane (191 mg, 0.450 mmol) was added to a solution of tert-butyl ((3S,4R,6S)-6-((S)-1-(4-fluorophenyl)-1,2,3,4-tetrahydroisoquinoline-2-carbonyl)-4-hydroxytetrahydro-2H-pyran-3-yl)carbamate (see COMPOUND 5053, 200 mg, 0.409 mmol) in dichloromethane (dry, 4.0 mL). The reaction mixture was stirred at room temperature overnight. Then, the mixture was diluted with a mixture of saturated aqueous NaHCO₃ and saturated aqueous Na₂S₂O₃ (1/1 (v/v), 5 mL) and stirred vigorously for 15 minutes. The mixture was further diluted with dichloromethane (10 mL) and a mixture of saturated aqueous NaHCO₃ and saturated aqueous Na₂S₂O₃ (1/1 (v/v), 5 mL) and the layers were separated. The aqueous layer was extracted with dichloromethane (5 mL). The combined organics were dried over Na₂SO₄ and evaporated under reduced pressure. The residue was purified by flash column chromatography (silica, 1 to 35% ethyl acetate in heptane) to give tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate.

LCMS: 83%, RT=2.16 min., (M+Na)⁺=491 and 16% hydrated product RT=2.03 min., (M+Na)⁺=509 (method A).

### Synthesis of tert-butyl ((3R,6S)-6-((S)-1-(4-fluorophenyl)-1,2,3,4-tetrahydroisoquinoline-2-carbonyl)-4-methylenetetrahydro-2H-pyran-3-yl)carbamate

At 0° C., potassium tert-butoxide (1.0 M solution in tert-butanol, 0.423 mL, 0.423 mmol) was added to a suspension of methyltriphenylphosphonium bromide (176 mg, 0.493 mmol) in tetrahydrofuran (dry, 2.4 mL). After 30 minutes, a solution of tert-butyl ((3S,6S)-6-((S)-1-(4-fluorophenyl)-1,2,3,4-tetrahydroisoquinoline-2-carbonyl)-4-oxotetrahydro-2H-pyran-3-yl)carbamate (165 mg, 0.352 mmol) in tetrahydrofuran (dry, 1.2 mL) was added dropwise and the mixture was stirred for 75 minutes. Then, the reaction mixture was filtered through Celite and washed with tetrahydrofuran. The filtrate was concentrated to dryness under reduced pressure and the residue was purified by flash column chromatography (silica, 10 to 45% ethyl acetate in heptane) to give tert-butyl ((3R,6S)-6-((S)-1-(4-fluorophenyl)-1,2,3,4-tetrahydroisoquinoline-2-carbonyl)-4-methylenetetrahydro-2H-pyran-3-yl)carbamate.

LCMS: 93%, RT=2.20 min., (M+H)⁺=467 (method A).

### Synthesis of tert-butyl ((3R,4R,6S)-6-((S)-1-(4-fluorophenyl)-1,2,3,4-tetrahydroisoquinoline-2-carbonyl)-4-methyltetrahydro-2H-pyran-3-yl)carbamate and tert-butyl ((3R,4S,6S)-6-((S)-1-(4-fluorophenyl)-1,2,3,4-tetrahydroisoquinoline-2-carbonyl)-4-methyltetrahydro-2H-pyran-3-yl)carbamate

A solution of ((3R,6S)-6-((S)-1-(4-fluorophenyl)-1,2,3,4-tetrahydroisoquinoline-2-carbonyl)-4-methylenetetrahydro-2H-pyran-3-yl)carbamate (133 mg, 0.285 mmol) in ethanol (2.0 mL) was hydrogenated in the presence of palladium (10 wt % on carbon, containing 50% water, 60.7 mg, 0.029 mmol) and atmospheric hydrogen pressure. After 3.5 hours, the reaction mixture was filtered through Celite and the filter cake was washed with methanol. The filtrate was evaporated under reduced pressure. The residue was purified by flash column chromatography (silica, 10 to 35% ethyl acetate in heptane) to give tert-butyl ((3R,4R,6S)-6-((S)-1-(4-fluorophenyl)-1,2,3,4-tetrahydroisoquinoline-2-carbonyl)-4-methyltetrahydro-2H-pyran-3-yl)carbamate as the first eluting isomer and tert-butyl ((3R,4S,6S)-6-((S)-1-(4-fluorophenyl)-1,2,3,4-tetrahydroisoquinoline-2-carbonyl)-4-methyltetrahydro-2H-pyran-3-yl)carbamate as the second eluting isomer.

First eluting isomer: LCMS: 99%, RT=2.26 min., (M+H)⁺=469 (method A).

Second eluting isomer: LCMS: 97%, RT=2.20 min., (M+H)⁺=469 (method A).

### Synthesis of ((2S,4R,5R)-5-amino-4-methyltetrahydro-2H-pyran-2-yl)((S)-1-(4-fluorophenyl)-3,4-dihydroisoquinolin-2(1H)-yl)methanone (COMPOUND 5084)

HCl (5-6 M in 2-propanol, 83 μL, 0.46 mmol) was added to a solution of tert-butyl ((3R,4R,6S)-6-((S)-1-(4-fluorophenyl)-1,2,3,4-tetrahydroisoquinoline-2-carbonyl)-4-methyltetrahydro-2H-pyran-3-yl)carbamate (9.0 mg, 0.019 mmol) in 2-propanol (0.2 mL) and stirred for 24 hours. The reaction mixture was diluted with dichloromethane (2 mL) and saturated aqueous Na₂CO₃ (1 mL). The layers were separated and the aqueous phase was extracted with dichloromethane (2×2 mL). The combined organics were dried over Na₂SO₄ and evaporated under reduced pressure. The residue was dissolved in methanol (1 mL) and brought onto an SCX-2 column (1 g) and eluted with methanol until neutral. Next, the column was eluted with ammonia in methanol (1 M). The basic fraction was concentrated to dryness under reduced pressure and lyophilised from a mixture of acetonitrile and water (1:1, 4 mL) to give ((2S,4R,5R)-5-amino-4-methyltetrahydro-2H-pyran-2-yl)((S)-1-(4-fluorophenyl)-3,4-dihydroisoquinolin-2(1H)-yl)methanone (COMPOUND 5084).

LCMS: 99%, RT=1.06 min., (M+H)⁺=369 (method P). SFC: RT=3.76 min., (M+H)⁺=369 (method W).

### Synthesis of ((2S,4S,5R)-5-amino-4-methyltetrahydro-2H-pyran-2-yl)((S)-1-(4-fluorophenyl)-3,4-dihydroisoquinolin-2(1H)-yl)methanone (COMPOUND 5076)

At 0° C., HCl (5-6 M in 2-propanol, 0.854 mL, 4.70 mmol) was added to tert-butyl ((3R,4S,6S)-6-((S)-1-(4-fluorophenyl)-1,2,3,4-tetrahydroisoquinoline-2-carbonyl)-4-methyltetrahydro-2H-pyran-3-yl)carbamate (44.0 mg, 0.094 mmol). After 5 minutes, the reaction mixture was allowed to warm to room temperature and stirred for 1.5 hours. The reaction mixture was diluted with dichloromethane (7 mL) and saturated aqueous Na₂CO₃ (4 mL). The layers were separated and the aqueous phase was extracted with dichloromethane (2×3 mL). The combined organics were dried over Na₂SO₄ and evaporated under reduced pressure. The residue was dissolved in methanol (1 mL) and brought onto an SCX-2 column (6 g) and eluted with methanol until neutral. Next, the column was eluted with ammonia in methanol (1 M). The basic fraction was concentrated to dryness under reduced pressure and lyophilised from a mixture of acetonitrile and water (1:1, 4 mL) to give ((2S,4S,5R)-5-amino-4-methyltetrahydro-2H-pyran-2-yl)((S)-1-(4-fluorophenyl)-3,4-dihydroisoquinolin-2(1H)-yl)methanone (COMPOUND 5076).

LCMS: 99%, RT=1.06 min., (M+H)⁺=369 (method P). SFC: RT=3.79 min., (M+H)⁺=369 (method W).

### Example 62

### ((2R,4S,5R)-4-amino-5-methoxytetrahydro-2H-pyran-2-yl)((S)-1-(4-fluorophenyl)-3,4-dihydroisoquinolin-2(1H)-yl)methanone (COMPOUND 5077)

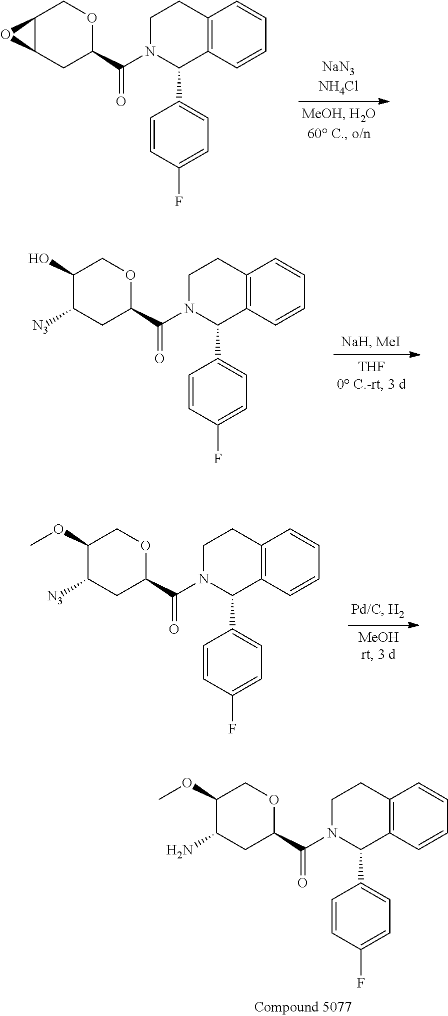 [SMILES: O=C([C@H]1C[C@H]2O[C@H]2CO1)N1CCc2ccccc2[C@@H]1c1ccc(F)cc1 ; [N-]=[N+]=N[C@H]1C[C@H](C(=O)N2CCc3ccccc3[C@@H]2c2ccc(F)cc2)OC[C@@H]1O ; CO[C@H]1CO[C@@H](C(=O)N2CCc3ccccc3[C@@H]2c2ccc(F)cc2)C[C@@H]1N=[N+]=[N-] ; CO[C@H]1CO[C@@H](C(=O)N2CCc3ccccc3[C@@H]2c2ccc(F)cc2)C[C@@H]1N]

### Synthesis of ((2R,4S,5R)-4-azido-5-hydroxytetrahydro-2H-pyran-2-yl)((S)-1-(4-fluorophenyl)-3,4-dihydroisoquinolin-2(1H)-yl)methanone

Ammonium chloride (0.103 g, 6.04 mmol) and sodium azide (0.786 g, 12.1 mmol) were added to a solution of ((1S,4R,6R)-3,7-dioxabicyclo[4.1.0]heptan-4-yl)((S)-1-(4-fluorophenyl)-3,4-dihydroisoquinolin-2(1H)-yl)methanone (see COMPOUND 5070, 0.427 g, 1.208 mmol) in a mixture of methanol (50 mL) and water (6.25 mL). The reaction mixture was warmed to 60° C. and stirred overnight. After cooling to room temperature, the reaction mixture was diluted with water and extracted with ethyl acetate (2×). The combined organics were washed with brine, dried over Na₂SO₄, and evaporated under reduced pressure. The residue was purified by flash column chromatography (silica, 0 to 100% ethyl acetate in heptane) to give ((2R,4S,5R)-4-azido-5-hydroxytetrahydro-2H-pyran-2-yl)((S)-1-(4-fluorophenyl)-3,4-dihydroisoquinolin-2(1H)-yl)methanone.

LCMS: 100%, RT=2.04 min., (M+H)⁺=397 (method A).

### Synthesis of ((2R,4S,5R)-4-azido-5-methoxytetrahydro-2H-pyran-2-yl)((S)-1-(4-fluorophenyl)-3,4-dihydroisoquinolin-2(1H)-yl)methanone

Under argon atmosphere, sodium hydride (60% dispersion in oil, 17.7 mg, 0.443 mmol) followed by iodomethane (22.1 μL, 0.354 mmol) were added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (70 mg, 0.177 mmol) in tetrahydrofuran (dry, 6 mL). The reaction mixture was allowed to warm to room temperature and stirred for 18 hours. The reaction mixture was diluted with aqueous KHSO₄ (0.1 M) and extracted with ethyl acetate (twice). The combined organic layers were dried over Na₂SO₄ and evaporated under reduced pressure. The residue was purified by flash column chromatography (silica, 0 to 100% ethyl acetate in heptane) to give ((2R,4S,5R)-4-azido-5-methoxytetrahydro-2H-pyran-2-yl)((S)-1-(4-fluorophenyl)-3,4-dihydroisoquinolin-2(1H)-yl)methanone.

LCMS: 99%, RT=2.18 min., (M+H)⁺=411 (method A).

### Synthesis of ((2R,4S,5R)-4-amino-5-methoxytetrahydro-2H-pyran-2-yl)((S)-1-(4-fluorophenyl)-3,4-dihydroisoquinolin-2(1H)-yl)methanone (COMPOUND 5077)

A solution of ((2R,4S,5R)-4-azido-5-methoxytetrahydro-2H-pyran-2-yl)((S)-1-(4-fluorophenyl)-3,4-dihydroisoquinolin-2(1H)-yl)methanone (51.7 mg, 0.126 mmol) in methanol (extra dry, 5 mL) was hydrogenated in the presence of palladium (10 wt % on carbon, containing 50% water, 26.8 mg, 0.013 mmol) and atmospheric hydrogen pressure. After 3 days, the reaction mixture was filtered and evaporated under reduced pressure. The residue was purified by basic preparative MPLC (Linear Gradient: t=0 min 5% A; t=1 min 5% A; t=2 min 20% A; t=17 min 60% A; t=18 min 100% A; t=23 min 100% A; detection: 220 nm) to give ((2R,4S,5R)-4-amino-5-methoxytetrahydro-2H-pyran-2-yl)((S)-1-(4-fluorophenyl)-3,4-dihydroisoquinolin-2(1H)-yl)methanone (COMPOUND 5077).

LCMS: 99%, RT=1.25 min., (M+H)⁺=385 (method P).

### Example 63

### ((2R,4S,5R)-4-amino-5-ethoxytetrahydro-2H-pyran-2-yl)((S)-1-(4-fluorophenyl)-3,4-dihydroisoquinolin-2(1H)-yl)methanone (COMPOUND 5078)

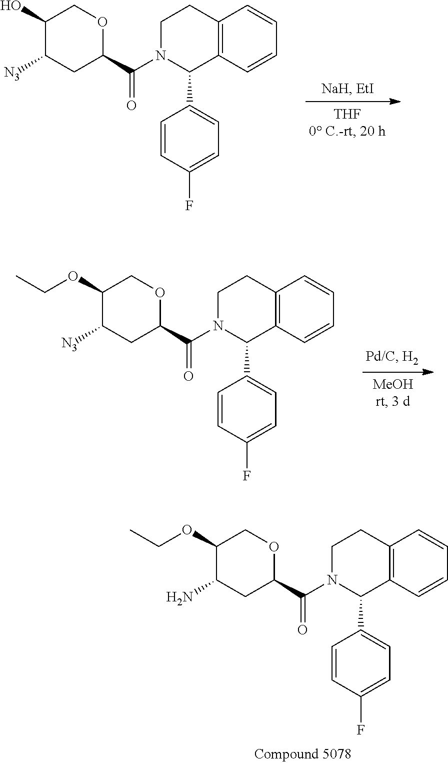 [SMILES: [N-]=[N+]=N[C@H]1C[C@H](C(=O)N2CCc3ccccc3[C@@H]2c2ccc(F)cc2)OC[C@@H]1O ; CCO[C@H]1CO[C@@H](C(=O)N2CCc3ccccc3[C@@H]2c2ccc(F)cc2)C[C@@H]1N=[N+]=[N-] ; CCO[C@H]1CO[C@@H](C(=O)N2CCc3ccccc3[C@@H]2c2ccc(F)cc2)C[C@@H]1N]

### Synthesis of ((2R,4S,5R)-4-azido-5-ethoxytetrahydro-2H-pyran-2-yl)((S)-1-(4-fluorophenyl)-3,4-dihydroisoquinolin-2(1H)-yl)methanone

Under argon atmosphere, sodium hydride (60% dispersion in oil, 12.6 mg, 0.315 mmol) followed by iodoethane (20 μL, 0.252 mmol) were added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see COMPOUND 5077, 50 mg, 0.126 mmol) in tetrahydrofuran (dry, 4 mL). The reaction mixture was allowed to warm to room temperature and stirred for 18 hours. Then, another portion of sodium hydride (60% dispersion in oil, 12.6 mg, 0.315 mmol) and iodoethane (20 μL, 0.252 mmol) were added and stirring was continued for another 2 hours. The reaction mixture was diluted with aqueous KHSO₄ (0.1 M) and extracted with ethyl acetate (twice). The combined organic layers were dried over Na₂SO₄ and evaporated under reduced pressure. The residue was purified by flash column chromatography (silica, 0 to 100% ethyl acetate in heptane) to give ((2R,4S,5R)-4-azido-5-ethoxytetrahydro-2H-pyran-2-yl)((S)-1-(4-fluorophenyl)-3,4-dihydroisoquinolin-2(1H)-yl)methanone.

LCMS: 98%, RT=2.25 min., (M+H)⁺=425 (method A).

### Synthesis of ((2R,4S,5R)-4-amino-5-ethoxytetrahydro-2H-pyran-2-yl)((S)-1-(4-fluorophenyl)-3,4-dihydroisoquinolin-2(1H)-yl)methanone (COMPOUND 5078)

A solution of ((2R,4S,5R)-4-azido-5-ethoxytetrahydro-2H-pyran-2-yl)((S)-1-(4-fluorophenyl)-3,4-dihydroisoquinolin-2(1H)-yl)methanone (53 mg, 0.126 mmol) in methanol (extra dry, 5 mL) was hydrogenated in the presence of palladium (10 wt % on carbon, containing 50% water, 26.8 mg, 0.013 mmol) and atmospheric hydrogen pressure. After 3 days, the reaction mixture was filtered and evaporated under reduced pressure. The residue was purified by basic preparative MPLC (Linear Gradient: t=0 min 5% A; t=1 min 5% A; t=2 min 20% A; t=17 min 60% A; t=18 min 100% A; t=23 min 100% A; detection: 220 nm) to give ((2R,4S,5R)-4-amino-5-ethoxytetrahydro-2H-pyran-2-yl)((S)-1-(4-fluorophenyl)-3,4-dihydroisoquinolin-2(1H)-yl)methanone (COMPOUND 5078).

LCMS: 99%, RT=1.21 min., (M+H)⁺=399 (method P).

### Example 64

### (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one (COMPOUND 5079)

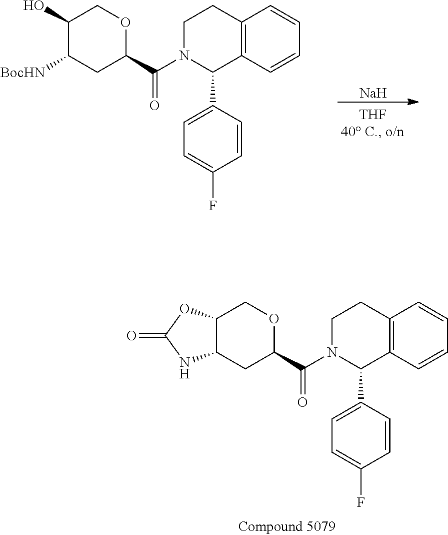

Sodium hydride (60 wt % dispersion in mineral oil, 25.5 mg, 0.638 mmol) was added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 300 mg, 0.638 mmol) in tetrahydrofuran (dry, 15 mL) and the mixture was stirred at 40° C. overnight. The mixture was diluted with dichloromethane (25 mL) and saturated aqueous NH₄Cl (20 mL). The layers were separated and the aqueous phase was extracted with dichloromethane (2×25 mL). The combined organics were dried over Na₂SO₄ and evaporated under reduced pressure. The residue was purified by flash column chromatography (silica, 40 to 100% ethyl acetate in heptane) to give (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one.

LCMS: 100%, RT=1.51 min., (M+H)⁺=397 (method P).

### Example 65

### ((2R,4S,5R)-5-amino-4-methoxytetrahydro-2H-pyran-2-yl)((S)-1-(4-fluorophenyl)-3,4-dihydroisoquinolin-2(1H)-yl)methanone (COMPOUND 5082)

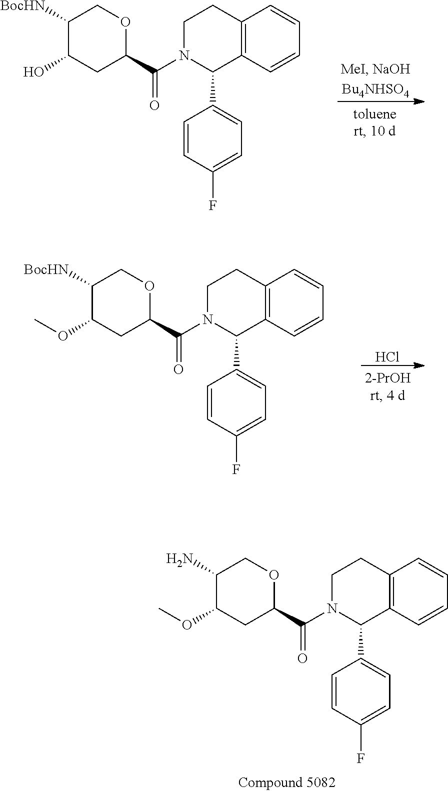

### Synthesis of tert-butyl ((3R,4S,6R)-6-((S)-1-(4-fluorophenyl)-1,2,3,4-tetrahydroisoquinoline-2-carbonyl)-4-methoxytetrahydro-2H-pyran-3-yl)carbamate

Tetrabutylammonium hydrogen sulfate (7.3 mg, 0.022 mmol) followed by aqueous NaOH (50 wt %, 11.7 μL, 0.223 mmol) and methyl iodide (21 μL, 0.335 mmol) were added to a solution of tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (see Compound 5052, 35 mg, 0.074 mmol) in toluene (3 mL). After stirring for 3 days, additional amounts of aqueous NaOH (50 wt %, 11.7 μL, 0.223 mmol) and methyl iodide (21 μL, 0.335 mmol) were added and stirring was continued for 7 days. The reaction mixture was diluted with dichloromethane (5 mL) and saturated aqueous NaHCO₃ (5 mL). The layers were separated using a phase separator and the organic filtrate was evaporated under reduced pressure. The residue was purified by flash column chromatography (silica, 0 to 50% ethyl acetate in heptane) to give tert-butyl ((3R,4S,6R)-6-((S)-1-(4-fluorophenyl)-1,2,3,4-tetrahydroisoquinoline-2-carbonyl)-4-methoxytetrahydro-2H-pyran-3-yl)carbamate.

LCMS: 99%, RT=2.24 min., (M+H)⁺=485 (method A).

### Synthesis of ((2R,4S,5R)-5-amino-4-methoxytetrahydro-2H-pyran-2-yl)((S)-1-(4-fluorophenyl)-3,4-dihydroisoquinolin-2(1H)-yl)methanone (COMPOUND 5082)

HCl (5-6 M in 2-propanol, 0.2 mL, 1.1 mmol) was added to a solution of tert-butyl ((3R,4S,6R)-6-((S)-1-(4-fluorophenyl)-1,2,3,4-tetrahydroisoquinoline-2-carbonyl)-4-methoxytetrahydro-2H-pyran-3-yl)carbamate (34 mg, 0.070 mmol) in 2-propanol (2.0 mL). After 3 days, additional HCl (5-6 M in 2-propanol, 0.2 mL, 1.1 mmol) was added and stirring was continued for 1 more day. The reaction mixture was diluted with dichloromethane (10 mL) and neutralised with saturated aqueous NaHCO₃. The layers were separated and the organic phase was washed with brine, dried over Na₂SO₄, and evaporated under reduced pressure. The residue was dissolved in methanol (1 mL) and brought onto an SCX-2 column (1 g) and eluted with methanol until neutral. Next, the column was eluted with ammonia in methanol (1.5 M). The basic fraction was concentrated to dryness under reduced pressure. The residue was lyophilised from a mixture of acetonitrile and water (1:3, 3 mL) to give ((2R,4S,5R)-5-amino-4-methoxytetrahydro-2H-pyran-2-yl)((S)-1-(4-fluorophenyl)-3,4-dihydroisoquinolin-2(1H)-yl)methanone (COMPOUND 5082).

LCMS: 99%, RT=1.05 min., (M+H)⁺=385 (method P).

### Example 66

### ((2S,4S,5R)-4-amino-5-hydroxytetrahydro-2H-pyran-2-yl)((S)-1-(4-fluorophenyl)-3,4-dihydroisoquinolin-2(1H)-yl)methanone (COMPOUND 5083)

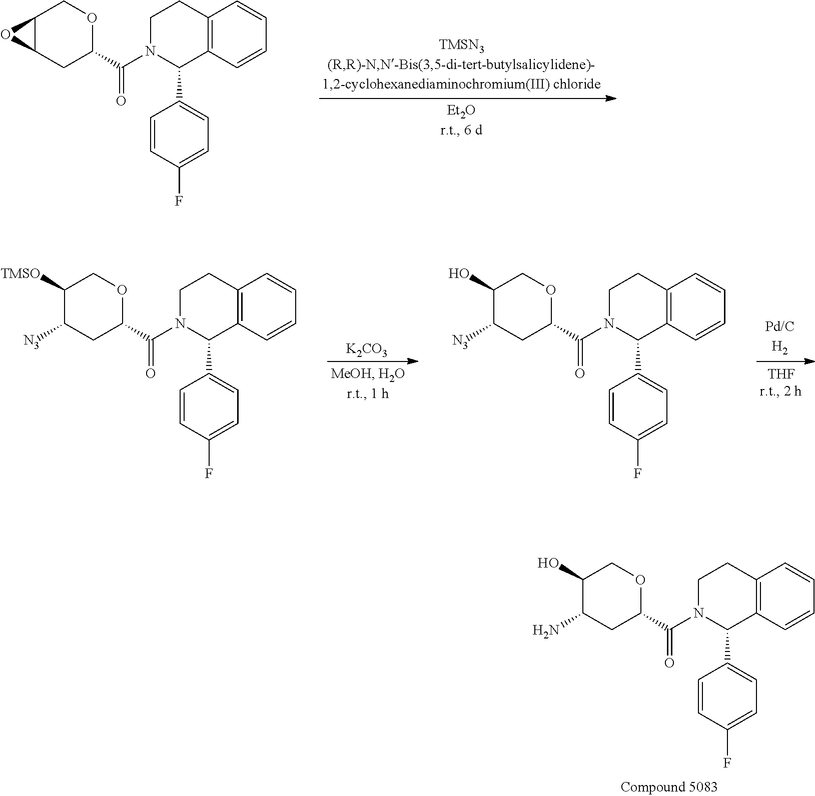

### Synthesis of ((2S,4S,5R)-4-azido-5-((trimethylsilyl)oxy)tetrahydro-2H-pyran-2-yl)((S)-1-(4-fluorophenyl)-3,4-dihydroisoquinolin-2(1H)-yl)methanone

Under nitrogen atmosphere, ((1S,4S,6R)-3,7-dioxabicyclo[4.1.0]heptan-4-yl)((S)-1-(4-fluorophenyl)-3,4-dihydroisoquinolin-2(1H)-yl)methanone (See Compound 5072, 305 mg, 0.863 mmol was added to a dark red suspension of (R,R)-N,N′-bis(3,5-di-tert-butylsalicylidene)-1,2-cyclohexanediaminochromium(III) chloride (10.91 mg, 0.017 mmol) in diethyl ether (dry, 1.2 mL). The resulting thick orange suspension was stirred for 15 minutes after which trimethylsilyl azide (0.127 mL, 0.906 mmol) was added. The reaction mixture was stirred overnight after which another portion of trimethylsilyl azide (0.089 mL, 0.636 mmol) was added. Stirring was continued for 5 days. The reaction mixture was concentrated under reduced pressure at room temperature. The residue was purified by flash column chromatography (silica, 6 to 16% ethyl acetate in heptane) to give ((2S,4S,5R)-4-azido-5-((trimethylsilyl)oxy)tetrahydro-2H-pyran-2-yl)((S)-1-(4-fluorophenyl)-3,4-dihydroisoquinolin-2(1H)-yl)methanone.

LCMS: 62%, RT=2.34 min., (M+H)⁺=469 and 38% product-TMS, RT=2.00 min., (M+H)⁺=397 (method A).

### Synthesis of ((2S,4S,5R)-4-azido-5-hydroxytetrahydro-2H-pyran-2-yl)((S)-1-(4-fluorophenyl)-3,4-dihydroisoquinolin-2(1H)-yl)methanone

Potassium carbonate (80 mg, 0.578 mmol) was added to a solution of ((2S,4S,5R)-4-azido-5-((trimethylsilyl)oxy)tetrahydro-2H-pyran-2-yl)((S)-1-(4-fluorophenyl)-3,4-dihydroisoquinolin-2(1H)-yl)methanone (91.5 mg, 0.193 mmol) in a mixture of methanol (3.0 mL) and water (0.375 mL). After stirring for 1 hour, the reaction mixture was diluted with dichloromethane (30 mL) and washed with brine (10 mL). The aqueous layer was extracted with dichloromethane (10 mL) and the combined organic layers were dried over Na₂SO₄ and evaporated under reduced pressure. The residue was purified by flash column chromatography (silica, 15 to 50% ethyl acetate in heptane) to give ((2S,4S,5R)-4-azido-5-hydroxytetrahydro-2H-pyran-2-yl)((S)-1-(4-fluorophenyl)-3,4-dihydroisoquinolin-2(1H)-yl)methanone.

LCMS: 100%, RT=2.03 min., (M+H)⁺=397 (method A).

### Synthesis of ((2S,4S,5R)-4-amino-5-hydroxytetrahydro-2H-pyran-2-yl)((S)-1-(4-fluorophenyl)-3,4-dihydroisoquinolin-2(1H)-yl)methanone (COMPOUND 5083)

A solution of ((2S,4S,5R)-4-azido-5-hydroxytetrahydro-2H-pyran-2-yl)((S)-1-(4-fluorophenyl)-3,4-dihydroisoquinolin-2(1H)-yl)methanone (27.0 mg, 0.067 mmol) in tetrahydrofuran (500 μL) was hydrogenated in the presence of palladium (10 wt % on carbon, containing 50% water, 14.35 mg, 6.74 μmol) and atmospheric hydrogen pressure. After 2 hours, the reaction mixture was filtered through Celite and the filter cake was washed with tetrahydrofuran. The filtrate was evaporated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure and lyophilised from a mixture of acetonitrile and water (1:1, 4 mL) to give ((2S,4S,5R)-4-amino-5-hydroxytetrahydro-2H-pyran-2-yl)((S)-1-(4-fluorophenyl)-3,4-dihydroisoquinolin-2(1/H)-yl)methanone (COMPOUND 5083).

LCMS: 98%, RT=1.01 min., (M+H)⁺=371 (method P). SFC: 100%, RT=3.77 min., (M+H)⁺=371 (method AD).

### Example 67

### ((2S,4S,5R)-4-amino-5-hydroxytetrahydro-2H-pyran-2-yl)((S)-1-(4-fluorophenyl)-3,4-dihydroisoquinolin-2(1H)-yl)methanone (COMPOUND 5085)

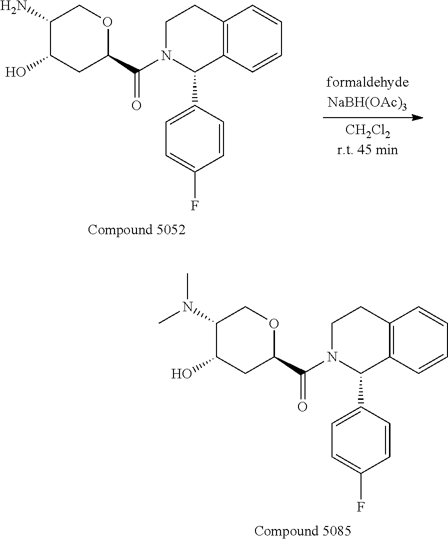 [SMILES: N[C@@H]1CO[C@@H](C(=O)N2CCc3ccccc3[C@@H]2c2ccc(F)cc2)C[C@@H]1O ; CN(C)[C@@H]1CO[C@@H](C(=O)N2CCc3ccccc3[C@@H]2c2ccc(F)cc2)C[C@@H]1O]

### Synthesis of ((2R,4S,5R)-5-(dimethylamino)-4-hydroxytetrahydro-2H-pyran-2-yl)((S)-1-(4-fluorophenyl)-3,4-dihydroisoquinolin-2(1H)-yl)methanone (COMPOUND 5085)

Formaldehyde (37 wt % solution in water stabilized with 10-15% methanol, 8.5 μL, 0.113 mmol) followed by sodium triacetoxyborohydride (34.3 mg, 0.162 mmol) were added to a solution of ((2R,4S,5R)-5-amino-4-hydroxytetrahydro-2H-pyran-2-yl)((S)-1-(4-fluorophenyl)-3,4-dihydroisoquinolin-2(1/H)-yl)methanone ((See Compound 5052, 60 mg, 0.162 mmol) in dichloromethane (1 mL). After 30 minutes, another portion of formaldehyde (37 wt % solution in water stabilized with 10-15% methanol, 0.037 mL, 0.486 mmol) and sodium triacetoxyborohydride (103 mg, 0.486 mmol) were added. After 15 minutes, the reaction mixture was partitioned between dichloromethane (5 mL) and saturated aqueous NaHCO₃ (5 mL). The organic layer was separated, washed with brine (5 mL), dried over Na₂SO₄, and evaporated under reduced pressure. The residue was dissolved in methanol (2 mL), loaded onto an SCX-2 column and eluted with methanol until neutral. Next, the column was eluted with ammonia in methanol (1.5 M). The basic fraction was concentrated to dryness under reduced pressure and lyophilised from a mixture of acetonitrile and water (1:2, 3 mL) to give ((2R,4S,5R)-5-(dimethylamino)-4-hydroxytetrahydro-2H-pyran-2-yl)((S)-1-(4-fluorophenyl)-3,4-dihydroisoquinolin-2(1H)-yl)methanone (COMPOUND 5085).

LCMS: 99%, RT=1.01 min., (M+H)⁺=399 (method P).

### Example 68

### ((2S,4R,5S)-5-amino-4-fluorotetrahydro-2H-pyran-2-yl)((S)-1-(4-fluorophenyl)-3,4-dihydroisoquinolin-2(1H)-yl)methanone (COMPOUND 5086)

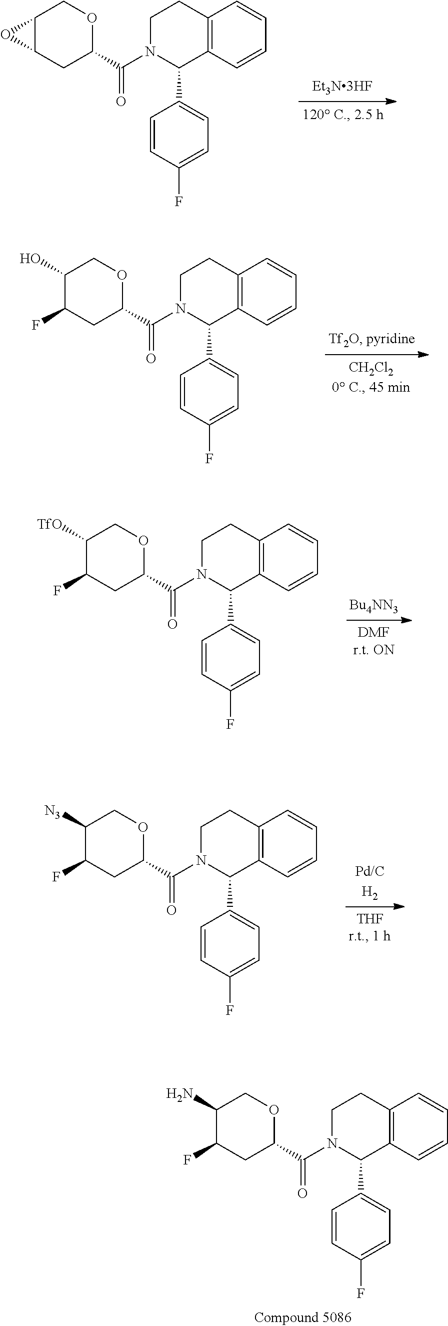 [SMILES: O=C([C@@H]1C[C@@H]2O[C@@H]2CO1)N1CCc2ccccc2[C@@H]1c1ccc(F)cc1 ; O=C([C@@H]1C[C@@H](F)[C@H](O)CO1)N1CCc2ccccc2[C@@H]1c1ccc(F)cc1 ; O=C([C@@H]1C[C@@H](F)[C@H](OS(=O)(=O)C(F)(F)F)CO1)N1CCc2ccccc2[C@@H]1c1ccc(F)cc1 ; [N-]=[N+]=N[C@H]1CO[C@H](C(=O)N2CCc3ccccc3[C@@H]2c2ccc(F)cc2)C[C@H]1F ; N[C@H]1CO[C@H](C(=O)N2CCc3ccccc3[C@@H]2c2ccc(F)cc2)C[C@H]1F]

### Synthesis of ((2S,4R,5R)-4-fluoro-5-hydroxytetrahydro-2H-pyran-2-yl)((S)-1-(4-fluorophenyl)-3,4-dihydroisoquinolin-2(1H)-yl)methanone

In a microwave vial, triethylamine trihydrofluoride (363 μL, 2.228 mmol) was added to ((1R,4S,6S)-3,7-dioxabicyclo[4.1.0]heptan-4-yl)((S)-1-(4-fluorophenyl)-3,4-dihydroisoquinolin-2(1H)-yl)methanone (See Compound 5072, 175 mg, 0.495 mmol). The vial was capped and placed into a pre-heated sand bath of 120° C. After 2.5 hours, the reaction mixture was diluted with ice-cold water (ca 1 mL) and dichloromethane (2 mL), followed by the slow addition of saturated aqueous NaHCO₃ (4 mL). The mixture was further diluted with dichloromethane (10 mL) and saturated aqueous NaHCO₃ (2 mL) and the layers were separated. The aqueous layer was extracted with dichloromethane (10 mL) and the combined organic layers were dried over Na₂SO₄ and evaporated under reduced pressure. The residue was purified by flash column chromatography (silica, 15 to 55% ethyl acetate in heptane) and preparative chiral SFC (method Y) to give ((2S,4R,5R)-4-fluoro-5-hydroxytetrahydro-2H-pyran-2-yl)((S)-1-(4-fluorophenyl)-3,4-dihydroisoquinolin-2(1H)-yl)methanone.

LCMS: 99%, RT=2.01 min., (M+H)⁺=374 (method A).

### Synthesis of (3R,4R,6S)-4-fluoro-6-((S)-1-(4-fluorophenyl)-1,2,3,4-tetrahydroisoquinoline-2-carbonyl)tetrahydro-2H-pyran-3-yl trifluoromethanesulfonate

Under argon atmosphere at 0° C., triflic anhydride (36.7 μL, 0.221 mmol) was added dropwise to a solution of ((2S,4R,5R)-4-fluoro-5-hydroxytetrahydro-2H-pyran-2-yl)((S)-1-(4-fluorophenyl)-3,4-dihydroisoquinolin-2(1H)-yl)methanone (55.0 mg, 0.147 mmol) and pyridine (35.7 μL, 0.442 mmol) in dichloromethane (dry, 750 μL). After 45 minutes, the reaction mixture was diluted with dichloromethane (6 mL) and washed with aqueous citric acid (0.5 M, 2 mL), saturated aqueous NaHCO₃ (2 mL), and brine (2 mL). The organic layer was dried over Na₂SO₄ and evaporated under reduced pressure to give (3R,4R,6S)-4-fluoro-6-((S)-1-(4-fluorophenyl)-1,2,3,4-tetrahydroisoquinoline-2-carbonyl)tetrahydro-2H-pyran-3-yl trifluoromethanesulfonate (76 mg) as an orange solid which was used as such.

LCMS: 99%, RT=2.28 min., (M+H)⁺=506 (method A).

### Synthesis of ((2S,4R,5S)-5-azido-4-fluorotetrahydro-2H-pyran-2-yl)((S)-1-(4-fluorophenyl)-3,4-dihydroisoquinolin-2(1H)-yl)methanone

Under argon atmosphere, tetrabutylammonium azide (46.1 mg, 0.162 mmol) was added to a solution of (3R,4R,6S)-4-fluoro-6-((S)-1-(4-fluorophenyl)-1,2,3,4-tetrahydroisoquinoline-2-carbonyl)tetrahydro-2H-pyran-3-yl trifluoromethanesulfonate (74.5 mg, 0.147 mmol) in N,N-dimethylformamide (dry, 750 μL) and stirred overnight. The reaction mixture was diluted with ethyl acetate (10 mL) and washed with a mixture of saturated aqueous NaHCO₃ and brine (1:1, 4 mL) and then with brine (3×2 mL). The organic layer was dried over Na₂SO₄ and evaporated under reduced pressure. The residue was purified by flash column chromatography (silica, 1 to 30% ethyl acetate in heptane) to give ((2S,4R,5S)-5-azido-4-fluorotetrahydro-2H-pyran-2-yl)((S)-1-(4-fluorophenyl)-3,4-dihydroisoquinolin-2(1H)-yl)methanone.

LCMS: 100%, RT=2.16 min., (M+H)⁺=399 (method A).

### Synthesis of ((2S,4R,5S)-5-amino-4-fluorotetrahydro-2H-pyran-2-yl)((S)-1-(4-fluorophenyl)-3,4-dihydroisoquinolin-2(1H)-yl)methanone (COMPOUND 5086)

A solution of ((2S,4R,5S)-5-azido-4-fluorotetrahydro-2H-pyran-2-yl)((S)-1-(4-fluorophenyl)-3,4-dihydroisoquinolin-2(1H)-yl)methanone (46 mg, 0.115 mmol) in tetrahydrofuran (1 mL) was hydrogenated in the presence of palladium (10 wt % on carbon, containing 50% water, 24.5 mg, 0.011 mmol) and atmospheric hydrogen pressure. After 1 hour, the reaction mixture was filtered through Celite and the filter cake was washed with tetrahydrofuran. The filtrate was evaporated under reduced pressure. The residue was dissolved in methanol (1 mL) and brought onto an SCX-2 column (1 g) and eluted with methanol until neutral. Next, the column was eluted with ammonia in methanol (1 M). The basic fraction was concentrated to dryness under reduced pressure and lyophilised from a mixture of acetonitrile and water to give ((2S,4R,5S)-5-amino-4-fluorotetrahydro-2H-pyran-2-yl)((S)-1-(4-fluorophenyl)-3,4-dihydroisoquinolin-2(1H)-yl)methanone (COMPOUND 5086).

LCMS: 99%, RT=1.04 min., (M+H)⁺=373 (method P). SFC: RT=3.38 min., (M+H)⁺=373 (method AD).

### Example 69

### ((2R,4S,5S)-4-amino-5-fluorotetrahydro-2H-pyran-2-yl)((S)-1-(4-fluorophenyl)-3,4-dihydroisoquinolin-2(1H)-yl)methanone (COMPOUND 5087)

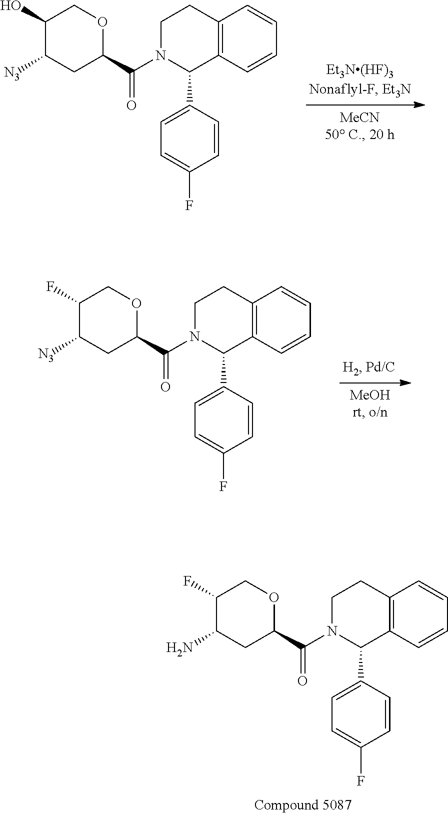 [SMILES: [N-]=[N+]=N[C@H]1C[C@H](C(=O)N2CCc3ccccc3[C@@H]2c2ccc(F)cc2)OC[C@@H]1O ; [N-]=[N+]=N[C@H]1C[C@H](C(=O)N2CCc3ccccc3[C@@H]2c2ccc(F)cc2)OC[C@H]1F ; N[C@H]1C[C@H](C(=O)N2CCc3ccccc3[C@@H]2c2ccc(F)cc2)OC[C@H]1F]

### Synthesis of ((2R,4S,5S)-4-azido-5-fluorotetrahydro-2H-pyran-2-yl)((S)-1-(4-fluorophenyl)-3,4-dihydroisoquinolin-2(1H)-yl)methanone

Under argon atmosphere, triethylamine (0.116 mL, 0.832 mmol), triethylamine trihydrofluoride (0.045 mL, 0.277 mmol) and perfluoro-1-butanesulfonyl fluoride (0.050 mL, 0.277 mmol) were added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 50 mg, 0.126 mmol) in acetonitrile (anhydrous, 2 mL). The reaction mixture was warmed to 50° C. and stirred for 18 hours. Then, the mixture was cooled to room temperature and additional triethylamine (0.058 mL, 0.416 mmol), triethylamine trihydrofluoride (0.023 mL, 0.139 mmol) and perfluoro-1-butanesulfonyl fluoride (0.025 mL, 0.139 mmol) were added. The reaction mixture was warmed to 50° C. and stirred for 2 hours. The reaction mixture was poured into ice cold saturated aqueous NaHCO₃ solution and the mixture was extracted with ethyl acetate (twice). The combined organic layers were dried over Na₂SO₄ and evaporated under reduced pressure. The residue was purified by flash column chromatography (silica, 0 to 100% ethyl acetate in heptane) to give ((2R,4S,5S)-4-azido-5-fluorotetrahydro-2H-pyran-2-yl)((S)-1-(4-fluorophenyl)-3,4-dihydroisoquinolin-2(1H)-yl)methanone.

LCMS: 99%, RT=2.20 min., (M+H)⁺=399 (method A).

### Synthesis of ((2R,4S,5S)-4-amino-5-fluorotetrahydro-2H-pyran-2-yl)((S)-1-(4-fluorophenyl)-3,4-dihydroisoquinolin-2(1H)-yl)methanone (COMPOUND 5087)

A solution of ((2R,4S,5S)-4-azido-5-fluorotetrahydro-2H-pyran-2-yl)((S)-1-(4-fluorophenyl)-3,4-dihydroisoquinolin-2(1H)-yl)methanone (38.7 mg, 0.063 mmol) in methanol (extra dry, 4 mL) was hydrogenated in the presence of palladium (10 wt % on carbon, containing 50% water, 25.1 mg, 0.012 mmol) and atmospheric hydrogen pressure. After stirring overnight, the reaction mixture was filtered and evaporated under reduced pressure. The residue was purified by preparative LCMS (method BU)

to give ((2R,4S,5S)-4-amino-5-fluorotetrahydro-2H-pyran-2-yl)((S)-1-(4-fluorophenyl)-3,4-dihydroisoquinolin-2(1H)-yl)methanone (COMPOUND 5087).

LCMS: 99%, RT=1.57 min., (M+H)⁺=373 (method Q).

### Example 70

### ((2R,4S,5R)-5-amino-4-ethoxytetrahydro-2H-pyran-2-yl)((S)-1-(4-fluorophenyl)-3,4-dihydroisoquinolin-2(1H)-yl)methanone (COMPOUND 5088)

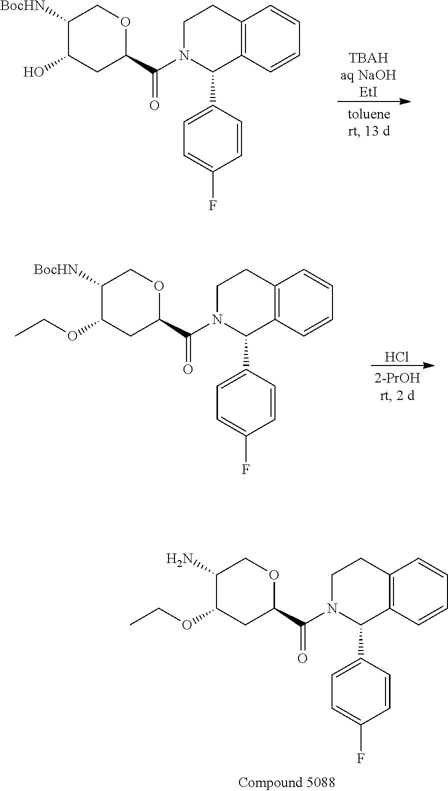 [SMILES: CC(C)(C)OC(=O)N[C@@H]1CO[C@@H](C(=O)N2CCc3ccccc3[C@@H]2c2ccc(F)cc2)C[C@@H]1O ; "TBAHaq NaOHEtI" ; CCO[C@H]1C[C@H](C(=O)N2CCc3ccccc3[C@@H]2c2ccc(F)cc2)OC[C@H]1NC(=O)OC(C)(C)C ; Cl ; CCO[C@H]1C[C@H](C(=O)N2CCc3ccccc3[C@@H]2c2ccc(F)cc2)OC[C@H]1N]

### Synthesis of tert-butyl ((3R,4S,6R)-4-ethoxy-6-((S)-1-(4-fluorophenyl)-1,2,3,4-tetrahydroisoquinoline-2-carbonyl)tetrahydro-2H-pyran-3-yl)carbamate

Tetrabutylammonium hydrogen sulfate (10.5 mg, 0.031 mmol) followed by aqueous NaOH (50 wt %, 5.6 μL, 0.106 mmol) and ethyl iodide (31 μL, 0.383 mmol) were added to a solution of tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (50 mg, 0.106 mmol) in toluene (4 mL). After stirring for 3 days, additional amounts of aqueous NaOH (50 wt %, 5.6 μL, 0.106 mmol) and ethyl iodide (31 μL, 0.383 mmol) were added and after another 5 days: aqueous NaOH (50 wt %, 22.4 μL, 0.425 mmol) and ethyl iodide (92 μL, 1.15 mmol). Stirring was continued for another 5 days, after which the reaction mixture was diluted with water (10 mL) and dichloromethane (15 mL). The layers were separated using a phase-separator and the organic filtrate was evaporated under reduced pressure. The residue was purified by flash column chromatography (silica, 0 to 50% ethyl acetate in heptane) to give tert-butyl ((3R,4S,6R)-4-ethoxy-6-((S)-1-(4-fluorophenyl)-1,2,3,4-tetrahydroisoquinoline-2-carbonyl)tetrahydro-2H-pyran-3-yl)carbamate.

LCMS: 99%, RT=2.27 min., (M+Na)⁺=521 (method A).

### Synthesis of ((2R,4S,5R)-5-amino-4-ethoxytetrahydro-2H-pyran-2-yl)((S)-1-(4-fluorophenyl)-3,4-dihydroisoquinolin-2(1H)-yl)methanone (COMPOUND 5088)

HCl (5-6 M in 2-propanol, 0.167 mL, 0.919 mmol) was added to a solution of tert-butyl ((3R,4S,6R)-4-ethoxy-6-((S)-1-(4-fluorophenyl)-1,2,3,4-tetrahydroisoquinoline-2-carbonyl)tetrahydro-2H-pyran-3-yl)carbamate (38 mg, 0.076 mmol) in 2-propanol (1 mL) and stirred overnight. An additional portion of HCl (5-6 M in 2-propanol, 0.167 mL, 0.919 mmol) was added and stirring was continued for one more day. The reaction mixture was diluted with dichloromethane (5 mL) and saturated aqueous NaHCO₃ (5 mL). The layers were separated and the aqueous phase was extracted with dichloromethane (3×10 mL). The combined organics were dried over Na₂SO₄ and evaporated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure and lyophilised from a mixture of acetonitrile and water (1:1, 4 mL) to give ((2R,4S,5R)-5-amino-4-ethoxytetrahydro-2H-pyran-2-yl)((S)-1-(4-fluorophenyl)-3,4-dihydroisoquinolin-2(1H)-yl)methanone (COMPOUND 5088).

LCMS: 100%, RT=1.09 min., (M+H)⁺=399 (method P).

### Example 71

### ((2R,4S,5R)-5-amino-4-(difluoromethoxy)tetrahydro-2H-pyran-2-yl)((S)-1-(4-fluorophenyl)-3,4-dihydroisoquinolin-2(1H)-yl)methanone (COMPOUND 5089)

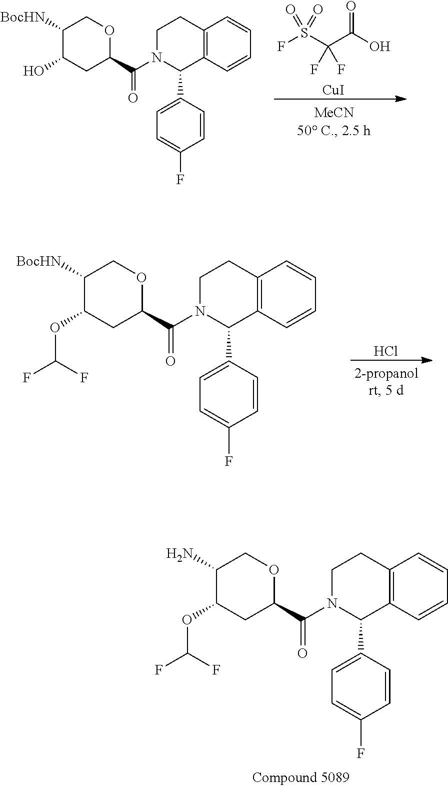

### Synthesis of tert-butyl ((3R,4S,6R)-4-(difluoromethoxy)-6-((S)-1-(4-fluorophenyl)-1,2,3,4-tetrahydroisoquinoline-2-carbonyl)tetrahydro-2H-pyran-3-yl)carbamate

Under an argon atmosphere, degassed acetonitrile (2 mL) was added to a vial containing tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (see Compound 5052, 50 mg, 0.106 mmol) and copper(I) iodide (5.1 mg, 0.027 mmol). The suspension was heated to 50° C. and a solution of 2,2-difluoro-2-(fluorosulfonyl)acetic acid (0.033 ml, 0.319 mmol) in degassed acetonitrile (0.2 mL) was added in portions over the course of 2 hours. After the addition, heating was continued for 30 minutes. The reaction mixture was diluted with dichloromethane (4 mL) and washed with saturated aqueous NaHCO₃ (3 mL). The layers were separated using a phase-separator and the organic filtrate was evaporated under reduced pressure. The residue was purified by flash column chromatography (silica, 0 to 50% ethyl acetate in heptane) to give tert-butyl ((3R,4S,6R)-4-(difluoromethoxy)-6-((S)-1-(4-fluorophenyl)-1,2,3,4-tetrahydroisoquinoline-2-carbonyl)tetrahydro-2H-pyran-3-yl)carbamate.

LCMS: 100%, RT=2.24 min., (M+H)⁺=521 (method A).

### Synthesis of ((2R,4S,5R)-5-amino-4-(difluoromethoxy)tetrahydro-2H-pyran-2-yl)((S)-1-(4-fluorophenyl)-3,4-dihydroisoquinolin-2(1H)-yl)methanone (COMPOUND 5089)

HCl (5-6 M in 2-propanol, 0.2 mL, 1.1 mmol) was added to a solution of tert-butyl ((3R,4S,6R)-4-(difluoromethoxy)-6-((S)-1-(4-fluorophenyl)-1,2,3,4-tetrahydroisoquinoline-2-carbonyl)tetrahydro-2H-pyran-3-yl)carbamate (19 mg, 0.036 mmol) in 2-propanol (2.0 mL). The mixture was stirred at room temperature for 5 days. The reaction mixture was diluted with dichloromethane (20 mL) and neutralised with saturated aqueous NaHCO₃. The layers were separated by a phase-separator and the organic filtrate was evaporated under reduced pressure. The residue was dissolved in methanol (1 mL) and brought onta an SCX-2 column (1 g) and eluted with methanol until neutral. Next, the column was eluted with ammonia in methanol (1.5 M). The basic fraction was concentrated to dryness under reduced pressure. The residue was lyophilised from a mixture of acetonitrile and water (1:2, 3 mL) to give ((2R,4S,5R)-5-amino-4-(difluoromethoxy)tetrahydro-2H-pyran-2-yl)((S)-1-(4-fluorophenyl)-3,4-dihydroisoquinolin-2(1H)-yl)methanone (COMPOUND 5089).

LCMS: 97%, RT=1.09 min., (M+H)⁺=421 (method P).

### Example 72

### ((2R,4R,5S)-4-amino-5-hydroxytetrahydro-2H-pyran-2-yl)((S)-1-(4-fluorophenyl)-3,4-dihydroisoquinolin-2(1H)-yl)methanone (COMPOUND 5092)

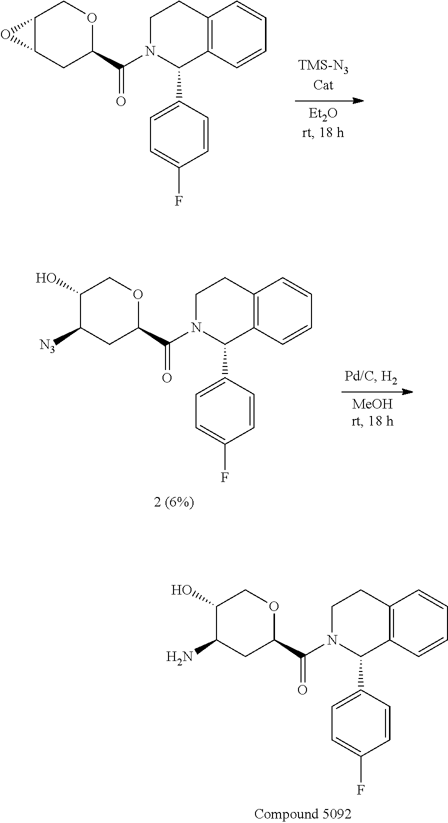 [SMILES: O=C([C@H]1C[C@@H]2O[C@@H]2CO1)N1CCc2ccccc2[C@@H]1c1ccc(F)cc1 ; [N-]=[N+]=N[C@@H]1C[C@H](C(=O)N2CCc3ccccc3[C@@H]2c2ccc(F)cc2)OC[C@H]1O ; N[C@@H]1C[C@H](C(=O)N2CCc3ccccc3[C@@H]2c2ccc(F)cc2)OC[C@H]1O]

### Synthesis of ((2R,4R,5S)-4-azido-5-hydroxytetrahydro-2H-pyran-2-yl)((S)-1-(4-fluorophenyl)-3,4-dihydroisoquinolin-2(1H)-yl)methanone

A solution of ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 230 mg, 0.651 mmol) in diethyl ether (dry, 4 mL) was added to a suspension of (1S,2S)-(+)-[1,2-cyclohexanediamino-N,N′-bis(3,5-di-t-butylsalicylidene)]chromium(III) chloride (Cat, 16.5 mg, 0.026 mmol) in diethyl ether (dry, 2 mL). After 15 minutes, trimethylsilyl azide (0.090 mL, 0.683 mmol) was added and the reaction mixture was stirred at room temperature for 18 hours. The mixture was concentrated under reduced pressure and the residue was purified by flash column chromatography (silica, 0 to 100% ethyl acetate in heptane) to give a mixture of the target compound and its regioisomer. This mixture was crystallized from methanol and the mother liquor was purified by preparative SFC (method BR) to give ((2R,4R,5S)-4-azido-5-hydroxytetrahydro-2H-pyran-2-yl)((S)-1-(4-fluorophenyl)-3,4-dihydroisoquinolin-2(1H)-yl)methanone.

LCMS SFC: RT=2.79 min., (M+H)⁺=396 (method V).

### Synthesis of ((2R,4R,5S)-4-amino-5-hydroxytetrahydro-2H-pyran-2-yl)((S)-1-(4-fluorophenyl)-3,4-dihydroisoquinolin-2(1H)-yl)methanone (COMPOUND 5092)

A solution of ((2R,4R,5S)-4-azido-5-hydroxytetrahydro-2H-pyran-2-yl)((S)-1-(4-fluorophenyl)-3,4-dihydroisoquinolin-2(1H)-yl)methanone (10 mg, 0.025 mmol) in methanol (extra dry, 2 mL) was hydrogenated in the presence of palladium (10 wt % on carbon, containing 50% water, 10.7 mg, 5 μmol) and atmospheric hydrogen pressure. After 18 hours, the reaction mixture was filtered and evaporated under reduced pressure. The residue was purified by preparative LCMS (method BU) to give

### ((2R,4R,5S)-4-amino-5-hydroxytetrahydro-2H-pyran-2-yl)((S)-1-(4-fluorophenyl)-3,4-dihydroisoquinolin-2(1H)-yl)methanone (COMPOUND 5092)

LCMS: 99%, RT=1.01 min., (M+H)⁺=371 (method P).

### Example 73

### ((2R,5R)-5-(bis(2-hydroxyethyl)amino)tetrahydro-2H-pyran-2-yl)((S)-1-(4-fluorophenyl)-3,4-dihydroisoquinolin-2(1H)-yl)methanone (COMPOUND 5093)

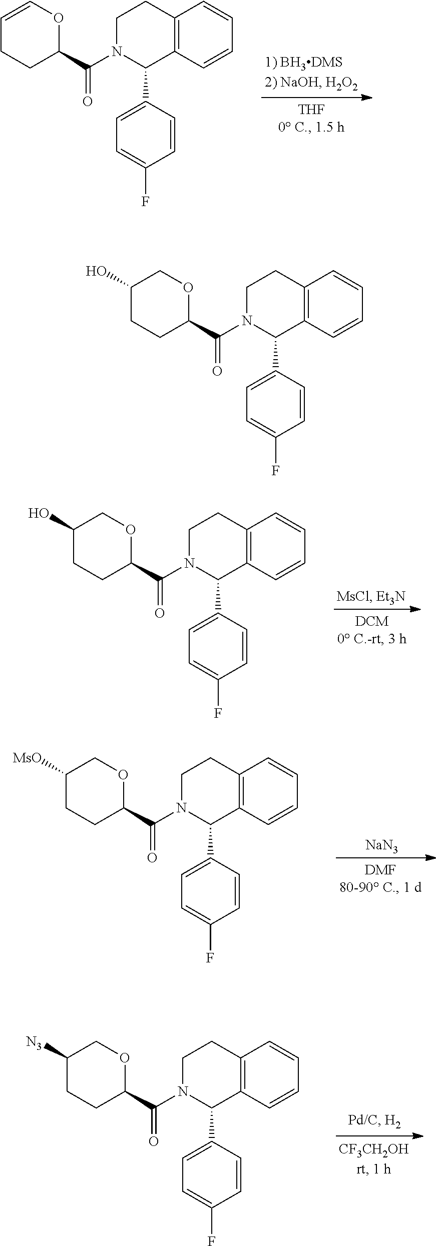

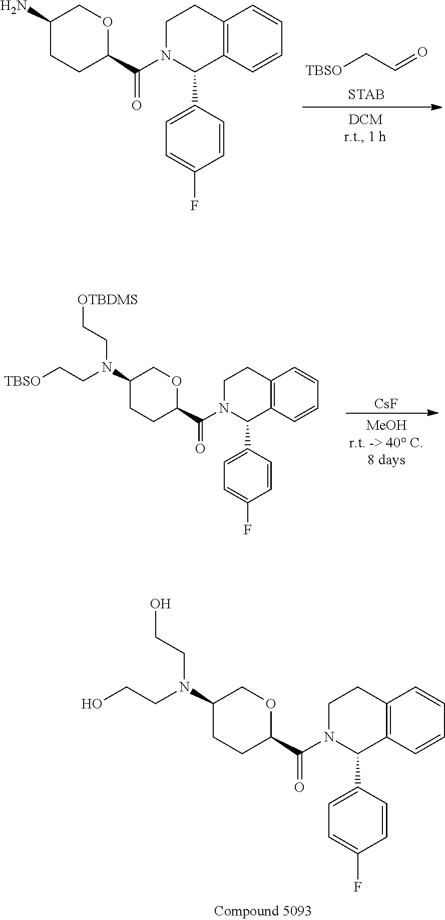

### Synthesis of ((S)-1-(4-fluorophenyl)-3,4-dihydroisoquinolin-2(1H)-yl)((2R,5S)-5-hydroxytetrahydro-2H-pyran-2-yl)methanone and ((S)-1-(4-fluorophenyl)-3,4-dihydroisoquinolin-2(1H)-yl)((2R,5R)-5-hydroxytetrahydro-2H-pyran-2-yl)methanone

At 0° C. under an argon atmosphere, borane-methyl sulfide complex (2M, 2.91 mL, 5.82 mmol) was added by syringe to a solution of ((R)-3,4-dihydro-2H-pyran-2-yl)((S)-1-(4-fluorophenyl)-3,4-dihydroisoquinolin-2(1H)-yl)methanone (see Compound 5213, 1.57 g, 4.65 mmol) in tetrahydrofuran (dry, 20 mL). After 1 hour, aqueous NaOH (1 M, 13.96 mL, 13.96 mmol) was slowly added followed by aqueous hydrogen peroxide (30% (w/v), 2.377 mL, 23.27 mmol). After 30 minutes, the mixture was diluted with water and extracted with ethyl acetate (twice). The combined organic layer was washed with brine, dried over Na₂SO₄, and concentrated under reduced pressure. The residue was purified by flash column chromatography (silica, 30 to 100% ethyl acetate in heptane) to give ((S)-1-(4-fluorophenyl)-3,4-dihydroisoquinolin-2(1H)-yl)((2R,5S)-5-hydroxytetrahydro-2H-pyran-2-yl)methanone, the first eluting isomer and ((S)-1-(4-fluorophenyl)-3,4-dihydroisoquinolin-2(1H)-yl)((2R,5R)-5-hydroxytetrahydro-2H-pyran-2-yl)methanone, the second eluting isomer.

First eluting isomer: SFC: 99%, RT=2.99 min., (M+H)⁺=356 (method AD).

Second eluting isomer: SFC: 60%, RT=3.51 min., (M+H)⁺=356 (method AD).

### Synthesis of (3S,6R)-6-((S)-1-(4-fluorophenyl)-1,2,3,4-tetrahydroisoquinoline-2-carbonyl)tetrahydro-2H-pyran-3-yl methanesulfonate

At 0° C. under an argon atmosphere, methanesulfonyl chloride (0.099 mL, 1.266 mmol) was added to a solution of ((S)-1-(4-fluorophenyl)-3,4-dihydroisoquinolin-2(1H)-yl)((2R,5S)-5-hydroxytetrahydro-2H-pyran-2-yl)methanone (300 mg, 0.844 mmol) and triethylamine (0.235 mL, 1.688 mmol) in dichloromethane. After 30 minutes, the mixture was allowed to warm up to room temperature and stirred for 1.5 hour. Additional triethylamine (0.082 mL, 0.591 mmol) and methanesulfonyl chloride (0.033 mL, 0.422 mmol) were added and the mixture was stirred for 1 more hour. Aqueous KHSO₄ (1 M) was added and the layers were separated over a phase separation filter. The organic filtrate was concentrated under reduced pressure to give (3S,6R)-6-((S)-1-(4-fluorophenyl)-1,2,3,4-tetrahydroisoquinoline-2-carbonyl)tetrahydro-2H-pyran-3-yl methanesulfonate.

LCMS: 95%, RT=2.07 min., (M+H)⁺=434 (method A).

### Synthesis of ((2R,5R)-5-azidotetrahydro-2H-pyran-2-yl)((S)-1-(4-fluorophenyl)-3,4-dihydroisoquinolin-2(1H)-yl)methanone

A solution of (3S,6R)-6-((S)-1-(4-fluorophenyl)-1,2,3,4-tetrahydroisoquinoline-2-carbonyl)tetrahydro-2H-pyran-3-yl methanesulfonate (366 mg, 0.844 mmol) and sodium azide (165 mg, 2.53 mmol) in N,N-dimethylformamide was heated at 80° C. and stirred overnight. Additional sodium azide (54.9 mg, 0.844 mmol) was added and heating was continued at 90° C. for 6 hours. After cooling to room temperature, the reaction mixture was diluted with water and extracted with ethyl acetate (twice). The combined organic layers were washed with brine (2×), dried over Na₂SO₄, and concentrated under reduced pressure. The residue was purified by flash column chromatography (silica, 5 to 50% ethyl acetate in heptane) to give ((2R,5R)-5-azidotetrahydro-2H-pyran-2-yl)((S)-1-(4-fluorophenyl)-3,4-dihydroisoquinolin-2(1H)-yl)methanone after co-evaporating from dichloromethane.

LCMS: 99%, RT=2.13 min., (M+H)⁺=381 (method A).

### Synthesis of ((2R,5R)-5-aminotetrahydro-2H-pyran-2-yl)((S)-1-(4-fluorophenyl)-3,4-dihydroisoquinolin-2(1H)-yl)methanone

A solution of ((2R,5R)-5-azidotetrahydro-2H-pyran-2-yl)((S)-1-(4-fluorophenyl)-3,4-dihydroisoquinolin-2(1H)-yl)methanone (222 mg, 0.584 mmol) in 2,2,2-trifluoroethanol (5 mL) was hydrogenated in the presence of palladium (10 wt % on carbon, containing 50% water, 150 mg, 0.070 mmol) at 45° C. and atmospheric hydrogen pressure. After 1 hour, the reaction mixture was filtered and the filtrate was concentrated under reduced pressure to give ((2R,5R)-5-aminotetrahydro-2H-pyran-2-yl)((S)-1-(4-fluorophenyl)-3,4-dihydroisoquinolin-2(1H)-yl)methanone that was used as such.

LCMS: 96%, RT=1.63 min., (M+H)⁺=355 (method A).

### Synthesis of ((2R,5R)-5-(bis(2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone

A solution of (tert-Butyldimethylsilyloxy)acetaldehyde (0.053 ml, 0.279 mmol) in dichloromethane (1 mL) was added to a solution of ((2R,5R)-5-aminotetrahydro-2H-pyran-2-yl)((S)-1-(4-fluorophenyl)-3,4-dihydroisoquinolin-2(1H)-yl)methanone (66 mg, 0.186 mmol) in dichloromethane (3 mL) and the mixture was stirred at room temperature for 20 minutes. Sodium triacetoxyborohydride (59 mg, 0.279 mmol) was added and stirring was continued for 1 hour. Saturated aqueous NaHCO₃ (5 mL) was added and the mixture was stirred for 15 minutes. The layers were separated over a phase separation filter. The organic filtrate was concentrated under reduced pressure and the residue was purified by flash column chromatography (silica, 5 to 50% ethyl acetate in heptane) to obtain ((2R,5R)-5-(bis(2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone.

LCMS: 98%, RT=3.76 min., (M+H)⁺=672 (method BV).

### Synthesis of ((2R,5R)-5-(bis(2-hydroxyethyl)amino)tetrahydro-2H-pyran-2-yl)((S)-1-(4-fluorophenyl)-3,4-dihydroisoquinolin-2(1H)-yl)methanone (COMPOUND 5093)

Cesium fluoride (60 mg, 0.393 mmol) was added to a solution of ((2R,5R)-5-(bis(2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone (44 mg, 0.066 mmol) in methanol (1 mL). The mixture was stirred at room temperature for 5 days followed by 3 days at 40° C. The mixture was brought onto an SCX-2 column (1 g) and eluted with methanol until neutral. Next, the column was eluted with ammonia in methanol (2 M). The basic fraction was concentrated to dryness under reduced pressure. The residue was lyophilised from a mixture of acetonitrile and water (1:1, 4 mL) to give ((2R,5R)-5-(bis(2-hydroxyethyl)amino)tetrahydro-2H-pyran-2-yl)((S)-1-(4-fluorophenyl)-3,4-dihydroisoquinolin-2(1H)-yl)methanone (COMPOUND 5093).

LCMS: 99%, RT=1.03 min., (M+H)⁺=443 (method P).

### Example 74

### ((S)-1-(4-fluorophenyl)-3,4-dihydroisoquinolin-2(1H)-yl)((2R,5R)-5-((2-methoxyethyl)amino)tetrahydro-2H-pyran-2-yl)methanone (COMPOUND 5212)

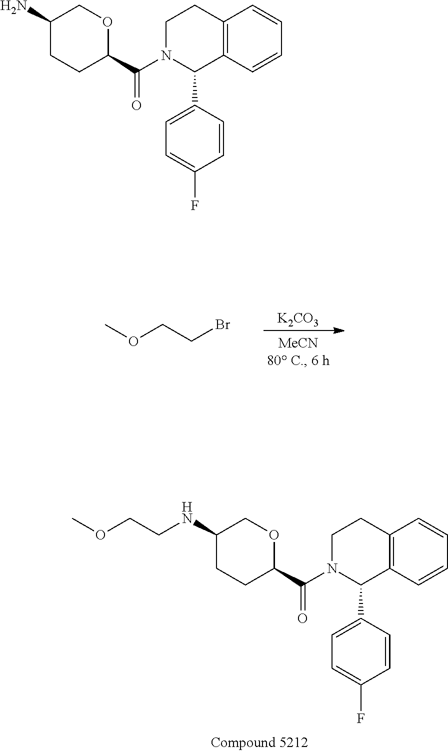

### Synthesis of ((S)-1-(4-fluorophenyl)-3,4-dihydroisoquinolin-2(1H)-yl)((2R,5R)-5-((2-methoxyethyl)amino)tetrahydro-2H-pyran-2-yl)methanone (COMPOUND 5212)

2-Bromoethyl methyl ether (0.021 mL, 0.226 mmol) was added to a suspension of ((2R,5R)-5-aminotetrahydro-2H-pyran-2-yl)((S)-1-(4-fluorophenyl)-3,4-dihydroisoquinolin-2(1H)-yl)methanone (see Compound 5093, 40 mg, 0.113 mmol) and potassium carbonate (47 mg, 0.339 mmol) in acetonitrile (2 mL). The mixture was heated at 80° C. for 6 hours. The mixture was purified by acidic preparative MPLC (Linear Gradient: t=0 min 5% A; t=1 min 20% A; t=16 min 60% A; t=17 min 100%; t=22 min 100% A; detection: 214/264 nm). The product fractions were concentrated under reduced pressure. The residue was dissolved in methanol (5 mL) and brought onto an SCX-2 column (1 g) and eluted with methanol until neutral. Next, the column was eluted with ammonia in methanol (2 M). The basic fraction was concentrated to dryness under reduced pressure. The residue was lyophilised from a mixture of acetonitrile and water (1:1, 4 mL) to give ((S)-1-(4-fluorophenyl)-3,4-dihydroisoquinolin-2(1H)-yl)((2R,5R)-5-((2-methoxyethyl)amino)tetrahydro-2H-pyran-2-yl)methanone (COMPOUND 5212).

LCMS: 98%, RT=1.07 min., (M+H)⁺=413 (method P).

### Example 75

### ((2S,4S)-4-amino-2-methyltetrahydro-2H-pyran-2-yl)((S)-1-(4-fluorophenyl)-3,4-dihydroisoquinolin-2(1H)-yl)methanone (COMPOUND 5238) and ((2S,4R)-4-amino-2-methyltetrahydro-2H-pyran-2-yl)((S)-1-(4-fluorophenyl)-3,4-dihydroisoquinolin-2(1H)-yl)methanone (COMPOUND 5237)

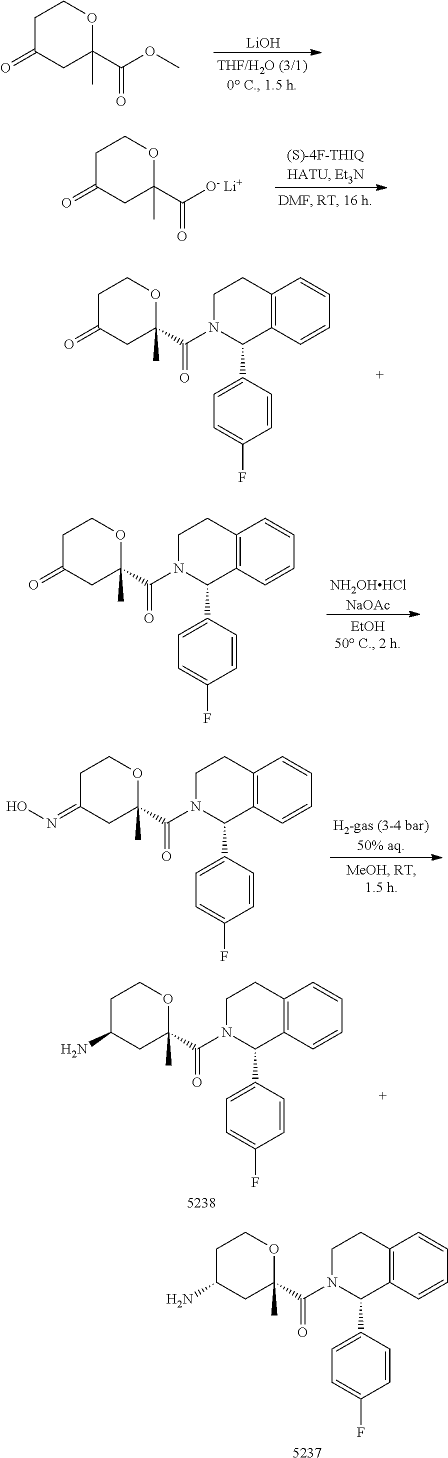

### Synthesis of lithium 2-methyl-4-oxotetrahydro-2H-pyran-2-carboxylate

At 0° C., aqueous LiOH (1.0 M, 4.98 mL, 4.98 mmol) was added dropwise to a solution of methyl 2-methyl-4-oxotetrahydro-2H-pyran-2-carboxylate (903 mg, 5.24 mmol) in tetrahydrofuran (30 mL) over the course of 1 hour. After 30 minutes, the reaction mixture was concentrated to dryness under reduced pressure to give lithium 2-methyl-4-oxotetrahydro-2H-pyran-2-carboxylate.

### Synthesis of (R)-2-((S)-1-(4-fluorophenyl)-1,2,3,4-tetrahydroisoquinoline-2-carbonyl)-2-methyltetra-hydro-4H-pyran-4-one and (S)-2-((S)-1-(4-fluorophenyl)-1,2,3,4-tetrahydroisoquinoline-2-carbonyl)-2-methyltetrahydro-4H-pyran-4-one

1-[Bis(dimethylamino)methylene]-1H-1,2,3-triazolo[4,5-b]pyridinium 3-oxid hexafluorophosphate (HATU, 3.11 g, 8.18 mmol), followed by triethylamine (1.36 mL, 9.82 mmol) were added to a solution of lithium 2-methyl-4-oxotetrahydro-2H-pyran-2-carboxylate (1.07 g, 6.55 mmol) in N,N-dimethylformamide (dry, 12.5 mL). After 10 minutes, (S)-1-(4-fluorophenyl)-1,2,3,4-tetrahydroisoquinoline (1.49 g, 6.55 mmol) was added and the reaction mixture was stirred for 16 hours. The reaction mixture was diluted with ethyl acetate (100 mL) and washed with saturated aqueous NaHCO₃ (100 mL). The aqueous layer was extracted with ethyl acetate (50 mL). The combined organics were washed with half saturated brine (3×25 mL) and brine (25 mL), dried over Na₂SO₄, and evaporated under reduced pressure. The residue was purified by flash column chromatography (silica, 0 to 50% ethyl acetate in heptane) and the diastereoisomers were separated by preparative SFC (method BR) to give (R)-2-((S)-1-(4-fluorophenyl)-1,2,3,4-tetrahydroisoquinoline-2-carbonyl)-2-methyltetrahydro-4H-pyran-4-one as the first eluting isomer on SFC and (S)-2-((S)-1-(4-fluorophenyl)-1,2,3,4-tetrahydroisoquinoline-2-carbonyl)-2-methyltetrahydro-4H-pyran-4-one as the second eluting isomer on SFC. Stereochemistry of the pyran ring was assigned arbitrarily.

First eluting isomer on SFC: LCMS: 99%, RT=2.08 min., (M+H)⁺=368 (method A, 40° C.). SFC: 99%, RT=1.93 min. (small plateau inbetween peak at 1.64 min.), (M+H)⁺=368 (method V).

Second eluting isomer on SFC: LCMS: 100%, RT=2.08 min., (M+H)⁺=368 (method A, 40° C.). SFC: 98%, RT=2.31 min., (M+H)⁺=368 (method V).

### Synthesis of ((S)-1-(4-fluorophenyl)-3,4-dihydroisoquinolin-2(1H)-yl)((S)-4-(hydroxyimino)-2-methyl-tetrahydro-2H-pyran-2-yl)methanone

(S)-2-((S)-1-(4-fluorophenyl)-1,2,3,4-tetrahydroisoquinoline-2-carbonyl)-2-methyltetrahydro-4H-pyran-4-one (200 mg, 0.544 mmol) was added to a suspension of hydroxylamine hydrochloride (76 mg, 1.09 mmol) and sodium acetate (89 mg, 1.09 mmol) in ethanol (absolute, 4.0 mL). The reaction vial was sealed and heated at 50° C. for 2 hours. After cooling to room temperature, the mixture was diluted with dichloromethane (15 mL) and washed with water (10 mL). The organic layer was passed through a phase-separator and evaporated under reduced pressure to give ((S)-1-(4-fluorophenyl)-3,4-dihydroisoquinolin-2(1H)-yl)((S)-4-(hydroxyimino)-2-methyl-tetrahydro-2H-pyran-2-yl). Stereochemistry of the pyran ring was assigned arbitrarily.

LCMS: 99%, RT=2.03 min., (M+H)⁺=383 (method A, 40° C.).

### Synthesis of ((2S,4S)-4-amino-2-methyltetrahydro-2H-pyran-2-yl)((S)-1-(4-fluorophenyl)-3,4-dihydroisoquinolin-2(1H)-yl)methanone (COMPOUND 5238) and ((2S,4R)-4-amino-2-methyltetrahydro-2H-pyran-2-yl)((S)-1-(4-fluorophenyl)-3,4-dihydroisoquinolin-2(1H)-yl)methanone (COMPOUND 5237)

To a stirred solution of ((S)-1-(4-fluorophenyl)-3,4-dihydroisoquinolin-2(1H)-yl)((S)-4-(hydroxyimino)-2-methyltetrahydro-2H-pyran-2-yl)methanone (148 mg, 0.387 mmol) in methanol (10 mL) in an autoclave was added 50% Raney®-Nickel slurry in water (0.50 mL). The reaction mixture was stirred under hydrogen atmosphere (3 bar) for 1.5 hours. The reaction mixture was diluted with methanol (10 mL) and filtered through a nylon filter. The filtrate was concentrated to dryness under reduced pressure. The residue was purified by flash column chromatography (silica, 0 to 5% (7M NH₃ in methanol) in dichloromethane) and the diastereoisomers were separated by preparative SFC (method BS) to give ((2S,4S)-4-amino-2-methyltetrahydro-2H-pyran-2-yl)((S)-1-(4-fluorophenyl)-3,4-dihydroisoquinolin-2(1H)-yl)methanone (COMPOUND 5238) as the first eluting isomer on SFC, and ((2S,4R)-4-amino-2-methyltetrahydro-2H-pyran-2-yl)((S)-1-(4-fluorophenyl)-3,4-dihydroisoquinolin-2(1H)-yl)methanone (COMPOUND 5237), the second eluting isomer on SFC, both after lyophilization from a mixture of acetonitrile and water (1:1, 4 mL). Stereochemistry of the pyran ring was assigned arbitrarily.

COMPOUND 5238: LCMS: 99%, RT=2.77 min., (M+H)⁺=369 (method AK, 40° C.). SFC: 99%, RT=2.39 min., (M+H)⁺=369 (method AS).

COMPOUND 5237: LCMS: 98%, RT=2.78 min., (M+H)⁺=369 (method AK, 40° C.). SFC: 95%, RT=2.59 min. (M+H)⁺=369 (method AS).

### Example 76

### ((2R,4S)-4-amino-2-methyltetrahydro-2H-pyran-2-yl)((S)-1-(4-fluorophenyl)-3,4-dihydroisoquinolin-2(1H)-yl)methanone (COMPOUND 5235) and ((2R,4R)-4-amino-2-methyltetrahydro-2H-pyran-2-yl)((S)-1-(4-fluorophenyl)-3,4-dihydroisoquinolin-2(1H)-yl) methanone (COMPOUND 5236)

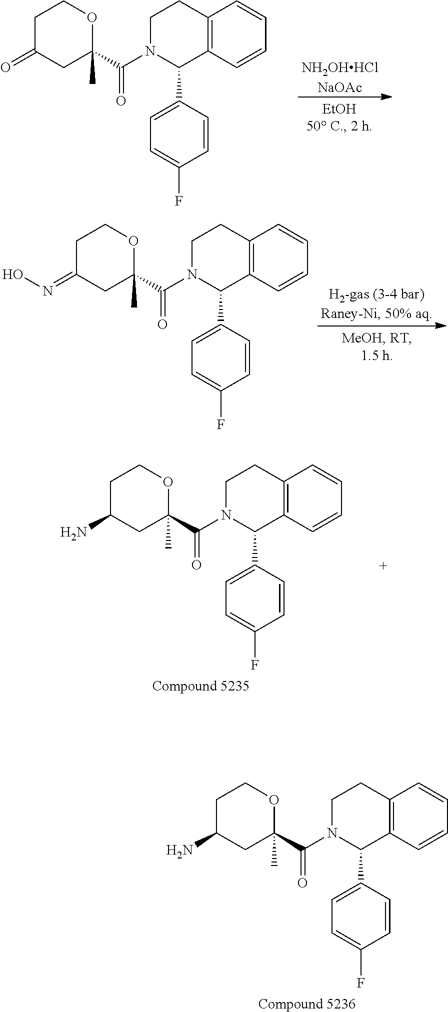

### Synthesis of ((S)-1-(4-fluorophenyl)-3,4-dihydroisoquinolin-2(1H)-yl)((R)-4-(hydroxyimino)-2-methyl-tetrahydro-2H-pyran-2-yl)methanone

Starting from (R)-2-((S)-1-(4-fluorophenyl)-1,2,3,4-tetrahydroisoquinoline-2-carbonyl)-2-methyltetrahydro-4H-pyran-4-one (see COMPOUND 5238, 200 mg, 0.544 mmol), ((S)-1-(4-fluorophenyl)-3,4-dihydroisoquinolin-2(1H)-yl)((R)-4-(hydroxyimino)-2-methyltetrahydro-2H-pyran-2-yl)methanone was prepared as described for ((S)-1-(4-fluorophenyl)-3,4-dihydroisoquinolin-2(1H)-yl) ((S)-4-(hydroxyimino)-2-methyltetrahydro-2H-pyran-2-yl)methanone (see COMPOUND 5238).

LCMS: 99%, RT=2.03 min., (M+H)⁺=383 (method A, 40° C.).

### Synthesis of ((2R,4S)-4-amino-2-methyltetrahydro-2H-pyran-2-yl)((S)-1-(4-fluorophenyl)-3,4-dihydroisoquinolin-2(1H)-yl)methanone (COMPOUND 5235) and ((2R,4R)-4-amino-2-methyltetrahydro-2H-pyran-2-yl)((S)-1-(4-fluorophenyl)-3,4-dihydroisoquinolin-2(1H)-yl)methanone (COMPOUND 5236)

Starting from ((S)-1-(4-fluorophenyl)-3,4-dihydroisoquinolin-2(1H)-yl)((R)-4-(hydroxyimino)-2-methyltetrahydro-2H-pyran-2-yl)methanone (134 mg, 0.350 mmol), ((2R,4S)-4-amino-2-methyltetrahydro-2H-pyran-2-yl)((S)-1-(4-fluorophenyl)-3,4-dihydroisoquinolin-2(1H)-yl)methanone (COMPOUND 5235) as the first eluting SFC (BEH column) isomer and ((2R,4R)-4-amino-2-methyltetrahydro-2H-pyran-2-yl)((S)-1-(4-fluorophenyl)-3,4-dihydroisoquinolin-2(1H)-yl)methanone (COMPOUND 5236) as the second eluting SFC (BEH column) isomer were prepared as described for ((2S,4S)-4-amino-2-methyltetrahydro-2H-pyran-2-yl)((S)-1-(4-fluorophenyl)-3,4-dihydroisoquinolin-2(1H)-yl)methanone (COMPOUND 5238) and purified by preparative chiral SFC (method BS and BT). Stereochemistry of the pyran ring was assigned arbitrarily.

COMPOUND 5235: white solid; LCMS: 99%, RT=2.82 min., (M+H)⁺=369 (method AK, 40° C.). SFC: 97%, RT=4.36 min., (M+H)⁺=369 (method F).

COMPOUND 5236: white solid. LCMS: 99%, RT=2.78 min., (M+H)⁺=369 (method AK, 40° C.). SFC: 99%, RT=4.07 min., (M+H)⁺=369 (method F).

### Example 77

### ((S)-1-(4-fluorophenyl)-3,4-dihydroisoquinolin-2(1H)-yl)((2R,5R)-5-((2-hydroxyethyl)amino)tetrahydro-2H-pyran-2-yl)methanone (COMPOUND 5211)

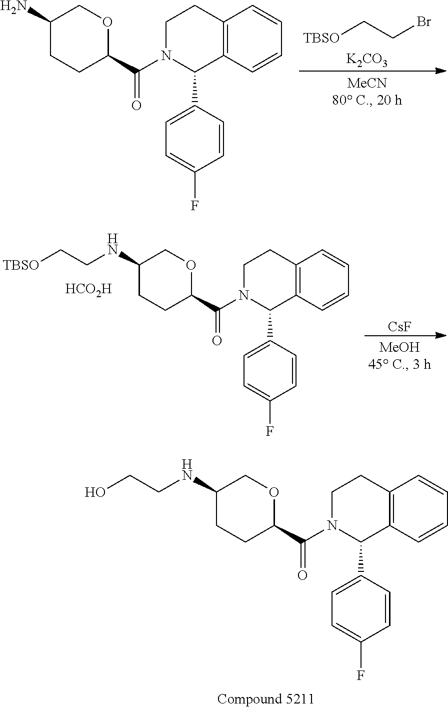

### Synthesis of ((2R,5R)-5-((2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone formate

(2-Bromoethoxy)-tert-butyldimethylsilane (0.061 mL, 0.282 mmol) was added to a suspension of ((2R,5R)-5-aminotetrahydro-2H-pyran-2-yl)((S)-1-(4-fluorophenyl)-3,4-dihydroisoquinolin-2(1H)-yl)methanone (see Compound 5093, 50 mg, 0.141 mmol) and potassium carbonate (59 mg, 0.423 mmol) in acetonitrile (2 mL). The mixture was heated at 80° C. for 20 hours. The mixture was purified by acidic preparative MPLC (Linear Gradient: t=0 min 5% A; t=1 min 20% A; t=16 min 60% A; t=17 min 100%; t=22 min 100% A; detection: 214/264 nm). The product fractions were concentrated under reduced pressure to give ((2R,5R)-5-((2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone formate (43 mg) as a syrup. LCMS: 81%, RT=1.94 min., (M+H)⁺=513 (method B).

### Synthesis of ((S)-1-(4-fluorophenyl)-3,4-dihydroisoquinolin-2(1H)-yl)((2R,5R)-5-((2-hydroxyethyl)amino)tetrahydro-2H-pyran-2-yl)methanone (COMPOUND 5211)

((2R,5R)-5-((2-((tert-butyldimethylsilyl)oxy)ethyl)amino)tetrahydro-2H-pyran-2-yl)((S)-1-(4-fluorophenyl)-3,4-dihydroisoquinolin-2(1H)-yl)methanone formate (43 mg, 0.077 mmol) was suspended in methanol (5 mL). Cesium fluoride (50 mg, 0.329 mmol) was added and the mixture was heated at 45° C. for 3 hours. The mixture was brought onto an SCX-2 column (1 g) and eluted with methanol until neutral. Next, the column was eluted with ammonia in methanol (2 M). The basic fraction was concentrated to dryness under reduced pressure. The residue was purified by basic preparative MPLC (Linear Gradient: t=0 min 5% A; t=1 min 20% A; t=16 min 60% A; t=17 min 100% A; t=22 min 100% A; detection: 214/264 nm). Product fractions were combined and concentrated under reduced pressure. The residue was lyophilised from a mixture of acetonitrile and water (1:1, 4 mL) to give ((S)-1-(4-fluorophenyl)-3,4-dihydroisoquinolin-2(1H)-yl)((2R,5R)-5-((2-hydroxyethyl)amino)tetrahydro-2H-pyran-2-yl)methanone (COMPOUND 5211).

LCMS: 99%, RT=1.03 min., (M+H)⁺=399 (method P).

### Example 78

### ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5213) and ((2S,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5214)

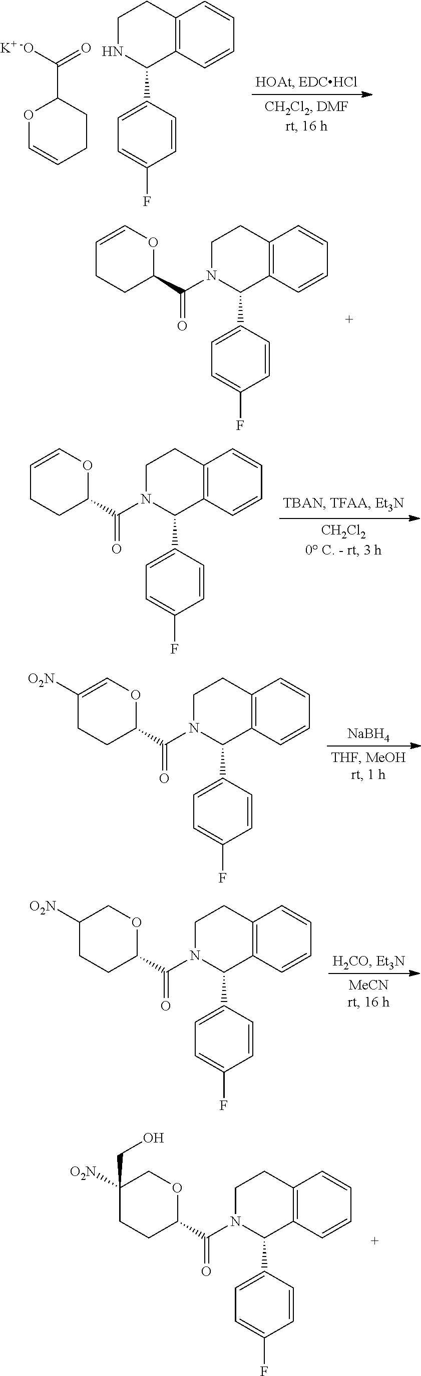

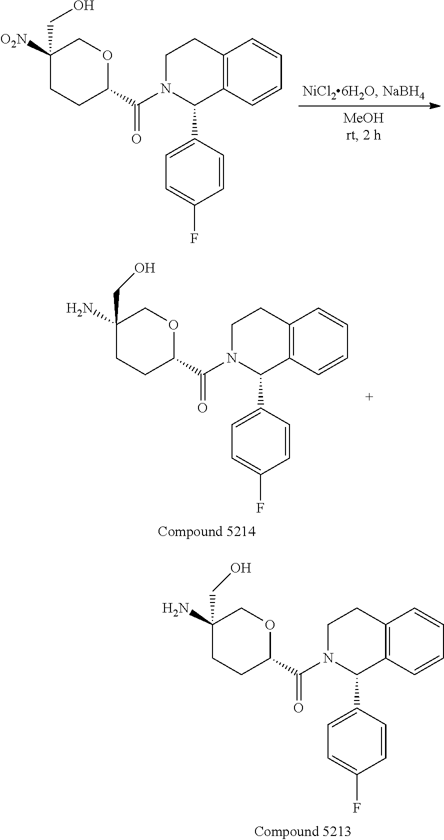 [SMILES: O=C([C@@H]1CC[C@@](CO)([N+](=O)[O-])CO1)N1CCc2ccccc2[C@@H]1c1ccc(F)cc1 ; N[C@@]1(CO)CC[C@@H](C(=O)N2CCc3ccccc3[C@@H]2c2ccc(F)cc2)OC1 ; N[C@]1(CO)CC[C@@H](C(=O)N2CCc3ccccc3[C@@H]2c2ccc(F)cc2)OC1]

### Synthesis of ((S)-3,4-dihydro-2H-pyran-2-yl)((S)-1-(4-fluorophenyl)-3,4-dihydroisoquinolin-2(1H)-yl)methanone and ((R)-3,4-dihydro-2H-pyran-2-yl)((S)-1-(4-fluorophenyl)-3,4-dihydroisoquinolin-2(1H)-yl)methanone

N-(3-dimethylaminopropyl)-N′-ethylcarbodiimide hydrochloride (1.816 g, 9.48 mmol) and 1-hydroxy-7-azabenzotriazole (0.123 g, 0.902 mmol) were added to a suspension of (S)-1-(4-fluorophenyl)-1,2,3,4-tetrahydroisoquinoline (2.154 g, 9.48 mmol) and potassium 3,4-dihydro-2H-pyran-2-carboxylate (1.5 g, 9.02 mmol) in a mixture of dichloromethane (30 mL) and N,N-dimethylformamide (dry, 7.5 mL). After 20 hours, the reaction mixture was partially concentrated under reduced pressure. The residue was diluted with aqueous HCl (0.5 M, 50 mL) and extracted with ethyl acetate (250 mL). The organic layer was washed with water (50 mL) and saturated aqueous NaHCO₃ (50 mL), dried over Na₂SO₄, and concentrated to dryness under reduced pressure. The residue was purified by flash column chromatography (silica, 0 to 15% ethyl acetate in heptane) to give ((R)-3,4-dihydro-2H-pyran-2-yl)((S)-1-(4-fluorophenyl)-3,4-dihydroisoquinolin-2(1H)-yl)methanone as the first eluting isomer and ((S)-3,4-dihydro-2H-pyran-2-yl)((S)-1-(4-fluorophenyl)-3,4-dihydroisoquinolin-2(1H)-yl)methanone as the second eluting isomer.

First eluting isomer: LCMS: 99%, RT=2.14 min., (M+H)⁺=338 (method A). SFC: 99%, RT=2.79 min., (M+H)⁺=338 (method F).

Second eluting isomer: LCMS: 99%, RT=2.14 min., (M+H)⁺=338 (method A). SFC: 99%, RT=2.61 min., (M+H)⁺=338 (method F)

### Synthesis of ((S)-1-(4-fluorophenyl)-3,4-dihydroisoquinolin-2(1H)-yl)((S)-5-nitro-3,4-dihydro-2H-pyran-2-yl)methanone

Under an argon atmosphere, trifluoroacetic anhydride (0.142 mL, 1.024 mmol) was added to a solution of ((S)-3,4-dihydro-2H-pyran-2-yl)((S)-1-(4-fluorophenyl)-3,4-dihydroisoquinolin-2(1H)-yl)methanone (314 mg, 0.931 mmol) and tetrabutylammonium nitrate (312 mg, 1.024 mmol) in dichloromethane (5 mL) at 0° C. The mixture was allowed to reach room temperature and stirred for 3 hours. The reaction mixture was cooled to 0° C. and triethylamine (0.129 mL, 0.931 mmol) was added dropwise. After 15 minutes, the reaction mixture was diluted with ice/water (10 mL). The layers were separated using a phase separator and the organic filtrate was evaporated under reduced pressure. The residue was purified by flash column chromatography (silica, 0 to 100% ethyl acetate in heptane) to give ((S)-1-(4-fluorophenyl)-3,4-dihydroisoquinolin-2(1H)-yl)((S)-5-nitro-3,4-dihydro-2H-pyran-2-yl)methanone.

LCMS: 84%, RT=2.10 min., (M+H)⁺=383 (method A).

### Synthesis of ((S)-1-(4-fluorophenyl)-3,4-dihydroisoquinolin-2(1H)-yl)((2S,5R)-5-nitrotetrahydro-2H-pyran-2-yl)methanone and ((S)-1-(4-fluorophenyl)-3,4-dihydroisoquinolin-2(1H)-yl)((2S,5S)-5-nitrotetrahydro-2H-pyran-2-yl)methanone

Sodium borohydride (18.6 mg, 0.490 mmol) was added to a solution of ((S)-1-(4-fluorophenyl)-3,4-dihydroisoquinolin-2(1H)-yl)((S)-5-nitro-3,4-dihydro-2H-pyran-2-yl)methanone (150 mg, 0.392 mmol) in a mixture of tetrahydrofuran (dry, 10 mL) and methanol (dry, 1 mL). After 1 hour, water (1 mL) was added and the mixture was concentrated to dryness under reduced pressure. The residue was dissolved in ethyl acetate (5 mL) and washed with brine (3 mL). The combined organics were dried over Na₂SO₄ and evaporated under reduced pressure. The residue was purified by flash column chromatography (silica, 0 to 100% ethyl acetate in heptane) to give ((S)-1-(4-fluorophenyl)-3,4-dihydroisoquinolin-2(1H)-yl)((2S,5R)-5-nitrotetrahydro-2H-pyran-2-yl)methanone as the first eluting isomer and ((S)-1-(4-fluorophenyl)-3,4-dihydroisoquinolin-2(1H)-yl)((2S,5S)-5-nitrotetrahydro-2H-pyran-2-yl)methanone as the second eluting isomer. The stereochemistry 5-nitro-pyran stereocenter was arbitrarily assigned.

First eluting isomer: LCMS: 99%, RT=2.11 min., (M+H)⁺=385 (method A).

Second eluting isomer: LCMS: 99%, RT=2.07 min., (M+H)⁺=385 (method A).

### Synthesis of ((S)-1-(4-fluorophenyl)-3,4-dihydroisoquinolin-2(1H)-yl)((2S,5S)-5-(hydroxymethyl)-5-nitrotetrahydro-2H-pyran-2-yl)methanone and ((S)-1-(4-fluorophenyl)-3,4-dihydroisoquinolin-2(1H)-yl)((2S,5R)-5-(hydroxymethyl)-5-nitrotetrahydro-2H-pyran-2-yl)methanone

Formaldehyde (37% wt solution in H₂O, 0.050 mL, 0.663 mmol) and triethylamine (0.031 mL, 0.221 mmol) were added to a solution of a mixture of ((S)-1-(4-fluorophenyl)-3,4-dihydroisoquinolin-2(1H)-yl)((2S,5R)-5-nitrotetrahydro-2H-pyran-2-yl)methanone (37 mg, 0.096 mmol) and ((S)-1-(4-fluorophenyl)-3,4-dihydroisoquinolin-2(1H)-yl)((2S,5S)-5-nitrotetrahydro-2H-pyran-2-yl)methanone (48 mg, 0.125 mmol) in acetonitrile (4 ml). After 16 hours, the reaction mixture was concentrated under reduced pressure and the residue was co-evaporated from methanol (2 mL). The residue was purified by flash column chromatography (silica, 0 to 100% ethyl acetate in heptane) to give ((S)-1-(4-fluorophenyl)-3,4-dihydroisoquinolin-2(1H)-yl)((2S,5R)-5-(hydroxymethyl)-5-nitrotetrahydro-2H-pyran-2-yl)methanone as the first eluting isomer, and ((S)-1-(4-fluorophenyl)-3,4-dihydroisoquinolin-2(1H)-yl)((2S,5S)-5-(hydroxymethyl)-5-nitrotetrahydro-2H-pyran-2-yl)methanone as the second eluting isomer. The stereochemistry 5-nitro-pyran stereocenter was arbitrarily assigned.

First eluting isomer: LCMS: 99%, RT=2.03 min., (M+H)⁺=415 (method A).

Second eluting isomer (silica): LCMS: 99%, RT=1.99 min., (M+H)⁺=415 (method A).

### Synthesis of ((2S,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5214)

Nickel(II) chloride hexahydrate (19.9 mg, 0.084 mmol) was added to a solution of ((S)-1-(4-fluorophenyl)-3,4-dihydroisoquinolin-2(1H)-yl)((2S,5R)-5-(hydroxymethyl)-5-nitrotetrahydro-2H-pyran-2-yl)methanone (33 mg, 0.080 mmol) in methanol (6 mL). The reaction mixture was cooled to 0° C. and sodium borohydride (26.3 mg, 0.695 mmol) was added in two portions. Then the mixture was warmed to room temperature. After 2 hours water (1.5 mL) was added and stirring was continued for 30 minutes. The mixture was diluted with methanol (5 mL) and filtered over a nylon 0.45 μm filter. The filter was rinsed with a mixture of methanol and dichloromethane (1:1, 2×5 mL). The combined filtrate was concentrated to dryness under reduced pressure. The residue was stirred in a mixture of dichloromethane (5 mL) and water (2 mL) for 5 minutes. The layers were separated using a phase-separator and the organic filtrate was evaporated under reduced pressure. The residue was purified by basic preparative MPLC (Linear Gradient: t=0 min 5% A; t=1 min 5% A; t=16 min 60% A; t=17 min 100% A; t=22 min 100% A; detection: 251/210 nm) and the product containing fractions were lyophilized from a mixture of acetonitrile and water (1:1, 6 mL) to give ((2S,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5214). The stereochemistry 5-amino-pyran stereocenter was arbitrarily assigned.

LCMS: 99%, RT=1.01 min., (M+H)⁺=385 (method P). SFC: 99%, RT=4.70 min., (M+H)⁺=385 (method F).

### Synthesis of ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5213)

Starting from ((S)-1-(4-fluorophenyl)-3,4-dihydroisoquinolin-2(1H)-yl)((2S,5S)-5-(hydroxymethyl)-5-nitrotetrahydro-2H-pyran-2-yl)methanone (36 mg, 0.087 mmol), ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5213) was prepared as described for ((2S,5R)-5-amino-5-(hydroxymethyl)tetrahydro-2H-pyran-2-yl)((S)-1-(4-fluorophenyl)-3,4-dihydroisoquinolin-2(1H)-yl)methanone (see COMPOUND 5214). The stereochemistry 5-amino-pyran stereocenter was arbitrarily assigned.

LCMS: 99%, RT=1.02 min., (M+H)⁺=385 (method P). SFC: 99%, RT=4.35 min., (M+H)⁺=385 (method F).

### Example 79

### ((6S,7S)-6-fluoro-1,4-oxazepan-7-yl)((S)-1-(4-fluorophenyl)-3,4-dihydroisoquinolin-2(1H)-yl)methanone (COMPOUND 5418) and ((2R,3R)-3-(fluoromethyl)morpholin-2-yl)((S)-1-(4-fluorophenyl)-3,4-dihydroisoquinolin-2(1H)-yl)methanone (COMPOUND 5420)

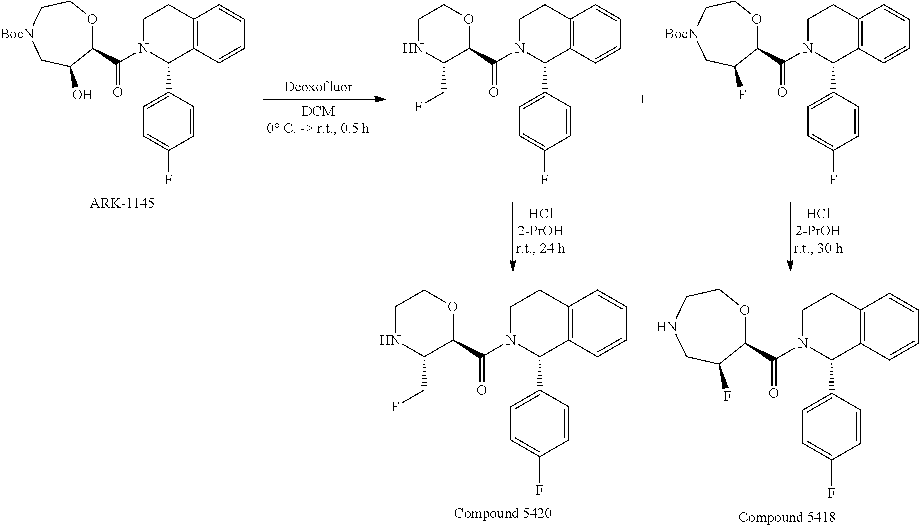 [SMILES: CC(C)(C)OC(=O)N1CCO[C@@H](C(=O)N2CCc3ccccc3[C@@H]2c2ccc(F)cc2)[C@@H](O)C1 ; O=C([C@@H]1OCCN[C@H]1CF)N1CCc2ccccc2[C@@H]1c1ccc(F)cc1 ; CC(C)(C)OC(=O)N1CCO[C@@H](C(=O)N2CCc3ccccc3[C@@H]2c2ccc(F)cc2)[C@@H](F)C1 ; O=C([C@@H]1OCCN[C@H]1CF)N1CCc2ccccc2[C@@H]1c1ccc(F)cc1 ; O=C([C@@H]1OCCNC[C@@H]1F)N1CCc2ccccc2[C@@H]1c1ccc(F)cc1]

### Synthesis of tert-butyl (6S,7S)-6-fluoro-7-((S)-1-(4-fluorophenyl)-1,2,3,4-tetrahydroisoquinoline-2-carbonyl)-1,4-oxazepane-4-carboxylate and tert-butyl ((2R,3R)-3-(fluoromethyl)morpholin-2-yl)((S)-1-(4-fluorophenyl)-3,4-dihydroisoquinolin-2(1H)-yl)methanone

Deoxofluor (0.102 mL, 0.276 mmol) was added to a solution of tert-butyl (6S,7R)-7-((S)-1-(4-fluorophenyl)-1,2,3,4-tetrahydroisoquinoline-2-carbonyl)-6-hydroxy-1,4-oxazepane-4-carboxylate (see COMPOUND 5243, 118 mg, 0.251 mmol) in dichloromethane (2 mL) at 0° C. The reaction mixture was allowed to warm up and stirred at room temperature for 30 minutes. The reaction mixture was diluted with dichloromethane (10 mL) and saturated aqueous NaHCO₃ (10 mL). The layers were separated using a phase-separator and the organic filtrate was evaporated under reduced pressure. The residue was purified by flash column chromatography (silica, 0 to 30% ethyl acetate in heptane) to give tert-butyl ((2R,3R)-3-(fluoromethyl)morpholin-2-yl)((S)-1-(4-fluorophenyl)-3,4-dihydroisoquinolin-2(1H)-yl)methanone as the first eluting product on silica and tert-butyl (6S,7R)-6-fluoro-7-((S)-1-(4-fluorophenyl)-1,2,3,4-tetrahydroisoquinoline-2-carbonyl)-1,4-oxazepane-4-carboxylate as the second eluting product on silica. Morpholine and oxazepane stereochemistry are arbitrarily assigned.

First eluting product: LCMS: 100%, RT=2.23 min., (M+Na)⁺=495 (method A).

Second eluting product: LCMS: 100%, RT=2.21 min., (M+Na)⁺=495 (method A).

### Synthesis of ((2R,3R)-3-(fluoromethyl)morpholin-2-yl)((S)-1-(4-fluorophenyl)-3,4-dihydroisoquinolin-2(1H)-yl)methanone (COMPOUND 5420)

HCl (5-6 M in 2-propanol, 1.0 mL, 6.00 mmol) was added to a solution of tert-butyl ((2R,3R)-3-(fluoromethyl)morpholin-2-yl)((S)-1-(4-fluorophenyl)-3,4-dihydroisoquinolin-2(1H)-yl)methanone (28 mg, 0.059 mmol) in 2-propanol (5 mL). The mixture was stirred at room temperature for 24 hours. The mixture was diluted with dichloromethane (5 mL) and saturated aqueous NaHCO₃ (5 mL). The layers were separated over a phase separator and the organic filtrate was concentrated under reduced pressure. The residue was lyophilized from a mixture of acetonitrile and water (1:1, 4 mL) to give ((6R,7S)-6-fluoro-1,4-oxazepan-7-yl)((S)-1-(4-fluorophenyl)-3,4-dihydroisoquinolin-2(1H)-yl)methanone (COMPOUND 5420). Morpholine stereochemistry is arbitrarily assigned.

LCMS: 97%, RT=1.07 min., (M+H)⁺=373 (method P).

### Synthesis of ((6S,7S)-6-fluoro-1,4-oxazepan-7-yl)((S)-1-(4-fluorophenyl)-3,4-dihydroisoquinolin-2(1H)-yl)methanone (COMPOUND 5418)

HCl (5-6 M in 2-propanol, 1.0 mL, 6.00 mmol) was added to a solution of tert-butyl (6S,7S)-6-fluoro-7-((S)-1-(4-fluorophenyl)-1,2,3,4-tetrahydroisoquinoline-2-carbonyl)-1,4-oxazepane-4-carboxylate (58 mg, 0.123 mmol) in 2-propanol (5 mL). The mixture was stirred at room temperature for 24 hours. HCl (5-6 M in 2-propanol, 1.0 mL, 6.00 mmol) was added and the mixture was stirred at room temperature for 6 hours. The mixture was diluted with dichloromethane (5 mL) and saturated aqueous NaHCO₃ (5 mL). The layers were separated over a phase separator and the organic filtrate was concentrated under reduced pressure. The residue was lyophilised from a mixture of acetonitrile and water (1:1, 4 mL) to give ((6S,7S)-6-fluoro-1,4-oxazepan-7-yl)((S)-1-(4-fluorophenyl)-3,4-dihydroisoquinolin-2(1H)-yl)methanone (COMPOUND 5418). Oxazepane stereochemistry is arbitrarily assigned.

LCMS: 97%, RT=1.06 min., (M+H)⁺=373 (method P).

### Example 80

### ((S)-1-(4-fluorophenyl)-3,4-dihydroisoquinolin-2(1H)-yl)((6R,7S)-6-hydroxy-1,4-oxazepan-7-yl)methanone (COMPOUND 5246) and ((S)-1-(4-fluorophenyl)-3,4-dihydroisoquinolin-2(1H)-yl)((6S,7R)-6-hydroxy-1,4-oxazepan-7-yl)methanone (COMPOUND 5243)

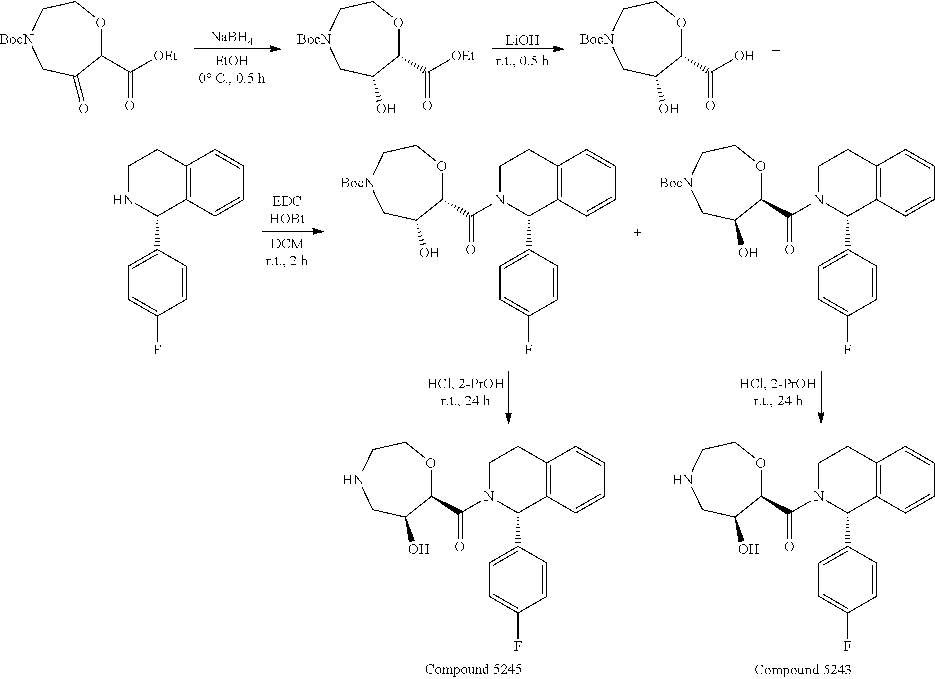

### Synthesis of 4-(tert-butyl) 7-ethyl cis-6-hydroxy-1,4-oxazepane-4,7-dicarboxylate

At 0° C., sodium borohydride (263 mg, 6.96 mmol) was added to a solution of 4-(tert-butyl) 7-ethyl 6-oxo-1,4-oxazepane-4,7-dicarboxylate (2.00 g, 6.96 mmol) in ethanol (100 mL, required heating). After 30 minutes at 0° C., the mixture was diluted with saturated aqueous NH₄Cl (20 mL), brine (50 mL) and ethyl acetate (300 mL) and the layers were separated. The organic layer was washed with brine (50 mL), dried over Na₂SO₄, and evaporated under reduced pressure. The residue was purified by flash column chromatography (silica, 20 to 100% ethyl acetate in heptane) to give 4-(tert-butyl) 7-ethyl cis-6-hydroxy-1,4-oxazepane-4,7-dicarboxylate (543 mg) as the first eluting diastereoisomer, and 4-(tert-butyl) 7-ethyl trans-6-hydroxy-1,4-oxazepane-4,7-dicarboxylate as the second eluting diastereoisomer. Oxazepane relative stereochemistry is arbitrarily assigned and a racemic mixture.

First eluting diastereoisomer: LCMS: 99%, RT=1.70 min., (2M+Na)⁺=601 (method A).

Second eluting diastereoisomer: LCMS: 99%, RT=1.65 min., (2M+Na)⁺=601 (method A).

### Synthesis of cis-4-(tert-butoxycarbonyl)-6-hydroxy-1,4-oxazepane-7-carboxylic acid

A solution of lithium hydroxide monohydrate (152 mg, 3.63 mmol) in water (7 mL) was added to a solution of 4-(tert-butyl) 7-ethyl cis-6-hydroxy-1,4-oxazepane-4,7-dicarboxylate (700 mg, 2.419 mmol) in ethanol (21 mL). After 30 minutes, the reaction mixture was diluted with a mixture of chloroform and 2-propanol (2:1, 120 mL) and acidified with a mixture of aqueous HCl (1 M) and water (1:1, 30 mL). The organic layer was collected and the aqueous layer was extracted with a mixture of chloroform and 2-propanol (2:1, 2×60 mL). The combined organic phase was dried over Na₂SO₄ and evaporated under reduced pressure to give cis-4-(tert-butoxycarbonyl)-6-hydroxy-1,4-oxazepane-7-carboxylic acid. Oxazepane relative stereochemistry is arbitrarily assigned and a racemic mixture.

LCMS: 100%, RT=1.51 min., (M-tBu+H)⁺=206 (method A).

### Synthesis of tert-butyl (6R,7S)-7-((S)-1-(4-fluorophenyl)-1,2,3,4-tetrahydroisoquinoline-2-carbonyl)-6-hydroxy-1,4-oxazepane-4-carboxylate and tert-butyl (6S,7R)-7-((S)-1-(4-fluorophenyl)-1,2,3,4-tetrahydroisoquinoline-2-carbonyl)-6-hydroxy-1,4-oxazepane-4-carboxylate

1-Ethyl-3-(3-dimethylaminopropyl)carbodiimide hydrochloride (462 mg, 2.411 mmol) was added to a solution of cis-4-(tert-Butoxycarbonyl)-6-hydroxy-1,4-oxazepane-7-carboxylic acid (630 mg, 2.41 mmol) and (S)-1-(4-fluorophenyl)-1,2,3,4-tetrahydroisoquinoline (603 mg, 2.65 mmol) in dichloromethane (7 mL). The reaction mixture was stirred for 2 minutes, hydroxybenzotriazole hydrate (73.9 mg, 0.482 mmol) was added and the mixture was stirred at room temperature for 2 hours. The reaction mixture was diluted with dichloromethane (30 mL) and washed with saturated aqueous NaHCO₃ (20 mL), aqueous HCl (1 M, 20 mL), and brine (15 mL), dried over Na₂SO₄ and concentrated under reduced pressure. The residue was purified by flash column chromatography (silica, 10 to 30% ethyl acetate in heptane) twice to give tert-butyl (6R,7S)-7-((S)-1-(4-fluorophenyl)-1,2,3,4-tetrahydroisoquinoline-2-carbonyl)-6-hydroxy-1,4-oxazepane-4-carboxylate as the first eluting diastereoisomer and tert-butyl (6S,7R)-7-((S)-1-(4-fluorophenyl)-1,2,3,4-tetrahydroisoquinoline-2-carbonyl)-6-hydroxy-1,4-oxazepane-4-carboxylate as the second eluting diastereoisomer. Oxazepane stereochemistry is arbitrarily assigned.

First eluting diastereoisomer: LCMS: 100%, RT=2.11 min., (M-tBu+H)⁺=415 (method A).

Second eluting diastereoisomer: LCMS: 100%, RT=2.10 min., (M-tBu+H)⁺=415 (method A).

### Synthesis of ((S)-1-(4-fluorophenyl)-3,4-dihydroisoquinolin-2(1H)-yl)((6R,7S)-6-hydroxy-1,4-oxazepan-7-yl)methanone (COMPOUND 5246)

HCl (5-6 M in 2-propanol, 1.0 mL, 6.00 mmol) was added to a solution of tert-butyl (6R,7S)-7-((S)-1-(4-fluorophenyl)-1,2,3,4-tetrahydroisoquinoline-2-carbonyl)-6-hydroxy-1,4-oxazepane-4-carboxylate (50 mg, 0.106 mmol) in 2-propanol (2 mL). After 24 hours, the mixture was diluted with dichloromethane (10 mL) and saturated aqueous NaHCO₃ (15 mL). The layers were separated over a phase separator and the organic filtrate was concentrated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure and the residue was lyophilized from a mixture of acetonitrile and water (1:2, 3 mL) to give ((S)-1-(4-fluorophenyl)-3,4-dihydroisoquinolin-2(1H)-yl)((6R,7S)-6-hydroxy-1,4-oxazepan-7-yl)methanone (COMPOUND 5246). Oxazepane stereochemistry is arbitrarily assigned.

LCMS: 99%, RT=1.02 min., (M+H)⁺=371 (method P).

### Synthesis of ((S)-1-(4-fluorophenyl)-3,4-dihydroisoquinolin-2(1H)-yl)((6S,7R)-6-hydroxy-1,4-oxazepan-7-yl)methanone (COMPOUND 5243)

HCl (5-6 M in 2-propanol, 1.0 mL, 6.00 mmol) was added to a solution of tert-butyl (6S,7R)-7-((S)-1-(4-fluorophenyl)-1,2,3,4-tetrahydroisoquinoline-2-carbonyl)-6-hydroxy-1,4-oxazepane-4-carboxylate (36 mg, 0.077 mmol) in 2-propanol (1 mL). After 24 hours, the mixture was diluted with dichloromethane (10 mL) and saturated aqueous NaHCO₃ (10 mL). The layers were separated over a phase separator and the organic filtrate was concentrated under reduced pressure. The residue was dissolved in methanol (1 mL) and brought onto an SCX-2 column (1 g) and eluted with methanol until neutral. Next, the column was eluted with ammonia in methanol (1.5 M). The basic fraction was concentrated to dryness under reduced pressure and the residue was lyophilized from a mixture of acetonitrile and water (1:2, 3 mL) to give ((S)-1-(4-fluorophenyl)-3,4-dihydroisoquinolin-2(1H)-yl)((6S,7R)-6-hydroxy-1,4-oxazepan-7-yl)methanone (COMPOUND 5243). Oxazepane stereochemistry is arbitrarily assigned.

LCMS: 99%, RT=1.03 min., (M+H)⁺=371 (method P).

### Example 81

### ((2R,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5215) and ((2R,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5216)

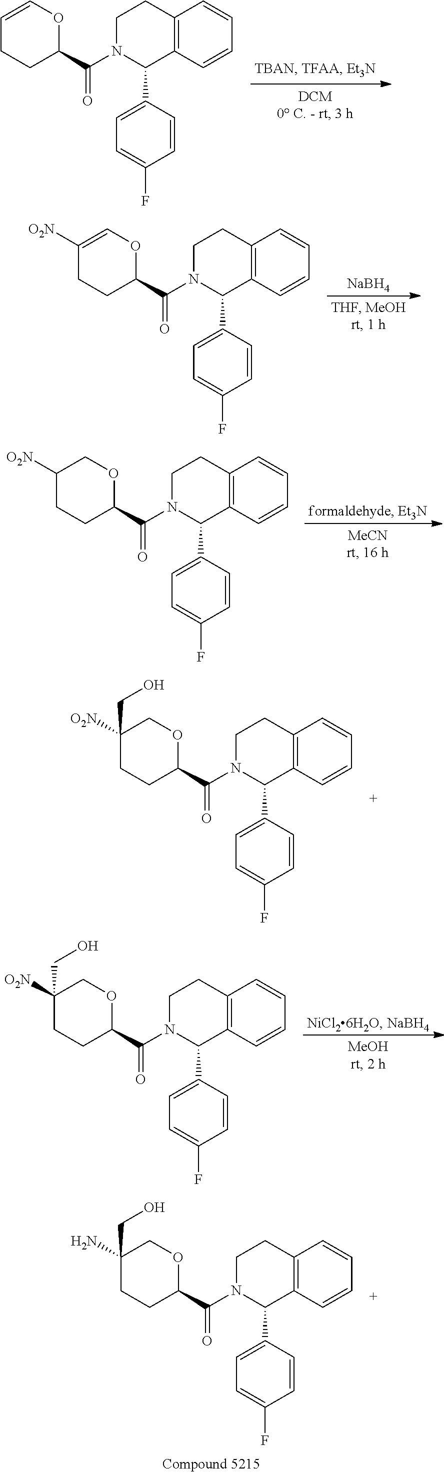

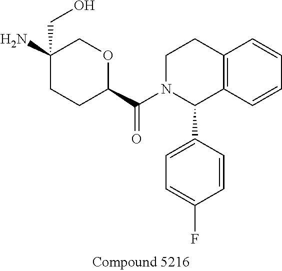 [SMILES: N[C@]1(CO)CC[C@H](C(=O)N2CCc3ccccc3[C@@H]2c2ccc(F)cc2)OC1]

### Synthesis of ((S)-1-(4-fluorophenyl)-3,4-dihydroisoquinolin-2(1H)-yl)((R)-5-nitro-3,4-dihydro-2H-pyran-2-yl)methanone

Starting from ((R)-3,4-dihydro-2H-pyran-2-yl)((S)-1-(4-fluorophenyl)-3,4-dihydroisoquinolin-2(1H)-yl)methanone (see COMPOUND 5213, 1.1 g, 3.26 mmol), ((S)-1-(4-fluorophenyl)-3,4-dihydroisoquinolin-2(1H)-yl)((R)-5-nitro-3,4-dihydro-2H-pyran-2-yl)methanone (673 mg) was prepared as described for ((S)-1-(4-fluorophenyl)-3,4-dihydroisoquinolin-2(1H)-yl)((S)-5-nitro-3,4-dihydro-2H-pyran-2-yl)methanone (see COMPOUND 5213). White solid.

LCMS: 68%, RT=2.11 min., (M+H)⁺=383 (method A).

### Synthesis of ((S)-1-(4-fluorophenyl)-3,4-dihydroisoquinolin-2(1H)-yl)((2R,5S)-5-nitrotetrahydro-2H-pyran-2-yl)methanone and ((S)-1-(4-fluorophenyl)-3,4-dihydroisoquinolin-2(1H)-yl)((2R,5R)-5-nitrotetrahydro-2H-pyran-2-yl)methanone

Starting from ((S)-1-(4-fluorophenyl)-3,4-dihydroisoquinolin-2(1H)-yl)((R)-5-nitro-3,4-dihydro-2H-pyran-2-yl)methanone (673 mg, 1.760 mmol), ((S)-1-(4-fluorophenyl)-3,4-dihydroisoquinolin-2(1H)-yl)((2R,5S)-5-nitrotetrahydro-2H-pyran-2-yl)methanone as the first eluting isomer on silica (0-100% ethyl acetate in heptane) and ((S)-1-(4-fluorophenyl)-3,4-dihydroisoquinolin-2(1H)-yl)((2R,5R)-5-nitrotetrahydro-2H-pyran-2-yl)methanone as the second eluting isomer on silica (0-100% ethylacetate in heptane) were prepared as described for ((S)-1-(4-fluorophenyl)-3,4-dihydroisoquinolin-2(1H)-yl)((2S,5R)-5-nitrotetrahydro-2H-pyran-2-yl)methanone (see COMPOUND 5213). Stereochemistry of the 5-nitro-pyran is arbitrarily assigned.

First eluting isomer: LCMS: 99%, RT=2.11 min., (M+H)⁺=385 (method A).

Second eluting isomer: LCMS: 99%, RT=2.07 min., (M+H)⁺=385 (method A).

### Synthesis of ((S)-1-(4-fluorophenyl)-3,4-dihydroisoquinolin-2(1H)-yl)((2R,5R)-5-(hydroxymethyl)-5-nitrotetrahydro-2H-pyran-2-yl)methanone and ((S)-1-(4-fluorophenyl)-3,4-dihydroisoquinolin-2(1H)-yl)((2R,5S)-5-(hydroxymethyl)-5-nitrotetrahydro-2H-pyran-2-yl)methanone

Starting from a mixture of, ((S)-1-(4-fluorophenyl)-3,4-dihydroisoquinolin-2(1H)-yl)((2R,5S)-5-nitrotetrahydro-2H-pyran-2-yl)methanone (139 mg, 0.362 mmol) and ((S)-1-(4-fluorophenyl)-3,4-dihydroisoquinolin-2(1H)-yl)((2R,5R)-5-nitrotetrahydro-2H-pyran-2-yl)methanone (207 mg, 0.538 mmol), ((S)-1-(4-fluorophenyl)-3,4-dihydroisoquinolin-2(1H)-yl)((2R,5R)-5-(hydroxymethyl)-5-nitrotetrahydro-2H-pyran-2-yl)methanone as the first eluting isomer on silica (0-100% ethyl acetate in heptane) and ((S)-1-(4-fluorophenyl)-3,4-dihydroisoquinolin-2(1H)-yl)((2R,5S)-5-(hydroxymethyl)-5-nitrotetrahydro-2H-pyran-2-yl)methanone as the second eluting isomer on silica (0-100% ethyl acetate in heptane) were prepared as described for ((S)-1-(4-fluorophenyl)-3,4-dihydroisoquinolin-2(1H)-yl)((2S,5R)-5-(hydroxymethyl)-5-nitrotetrahydro-2H-pyran-2-yl)methanone (see COMPOUND 5213). Stereochemistry of the 5-nitro-pyran is arbitrarily assigned.

First eluting isomer: LCMS: 99%, RT=2.03 min., (M+H)⁺=415 (method A).

Second eluting isomer: LCMS: 99%, RT=1.99 min., (M+H)⁺=415 (method A).

### Synthesis of ((2R,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5215)

Starting from ((S)-1-(4-fluorophenyl)-3,4-dihydroisoquinolin-2(1H)-yl)((2R,5R)-5-(hydroxymethyl)-5-nitrotetrahydro-2H-pyran-2-yl)methanone (51.1 mg, 0.123 mmol), ((2R,5R)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5215) was prepared as described for ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5213). Stereochemistry of the 5-amino-pyran is arbitrarily assigned

LCMS: 99%, RT=1.02 min., (M+H)⁺=385 (method P). SFC: 99%, RT=4.21 min., (M+H)⁺=385 (method F).

### Synthesis of ((2R,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5216)

Starting from ((S)-1-(4-fluorophenyl)-3,4-dihydroisoquinolin-2(1H)-yl)((2R,5S)-5-(hydroxymethyl)-5-nitrotetrahydro-2H-pyran-2-yl)methanone (51.9 mg, 0.125 mmol), ((2R,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5216) was prepared as described for ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (COMPOUND 5213). Stereochemistry of the 5-amino-pyran is arbitrarily assigned

LCMS: 99%, RT=1.02 min., (M+H)⁺=385 (method P, 40° C.). SFC: 99%, RT=4.45 min., (M+H)⁺=385 (method F).

### Example 82

### ((2R,4S,5S)-5-amino-4-fluorotetrahydro-2H-pyran-2-yl)((S)-1-(4-fluorophenyl)-3,4-dihydroisoquinolin-2(1/H)-yl)methanone (COMPOUND 5210)

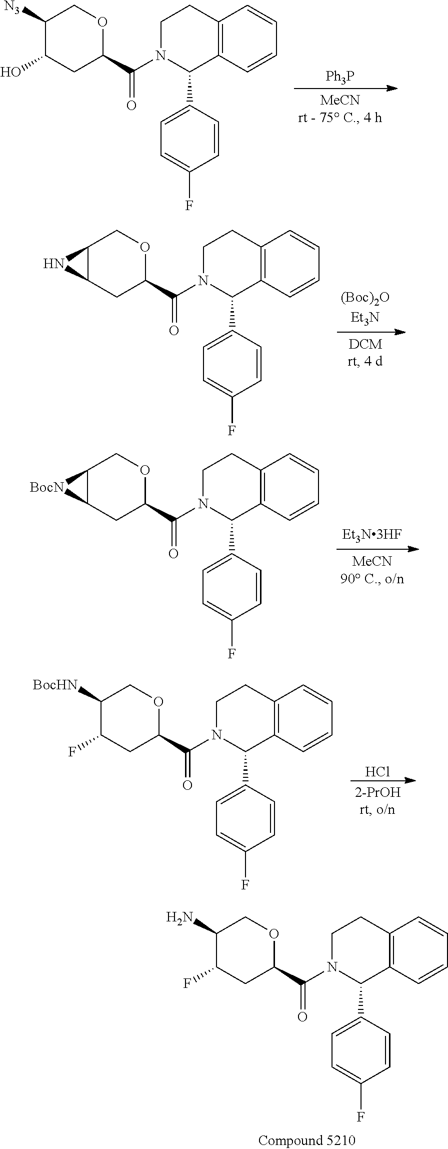

### Synthesis of ((1R,4R,6R)-3-oxa-7-azabicyclo[4.1.0]heptan-4-yl)((S)-1-(4-fluorophenyl)-3,4-dihydroisoquinolin-2(1H)-yl)methanone

Under nitrogen atmosphere, triphenylphosphine (119 mg, 0.454 mmol) was added to a suspension of ((2R,4S,5S)-5-azido-4-hydroxytetrahydro-2H-pyran-2-yl)((S)-1-(4-fluorophenyl)-3,4-dihydroisoquinolin-2(1H)-yl)methanone (150 mg, 0.378 mmol) in acetonitrile (dry, 3.8 mL). After 30 minutes, the mixture was warmed to 75° C. and stirred for 3.5 hours. Then, the reaction mixture was concentrated to dryness under reduced pressure. The residue was purified by flash column chromatography (silica, 0 to 80% ethyl acetate in heptane) and acidic preparative MPLC (Linear Gradient: t=0 min 5% A; t=1 min 5% A; t=17 min 50% A; t=18 min 100%; t=23 min 100% A; detection: 220 nm) to give ((1R,4R,6R)-3-oxa-7-azabicyclo[4.1.0]heptan-4-yl)((S)-1-(4-fluorophenyl)-3,4-dihydroisoquinolin-2(1H)-yl)methanone.

LCMS: 99%, RT=1.61 min., (M+H)⁺=353 (method A).

### Synthesis of tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate

Triethylamine (0.022 mL, 0.160 mmol) and di-tert-butyl dicarbonate (34.9 mg, 0.160 mmol) were added to a solution of ((1R,4R,6R)-3-oxa-7-azabicyclo[4.1.0]heptan-4-yl)((S)-1-(4-fluorophenyl)-3,4-dihydroisoquinolin-2(1H)-yl)methanone (47.0 mg, 0.133 mmol) in dichloromethane (0.5 mL). After 4 days, additional triethylamine (0.022 mL, 0.160 mmol) was added and stirring was continued for 2.5 hours. Then, the mixture was purified by flash column chromatography (silica, 0 to 45% ethyl acetate in heptane) to give tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate.

LCMS: 19%, RT=2.16 min., (M+H)⁺=453 and 81%, RT=2.06 min., (M+H)⁺=471 (product as hydrate) (method A).

### Synthesis of tert-butyl ((3S,4S,6R)-4-fluoro-6-((S)-1-(4-fluorophenyl)-1,2,3,4-tetrahydroisoquinoline-2-carbonyl)tetrahydro-2H-pyran-3-yl)carbamate

Under argon atmosphere in a microwave vial sealed with a septum, triethylamine trihydrofluoride (144 μL, 0.884 mmol) was added to a solution of tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate (50.0 mg, 0.110 mmol) in acetonitrile (dry, 0.50 mL). The mixture was placed in a preheated sand bath to 90° C. and stirred overnight. After cooling to room temperature, the mixture was partitioned between ethyl acetate (5 mL) and a mixture of brine and saturated aqueous NaHCO₃ (1:1, 3 mL). The layers were separated and the aqueous phase was extracted with ethyl acetate (2×2.5 mL). The combined organic layers were dried over Na₂SO₄ and evaporated under reduced pressure. The residue was purified by flash column chromatography (silica, 5 to 35% ethyl acetate in heptane) and preparative SFC (method V) to give tert-butyl ((3S,4S,6R)-4-fluoro-6-((S)-1-(4-fluorophenyl)-1,2,3,4-tetrahydroisoquinoline-2-carbonyl)tetrahydro-2H-pyran-3-yl)carbamate.

LCMS: 99%, RT=2.33 min., (M+H)⁺=473 (method A=scan acid). SFC: 99%, RT=1.89 min., (M+H)⁺=473 (method BR).

### Synthesis of ((2R,4S,5S)-5-amino-4-fluorotetrahydro-2H-pyran-2-yl)((S)-1-(4-fluorophenyl)-3,4-dihydroisoquinolin-2(1H)-yl)methanone (COMPOUND 5210)

HCl (5-6 M in 2-propanol, 339 μL, 1.862 mmol) was added to a solution of tert-butyl ((3S,4S,6R)-4-fluoro-6-((S)-1-(4-fluorophenyl)-1,2,3,4-tetrahydroisoquinoline-2-carbonyl)tetrahydro-2H-pyran-3-yl)carbamate (29.2 mg, 0.062 mmol) in 2-propanol (0.62 mL). After stirring overnight, the mixture was

diluted with dichloromethane (5 mL) and washed with saturated aqueous Na₂CO₃ (2 mL). The layers were separated and the aqueous phase was extracted with dichloromethane (2 mL). The combined organics were dried over Na₂SO₄ and evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilised from a mixture of acetonitrile and water (1:3, 2 mL) to give ((2R,4S,5S)-5-amino-4-fluorotetrahydro-2H-pyran-2-yl)((S)-1-(4-fluorophenyl)-3,4-dihydroisoquinolin-2(1H)-yl)methanone.

LCMS: 99%, RT=1.07 min., (M+H)⁺=373 (method P). SFC: 99%, RT=3.21 min., (M+H)⁺=373 (method W).

### Example 83

### ((2R,4R,5R)-4-amino-5-ethoxytetrahydro-2H-pyran-2-yl)((S)-1-(4-fluorophenyl)-3,4-dihydroisoquinolin-2(1H)-yl)methanone (COMPOUND 5248)

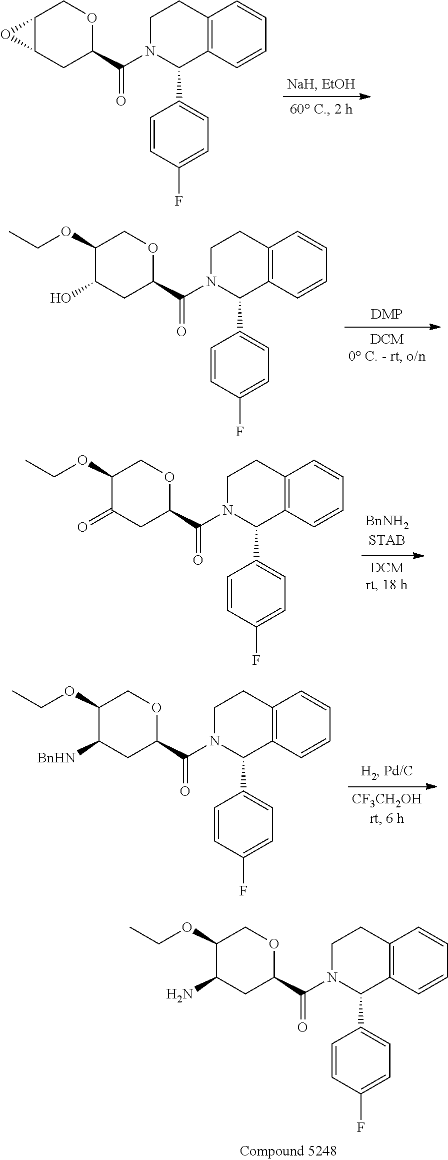

### Synthesis of ((2R,4S,5S)-5-ethoxy-4-hydroxytetrahydro-2H-pyran-2-yl)((S)-1-(4-fluorophenyl)-3,4-dihydroisoquinolin-2(1H)-yl)methanone

Sodium hydride (60% in mineral oil, 24.2 mg, 0.604 mmol) was added to ethanol (dry, 1.2 mL). After the foaming had ceased, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 1.0 g, 2.83 mmol) was added and the mixture was warmed to 60° C. for 2 hours. After cooling to room temperature, the mixture was diluted with dichloromethane (10 mL) and washed with saturated aqueous NH₄Cl (2 mL). The aqueous phase was extracted with dichloromethane (5 mL). The combined organics were dried over Na₂SO₄ and evaporated under reduced pressure. The residue was purified by flash column chromatography (silica, 40 to 80% ethyl acetate in heptane) and preparative SFC (method AW) to give ((2R,4S,5S)-5-ethoxy-4-hydroxytetrahydro-2H-pyran-2-yl)((S)-1-(4-fluorophenyl)-3,4-dihydroisoquinolin-2(1H)-yl)methanone.

LCMS: 99%, RT=1.98 min., (M+H)⁺=400 (method A), SFC: 99%, RT=4.79 min., (M+H)⁺=400 (method W).

### Synthesis of (2R,5S)-5-ethoxy-2-((S)-1-(4-fluorophenyl)-1,2,3,4-tetrahydroisoquinoline-2-carbonyl)tetrahydro-4H-pyran-4-one

At 0° C., Dess-Martin periodinane (111 mg, 0.262 mmol) was added to a solution of ((2R,4S,5S)-5-ethoxy-4-hydroxytetrahydro-2H-pyran-2-yl)((S)-1-(4-fluorophenyl)-3,4-dihydroisoquinolin-2(1H)-yl)methanone (95 mg, 0.238 mmol) in dichloromethane (dry, 2.4 mL). The mixture was allowed to warm to room temperature and stirred overnight. The mixture was diluted with a mixture of saturated aqueous NaHCO₃ and saturated aqueous Na₂S₂O₃ (1:1, 2 mL) and stirred vigorously for 15 minutes. Then, the mixture was further diluted with dichloromethane (4 mL) and a mixture of saturated aqueous NaHCO₃ and saturated aqueous Na₂S₂O₃ (1:1, 2 mL). The layers were separated and the aqueous phase was extracted with dichloromethane (4 mL). The combined organics were dried over Na₂SO₄ and evaporated under reduced pressure. The residue was purified by flash column chromatography (silica, 20 to 60% ethyl acetate in heptane) to give (2R,5S)-5-ethoxy-2-((S)-1-(4-fluorophenyl)-1,2,3,4-tetrahydroisoquinoline-2-carbonyl)tetrahydro-4H-pyran-4-one.

LCMS: 99%, plateau between two peaks at RT=1.86 and 2.04 min., (M+H)⁺=398 (method A).

### Synthesis of ((2R,4R,5R)-4-(benzylamino)-5-ethoxytetrahydro-2H-pyran-2-yl)((S)-1-(4-fluorophenyl)-3,4-dihydroisoquinolin-2(1H)-yl)methanone

Under nitrogen atmosphere, benzylamine (0.047 mL, 0.428 mmol) was added to a solution of (2R,5S)-5-ethoxy-2-((S)-1-(4-fluorophenyl)-1,2,3,4-tetrahydroisoquinoline-2-carbonyl)tetrahydro-4H-pyran-4-one (85.0 mg, 0.214 mmol) in dichloromethane (dry, 2.1 mL). After 35 minutes, sodium triacetoxyborohydride (113 mg, 0.535 mmol) was added. After stirring overnight, the mixture was diluted with dichloromethane (3 mL) and washed with saturated aqueous NaHCO₃ (2 mL). The aqueous layer was extracted with dichloromethane (3 mL) and the combined organic layers were dried over Na₂SO₄ and evaporated under reduced pressure. The residue was purified by flash column chromatography (silica, 1 to 3.5% (7M ammonia in methanol) in dichloromethane) to give ((2R,4S,5R)-4-(benzylamino)-5-ethoxytetrahydro-2H-pyran-2-yl)((S)-1-(4-fluorophenyl)-3,4-dihydroisoquinolin-2(1H)-yl)methanone (22.5 mg, 22%) as the first eluting isomer as a yellow solid and ((2R,4R,5R)-4-(benzylamino)-5-ethoxytetrahydro-2H-pyran-2-yl)((S)-1-(4-fluorophenyl)-3,4-dihydroisoquinolin-2(1H)-yl)methanone (71.8 mg) as the second eluting isomer as an off-white solid.

First eluting isomer: LCMS: 93%, RT=1.92 min., (M+H)⁺=489 (method A). SFC: 99% (d.e.), RT=1.75 min., (M+H)⁺=489 (method AS).

Second eluting isomer: LCMS: 99%, RT=1.89 min., (M+H)⁺=489 (method A). SFC: 99%, RT=2.36 min., (M+H)⁺=489 (method AS).

### Synthesis of ((2R,4R,5R)-4-amino-5-ethoxytetrahydro-2H-pyran-2-yl)((S)-1-(4-fluorophenyl)-3,4-dihydroisoquinolin-2(1H)-yl)methanone (COMPOUND 5248)

A solution of ((2R,4R,5R)-4-(benzylamino)-5-ethoxytetrahydro-2H-pyran-2-yl)((S)-1-(4-fluorophenyl)-3,4-dihydroisoquinolin-2(1H)-yl)methanone (69 mg, 0.141 mmol) in 2,2,2-trifluoroethanol (4.0 mL) was hydrogenated in the presence of palladium (10 wt % on carbon, containing 50% water, 45.1 mg, 0.021 mmol) and 5.5 bar hydrogen pressure. After 6 hours, the reaction mixture was filtered and evaporated under reduced pressure. The residue was purified by flash column chromatography (silica, 1 to 6% (7M ammonia in methanol) in dichloromethane) and preparative SFC (method BS) to give ((2R,4R,5R)-4-amino-5-ethoxytetrahydro-2H-pyran-2-yl)((S)-1-(4-fluorophenyl)-3,4-dihydroisoquinolin-2(1H)-yl)methanone (COMPOUND 5248).

LCMS: 99%, RT=1.09 min., (M+H)⁺=399 (method P). SFC: 99%, RT=2.72 min., (M+H)⁺=399 (method AS).

### Example 84

### ((2R,4S,5S)-4-(dimethylamino)-5-hydroxytetrahydro-2H-pyran-2-yl)((S)-1-(4-fluorophenyl)-3,4-dihydroisoquinolin-2(1H)-yl)methanone (COMPOUND 5447)

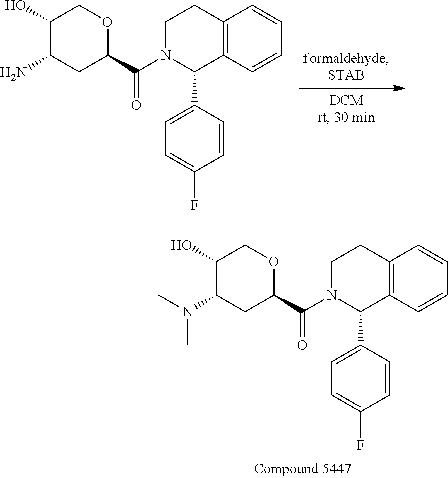

### Synthesis of ((2R,4S,5S)-4-(dimethylamino)-5-hydroxytetrahydro-2H-pyran-2-yl)((S)-1-(4-fluorophenyl)-3,4-dihydroisoquinolin-2(1H)-yl)methanone

Formaldehyde (37% wt solution in H₂O (stabilized with 7-8% MeOH), 0.016 mL, 0.213 mmol) and sodium triacetoxyborohydride (45.2 mg, 0.213 mmol) were added to a solution of ((2R,4S,5S)-4-amino-5-hydroxytetrahydro-2H-pyran-2-yl)((S)-1-(4-fluorophenyl)-3,4-dihydroisoquinolin-2(1H)-yl)methanone (see Compound 5051, 79 mg, 0.213 mmol) in dichloromethane (1 mL). After 10 minutes, additional formaldehyde (37% wt solution in H₂O (stabilized with 7-8% MeOH), 0.226 mL, 0.427 mmol) and sodium triacetoxyborohydride (181 mg, 0.853 mmol) were added. The reaction mixture was stirred for 30 minutes, diluted with dichloromethane (5 mL) and quenched with saturated aqueous NaHCO₃ (5 mL). The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilised from a mixture of acetonitrile and water (1:2, 3 mL) to give ((2R,4S,5S)-4-(dimethylamino)-5-hydroxytetrahydro-2H-pyran-2-yl)((S)-1-(4-fluorophenyl)-3,4-dihydroisoquinolin-2(1H)-yl)methanone.

LCMS: 99%, RT=1.12 min., (M+H)⁺=399 (method P).

### Example 85

### ((2R,4R,5R)-4-amino-5-fluorotetrahydro-2H-pyran-2-yl)((S)-1-(4-fluorophenyl)-3,4-dihydroisoquinolin-2(1H)-yl)methanone (COMPOUND 5250)

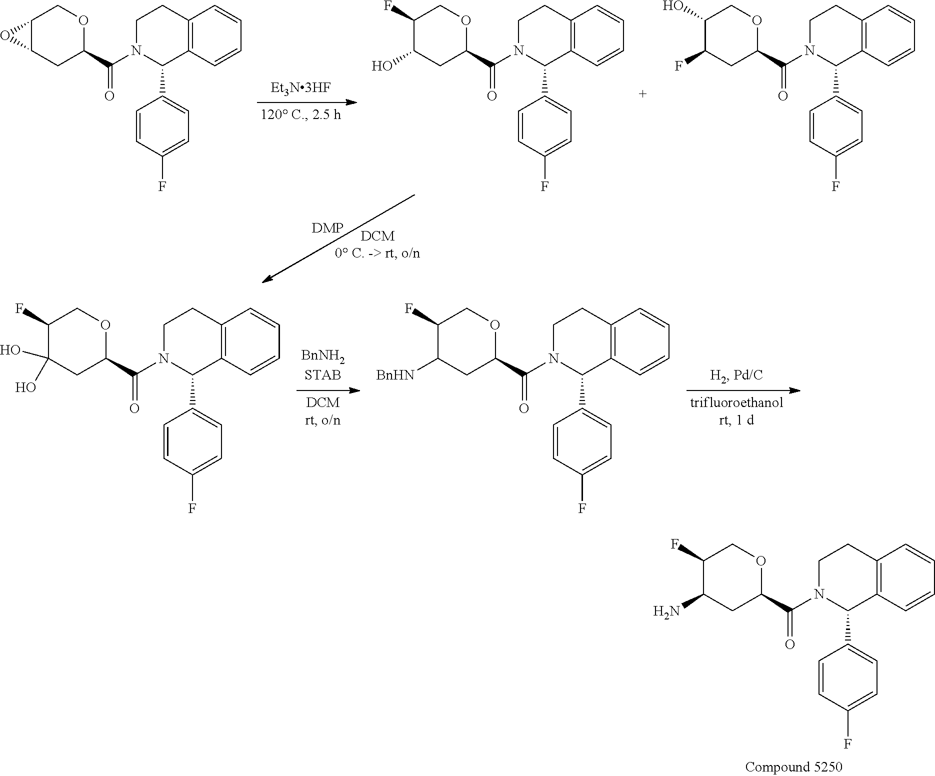

### Synthesis of ((2R,4S,5S)-5-fluoro-4-hydroxytetrahydro-2H-pyran-2-yl)((S)-1-(4-fluorophenyl)-3,4-dihydroisoquinolin-2(1H)-yl)methanone

Under argon atmosphere in a microwave vial, triethylamine trihydrofluoride (2.076 mL, 12.73 mmol) was added to ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 1.0 g, 2.83 mmol). The vial was capped and placed into a pre-heated sand bath on 120° C. After 2.5 hours, the mixture was cooled to room temperature, diluted with dichloromethane (10 mL), and poured into a mixture of ice and saturated aqueous Na₂CO₃ (1:2, 30 mL). Once gas evolution ceased, the mixture was further diluted with dichloromethane (30 mL) and the layers were separated. The aqueous phase was extracted with dichloromethane (20 mL) and the combined organic layers were washed with aqueous citric acid (0.5M, 20 mL) and a mixture of aqueous citric acid (0.5M) and brine (1:1, 40 mL), dried over Na₂SO₄, and evaporated under reduced pressure. The residue was purified by flash column chromatography (silica, 30 to 50% ethyl acetate in heptane) to give ((2R,4S,5S)-5-fluoro-4-hydroxytetrahydro-2H-pyran-2-yl)((S)-1-(4-fluorophenyl)-3,4-dihydroisoquinolin-2(1H)-yl)methanone as the first eluting isomer after crystallization from methanol and ((2R,4R,5R)-5-fluoro-4-hydroxytetrahydro-2H-pyran-2-yl)((S)-1-(4-fluorophenyl)-3,4-dihydroisoquinolin-2(1H)-yl)methanone as the second eluting isomer.

First eluting isomer: LCMS: 100%, RT=1.94 min., (M+H)⁺=374 (method A). SFC: 98%, RT=2.34 min., (M+H)⁺=374 (method V).

Second eluting isomer: LCMS: 100%, RT=2.09 min., (M+H)⁺=374 (method A). SFC: 98%, RT=2.50 min., (M+H)⁺=374 (method V).

### Synthesis of ((2R,5S)-5-fluoro-4,4-dihydroxytetrahydro-2H-pyran-2-yl)((S)-1-(4-fluorophenyl)-3,4-dihydroisoquinolin-2(1H)-yl)methanone

At 0° C., Dess-Martin periodinane (210 mg, 0.495 mmol) was added to a solution of ((2R,4S,5S)-5-fluoro-4-hydroxytetrahydro-2H-pyran-2-yl)((S)-1-(4-fluorophenyl)-3,4-dihydroisoquinolin-2(1H)-yl)methanone (168 mg, 0.450 mmol) in dichloromethane (dry, 4.5 mL). The reaction mixture was allowed to warm to room temperature and stirred overnight. Then, the mixture was diluted with a mixture saturated aqueous NaHCO₃ and saturated aqueous Na₂S₂O₃ (1:1, 4 mL) and stirred vigorously. After 15 minutes, the mixture was diluted with dichloromethane (10 mL) and a mixture saturated aqueous NaHCO₃ and saturated aqueous Na₂S₂O₃ (1:1, 4 mL) and the layers were separated. The aqueous layer was extracted with dichloromethane (10 mL) and the combined organic layers were dried over Na₂SO₄ and evaporated under reduced pressure. The residue was purified by flash column chromatography (silica, 35 to 95% ethyl acetate in heptane) to give ((2R,5S)-5-fluoro-4,4-dihydroxytetrahydro-2H-pyran-2-yl)((S)-1-(4-fluorophenyl)-3,4-dihydroisoquinolin-2(1H)-yl)methanone.

LCMS: 99%, RT=2.03 min., (M+H)⁺=390 (method A).

### Synthesis of ((2R,4R,5R)-4-(benzylamino)-5-fluorotetrahydro-2H-pyran-2-yl)((S)-1-(4-fluorophenyl)-3,4-dihydroisoquinolin-2(1H)-yl)methanone

Under nitrogen atmosphere, benzylamine (0.082 mL, 0.749 mmol) was added to a solution of (2R,5S)-5-fluoro-2-((S)-1-(4-fluorophenyl)-1,2,3,4-tetrahydroisoquinoline-2-carbonyl)tetrahydro-4H-pyran-4-one (139 mg, 0.374 mmol) in dichloromethane (dry, 3.7 mL). After 35 minutes, sodium triacetoxyborohydride (198 mg, 0.936 mmol) was added and the resulting suspension was stirred overnight. The mixture was diluted with dichloromethane (6 mL) and washed with saturated aqueous NaHCO₃ (4 mL). The aqueous layer was extracted with dichloromethane (10 mL) and the combined organic layers were dried over Na₂SO₄ and evaporated under reduced pressure. The residue was purified by flash column chromatography (silica, 1 to 6% (7M ammonia in methanol) in dichloromethane) and acidic preparative MPLC (Linear Gradient: t=0 min 5% A; t=1 min 5% A; t=2 min 10% A; t=17 min 50% A; t=18 min 100%; t=23 min 100% A; detection: 210 nm). The product fractions were combined, diluted with a mixture of brine and saturated aqueous Na₂CO₃ (1:1, 10 mL), and extracted with dichloromethane (3×20 mL). The combined organics were dried over Na₂SO₄ and evaporated under reduced pressure to give ((2R,4R,5R)-4-(benzylamino)-5-fluorotetrahydro-2H-pyran-2-yl)((S)-1-(4-fluorophenyl)-3,4-dihydroisoquinolin-2(1H)-yl)methanone.

LCMS: 99%, RT=1.72 min., (M+H)⁺=463 (method A). SFC: 99%, RT=4.62 min., (M+H)⁺=463 (method F).

### Synthesis of ((2R,4R,5R)-4-amino-5-fluorotetrahydro-2H-pyran-2-yl)((S)-1-(4-fluorophenyl)-3,4-dihydroisoquinolin-2(1H)-yl)methanone (COMPOUND 5250)

A solution of ((2R,4R,5R)-4-(benzylamino)-5-fluorotetrahydro-2H-pyran-2-yl)((S)-1-(4-fluorophenyl)-3,4-dihydroisoquinolin-2(1H)-yl)methanone (75 mg, 0.162 mmol) in 2,2,2-trifluoroethanol (4.0 mL) was hydrogenated in the presence of palladium (10 wt % on carbon, containing 50% water, 51.8 mg, 0.024 mmol) and 5.5 bar hydrogen pressure. After 1 day, the reaction mixture was filtered and evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:2, 3 mL) to give ((2R,4R,5R)-4-amino-5-fluorotetrahydro-2H-pyran-2-yl)((S)-1-(4-fluorophenyl)-3,4-dihydroisoquinolin-2(1H)-yl)methanone (COMPOUND 5250).

LCMS: 99%, RT=1.04 min., (M+H)⁺=373 (method P).

### Example 86

### ((2R,4S,5R)-4-amino-5-(2,2,2-trifluoroethoxy)tetrahydro-2H-pyran-2-yl)((S)-1-(4-fluorophenyl)-3,4-dihydroisoquinolin-2(1H)-yl)methanone (COMPOUND 5475)

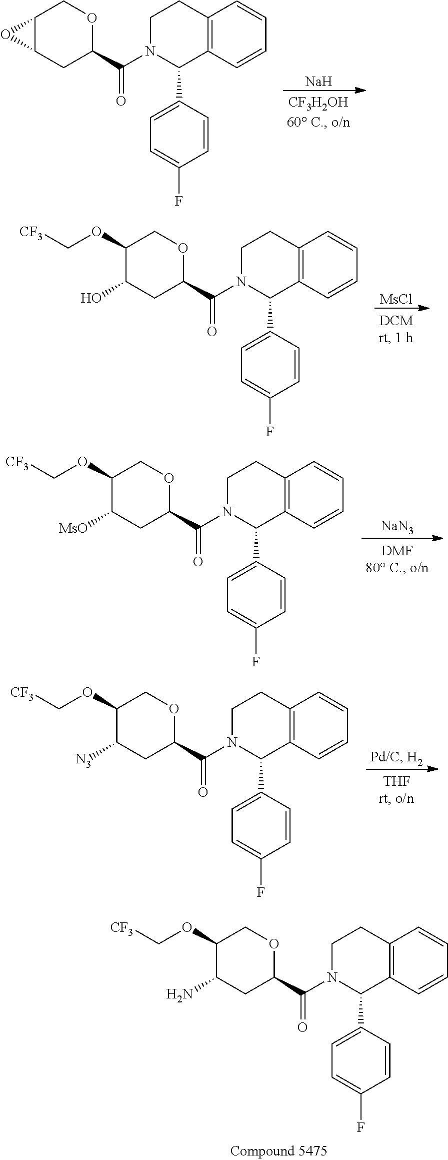

### Synthesis of ((S)-1-(4-fluorophenyl)-3,4-dihydroisoquinolin-2(1H)-yl)((2R,4S,5S)-4-hydroxy-5-(2,2,2-trifluoroethoxy)tetrahydro-2H-pyran-2-yl)methanone

Under nitrogen atmosphere, sodium hydride (60 wt % dispersion in oil, 56.6 mg, 1.415 mmol) was added to 2,2,2-trifluoroethanol (5 mL). Then ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 250 mg, 0.707 mmol) was added and the mixture was stirred at 60° C. overnight. The mixture was diluted with dichloromethane, washed with saturated aqueous NH₄Cl, dried over Na₂SO₄, and evaporated under reduced pressure. The residue was purified by flash column chromatography (silica, 40 to 100% ethyl acetate in heptane) and preparative SFC (method Z) to give ((S)-1-(4-fluorophenyl)-3,4-dihydroisoquinolin-2(1H)-yl)((2R,4S,5S)-4-hydroxy-5-(2,2,2-trifluoroethoxy)tetrahydro-2H-pyran-2-yl)methanone.

LCMS: 97%, RT=2.06 min., (M+H)⁺=454 (method A). SFC: 99%, RT=3.55 min., (M+H)⁺=454 (method Z).

### Synthesis of (2R,4S,5S)-2-((S)-1-(4-fluorophenyl)-1,2,3,4-tetrahydroisoquinoline-2-carbonyl)-5-(2,2,2-trifluoroethoxy)tetrahydro-2H-pyran-4-yl methanesulfonate

Triethylamine (0.069 mL, 0.496 mmol) and methanesulfonyl chloride (0.026 mL, 0.331 mmol) were added to a solution of ((S)-1-(4-fluorophenyl)-3,4-dihydroisoquinolin-2(1H)-yl)((2R,4S,5S)-4-hydroxy-5-propoxytetrahydro-2H-pyran-2-yl)methanone (75 mg, 0.165 mmol) in dichloromethane (5 mL). After 1 hour, the mixture was washed with aqueous citric acid (0.5M) and saturated aqueous NaHCO₃, passed through a phase separator, and evaporated under reduced pressure to give (2R,4S,5S)-2-((S)-1-(4-fluorophenyl)-1,2,3,4-tetrahydroisoquinoline-2-carbonyl)-5-(2,2,2-trifluoroethoxy)tetrahydro-2H-pyran-4-yl methanesulfonate.

LCMS: 99%, RT=1.91 min., (M+H)⁺=532 (method P).

### Synthesis of ((2R,4S,5R)-4-azido-5-(2,2,2-trifluoroethoxy)tetrahydro-2H-pyran-2-yl)((S)-1-(4-fluorophenyl)-3,4-dihydroisoquinolin-2(1H)-yl)methanone

Sodium azide (33.0 mg, 0.508 mmol) was added to a solution of (2R,4S,5S)-2-((S)-1-(4-fluorophenyl)-1,2,3,4-tetrahydroisoquinoline-2-carbonyl)-5-(2,2,2-trifluoroethoxy)tetrahydro-2H-pyran-4-yl methanesulfonate (0.165 mmol) in N,N-dimethylformamide (6 mL) and the mixture was heated to 80° C. After stirring overnight, the reaction mixture was diluted with water and extracted with ethyl acetate (3×). The combined organics were dried over Na₂SO₄ and evaporated under reduced pressure. The residue was purified by flash column chromatography (silica, 0 to 100% ethyl acetate in heptane) to give ((2R,4S,5R)-4-azido-5-(2,2,2-trifluoroethoxy)tetrahydro-2H-pyran-2-yl)((S)-1-(4-fluorophenyl)-3,4-dihydroisoquinolin-2(1H)-yl)methanone.

LCMS: 96%, RT=2.24 min., (M+H)⁺=479 (method A).

### Synthesis of ((2R,4S,5R)-4-amino-5-(2,2,2-trifluoroethoxy)tetrahydro-2H-pyran-2-yl)((S)-1-(4-fluorophenyl)-3,4-dihydroisoquinolin-2(1H)-yl)methanone (COMPOUND 5475)

A solution of ((2R,4S,5R)-4-azido-5-(2,2,2-trifluoroethoxy)tetrahydro-2H-pyran-2-yl)((S)-1-(4-fluorophenyl)-3,4-dihydroisoquinolin-2(1H)-yl)methanone (46 mg, 0.096 mmol) in tetrahydrofuran (5 mL) was hydrogenated in the presence of palladium (10 wt % on carbon, containing 50% water, 20.46 mg, 0.019 mmol) and atmospheric hydrogen pressure. After overnight stirring, the reaction mixture was filtered and evaporated under reduced pressure. The residue was dissolved in methanol and brought onto an SCX-2 column (1 g) and eluted with methanol until neutral. Next, the column was eluted with ammonia in methanol (2 M). The basic fraction was concentrated to dryness under reduced pressure. The residue was lyophilized from a mixture of acetonitrile and water (1:1, 4 mL) to give ((2R,4S,5R)-4-amino-5-(2,2,2-trifluoroethoxy)tetrahydro-2H-pyran-2-yl)((S)-1-(4-fluorophenyl)-3,4-dihydroisoquinolin-2(1H)-yl)methanone (COMPOUND 5475).

LCMS: 99%, RT=1.20 min., (M+H)⁺=453 (method P).

### Example 87

### ((2R,4S,5R)-4-amino-5-propoxytetrahydro-2H-pyran-2-yl)((S)-1-(4-fluorophenyl)-3,4-dihydroisoquinolin-2(1H)-yl)methanone (COMPOUND 5502)

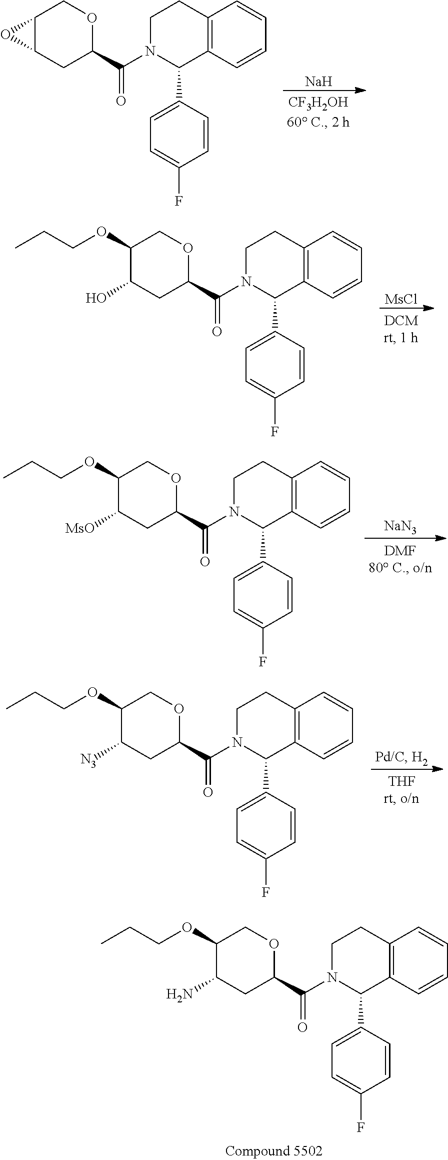

### Synthesis of ((S)-1-(4-fluorophenyl)-3,4-dihydroisoquinolin-2(1H)-yl)((2R,4S,5S)-4-hydroxy-5-propoxytetrahydro-2H-pyran-2-yl)methanone

Starting from ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 240 mg, 0.679 mmol) and 1-propanol (5 mL), ((S)-1-(4-fluorophenyl)-3,4-dihydroisoquinolin-2(1H)-yl)((2R,4S,5S)-4-hydroxy-5-propoxytetrahydro-2H-pyran-2-yl)methanone was prepared as described for ((S)-1-(4-fluorophenyl)-3,4-dihydroisoquinolin-2(1H)-yl)((2R,4S,5S)-4-hydroxy-5-(2,2,2-trifluoroethoxy)tetrahydro-2H-pyran-2-yl)methanone (see Compound 5475).

LCMS: 99%, RT=2.03 min., (M+H)⁺=414 (method A). SFC: 99%, RT=4.53 min., (M+H)⁺=414 (method Z).

### Synthesis of (2R,4S,5S)-2-((S)-1-(4-fluorophenyl)-1,2,3,4-tetrahydroisoquinoline-2-carbonyl)-5-propoxytetrahydro-2H-pyran-4-yl methanesulfonate

Starting from ((S)-1-(4-fluorophenyl)-3,4-dihydroisoquinolin-2(1H)-yl)((2R,4S,5S)-4-hydroxy-5-propoxytetrahydro-2H-pyran-2-yl)methanone (160 mg, 0.387 mmol), (2R,4S,5S)-2-((S)-1-(4-fluorophenyl)-1,2,3,4-tetrahydroisoquinoline-2-carbonyl)-5-propoxytetrahydro-2H-pyran-4-yl methanesulfonate (170 mg) was prepared as described for (2R,4S,5S)-2-((S)-1-(4-fluorophenyl)-1,2,3,4-tetrahydroisoquinoline-2-carbonyl)-5-(2,2,2-trifluoroethoxy)tetrahydro-2H-pyran-4-yl methanesulfonate (see Compound 5475).

LCMS: 98%, RT=2.18 min., (M+H)⁺=492 (method A).

### Synthesis of ((2R,4S,5R)-4-azido-5-propoxytetrahydro-2H-pyran-2-yl)((S)-1-(4-fluorophenyl)-3,4-dihydroisoquinolin-2(1H)-yl)methanone

Starting from (2R,4S,5S)-2-((S)-1-(4-fluorophenyl)-1,2,3,4-tetrahydroisoquinoline-2-carbonyl)-5-propoxytetrahydro-2H-pyran-4-yl methanesulfonate (170 mg, 0.346 mmol), ((2R,4S,5R)-4-azido-5-propoxytetrahydro-2H-pyran-2-yl)((S)-1-(4-fluorophenyl)-3,4-dihydroisoquinolin-2(1H)-yl)methanone was prepared as described for ((2R,4S,5R)-4-azido-5-(2,2,2-trifluoroethoxy)tetrahydro-2H-pyran-2-yl)((S)-1-(4-fluorophenyl)-3,4-dihydroisoquinolin-2(1H)-yl)methanone (see Compound 5475).

LCMS: 99%, RT=2.14 min., (M+H)⁺=439 (method P).

### Synthesis of ((2R,4S,5R)-4-amino-5-propoxytetrahydro-2H-pyran-2-yl)((S)-1-(4-fluorophenyl)-3,4-dihydroisoquinolin-2(1H)-yl)methanone (Compound 5502)

Starting from ((2R,4S,5R)-4-azido-5-propoxytetrahydro-2H-pyran-2-yl)((S)-1-(4-fluorophenyl)-3,4-dihydroisoquinolin-2(1H)-yl)methanone (120 mg, 0.274 mmol), ((2R,4S,5R)-4-amino-5-propoxytetrahydro-2H-pyran-2-yl)((S)-1-(4-fluorophenyl)-3,4-dihydroisoquinolin-2(1H)-yl)methanone (Compound 5502) was prepared as described for ((2R,4S,5R)-4-amino-5-(2,2,2-trifluoroethoxy)tetrahydro-2H-pyran-2-yl)((S)-1-(4-fluorophenyl)-3,4-dihydroisoquinolin-2(1H)-yl)methanone (Compound 5475).

LCMS: 95%, RT=1.20 min., (M+H)⁺=413 (method P).

### Example 88

### ((S)-1-(4-fluorophenyl)-3,4-dihydroisoquinolin-2(1H)-yl)((2R,4S,5R)-5-methoxy-4-(methylamino)tetrahydro-2H-pyran-2-yl)methanone (Compound 5503)

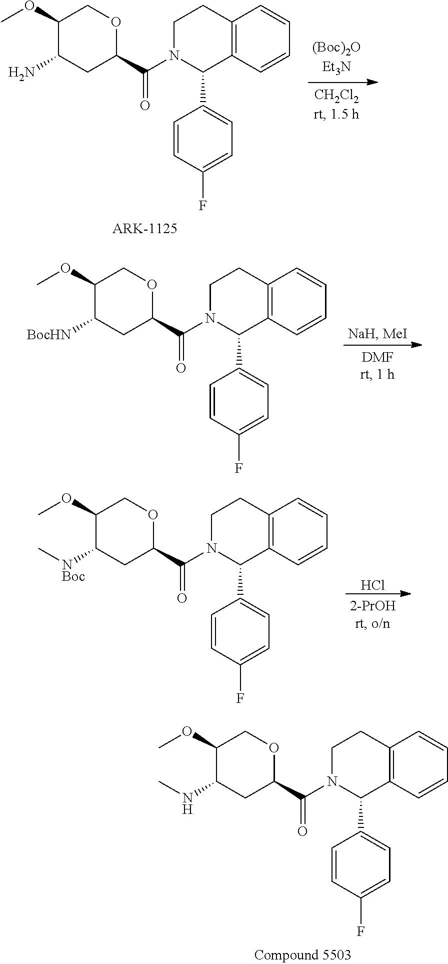

### Synthesis of tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)carbamate

Di-tert-butyl dicarbonate (36.2 mg, 0.166 mmol) was added to a solution of ((2R,4S,5R)-4-amino-5-methoxytetrahydro-2H-pyran-2-yl)((S)-1-(4-fluorophenyl)-3,4-dihydroisoquinolin-2(1H)-yl)methanone (Compound 5077, 53 mg, 0.138 mmol) and triethylamine (0.023 mL, 0.166 mmol) in dichloromethane. After 1.5 hour, the reaction mixture was concentrated to dryness under reduced pressure. The residue was purified by flash column chromatography (silica, 0 to 50% ethyl acetate in heptane) to give tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)carbamate.

LCMS: 100%, RT=2.13 min., (M+H)⁺=485 (method A).

### Synthesis of tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)(methyl)carbamate

Under nitrogen atmosphere, sodium hydride (60% dispersion in mineral oil, 3.96 mg, 0.099 mmol) was added to a solution of tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)carbamate (40.0 mg, 0.083 mmol) in N,N-dimethylformamide (dry, 0.415 mL). After 15 minutes, iodomethane (18 μL, 0.289 mmol) was added and the mixture was stirred for 50 minutes. Then, the mixture was diluted with ethyl acetate (5 mL) and washed with brine (2 mL). The aqueous layer was extracted with ethyl acetate (1 mL). The combined organic layers were washed with brine (4×2 mL), dried over Na₂SO₄, and evaporated under reduced pressure. The residue was purified by flash column chromatography (silica, 10 to 60% ethyl acetate in heptane) to give tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)(methyl)carbamate.

LCMS: 100%, RT=2.25 min., (M+H)⁺=499 (method A).

### Synthesis of ((S)-1-(4-fluorophenyl)-3,4-dihydroisoquinolin-2(1H)-yl)((2R,4S,5R)-5-methoxy-4-(methylamino)tetrahydro-2H-pyran-2-yl)methanone (Compound 5503)

HCl (5-6 M in 2-propanol), 0.354 mL, 1.949 mmol) was added dropwise to a solution of tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)(methyl)carbamate (32.4 mg, 0.065 mmol) in 2-propanol (0.650 mL) and the mixture was stirred overnight. The mixture was diluted with dichloromethane (4 mL) and washed with saturated aqueous Na₂CO₃ (1.5 mL). The aqueous phase was extracted with dichloromethane (2 mL) and the combined organics were dried over Na₂SO₄ and evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:2, 2 mL) to give ((S)-1-(4-fluorophenyl)-3,4-dihydroisoquinolin-2(1H)-yl)((2R,4S,5R)-5-methoxy-4-(methylamino)tetrahydro-2H-pyran-2-yl)methanone (Compound 5503). LCMS: 99%, RT=1.11 min., (M+H)⁺=399 (method P). SFC: 100%, RT=2.54 min., (M+H)⁺=399 (method AS).

### Example 89

### ((2R,4S,5R)-4-(ethylamino)-5-methoxytetrahydro-2H-pyran-2-yl)((S)-1-(4-fluorophenyl)-3,4-dihydroisoquinolin-2(1H)-yl)methanone (Compound 5504)

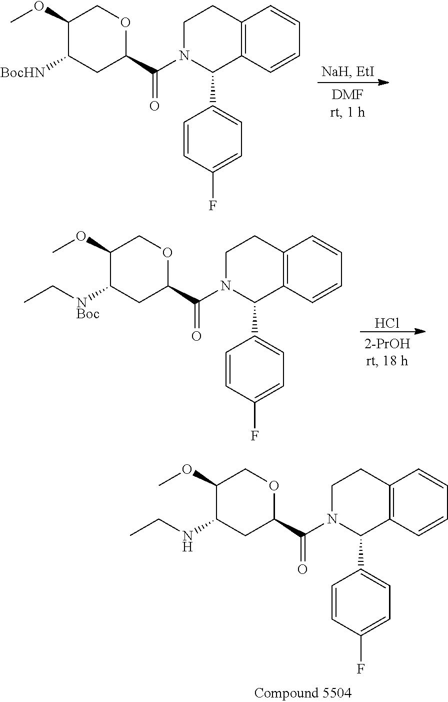

### Synthesis of tert-butyl ethyl((2R,4S,5R)-2-((S)-1-(4-fluorophenyl)-1,2,3,4-tetrahydroisoquinoline-2-carbonyl)-5-methoxytetrahydro-2H-pyran-4-yl)carbamate

Starting from tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)carbamate (see Compound 5503, 38.4 mg, 0.079 mmol) and ethyl iodide (22.2 μL, 0.277 mmol), tert-butyl ethyl((2R,4S,5R)-2-((S)-1-(4-fluorophenyl)-1,2,3,4-tetrahydroisoquinoline-2-carbonyl)-5-methoxytetrahydro-2H-pyran-4-yl)carbamate was prepared as described for tert-butyl ((2R,4S,5R)-2-((S)-1-(4-fluorophenyl)-1,2,3,4-tetrahydroisoquinoline-2-carbonyl)-5-methoxytetrahydro-2H-pyran-4-yl)(methyl)carbamate (see Compound 5503).

LCMS: 99%, RT=2.49 min., (M+H)⁺=513 (method A).

### Synthesis of ((2R,4S,5R)-4-(ethylamino)-5-methoxytetrahydro-2H-pyran-2-yl)((S)-1-(4-fluorophenyl)-3,4-dihydroisoquinolin-2(1H)-yl)methanone

Starting from tert-butyl ethyl((2R,4S,5R)-2-((S)-1-(4-fluorophenyl)-1,2,3,4-tetrahydroisoquinoline-2-carbonyl)-5-methoxytetrahydro-2H-pyran-4-yl)carbamate (31.8 mg, 0.062 mmol), ((2R,4S,5R)-4-(ethylamino)-5-methoxytetrahydro-2H-pyran-2-yl)((S)-1-(4-fluorophenyl)-3,4-dihydroisoquinolin-2(1H)-yl)methanone (Compound 5504) was prepared as described for ((S)-1-(4-fluorophenyl)-3,4-dihydroisoquinolin-2(1H)-yl)((2R,4S,5R)-5-methoxy-4-(methylamino)tetrahydro-2H-pyran-2-yl)methanone (Compound 5503).

LCMS: 98%, RT=1.14 min., (M+H)⁺=413 (method P). SFC: 99%, RT=2.33 min., (M+H)⁺=413 (method AU).

### Example 90

### ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (Compound 5217)

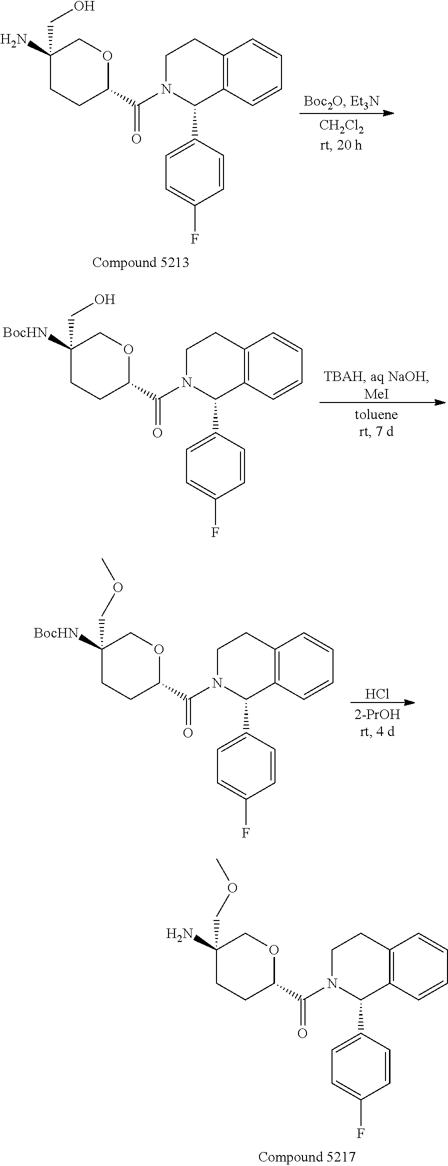

### Synthesis of tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate

Di-tert-butyl dicarbonate (47.5 mg, 0.218 mmol) was added to a solution of crude ((2S,5S)-5-amino-5-(hydroxymethyl)tetrahydro-2H-pyran-2-yl)((S)-1-(4-fluorophenyl)-3,4-dihydroisoquinolin-2(1H)-yl)methanone (Compound 5213, 0.145 mmol) in dichloromethane (4 mL). After 20 hours, the reaction mixture was concentrated to dryness under reduced pressure. The residue was purified by flash column chromatography (silica, 0 to 100% ethyl acetate in heptane) to give tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate.

LCMS: 95%, RT=2.05 min, (M+H)⁺=485 (method A).

### Synthesis of tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate

Sodium hydroxide (50 wt % solution in water, 0.063 mL, 1.67 mmol) and methyliodide (0.014 mL, 0.223 mmol) were added to a solution of tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate (27 mg, 0.056 mmol) and tetrabutylammonium hydrogensulfate (5.7 mg, 0.017 mmol) in toluene (2 mL). After 4 days, the reaction mixture was diluted with dichloromethane (5 mL) and water (1 mL). The layers were separated using a phase-separator and the organic filtrate was evaporated under reduced pressure. The residue was taken up into toluene (2 mL) and treated with tetrabutylammonium hydrogensulfate (11.4 mg, 0.033 mmol), sodium hydroxide (50 wt % solution in water, 0.126 mL, 3.34 mmol) and methyl iodide (0.035 mL, 0.557 mmol) for 3 days. The reaction mixture was diluted with dichloromethane (5 mL) and water (1 mL). The layers were separated using a phase-separator and the organic filtrate was evaporated under reduced pressure. The residue was purified by acidic preparative MPLC (Linear Gradient: t=0 min 5% A; t=1 min 30% A; t=16 min 70% A; t=17 min 100%; t=22 min 100% A; detection: 220/292 nm) to give tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (10.7 mg) as a white solid after lyophilization.

LCMS: 99%, RT=2.19 min., (M+H)⁺=499 (method A).

### Synthesis of ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (Compound 5217)

HCl (6 M in 2-propanol, 0.283 mL, 1.697 mmol) was added to a solution of tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (9 mg, 0.018 mmol) in 2-propanol (1 mL). After 4 days, the reaction mixture was diluted with dichloromethane (10 mL) and washed with aqueous saturated K₂CO₃ (3 mL). The organic layer was dried over Na₂SO₄ and evaporated under reduced pressure filtered. The residue was dissolved in methanol (1.5 mL) and brought onto an SCX-2 column (1 g) and eluted with methanol until neutral. Next, the column was eluted with ammonia in methanol (2.3 M). The basic fraction was concentrated to dryness under reduced pressure. The residue was taken up in a mixture of acetonitrile and water (1:1, 4 mL) and lyophilized to give ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (Compound 5217).

LCMS: 99%, RT=1.08 min., (M+H)⁺=399 (method P). SFC: 99%, RT=2.51 min., (M+H)⁺=399 (method AS).

### Example 91

### ((2S,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5218)

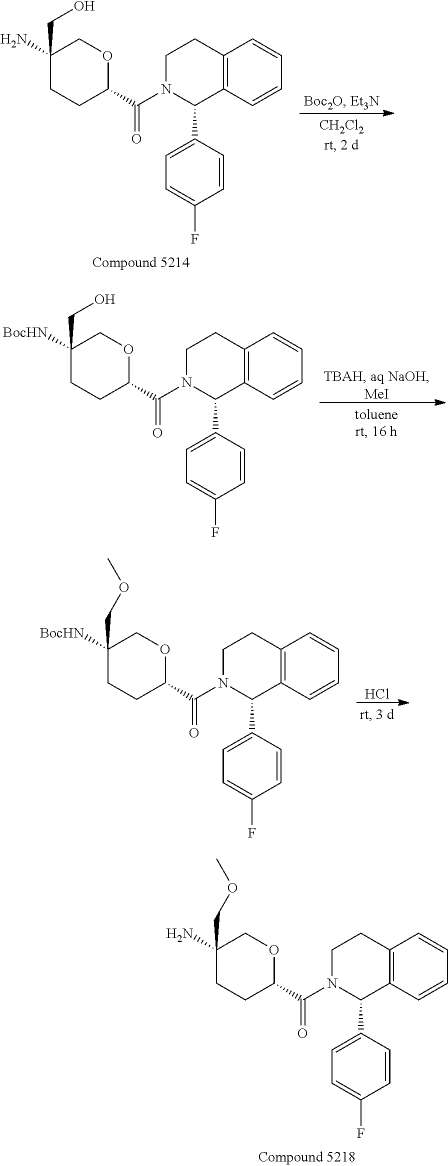

### Synthesis of tert-butyl ((3R,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate

Starting from ((2S,5R)-5-amino-5-(hydroxymethyl)tetrahydro-2H-pyran-2-yl)((S)-1-(4-fluorophenyl)-3,4-dihydroisoquinolin-2(1H)-yl)methanone (Compound 5214, 56.1 mg, 0.146 mmol), ((3R,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate (see Compound 5214).

LCMS: 94%, RT=2.08 min., (M+H)⁺=485 (method A).

### Synthesis of tert-butyl ((3R,6S)-6-((S)-1-(4-fluorophenyl)-1,2,3,4-tetrahydroisoquinoline-2-carbonyl)-3-(methoxymethyl)tetrahydro-2H-pyran-3-yl)carbamate

Starting from tert-butyl ((3R,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate (28 mg, 0.058 mmol), tert-butyl ((3R,6S)-6-((S)-1-(4-fluorophenyl)-1,2,3,4-tetrahydroisoquinoline-2-carbonyl)-3-(methoxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (see Compound 5217).

LCMS: 99%, RT=2.22 min., (M+H)⁺=499 (method A).

### Synthesis of ((2S,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5218)

Starting from tert-butyl ((3R,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (11 mg, 0.022 mmol), ((2S,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5218) was prepared as described for ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (see Compound 5217).

LCMS: 99%, RT=1.07 min., (M+H)⁺=399 (method P).

### Example 92

### ((2R,4S,5R)-5-(allyloxy)-4-aminotetrahydro-2H-pyran-2-yl)((S)-1-(4-fluorophenyl)-3,4-dihydroisoquinolin-2(1H)-yl)methanone (COMPOUND 5476)

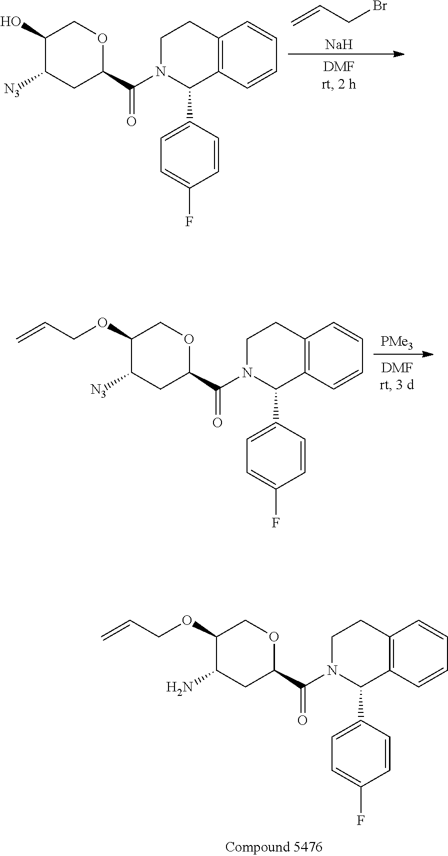

### Synthesis of ((2R,4S,5R)-5-(allyloxy)-4-azidotetrahydro-2H-pyran-2-yl)((S)-1-(4-fluorophenyl)-3,4-dihydroisoquinolin-2(1H)-yl)methanone

Under nitrogen atmosphere, sodium hydride (60% dispersion in mineral oil, 13.1 mg, 0.272 mmol) was added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 90 mg, 0.227 mmol) in N,N-dimethylformamide (dry, 4 mL). After 10 minutes, allyl bromide (0.030 mL, 0.341 mmol) was added. After 2 hours, the mixture was diluted with water and extracted with ethyl acetate. The combined organics were dried over Na₂SO₄ and evaporated under reduced pressure. The residue was purified by flash column chromatography (silica, 0 to 100% ethyl acetate in heptane) to give ((2R,4S,5R)-5-(allyloxy)-4-azidotetrahydro-2H-pyran-2-yl)((S)-1-(4-fluorophenyl)-3,4-dihydroisoquinolin-2(1H)-yl)methanone.

LCMS: 98%, RT=2.24 min., (M+H)⁺=437 (method A).

### Synthesis of ((2R,4S,5R)-5-(allyloxy)-4-aminotetrahydro-2H-pyran-2-yl)((S)-1-(4-fluorophenyl)-3,4-dihydroisoquinolin-2(1H)-yl)methanone (COMPOUND 5476)

Under nitrogen atmosphere, trimethylphosphine (1.0 M in tetrahydrofuran, 0.149 mL, 0.149 mmol) was added to a solution of ((2R,4S,5R)-5-(allyloxy)-4-azidotetrahydro-2H-pyran-2-yl)((S)-1-(4-fluorophenyl)-3,4-dihydroisoquinolin-2(1H)-yl)methanone (65 mg, 0.149 mmol) in tetrahydrofuran (dry, 4 mL). After stirring over the weekend, another portion of trimethylphosphine (1.0 M in tetrahydrofuran, 0.179 mL, 0.179 mmol) was added and stirring was continued for another 4 hours. Then, aqueous NaOH (1 M, 3 mL) was added. The mixture was stirred vigorously and was extracted with dichloromethane. The layers were separated using a phase-separator and the organic filtrate was evaporated under reduced pressure. The residue was dissolved in methanol and brought onto an SCX-2 column (2 g) and eluted with methanol until neutral. Next, the column was eluted with ammonia in methanol (2 M). The basic fraction was concentrated to dryness under reduced pressure to give ((2R,4S,5R)-5-(allyloxy)-4-aminotetrahydro-2H-pyran-2-yl)((S)-1-(4-fluorophenyl)-3,4-dihydroisoquinolin-2(1H)-yl)methanone after lyophilization from a mixture of acetonitrile and water (1:1, 4 mL).

LCMS: 95%, RT=1.17 min., (M+H)⁺=411 (method P).

### Example 93

### ((2R,4S,5S)-4-amino-5-(difluoromethoxy)tetrahydro-2H-pyran-2-yl)((S)-1-(4-fluorophenyl)-3,4-dihydroisoquinolin-2(1H)-yl)methanone (COMPOUND 5443)

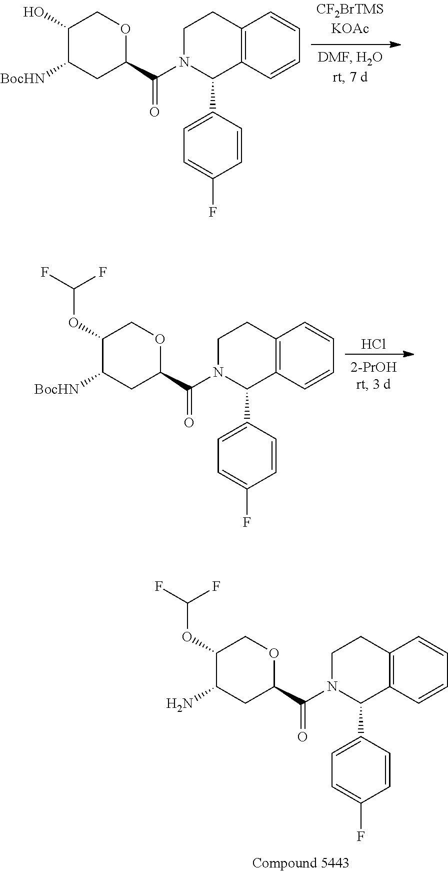 [SMILES: CC(C)(C)OC(=O)N[C@H]1C[C@H](C(=O)N2CCc3ccccc3[C@@H]2c2ccc(F)cc2)OC[C@H]1O ; CC(C)(C)OC(=O)N[C@H]1C[C@H](C(=O)N2CCc3ccccc3[C@@H]2c2ccc(F)cc2)OC[C@H]1OC(F)F ; Cl ; N[C@H]1C[C@H](C(=O)N2CCc3ccccc3[C@@H]2c2ccc(F)cc2)OC[C@H]1OC(F)F]

### Synthesis of tert-butyl ((2R,4S,5S)-5-(difluoromethoxy)-2-((S)-1-(4-fluorophenyl)-1,2,3,4-tetrahydroisoquinoline-2-carbonyl)tetrahydro-2H-pyran-4-yl)carbamate

In 5 minutes, trimethyl(bromodifluoromethyl)silane (0.066 mL, 0.425 mmol) was added portion wise to a mixture of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 100 mg, 0.213 mmol) and potassium acetate (83 mg, 0.850 mmol) in a mixture of dichloromethane (0.2 mL) and water (0.2 mL). Additional amounts of potassium acetate (83 mg, 0.850 mmol) and trimethyl(bromodifluoromethyl)silane (0.066 mL, 0.425 mmol) were added each day for 4 days in a row. Stirring was continued for another 3 days and then the mixture was diluted with dichloromethane (8 mL) and water (8 mL). The layers were separated using a phase-separator and the organic filtrate was evaporated under reduced pressure. The residue was purified by flash chromatography (silica, 0 to 40% ethyl acetate in heptane) to give tert-butyl ((2R,4S,5S)-5-(difluoromethoxy)-2-((S)-1-(4-fluorophenyl)-1,2,3,4-tetrahydroisoquinoline-2-carbonyl)tetrahydro-2H-pyran-4-yl)carbamate (33.8 mg) as a white solid.

LCMS: 81%, RT=2.21 min., (M+H)⁺=521 (method A).

### Synthesis of ((2R,4S,5S)-4-amino-5-(difluoromethoxy)tetrahydro-2H-pyran-2-yl)((S)-1-(4-fluorophenyl)-3,4-dihydroisoquinolin-2(1H)-yl)methanone (COMPOUND 5443)

HCl in 2-propanol (5.5 M, 0.452 mL, 2.486 mmol) was added to a solution of tert-butyl ((2R,4S,5S)-5-(difluoromethoxy)-2-((S)-1-(4-fluorophenyl)-1,2,3,4-tetrahydroisoquinoline-2-carbonyl)tetrahydro-2H-pyran-4-yl)carbamate (43 mg, 0.083 mmol) in 2-propanol (4 mL). After stirring for 3 days, the reaction mixture was diluted with dichloromethane (20 mL) and neutralized with saturated aqueous NaHCO₃. The layers were separated by a phase-separator and the organic filtrate was evaporated under reduced pressure. The residue was dissolved in methanol (1 mL) and brought on an SCX-2 column (1 g) and eluted with methanol until neutral. Next, the column was eluted with ammonia in methanol (7 M). The basic fraction was concentrated to dryness under reduced pressure. The residue was lyophilized from a mixture of acetonitrile and water (1:1, 4 mL) to give ((2R,4S,5S)-4-amino-5-(difluoromethoxy)tetrahydro-2H-pyran-2-yl)((S)-1-(4-fluorophenyl)-3,4-dihydroisoquinolin-2(1H)-yl)methanone (COMPOUND 5443).

LCMS: 95%, RT=1.112 min., (M+H)⁺=421 (method P).

### Example 94

### ((2R,4S,5S)-4-(ethylamino)-5-hydroxytetrahydro-2H-pyran-2-yl)((S)-1-(4-fluorophenyl)-3,4-dihydroisoquinolin-2(1H)-yl)methanone (COMPOUND 5508)

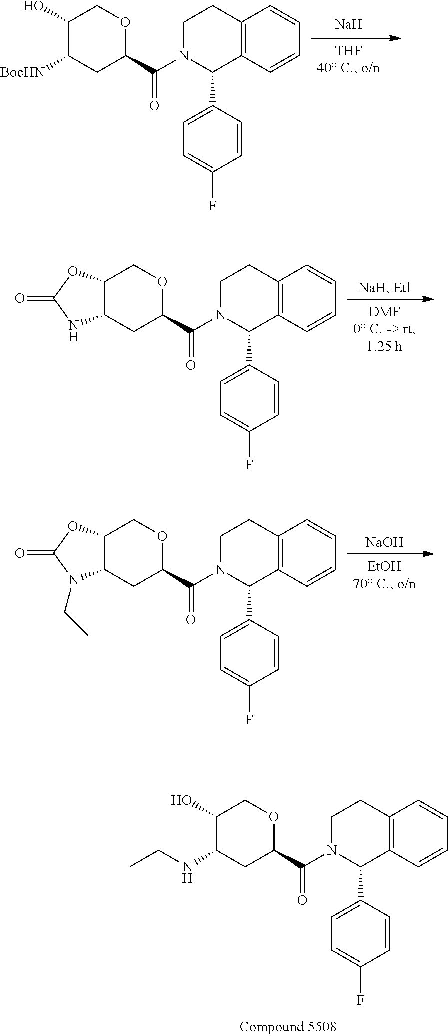

### Synthesis of (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one

Under an argon atmosphere, sodium hydride (60 wt % in mineral oil, 8.50 mg, 0.213 mmol) was added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 100 mg, 0.213 mmol) in tetrahydrofuran (dry, 5 mL). The reaction mixture was warmed to 40° C. and stirred overnight. Then, the mixture was diluted with dichloromethane (15 mL) and saturated aqueous NH₄Cl (10 mL). The layers were separated using a phase-separator and the organic filtrate was evaporated under reduced pressure. The residue was purified by flash chromatography (silica, 30 to 100% ethyl acetate in heptane) to give (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one.

LCMS: 99%, RT=1.87 min, (M+H)⁺=397 (method A).

### Synthesis of (3aS,6R,7aS)-1-ethyl-6-((S)-1-(4-fluorophenyl)-1,2,3,4-tetrahydroisoquinoline-2-carbonyl)hexahydro-2H-pyrano[4,3-d]oxazol-2-one

Under argon atmosphere at 0° C., sodium hydride (60 wt % in mineral oil, 6.93 mg, 0.173 mmol) was added to a solution of (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one (57.2 mg, 0.144 mmol) in N,N-dimethylformamide (dry, 1.5 mL). After 15 minutes, ethyl iodide (0.058 mL, 0.721 mmol) was added. After 1 hour, the reaction mixture was quenched with water (4 mL) and diluted with ethyl acetate. The layers were separated and the aqueous phase was extracted with ethyl acetate (2×5 mL). The combined organics were washed with brine (5 mL), dried over Na₂SO₄, and evaporated under reduced pressure. The residue was purified by flash chromatography (silica, 30 to 100 ethyl acetate in heptane) to give (3aS,6R,7aS)-1-ethyl-6-((S)-1-(4-fluorophenyl)-1,2,3,4-tetrahydroisoquinoline-2-carbonyl)hexahydro-2H-pyrano[4,3-d]oxazol-2-one.

LCMS: 98%, RT=1.98 min, (M+H)⁺=425 (method A).

### Synthesis of ((2R,4S,5S)-4-(ethylamino)-5-hydroxytetrahydro-2H-pyran-2-yl)((S)-1-(4-fluorophenyl)-3,4-dihydroisoquinolin-2(1H)-yl)methanone (COMPOUND 5508)

Aqueous NaOH (2 M, 0.568 mL, 1.136 mmol) was added to a solution of (3aS,6R,7aS)-1-ethyl-6-((S)-1-(4-fluorophenyl)-1,2,3,4-tetrahydroisoquinoline-2-carbonyl)hexahydro-2H-pyrano[4,3-d]oxazol-2-one (48.2 mg, 0.114 mmol) in ethanol (3 mL) and the mixture was warmed to 70° C. After stirring overnight, the mixture was concentrated to dryness under reduced pressure. The residue was diluted with dichloromethane (5 mL) and saturated aqueous NaHCO₃ (5 mL). The layers were separated using a phase-separator and the organic filtrate was evaporated under reduced pressure. The residue was purified by preparative SFC (method BS) to give ((2R,4S,5S)-4-(ethylamino)-5-hydroxytetrahydro-2H-pyran-2-yl)((S)-1-(4-fluorophenyl)-3,4-dihydroisoquinolin-2(1H)-yl)methanone after lyophilization from a mixture of acetonitrile and water (1:1, 4 mL).

LCMS: 99%, RT=1.05 min., (M+H)⁺=399, (method P). SFC: 99%, RT=2.78 min., (M+H)⁺=399, (method AS).

### Example 95

### ((2R,4S,5R)-4-amino-5-(trifluoromethoxy)tetrahydro-2H-pyran-2-yl)((S)-1-(4-fluorophenyl)-3,4-dihydroisoquinolin-2(1H)-yl)methanone (COMPOUND 5507)

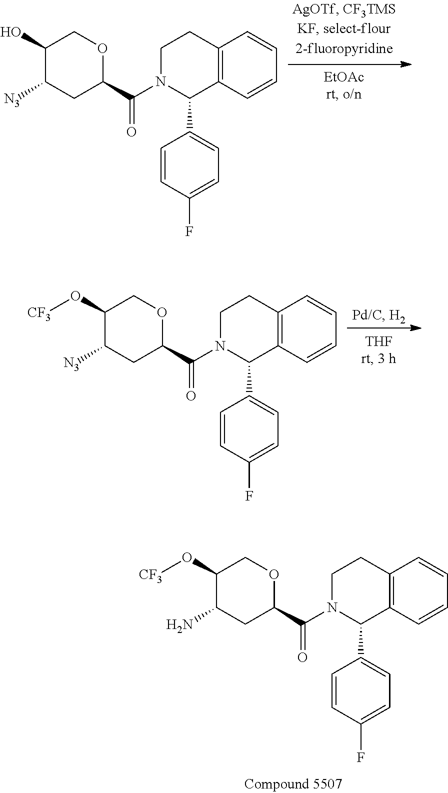

### Synthesis of ((2R,4S,5R)-4-azido-5-(trifluoromethoxy)tetrahydro-2H-pyran-2-yl)((S)-1-(4-fluorophenyl)-3,4-dihydroisoquinolin-2(1H)-yl)methanone

((2R,4S,5R)-4-Azido-5-hydroxytetrahydro-2H-pyran-2-yl)((S)-1-(4-fluorophenyl)-3,4-dihydroisoquinolin-2(1H)-yl)methanone (see Compound 5075, 313 mg, 0.790 mmol) was concentrated from ethyl acetate (10 mL), placed under argon, and transferred into a glove box. Selectfluor (420 mg, 1.184 mmol), silver triflate (609 mg, 2.369 mmol), and potassium fluoride (anhydrous, 138 mg, 2.369 mmol) were added. The reaction flask was covered in aluminium foil. Then, consecutively, ethyl acetate (dry, 4 mL), 2-fluoropyridine (0.272 mL, 3.16 mmol), and (trifluoromethyl)trimethylsilane (0.376 mL, 2.369 mmol) were added and the mixture was stirred overnight.

Then, the mixture was diluted with ethyl acetate (10 mL), filtered through a layer of celite, and rinsed with ethyl acetate (3×10 mL). The combined filtrates were diluted with saturated aqueous NaHCO₃ (40 mL) and the resulting mixture was filtered through celite. The residue was rinsed with ethyl acetate (3×5 mL). The layers of the combined filtrates were separated and the organic layer was washed with brine (30 mL), dried on Na₂SO₄, and concentrated under reduced pressure. The residue was dissolved in toluene, coated on hydromatrix, and purified by flash column chromatography (silica, 5 to 60% ethyl acetate in heptane) to give ((2R,4S,5R)-4-azido-5-(trifluoromethoxy)tetrahydro-2H-pyran-2-yl)((S)-1-(4-fluorophenyl)-3,4-dihydroisoquinolin-2(1H)-yl)methanone.

LCMS: 95%, RT=2.26 min., (M+H)⁺=465 (method A).

### Synthesis of ((2R,4S,5R)-4-amino-5-(trifluoromethoxy)tetrahydro-2H-pyran-2-yl)((S)-1-(4-fluorophenyl)-3,4-dihydroisoquinolin-2(1H)-yl)methanone (COMPOUND 5507)

A solution of ((2R,4S,5R)-4-azido-5-(trifluoromethoxy)tetrahydro-2H-pyran-2-yl)((S)-1-(4-fluorophenyl)-3,4-dihydroisoquinolin-2(1H)-yl)methanone (154 mg, 0.332 mmol) in tetrahydrofuran (5 mL) was hydrogenated in the presence of palladium (10 wt % on carbon, containing 50% water, 50 mg, 0.023 mmol) atmospheric hydrogen pressure. After 3 hours, the reaction mixture was diluted with methanol (10 mL), filtered, and evaporated under reduced pressure. The residue was purified by acidic preparative MPLC (Linear Gradient: t=0 min 10% A; t=2 min 10% A; t=17 min 50% A; t=18 min 100%; t=23 min 100% A; detection: 215/254 nm). The product fractions were combined, diluted with saturated aqueous NaHCO₃, partially concentrated to remove the acetonitrile, and extracted with dichloromethane. The organic layer was passed through a phase-separator and the organic filtrate was evaporated under reduced pressure. The residue was taken up in a mixture of acetonitrile and water (1:1, 15 mL), filtered through a nylon (0.45 [symbol] m) filter, and lyophilized to give ((2R,4S,5R)-4-amino-5-(trifluoromethoxy)tetrahydro-2H-pyran-2-yl)((S)-1-(4-fluorophenyl)-3,4-dihydroisoquinolin-2(1H)-yl)methanone (COMPOUND 5507).

LCMS: 99%, RT=2.95 min., (M+H)⁺=439 (method AK).

### Example 96

### ((2R,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5219)

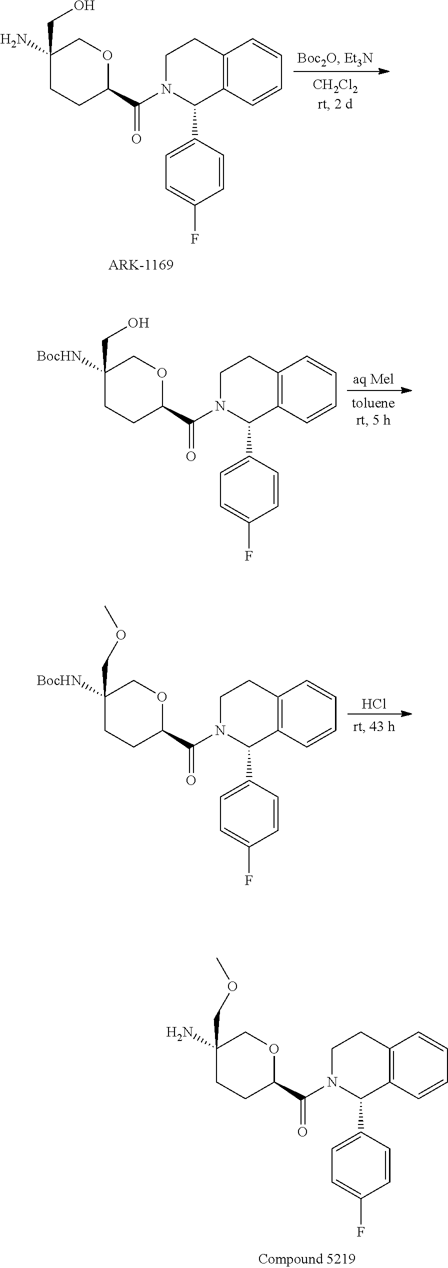

### Synthesis of tert-butyl ((3R,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate

Starting from crude ((2R,5R)-5-amino-5-(hydroxymethyl)tetrahydro-2H-pyran-2-yl)((S)-1-(4-fluorophenyl)-3,4-dihydroisoquinolin-2(1H)-yl)methanone (see Compound 5215, 0.145 mmol), tert-butyl ((3R,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate (see Compound 5217). The stereochemistry of the 5-amino-pyran center is arbitrarily assigned.

LCMS: 98%, RT=2.09 min (M+H)⁺=485 (method A).

### Synthesis of tert-butyl ((3R,6R)-6-((S)-1-(4-fluorophenyl)-1,2,3,4-tetrahydroisoquinoline-2-carbonyl)-3-(methoxymethyl)tetrahydro-2H-pyran-3-yl)carbamate

Starting from tert-butyl ((3R,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate (34 mg, 0.070 mmol), tert-butyl ((3R,6R)-6-((S)-1-(4-fluorophenyl)-1,2,3,4-tetrahydroisoquinoline-2-carbonyl)-3-(methoxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (see Compound 5217). The stereochemistry of the 5-amino-pyran center is arbitrarily assigned.

LCMS: 99%, RT=2.23 min., (M+H)⁺=499 (method A).

### Synthesis of ((2R,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5219)

Starting from tert-butyl ((3R,6R)-6-((S)-1-(4-fluorophenyl)-1,2,3,4-tetrahydroisoquinoline-2-carbonyl)-3-(methoxymethyl)tetrahydro-2H-pyran-3-yl)carbamate (13 mg, 0.026 mmol), ((2R,5R)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5219) was prepared as described for ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (see Compound 5217). The stereochemistry of the 5-amino-pyran center is arbitrarily assigned.

LCMS: 99%, RT=1.08 min., (M+H)⁺=399 (method P).

### Example 97

### ((2R,5S)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5220)

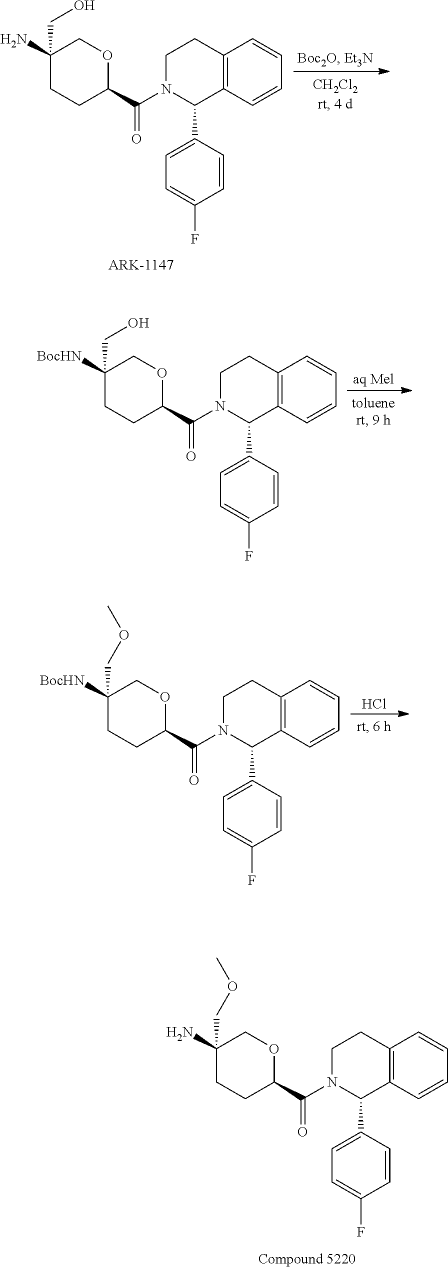

### Synthesis of tert-butyl ((3S,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate

Starting from crude ((2R,5S)-5-amino-5-(hydroxymethyl)tetrahydro-2H-pyran-2-yl)((S)-1-(4-fluorophenyl)-3,4-dihydroisoquinolin-2(1H)-yl)methanone (see Compound 5216, 0.145 mmol), tert-butyl ((3S,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(hydroxymethyl)tetrahydro-2H-pyran-3-yl)carbamate (see Compound 5217). The stereochemistry of the 5-amino-pyran center is arbitrarily assigned.

LCMS: 99%, RT=2.06 min (M+H)⁺=485 (method A).

### Synthesis of tert-butyl ((3S,6R)-6-((S)-1-(4-fluorophenyl)-1,2,3,4-tetrahydroisoquinoline-2-carbonyl)-3-(methoxymethyl)tetrahydro-2H-pyran-3-yl)carbamate

Starting from tert-butyl ((3S,6R)-6-((S)-1-(4-fluorophenyl)-1,2,3,4-tetrahydroisoquinoline-2-carbonyl)-3-(hydroxymethyl)tetrahydro-2H-pyran-3-yl)carbamate (17 mg, 0.035 mmol), tert-butyl ((3S,6R)-6-((S)-1-(4-fluorophenyl)-1,2,3,4-tetrahydroisoquinoline-2-carbonyl)-3-(methoxymethyl)tetrahydro-2H-pyran-3-yl)carbamate was prepared as described for tert-butyl ((3S,6S)-6-((S)-1-(4-fluorophenyl)-1,2,3,4-tetrahydroisoquinoline-2-carbonyl)-3-(methoxymethyl)tetrahydro-2H-pyran-3-yl)carbamate (see Compound 5217). The stereochemistry of the 5-amino-pyran center is arbitrarily assigned.

LCMS: 99%, RT=2.21 min., (M+H)⁺=499 (method A).

### Synthesis of ((2R,5S)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5220)

Starting from tert-butyl ((3S,6R)-6-((S)-1-(4-fluorophenyl)-1,2,3,4-tetrahydroisoquinoline-2-carbonyl)-3-(methoxymethyl)tetrahydro-2H-pyran-3-yl)carbamate (6 mg, 0.012 mmol), ((2R,5S)-5-amino-5-(methoxymethyl)tetrahydro-2H-pyran-2-yl)((S)-1-(4-fluorophenyl)-3,4-dihydroisoquinolin-2(1H)-yl)methanone (COMPOUND 5220) was prepared as described for ((2S,5S)-5-amino-5-(methoxymethyl)tetrahydro-2H-pyran-2-yl)((S)-1-(4-fluorophenyl)-3,4-dihydroisoquinolin-2(1H)-yl)methanone (see Compound 5217). The stereochemistry of the 5-amino-pyran center is arbitrarily assigned.

LCMS: 94%, RT=1.08 min., (M+H)⁺=399 (method P, 40° C.).

### Example 98

### ((2R,4S,5S)-4-amino-5-(methylthio)tetrahydro-2H-pyran-2-yl)((S)-1-(4-fluorophenyl)-3,4-dihydroisoquinolin-2(1H)-yl)methanone (COMPOUND 5425)

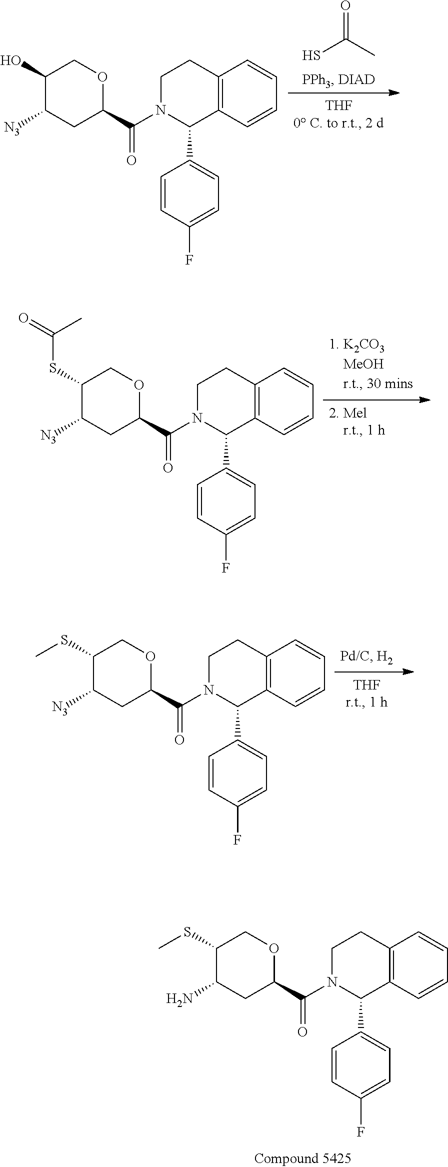

### Synthesis of S-((3S,4S,6R)-4-azido-6-((S)-1-(4-fluorophenyl)-1,2,3,4-tetrahydroisoquinoline-2-carbonyl)tetrahydro-2H-pyran-3-yl) ethanethioate

Under an argon atmosphere at 0° C., diisopropyl azodicarboxylate (0.221 mL, 1.135 mmol) was added to a solution of triphenylphosphine (298 mg, 1.14 mmol) in tetrahydrofuran (5 mL). The mixture was warmed to 30° C., stirred for 1 hour, and cooled to 0° C. again. A solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 300 mg, 0.757 mmol) and thioacetic acid (0.082 mL, 1.135 mmol) in tetrahydrofuran (5 mL) was added and after 1 hour, the mixture was allowed to warm to room temperature and stirred overnight. In a separate flask under an argon atmosphere at 0° C., diisopropyl azodicarboxylate (0.221 mL, 1.135 mmol) was added to a solution of triphenylphosphine (298 mg, 1.135 mmol) in tetrahydrofuran (5 mL) and stirred for 1 hour at 0° C. Then, the first reaction mixture was added followed by the addition of thioacetic acid (0.054 mL, 0.757 mmol). This mixture was allowed to warm up to room temperature and stirred for one more day. The mixture was concentrated under reduced pressure and the residue was purified by flash column chromatography (silica, 5 to 35% ethyl acetate in heptane) to give S-((3S,4S,6R)-4-azido-6-((S)-1-(4-fluorophenyl)-1,2,3,4-tetrahydroisoquinoline-2-carbonyl)tetrahydro-2H-pyran-3-yl) ethanethioate.

LCMS: 79%, RT=2.25 min., (M+H)⁺=455 (method B).

### Synthesis of ((2R,4S,5S)-4-azido-5-(methylthio)tetrahydro-2H-pyran-2-yl)((S)-1-(4-fluorophenyl)-3,4-dihydroisoquinolin-2(1H)-yl)methanone

Potassium carbonate (85 mg, 0.616 mmol) was added to a solution of S-((3S,4S,6R)-4-azido-6-((S)-1-(4-fluorophenyl)-1,2,3,4-tetrahydroisoquinoline-2-carbonyl)tetrahydro-2H-pyran-3-yl) ethanethioate (106 mg, 0.205 mmol) in methanol (4 mL). The resulting suspension was stirred at room temperature for 30 minutes. Methyl iodide (0.030 mL, 0.480 mmol) was added and stirring was continued for 1 hour. The mixture was concentrated under reduced pressure and the residue was diluted with dichloromethane (3 mL) and water (3 mL). The organic phase was passed through a phase separator and the organic filtrate was concentrated under reduced pressure. The residue was purified by flash column chromatography (silica, 5 to 30% ethyl acetate in heptane) to give ((2R,4S,5S)-4-azido-5-(methylthio)tetrahydro-2H-pyran-2-yl)((S)-1-(4-fluorophenyl)-3,4-dihydroisoquinolin-2(1H)-yl)methanone.

LCMS: 100%, RT=2.20 min., (M+H)⁺=427 (method A).

### Synthesis of ((2R,4S,5S)-4-amino-5-(methylthio)tetrahydro-2H-pyran-2-yl)((S)-1-(4-fluorophenyl)-3,4-dihydroisoquinolin-2(1H)-yl)methanone (COMPOUND 5425)

A solution of ((2R,4S,5S)-4-azido-5-(methylthio)tetrahydro-2H-pyran-2-yl)((S)-1-(4-fluorophenyl)-3,4-dihydroisoquinolin-2(1H)-yl)methanone (30 mg, 0.070 mmol) in tetrahydrofuran (1 mL) was hydrogenated in the presence of palladium (10 wt % on carbon, containing 50% water, 60 mg, 0.045 mmol) at atmospheric hydrogen pressure. After 1 hour, the mixture was filtered over a 45 μm nylon filter and the filtrate was concentrated under reduced pressure. The residue was purified by acidic preparative MPLC (Linear Gradient: t=0 min 5% A; t=1 min 10% A; t=16 min 50% A; t=17 min 100%; t=22 min 100% A; detection: 220/264 nm). The product fractions were combined and concentrated under reduced pressure. The residue was dissolved in methanol (2 mL) and brought onto an SCX-2 column (1 g) and eluted with methanol until neutral. Next, the column was eluted with ammonia in methanol (2 M). The basic fraction was concentrated to dryness under reduced pressure. The residue was lyophilised from a mixture of acetonitrile and water (1:1, 2 mL) to give ((2R,4S,5S)-4-amino-5-(methylthio)tetrahydro-2H-pyran-2-yl)((S)-1-(4-fluorophenyl)-3,4-dihydroisoquinolin-2(1H)-yl)methanone (COMPOUND 5425).

LCMS: 100%, RT=1.11 min., (M+H)⁺=401 (method P).

### Example 99

### ((2R,4S,5S)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5426)

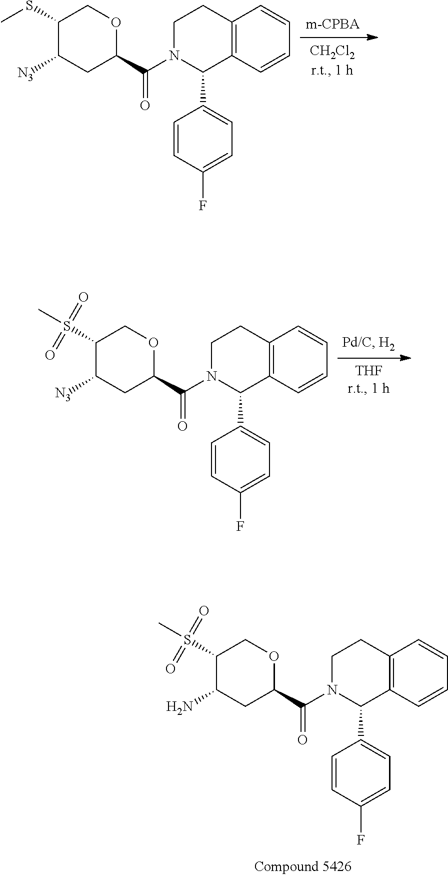 [SMILES: CS[C@@H]1CO[C@@H](C(=O)N2CCc3ccccc3[C@@H]2c2ccc(F)cc2)C[C@@H]1N=[N+]=[N-] ; CS(=O)(=O)[C@@H]1CO[C@@H](C(=O)N2CCc3ccccc3[C@@H]2c2ccc(F)cc2)C[C@@H]1N=[N+]=[N-] ; CS(=O)(=O)[C@@H]1CO[C@@H](C(=O)N2CCc3ccccc3[C@@H]2c2ccc(F)cc2)C[C@@H]1N]

### Synthesis of ((2R,4S,5S)-4-azido-5-(methylsulfonyl)tetrahydro-2H-pyran-2-yl)((S)-1-(4-fluorophenyl)-3,4-dihydroisoquinolin-2(1H)-yl)methanone

meta-Chloroperoxybenzoic acid (70 wt %, 49.6 mg, 0.201 mmol) was added to a solution of ((2R,4S,5S)-4-azido-5-(methylthio)tetrahydro-2H-pyran-2-yl)((S)-1-(4-fluorophenyl)-3,4-dihydroisoquinolin-2(1H)-yl)methanone (see Compound 5425, 39 mg, 0.091 mmol) in dichloromethane (2 mL). The mixture was stirred at room temperature for 1 hour. The mixture was diluted with dichloromethane (5 mL) and a mixture of saturated aqueous Na₂S₂O₃, saturated aqueous NaHCO₃, and water (1:1:1, 10 mL). The mixture was stirred at room temperature for 10 minutes. The layers were separated over a phase separation filter. The organic filtrate was concentrated under reduced pressure to obtain ((2R,4S,5S)-4-azido-5-(methylsulfonyl)tetrahydro-2H-pyran-2-yl)((S)-1-(4-fluorophenyl)-3,4-dihydroisoquinolin-2(1/H)-yl)methanone.

LCMS: 96%, RT=2.05 min., (M+H)⁺=459 (method A).

### Synthesis of ((2R,4S,5S)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5426)

A solution of ((2R,4S,5S)-4-azido-5-(methylsulfonyl)tetrahydro-2H-pyran-2-yl)((S)-1-(4-fluorophenyl)-3,4-dihydroisoquinolin-2(1H)-yl)methanone (40 mg, 0.087 mmol) in tetrahydrofuran (3 mL) was hydrogenated in the presence of palladium (10 wt % on carbon, containing 50% water, spatula tip) at atmospheric hydrogen pressure. After 1 hour, the mixture was filtered over a 45 μm nylon filter and the filtrate was concentrated under reduced pressure. The residue was dissolved in methanol (2 mL) and brought onto an SCX-2 column (1 g) and eluted with methanol until neutral. Next, the column was eluted with ammonia in methanol (2 M). The basic fraction was concentrated to dryness under reduced pressure. The residue was lyophilised from a mixture of acetonitrile and water (1:1, 2 mL) to give ((2R,4S,5S)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5426).

LCMS: 99%, RT=2.86 min., (M+H)⁺=433 (method AK).

### Example 100

### ((S)-1-(4-fluorophenyl)-3,4-dihydroisoquinolin-2(1H)-yl)((4aS,7R,8aS)-octahydropyrano[3,4-b][1,4]oxazin-7-yl)methanone (COMPOUND 5448)

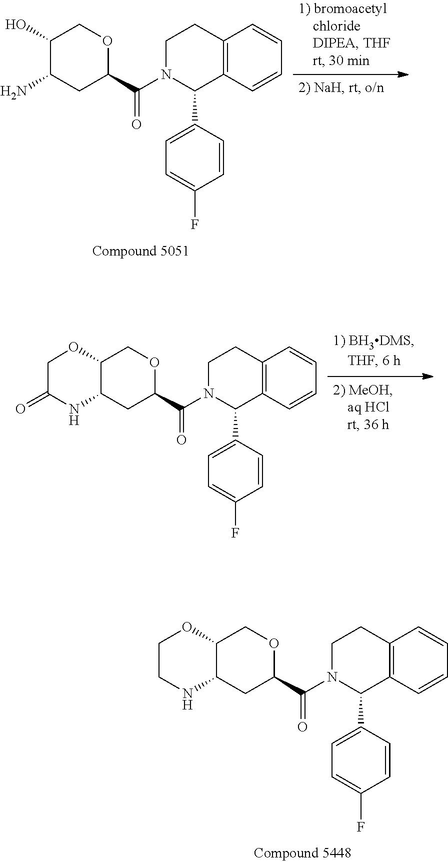 [SMILES: N[C@H]1C[C@H](C(=O)N2CCc3ccccc3[C@@H]2c2ccc(F)cc2)OC[C@H]1O ; O=C1CO[C@@H]2CO[C@@H](C(=O)N3CCc4ccccc4[C@@H]3c3ccc(F)cc3)C[C@@H]2N1 ; O=C([C@H]1C[C@@H]2NCCO[C@@H]2CO1)N1CCc2ccccc2[C@@H]1c1ccc(F)cc1]

### Synthesis of (4aS,7R,8aS)-7-((S)-1-(4-fluorophenyl)-1,2,3,4-tetrahydroisoquinoline-2-carbonyl)hexahydropyrano[3,4-b][1,4]oxazin-2(3H)-one

A solution of bromoacetyl chloride (0.050 mL, 0.594 mmol) in tetrahydrofuran (5 mL) was added to a solution of ((2R,4S,5S)-4-amino-5-hydroxytetrahydro-2H-pyran-2-yl)((S)-1-(4-fluorophenyl)-3,4-dihydroisoquinolin-2(1H)-yl)methanone (200 mg, 0.540 mmol) and N,N-diisopropylethylamine (0.283 mL, 1.620 mmol) in tetrahydrofuran (5 mL). After 30 minutes, sodium hydride (60 wt % dispersion in mineral oil, 64.8 mg, 1.620 mmol) was added and the mixture was stirred overnight. During the day, additional portions of sodium hydride (60 wt % dispersion in mineral oil, 13 mg, 0.325 mmol and 6.5 mg, 0.163 mmol) were added and stirring was continued overnight. Molecular sieves and more sodium hydride (60 wt % dispersion in mineral oil, 13 mg, 0.325 mmol) were added and stirring was continued for one more day. The reaction mixture was diluted with saturated aqueous ammonium chloride (15 mL) and dichloromethane (30 mL). The organic layer was collected, washed with brine (20 mL), dried over Na₂SO₄ and evaporated under reduced pressure. The residue was purified by flash column chromatography (silica, 0 to 100% ethyl acetate in heptane) to give (4aS,7R,8aS)-7-((S)-1-(4-fluorophenyl)-1,2,3,4-tetrahydroisoquinoline-2-carbonyl)hexahydropyrano[3,4-b][1,4]oxazin-2(3H)-one.

LCMS: 98%, RT=1.89 min., (M+H)⁺=411 (method A).

### Synthesis of ((S)-1-(4-fluorophenyl)-3,4-dihydroisoquinolin-2(1H)-yl)((4aS,7R,8aS)-octahydropyrano[3,4-b][1,4]oxazin-7-yl)methanone (COMPOUND 5448)

Borane-dimethyl sulfide complex (2 M in tetrahydrofuran, 0.453 mL, 0.906 mmol) was added to a solution of ((4aS,7R,8aS)-7-((S)-1-(4-fluorophenyl)-1,2,3,4-tetrahydroisoquinoline-2-carbonyl)hexahydropyrano[3,4-b][1,4]oxazin-2(3H)-one (93 mg, 0.227 mmol) in tetrahydrofuran (dry, 1 mL). After 6 hours, the reaction mixture was diluted with a mixture of methanol and aqueous HCl (1 M) (1:1, 6 mL). After 36 hours, the reaction mixture was diluted with dichloromethane (15 mL) and saturated aqueous NaHCO₃ (15 mL). The layers were separated using a phase-separator and the organic filtrate was evaporated under reduced pressure. The residue was combined with the crude product of the same reaction (starting from 31 mg, 0.076 mmol), dissolved in methanol (2 mL), brought onto an SCX-column (2 g), and eluted with methanol until neutral. Next, the column was eluted with ammonia in methanol (1.5 M). The basic fraction was concentrated to dryness under reduced pressure. The residue was purified by preparative SFC (method AW) to give ((S)-1-(4-fluorophenyl)-3,4-dihydroisoquinolin-2(1H)-yl)((4aS,7R,8aS)-octahydropyrano[3,4-b][1,4]oxazin-7-yl)methanone (COMPOUND 5448) after lyophilization from a mixture of acetonitrile and water (1:1, 3 mL).

LCMS: 99%, RT=1.06 min., (M+H)⁺=397 (method P). SFC: 98%, RT=3.55 min., (M+H)⁺=397 (method W).

### Example 101

### ((2R,4S,5R)-4-amino-5-isopropoxytetrahydro-2H-pyran-2-yl)((S)-1-(4-fluorophenyl)-3,4-dihydroisoquinolin-2(1H)-yl)methanone (COMPOUND 5267)

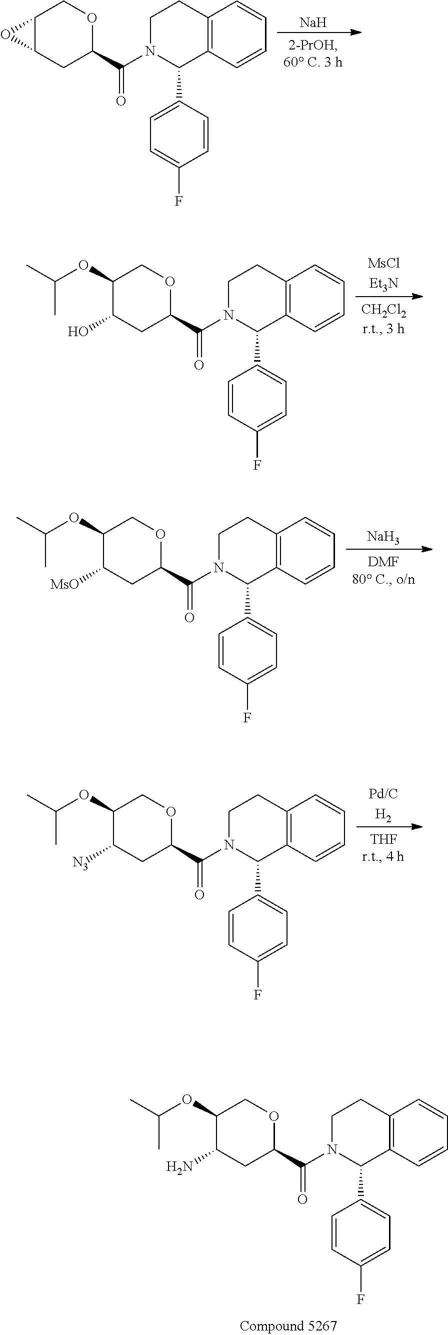

### Synthesis of ((S)-1-(4-fluorophenyl)-3,4-dihydroisoquinolin-2(1H)-yl)((2R,4S,5S)-4-hydroxy-5-isopropoxytetrahydro-2H-pyran-2-yl)methanone

Under an argon atmosphere, a hot solution of ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 300 mg, 0.849 mmol) in 2-propanol (1.5 mL) was added to a suspension of sodium hydride (60 wt % in mineral oil, 67.9 mg, 1.698 mmol) in 2-propanol (3 mL). After stirring at 60° C. for 3 hours, the reaction mixture was poured into saturated aqueous NH₄Cl (10 mL) and the resulting mixture was extracted with ethyl acetate (20 mL). The layers were separated and the organic phase was washed with brine (10 mL), dried on Na₂SO₄, and concentrated under reduced pressure. The residue was purified by flash column chromatography (silica, 20 to 70% ethyl acetate in heptane) to give ((S)-1-(4-fluorophenyl)-3,4-dihydroisoquinolin-2(1H)-yl)((2R,4S,5S)-4-hydroxy-5-isopropoxytetrahydro-2H-pyran-2-yl)methanone.

LCMS: 96%, RT=2.01 min., (M+H)⁺=414 (method A). SFC: 96%, RT=2.14 min., (M+H)⁺=414 (method V).

### Synthesis of (2R,4S,5S)-2-((S)-1-(4-fluorophenyl)-1,2,3,4-tetrahydroisoquinoline-2-carbonyl)-5-isopropoxytetrahydro-2H-pyran-4-yl methanesulfonate

Triethylamine (0.145 mL, 1.040 mmol) followed by methanesulfonyl chloride (0.061 mL, 0.780 mmol) were added to a solution of ((S)-1-(4-fluorophenyl)-3,4-dihydroisoquinolin-2(1H)-yl)((2R,4S,5S)-4-hydroxy-5-isopropoxytetrahydro-2H-pyran-2-yl)methanone (215 mg, 0.520 mmol) in dichloromethane (3 mL). After 2 hours, additional triethyl amine (5 drops) and methanesulfonyl chloride (2 drops) was added and stirring was continued for another hour. Then, the mixture was diluted with saturated aqueous NaHCO₃ and stirred for 0.5 hour. The layers were separated using a phase separator and the organic filtrate was concentrated under reduced pressure to give (2R,4S,5S)-2-((S)-1-(4-fluorophenyl)-1,2,3,4-tetrahydroisoquinoline-2-carbonyl)-5-isopropoxytetrahydro-2H-pyran-4-yl methanesulfonate.

LCMS: 98%, RT=2.09 min., (M+H)⁺=492 (method B).

### Synthesis of ((2R,4S,5R)-4-azido-5-isopropoxytetrahydro-2H-pyran-2-yl)((S)-1-(4-fluorophenyl)-3,4-dihydroisoquinolin-2(1H)-yl)methanone

Sodium azide (116 mg, 1.780 mmol) was added to a solution of (2R,4S,5S)-2-((S)-1-(4-fluorophenyl)-1,2,3,4-tetrahydroisoquinoline-2-carbonyl)-5-isopropoxytetrahydro-2H-pyran-4-yl methanesulfonate (250 mg, 0.509 mmol) and the mixture was stirred at 80° C., overnight. Then, the reaction mixture was diluted with ethyl acetate (10 mL), washed with a mixture of saturated aqueous NaHCO₃ and water (1:1, 10 mL), and brine (3×10 mL), dried on Na₂SO₄, and evaporated under reduced pressure. The residue was dissolved in toluene, loaded on hydromatrix, and purified by flash column chromatography (silica, 10 to 50% ethyl acetate in heptane) to give ((2R,4S,5R)-4-azido-5-isopropoxytetrahydro-2H-pyran-2-yl)((S)-1-(4-fluorophenyl)-3,4-dihydroisoquinolin-2(1H)-yl)methanone.

LCMS: 99%, RT=2.32 min., (M+H)⁺=439 (method B).

### Synthesis of ((2R,4S,5R)-4-amino-5-isopropoxytetrahydro-2H-pyran-2-yl)((S)-1-(4-fluorophenyl)-3,4-dihydroisoquinolin-2(1H)-yl)methanone (COMPOUND 5267)

A solution of ((2R,4S,5R)-4-azido-5-isopropoxytetrahydro-2H-pyran-2-yl)((S)-1-(4-fluorophenyl)-3,4-dihydroisoquinolin-2(1H)-yl)methanone (114 mg, 0.260 mmol) in tetrahydrofuran (3 mL) was hydrogenated in the presence of palladium (10 wt % on carbon, containing 50% water, 30 mg, 0.014 mmol) and atmospheric hydrogen pressure. After 4 hours, the reaction mixture was diluted with methanol (10 mL) and filtered through a nylon filter (0.45 [symbol] m). The filtrate was evaporated under reduced pressure and the residue was purified by acidic preparative MPLC (Linear Gradient: t=0 min 10% A; t=2 min 10% A; t=17 min 50% A; t=18 min 100%; t=23 min 100% A; detection: 215 nm). The product containing fractions were combined, diluted with saturated aqueous NaHCO₃ (till basic on pH paper), and partially concentrated to remove the acetonitrile. The aqueous phase was stirred with dichloromethane for 0.5 hour after which the layers were separated using a phase separator. The organic filtrate was evaporated under reduced pressure to give ((2R,4S,5R)-4-amino-5-isopropoxytetrahydro-2H-pyran-2-yl)((S)-1-(4-fluorophenyl)-3,4-dihydroisoquinolin-2(1H)-yl)methanone (COMPOUND 5267) after lyophilization from a mixture of acetonitrile and water (1:1, 4 mL).

LCMS: 99%, RT=1.18 min., (M+H)⁺=413 (method P).

### Example 102

### ((2R,4R,5S)-4-amino-5-fluorotetrahydro-2H-pyran-2-yl)((S)-1-(4-fluorophenyl)-3,4-dihydroisoquinolin-2(1H)-yl)methanone (COMPOUND 5251)

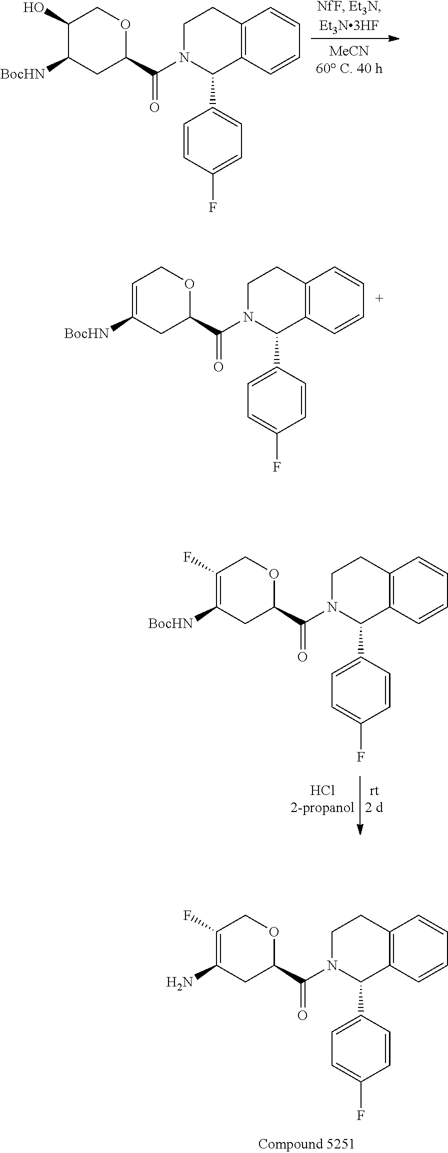

### Synthesis of tert-butyl ((2R,4R,5S)-5-fluoro-2-((S)-1-(4-fluorophenyl)-1,2,3,4-tetrahydroisoquinoline-2-carbonyl)tetrahydro-2H-pyran-4-yl)carbamate

Under a nitrogen atmosphere, triethylamine trihydrofluoride (0.208 mL, 1.275 mmol) and perfluoro-1-butanesulfonyl fluoride (0.229 mL, 1.275 mmol) were added to a solution of tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate (see Compound 5259, 200 mg, 0.425 mmol) and triethylamine (0.391 mL, 2.81 mmol) in acetonitrile (dry, 10 mL). The mixture was warmed to 60° C. and stirred overnight. Additional triethylamine (0.118 mL, 0.850 mmol), triethylamine trihydrofluoride (0.069 mL, 0.425 mmol), and perfluoro-1-butanesulfonyl fluoride (0.076 mL, 0.425 mmol) were added and stirring at 60° C. was continued for another 5 days. Then, after cooling the mixture to room temperature, it was poured onto cold saturated aqueous NaHCO₃ (10 mL) and extracted with ethyl acetate (twice). The combined organics were washed with brine, dried on Na₂SO₄, and evaporated under reduced pressure. The residue was purified by flash column chromatography (silica, 0 to 40% ethyl acetate in heptane) and preparative SFC (method AN) to give tert-butyl ((2R,4R,5S)-5-fluoro-2-((S)-1-(4-fluorophenyl)-1,2,3,4-tetrahydroisoquinoline-2-carbonyl)tetrahydro-2H-pyran-4-yl)carbamate.

LCMS: 99%, RT=2.16 min., (M+Na)⁺=495 (method B).

### Synthesis of ((2R,4R,5S)-4-amino-5-fluorotetrahydro-2H-pyran-2-yl)((S)-1-(4-fluorophenyl)-3,4-dihydroisoquinolin-2(1H)-yl)methanone (COMPOUND 5251)

HCl (5-6 M in 2-propanol, 0.5 mL, 2.75 mmol) was added to a solution of tert-butyl ((2R,4R,5S)-5-fluoro-2-((S)-1-(4-fluorophenyl)-1,2,3,4-tetrahydroisoquinoline-2-carbonyl)tetrahydro-2H-pyran-4-yl)carbamate (18 mg, 0.038 mmol) in 2-propanol (3 mL). After 2 days, the mixture was partioned between dichloromethane (25 mL) and a mixture of saturated aqueous NaHCO₃ and water (1:1, 20 mL). The layers were separated and the aqueous phase was extracted with dichloromethane (25 mL). The combined organic layers were passed through a phase separator and evaporated under reduced pressure. The residue was dissolved in methanol, brought onto an SCX-2 column (1 g) and eluted with methanol until neutral. Next, the column was eluted with ammonia in methanol (1 M). The basic fraction was concentrated to dryness under reduced pressure. The residue was lyophilised from a mixture of acetonitrile and water (1:1, 4 mL) to give ((2R,4R,5S)-4-amino-5-fluorotetrahydro-2H-pyran-2-yl)((S)-1-(4-fluorophenyl)-3,4-dihydroisoquinolin-2(1H)-yl)methanone (COMPOUND 5251).

LCMS: 99%, RT=1.06 min., (M+H)⁺=373 (method P).

### Example 103

### ((2R,4S,5R)-5-ethoxy-4-(ethylamino)tetrahydro-2H-pyran-2-yl)((S)-1-(4-fluorophenyl)-3,4-dihydroisoquinolin-2(1H)-yl)methanone (COMPOUND 5510

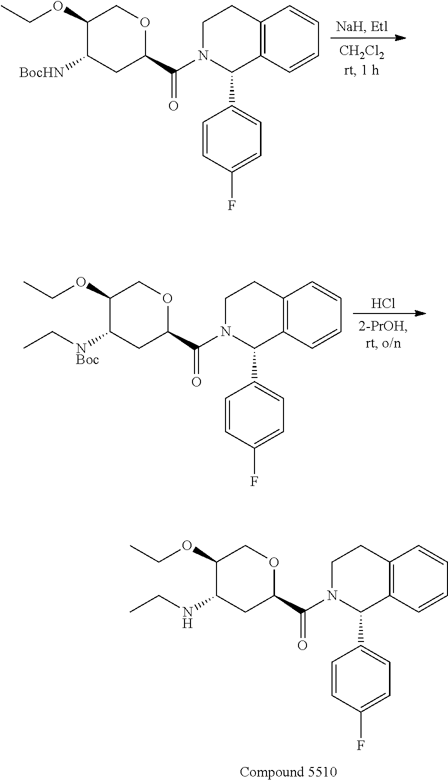

### Synthesis of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(ethyl)carbamate

Sodium hydride (60 wt % dispersion in mineral oil, 7.7 mg, 0.193 mmol) was added to a solution of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)carbamate (See Compound 5501, 80 mg, 0.160 mmol) in N,N-dimethylformamide (3 mL). After 10 minutes, iodoethane (0.038 mL, 0.481 mmol) was added. After 1 hour, the mixture was diluted with water and extracted with ethyl acetate. The organic phase was evaporated under reduced pressure. The residue was purified by flash column chromatography (silica, 20 to 100% ethyl acetate in heptane) to give tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(ethyl)carbamate.

LCMS: 99%, RT=2.38 min., (M+H)⁺=527 (method A).

### Synthesis of ((2R,4S,5R)-5-ethoxy-4-(ethylamino)tetrahydro-2H-pyran-2-yl)((S)-1-(4-fluorophenyl)-3,4-dihydroisoquinolin-2(1H)-yl)methanone (COMPOUND 5510)

HCl (6 M in 2-propanol, 1 mL, 6.00 mmol) was added to a solution of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(ethyl)carbamate (75 mg, 0.142 mmol) in 2-propanol (4 mL). After stirring overnight, the mixture was partioned between dichloromethane and saturated aqueous Na₂CO₃. The organic layers was separated, dried on Na₂SO₄, and evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1) to give ((2R,4S,5R)-5-ethoxy-4-(ethylamino)tetrahydro-2H-pyran-2-yl)((S)-1-(4-fluorophenyl)-3,4-dihydroisoquinolin-2(1/H)-yl)methanone (COMPOUND 5510).

LCMS: 99%, RT=1.19 min., (M+H)⁺=427 (method P).

### Example 104

### ((2R,4S,5R)-5-ethoxy-4-(methylamino)tetrahydro-2H-pyran-2-yl)((S)-1-(4-fluorophenyl)-3,4-dihydroisoquinolin-2(1H)-yl)methanone (COMPOUND 5509)

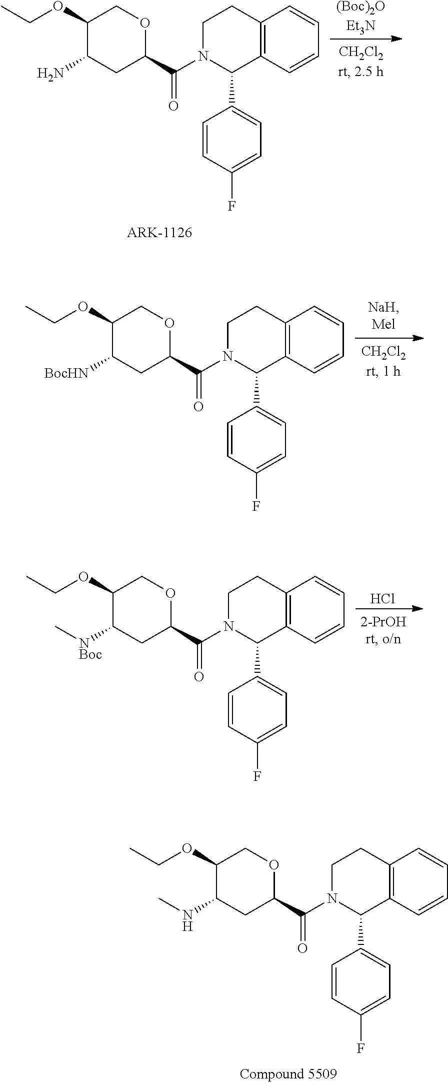

### Synthesis of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)carbamate

Triethylamine (0.063 mL, 0.452 mmol) and di-tert-butyl dicarbonate (99 mg, 0.452 mmol) were added to a solution of ((2R,4S,5R)-4-amino-5-ethoxytetrahydro-2H-pyran-2-yl)((S)-1-(4-fluorophenyl)-3,4-dihydroisoquinolin-2(1H)-yl)methanone (see Compound 5078, 150 mg, 0.376 mmol) in dichloromethane (3 mL). After 2.5 hours, the reaction mixture was concentrated to dryness under reduced pressure. The residue was purified by flash column chromatography (silica, 20 to 100% ethyl acetate in heptane) to give tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)carbamate.

LCMS: 99%, RT=2.20 min., (M+H)⁺=499 (method A).

### Synthesis of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(methyl)carbamate

Sodium hydride (60 wt % dispersion in mineral oil, 7.7 mg, 0.193 mmol) was added to a solution of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)carbamate (80 mg, 0.160 mmol) in N,N-dimethylformamide (3 mL). After 10 minutes, iodomethane (0.030 mL, 0.481 mmol) was added. After 1 hour, the mixture was diluted with water and extracted with ethyl acetate. The organic phase was evaporated under reduced pressure. The residue was purified by flash column chromatography (silica, 20 to 100% ethyl acetate in heptane) to give tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(methyl)carbamate.

LCMS: 98%, RT=2.31 min., (M+H)⁺=513 (method A).

### Synthesis of ((2R,4S,5R)-5-ethoxy-4-(methylamino)tetrahydro-2H-pyran-2-yl)((S)-1-(4-fluorophenyl)-3,4-dihydroisoquinolin-2(1H)-yl)methanone (COMPOUND 5509)

HCl (6 M in 2-propanol, 1 mL, 6.00 mmol) was added to a solution of tert-butyl ((2R,4S,5R)-5-ethoxy-2-((S)-1-(4-fluorophenyl)-1,2,3,4-tetrahydroisoquinoline-2-carbonyl)tetrahydro-2H-pyran-4-yl)(methyl)carbamate (80 mg, 0.156 mmol) in 2-propanol (4 mL). After stirring overnight, the mixture was partitioned between dichloromethane and saturated aqueous Na₂CO₃. The organic layer was separated, dried over Na₂SO₄, and evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1) to give ((2R,4S,5R)-5-ethoxy-4-(methylamino)tetrahydro-2H-pyran-2-yl)((S)-1-(4-fluorophenyl)-3,4-dihydroisoquinolin-2(1H)-yl)methanone (COMPOUND 5509).

LCMS: 98%, RT=1.16 min., (M+H)⁺=413 (method P).

### Example 105

### ((2R,4R,5S)-4-amino-5-methoxytetrahydro-2H-pyran-2-yl)((S)-1-(4-fluorophenyl)-3,4-dihydroisoquinolin-2(1H)-yl)methanone (COMPOUND 5247)

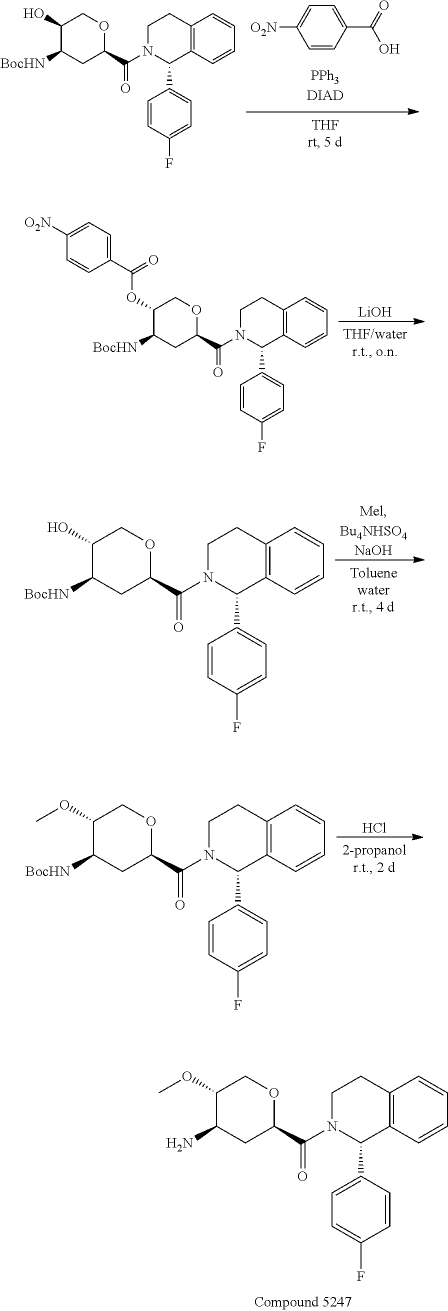

### Synthesis of (3S,4R,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate

Under a nitrogen atmosphere at 0° C., a solution of tert-butyl ((2R,4R,5R)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 100 mg, 0.213 mmol) and diisopropyl azodicarboxylate (0.050 mL, 0.255 mmol) in tetrahydrofuran (dry, 4 mL) was added to a solution of 4-nitrobenzoic acid (42.6 mg, 0.255 mmol) and triphenylphosphine (84 mg, 0.319 mmol) in tetrahydrofuran (dry, 5 mL). After the addition, the mixture was allowed to warm to room temperature and stirred overnight. Additional triphenylphosphine (84 mg, 0.319 mmol) and diisopropyl azodicarboxylate (0.050 mL, 0.255 mmol) were added and stirring was continued for 4 days. The reaction mixture was concentrated under reduced pressure. The residue was taken up in ethyl acetate (15 mL), washed with saturated aqueous NaHCO₃ (10 mL), dried on Na₂SO₄, and evaporated under reduced pressure. The residue was purified by flash column chromatography (silica, 0 to 40% ethyl acetate in heptane) to give (3S,4R,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate) as a mixture with diisopropyl hydrazine-1,2-dicarboxylate.

LCMS: 97%, RT=2.28 min., (M-Boc+H)⁺=520 (method B).

### Synthesis of tert-butyl ((2R,4R,5S)-2-((S)-1-(4-fluorophenyl)-1,2,3,4-tetrahydroisoquinoline-2-carbonyl)-5-hydroxytetrahydro-2H-pyran-4-yl)carbamate

A solution of (3S,4R,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl 4-nitrobenzoate (127 mg, 29 wt %, 0.059 mmol) as a mixture with diisopropyl hydrazine-1,2-dicarboxylate in tetrahydrofuran (2 mL) was added to a solution of lithium hydroxide monohydrate (4.99 mg, 0.119 mmol) in water (2 mL). After stirring overnight, the reaction mixture was diluted with ethyl acetate (10 mL) and washed with aqueous saturated NaHCO₃ (2 mL). The organic layer was dried on Na₂SO₄ and evaporated under reduced pressure. The residue was purified by flash column chromatography (silica, 0 to 100% ethyl acetate in heptane) to give tert-butyl ((2R,4R,5S)-2-((S)-1-(4-fluorophenyl)-1,2,3,4-tetrahydroisoquinoline-2-carbonyl)-5-hydroxytetrahydro-2H-pyran-4-yl)carbamate.

LCMS: 98%, RT=2.11 min., (M+Na)⁺=493 (method A).

### Synthesis of tert-butyl ((2R,4R,5S)-2-((S)-1-(4-fluorophenyl)-1,2,3,4-tetrahydroisoquinoline-2-carbonyl)-5-methoxytetrahydro-2H-pyran-4-yl)carbamate

Sodium hydroxide (50 wt % solution in water, 0.067 mL, 1.27 mmol), tetrabutylammonium hydrogen sulfate (5.05 mg, 0.015 mmol) and methyliodide (5.56 μL, 0.089 mmol) were added to a solution of tert-butyl ((2R,4R,5S)-2-((S)-1-(4-fluorophenyl)-1,2,3,4-tetrahydroisoquinoline-2-carbonyl)-5-hydroxytetrahydro-2H-pyran-4-yl)carbamate (14 mg, 0.030 mmol) in toluene (1.5 mL). After stirring overnight, additional methyliodide (1.85 μL, 0.030 mmol) was added. After 3 more days, the reaction mixture was diluted with water (1 mL) and dichloromethane (5 mL). The layers were separated using a phase-separator and the organic filtrate was evaporated under reduced pressure. The residue was purified by acidic preparative MPLC (Linear Gradient: t=0 min 5% A; t=1 min 30% A; t=16 min 70% A; t=17 min 100%; t=22 min 100% A; detection: 220/292 nm) to give tert-butyl ((2R,4R,5S)-2-((S)-1-(4-fluorophenyl)-1,2,3,4-tetrahydroisoquinoline-2-carbonyl)-5-methoxytetrahydro-2H-pyran-4-yl)carbamate.

LCMS: 98%, RT=2.18 min., (M+Na)⁺=507 (method A).

### Synthesis of ((2R,4R,5S)-4-amino-5-methoxytetrahydro-2H-pyran-2-yl)((S)-1-(4-fluorophenyl)-3,4-dihydroisoquinolin-2(1H)-yl)methanone (COMPOUND 5247)

HCl (5-6 M in 2-propanol, 0.5 mL, 2.75 mmol) was added to a solution of tert-butyl ((2R,4R,5S)-2-((S)-1-(4-fluorophenyl)-1,2,3,4-tetrahydroisoquinoline-2-carbonyl)-5-methoxytetrahydro-2H-pyran-4-yl)carbamate (8 mg, 0.017 mmol) in 2-propanol (3 mL). After 2 days, the mixture was partioned between dichloromethane (25 mL) and a mixture of saturated aqueous NaHCO₃ and water (1:1, 20 mL). The layers were separated and the aqueous phase was extracted with dichloromethane (25 mL). The combined organic layers were passed through a phase separator and evaporated under reduced pressure. The residue was dissolved in methanol, brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give ((2R,4R,5S)-4-amino-5-methoxytetrahydro-2H-pyran-2-yl)((S)-1-(4-fluorophenyl)-3,4-dihydroisoquinolin-2(1H)-yl)methanone (COMPOUND 5247).

LCMS: 99%, RT=1.06 min., (M+H)⁺=385 (method P).

### Example 106

### ((2R,4S,5R)-4-amino-5-(prop-2-yn-1-yloxy)tetrahydro-2H-pyran-2-yl)((S)-1-(4-fluorophenyl)-3,4-dihydroisoquinolin-2(1H)-yl)methanone (COMPOUND 5541)

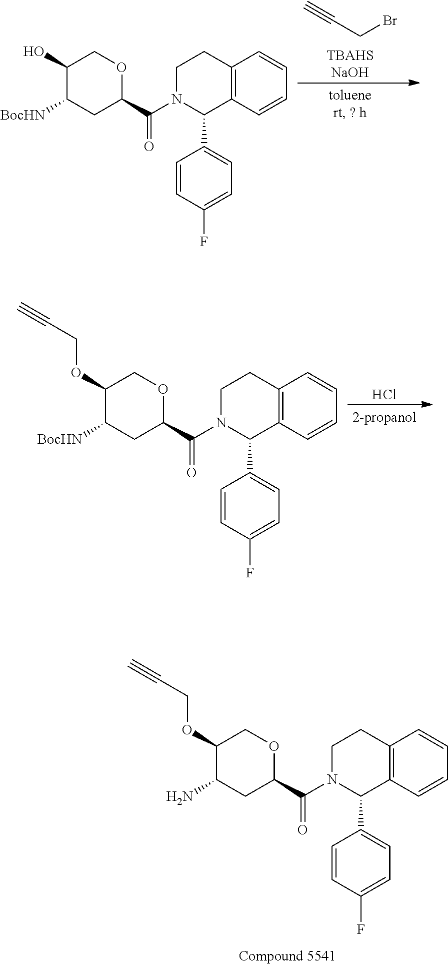

### Synthesis of tert-butyl ((2R,4S,5R)-2-((S)-1-(4-fluorophenyl)-1,2,3,4-tetrahydroisoquinoline-2-carbonyl)-5-(prop-2-yn-1-yloxy)tetrahydro-2H-pyran-4-yl)carbamate

Tetrabutylammonium hydrogen sulfate (22.95 mg, 0.068 mmol), sodium hydroxide (50 wt % solution in water, 0.180 mL, 3.38 mmol) and 3-bromoprop-1-yne (80 wt % in toluene, 0.036 mL, 0.338 mmol) were added to a solution of tert-butyl ((2R,4S,5R)-2-((S)-1-(4-fluorophenyl)-1,2,3,4-tetrahydroisoquinoline-2-carbonyl)-5-hydroxytetrahydro-2H-pyran-4-yl)carbamate (See Compound 5055, 106 mg, 0.225 mmol) in toluene (2 mL). After stirring overnight, additional sodium hydroxide (50 wt % solution in water, 0.180 mL, 3.38 mmol) and 3-bromoprop-1-yne (80 wt % in toluene, 0.036 mL, 0.338 mmol) were added and stirring was continued for 5 days. Then, the mixture was diluted with water (10 mL) and saturated aqueous NaHCO₃ (2 mL) and extracted with toluene (10 mL). The organic layer was dried on Na₂SO₄ and evaporated under reduced pressure. The residue was purified by flash column chromatography (silica, 10 to 50% ethyl acetate in heptane) to give tert-butyl ((2R,4S,5R)-2-((S)-1-(4-fluorophenyl)-1,2,3,4-tetrahydroisoquinoline-2-carbonyl)-5-(prop-2-yn-1-yloxy)tetrahydro-2H-pyran-4-yl)carbamate.

LCMS: 99%, RT=2.13 min., (M+H)⁺=509 (method A).

### Synthesis of ((2R,4S,5R)-4-amino-5-(prop-2-yn-1-yloxy)tetrahydro-2H-pyran-2-yl)((S)-1-(4-fluorophenyl)-3,4-dihydroisoquinolin-2(1H)-yl)methanone (COMPOUND 5541)

HCl (5 M in 2-propanol, 2.5 mL, 12.50 mmol) was added to a suspension of tert-butyl ((2R,4S,5R)-2-((S)-1-(4-fluorophenyl)-1,2,3,4-tetrahydroisoquinoline-2-carbonyl)-5-(prop-2-yn-1-yloxy)tetrahydro-2H-pyran-4-yl)carbamate (86.8 mg, 0.171 mmol) in 2-propanol (10 ml) in the dark. The mixture was stirred for 1.5 day and then diluted with water (10 mL) and saturated aqueous K₂CO₃ until pH is ca. 10-12 on pH paper. The volatiles were removed under reduced pressure and the aqueous residue was stirred with dichloromethane. After 0.5 hours, the layers were separated using a phase separator and the organic filtrate was concentrated under reduced pressure. The residue was lyophilised from a mixture of acetonitrile and water (1:1, 4 mL) to give ((2R,4S,5R)-4-amino-5-(prop-2-yn-1-yloxy)tetrahydro-2H-pyran-2-yl)((S)-1-(4-fluorophenyl)-3,4-dihydroisoquinolin-2(1H)-yl)methanone (COMPOUND 5541, 70 mg).

LCMS: 99%, RT=1.13 min., (M+H)⁺=409 (method P).

### Example 107

### N-((3S,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5427)

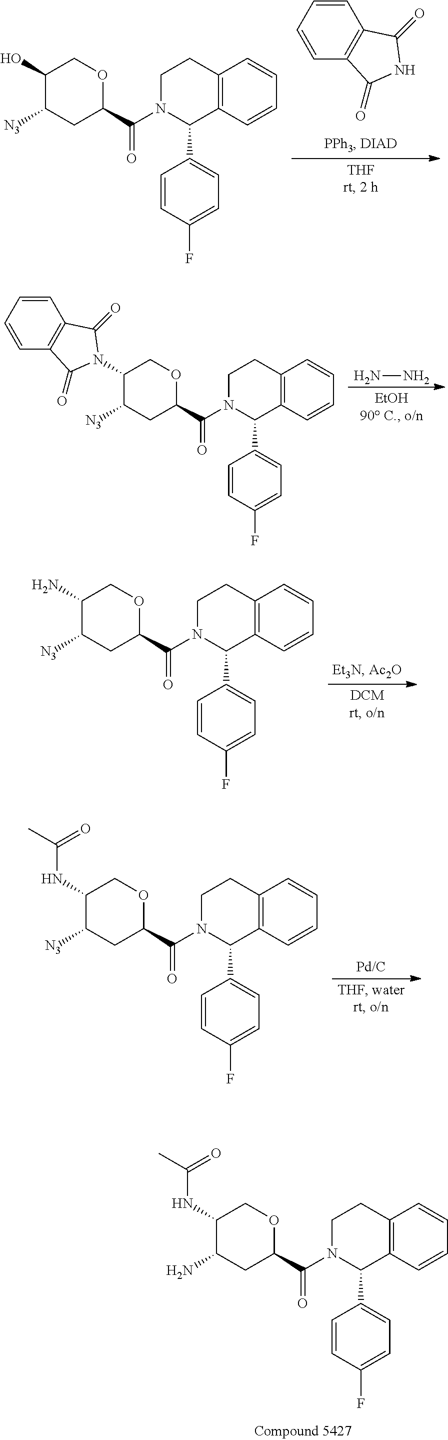

### Synthesis of 2-((3S,4S,6R)-4-azido-6-((S)-1-(4-fluorophenyl)-1,2,3,4-tetrahydroisoquinoline-2-carbonyl)tetrahydro-2H-pyran-3-yl)isoindoline-1,3-dione

Under nitrogen atmosphere at 0° C., diisopropyl azodicarboxylate (0.177 mL, 0.908 mmol) was added dropwise to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (300 mg, 0.757 mmol), triphenylphosphine (238 mg, 0.908 mmol), and phthalimide (134 mg, 0.908 mmol) in tetrahydrofuran (dry, 6 mL). After 10 minutes, the mixture was allowed to warm to room temperature and stirred for 2 hours. Then, the reaction mixture was concentrated to dryness under reduced pressure. The residue was purified by flash column chromatography (silica, 10 to 35% ethyl acetate in heptane) to give 2-((3S,4S,6R)-4-azido-6-((S)-1-(4-fluorophenyl)-1,2,3,4-tetrahydroisoquinoline-2-carbonyl)tetrahydro-2H-pyran-3-yl)isoindoline-1,3-dione. LCMS: 98%, RT=2.22 min., (M+H)⁺=526 (method A).

### Synthesis of ((2R,4S,5S)-5-amino-4-azidotetrahydro-2H-pyran-2-yl)((S)-1-(4-fluorophenyl)-3,4-dihydroisoquinolin-2(1H)-yl)methanone

Hydrazine monohydrate (0.036 mL, 0.736 mmol) was added to a solution of 2-((3S,4S,6R)-4-azido-6-((S)-1-(4-fluorophenyl)-1,2,3,4-tetrahydroisoquinoline-2-carbonyl)tetrahydro-2H-pyran-3-yl)isoindoline-1,3-dione (258 mg, 70 wt %, 0.344 mmol), as a mixture with diisopropyl hydrazine-1,2-dicarboxylate, in ethanol (5 mL). The mixture was stirred at 90° C. overnight. After cooling to room temperature, the mixture was filtered, and the filtrate was evaporated under reduced pressure. The residue was purified by flash column chromatography (silica, 2.5 to 10% methanol in dichloromethane) to give ((2R,4S,5S)-5-amino-4-azidotetrahydro-2H-pyran-2-yl)((S)-1-(4-fluorophenyl)-3,4-dihydroisoquinolin-2(1H)-yl)methanone.

LCMS: 96%, RT=1.67 min., (M+H)⁺=396 (method A).

### Synthesis of N-((3S,4S,6R)-4-azido-6-((S)-1-(4-fluorophenyl)-1,2,3,4-tetrahydroisoquinoline-2-carbonyl)tetrahydro-2H-pyran-3-yl)acetamide

Acetic anhydride (0.012 mL, 0.126 mmol) was added to a solution of ((2R,4S,5S)-5-amino-4-azidotetrahydro-2H-pyran-2-yl)((S)-1-(4-fluorophenyl)-3,4-dihydroisoquinolin-2(1H)-yl)methanone (40 mg, 0.101 mmol) and triethylamine (0.015 mL, 0.106 mmol) in dichloromethane (1 mL). After stirring overnight, the mixture was diluted with water (2 mL) and extracted with dichloromethane (3×1 mL). The combined extracts were passes through a phase separator and evaporated under reduced pressure. The residue was purified by flash column chromatography (silica, 2 to 6% methanol in dichloromethane) to give N-((3S,4S,6R)-4-azido-6-((S)-1-(4-fluorophenyl)-1,2,3,4-tetrahydroisoquinoline-2-carbonyl)tetrahydro-2H-pyran-3-yl)acetamide that was used as such.

LCMS: 99%, RT=1.99 min., (M+H)⁺=438 (method B).

### Synthesis of N-((3S,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5427)

A solution of N-((3S,4S,6R)-4-azido-6-((S)-1-(4-fluorophenyl)-1,2,3,4-tetrahydroisoquinoline-2-carbonyl)tetrahydro-2H-pyran-3-yl)acetamide (49 mg, 0.112 mmol) in a mixture of tetrahydrofuran (1 mL) and water (0.2 mL) was hydrogenated in the presence of palladium (10 wt % on carbon, containing 50% water, 23.84 mg, 0.011 mmol) and atmospheric hydrogen pressure. After stirring overnight, the reaction mixture was filtered and washed with dichloromethane. The combined filtrate and washings were evaporated under reduced pressure. The residue was purified by flash column chromatography (silica, 1 to 6% methanol in dichloromethane). The product was dissolved in methanol and brought onto an SCX-2 column (2 g) and eluted with methanol until neutral. Next, the column was eluted with ammonia in methanol (2.5 M). The basic fraction was concentrated to dryness under reduced pressure to give N-((3S,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5427) after lyophilization from a mixture of acetonitrile and water (1:1, 4 mL).

LCMS: 99%, RT=1.03 min., (M+H)⁺=412 (method P).

### Example 108

### N-((3S,4S,6R)-4-amino-6-((S)-1-(4-fluorophenyl)-1,2,3,4-tetrahydroisoquinoline-2-carbonyl)tetrahydro-2H-pyran-3-yl)methanesulfonamide (COMPOUND 5428)

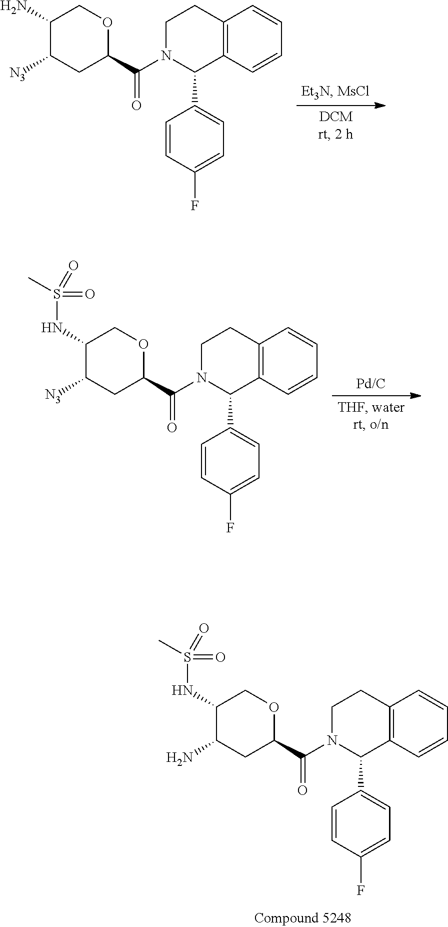 [SMILES: [N-]=[N+]=N[C@H]1C[C@H](C(=O)N2CCc3ccccc3[C@@H]2c2ccc(F)cc2)OC[C@H]1N ; CS(=O)(=O)N[C@@H]1CO[C@@H](C(=O)N2CCc3ccccc3[C@@H]2c2ccc(F)cc2)C[C@@H]1N=[N+]=[N-] ; CS(=O)(=O)N[C@@H]1CO[C@@H](C(=O)N2CCc3ccccc3[C@@H]2c2ccc(F)cc2)C[C@@H]1N]

### Synthesis of N-((3S,4S,6R)-4-azido-6-((S)-1-(4-fluorophenyl)-1,2,3,4-tetrahydroisoquinoline-2-carbonyl)tetrahydro-2H-pyran-3-yl)methanesulfonamide

Under argon atmosphere, methanesulfonyl chloride (0.020 ml, 0.253 mmol) was added dropwise to a solution of ((2R,4S,5S)-5-amino-4-azidotetrahydro-2H-pyran-2-yl)((S)-1-(4-fluorophenyl)-3,4-dihydroisoquinolin-2(1H)-yl)methanone (see Compound 5247, 50 mg, 0.126 mmol) and triethylamine (0.070 mL, 0.506 mmol) in dichloromethane (2.5 mL). After 2 hours, the mixture was basified with saturated aqueous NaHCO₃ and extracted with dichloromethane (3×2 mL). The combined organics were passed through a phase separator and evaporated under reduced pressure. The residue was purified by flash column chromatography (silica, 10 to 60% ethyl acetate in heptane) to give N-((3S,4S,6R)-4-azido-6-((S)-1-(4-fluorophenyl)-1,2,3,4-tetrahydroisoquinoline-2-carbonyl)tetrahydro-2H-pyran-3-yl)methanesulfonamide.

LCMS: 99%, RT=2.06 min., (M+H)⁺=474 (method A).

### Synthesis of N-((3S,4S,6R)-4-amino-6-((S)-1-(4-fluorophenyl)-1,2,3,4-tetrahydroisoquinoline-2-carbonyl)tetrahydro-2H-pyran-3-yl)methanesulfonamide (COMPOUND 5428)

A solution of N-((3S,4S,6R)-4-azido-6-((S)-1-(4-fluorophenyl)-1,2,3,4-tetrahydroisoquinoline-2-carbonyl)tetrahydro-2H-pyran-3-yl)methanesulfonamide (41 mg, 0.087 mmol) in a mixture of tetrahydrofuran (1.5 mL) and water (0.3 mL) was hydrogenated in the presence of palladium (10 wt % on carbon, containing 50% water, 36.9 mg, 0.017 mmol) and atmospheric hydrogen pressure. After stirring overnight, the reaction mixture was filtered and the filtrate was evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product was dissolved in methanol and brought onto an SCX-2 column (1 g) and eluted with methanol until neutral. Next, the column was eluted with ammonia in methanol (2 M). The basic fraction was concentrated to dryness under reduced pressure to give N-((3S,4S,6R)-4-amino-6-((S)-1-(4-fluorophenyl)-1,2,3,4-tetrahydroisoquinoline-2-carbonyl)tetrahydro-2H-pyran-3-yl)methanesulfonamide (COMPOUND 5428) after lyophilization from a mixture of acetonitrile and water (1:1, 4 mL).

LCMS: 99%, RT=1.02 min., (M+H)⁺=448 (method P).

### Example 109

### ((S)-1-(4-fluorophenyl)-3,4-dihydroisoquinolin-2(1H)-yl)((2R,4S,5S)-5-hydroxy-4-(methylamino)tetrahydro-2H-pyran-2-yl)methanone (COMPOUND 5444)

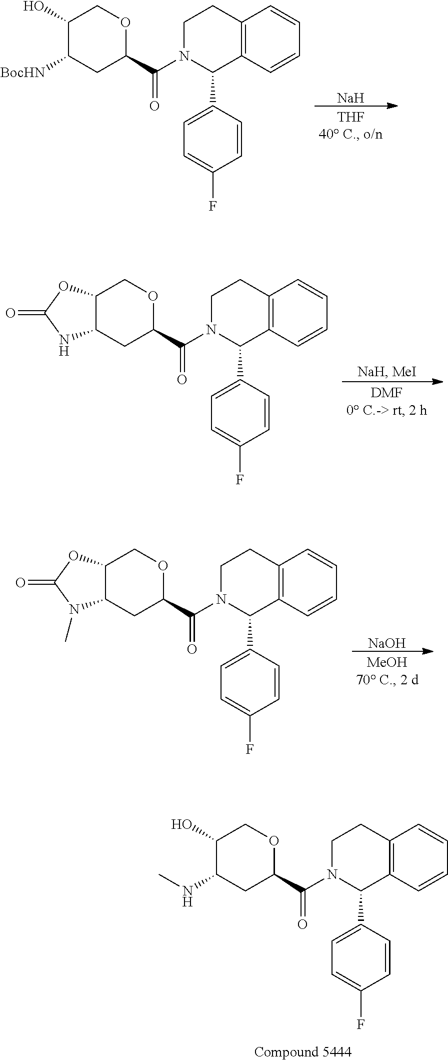

### Synthesis of (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one

Under argon atmosphere, sodium hydride (60 wt % dispersion in mineral oil, 25.5 mg, 0.638 mmol) was added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 300 mg, 0.638 mmol) in tetrahydrofuran (dry, 15 mL) and the reaction mixture was warmed to 40° C. After stirring overnight, the reaction mixture was diluted with dichloromethane (25 mL) and saturated aqueous NH₄Cl (20 mL). The layers were separated, and the aqueous phase was extracted with dichloromethane (2×25 mL). The combined organics were dried over Na₂SO₄ and evaporated under reduced pressure. The residue was purified by flash column chromatography (silica, 40 to 100% ethyl acetate in heptane) to give (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one.

LCMS: 99%, RT=2.07 min., (M+H)⁺=397 (method A).

### Synthesis of (3aS,6R,7aS)-6-((S)-1-(4-fluorophenyl)-1,2,3,4-tetrahydroisoquinoline-2-carbonyl)-1-methylhexahydro-2H-pyrano[4,3-d]oxazol-2-one

Under argon atmosphere at 0° C., sodium hydride (60 wt % dispersion in mineral oil, 22.40 mg, 0.560 mmol) was added to a solution of (3aS,6R,7aS)-6-((S)-1-(4-fluorophenyl)-1,2,3,4-tetrahydroisoquinoline-2-carbonyl)hexahydro-2H-pyrano[4,3-d]oxazol-2-one (185 mg, 0.467 mmol) in N,N-dimethylformamide (dry, 4.5 mL) After 15 minutes, iodomethane (0.157 mL, 2.52 mmol) was added. After 5 hours, the reaction mixture was diluted with water (15 mL) and extracted with ethyl acetate (3×15 mL). The combined organics were washed with brine (10 mL), dried on Na₂SO₄, and evaporated under reduced pressure. The residue was purified by flash column chromatography (silica, 30 to 100% ethyl acetate in heptane). The product was taken up in ethyl acetate (5 mL), washed with brine (5 mL), dried on Na₂SO₄, and evaporated under reduced pressure to give (3aS,6R,7aS)-6-((S)-1-(4-fluorophenyl)-1,2,3,4-tetrahydroisoquinoline-2-carbonyl)-1-methylhexahydro-2H-pyrano[4,3-d]oxazol-2-one LCMS: 99%, RT=1.99 min., (M+H)⁺=411 (method A).

### Synthesis of ((S)-1-(4-fluorophenyl)-3,4-dihydroisoquinolin-2(1H)-yl)((2R,4S,5S)-5-hydroxy-4-(methylamino)tetrahydro-2H-pyran-2-yl)methanone (COMPOUND 5444

Aqueous NaOH (2 M, 1.89 mL, 3.78 mmol) was added to a solution of (3aS,6R,7aS)-6-((S)-1-(4-fluorophenyl)-1,2,3,4-tetrahydroisoquinoline-2-carbonyl)-1-methylhexahydro-2H-pyrano[4,3-d]oxazol-2-one (155 mg, 0.378 mmol) in methanol (17 mL) and the mixture was warmed to 70° C. After 4 days, the reaction mixture was concentrated under reduced pressure. The residue was diluted with dichloromethane (15 mL) and saturated aqueous NaHCO₃ (20 mL). The aqueous layer was separated and extracted with dichloromethane (3×15 mL).

The combined organics were dried on Na₂SO₄ and evaporated under reduced pressure. The residue was purified by flash column chromatography (silica, 0 to 5% (7M ammonia in methanol) in dichloromethane) and preparative SFC (method BW) to give ((S)-1-(4-fluorophenyl)-3,4-dihydroisoquinolin-2(1H)-yl)((2R,4S,5S)-5-hydroxy-4-(methylamino)tetrahydro-2H-pyran-2-yl)methanone after lyophilization from a mixture of acetonitrile and water (1:1, 4 mL).

LCMS: 99%, RT=1.03 min., (M+H)⁺=385 (method P). SFC: 100%, RT=3.00 min., (M+H)⁺=385 (method AU).

### Example 110

### ((2R,4S,5R)-4-amino-5-(tert-butoxy)tetrahydro-2H-pyran-2-yl)((S)-1-(4-fluorophenyl)-3,4-dihydroisoquinolin-2(1H)-yl)methanone (COMPOUND 5511)

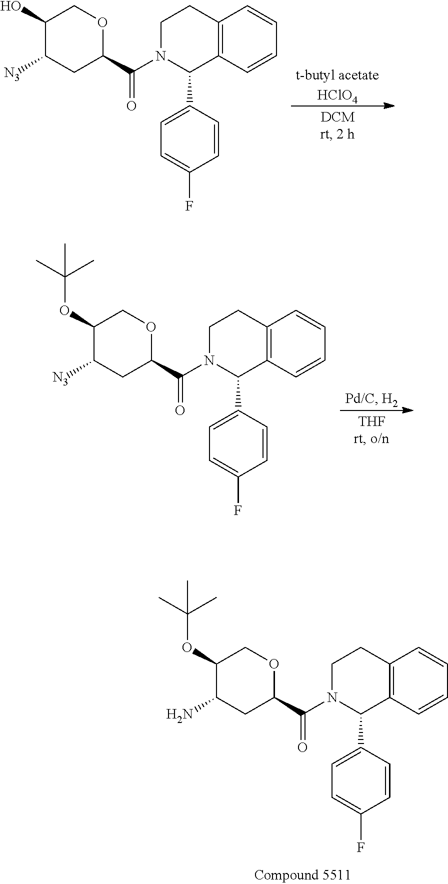 [SMILES: [N-]=[N+]=N[C@H]1C[C@H](C(=O)N2CCc3ccccc3[C@@H]2c2ccc(F)cc2)OC[C@@H]1O ; "t-butyl acetateHClO4" ; CC(C)(C)O[C@H]1CO[C@@H](C(=O)N2CCc3ccccc3[C@@H]2c2ccc(F)cc2)C[C@@H]1N=[N+]=[N-] ; "Pd/C, H2" ; CC(C)(C)O[C@H]1CO[C@@H](C(=O)N2CCc3ccccc3[C@@H]2c2ccc(F)cc2)C[C@@H]1N]

### Synthesis of ((2R,4S,5R)-4-azido-5-(tert-butoxy)tetrahydro-2H-pyran-2-yl)((S)-1-(4-fluorophenyl)-3,4-dihydroisoquinolin-2(1H)-yl)methanone

Perchloric acid (0.017 mL, 0.202 mmol) was added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 100 mg, 0.252 mmol) in a mixture of tert-butyl acetate (2 mL, 14.98 mmol) and dichloromethane (3 mL). After 2 hours, the mixture was diluted with dichloromethane (4 mL) and saturated aqueous NaHCO₃ (4 mL). The layers were separated using a phase-separator and the organic filtrate was evaporated under reduced pressure. The residue was purified by flash column chromatography (silica, 0 to 100% ethyl acetate in heptane) to give ((2R,4S,5R)-4-azido-5-(tert-butoxy)tetrahydro-2H-pyran-2-yl)((S)-1-(4-fluorophenyl)-3,4-dihydroisoquinolin-2(1H)-yl)methanone.

LCMS: 99%, RT=2.40 min., (M+H)⁺=453 (method A).

### Synthesis of ((2R,4S,5R)-4-amino-5-(tert-butoxy)tetrahydro-2H-pyran-2-yl)((S)-1-(4-fluorophenyl)-3,4-dihydroisoquinolin-2(1H)-yl)methanone (COMPOUND 5511)

A solution of ((2R,4S,5R)-4-azido-5-(tert-butoxy)tetrahydro-2H-pyran-2-yl)((S)-1-(4-fluorophenyl)-3,4-dihydroisoquinolin-2(1H)-yl)methanone (53 mg, 0.117 mmol) in tetrahydrofuran (3 mL) was hydrogenated in the presence of palladium (10 wt % on carbon, containing 50% water, 24.9 mg, 0.012 mmol) and atmospheric hydrogen pressure. After stirring overnight, the reaction mixture was filtered and washed with methanol. The combined filtrate and washings were evaporated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give ((2R,4S,5R)-4-amino-5-(tert-butoxy)tetrahydro-2H-pyran-2-yl)((S)-1-(4-fluorophenyl)-3,4-dihydroisoquinolin-2(1H)-yl)methanone (COMPOUND 5511).

LCMS: 97%, RT=1.22 min., (M+H)⁺=427 (method P).

### Example 111

### ((S)-1-(4-fluorophenyl)-3,4-dihydroisoquinolin-2(1H)-yl)((4aR,7R,8aS)-octahydropyrano[3,4-b][1,4]oxazin-7-yl)methanone (COMPOUND 5266)

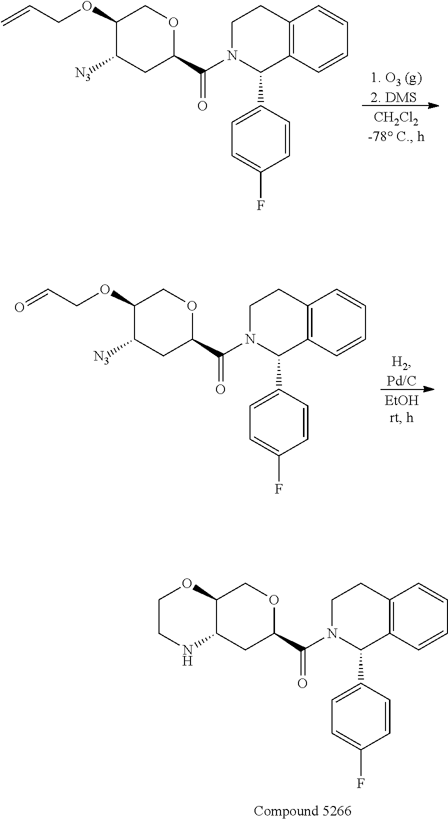

### Synthesis of 2-(((3R,4S,6R)-4-azido-6-((S)-1-(4-fluorophenyl)-1,2,3,4-tetrahydroisoquinoline-2-carbonyl)tetrahydro-2H-pyran-3-yl)oxy)acetaldehyde

At −78° C., a mixture of ozone and oxygen was bubbled through a solution of ((2R,4S,5R)-5-(allyloxy)-4-azidotetrahydro-2H-pyran-2-yl)((S)-1-(4-fluorophenyl)-3,4-dihydroisoquinolin-2(1H)-yl)methanone (see Compound 5474, 400 mg, 0.916 mmol) in dichloromethane (20 mL) till the reaction mixture turned pale blue (2 minutes). After 5 minutes, the reaction mixture was purged with argon gas for 5 minutes. Then, dimethyl sulfide (0.339 mL, 4.58 mmol) was added and after 1 hour the mixture was allowed to warm to room temperature and stirred overnight. The reaction mixture was combined with the reaction mixture of the same conversion (starting from 0.206 mmol) and evaporated under reduced pressure.

The residue was purified by flash column chromatography (silica, 0 to 100% ethyl acetate in heptane) to give 2-(((3R,4S,6R)-4-azido-6-((S)-1-(4-fluorophenyl)-1,2,3,4-tetrahydroisoquinoline-2-carbonyl)tetrahydro-2H-pyran-3-yl)oxy)acetaldehyde.

LCMS: 97%, RT=plateau between 1.9-2.2 min., (M+H)⁺=439 (method B).

### Synthesis of ((S)-1-(4-fluorophenyl)-3,4-dihydroisoquinolin-2(1H)-yl)((4aR,7R,8aS)-octahydropyrano[3,4-b][1,4]oxazin-7-yl)methanone (COMPOUND 5266)

A solution of 2-(((3R,4S,6R)-4-azido-6-((S)-1-(4-fluorophenyl)-1,2,3,4-tetrahydroisoquinoline-2-carbonyl)tetrahydro-2H-pyran-3-yl)oxy)acetaldehyde (320 mg, 0.73 mmol) in ethanol (35 mL) was hydrogenated in the presence of palladium (10 wt % on carbon, containing 50% water, 350 mg, 0.164 mmol) and atmospheric hydrogen pressure. After 5 hours, the reaction mixture was filtered and washed with ethanol. The combined filtrate and washings were evaporated under reduced pressure. The residue was purified by flash column chromatography (silica, 0.5 to 5% (7M ammonia in methanol) in dichloromethane) and acidic preparative MPLC (Linear Gradient: t=0 min 5% A; t=1 min 5% A; t=2 min 10% A; t=17 min 50% A; t=18 min 100%; t=23 min 100% A; detection: 220 nm). The product containing fractions were pooled, diluted with saturated aqueous K₂CO₃ (2.5 mL), and partially concentrated to remove the acetonitrile. The aqueous residue was extracted with dichloromethane (2×10 mL). The combined extracts were dried on Na₂SO₄ and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((4aR,7R,8aS)-octahydropyrano[3,4-b][1,4]oxazin-7-yl)methanone (COMPOUND 5266).

LCMS: 96%, RT=2.75 min., (M+H)⁺=397 (method AK).

### Example 112

### N-((3R,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5512)

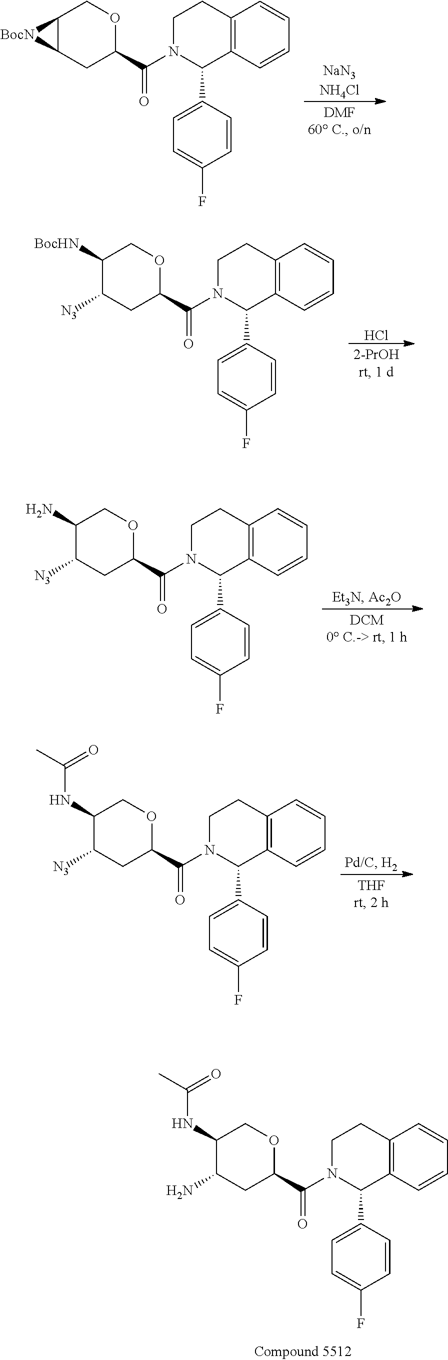

### Synthesis of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate

Ammonium chloride (0.284 g, 5.30 mmol) and sodium azide (0.517 g, 7.96 mmol) were added to a solution of tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate (see Compound 5210, 1.20 g, 2.65 mmol) in N,N-dimethylformamide (dry, 13 mL). The mixture was warmed 60° C. and stirred overnight. Then, the mixture was diluted with brine (20 mL) and extracted with ethyl acetate (2×60 mL). The combined organic layers were washed with brine (3×20 mL), dried on Na₂SO₄, and evaporated under reduced pressure. The residue was purified by flash column chromatography (silica, 10 to 40% ethyl acetate in heptane) to give tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate.

LCMS: 99%, RT=2.26 min., (M+H)⁺=496 (method A).

### Synthesis of ((2R,4S,5R)-5-amino-4-azidotetrahydro-2H-pyran-2-yl)((S)-1-(4-fluorophenyl)-3,4-dihydroisoquinolin-2(1H)-yl)methanone

At 0° C., HCl (5-6 M in 2-propanol, 6.05 mL, 33.3 mmol) was added to a solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate (550 mg, 1.110 mmol) in 2-propanol (11 mL). After 10 minutes, the cooling bath was removed and the reaction mixture was stirred for 1 day. The mixture was diluted with saturated aqueous Na₂CO₃ (18 mL) and brine (30 mL) and extracted with dichloromethane (100 mL). The organic layer was washed with a mixture of saturated aqueous Na₂CO₃ and brine (1:1, 30 mL), dried over Na₂SO₄, and evaporated under reduced pressure to give ((2R,4S,5R)-5-amino-4-azidotetrahydro-2H-pyran-2-yl)((S)-1-(4-fluorophenyl)-3,4-dihydroisoquinolin-2(1H)-yl)methanone.

LCMS: 98%, RT=1.88 min., (M+H)⁺=396 (method A).

### Synthesis of N-((3R,4S,6R)-4-azido-6-((S)-1-(4-fluorophenyl)-1,2,3,4-tetrahydroisoquinoline-2-carbonyl)tetrahydro-2H-pyran-3-yl)acetamide

Under nitrogen atmosphere, acetic anhydride (0.023 mL, 0.243 mmol) was added to a solution of ((2R,4S,5R)-5-amino-4-azidotetrahydro-2H-pyran-2-yl)((S)-1-(4-fluorophenyl)-3,4-dihydroisoquinolin-2(1H)-yl)methanone (80 mg, 0.202 mmol) and triethylamine (0.037 mL, 0.263 mmol) in dichloromethane (dry, 1 mL). After 1 hour, the mixture was purified by flash column chromatography (silica, 10 to 60% ethyl acetate in heptane) to give N-((3R,4S,6R)-4-azido-6-((S)-1-(4-fluorophenyl)-1,2,3,4-tetrahydroisoquinoline-2-carbonyl)tetrahydro-2H-pyran-3-yl)acetamide.

LCMS: 99%, RT=2.03 min., (M+H)⁺=438 (method A).

### Synthesis of N-((3R,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5512)

A solution of N-((3R,4S,6R)-4-azido-6-((S)-1-(4-fluorophenyl)-1,2,3,4-tetrahydroisoquinoline-2-carbonyl)tetrahydro-2H-pyran-3-yl)acetamide (76 mg, 0.174 mmol) in tetrahydrofuran (1.7 mL) was hydrogenated in the presence of palladium (10 wt % on carbon, containing 50% water, 37.0 mg, 0.017 mmol) at atmospheric hydrogen pressure. After 2 hours, the mixture was filtered over a 45 μm nylon filter and washed with ethyl acetate. The combined filtrates were concentrated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:4, 2 mL) to give N-((3R,4S,6R)-4-amino-6-((S)-1-(4-fluorophenyl)-1,2,3,4-tetrahydroisoquinoline-2-carbonyl)tetrahydro-2H-pyran-3-yl)acetamide (COMPOUND 5512).

LCMS: 99%, RT=1.04 min., (M+H)⁺=412 (method P).

### Example 113

### N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylacetamide (COMPOUND 5516)

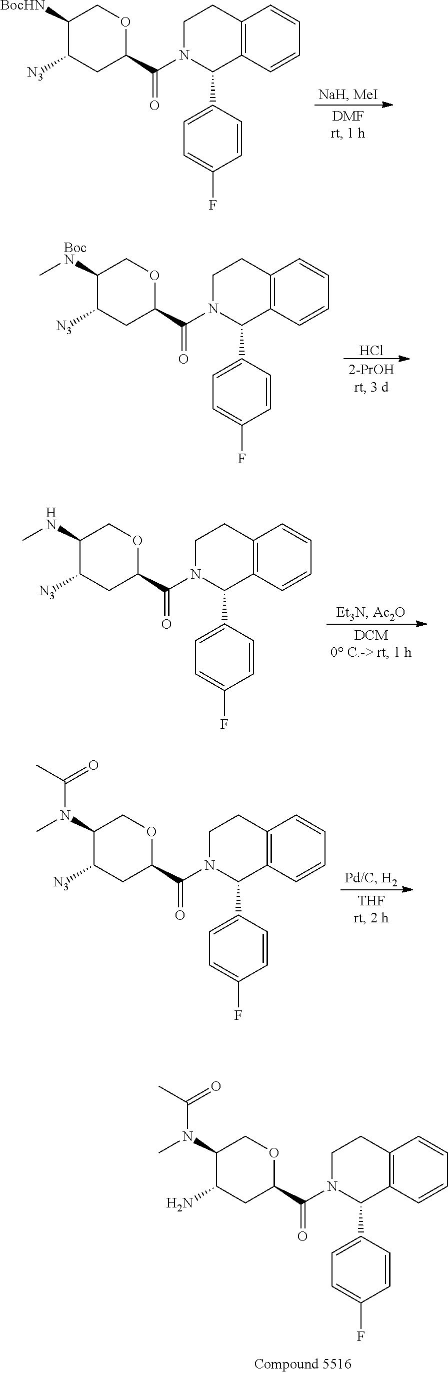

### Synthesis of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate

Under nitrogen atmosphere, sodium hydride (60 wt % dispersion in mineral oil, 38.7 mg, 0.969 mmol) was added to a solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate (see COMPOUND 5512400 mg, 0.807 mmol) in N,N-dimethylformamide (dry, 4.0 mL). After 15 minutes, methyl iodide (0.176 mL, 2.83 mmol) was added. After 1 hour, the mixture was partitioned between ethyl acetate (30 mL) and brine (10 mL). The aqueous layer was extracted with ethyl acetate (20 mL) and the combined organic layers were washed with brine (3×10 mL), dried on Na₂SO₄, and evaporated under reduced pressure. The residue was purified by flash column chromatography (silica, 5 to 25% ethyl acetate in heptane) to give tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate.

LCMS: 99%, RT=2.35 min., (M+H)⁺=510 (method A).

### Synthesis of ((2R,4S,5R)-4-azido-5-(methylamino)tetrahydro-2H-pyran-2-yl)((S)-1-(4-fluorophenyl)-3,4-dihydroisoquinolin-2(1H)-yl)methanone

HCl (5-6 M in 2-propanol, 2.51 ml, 13.82 mmol) was added to a solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate (234 mg, 0.459 mmol) in a mixture of 2-propanol (8 mL) and dichloromethane (3 mL). After 3 days, the reaction mixture was diluted with dichloromethane (15 mL) and washed with saturated aqueous NaHCO₃ (25 mL). The aqueous phase was extracted with dichloromethane (3×20 mL) and the combined organics were dried over Na₂SO₄ and evaporated under reduced pressure. The residue was dissolved in methanol (2 mL) and brought onto an SCX-2 column (2 g) and eluted with methanol until neutral. Next, the column was eluted with ammonia in methanol (7 M). The basic fraction was concentrated to dryness under reduced pressure to give ((2R,4S,5R)-4-azido-5-(methylamino)tetrahydro-2H-pyran-2-yl)((S)-1-(4-fluorophenyl)-3,4-dihydroisoquinolin-2(1H)-yl)methanone.

LCMS: 99%, RT=1.76 min., (M+H)⁺=410 (method A).

### Synthesis of N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylacetamide

Under nitrogen atmosphere, acetic anhydride (0.028 mL, 0.302 mmol) was added to a solution of ((2R,4S,5R)-4-azido-5-(methylamino)tetrahydro-2H-pyran-2-yl)((S)-1-(4-fluorophenyl)-3,4-dihydroisoquinolin-2(1H)-yl)methanone (85 mg, 0.201 mmol) and triethylamine (0.039 mL, 0.282 mmol) in dichloromethane (dry, 1 mL). After 1 hour, the mixture was purified by flash column chromatography (silica, 25 to 65% ethyl acetate in heptane) to give N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylacetamide.

LCMS: 98%, RT=2.03 min., (M+H)⁺=452 (method A).

### Synthesis of N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylacetamide (COMPOUND 5516)

A solution of N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylacetamide (92.5 mg, 0.205 mmol) in tetrahydrofuran (2.0 mL) was hydrogenated in the presence of palladium (10 wt % on carbon, containing 50% water, 43.6 mg, 0.020 mmol) at atmospheric hydrogen pressure. After 2 hours, the mixture was filtered over a 45 μm nylon filter and washed with ethyl acetate. The combined filtrates were concentrated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (3:1, 3 mL) to give N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylacetamide (COMPOUND 5516).

LCMS: 99%, RT=1.06 min. (M+H)⁺=426 (method P).

### Example 114

### N-((3R,4S,6R)-4-amino-6-((S)-1-(4-fluorophenyl)-1,2,3,4-tetrahydroisoquinoline-2-carbonyl)tetrahydro-2H-pyran-3-yl)-2,2,2-trifluoroacetamide (COMPOUND 5514)

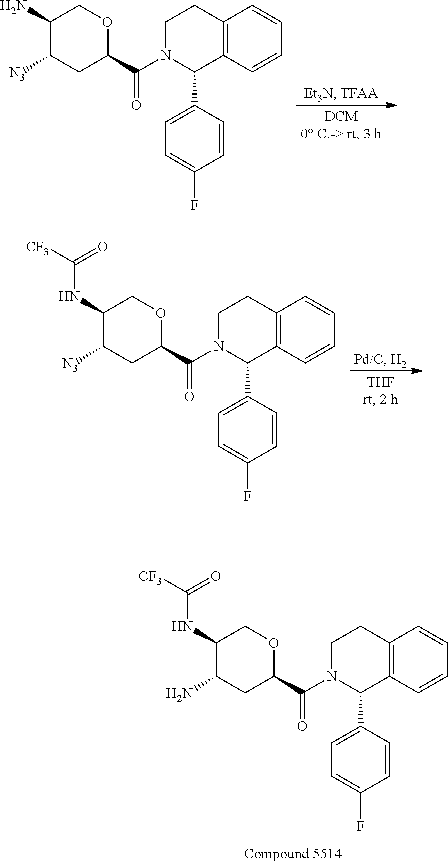

### Synthesis of N-((3R,4S,6R)-4-azido-6-((S)-1-(4-fluorophenyl)-1,2,3,4-tetrahydroisoquinoline-2-carbonyl)tetrahydro-2H-pyran-3-yl)-2,2,2-trifluoroacetamide

Under nitrogen atmosphere at 0° C., trifluoroacetic anhydride (0.034 mL, 0.243 mmol) was added to a solution of ((2R,4S,5R)-5-amino-4-azidotetrahydro-2H-pyran-2-yl)((S)-1-(4-fluorophenyl)-3,4-dihydroisoquinolin-2(1H)-yl)methanone (see COMPOUND 5512, 80 mg, 0.202 mmol) and triethylamine (0.037 mL, 0.263 mmol) in dichloromethane (dry, 1.0 mL). After 5 minutes, the reaction mixture was warmed to room temperature and stirred for 2.5 hour. Then, another portion of triethylamine (0.018 mL, 0.131 mmol) and trifluoroacetic anhydride (0.017 mL, 0.121 mmol) was added and stirring was continued for 0.5 hours. The mixture was diluted with dichloromethane (4 mL), washed with saturated aqueous NaHCO₃ (1 mL), dried over Na₂SO₄, and evaporated under reduced pressure. The residue was purified by flash column chromatography (silica, 05 to 40% ethyl acetate in heptane) to give N-((3R,4S,6R)-4-azido-6-((S)-1-(4-fluorophenyl)-1,2,3,4-tetrahydroisoquinoline-2-carbonyl)tetrahydro-2H-pyran-3-yl)-2,2,2-trifluoroacetamide.

LCMS: 99%, RT=2.36 min., (M+H)⁺=492 (method A).

### Synthesis of N-((3R,4S,6R)-4-amino-6-((S)-1-(4-fluorophenyl)-1,2,3,4-tetrahydroisoquinoline-2-carbonyl)tetrahydro-2H-pyran-3-yl)-2,2,2-trifluoroacetamide (COMPOUND 5514)

A solution of N-((3R,4S,6R)-4-azido-6-((S)-1-(4-fluorophenyl)-1,2,3,4-tetrahydroisoquinoline-2-carbonyl)tetrahydro-2H-pyran-3-yl)-2,2,2-trifluoroacetamide (93 mg, 0.189 mmol) in tetrahydrofuran (1.9 mL) was hydrogenated in the presence of palladium (10 wt % on carbon, containing 50% water, 40.3 mg, 0.019 mmol) at atmospheric hydrogen pressure. After 2 hours, the mixture was filtered over a 45 μm nylon filter and washed with ethyl acetate. The combined filtrates were concentrated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (3:2, 3 mL) to give N-((3R,4S,6R)-4-amino-6-((S)-1-(4-fluorophenyl)-1,2,3,4-tetrahydroisoquinoline-2-carbonyl)tetrahydro-2H-pyran-3-yl)-2,2,2-trifluoroacetamide (COMPOUND 5514).

LCMS: 97%, RT=1.15 min., (M+H)⁺=466 (method P).

### Example 115

### N-((3R,4S,6R)-4-amino-6-((S)-1-(4-fluorophenyl)-1,2,3,4-tetrahydroisoquinoline-2-carbonyl)tetrahydro-2H-pyran-3-yl)propionamide (COMPOUND 5513)

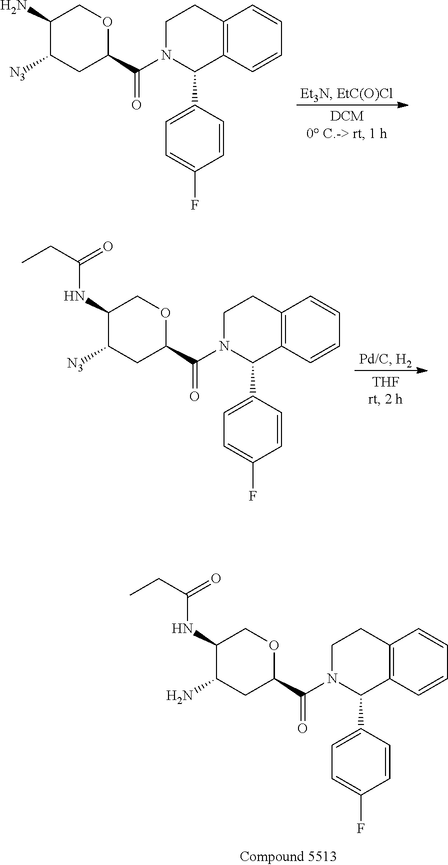

### Synthesis of N-((3R,4S,6R)-4-azido-6-((S)-1-(4-fluorophenyl)-1,2,3,4-tetrahydroisoquinoline-2-carbonyl)tetrahydro-2H-pyran-3-yl)propionamide

Under nitrogen atmosphere, propionic anhydride (0.034 mL, 0.263 mmol) was added to a solution of ((2R,4S,5R)-5-amino-4-azidotetrahydro-2H-pyran-2-yl)((S)-1-(4-fluorophenyl)-3,4-dihydroisoquinolin-2(1H)-yl)methanone (see COMPOUND 5512, 80 mg, 0.202 mmol) and triethylamine (0.039 mL, 0.283 mmol) in dichloromethane (dry, 1.0 mL). After 1 hour, the mixture was purified by flash column chromatography (silica, 5 to 60% ethyl acetate in heptane) to give N-((3R,4S,6R)-4-azido-6-((S)-1-(4-fluorophenyl)-1,2,3,4-tetrahydroisoquinoline-2-carbonyl)tetrahydro-2H-pyran-3-yl)propionamide.

LCMS: 99%, RT=2.25 min., (M+H)⁺=452 (method A).

### Synthesis of N-((3R,4S,6R)-4-amino-6-((S)-1-(4-fluorophenyl)-1,2,3,4-tetrahydroisoquinoline-2-carbonyl)tetrahydro-2H-pyran-3-yl)propionamide (COMPOUND 5513)

A solution of N-((3R,4S,6R)-4-azido-6-((S)-1-(4-fluorophenyl)-1,2,3,4-tetrahydroisoquinoline-2-carbonyl)tetrahydro-2H-pyran-3-yl)propionamide (82 mg, 0.182 mmol) in tetrahydrofuran (1.8 mL) was hydrogenated in the presence of palladium (10 wt % on carbon, containing 50% water, 38.7 mg, 0.018 mmol) at atmospheric hydrogen pressure. After 2 hours, the mixture was filtered over a 45 μm nylon filter and washed with ethyl acetate. The combined filtrates were concentrated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 3 mL) to give N-((3R,4S,6R)-4-amino-6-((S)-1-(4-fluorophenyl)-1,2,3,4-tetrahydroisoquinoline-2-carbonyl)tetrahydro-2H-pyran-3-yl)propionamide (COMPOUND 5513).

LCMS: 99%, RT=1.07 min., (M+H)⁺=426 (method P).

### Example 116

### N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylpropionamide (COMPOUND 5517)

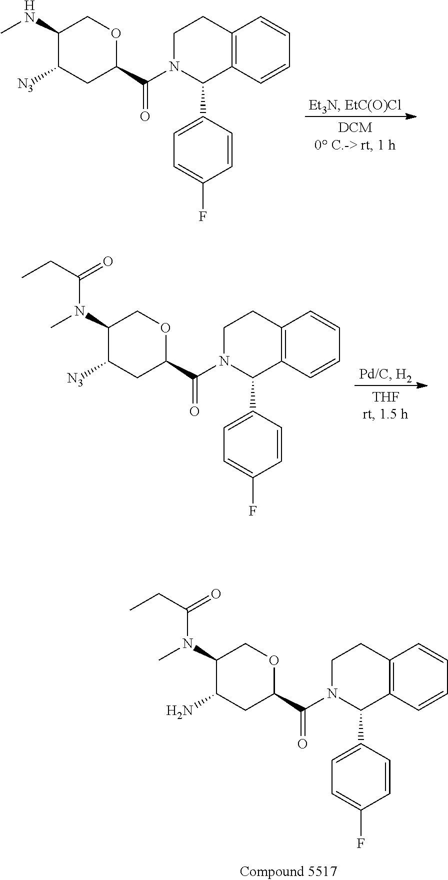

### Synthesis of N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylpropionamide

Under nitrogen atmosphere, propionic anhydride (0.034 mL, 0.262 mmol) was added to a solution of ((2R,4S,5R)-4-azido-5-(methylamino)tetrahydro-2H-pyran-2-yl)((S)-1-(4-fluorophenyl)-3,4-dihydroisoquinolin-2(1H)-yl)methanone (see COMPOUND 5516, 85 mg, 0.201 mmol) and triethylamine (0.039 mL, 0.282 mmol) in dichloromethane (dry, 1 mL). After 1 hour, the mixture was purified by flash column chromatography (silica, 15 to 55% ethyl acetate in heptane) to give N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylpropionamide.

LCMS: 99%, RT=2.10 min., (M+H)⁺=466 (method A).

### Synthesis of N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylpropionamide (COMPOUND 5517)

A solution of N-((3R,4S,6R)-4-azido-6-((S)-1-(4-fluorophenyl)-1,2,3,4-tetrahydroisoquinoline-2-carbonyl)tetrahydro-2H-pyran-3-yl)-N-methylpropionamide (82.5 mg, 0.177 mmol) in tetrahydrofuran (1.8 mL) was hydrogenated in the presence of palladium (10 wt % on carbon, containing 50% water, 37.7 mg, 0.018 mmol) at atmospheric hydrogen pressure. After 1.5 hours, the mixture was filtered over a 45 μm nylon filter and washed with ethyl acetate. The combined filtrates were concentrated under reduced pressure. The residue was dissolved in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (6:1, 3 mL) to give N-((3R,4S,6R)-4-amino-6-((S)-1-(4-fluorophenyl)-1,2,3,4-tetrahydroisoquinoline-2-carbonyl)tetrahydro-2H-pyran-3-yl)-N-methylpropionamide (COMPOUND 5517).

LCMS: 99%, RT=1.10 min., (M+H)⁺=440 (method P).

### Example 117

### ((S)-1-(4-fluorophenyl)-3,4-dihydroisoquinolin-2(1H)-yl)((3aS,6R,7aS)-1,3a,4,6,7,7a-hexahydropyrano[3,4-d]imidazol-6-yl)methanone (COMPOUND 5430)

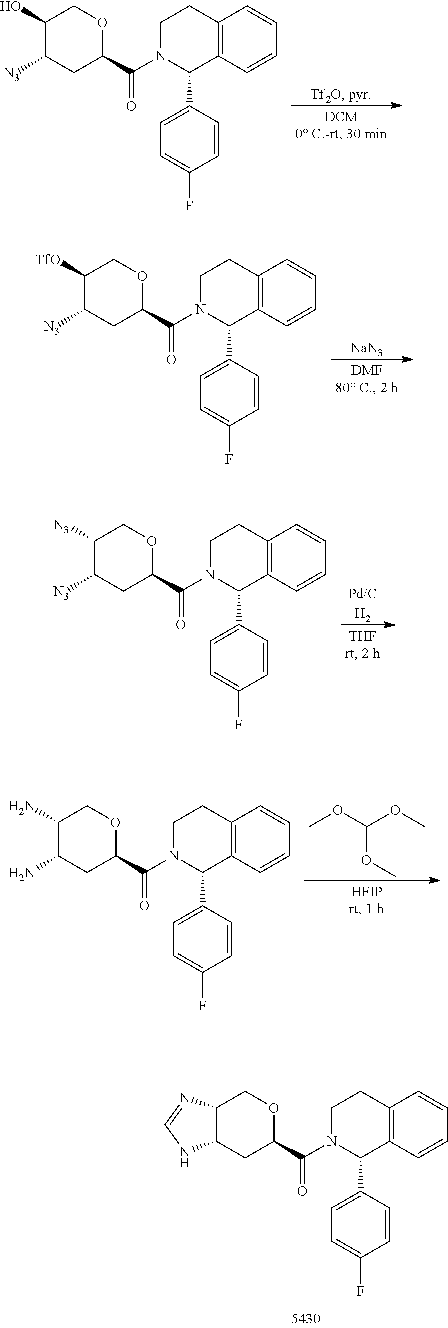

### Synthesis of (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate

Under argon atmosphere at 0° C., trifluoromethanesulfonic anhydride (0.314 mL, 1.892 mmol) was added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 0.50 g, 1.26 mmol) and pyridine (0.306 mL, 3.78 mmol) in dichloromethane (11 mL). After 30 minutes, the reaction mixture was diluted with dichloromethane (10 mL), washed with aqueous citric acid (0.5M, 10 mL) and saturated aqueous NaHCO₃ (10 mL), dried over Na₂SO₄, and evaporated under reduced pressure to give (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate.

LCMS: 99%, RT=2.35 min., (M+H)⁺=529 (method A).

### Synthesis of ((2R,4S,5S)-4,5-diazidotetrahydro-2H-pyran-2-yl)((S)-1-(4-fluorophenyl)-3,4-dihydroisoquinolin-2(1H)-yl)methanone

Under argon atmosphere, sodium azide (86 mg, 1.325 mmol) was added to a solution of (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate (200 mg, 0.378 mmol) in N,N-dimethylformamide (dry, 1.8 mL). The reaction mixture was warmed to 80° C. and stirred for 3 hours. After cooling to room temperature, the mixture was diluted with brine (10 mL) and extracted with ethyl acetate (3×15 mL). The combined organics were washed with brine (20 mL), dried on Na₂SO₄, and evaporated under reduced pressure. The residue was purified by flash column chromatography (silica, 0 to 40% ethyl acetate in heptane) to give ((2R,4S,5S)-4,5-diazidotetrahydro-2H-pyran-2-yl)((S)-1-(4-fluorophenyl)-3,4-dihydroisoquinolin-2(1H)-yl)methanone.

LCMS: 87%, RT=2.27 min., (M+H)⁺=422 (method A).

### Synthesis of ((2R,4S,5S)-4,5-diaminotetrahydro-2H-pyran-2-yl)((S)-1-(4-fluorophenyl)-3,4-dihydroisoquinolin-2(1H)-yl)methanone

A solution of ((2R,4S,5S)-4,5-diazidotetrahydro-2H-pyran-2-yl)((S)-1-(4-fluorophenyl)-3,4-dihydroisoquinolin-2(1H)-yl)methanone (0.164 g, 0.389 mmol) in tetrahydrofuran (3 mL) was hydrogenated in the presence of palladium (10 wt % on carbon, containing 50% water, 0.083 g, 0.039 mmol) at atmospheric hydrogen pressure. After 1.5 hours, the mixture was filtered over a 0.22 μm nylon filter and washed with ethyl acetate. The combined filtrates were concentrated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure to give ((2R,4S,5S)-4,5-diaminotetrahydro-2H-pyran-2-yl)((S)-1-(4-fluorophenyl)-3,4-dihydroisoquinolin-2(1H)-yl)methanone.

LCMS: 88%, RT=1.45 min., (M+H)⁺=370 (method A).

### Synthesis of ((S)-1-(4-fluorophenyl)-3,4-dihydroisoquinolin-2(1H)-yl)((3aS,6R,7aS)-1,3a,4,6,7,7a-hexahydropyrano[3,4-d]imidazol-6-yl)methanone

Trimethyl orthoformate (0.030 mL, 0.270 mmol) was added to a solution of ((2R,4S,5S)-4,5-diaminotetrahydro-2H-pyran-2-yl)((S)-1-(4-fluorophenyl)-3,4-dihydroisoquinolin-2(1H)-yl)methanone (90 mg, 0.244 mmol) in hexafluoroisopropanol (1.975 mL, 18.76 mmol). After 1 hour, the reaction mixture was concentrated under reduced and the residue was purified by acidic preparative MPLC (Linear Gradient: t=0 min 5% A, t=1 min 5% A; t=16 min 60% A; t=17 min 100%; t=22 min 100% A; detection: 220/254 nm). The product was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure. The product was further purified by preparative SFC (method AT) to give ((S)-1-(4-fluorophenyl)-3,4-dihydroisoquinolin-2(1H)-yl)((3aS,6R,7aS)-1,3a,4,6,7,7a-hexahydropyrano[3,4-d]imidazol-6-yl)methanone (COMPOUND 5430) lyophilization from a mixture of acetonitrile and water (1:1, 4 mL).

LCMS: 98%, RT=1.01 min., (M+H)⁺=380 (method P).

### Example 118

### ((S)-1-(4-fluorophenyl)-3,4-dihydroisoquinolin-2(1H)-yl)((2R,4S,5R)-4-hydroxy-5-(methylamino)tetrahydro-2H-pyran-2-yl)methanone (COMPOUND 5431)

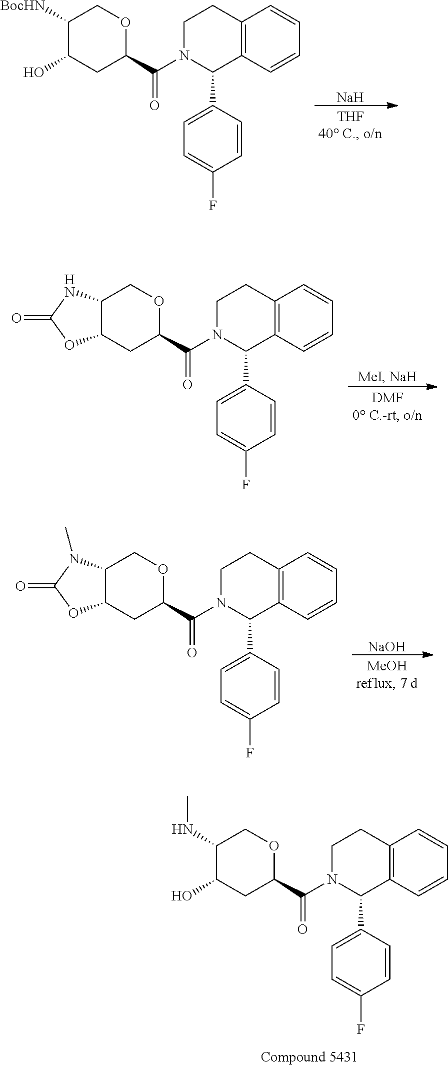

### Synthesis of (3aR,6R,7aS)-6-((S)-1-(4-fluorophenyl)-1,2,3,4-tetrahydroisoquinoline-2-carbonyl)tetrahydro-4H-pyrano[3,4-d]oxazol-2(3H)-one

Under an argon atmosphere, sodium hydride (60 wt % dispersion in mineral oil, 25.5 mg, 0.638 mmol) was added to a solution of tert-butyl ((3R,4S,6R)-6-((S)-1-(4-fluorophenyl)-1,2,3,4-tetrahydroisoquinoline-2-carbonyl)-4-hydroxytetrahydro-2H-pyran-3-yl)carbamate (see Compound 5052, 300 mg, 0.638 mmol) in tetrahydrofuran (dry, 15 mL). The reaction mixture was warmed to 40° C. and stirred overnight. Then, the mixture was cooled to room temperature and diluted with dichloromethane (15 mL) and saturated aqueous NH₄Cl (20 mL). The layers were separated and the aqueous phase was extracted with dichloromethane (2×15 mL). The combined organics were dried over Na₂SO₄ and evaporated under reduced pressure. The residue was purified by flash column chromatography (silica, 30 to 100% ethyl acetate in heptane) to give (3aR,6R,7aS)-6-((S)-1-(4-fluorophenyl)-1,2,3,4-tetrahydroisoquinoline-2-carbonyl)tetrahydro-4H-pyrano[3,4-d]oxazol-2(3H)-one.

LCMS: 99%, RT=2.07 min., (M+H)⁺=397 (method A).

### Synthesis of (3aR,6R,7aS)-6-((S)-1-(4-fluorophenyl)-1,2,3,4-tetrahydroisoquinoline-2-carbonyl)-3-methyltetrahydro-4H-pyrano[3,4-d]oxazol-2(3H)-one

Under a nitrogen atmosphere at 0° C., sodium hydride (60 wt % dispersion in mineral oil, 19.4 mg, 0.484 mmol) was added to a solution of (3aR,6R,7aS)-6-((S)-1-(4-fluorophenyl)-1,2,3,4-tetrahydroisoquinoline-2-carbonyl)tetrahydro-4H-pyrano[3,4-d]oxazol-2(3H)-one (0.16 g, 0.404 mmol) in N,N-dimethylformamide (dry, 4 mL). After 30 minutes, iodomethane (0.136 mL, 2.180 mmol) was added and the reaction mixture was allowed to slowly warm to room temperature. After stirring overnight, the mixture was diluted with a mixture of water and brine (1:1). The formed precipitate was collected by filtration, washed with water, and dried by air current on the filter to give (3aR,6R,7aS)-6-((S)-1-(4-fluorophenyl)-1,2,3,4-tetrahydroisoquinoline-2-carbonyl)-3-methyltetrahydro-4H-pyrano[3,4-d]oxazol-2(3H)-one.

LCMS: 100%, RT=1.95 min., (M+H)⁺=411 (method B).

### Synthesis of ((S)-1-(4-fluorophenyl)-3,4-dihydroisoquinolin-2(1H)-yl)((2R,4S,5R)-4-hydroxy-5-(methylamino)tetrahydro-2H-pyran-2-yl)methanone (COMPOUND 5431

Aqueous sodium hydroxide (2 M, 1.316 mL, 2.63 mmol) was added to a solution of (3aR,6R,7aS)-6-((S)-1-(4-fluorophenyl)-1,2,3,4-tetrahydroisoquinoline-2-carbonyl)-3-methyltetrahydro-4H-pyrano[3,4-d]oxazol-2(3H)-one (108 mg, 0.263 mmol) in methanol (15 mL) and the mixture was warmed to 70° C. After stirring overnight, additional sodium hydroxide (solid, 105 mg, 2.63 mmol) was added and stirring was continued for 5 days. Then, the mixture was warmed to 80° C. and stirred for another day. After cooling to room temperature, the solvent was partially removed under reduced pressure and the residue was diluted with water and aqueous HCl (1 M, 5 mL). The formed suspension was extracted with dichloromethane (twice) and the combined organic layers were dried over Na₂SO₄ and evaporated under reduced pressure. The residue was purified by preparative SFC (method BT) to give ((S)-1-(4-fluorophenyl)-3,4-dihydroisoquinolin-2(1H)-yl)((2R,4S,5R)-4-hydroxy-5-(methylamino)tetrahydro-2H-pyran-2-yl)methanone (COMPOUND 5431).

LCMS: 97%, RT=1.00 min., (M+H)⁺=385 (method P). SFC: 96%, RT=4.43 min., (M+H)⁺=385 (method F).

### Example 119

### ((2R,4S,5R)-4-amino-5-methyltetrahydro-2H-pyran-2-yl)((S)-1-(4-fluorophenyl)-3,4-dihydroisoquinolin-2(1H)-yl)methanone (COMPOUND 5518) and ((2R,4S,5S)-4-amino-5-methyltetrahydro-2H-pyran-2-yl)((S)-1-(4-fluorophenyl)-3,4-dihydroisoquinolin-2(1H)-yl)methanone (COMPOUND 5519)

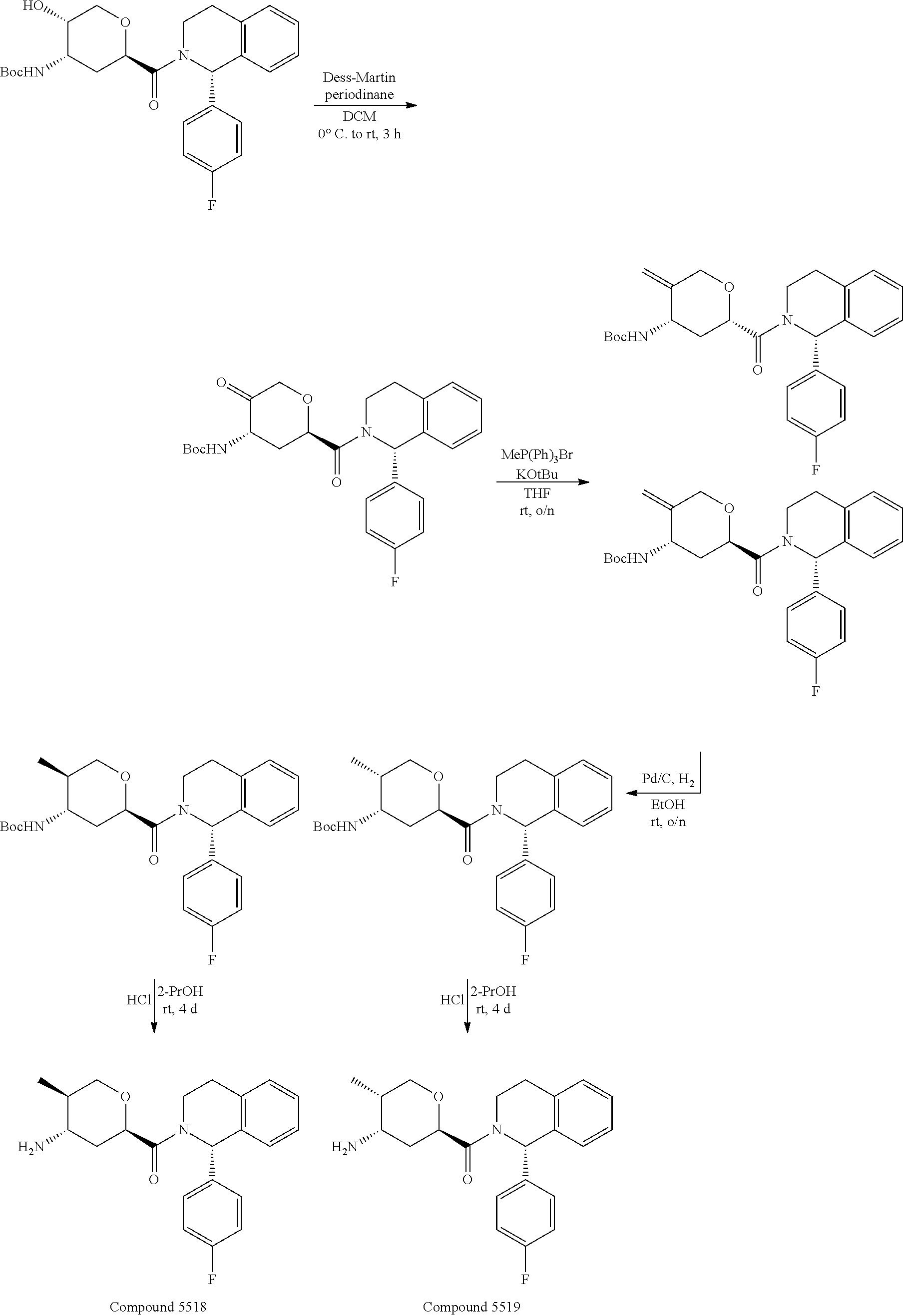

### Synthesis of tert-butyl ((2R,4S)-2-((S)-1-(4-fluorophenyl)-1,2,3,4-tetrahydroisoquinoline-2-carbonyl)-5-oxotetrahydro-2H-pyran-4-yl)carbamate

At 0° C., Dess-Martin periodinane (992 mg, 2.338 mmol) was added to a solution of tert-butyl ((2R,4S,5S)-2-((S)-1-(4-fluorophenyl)-1,2,3,4-tetrahydroisoquinoline-2-carbonyl)-5-hydroxytetrahydro-2H-pyran-4-yl)carbamate (see Compound 5052, 1000 mg, 2.125 mmol) in dichloromethane (20 mL). The resulting mixture was allowed to warm to room temperature. After 3 hours, the reaction mixture was diluted with a mixture of saturated aqueous Na₂S₂O₃ and saturated aqueous NaHCO₃ (1:1, 40 mL) and stirred for 30 minutes. The organic layer was separated and the aqueous layer was extracted with dichloromethane (2×40 mL). The combined organic layers were washed with brine (40 mL), dried over Na₂SO₄, and evaporated under reduced pressure. The residue was purified by flash column chromatography (silica, 0 to 50% ethyl acetate in heptane) to give tert-butyl ((2R,4S)-2-((S)-1-(4-fluorophenyl)-1,2,3,4-tetrahydroisoquinoline-2-carbonyl)-5-oxotetrahydro-2H-pyran-4-yl)carbamate.

LCMS: 100%, RT=2.02 min.+2.11-2.14 min., (M-(t-Bu)+H)⁺=413 (method A).

### Synthesis of tert-butyl ((2R,4S)-2-((S)-1-(4-fluorophenyl)-1,2,3,4-tetrahydroisoquinoline-2-carbonyl)-5-methylenetetrahydro-2H-pyran-4-yl)carbamate

At 0° C., potassium tert-butoxide (1.0 M in THF, 1.024 mL, 1.024 mmol) was added to a suspension of methyltriphenylphosphonium bromide (427 mg, 1.195 mmol) in tetrahydrofuran (dry, 5 mL). After 30 minutes, a solution of tert-butyl ((2R,4S)-2-((S)-1-(4-fluorophenyl)-1,2,3,4-tetrahydroisoquinoline-2-carbonyl)-5-oxotetrahydro-2H-pyran-4-yl)carbamate (400 mg, 0.854 mmol) in tetrahydrofuran (dry, 2.5 mL) was added dropwise. After the addition was complete, the reaction mixture was allowed to warm to room temperature and stirred overnight. The mixture was diluted with saturated aqueous ammonium chloride (5 mL) and dichloromethane (30 mL). The organic layer was separated, washed with brine (10 mL), dried over Na₂SO₄, and evaporated under reduced pressure. The residue was purified by flash column chromatography (silica, 0 to 50% ethyl acetate in heptane) and preparative SFC (method BR) to give tert-butyl ((2R,4S)-2-((S)-1-(4-fluorophenyl)-1,2,3,4-tetrahydroisoquinoline-2-carbonyl)-5-methylenetetrahydro-2H-pyran-4-yl)carbamate as the first eluting SFC isomer and tert-butyl ((2S,4S)-2-((S)-1-(4-fluorophenyl)-1,2,3,4-tetrahydroisoquinoline-2-carbonyl)-5-methylenetetrahydro-2H-pyran-4-yl)carbamate as the second eluting SFC isomer.

First eluting SFC isomer LCMS: 99%, RT=2.22 min., (M+H)⁺=467 (method A). SFC: 99%, RT=2.13 min., (M+H)⁺=467 (method V).

Second eluting SFC isomer LCMS: 92%, RT=2.19 min., (M+H)⁺=467 (method A). SFC: 97%, RT=2.36 min., (M+H)⁺=467 (method V).

### Synthesis of tert-butyl ((2R,4S,5R)-2-((S)-1-(4-fluorophenyl)-1,2,3,4-tetrahydroisoquinoline-2-carbonyl)-5-methyltetrahydro-2H-pyran-4-yl)carbamate

A solution of tert-butyl ((2R,4S)-2-((S)-1-(4-fluorophenyl)-1,2,3,4-tetrahydroisoquinoline-2-carbonyl)-5-methylenetetrahydro-2H-pyran-4-yl)carbamate (102 mg, 0.219 mmol) in ethanol (2 mL) was hydrogenated in the presence of palladium (10 wt % on carbon, 23.27 mg, 0.022 mmol) at atmospheric hydrogen pressure. After stirring overnight, the mixture was filtered and the filtrate was concentrated under reduced pressure. The residue was purified by preparative SFC (method BT) to give tert-butyl ((2R,4S,5R)-2-((S)-1-(4-fluorophenyl)-1,2,3,4-tetrahydroisoquinoline-2-carbonyl)-5-methyltetrahydro-2H-pyran-4-yl)carbamate as the first eluting SFC isomer and tert-butyl ((2R,4S,5S)-2-((S)-1-(4-fluorophenyl)-1,2,3,4-tetrahydroisoquinoline-2-carbonyl)-5-methyltetrahydro-2H-pyran-4-yl)carbamate as the second eluting SFC isomer.

First eluting SFC isomer. LCMS: 99%, RT=2.25 min., (M+H)⁺=469 (method A). SFC: 100%, RT=2.58 min., (M+H)⁺=469 (method F).

Second eluting SFC isomer. LCMS: 94%, RT=2.21 min., (M+H)⁺=469 (method A). SFC: 95%, RT=2.83 min., (M+H)⁺=469 (method F).

### Synthesis of ((2R,4S,5R)-4-amino-5-methyltetrahydro-2H-pyran-2-yl)((S)-1-(4-fluorophenyl)-3,4-dihydroisoquinolin-2(1H)-yl)methanone (COMPOUND 5518)

HCl (6 M in 2-propanol, 0.3 mL, 1.80 mmol) was added to a solution of tert-butyl ((2R,4S,5R)-2-((S)-1-(4-fluorophenyl)-1,2,3,4-tetrahydroisoquinoline-2-carbonyl)-5-methyltetrahydro-2H-pyran-4-yl)carbamate (59 mg, 0.126 mmol) in 2-propanol (3 mL). After 4 days, the reaction mixture was diluted with dichloromethane (10 mL) and saturated aqueous NaHCO₃ (10 mL). The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:2, 3 mL) to give ((2R,4S,5R)-4-amino-5-methyltetrahydro-2H-pyran-2-yl)((S)-1-(4-fluorophenyl)-3,4-dihydroisoquinolin-2(1H)-yl)methanone (COMPOUND 5518).

LCMS: 99%, RT=2.77 min., (M+H)⁺=369 (method AK).

### Synthesis of ((2R,4S,5S)-4-amino-5-methyltetrahydro-2H-pyran-2-yl)((S)-1-(4-fluorophenyl)-3,4-dihydroisoquinolin-2(1H)-yl)methanone (COMPOUND 5519)

HCl (6 M in 2-propanol, 0.1 mL, 0.60 mmol) was added to a solution of tert-butyl ((2R,4S,5S)-2-((S)-1-(4-fluorophenyl)-1,2,3,4-tetrahydroisoquinoline-2-carbonyl)-5-methyltetrahydro-2H-pyran-4-yl)carbamate (17 mg, 0.036 mmol) in 2-propanol (1 mL). After 4 days, the reaction mixture was diluted with dichloromethane (5 mL) and saturated aqueous NaHCO₃ (5 mL). The layers were separated using a phase-separator and the organic filtrate was evaporated under reduced pressure. The residue was dissolved in methanol (1 mL) and brought onto an SCX-2 column (1 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:2, 3 mL) to give ((2R,4S,5S)-4-amino-5-methyltetrahydro-2H-pyran-2-yl)((S)-1-(4-fluorophenyl)-3,4-dihydroisoquinolin-2(1H)-yl)methanone (COMPOUND 5519).

LCMS: 93%, RT=2.73 min., (M+H)⁺=369 (method AK).

### Example 120

### ((2R,4S,5R)-4-amino-5-(ethylthio)tetrahydro-2H-pyran-2-yl)((S)-1-(4-fluorophenyl)-3,4-dihydroisoquinolin-2(1H)-yl)methanone (COMPOUND 5521)

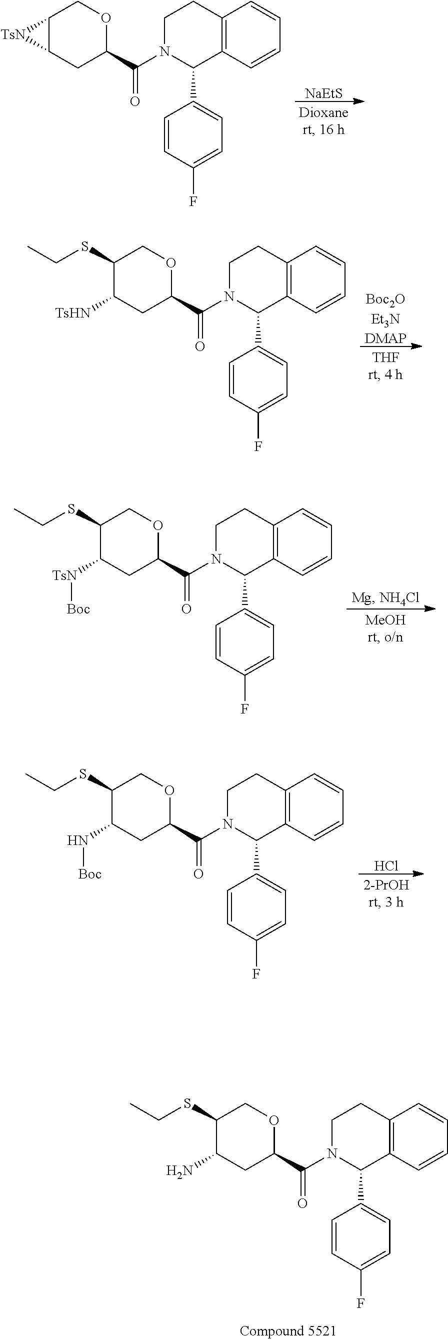

### Synthesis of N-((2R,4S,5R)-5-(ethylthio)-2-((S)-1-(4-fluorophenyl)-1,2,3,4-tetrahydroisoquinoline-2-carbonyl)tetrahydro-2H-pyran-4-yl)-4-methylbenzenesulfonamide

Sodium ethanethiolate (66.4 mg, 0.790 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 200 mg, 0.395 mmol) in 1,4-dioxane (6 mL). After stirring overnight, additional sodium ethanethiolate (33.2 mg, 0.395 mmol) was added and the mixture was warmed to 40° C.). After stirring overnight, additional sodium ethanethiolate (266 mg, 3.16 mmol) was added and stirring was continued for 3 more days. Then, the mixture was diluted with dichloromethane (10 mL), washed with saturated aqueous NaHCO₃, passed through a hydrophobic fritte, and evaporated under reduced pressure. The residue was purified by flash column chromatography (silica, 0 to 40% ethyl acetate in heptane) to give N-((2R,4S,5R)-5-(ethylthio)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 98%, RT=2.40 min., (M+H)⁺=569 (method A).

### Synthesis of tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.034 mL, 0.246 mmol), 4-dimethylaminopyridine (1.504 mg, 0.012 mmol), and di-tert-butyl dicarbonate (53.7 mg, 0.246 mmol) were added to a solution of N-((2R,4S,5R)-5-(ethylthio)-2-((S)-1-(4-fluorophenyl)-1,2,3,4-tetrahydroisoquinoline-2-carbonyl)tetrahydro-2H-pyran-4-yl)-4-methylbenzenesulfonamide (70 mg, 0.123 mmol) in tetrahydrofuran (5 mL). After stirring the reaction mixture for 3 hours, additional di-tert-butyl dicarbonate (26.9 mg, 0.123 mmol) was added and stirring was continued for another hour. Then, the mixture was concentrated under reduced pressure and the residue was purified by flash column chromatography (silica, 20 to 100% ethyl acetate in heptane) to give tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)(tosyl)carbamate.

LCMS: 98%, RT=2.43 min., (M+H)⁺=669 (method A).

### Synthesis of tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)carbamate

Magnesium (109 mg, 4.49 mmol) and ammonium chloride (48.0 mg, 0.897 mmol) were added to a solution of tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)(tosyl)carbamate (60 mg, 0.090 mmol) in methanol (5 mL). After stirring overnight, additional magnesium was added and the reaction was left to stir for an additional hour. Then, the mixture was diluted with saturated aqueous NH₄Cl and after stirring for a few minutes, the mixture was extracted with dichloromethane. The organic layer was passed through a phase separator and evaporated under reduced pressure. The residue was purified by flash column chromatography (silica, 0 to 100% ethyl acetate in heptane) to give tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)carbamate.

LCMS: 96%, RT=2.26 min., (M+H)⁺=515 (method A).

### Synthesis of ((2R,4S,5R)-4-amino-5-(ethylthio)tetrahydro-2H-pyran-2-yl)((S)-1-(4-fluorophenyl)-3,4-dihydroisoquinolin-2(1H)-yl)methanone (COMPOUND 5521)

HCl (6 M in 2-propanol, 2 mL, 12.00 mmol) was added to a solution of tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)carbamate (23 mg, 0.045 mmol) in 2-propanol (3 mL). After stirring for 2 hours, additional HCl (6 M in 2-propanol, 2 mL, 12.00 mmol) was added. After 1 hour, the reaction mixture was diluted with saturated aqueous NaHCO₃ and extracting with dichloromethane. The organic layer was passed through a phase separator and evaporated under reduced pressure. The residue was dissolved in methanol and brought onto an SCX-2 column and eluted with methanol until neutral. Next, the column was eluted with ammonia in methanol (2 M). The basic fraction was concentrated to dryness under reduced pressure. The residue was lyophilized from a mixture of acetonitrile and water to give ((2R,4S,5R)-4-amino-5-(ethylthio)tetrahydro-2H-pyran-2-yl)((S)-1-(4-fluorophenyl)-3,4-dihydroisoquinolin-2(1H)-yl)methanone (COMPOUND 5521).

LCMS: 97%, RT=1.18 min., (M+H)⁺=415 (method P).

### Example 121

### ((2R,4S,5R)-4-amino-5-(methylthio)tetrahydro-2H-pyran-2-yl)((S)-1-(4-fluorophenyl)-3,4-dihydroisoquinolin-2(1H)-yl)methanone (COMPOUND 5520)

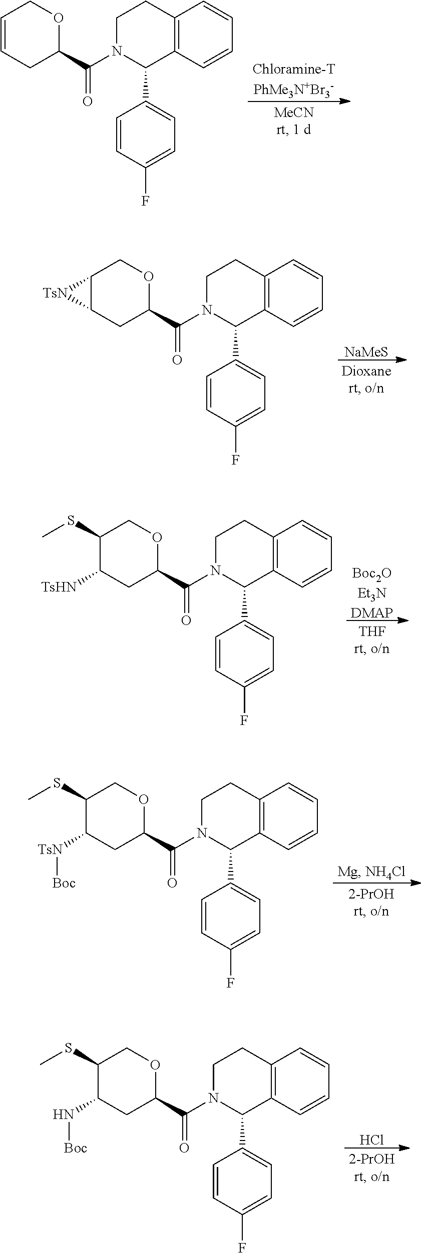

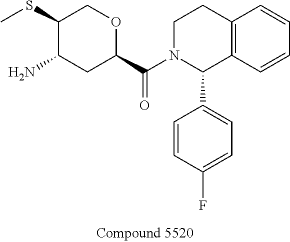 [SMILES: CS[C@H]1CO[C@@H](C(=O)N2CCc3ccccc3[C@@H]2c2ccc(F)cc2)C[C@@H]1N]

### Synthesis of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone

Chloramine-T hydrate (801 mg, 3.26 mmol) and phenyltrimethylammonium tribromide (111 mg, 0.296 mmol) were added to a solution of ((R)-3,6-dihydro-2H-pyran-2-yl)((S)-1-(4-fluorophenyl)-3,4-dihydroisoquinolin-2(1H)-yl)methanone (see Compound 5053, 1.0 g, 2.96 mmol) in acetonitrile (12.5 mL). After 1 day, the white precipitate was filtered off, washed with acetonitrile, and dried by air current on the filter to give ((S)-1-(4-fluorophenyl)-3,4-dihydroisoquinolin-2(1H)-yl)((1 S,4R,6S)-7-tosyl-3-oxa-7-azabicyclo[4.1.0]heptan-4-yl)methanone.

LCMS: 96%, RT=2.18 min., (M+H)⁺=507 (method A).

### Synthesis of N-((2R,4S,5R)-2-((S)-1-(4-fluorophenyl)-1,2,3,4-tetrahydroisoquinoline-2-carbonyl)-5-(methylthio)tetrahydro-2H-pyran-4-yl)-4-methylbenzenesulfonamide

Sodium methanethiolate (55.3 mg, 0.790 mmol) was added to a solution of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (200 mg, 0.395 mmol) in 1,4-dioxane (6 mL). After stirring overnight, the mixture was diluted with dichloromethane (10 mL), washed with saturated aqueous NaHCO₃, passed through a hydrophobic fritte, and evaporated under reduced pressure. The residue was purified by flash column chromatography (silica, 0 to 40% ethyl acetate in heptane) to give N-((2R,4S,5R)-2-((S)-1-(4-fluorophenyl)-1,2,3,4-tetrahydroisoquinoline-2-carbonyl)-5-(methylthio)tetrahydro-2H-pyran-4-yl)-4-methylbenzenesulfonamide.

LCMS: 99%, RT=2.20 min., (M+H)⁺=555 (method A).

### Synthesis of tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)(tosyl)carbamate

Triethylamine (0.035 mL, 0.252 mmol), 4-dimethylaminopyridine (1.5 mg, 0.013 mmol), and di-tert-butyl dicarbonate (55.1 mg, 0.252 mmol) were added to a solution of N-((2R,4S,5R)-2-((S)-1-(4-fluorophenyl)-1,2,3,4-tetrahydroisoquinoline-2-carbonyl)-5-(methylthio)tetrahydro-2H-pyran-4-yl)-4-methylbenzenesulfonamide (70 mg, 0.126 mmol) in tetrahydrofuran (5 mL). After 1 hour, the mixture was concentrated under reduced pressure. The residue was purified by flash column chromatography (silica, 20 to 100% ethyl acetate in heptane) to give tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)(tosyl)carbamate.

LCMS: 99%, RT=2.40 min., (M+H)⁺=655 (method A).

### Synthesis of tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)carbamate

Magnesium (120 mg, 4.94 mmol) and ammonium chloride (65.3 mg, 1.22 mmol) were added to a solution of tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)(tosyl)carbamate (80 mg, 0.122 mmol) in methanol (5 mL). After stirring overnight, additional magnesium (50 mg, 1.24 mmol) and ammonium chloride (20 mg, 0.37 mmol) were added and stirring was continued for 2 hours. Then, the mixture was diluted saturated aqueous NH₄Cl, stirred for a few minutes, and extracted with dichloromethane. The combined organics were passed through a phase separator and evaporated under reduced pressure. The residue was purified by flash column chromatography (silica, 0 to 100% ethyl acetate in heptane) to give tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)carbamate.

LCMS: 94%, RT=2.21 min., (M+H)⁺=501 (method A).

### Synthesis of ((2R,4S,5R)-4-amino-5-(methylthio)tetrahydro-2H-pyran-2-yl)((S)-1-(4-fluorophenyl)-3,4-dihydroisoquinolin-2(1H)-yl)methanone (COMPOUND 5520)

HCl (6 M in 2-propanol, 3 mL, 18.0 mmol) was added to a solution of tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)carbamate (36 mg, 0.072 mmol) in 2-propanol (3 mL). After 2 hours, additional HCl (6 M in 2-propanol, 2 mL, 12.0 mmol) was added and stirring was continued overnight. The reaction mixture was diluted with dichloromethane and saturated aqueous NaHCO₃. The layers were separated using a phase-separator and the organic filtrate was evaporated under reduced pressure. The residue was dissolved in methanol and brought onto an SCX-2 column and eluted with methanol until neutral. Next, the column was eluted with ammonia in methanol (2 M). The basic fraction was concentrated to dryness under reduced pressure. The residue was lyophilized from a mixture of acetonitrile and water to give ((2R,4S,5R)-4-amino-5-(methylthio)tetrahydro-2H-pyran-2-yl)((S)-1-(4-fluorophenyl)-3,4-dihydroisoquinolin-2(1H)-yl)methanone (COMPOUND 5520).

LCMS: 97%, RT=1.13 min., (M+H)⁺=401 (method P).

### Example 122

### ((2R,4S,5R)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5523)

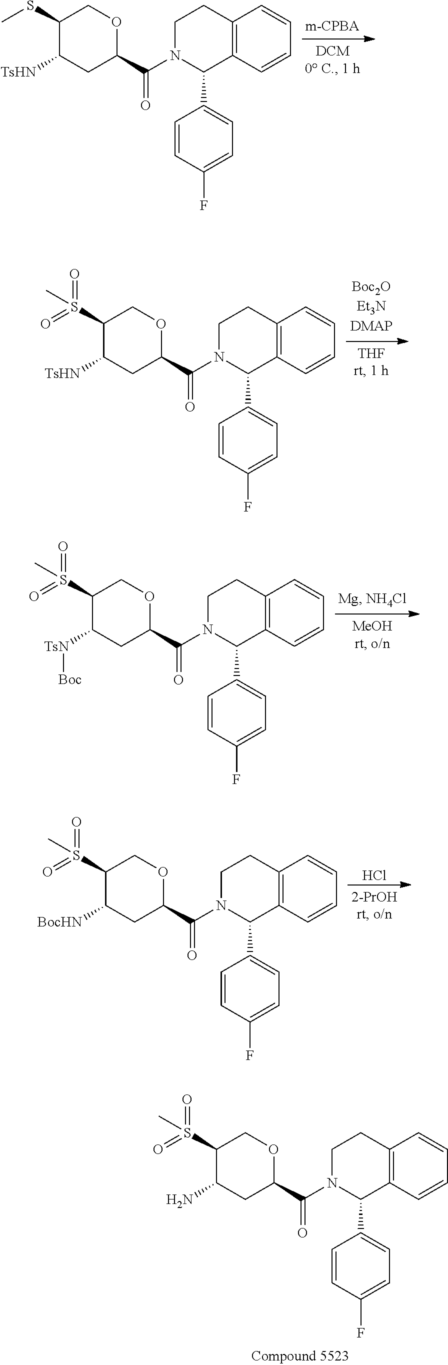

### Synthesis of N-((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)-4-methylbenzenesulfonamide

At 0° C., m-chloroperbenzoic acid (70 wt %, 78 mg, 0.315 mmol) was added to a solution of N-((2R,4S,5R)-2-((S)-1-(4-fluorophenyl)-1,2,3,4-tetrahydroisoquinoline-2-carbonyl)-5-(methylthio)tetrahydro-2H-pyran-4-yl)-4-methylbenzenesulfonamide (see COMPOUND 5520, 70 mg, 0.126 mmol) in dichloromethane (4 mL). After 1 hour, the reaction mixture was diluted with a mixture of saturated aqueous NaHCO₃, aqueous NaOH (1 M), and water (1:1:1). The organic layer was separated, using a phase separator, and evaporated under reduced pressure to give N-((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)-4-methylbenzenesulfonamide.

LCMS: 93%, RT=2.07 min., (M+H)⁺=587 (method A).

### Synthesis of tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)(tosyl)carbamate

Triethylamine (0.033 mL, 0.239 mmol), 4-dimethylaminopyridine (1.458 mg, 0.012 mmol) and di-tert-butyl dicarbonate (52.1 mg, 0.239 mmol) were added to a solution of N-((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)-4-methylbenzenesulfonamide (70 mg, 0.119 mmol) in tetrahydrofuran (5 mL). After 1 hour, the mixture was concentrated under reduced pressure. The residue was purified by flash column chromatography (silica, 20 to 100% ethyl acetate in heptane) to give tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)(tosyl)carbamate.

LCMS: 96%, RT=2.28 min., (M+H)⁺=687 (method A).

### Synthesis of tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)carbamate

Magnesium (133 mg, 5.46 mmol) and ammonium chloride (58.4 mg, 1.092 mmol) were added to a solution of tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)(tosyl)carbamate (75 mg, 0.109 mmol) in methanol (5 mL). After stirring overnight, the mixture was diluted with saturated aqueous NH₄Cl and the reaction was stirred for a few minutes. Then, the mixture was extracted with dichloromethane. The combined organics were passed through a phase separator and evaporated under reduced pressure. The residue was purified by flash column chromatography (silica, 0 to 100% ethyl acetate in heptane) to give tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)carbamate.

LCMS: 99%, RT=2.08 min., (M+H)⁺=533 (method A).

### Synthesis of ((2R,4S,5R)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5523)

HCl (6 M in 2-propanol, 2 mL, 12.0 mmol) was added to a solution of tert-butyl ((2R,4S,5R)-2-((S)-1-(4-fluorophenyl)-1,2,3,4-tetrahydroisoquinoline-2-carbonyl)-5-(methylsulfonyl)tetrahydro-2H-pyran-4-yl)carbamate (45 mg, 0.084 mmol) in 2-propanol (3 mL). After 2 hours, additional HCl (6 M in 2-propanol, 2 mL, 12.0 mmol) was added and the mixture was left to stir overnight after which another portion of HCl (6 M in 2-propanol, 2 mL, 12.0 mmol) was added. After 3 hours, the mixture was basified with NaHCO₃ and extracted with dichloromethane. The combined organics were passed through a phase separator and evaporated under reduced pressure. The residue was dissolved in methanol and brought onto an SCX-2 column (1 g) and eluted with methanol until neutral. Next, the column was eluted with ammonia in methanol (2 M). The basic fraction was concentrated to dryness under reduced pressure. The residue was purified by acidic preparative MPLC (Linear Gradient: t=0 min 10% A; t=1 min 10% A; t=16 min 50% A; t=17 min 100%; t=22 min 100% A; detection: 220 nm). The product containing fraction were combined, basified with saturated aqueous NaHCO₃, and the volatiles were removed under reduced pressure. The aqueous residue was extracted with dichloromethane and the combined organics were passed through a phase separator and evaporated under reduced pressure. The residue was lyophilised from a mixture of acetonitrile and water to give ((2R,4S,5R)-4-amino-5-(methylsulfonyl)tetrahydro-2H-pyran-2-yl)((S)-1-(4-fluorophenyl)-3,4-dihydroisoquinolin-2(1H)-yl)methanone (COMPOUND 5523).

LCMS: 99%, RT=1.06 min., (M+H)⁺=433 (method P).

### Example 123

### ((2R,4S,5R)-4-amino-5-(ethylsulfonyl)tetrahydro-2H-pyran-2-yl)((S)-1-(4-fluorophenyl)-3,4-dihydroisoquinolin-2(1H)-yl)methanone (COMPOUND 5524)

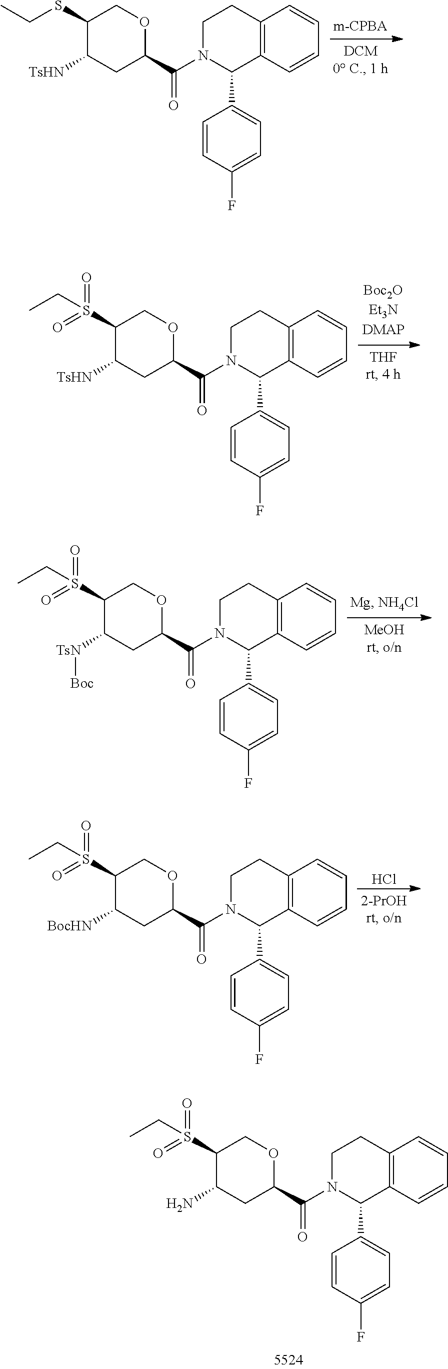

### Synthesis of N-((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)-4-methylbenzenesulfonamide

At 0° C., m-chloroperbenzoic acid (70 wt %, 76 mg, 0.308 mmol) was added to a solution of N-((2R,4S,5R)-5-(ethylthio)-2-((S)-1-(4-fluorophenyl)-1,2,3,4-tetrahydroisoquinoline-2-carbonyl)tetrahydro-2H-pyran-4-yl)-4-methylbenzenesulfonamide (see Compound 5521, 70 mg, 0.123 mmol) in dichloromethane (4 mL). After 1 hour, the reaction mixture was diluted with a mixture of saturated aqueous NaHCO₃, aqueous NaOH (1 M), and water (1:1:1). The organic layer was separated, using a phase separator, and evaporated under reduced pressure to give N-((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 96%, RT=2.10 min., (M+H)⁺=601 (method A).

### Synthesis of tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.032 ml, 0.233 mmol), 4-dimethylaminopyridine (1.424 mg, 0.012 mmol) and di-tert-butyl dicarbonate (50.9 mg, 0.233 mmol) were added to a solution of N-((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)-4-methylbenzenesulfonamide (70 mg, 0.117 mmol) in tetrahydrofuran (5 mL). After respectively 2 and 3 hours, additional portions of di-tert-butyl dicarbonate (50.9 mg, 0.233 mmol and 25.4 mg, 0.117 mmol) were added and stirring was continued for another hour. The mixture was concentrated under reduced pressure and the residue was purified by flash column chromatography (silica, 20 to 100% ethyl acetate in heptane) to give tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)(tosyl)carbamate.

LCMS: 94%, RT=2.31 min., (M+H)⁺=701 (method A).

### Synthesis of tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)carbamate

Magnesium (120 mg, 4.92 mmol) and ammonium chloride (52.7 mg, 0.985 mmol) were added to a solution of tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)(tosyl)carbamate (69 mg, 0.098 mmol) in methanol (5 mL). After stirring overnight, the mixture was diluted with saturated aqueous NH₄Cl and the reaction was stirred for a few minutes. Then, the mixture was extracted with dichloromethane. The combined organics were passed through a phase separator and evaporated under reduced pressure. The residue was purified by flash column chromatography (silica, 0 to 100% ethyl acetate in heptane) to give tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)carbamate.

LCMS: 100%, RT=2.12 min., (M+H)⁺=547 (method A).

### Synthesis of ((2R,4S,5R)-4-amino-5-(ethylsulfonyl)tetrahydro-2H-pyran-2-yl)((S)-1-(4-fluorophenyl)-3,4-dihydroisoquinolin-2(1H)-yl)methanone (COMPOUND 5524)

HCl (6 M in 2-propanol, 2 mL, 12.0 mmol) was added to a solution of tert-butyl ((2R,4S,5R)-5-(ethylsulfonyl)-2-((S)-1-(4-fluorophenyl)-1,2,3,4-tetrahydroisoquinoline-2-carbonyl)tetrahydro-2H-pyran-4-yl)carbamate (39 mg, 0.071 mmol) in 2-propanol (3 mL). After 5 hours, additional HCl (6 M in 2-propanol, 2 mL, 12.0 mmol) was added and the mixture was left to stir overnight after which another portion of HCl (6 M in 2-propanol, 2 mL, 12.0 mmol) was added. After 3 hours, the mixture was basified with NaHCO₃ and extracted with dichloromethane. The combined organics were passed through a phase separator and evaporated under reduced pressure. The residue was dissolved in methanol and brought onto an SCX-2 column (1 g) and eluted with methanol until neutral. Next, the column was eluted with ammonia in methanol (2 M). The basic fraction was concentrated to dryness under reduced pressure. The residue was purified by acidic preparative MPLC (Linear Gradient: t=0 min 10% A; t=1 min 10% A; t=16 min 50% A; t=17 min 100%; t=22 min 100% A; detection: 220 nm). The product containing fraction were combined, basified with saturated aqueous NaHCO₃, and the volatiles were removed under reduced pressure. The aqueous residue was extracted with dichloromethane and the combined organics were passed through a phase separator and evaporated under reduced pressure. The residue was lyophilized from a mixture of acetonitrile and water to give ((2R,4S,5R)-4-amino-5-(ethylsulfonyl)tetrahydro-2H-pyran-2-yl)((S)-1-(4-fluorophenyl)-3,4-dihydroisoquinolin-2(1H)-yl)methanone (COMPOUND 5524).

LCMS: 99%, RT=1.09 min., (M+H)⁺=447 (method P).

### Example 124

### ((S)-1-(4-fluorophenyl)-3,4-dihydroisoquinolin-2(1H)-yl)((4aR,7R,8aS)-hexahydro-2H,5H-pyrano[4,3-b][1,4]oxazin-7-yl)methanone (COMPOUND 5432)

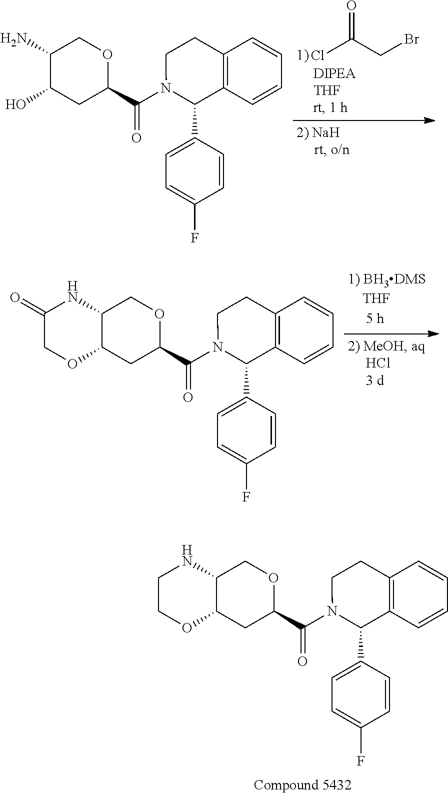

### Synthesis of (4aR,7R,8aS)-7-((S)-1-(4-fluorophenyl)-1,2,3,4-tetrahydroisoquinoline-2-carbonyl)tetrahydro-2H,5H-pyrano[4,3-b][1,4]oxazin-3(4H)-one

A solution of bromoacetyl chloride (0.036 mL, 0.431 mmol) in tetrahydrofuran (3.5 mL) was added to a solution of ((2R,4S,5R)-5-amino-4-hydroxytetrahydro-2H-pyran-2-yl)((S)-1-(4-fluorophenyl)-3,4-dihydroisoquinolin-2(1H)-yl)methanone (see Compound 5052, 145 mg, 0.391 mmol) and N,N-diisopropylethylamine (0.205 mL, 1.174 mmol) in tetrahydrofuran (3.5 mL). After 1 hour, sodium hydride (60 wt % dispersion in mineral oil, 47.0 mg, 1.174 mmol) was added. After stirring overnight, the reaction mixture was quenched with saturated aqueous ammonium chloride (15 mL) and diluted with dichloromethane (10 mL). The layers were separated and the aqueous layer was extracted with dichloromethane (2×10 mL). The combined organics were passed through a phase separator and evaporated under reduced pressure. The residue was suspended in a mixture of methanol and acetonitril (˜1:1, 5 mL) and filtered. The filtrate was purified by acidic preparative MPLC (Linear Gradient: t=0 min 5% A; t=1 min 5% A; t=2 min 20% A; t=17 min 60% A; t=18 min 100%; t=23 min 100% A; detection: 220 nm) to give (4aR,7R,8aS)-7-((S)-1-(4-fluorophenyl)-1,2,3,4-tetrahydroisoquinoline-2-carbonyl)tetrahydro-2H,5H-pyrano[4,3-b][1,4]oxazin-3(4H)-one.

LCMS: 95%, RT=1.86 min., (M+H)⁺=411 (method A).

### Synthesis of ((S)-1-(4-fluorophenyl)-3,4-dihydroisoquinolin-2(1H)-yl)((4aR,7R,8aS)-hexahydro-2H,5H-pyrano[4,3-b][1,4]oxazin-7-yl)methanone (COMPOUND 5432)

Borane-methyl sulfide complex (2 M, 0.034 mL, 0.068 mmol) was added to a solution of (4aR,7R,8aS)-7-((S)-1-(4-fluorophenyl)-1,2,3,4-tetrahydroisoquinoline-2-carbonyl)tetrahydro-2H,5H-pyrano[4,3-b][1,4]oxazin-3(4H)-one (7 mg, 0.017 mmol) tetrahydrofuran (dry, 1 mL). After 3 hours, another portion of borane-methyl sulfide complex (2 M, 0.017 mL, 0.034 mmol) was added and stirring was continued for 2 hours. Then, a mixture of aqueous HCl (1 M) and methanol (1:1, 1 mL) was added the reaction mixture was stirred for 3 days after which it was diluted with dichloromethane (5 mL) and saturated aqueous NaHCO₃ (5 mL). The layers were separated using a phase-separator and the organic filtrate was evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product was taken up in methanol and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give ((S)-1-(4-fluorophenyl)-3,4-dihydroisoquinolin-2(1H)-yl)((4aR,7R,8aS)-hexahydro-2H,5H-pyrano[4,3-b][1,4]oxazin-7-yl)methanone (COMPOUND 5432).

LCMS: 99%, RT=1.05 min., (M+H)⁺=397 (method P).

### Example 125

### ((2R,4S,5R)-4-amino-5-(benzyloxy)tetrahydro-2H-pyran-2-yl)((S)-1-(4-fluorophenyl)-3,4-dihydroisoquinolin-2(1H)-yl)methanone (COMPOUND 5531)

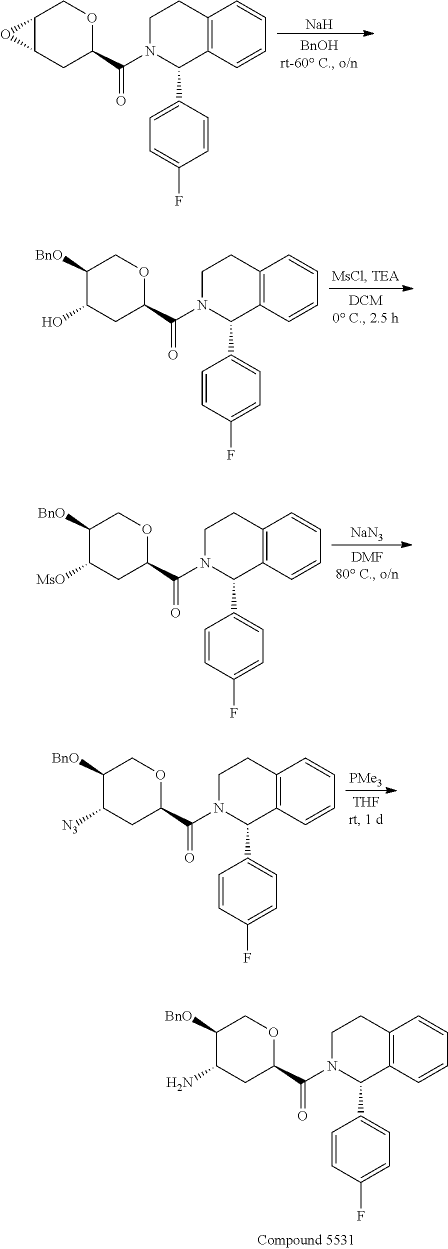

### Synthesis of ((2R,4S,5S)-5-(benzyloxy)-4-hydroxytetrahydro-2H-pyran-2-yl)((S)-1-(4-fluorophenyl)-3,4-dihydroisoquinolin-2(1H)-yl)methanone

Sodium hydride (60 wt % dispersion in mineral oil, 0.050 g, 1.245 mmol) was added to benzyl alcohol (1 mL, 9.66 mmol). After 5 minutes, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 0.20 g, 0.566 mmol) was added and the reaction mixture was stirred at room temperature overnight. The mixture was diluted with dichloromethane (10 mL) and washed with saturated aqueous NH₄Cl (5 mL). The aqueous layer was extracted with dichloromethane (2×5 mL) and the combined organic layers were passed through a phase separator and evaporated under reduced pressure. The residue was purified by flash column chromatography (silica, 30 to 70% ethyl acetate in heptane) to give ((2R,4S,5S)-5-(benzyloxy)-4-hydroxytetrahydro-2H-pyran-2-yl)((S)-1-(4-fluorophenyl)-3,4-dihydroisoquinolin-2(1H)-yl)methanone.

LCMS: 98%, RT=2.10 min., (M+H)⁺=462 (method A).

### Synthesis of (2R,4S,5S)-5-(benzyloxy)-2-((S)-1-(4-fluorophenyl)-1,2,3,4-tetrahydroisoquinoline-2-carbonyl)tetrahydro-2H-pyran-4-yl methanesulfonate

At 0° C., triethylamine (0.136 mL, 0.975 mmol) followed by methanesulfonyl chloride (0.038 mL, 0.488 mmol) were added to a solution of ((2R,4S,5S)-5-(benzyloxy)-4-hydroxytetrahydro-2H-pyran-2-yl)((S)-1-(4-fluorophenyl)-3,4-dihydroisoquinolin-2(1H)-yl)methanone (150 mg, 0.325 mmol) in dichloromethane (10 mL). After 2 hours, another portion of triethylamine (0.091 mL, 0.650 mmol) followed by methanesulfonyl chloride (0.025 mL, 0.325 mmol) were added. After 0.5 h, the mixture was diluted with saturated aqueous NaHCO₃ and the layers were separated. The organic layer was washed with saturated aqueous NH₄Cl, passed through a phase separator, and evaporated under reduces pressure to give (2R,4S,5S)-5-(benzyloxy)-2-((S)-1-(4-fluorophenyl)-1,2,3,4-tetrahydroisoquinoline-2-carbonyl)tetrahydro-2H-pyran-4-yl methanesulfonate (174 mg) as a white solid.

LCMS: 97%, RT=2.20 min., (M+H)⁺=540 (method A).

### Synthesis of ((2R,4S,5R)-4-azido-5-(benzyloxy)tetrahydro-2H-pyran-2-yl)((S)-1-(4-fluorophenyl)-3,4-dihydroisoquinolin-2(1H)-yl)methanone

Sodium azide (62.5 mg, 0.962 mmol) was added to a solution of (2R,4S,5S)-5-(benzyloxy)-2-((S)-1-(4-fluorophenyl)-1,2,3,4-tetrahydroisoquinoline-2-carbonyl)tetrahydro-2H-pyran-4-yl methanesulfonate (173 mg, 0.321 mmol) in N,N-dimethylformamide (4 mL) after which the reaction mixture was warmed to 80° C. After stirring overnight, the mixture was cooled to room temperature and partitioned between water and ethyl acetate. The organic layer was dried on Na₂SO₄ and evaporated under reduced pressure. The residue was purified by flash column chromatography (silica, 0 to 100% ethyl acetate in heptane) to give ((2R,4S,5R)-4-azido-5-(benzyloxy)tetrahydro-2H-pyran-2-yl)((S)-1-(4-fluorophenyl)-3,4-dihydroisoquinolin-2(1H)-yl)methanone.

LCMS: 99%, RT=2.31 min., (M+H)⁺=487 (method A).

### Synthesis of ((2R,4S,5R)-4-amino-5-(benzyloxy)tetrahydro-2H-pyran-2-yl)((S)-1-(4-fluorophenyl)-3,4-dihydroisoquinolin-2(1H)-yl)methanone (COMPOUND 5531)

Trimethylphosphine (1.0 M in tetrahydrofuran, 370 μl, 0.370 mmol) was added to a solution of ((2R,4S,5R)-4-azido-5-(benzyloxy)tetrahydro-2H-pyran-2-yl)((S)-1-(4-fluorophenyl)-3,4-dihydroisoquinolin-2(1H)-yl)methanone (120 mg, 0.247 mmol) in tetrahydrofuran. After stirring overnight, an additional portion of trimethylphosphine (1.0 M in tetrahydrofuran, 247 μl, 0.247 mmol) was added and the reaction was stirred for another 4 hours. Then, the reaction mixture was diluted with aqueous NaOH (1 M) and stirred for 5 minutes. The mixture was extracted with dichloromethane and the combined organics were passed through a phase separator and evaporated under reduced pressure. The residue was dissolved in methanol, brought onto an SCX-2 column, and eluted with methanol (5 column volumes). Next, the column was eluted with ammonia in methanol (2 M). The basic fraction was concentrated to dryness under reduced pressure. The residue was lyophilized from a mixture of acetonitrile and water to give ((2R,4S,5R)-4-amino-5-(benzyloxy)tetrahydro-2H-pyran-2-yl)((S)-1-(4-fluorophenyl)-3,4-dihydroisoquinolin-2(1H)-yl)methanone (COMPOUND 5531).

LCMS: 99%, RT=1.27 min., (M+H)⁺=461 (method P).

### Example 126

### ((2R,4S,5R)-4-amino-5-cyclopropoxytetrahydro-2H-pyran-2-yl)((S)-1-(4-fluorophenyl)-3,4-

### dihydroisoquinolin-2(1H)-yl)methanone (COMPOUND 5530)

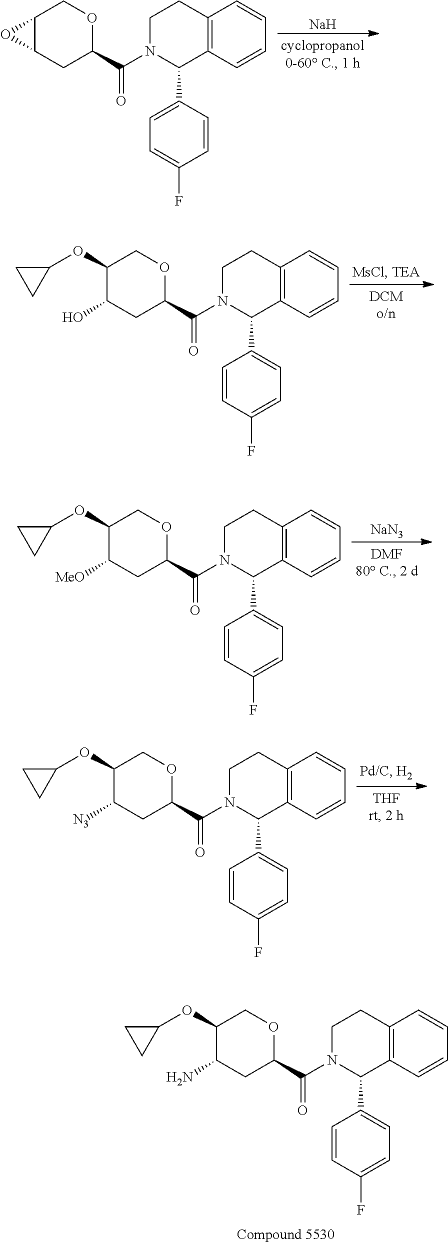

### Synthesis of ((2R,4S,5S)-5-cyclopropoxy-4-hydroxytetrahydro-2H-pyran-2-yl)((S)-1-(4-fluorophenyl)-3,4-dihydroisoquinolin-2(1H)-yl)methanone

At 0° C., sodium hydride (60 wt % dispersion in mineral oil, 32.2 mg, 0.805 mmol) was added portionwise to cyclopropanol (574 μL, 9.07 mmol). After 5 minutes, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 115 mg, 0.325 mmol) was added and the reaction mixture was warmed to 60° C. After 1 hour, the mixture was cooled to room temperature, diluted with dichloromethane (3 mL), and washed with saturated aqueous NH₄Cl (3 mL). The organic layer was passed through a phase separator and evaporated under reduced pressure. The residue was purified by flash column chromatography (silica, 0 to 100% ethyl acetate in heptane) to give ((2R,4S,5S)-5-cyclopropoxy-4-hydroxytetrahydro-2H-pyran-2-yl)((S)-1-(4-fluorophenyl)-3,4-dihydroisoquinolin-2(1H)-yl)methanone (impure, 99 mg) as a colorless oil that was used as such.

LCMS: 93%, RT=1.97 min., (M+H)⁺=412 (method A).

### Synthesis of (2R,4S,5S)-5-cyclopropoxy-2-((S)-1-(4-fluorophenyl)-1,2,3,4-tetrahydroisoquinoline-2-carbonyl)tetrahydro-2H-pyran-4-yl methanesulfonate

Triethylamine (0.099 mL, 0.715 mmol) followed by methanesulfonyl chloride (0.028 mL, 0.357 mmol) were added to a solution of ((2R,4S,5S)-5-cyclopropoxy-4-hydroxytetrahydro-2H-pyran-2-yl)((S)-1-(4-fluorophenyl)-3,4-dihydroisoquinolin-2(1H)-yl)methanone (impure, 98 mg, 0.238 mmol) in dichloromethane (4 mL). After 1 hour, another portion of methanesulfonyl chloride (9.22 μL, 0.119 mmol) was added and stirring was continued overnight. The reaction mixture was washed with aqueous citric acid (0.5 M, 4 mL) and saturated aqueous NaHCO₃ (4 mL). The organic layer was passed through a phase separator and evaporated under reduced pressure to give (2R,4S,5S)-5-cyclopropoxy-2-((S)-1-(4-fluorophenyl)-1,2,3,4-tetrahydroisoquinoline-2-carbonyl)tetrahydro-2H-pyran-4-yl methanesulfonate (impure, 101 mg) as a yellow foam/oil that was used as such.

LCMS: 91%, RT=2.16 min., (M+H)⁺=490 (method B).

### Synthesis of ((2R,4S,5R)-4-azido-5-cyclopropoxytetrahydro-2H-pyran-2-yl)((S)-1-(4-fluorophenyl)-3,4-dihydroisoquinolin-2(1H)-yl)methanone

Sodium azide (40.2 mg, 0.619 mmol) was added to a solution of (2R,4S,5S)-5-cyclopropoxy-2-((S)-1-(4-fluorophenyl)-1,2,3,4-tetrahydroisoquinoline-2-carbonyl)tetrahydro-2H-pyran-4-yl methanesulfonate (101 mg, 0.206 mmol) in N,N-dimethylformamide (4 mL) after which the reaction mixture was warmed to 80° C. After stirring overnight, an additional portion of sodium azide (40.2 mg, 0.619 mmol) was added and stirring was continued overnight. Then, the mixture was cooled to room temperature and partitioned between water and ethyl acetate. The organic layer was dried over Na₂SO₄ and evaporated under reduced pressure. The residue was purified by flash column chromatography (silica, 0 to 100% ethyl acetate in heptane) to give ((2R,4S,5R)-4-azido-5-cyclopropoxytetrahydro-2H-pyran-2-yl)((S)-1-(4-fluorophenyl)-3,4-dihydroisoquinolin-2(1H)-yl)methanone.

LCMS: 96%, RT=2.24 min., (M+H)⁺=437 (method A).

### Synthesis of ((2R,4S,5R)-4-amino-5-cyclopropoxytetrahydro-2H-pyran-2-yl)((S)-1-(4-fluorophenyl)-3,4-dihydroisoquinolin-2(1H)-yl)methanone (COMPOUND 5530)

A solution of ((2R,4S,5R)-4-azido-5-cyclopropoxytetrahydro-2H-pyran-2-yl)((S)-1-(4-fluorophenyl)-3,4-dihydroisoquinolin-2(1H)-yl)methanone (40 mg, 0.092 mmol) in tetrahydrofuran (4 mL) was hydrogenated in the presence of palladium (10 wt % on carbon, containing 50% water, 19.50 mg, 9.16 μmol) at atmospheric hydrogen pressure. After stirring for 2 hours, the mixture was filtered over a 0.22 μm nylon filter. The filtrate was concentrated under reduced pressure. The residue was dissolved in methanol, loaded onto an SCX-2 column (1 g), and eluted with methanol (5 column volumes). Next, the column was eluted with ammonia in methanol (2 M).

The basic fraction was concentrated to dryness under reduced pressure to give ((2R,4S,5R)-4-amino-5-cyclopropoxytetrahydro-2H-pyran-2-yl)((S)-1-(4-fluorophenyl)-3,4-dihydroisoquinolin-2(1H)-yl)methanone (COMPOUND 5530).

LCMS: 97%, RT=1.15 min., (M+H)⁺=411 (method P).

### Example 127

### ((2R,4S,5R)-4-amino-5-phenoxytetrahydro-2H-pyran-2-yl)((S)-1-(4-fluorophenyl)-3,4-dihydroisoquinolin-2(1H)-yl)methanone (COMPOUND 5527)

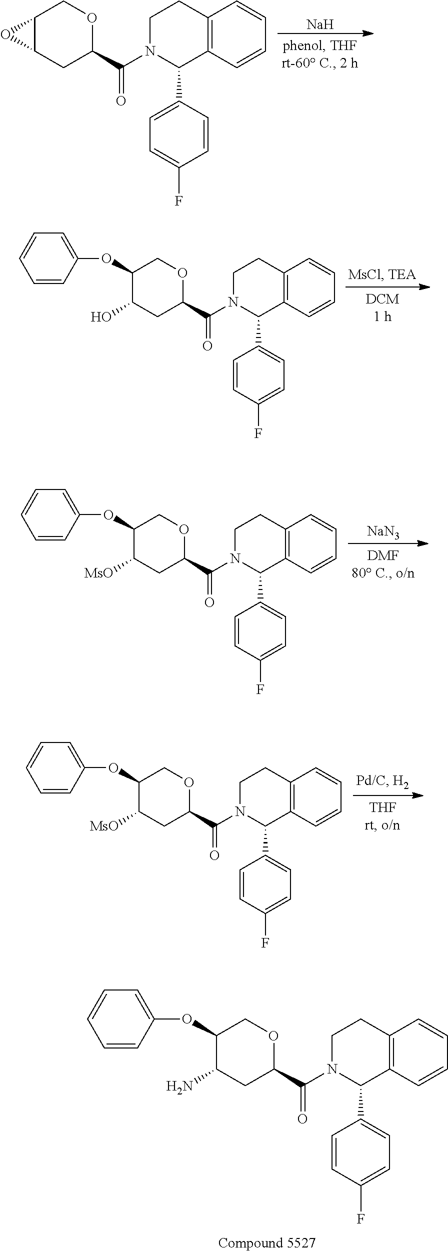

### Synthesis of ((S)-1-(4-fluorophenyl)-3,4-dihydroisoquinolin-2(1H)-yl)((2R,4S,5S)-4-hydroxy-5-phenoxytetrahydro-2H-pyran-2-yl)methanone

Sodium hydride (60 wt % dispersion in mineral oil, 28.3 mg, 0.707 mmol) was added to phenol (666 mg, 7.07 mmol). After 5 minutes, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 100 mg, 0.283 mmol) was added and the reaction mixture was warmed to 60° C. After 1 hour, the mixture was diluted with tetrahydrofuran (dry, 1 mL). After another hour the mixture was cooled to room temperature, diluted with dichloromethane (3 mL), and washed with saturated aqueous NH₄Cl (3 mL). The organic layer was passed through a phase separator and evaporated under reduced pressure. The residue was purified by flash column chromatography (silica, 0 to 100% ethyl acetate in heptane) to give ((S)-1-(4-fluorophenyl)-3,4-dihydroisoquinolin-2(1H)-yl)((2R,4S,5S)-4-hydroxy-5-phenoxytetrahydro-2H-pyran-2-yl)methanone.

LCMS: 96%, RT=2.11 min., (M+H)⁺=448 (method A).

### Synthesis of (2R,4S,5S)-2-((S)-1-(4-fluorophenyl)-1,2,3,4-tetrahydroisoquinoline-2-carbonyl)-5-phenoxytetrahydro-2H-pyran-4-yl methanesulfonate

Triethylamine (0.105 mL, 0.754 mmol) followed by methanesulfonyl chloride (0.029 mL, 0.377 mmol) were added to a solution of ((S)-1-(4-fluorophenyl)-3,4-dihydroisoquinolin-2(1H)-yl)((2R,4S,5S)-4-hydroxy-5-phenoxytetrahydro-2H-pyran-2-yl)methanone (100 mg, 0.223 mmol) in dichloromethane (4 mL). After 1 hour, the reaction mixture was washed with aqueous citric acid (0.5 M, 4 mL) and saturated aqueous NaHCO₃ (4 mL). The organic layer was passed through a phase separator and evaporated under reduced pressure to give (2R,4S,5S)-2-((S)-1-(4-fluorophenyl)-1,2,3,4-tetrahydroisoquinoline-2-carbonyl)-5-phenoxytetrahydro-2H-pyran-4-yl methanesulfonate.

LCMS: 96%, RT=2.20 min., (M+H)⁺=526 (method A).

### Synthesis of ((2R,4S,5R)-4-azido-5-phenoxytetrahydro-2H-pyran-2-yl)((S)-1-(4-fluorophenyl)-3,4-dihydroisoquinolin-2(1H)-yl)methanone

Sodium azide (25.2 mg, 0.388 mmol) was added to a solution of (2R,4S,5S)-2-((S)-1-(4-fluorophenyl)-1,2,3,4-tetrahydroisoquinoline-2-carbonyl)-5-phenoxytetrahydro-2H-pyran-4-yl methanesulfonate (102 mg, 0.194 mmol) in N,N-dimethylformamide (4 mL) after which the reaction mixture was warmed to 80° C. After stirring overnight, another portion of sodium azide (25.2 mg, 0.388 mmol) was added and stirring was continued for another hour. Then, the mixture was cooled to room temperature and partitioned between water and ethyl acetate. The organic layer was dried over Na₂SO₄ and evaporated under reduced pressure. The residue was purified by flash column chromatography (silica, 0 to 100% ethyl acetate in heptane) to give ((2R,4S,5R)-4-azido-5-phenoxytetrahydro-2H-pyran-2-yl)((S)-1-(4-fluorophenyl)-3,4-dihydroisoquinolin-2(1H)-yl)methanone.

LCMS: 99%, RT=2.31 min., (M+H)⁺=473 (method A).

### Synthesis of ((2R,4S,5R)-4-amino-5-phenoxytetrahydro-2H-pyran-2-yl)((S)-1-(4-fluorophenyl)-3,4-dihydroisoquinolin-2(1H)-yl)methanone (COMPOUND 5527)

A solution of ((2R,4S,5R)-4-azido-5-phenoxytetrahydro-2H-pyran-2-yl)((S)-1-(4-fluorophenyl)-3,4-dihydroisoquinolin-2(1H)-yl)methanone (60 mg, 0.127 mmol) in tetrahydrofuran (4 mL) was hydrogenated in the presence of palladium (10 wt % on carbon, containing 50% water, 27.0 mg, 0.013 mmol) at atmospheric hydrogen pressure. After stirring overnight, the mixture was filtered over a 0.22 μm nylon filter. The filtrate was concentrated under reduced pressure. The residue was dissolved in methanol, loaded onto an SCX-2 column (1 g), and eluted with methanol (5 column volumes). Next, the column was eluted with ammonia in methanol (2 M). The basic fraction was concentrated to dryness under reduced pressure to give ((2R,4S,5R)-4-amino-5-phenoxytetrahydro-2H-pyran-2-yl)((S)-1-(4-fluorophenyl)-3,4-dihydroisoquinolin-2(1H)-yl)methanone (COMPOUND 5527) after lyophilization from a mixture of acetonitrile and water.

LCMS: 99%, RT=1.26 min., (M+H)⁺=447 (method P).

### Example 128

### ((2R,4S,5R)-4-amino-5-cyclobutoxytetrahydro-2H-pyran-2-yl)((S)-1-(4-fluorophenyl)-3,4-dihydroisoquinolin-2(1H)-yl)methanone (COMPOUND 5525)

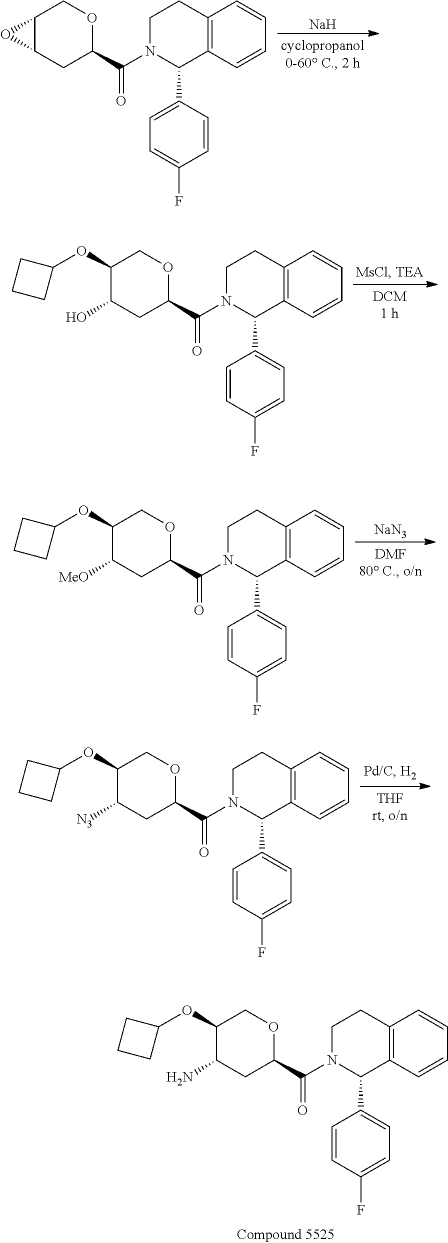

### Synthesis of ((2R,4S,5S)-5-cyclobutoxy-4-hydroxytetrahydro-2H-pyran-2-yl)((S)-1-(4-fluorophenyl)-3,4-dihydroisoquinolin-2(1H)-yl)methanone

Sodium hydride (60 wt % dispersion in mineral oil, 28.3 mg, 0.707 mmol) was added to cyclobutanol (554 μL, 7.07 mmol). After 5 minutes, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 100 mg, 0.283 mmol) was added and the reaction mixture was warmed to 60° C. After 2 hours, the mixture was cooled to room temperature, diluted with dichloromethane (3 mL), and washed with saturated aqueous NH₄Cl (3 mL). The organics layer was passed through a phase separator and evaporated under reduced pressure. The residue was purified by flash column chromatography (silica, 0 to 100% ethyl acetate in heptane) to give ((2R,4S,5S)-5-cyclobutoxy-4-hydroxytetrahydro-2H-pyran-2-yl)((S)-1-(4-fluorophenyl)-3,4-dihydroisoquinolin-2(1H)-yl)methanone.

LCMS: 97%, RT=2.04 min., (M+H)⁺=426 (method A).

### Synthesis of (2R,4S,5S)-5-cyclobutoxy-2-((S)-1-(4-fluorophenyl)-1,2,3,4-tetrahydroisoquinoline-2-carbonyl)tetrahydro-2H-pyran-4-yl methanesulfonate

Triethylamine (0.105 mL, 0.754 mmol) followed by methanesulfonyl chloride (0.029 mL, 0.377 mmol) were added to a solution of ((2R,4S,5S)-5-cyclobutoxy-4-hydroxytetrahydro-2H-pyran-2-yl)((S)-1-(4-fluorophenyl)-3,4-dihydroisoquinolin-2(1H)-yl)methanone (107 mg, 0.251 mmol) in dichloromethane (4 mL). After 1 hour, the reaction mixture was washed with aqueous citric acid (0.5 M, 4 mL) and saturated aqueous NaHCO₃ (4 mL). The organic layer was passed through a phase separator and evaporated under reduced pressure to give (2R,4S,5S)-5-cyclobutoxy-2-((S)-1-(4-fluorophenyl)-1,2,3,4-tetrahydroisoquinoline-2-carbonyl)tetrahydro-2H-pyran-4-yl methanesulfonate.

LCMS: 95%, RT=2.18 min., (M+H)⁺=504 (method A).

### Synthesis of ((2R,4S,5R)-4-azido-5-cyclobutoxytetrahydro-2H-pyran-2-yl)((S)-1-(4-fluorophenyl)-3,4-dihydroisoquinolin-2(1H)-yl)methanone

Sodium azide (28.9 mg, 0.445 mmol) was added to a solution of (2R,4S,5S)-5-cyclobutoxy-2-((S)-1-(4-fluorophenyl)-1,2,3,4-tetrahydroisoquinoline-2-carbonyl)tetrahydro-2H-pyran-4-yl methanesulfonate (112 mg, 0.222 mmol) in N,N-dimethylformamide (4 mL) after which the reaction mixture was warmed to 80° C. After stirring overnight, the mixture was cooled to room temperature and partitioned between water and ethyl acetate. The organic layer was dried over Na₂SO₄ and evaporated under reduced pressure. The residue was purified by flash column chromatography (silica, 0 to 100% ethyl acetate in heptane) to give ((2R,4S,5R)-4-azido-5-cyclobutoxytetrahydro-2H-pyran-2-yl)((S)-1-(4-fluorophenyl)-3,4-dihydroisoquinolin-2(1H)-yl)methanone.

LCMS: 100%, RT=2.29 min., (M+H)⁺=451 (method A).

### Synthesis of ((2R,4S,5R)-4-amino-5-cyclobutoxytetrahydro-2H-pyran-2-yl)((S)-1-(4-fluorophenyl)-3,4-dihydroisoquinolin-2(1H)-yl)methanone (COMPOUND 5525)

A solution of ((2R,4S,5R)-4-azido-5-cyclobutoxytetrahydro-2H-pyran-2-yl)((S)-1-(4-fluorophenyl)-3,4-dihydroisoquinolin-2(1H)-yl)methanone (65 mg, 0.144 mmol) in tetrahydrofuran (4 mL) was hydrogenated in the presence of palladium (10 wt % on carbon, containing 50% water, 6.14 mg, 2.89 μmol) at atmospheric hydrogen pressure. After stirring overnight, the mixture was filtered over a 0.22 μm nylon filter. The filtrate was concentrated under reduced pressure. The residue was dissolved in methanol, loaded onto an SCX-2 column (1 g), and eluted with methanol (5 column volumes). Next, the column was eluted with ammonia in methanol (2 M). The basic fraction was concentrated to dryness under reduced pressure to give ((2R,4S,5R)-4-amino-5-cyclobutoxytetrahydro-2H-pyran-2-yl)((S)-1-(4-fluorophenyl)-3,4-dihydroisoquinolin-2(1H)-yl)methanone (COMPOUND 5525) after lyophilization from a mixture of acetonitrile and water.

LCMS: 98%, RT=1.20 min., (M+H)⁺=425 (method P).

### Example 129

### ((2R,4S,5R)-4-amino-5-ethyltetrahydro-2H-pyran-2-yl)((S)-1-(4-fluorophenyl)-3,4-dihydroisoquinolin-2(1H)-yl)methanone (COMPOUND 5526)

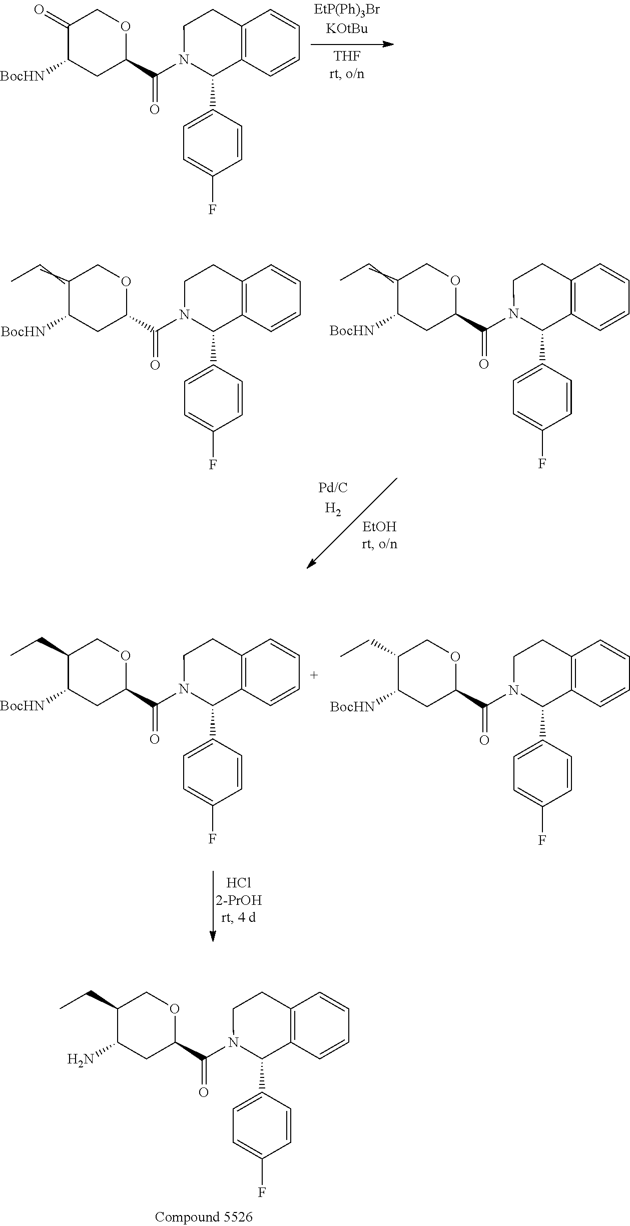

### Synthesis of tert-butyl ((2R,4S)-5-ethylidene-2-((S)-1-(4-fluorophenyl)-1,2,3,4-tetrahydroisoquinoline-2-carbonyl)tetrahydro-2H-pyran-4-yl)carbamate and tert-butyl ((2S,4S)-5-ethylidene-2-((S)-1-(4-fluorophenyl)-1,2,3,4-tetrahydroisoquinoline-2-carbonyl)tetrahydro-2H-pyran-4-yl)carbamate

At 0° C., Potassium tert-butoxide (1.0 M in tetrahydrofuran, 2.433 mL, 2.433 mmol) was added to a suspension of (ethyl)triphenylphosphonium bromide (1054 mg, 2.84 mmol) in tetrahydrofuran (dry, 10 mL). After 30 minutes, a solution of tert-butyl ((2R,4S)-2-((S)-1-(4-fluorophenyl)-1,2,3,4-tetrahydroisoquinoline-2-carbonyl)-5-oxotetrahydro-2H-pyran-4-yl)carbamate (see Compound 5518, 950 mg, 2.028 mmol) in tetrahydrofuran (dry, 5 mL) was added dropwise and stirring was continued overnight. Then, the reaction mixture was quenched with saturated aqueous ammonium chloride (10 mL) and diluted with ethyl acetate (50 mL). The mixture was filtered and the layers were separated. The organic phase was dried over Na₂SO₄ and evaporated under reduced pressure. The residue was purified by flash column chromatography (silica, 0 to 50% ethyl acetate in heptane) and preparative SFC (method BT) to give ((2S,4S)-5-ethylidene-2-((S)-1-(4-fluorophenyl)-1,2,3,4-tetrahydroisoquinoline-2-carbonyl)tetrahydro-2H-pyran-4-yl)carbamate as the first eluting isomer on silica and SFC and ((2R,4S)-5-ethylidene-2-((S)-1-(4-fluorophenyl)-1,2,3,4-tetrahydroisoquinoline-2-carbonyl)tetrahydro-2H-pyran-4-yl)carbamate as the second eluting isomer on silica and SFC.

First eluting isomer. SFC: 100%, RT=2.66 min., (M+H)⁺=481 (method F). LCMS: 94%, RT=2.28 min., (M+Na)⁺=503 (method A).

Second eluting isomer. SFC: 100%, RT=3.38 min., (M+H)⁺=481 (method F). LCMS: 100%, RT=2.19 min., (M+H)⁺=481 (method A).

### Synthesis of tert-butyl ((2R,4S,5R)-5-ethyl-2-((S)-1-(4-fluorophenyl)-1,2,3,4-tetrahydroisoquinoline-2-carbonyl)tetrahydro-2H-pyran-4-yl)carbamate

A solution of tert-butyl ((2R,4S)-5-ethylidene-2-((S)-1-(4-fluorophenyl)-1,2,3,4-tetrahydroisoquinoline-2-carbonyl)tetrahydro-2H-pyran-4-yl)carbamate (91 mg, 0.189 mmol) in ethanol (3 mL) was hydrogenated in the presence of palladium (10 wt % on carbon, 20.15 mg, 0.019 mmol) at atmospheric hydrogen pressure. After stirring overnight, the mixture was filtered over a 0.22 μm nylon filter and the filtrate was concentrated under reduced pressure. The residue was purified by preparative SFC (method AE) to give tert-butyl ((2R,4S,5R)-5-ethyl-2-((S)-1-(4-fluorophenyl)-1,2,3,4-tetrahydroisoquinoline-2-carbonyl)tetrahydro-2H-pyran-4-yl)carbamate as the first eluting isomer and tert-butyl ((2R,4S,5S)-5-ethyl-2-((S)-1-(4-fluorophenyl)-1,2,3,4-tetrahydroisoquinoline-2-carbonyl)tetrahydro-2H-pyran-4-yl)carbamate as the second eluting isomer.

First eluting isomer: SFC: 100%, RT=2.57 min., (M+H)⁺=483 (method AD). LCMS: 100%, RT=2.25 min., (M+H)⁺=483 (method A).

Second eluting isomer: SFC: 90%, RT=2.81 min., (M+H)⁺=483 (method AD). LCMS: 80%, RT=2.21 min., (M+H)⁺=483 (method A).

### Synthesis of ((2R,4S,5R)-4-amino-5-ethyltetrahydro-2H-pyran-2-yl)((S)-1-(4-fluorophenyl)-3,4-dihydroisoquinolin-2(1H)-yl)methanone (COMPOUND 5526)

HCl (6 M in 2-propanol, 0.15 mL, 0.90 mmol) was added to a solution of tert-butyl ((2R,4S,5R)-5-ethyl-2-((S)-1-(4-fluorophenyl)-1,2,3,4-tetrahydroisoquinoline-2-carbonyl)tetrahydro-2H-pyran-4-yl)carbamate (33 mg, 0.068 mmol) in 2-propanol (1.5 mL). After stirring for 4 days, the reaction mixture was diluted with dichloromethane (10 mL) and saturated aqueous NaHCO₃ (10 mL). The layers were separated using a phase-separator and the organic filtrate was evaporated under reduced pressure. The residue was dissolved in methanol (2 mL), brought onto an SCX-2 column (2 g), and eluted with methanol until neutral. Next, the column was eluted with ammonia in methanol (1.5 M). The basic fraction was concentrated to dryness under reduced pressure and lyophilized from a mixture of acetonitrile and water (1:2, 3 mL) to give ((2R,4S,5R)-4-amino-5-ethyltetrahydro-2H-pyran-2-yl)((S)-1-(4-fluorophenyl)-3,4-dihydroisoquinolin-2(1H)-yl)methanone (COMPOUND 5526).

LCMS: 99%, RT=1.14 min., (M+H)⁺=383 (method P).

### Example 130

### ((S)-1-(4-fluorophenyl)-3,4-dihydroisoquinolin-2(1H)-yl)((3aS,6R,7aS)-3-methyl-3,3a,4,6,7,7a-hexahydropyrano[3,4-d]imidazol-6-yl)methanone hydrochloride (COMPOUND 5528)

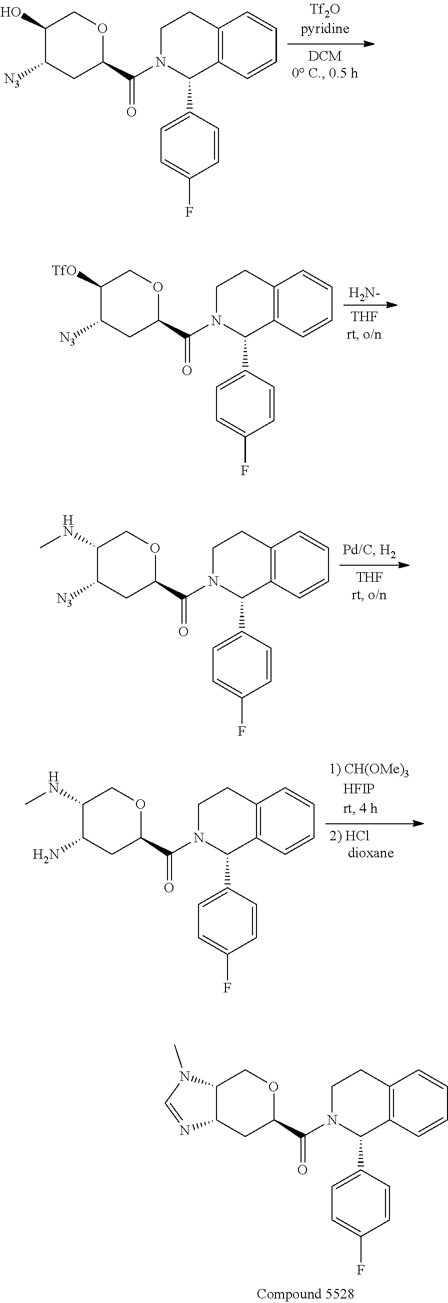

### Synthesis of (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate

Under an argon atmosphere at 0° C., pyridine (0.214 mL, 2.65 mmol) followed by trifluoromethanesulfonic anhydride (0.220 mL, 1.324 mmol) were added to a solution of ((2R,4S,5R)-4-azido-5-hydroxytetrahydro-2H-pyran-2-yl)((S)-1-(4-fluorophenyl)-3,4-dihydroisoquinolin-2(1H)-yl)methanone (see Compound 5077, 0.35 g, 0.883 mmol) in dichloromethane (8 mL). After 0.5 hour, the reaction mixture was diluted with dichloromethane (10 mL) and washed with aqueous citric acid (0.5 M, 10 mL) and saturated aqueous NaHCO₃ (10 mL). The separated organic layer was dried over Na₂SO₄ and evaporated under reduced pressure to give (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate.

LCMS: 100%, RT=2.28 min., (M+H)⁺=529 (Method A).

### Synthesis of ((2R,4S,5S)-4-azido-5-(methylamino)tetrahydro-2H-pyran-2-yl)((S)-1-(4-fluorophenyl)-3,4-dihydroisoquinolin-2(1H)-yl)methanone

(3R,4S,6R)-4-Azido-6-((S)-1-(4-fluorophenyl)-1,2,3,4-tetrahydroisoquinoline-2-carbonyl)tetrahydro-2H-pyran-3-yl trifluoromethanesulfonate (0.425 g, 0.804 mmol) was taken up in a solution of methylamine in tetrahydrofuran (2 M, 5.54 mL, 11.09 mmol). After stirring overnight, the reaction mixture was concentrated under reduced pressure. The residue was dissolved in methanol (1 mL) and brought onto an SCX-2 column (5 g) and eluted with methanol (5 column volumes). Next, the column was eluted with ammonia in methanol (7 M). The basic fraction was concentrated to dryness under reduced pressure to give ((2R,4S,5S)-4-azido-5-(methylamino)tetrahydro-2H-pyran-2-yl)((S)-1-(4-fluorophenyl)-3,4-dihydroisoquinolin-2(1H)-yl)methanone.

LCMS: 92%, RT=1.64 min., (M+H)⁺=410 (method A).

### Synthesis of ((2R,4S,5S)-4-amino-5-(methylamino)tetrahydro-2H-pyran-2-yl)((S)-1-(4-fluorophenyl)-3,4-dihydroisoquinolin-2(1H)-yl)methanone

A solution of ((2R,4S,5S)-4-azido-5-(methylamino)tetrahydro-2H-pyran-2-yl)((S)-1-(4-fluorophenyl)-3,4-dihydroisoquinolin-2(1H)-yl)methanone (0.144 g, 0.352 mmol) in tetrahydrofuran (3.5 mL) was hydrogenated in the presence of palladium (10 wt % on carbon, containing 50% water, 7.5 mg, 0.035 mmol) at atmospheric hydrogen pressure. After stirring overnight, the mixture was filtered over a 0.22 μm nylon filter. The filtrate was concentrated under reduced pressure. The residue was dissolved in methanol (1 mL) and brought onto an SCX-2 column (2 g) and eluted with methanol (5 column volumes). Next, the column was eluted with ammonia in methanol (7 M). The basic fraction was concentrated to dryness under reduced pressure to give ((2R,4S,5S)-4-amino-5-(methylamino)tetrahydro-2H-pyran-2-yl)((S)-1-(4-fluorophenyl)-3,4-dihydroisoquinolin-2(1H)-yl)methanone.

LCMS: 94%, RT=1.44 min., (M+H)⁺=384 (method A).

### Synthesis of ((S)-1-(4-fluorophenyl)-3,4-dihydroisoquinolin-2(1H)-yl)((3aS,6R,7aS)-3-methyl-3,3a,4,6,7,7a-hexahydropyrano[3,4-d]imidazol-6-yl)methanone hydrochloride (COMPOUND 5528)

Trimethyl orthoformate (0.061 mL, 0.554 mmol) was added to a solution of ((2R,4S,5S)-4-amino-5-(methylamino)tetrahydro-2H-pyran-2-yl)((S)-1-(4-fluorophenyl)-3,4-dihydroisoquinolin-2(1H)-yl)methanone (0.192 g, 0.501 mmol) in hexafluoroisopropanol (4.06 mL, 38.6 mmol). After 4 hours, the reaction mixture was concentrated under reduced pressure to give the product as the free base (˜250 mg; degrades in time). HCl (4 M in 1,4-dioxane, 0.063 mL, 0.250 mmol) was added to a solution of the free base (50 mg) till the pH was acidic on pH-paper. The mixture was concentrated under reduced pressure and the residue was lyophilized from a mixture of acetonitrile and water (1:1, 4 mL) to give ((S)-1-(4-fluorophenyl)-3,4-dihydroisoquinolin-2(1H)-yl)((3aS,6R,7aS)-3-methyl-3,3a,4,6,7,7a-hexahydropyrano[3,4-d]imidazol-6-yl)methanone hydrochloride.

LCMS: 92%, RT=1.04 min., (M+H)⁺=394 (method P).

### Example 131

### ((S)-1-(4-fluorophenyl)-3,4-dihydroisoquinolin-2(1H)-yl)((R)-3-methyl-3,4,6,7-tetrahydropyrano[3,4-d]imidazol-6-yl)methanone (COMPOUND 5529)

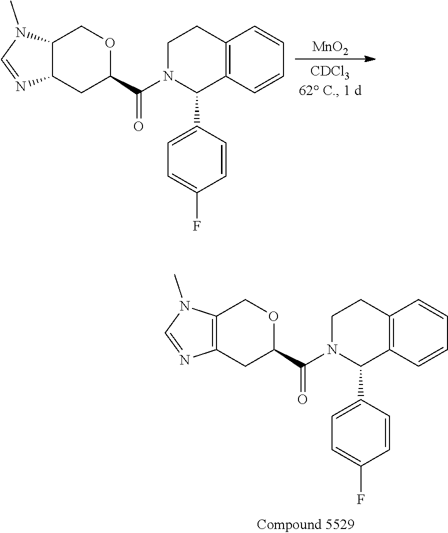

### Synthesis of ((S)-1-(4-fluorophenyl)-3,4-dihydroisoquinolin-2(1H)-yl)((R)-3-methyl-3,4,6,7-tetrahydropyrano[3,4-d]imidazol-6-yl)methanone (COMPOUND 5529)

In a screw-cap reaction vial under argon atmosphere, manganese(IV) oxide (110 mg, 1.271 mmol) was added to a solution of ((S)-1-(4-fluorophenyl)-3,4-dihydroisoquinolin-2(1H)-yl)((3aS,6R,7aS)-3-methyl-3,3a,4,6,7,7a-hexahydropyrano[3,4-d]imidazol-6-yl)methanone (see Compound 5528, 50 mg, 0.127 mmol) in chloroform-d (3 mL). The reaction vial was transferred into pre-heated reaction block (62° C.) and stirred overnight. An additional amount of manganese(IV) oxide (110 mg, 1.271 mmol) was added and stirring was continued for 5 hours. After cooling to room temperature, the reaction mixture was filtered through a 0.45 Om filter and the filtrate was concentrated under reduced pressure. The residue was purified by acidic preparative MPLC (Linear Gradient: t=0 min 5% A, t=1 min 5% A; t=16 min 50% A; t=17 min 100%; t=22 min 100% A; detection: 220/254 nm) to give ((S)-1-(4-fluorophenyl)-3,4-dihydroisoquinolin-2(1H)-yl)((R)-3-methyl-3,4,6,7-tetrahydropyrano[3,4-d]imidazol-6-yl)methanone after lyophilization from a mixture of acetonitrile and water (1:1, 4 mL).

LCMS: 100%, RT=1.07 min., (M+H)⁺=392 (method P).

### Example 132

### (4aR,7R,8aS)-7-((S)-1-(4-fluorophenyl)-1,2,3,4-tetrahydroisoquinoline-2-carbonyl)-4-methylhexahydro-2H-pyrano[3,4-b]pyrazin-3(4H)-one (COMPOUND 5533)

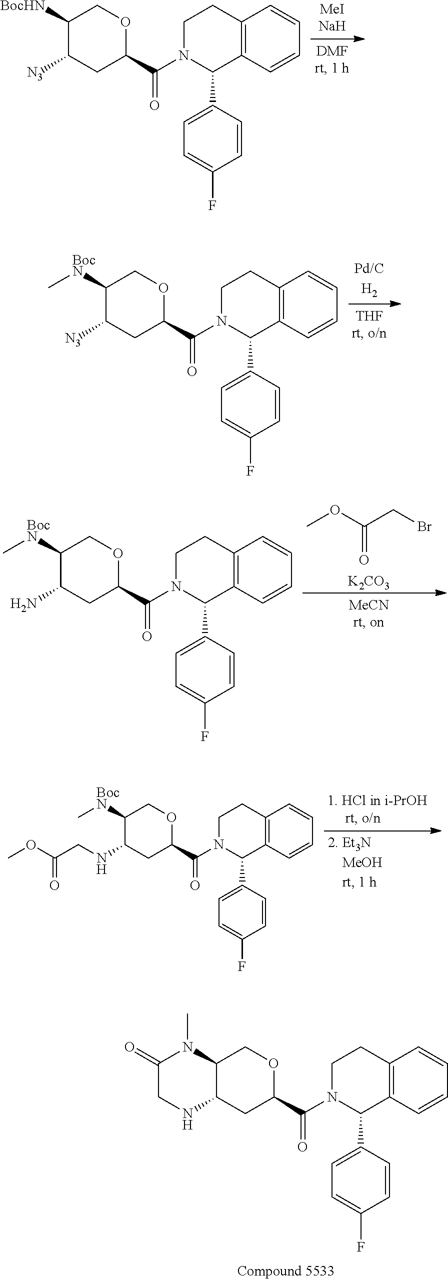

### Synthesis of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate

Under nitrogen atmosphere, sodium hydride (60% dispersion in mineral oil, 38.7 mg, 0.969 mmol) was added to a solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate (see COMPOUND 5532, 400 mg, 0.807 mmol) in N,N-dimethylformamide (dry, 4 mL) After 15 minutes, methyliodide (0.176 mL, 2.83 mmol) was added dropwise and stirring was continued for 1 hour. Then, the reaction mixture was diluted with ethyl acetate (30 mL) and brine (15 mL). The aqueous layer was separated and extracted with ethyl acetate (20 mL). The combined organics were washed with brine (2×10 mL), dried on Na₂SO₄, and concentrated under reduced pressure. The residue was purified by flash column chromatography (silica, 0 to 40% ethyl acetate in heptane) to give tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate.

LCMS: 99%, RT=2.30 min., (M+H)⁺=510 (method A).

### Synthesis of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(methyl)carbamate

A solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(methyl)carbamate (156 mg, 0.306 mmol) in tetrahydrofuran (4 mL) was hydrogenated in the presence of palladium (10 wt % on carbon, containing 50% water, 80 mg, 0.038 mmol) at atmospheric hydrogen pressure. After stirring overnight, the mixture was diluted with ethyl acetate and filtered over a 0.22 μm nylon filter. The filtrate was concentrated under reduced pressure and co-evaporated with dichloromethane to give tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(methyl)carbamate.

LCMS: 99%, RT=1.77 min., (M+H)⁺=484 (method A).

### Synthesis of methyl ((2R,4S,5R)-5-((tert-butoxycarbonyl)(methyl)amino)-2-((S)-1-(4-fluorophenyl)-1,2,3,4-tetrahydroisoquinoline-2-carbonyl)tetrahydro-2H-pyran-4-yl)glycinate

Methyl bromoacetate (0.058 mL, 0.612 mmol) was added to a suspension of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(methyl)carbamate (148 mg, 0.306 mmol) and potassium carbonate (127 mg, 0.918 mmol) in acetonitrile (2.5 mL). After stirring overnight, the mixture was diluted with dichloromethane and water and the layers were separated over a phase separation filter. The organic filtrate was concentrated under reduced pressure and purified by flash column chromatography (silica, 10 to 100% ethyl acetate in heptane) to give methyl ((2R,4S,5R)-5-((tert-butoxycarbonyl)(methyl)amino)-2-((S)-1-(4-fluorophenyl)-1,2,3,4-tetrahydroisoquinoline-2-carbonyl)tetrahydro-2H-pyran-4-yl)glycinate after co-evaporation from dichloromethane.

LCMS: 97%, RT=1.82 min., (M+H)⁺=556 (method A).

### Synthesis of (4aR,7R,8aS)-7-((S)-1-(4-fluorophenyl)-1,2,3,4-tetrahydroisoquinoline-2-carbonyl)-4-methylhexahydro-2H-pyrano[3,4-b]pyrazin-3(4H)-one (COMPOUND 5533)

HCl (5-6 M in 2-propanol, 0.5 mL, 3.00 mmol) was added to a solution of methyl ((2R,4S,5R)-5-((tert-butoxycarbonyl)(methyl)amino)-2-((S)-1-(4-fluorophenyl)-1,2,3,4-tetrahydroisoquinoline-2-carbonyl)tetrahydro-2H-pyran-4-yl)glycinate (110 mg, 0.198 mmol) in 2-propanol (2 mL). After stirring overnight, the mixture was diluted with dichloromethane (10 mL) and saturated aqueous NaHCO₃ (10 mL) and stirred for 1 hour. The layers were separated over a phase separation filter and the organic filtrate was concentrated under reduced pressure. The residue was taken up in a mixture of methanol (32 mL) and dichloromethane (1 mL) and triethylamine (0.041 ml, 0.297 mmol) was added. After stirring for 1 hour, the mixture was concentrated to dryness under reduced pressure. The residue was purified by flash column chromatography (silica, 1 to 10% (7 M NH₃) methanol) in dichloromethane) to give (4aR,7R,8aS)-7-((S)-1-(4-fluorophenyl)-1,2,3,4-tetrahydroisoquinoline-2-carbonyl)-4-methylhexahydro-2H-pyrano[3,4-b]pyrazin-3(4H)-one (COMPOUND 5533) after lyophilization from a mixture of acetonitrile and water (1:1, 4 mL).

LCMS: 97%, RT=1.07 min., (M+H)⁺=424 (method P).

### Example 133

### (4aR,7R,8aS)-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5532)

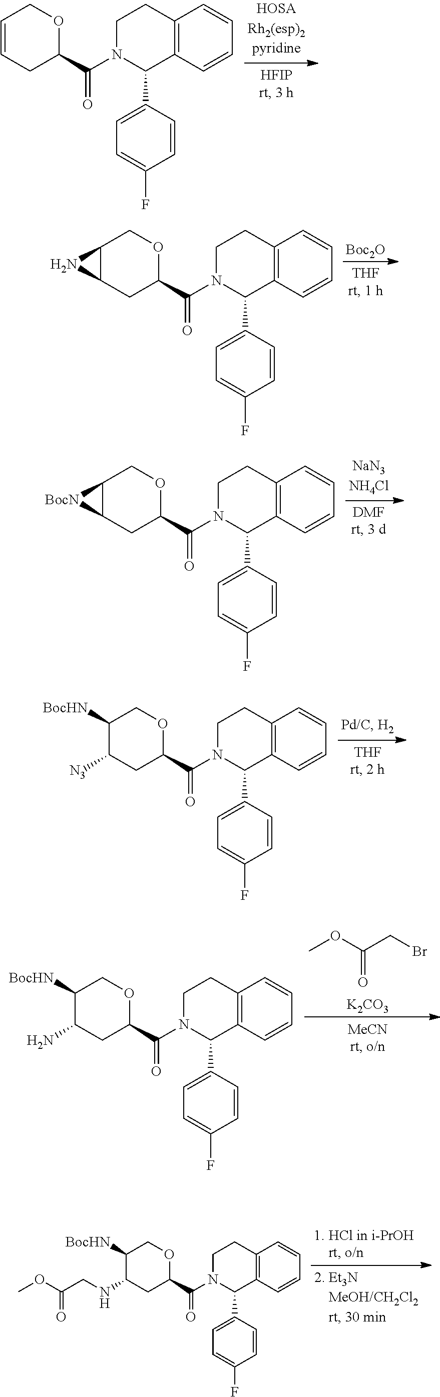

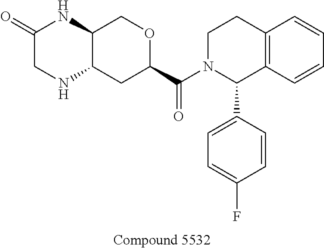 [SMILES: O=C1CN[C@H]2C[C@H](C(=O)N3CCc4ccccc4[C@@H]3c3ccc(F)cc3)OC[C@@H]2N1]

### Synthesis of ((1R,4R,6R)-3-oxa-7-azabicyclo[4.1.0]heptan-4-yl)((S)-1-(4-fluorophenyl)-3,4-dihydroisoquinolin-2(1H)-yl)methanone

Pyridine (1.031 mL, 12.74 mmol), hydroxylamine-O-sulfonic acid (0.865 g, 7.65 mmol) and bis[rhodium(α,α,α′,α′-tetramethyl-1,3-benzenedipropanoic acid)] (0.097 g, 0.127 mmol) were added to a solution of ((R)-3,6-dihydro-2H-pyran-2-yl)((S)-1-(4-fluorophenyl)-3,4-dihydroisoquinolin-2(1H)-yl)methanone (see Compound 5053, 2.15 g, 6.37 mmol) in hexafluoro-2-propanol (32.25 mL). After stirring for 3 hours, the reaction mixture was diluted with saturated aqueous NaHCO₃ (20 mL) and extracted with dichloromethane (3×30 mL). The combined organics were dried over Na₂SO₄ and evaporated under reduced pressure. The residue was purified by flash column chromatography (silica, 0 to 5% (7 M ammonia in methanol) in dichloromethane) to give ((1R,4R,6R)-3-oxa-7-azabicyclo[4.1.0]heptan-4-yl)((S)-1-(4-fluorophenyl)-3,4-dihydroisoquinolin-2(1H)-yl)methanone.

LCMS: 99%, RT=1.59 min., (M+H)⁺=353 (method A).

### Synthesis of tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate

Di-tert-butyl dicarbonate (1.61 g, 7.38 mmol) was added to a solution of ((1R,4R,6R)-3-oxa-7-azabicyclo[4.1.0]heptan-4-yl)((S)-1-(4-fluorophenyl)-3,4-dihydroisoquinolin-2(1H)-yl)methanone (1.300 g, 3.69 mmol) in tetrahydrofuran (35 mL). After 1 hour, the mixture was evaporated under reduced pressure. The residue was purified by flash column chromatography (silica, 0 to 50% ethyl acetate in heptane) to give tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate.

LCMS: 99%, RT=2.15 min., (M+H)⁺=453 (method B).

### Synthesis of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate

Under argon atmosphere, ammonium chloride (0.296 g, 5.52 mmol) followed by sodium azide (0.539 g, 8.29 mmol) were added to a solution of tert-butyl (1R,4R,6R)-4-((S)-1-(4-fluorophenyl)-1,2,3,4-tetrahydroisoquinoline-2-carbonyl)-3-oxa-7-azabicyclo[4.1.0]heptane-7-carboxylate (1.25 g, 2.76 mmol) in N,N-dimethylformamide (dry, 20 mL). After stirring the reaction mixture over the weekend, it was diluted with brine (40 mL) and ethyl acetate (30 mL). The layers were separated and the aqueous phase was extracted with ethyl acetate (2×30 mL). The combined organics were washed with brine (25 mL), dried over Na₂SO₄, and evaporated under reduced pressure. The residue was purified by flash column chromatography (silica, 0 to 35% ethyl acetate in heptane) to give tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate.

LCMS: 100%, RT=2.23 min., (M+H)⁺=496 (method A).

### Synthesis of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)carbamate

A solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate (0.30 g, 0.605 mmol) in tetrahydrofuran (6 mL) was hydrogenated in the presence of palladium (10 wt % on carbon, containing 50% water, 0.129 g, 0.061 mmol) at atmospheric hydrogen pressure. After stirring for 2 hours, the mixture was filtered over a 0.22 μm nylon filter. The filtrate was concentrated under reduced pressure. The residue was dissolved in methanol, brought onto an SCX-2 column (5 g), and eluted with methanol (5 column volumes). Next, the column was eluted with ammonia in methanol (7 M). The basic fraction was concentrated to dryness under reduced pressure to give tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)carbamate.

LCMS: 97%, RT=1.82 min., (M+H)⁺=470 (method A).

### Synthesis of methyl ((2R,4S,5R)-5-((tert-butoxycarbonyl)amino)-2-((S)-1-(4-fluorophenyl)-1,2,3,4-tetrahydroisoquinoline-2-carbonyl)tetrahydro-2H-pyran-4-yl)glycinate

Methyl bromoacetate (0.024 mL, 0.256 mmol) was added to a suspension of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)carbamate (120 mg, 0.256 mmol) and potassium carbonate (70.6 mg, 0.511 mmol) in acetonitrile (0.5 mL). After 2 hours, additional potassium carbonate (35.3 mg, 0.256 mmol) and methyl bromoacetate (0.024 mL, 0.256 mmol) were added and stirring was continued overnight. Then, the mixture was diluted with dichloromethane and water and the layers were separated over a phase separation filter. The organic filtrate was concentrated under reduced pressure and purified by flash column chromatography (silica, 10 to 75% ethyl acetate in heptane) to give methyl ((2R,4S,5R)-5-((tert-butoxycarbonyl)amino)-2-((S)-1-(4-fluorophenyl)-1,2,3,4-tetrahydroisoquinoline-2-carbonyl)tetrahydro-2H-pyran-4-yl)glycinate after co-evaporation from dichloromethane.

LCMS: 98%, RT=2.13 min., (M+H)⁺=542 (method B).

### Synthesis of (4aR,7R,8aS)-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5532)

HCl (5-6 M in 2-propanol, 0.3 mL, 1.800 mmol) was added to a solution of methyl ((2R,4S,5R)-5-((tert-butoxycarbonyl)amino)-2-((S)-1-(4-fluorophenyl)-1,2,3,4-tetrahydroisoquinoline-2-carbonyl)tetrahydro-2H-pyran-4-yl)glycinate (86 mg, 0.159 mmol) in 2-propanol (1.5 mL). After stirring overnight, the mixture was diluted with dichloromethane (10 mL) and saturated aqueous NaHCO₃ (10 mL) and stirred for 10 minutes. The layers were separated over a phase separation filter and the organic filtrate was concentrated under reduced pressure. The residue was taken up in a mixture of methanol (2 mL) and dichloromethane (1 mL) and triethylamine (0.022 mL, 0.159 mmol) was added. After stirring the mixture for 1 hour, dichloromethane was removed under reduced pressure until a precipitate started to form. The mixture was left standing for about 30 minutes. The formed solids were filtered off and lyophilized from a mixture of acetonitrile and water to give (4aR,7R,8aS)-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5532).

LCMS: 99%, RT=1.02 min., (M+H)⁺=410 (method P).

### Example 134

### ((S)-1-(4-fluorophenyl)-3,4-dihydroisoquinolin-2(1H)-yl)((4aR,7R,8aS)-4-methyloctahydro-2H-pyrano[3,4-b]pyrazin-7-yl)methanone (COMPOUND 5462)

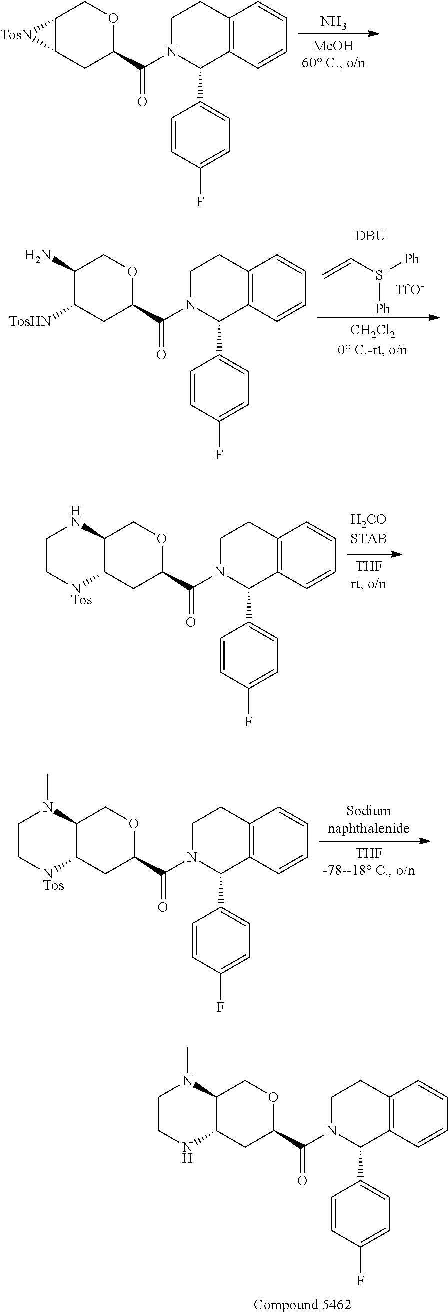

### Synthesis of N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide

A solution of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 550 mg, 1.086 mmol) in ammonia (7 M in methanol, 9 mL, 63 mmol) was heated to 60° C. After stirring overnight, the reaction mixture was concentrated under reduced pressure to give N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 86%, RT=1.72 min., (M+H)⁺=524 (method A).

### Synthesis of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone

At 0° C. under nitrogen atmosphere, 1,8-diazabicyclo[5.4.0]undec-7-ene (0.274 mL, 1.833 mmol) followed by ethenyldiphenylsulfanium trifluoromethanesulfonate (0.415 mL, 1.146 mmol) were added to a suspension of N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide (480 mg, 0.917 mmol) in dichloromethane (dry, 40 mL). After the addition was complete the reaction mixture was allowed to slowly warm to room temperature and stirred overnight. Then, the mixture was washed with aqueous HCl (1 M, 20 mL) and saturated aqueous NaHCO₃ (20 mL) and passed through a hydrophobic frit. The filtrate was evaporated under reduced pressure. The residue was purified by flash column chromatography (silica, 0 to 100% (ethyl acetate:methanol=9:1) in heptane) to give ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone.

LCMS: 94%, RT=1.78 min., (M+H)⁺=550 (method A).

### Synthesis of ((S)-1-(4-fluorophenyl)-3,4-dihydroisoquinolin-2(1H)-yl)((4aR,7R,8aS)-4-methyl-1-tosyloctahydro-2H-pyrano[3,4-b]pyrazin-7-yl)methanone

Formaldehyde (37 wt % solution in water, stabilized with 5-15% methanol, 0.031 mL, 0.409 mmol), sodium triacetoxyborohydride (87 mg, 0.409 mmol) and acetic acid (3.94 μL, 0.068 mmol) were added to a solution of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone (75 mg, 0.136 mmol) in tetrahydrofuran (1.5 mL). After stirring overnight, the mixture was combined with the reaction mixture of the same reaction (0.045 mmol), diluted with dichloromethane (15 mL), and washed with saturated aqueous NaHCO₃. The aqueous layer was extracted with dichloromethane (10 mL). The combined organic layers were passed through a hydrophobic frit and the filtrate was evaporated under reduced pressure to give ((S)-1-(4-fluorophenyl)-3,4-dihydroisoquinolin-2(1H)-yl)((4aR,7R,8aS)-4-methyl-1-tosyloctahydro-2H-pyrano[3,4-b]pyrazin-7-yl)methanone.

LCMS: 93%, RT=1.87 min., (M+H)⁺=564 (method A).

### Synthesis of ((S)-1-(4-fluorophenyl)-3,4-dihydroisoquinolin-2(1H)-yl)((4aR,7R,8aS)-4-methyloctahydro-2H-pyrano[3,4-b]pyrazin-7-yl)methanone (COMPOUND 5462)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.653 mL, 0.326 mmol) was added to a suspension of ((S)-1-(4-fluorophenyl)-3,4-dihydroisoquinolin-2(1H)-yl)((4aR,7R,8aS)-4-methyl-1-tosyloctahydro-2H-pyrano[3,4-b]pyrazin-7-yl)methanone (92 mg, 0.163 mmol) in tetrahydrofuran (dry, 1.25 mL). After 15 minutes, another portion of sodium naphthalenide (0.5M in tetrahydrofuran, 0.653 mL, 0.326 mmol) was added and after 15 minutes the reaction mixture was stored in the freezer overnight. Then, the mixture was diluted with dichloromethane (10 mL) and saturated aqueous NH₄Cl (5 mL). The layers were separated and the aqueous phase was extracted with dichloromethane (2×5 mL). The combined organic layers were passed through a hydrophobic frit and evaporated under reduced pressure. The residue was purified by flash column chromatography (silica, 0 to 10% (7M NH₃ in methanol) in dichloromethane and preparative SFC (method S). The product was dissolved in methanol (1 mL), brought onto an SCX-2 column (1 g), and eluted with methanol (˜5 column volumes). Next, the column was eluted with ammonia in methanol (1 M). The basic fraction was concentrated to dryness under reduced pressure to give ((S)-1-(4-fluorophenyl)-3,4-dihydroisoquinolin-2(1H)-yl)((4aR,7R,8aS)-4-methyloctahydro-2H-pyrano[3,4-b]pyrazin-7-yl)methanone (COMPOUND 5462) after lyophilization from a mixture of acetonitrile and water (1:1, 4 mL).

LCMS: 97%, RT=1.02 min., (M+H)⁺=410 (method P).

### Example 135

### ((S)-1-(4-fluorophenyl)-3,4-dihydroisoquinolin-2(1H)-yl)((4aR,7R,8aS)-octahydro-2H-pyrano[3,4-b]pyrazin-7-yl)methanone (COMPOUND 5461)

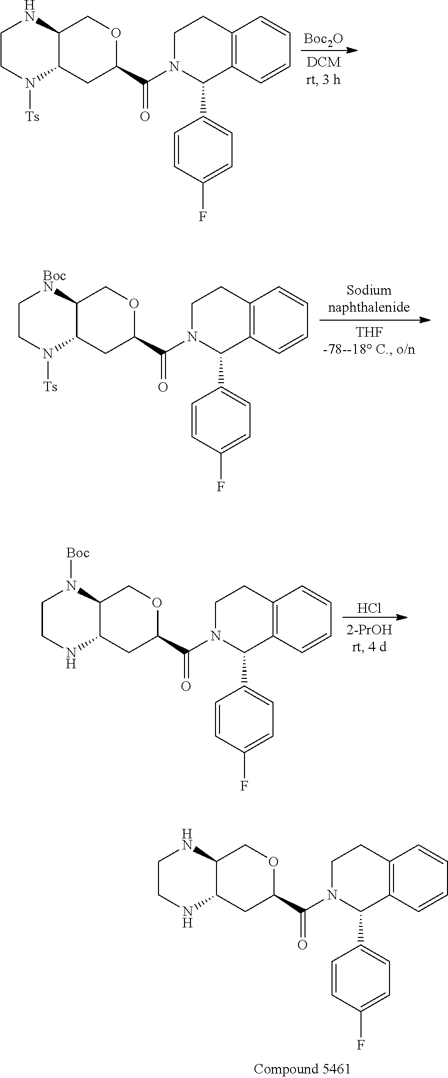 [SMILES: "Boc2O" ; Cc1ccc(S(=O)(=O)N2CCN[C@H]3CO[C@@H](C(=O)N4CCc5ccccc5[C@@H]4c4ccc(F)cc4)C[C@@H]32)cc1 ; Cc1ccc(S(=O)(=O)N2CCN(C(=O)OC(C)(C)C)[C@H]3CO[C@@H](C(=O)N4CCc5ccccc5[C@@H]4c4ccc(F)cc4)C[C@@H]32)cc1 ; CC(C)(C)OC(=O)N1CCN[C@H]2C[C@H](C(=O)N3CCc4ccccc4[C@@H]3c3ccc(F)cc3)OC[C@@H]21 ; Cl ; O=C([C@H]1C[C@@H]2NCCN[C@H]2CO1)N1CCc2ccccc2[C@@H]1c1ccc(F)cc1]

### Synthesis of tert-butyl (4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazine-4-carboxylate

Di-tert-butyl dicarbonate (50.2 mg, 0.230 mmol) was added to a solution of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone (see Compound 5462, 115 mg, 0.209 mmol) in dichloromethane (1.0 mL). After 3 hours, the reaction mixture was concentrated to dryness under reduced pressure to give tert-butyl (4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazine-4-carboxylate which was used as such.

### Synthesis of tert-butyl (4aR,7R,8aS)-7-((S)-1-(4-fluorophenyl)-1,2,3,4-tetrahydroisoquinoline-2-carbonyl)octahydro-4H-pyrano[3,4-b]pyrazine-4-carboxylate

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.739 mL, 0.369 mmol) was added to a solution of tert-butyl (4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazine-4-carboxylate (120 mg) in tetrahydrofuran (dry, 1.0 mL). After 15 minutes, the reaction mixture was stored in the freezer overnight. Then, the mixture was diluted with dichloromethane (10 mL) and saturated aqueous NH₄Cl (5 mL). The layers were separated and the aqueous phase was extracted with dichloromethane (2×5 mL). The combined organic layers were passed through a hydrophobic frit and evaporated under reduced pressure. The residue was purified by flash column chromatography (silica, 0 to 10% (7M NH₃ in methanol) in dichloromethane and preparative SFC (method S) and preparative SFC (method S). The product was dissolved in methanol (1 mL), brought onto an SCX-2 column (1 g), and eluted with methanol (˜5 column volumes). Next, the column was eluted with ammonia in methanol (1 M). The basic fraction was concentrated to dryness under reduced pressure to give tert-butyl (4aR,7R,8aS)-7-((S)-1-(4-fluorophenyl)-1,2,3,4-tetrahydroisoquinoline-2-carbonyl)octahydro-4H-pyrano[3,4-b]pyrazine-4-carboxylate.

LCMS: 96%, RT=1.97 min., (M+H)⁺=496 (method A).

### Synthesis of ((S)-1-(4-fluorophenyl)-3,4-dihydroisoquinolin-2(1H)-yl)((4aR,7R,8aS)-octahydro-2H-pyrano[3,4-b]pyrazin-7-yl)methanone (COMPOUND 5461)

HCl (6 M in 2-propanol, 0.5 mL, 3.00 mmol) was added to a solution of tert-butyl (4aR,7R,8aS)-7-((S)-1-(4-fluorophenyl)-1,2,3,4-tetrahydroisoquinoline-2-carbonyl)octahydro-4H-pyrano[3,4-b]pyrazine-4-carboxylate in 2-propanol (1.5 mL). After 2.5 hours, the reaction mixture was diluted with a mixture of chloroform-d (0.7 mL) and dichloromethane (1 mL) and stirring was continued overnight. Another portion of HCl (6 M in 2-propanol, 0.25 mL, 1.50 mmol) was added and stirring was continued for 3 more days. Then, the mixture was partitioned between dichloromethane (25 mL) and saturated aqueous NaHCO₃ (10 mL). The organic layer was passed through a hydrophobic frit and evaporated under reduced pressure. The residue was dissolved in a minimum amount of methanol, brought onto an SCX-2 column (1 g), and eluted with methanol (˜5 column volumes). Next, the column was eluted with ammonia in methanol (1 M). The basic fraction was concentrated to dryness under reduced pressure to give ((S)-1-(4-fluorophenyl)-3,4-dihydroisoquinolin-2(1H)-yl)((4aR,7R,8aS)-octahydro-2H-pyrano[3,4-b]pyrazin-7-yl)methanone (COMPOUND 5461) after lyophilization from a mixture of acetonitrile and water (1:1, 4 mL).

LCMS: 98%, RT=0.83 min., (M+H)⁺=396 (method P).

### Example 136

### ((S)-1-(4-fluorophenyl)-3,4-dihydroisoquinolin-2(1H)-yl)((4aR,7R,8aS)-octahydropyrano[3,4-b][1,4]thiazin-7-yl)methanone (COMPOUND 5534)

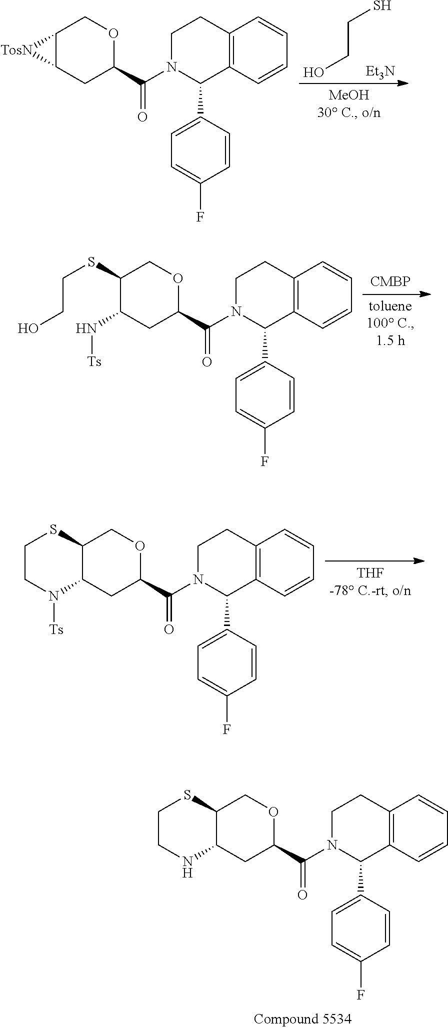

### Synthesis of N-((2R,4S,5R)-2-((S)-1-(4-fluorophenyl)-1,2,3,4-tetrahydroisoquinoline-2-carbonyl)-5-((2-hydroxyethyl)thio)tetrahydro-2H-pyran-4-yl)-4-methylbenzenesulfonamide

In a screw-cap vial, 2-mercaptoethanol (0.831 mL, 11.84 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 1.00 g, 1.974 mmol) and triethylamine (1.098 mL, 7.90 mmol) in methanol (5 mL). The vial was capped and stirred at 30° C. overnight. The mixture was partly concentrated, diluted with dichloromethane (3 mL), and filtered through a 0.45 μm nylon filter. The filtrate was purified by flash column chromatography (silica, 5 to 100% ethyl acetate in heptane) to give N-((2R,4S,5R)-2-((S)-1-(4-fluorophenyl)-1,2,3,4-tetrahydroisoquinoline-2-carbonyl)-5-((2-hydroxyethyl)thio)tetrahydro-2H-pyran-4-yl)-4-methylbenzenesulfonamide.

LCMS: 99%, RT=2.08 min., (M+H)⁺=585 (method A).

### Synthesis of ((S)-1-(4-fluorophenyl)-3,4-dihydroisoquinolin-2(1H)-yl)((4aR,7R,8aS)-1-tosyloctahydropyrano[3,4-b][1,4]thiazin-7-yl)methanone

In a 20 mL microwave vial under argon, cyanomethylenetributylphosphorane (0.421 mL, 1.603 mmol) was added to a solution of N-((2R,4S,5R)-2-((S)-1-(4-fluorophenyl)-1,2,3,4-tetrahydroisoquinoline-2-carbonyl)-5-((2-hydroxyethyl)thio)tetrahydro-2H-pyran-4-yl)-4-methylbenzenesulfonamide (625 mg, 1.069 mmol) in toluene (dry, 10 mL). The vial was capped and heated to 100° C. After 1.5 hours, the reaction mixture was concentrated to dryness under reduced pressure. The residue was purified by flash column chromatography (silica, 0 to 50% ethyl acetate in heptane) to give ((S)-1-(4-fluorophenyl)-3,4-dihydroisoquinolin-2(1H)-yl)((4aR,7R,8aS)-1-tosyloctahydropyrano[3,4-b][1,4]thiazin-7-yl)methanone.

LCMS: 89%, RT=2.30 min., (M+H)⁺=567 (method A).

### Synthesis of ((S)-1-(4-fluorophenyl)-3,4-dihydroisoquinolin-2(1H)-yl)((4aR,7R,8aS)-octahydropyrano[3,4-b][1,4]thiazin-7-yl)methanone (COMPOUND 5534)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.715 mL, 0.357 mmol) was added to a solution of ((S)-1-(4-fluorophenyl)-3,4-dihydroisoquinolin-2(1H)-yl)((4aR,7R,8aS)-1-tosyloctahydropyrano[3,4-b][1,4]thiazin-7-yl)methanone (135 mg, 0.238 mmol) in tetrahydrofuran (dry, 6.0 mL). After 15 minutes, the reaction mixture was allowed to warm to room temperature and stirred overnight. Then, the mixture was cooled again to −78° C. and in the course of 0.5 hour, additional sodium naphthalenide (0.5M in tetrahydrofuran, 2.3 mL, 1.125 mmol) was added until the color of the reagent (dark green) persisted. After 15 minutes, the mixture was quenched by addition of saturated aqueous NH₄Cl (10 mL), warmed to room temperature, and concentrated under reduced pressure to remove most of the tetrahydrofuran. The residual aqueous phase was extracted with dichloromethane (3×10 mL). The combined organics were passed through a hydrophobic frit and evaporated under reduced pressure. The residue was purified by flash column chromatography (silica, 0 to 5% (7M NH₃ in methanol) in dichloromethane) and preparative SFC (method AN) to give ((S)-1-(4-fluorophenyl)-3,4-dihydroisoquinolin-2(1H)-yl)((4aR,7R,8aS)-octahydropyrano[3,4-b][1,4]thiazin-7-yl)methanone (COMPOUND 5534) as the first eluting SFC isomer after lyophilization from a mixture of acetonitrile and water (1:1, 4 mL).

LCMS: 99%, RT=2.90 min., (M+H)⁺=413 (method AK). SFC: 100%, RT=3.76 min., (M+H)⁺=413 (method AD).

### Example 137

### ((S)-1-(4-fluorophenyl)-3,4-dihydroisoquinolin-2(1H)-yl)((4aR,7R,8aS)-3-(hydroxymethyl)octahydropyrano[3,4-b][1,4]oxazin-7-yl)methanone (COMPOUND 5535)

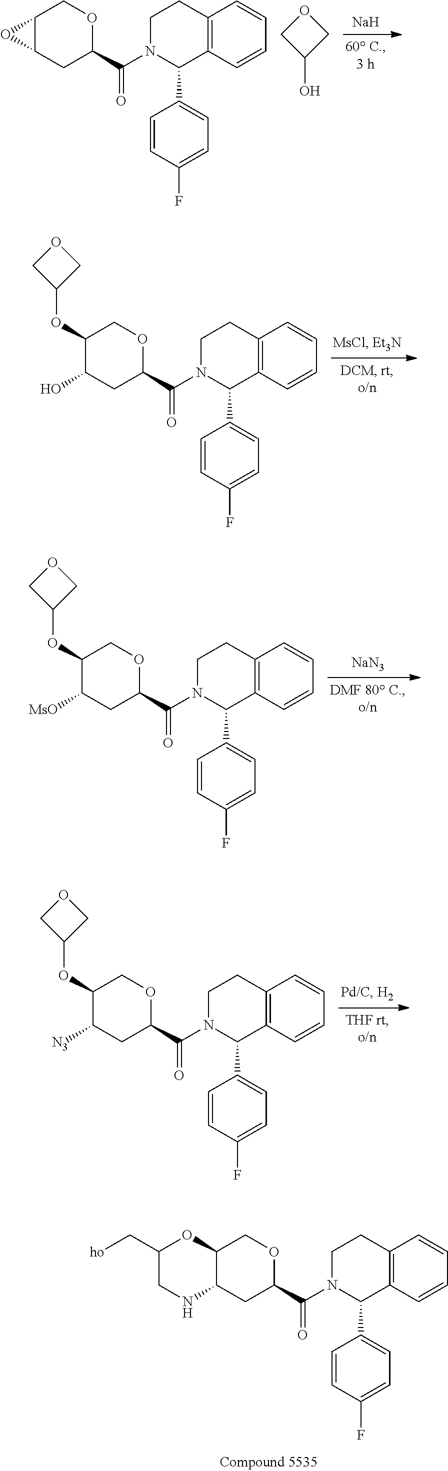

### Synthesis of ((S)-1-(4-fluorophenyl)-3,4-dihydroisoquinolin-2(1H)-yl)((2R,4S,5S)-4-hydroxy-5-(oxetan-3-yloxy)tetrahydro-2H-pyran-2-yl)methanone

3-Oxetanol (337 μL, 5.31 mmol) was added to sodium hydride (60% dispersion in mineral oil, 21.22 mg, 0.531 mmol). After 5 minutes, ((1R,4R,6S)-3,7-dioxabicyclo[4.1.0]heptan-4-yl)((S)-1-(4-fluorophenyl)-3,4-dihydroisoquinolin-2(1H)-yl)methanone (see Compound 5070, 75 mg, 0.212 mmol) was added and the reaction was stirred at 60° C. After 2 hours, the mixture was diluted with dichloromethane (1 mL) and stirring was continued for another hour. After cooling to room temperature, the mixture was diluted with dichloromethane (3 mL) and washed with saturated aqueous NH₄Cl (3 mL). The organic phase was passed through a hydrophobic frit and evaporated under reduced pressure. The residue was purified by flash column chromatography (silica, 0 to 100% ethyl acetate in heptane) to give ((S)-1-(4-fluorophenyl)-3,4-dihydroisoquinolin-2(1H)-yl)((2R,4S,5S)-4-hydroxy-5-(oxetan-3-yloxy)tetrahydro-2H-pyran-2-yl)methanone.

LCMS: 96%, RT=1.86 min., (M+H)⁺=428 (method A).

### Synthesis of (2R,4S,5S)-2-((S)-1-(4-fluorophenyl)-1,2,3,4-tetrahydroisoquinoline-2-carbonyl)-5-(oxetan-3-yloxy)tetrahydro-2H-pyran-4-yl methanesulfonate

Triethylamine (0.066 mL, 0.477 mmol) and methanesulfonyl chloride (0.018 mL, 0.239 mmol) were added to a solution of ((S)-1-(4-fluorophenyl)-3,4-dihydroisoquinolin-2(1H)-yl)((2R,4S,5S)-4-hydroxy-5-(oxetan-3-yloxy)tetrahydro-2H-pyran-2-yl)methanone (68 mg, 0.159 mmol) in dichloromethane (4 mL). After stirring overnight, additional triethylamine (0.022 mL, 0.159 mmol) and methanesulfonyl chloride (0.012 mL, 0.159 mmol) were added and stirring was continued for 0.5 hour. Then, the reaction mixture was washed with aqueous citric acid (0.5 M, 4 mL) and saturated aqueous NaHCO₃ (4 mL) and the organic phase was passed through a hydrophobic frit and evaporated under reduced pressure to give (2R,4S,5S)-2-((S)-1-(4-fluorophenyl)-1,2,3,4-tetrahydroisoquinoline-2-carbonyl)-5-(oxetan-3-yloxy)tetrahydro-2H-pyran-4-yl methanesulfonate.

LCMS: 97%, RT=2.01 min., (M+H)⁺=506 (method A).

### Synthesis of ((2R,4S,5R)-4-azido-5-(oxetan-3-yloxy)tetrahydro-2H-pyran-2-yl)((S)-1-(4-fluorophenyl)-3,4-dihydroisoquinolin-2(1H)-yl)methanone

Sodium azide (20.06 mg, 0.309 mmol) was added to a solution of (2R,4S,5S)-2-((S)-1-(4-fluorophenyl)-1,2,3,4-tetrahydroisoquinoline-2-carbonyl)-5-(oxetan-3-yloxy)tetrahydro-2H-pyran-4-yl methanesulfonate (78 mg, 0.154 mmol) in N,N-dimethylformamide (4 mL) and the reaction mixture was heated to 80° C. After stirring overnight, the mixture was cooled to room temperature and partitioned between water and ethyl acetate. The organic layers was separated and evaporated under reduced pressure. The residue was purified by flash column chromatography (silica, 0 to 100% ethyl acetate in heptane) to give ((2R,4S,5R)-4-azido-5-(oxetan-3-yloxy)tetrahydro-2H-pyran-2-yl)((S)-1-(4-fluorophenyl)-3,4-dihydroisoquinolin-2(1H)-yl)methanone.

LCMS: 99%, RT=2.09 min., (M+H)⁺=453 (method A).

### Synthesis of ((S)-1-(4-fluorophenyl)-3,4-dihydroisoquinolin-2(1H)-yl)((4aR,7R,8aS)-3-(hydroxymethyl)octahydropyrano[3,4-b][1,4]oxazin-7-yl)methanone (COMPOUND 5535)

A solution of ((2R,4S,5R)-4-azido-5-(oxetan-3-yloxy)tetrahydro-2H-pyran-2-yl)((S)-1-(4-fluorophenyl)-3,4-dihydroisoquinolin-2(1H)-yl)methanone (40 mg, 0.088 mmol) in tetrahydrofuran (4 mL) was hydrogenated in the presence of palladium (10 wt % on carbon, containing 50% water, 3.76 mg, 1.77 μmol) at atmospheric hydrogen pressure. After stirring overnight, the mixture was filtered over a 0.22 μm nylon filter and the filtrate was concentrated under reduced pressure. The residue was dissolved in a minimum amount of methanol, brought onto an SCX-2 column (1 g), and eluted with methanol (˜5 column volumes). Next, the column was eluted with ammonia in methanol (2 M). The basic fraction was concentrated to dryness under reduced pressure. The residue was purified by preparative SFC (method Z) and the first eluting product fractions on SFC were combined and lyophilized. The product was dissolved in a minimum amount of methanol, brought onto an SCX-2 column (1 g), and eluted with methanol (˜5 column volumes). Next, the column was eluted with ammonia in methanol (1 M). The basic fraction was concentrated to dryness under reduced and lyophilized from a mixture of acetonitrile and water (1:1, 4 mL) to give ((S)-1-(4-fluorophenyl)-3,4-dihydroisoquinolin-2(1H)-yl)((4aR,7R,8aS)-3-(hydroxymethyl)octahydropyrano[3,4-b][1,4]oxazin-7-yl)methanone (COMPOUND 5535).

LCMS: 97%, RT=1.05 min., (M+H)⁺=427 (method P).

### Example 138

### ((4aR,7R,8aS)-4,4-dioxidooctahydropyrano[3,4-b][1,4]thiazin-7-yl)((S)-1-(4-fluorophenyl)-3,4-dihydroisoquinolin-2(1H)-yl)methanone (COMPOUND 5542)

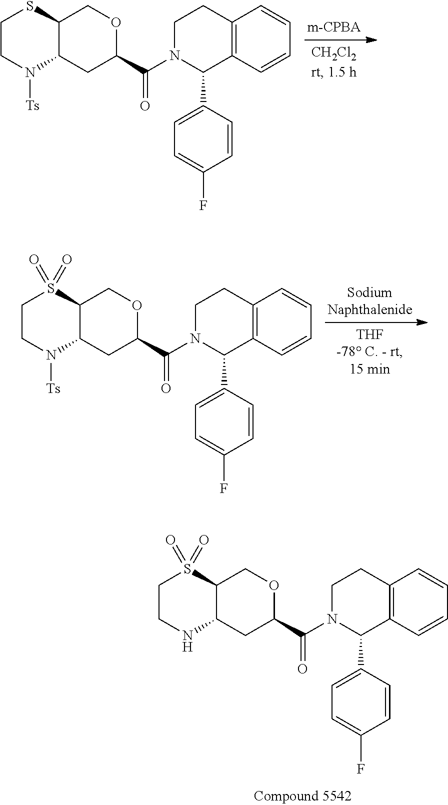 [SMILES: Cc1ccc(S(=O)(=O)N2CCS[C@H]3CO[C@@H](C(=O)N4CCc5ccccc5[C@@H]4c4ccc(F)cc4)C[C@@H]32)cc1 ; Cc1ccc(S(=O)(=O)N2CCS(=O)(=O)[C@H]3CO[C@@H](C(=O)N4CCc5ccccc5[C@@H]4c4ccc(F)cc4)C[C@@H]32)cc1 ; O=C([C@H]1C[C@@H]2NCCS(=O)(=O)[C@H]2CO1)N1CCc2ccccc2[C@@H]1c1ccc(F)cc1]

### Synthesis of ((4aR,7R,8aS)-4,4-dioxido-1-tosyloctahydropyrano[3,4-b][1,4]thiazin-7-yl)((S)-1-(4-fluorophenyl)-3,4-dihydroisoquinolin-2(1H)-yl)methanone

m-Chloroperbenzoic acid (70%, 26.1 mg, 0.106 mmol) was added to a solution of ((S)-1-(4-fluorophenyl)-3,4-dihydroisoquinolin-2(1H)-yl)((4aR,7R,8aS)-1-tosyloctahydropyrano[3,4-b][1,4]thiazin-7-yl)methanone (see Compound 5534, 60 mg, 0.106 mmol) in dichloromethane (dry, 2.0 mL). After respectively 0.5 hour and 1 hour, two additional portions of m-chloroperbenzoic acid (70%, 2 times 19.58 mg, 0.079 mmol) were added. After a total reaction time of 1.5 hour, the mixture was diluted with dichloromethane (10 mL) and saturated aqueous Na₂S₂O₃ (10 mL) and stirred vigorously for 15 minutes. The layers were separated and the aqueous phase was extracted with dichloromethane (10 mL). The combined organics were dried on Na₂SO₄ and evaporated under reduced pressure to give ((4aR,7R,8aS)-4,4-dioxido-1-tosyloctahydropyrano[3,4-b][1,4]thiazin-7-yl)((S)-1-(4-fluorophenyl)-3,4-dihydroisoquinolin-2(1H)-yl)methanone (55.0 mg) as a pale wax.

LCMS: 93%, RT=2.14 min., (M+H)⁺=599 (method A).

### Synthesis of ((4aR,7R,8aS)-4,4-dioxidooctahydropyrano[3,4-b][1,4]thiazin-7-yl)((S)-1-(4-fluorophenyl)-3,4-dihydroisoquinolin-2(1H)-yl)methanone (COMPOUND 5542)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.735 mL, 0.367 mmol) was added to a solution of ((4aR,7R,8aS)-4,4-dioxido-1-tosyloctahydropyrano[3,4-b][1,4]thiazin-7-yl)((S)-1-(4-fluorophenyl)-3,4-dihydroisoquinolin-2(1H)-yl)methanone (55 mg, 0.092 mmol) in tetrahydrofuran (dry, 2.5 mL). After 15 minutes, the reaction mixture was quenched by addition of methanol (0.4 mL) and stored in the freezer overnight. Then, the mixture was concentrated to dryness under reduced pressure. The residue was purified by flash column chromatography (silica, 0 to 6% (7M NH₃ in methanol) in dichloromethane) and acidic preparative MPLC (Linear Gradient: t=0 min 5% A; t=1 min 5% A; t=2 min 10% A; t=17 min 50% A; t=18 min 100%; t=23 min 100% A; detection: 220 nm). The product fractions of the acidic preparative MPLC were combined, basified with saturated aqueous Na₂CO₃ (2 mL) and extracted with dichloromethane (3×10 mL). The combined organics were dried over Na₂SO₄ and evaporated under reduced pressure. The residue was lyophilized from a mixture of acetonitrile and water (1:1, 4 mL) to give ((4aR,7R,8aS)-4,4-dioxidooctahydropyrano[3,4-b][1,4]thiazin-7-yl)((S)-1-(4-fluorophenyl)-3,4-dihydroisoquinolin-2(1H)-yl)methanone (COMPOUND 5542).

LCMS: 94%, RT=1.21 min., (M+H)⁺=445 (method P).

### Example 139

### ((S)-1-(4-fluorophenyl)-3,4-dihydroisoquinolin-2(1H)-yl)((3R,4aR,7R,8aS)-3-methyloctahydropyrano[3,4-b][1,4]oxazin-7-yl)methanone (COMPOUND 5472)

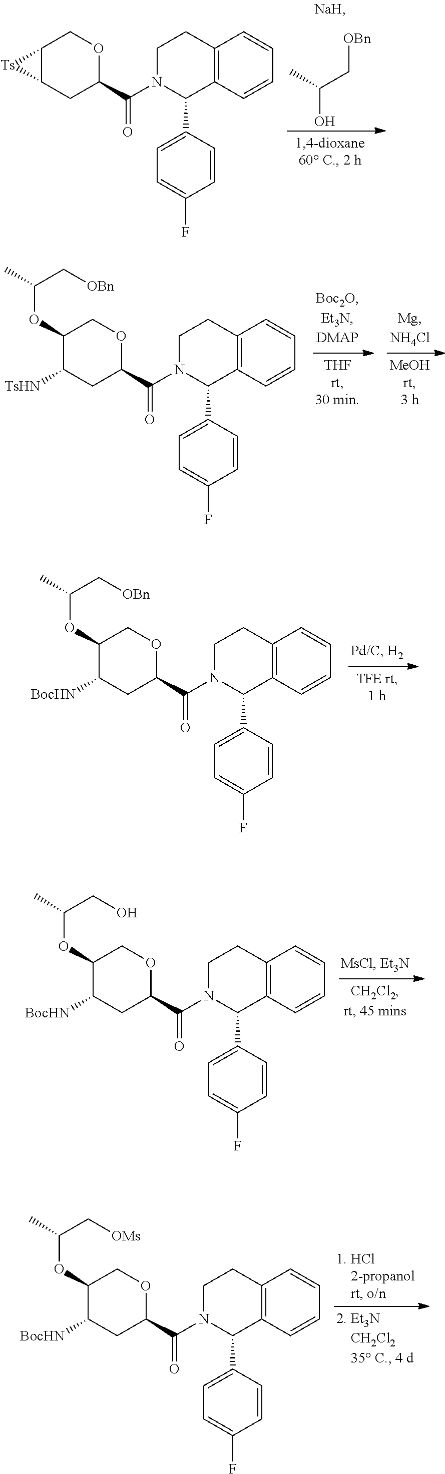

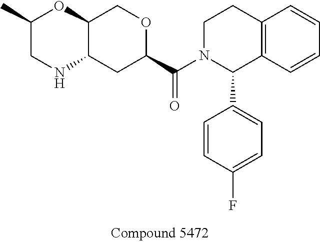 [SMILES: C[C@@H]1CN[C@H]2C[C@H](C(=O)N3CCc4ccccc4[C@@H]3c3ccc(F)cc3)OC[C@@H]2O1]

### Synthesis of N-((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide

Under an argon atmosphere, sodium hydride (60% dispersion in mineral oil, 42.6 mg, 1.066 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 360 mg, 0.711 mmol) and (R)-(−)-1-benzyloxy-2-propanol (591 mg, 3.55 mmol) in 1,4-dioxane (10 mL) and the mixture was heated to 60° C. After 2 hours, the mixture was cooled to room temperature, diluted with half saturated aqueous NH₄Cl, and extracted with ethyl acetate (2×25 mL). The combined organics were washed with brine, dried over Na₂SO₄ and evaporated under reduced pressure. The residue was purified by flash column chromatography (silica, 5 to 60% ethyl acetate in heptane) to give N-((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 97%, RT=2.56 min., (M+H)⁺=673 (method A).

### Synthesis of tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.097 mL, 0.699 mmol), di-tert-butyl dicarbonate (0.162 mL, 0.699 mmol), and 4-dimethylaminopyridine (4.27 mg, 0.035 mmol) were added to a solution of N-((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide (235 mg, 0.349 mmol) in tetrahydrofuran (dry, 4 mL). After 30 minutes, the mixture was concentrated under reduced pressure. The residue was purified by flash column chromatography (silica, 5 to 40% ethyl acetate in heptane) to give tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate after co-evaporation from dichloromethane.

LCMS: 89%, RT=2.58 min., (M+H)⁺=773 (method B).

### Synthesis of tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate

Ammonium chloride (163 mg, 3.04 mmol) and magnesium (369 mg, 15.20 mmol) were added to a solution of tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (235 mg, 0.304 mmol) in methanol. After 3 hours, the mixture was diluted with saturated aqueous NH₄Cl (50 mL) and dichloromethane and stirred for 10 minutes. The layers were separated and the aqueous layer was extracted with dichloromethane. The combined organics were dried over Na₂SO₄ and evaporated under reduced pressure. The residue was purified by flash column chromatography (silica, 5 to 50% ethyl acetate in heptane) to give tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate after co-evaporation from dichloromethane.

LCMS: 97%, RT=2.34 min., (M+Na)⁺=641 (method A).

### Synthesis of tert-butyl ((2R,4S,5R)-2-((S)-1-(4-fluorophenyl)-1,2,3,4-tetrahydroisoquinoline-2-carbonyl)-5-(((R)-1-hydroxypropan-2-yl)oxy)tetrahydro-2H-pyran-4-yl)carbamate

A solution of tert-butyl ((2R,4S,5R)-5-(((R)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate (145 mg, 0.234 mmol) in 2,2,2-trifluoroethanol (3 mL) was hydrogenated in the presence of palladium (10 wt % on carbon, containing 50% water, 60 mg, 0.028 mmol) at atmospheric hydrogen pressure. After stirring for 1 hour, the mixture was filtered over a 0.45 μm nylon filter. The filtrate was concentrated under reduced pressure to give tert-butyl ((2R,4S,5R)-2-((S)-1-(4-fluorophenyl)-1,2,3,4-tetrahydroisoquinoline-2-carbonyl)-5-(((R)-1-hydroxypropan-2-yl)oxy)tetrahydro-2H-pyran-4-yl)carbamate.

### Synthesis of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propy methanesulfonate

Triethylamine (0.093 mL, 0.664 mmol) and methanesulfonyl chloride (0.034 mL, 0.443 mmol) were added to a solution of tert-butyl ((2R,4S,5R)-2-((S)-1-(4-fluorophenyl)-1,2,3,4-tetrahydroisoquinoline-2-carbonyl)-5-(((R)-1-hydroxypropan-2-yl)oxy)tetrahydro-2H-pyran-4-yl)carbamate (117 mg, 0.221 mmol) in dichloromethane. After 45 minutes, the mixture was diluted with saturated aqueous NaHCO₃ (4 mL) and stirred for 15 minutes. The layers were separated over a phase separation filter and the organic filtrate was evaporated under reduced pressure to (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propyl methanesulfonate.

LCMS: 97%, RT=2.14 min., (M+H)⁺=607 (method A).

### Synthesis of ((S)-1-(4-fluorophenyl)-3,4-dihydroisoquinolin-2(1H)-yl)((3R,4aR,7R,8aS)-3-methyloctahydropyrano[3,4-b][1,4]oxazin-7-yl)methanone (COMPOUND 5472)

HCl (5-6 M in 2-propanol, 1 mL, 5.50 mmol) was added to a solution of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propyl methanesulfonate (136 mg, 0.224 mmol) in dichloromethane. After stirring overnight, the mixture was diluted with saturated aqueous NaHCO₃ and dichloromethane and stirred vigorously for 15 minutes. The layers were separated over a phase separation filter and the organic filtrate was evaporated under reduced pressure. The residue was dissolved in dichloromethane and triethylamine (0.1 mL, 0.717 mmol) was added. After stirring for 1.5 hour, the mixture was warmed to 35° C. and stirred for 4 days. Then, the mixture was concentrated under reduced pressure. The residue was dissolved in methanol, brought onto an SCX-2 column (1 g), and eluted with methanol (5 column volumes). Next, the column was eluted with ammonia in methanol (2 M). The basic fraction was concentrated to dryness under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product containing fractions were combined, neutralised with saturated aqueous NaHCO₃, and extracted with dichloromethane twice. The combined organics were dried over Na₂SO₄ and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((3R,4aR,7R,8aS)-3-methyloctahydropyrano[3,4-b][1,4]oxazin-7-yl)methanone (COMPOUND 5472)

LCMS: 100%, RT=1.14 min., (M+H)⁺=411 (method P).

### Example 140

### (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)-⁶-sulfanone (COMPOUND 5547) and (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)-⁶-sulfanone (COMPOUND 5548)

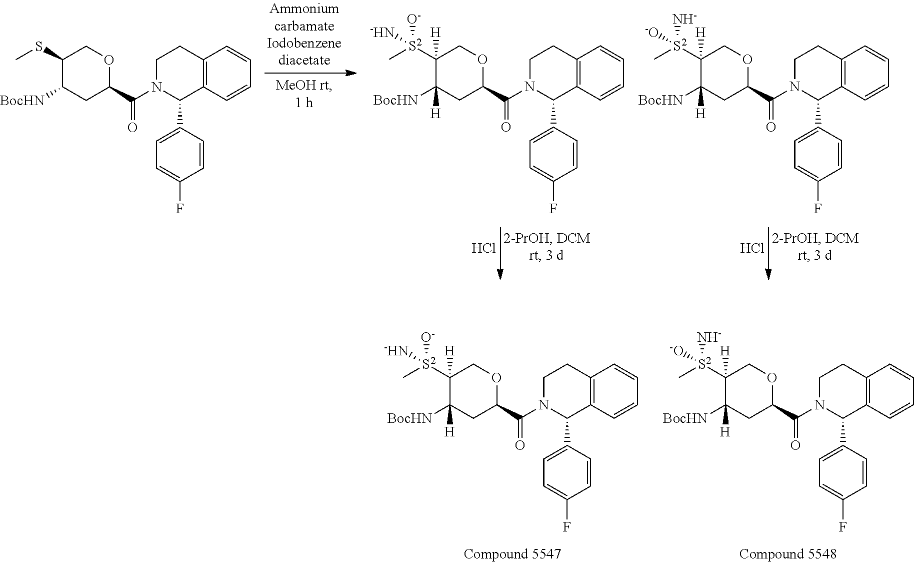

### Synthesis of tert-butyl ((2R,4S,5R)-2-((S)-1-(4-fluorophenyl)-1,2,3,4-tetrahydroisoquinoline-2-carbonyl)-5-((R)-S-methylsulfonimidoyl)tetrahydro-2H-pyran-4-yl)carbamate and tert-butyl ((2R,4S,5R)-2-((S)-1-(4-fluorophenyl)-1,2,3,4-tetrahydroisoquinoline-2-carbonyl)-5-((S)-S-methylsulfonimidoyl)tetrahydro-2H-pyran-4-yl)carbamate

Iodobenzene diacetate (438 mg, 1.359 mmol) was added to a suspension of tert-butyl ((2R,4S,5R)-2-((S)-1-(4-fluorophenyl)-1,2,3,4-tetrahydroisoquinoline-2-carbonyl)-5-(methylthio)tetrahydro-2H-pyran-4-yl)carbamate (see Compound 5520, 324 mg, 0.647 mmol) and ammonium carbamate (76 mg, 0.971 mmol) in methanol (4 mL). The reaction mixture was stirred under air and after 1 hour, the mixture was concentrated under reduced pressure. The residue was purified by flash column chromatography (silica, 0 to 5% (7M ammonia in methanol) in dichloromethane) and preparative SFC (method BX) to give tert-butyl ((2R,4S,5R)-2-((S)-1-(4-fluorophenyl)-1,2,3,4-tetrahydroisoquinoline-2-carbonyl)-5-((R)-S-methylsulfonimidoyl)tetrahydro-2H-pyran-4-yl)carbamate as the first eluting SFC isomer (iAmylose column) and tert-butyl ((2R,4S,5R)-2-((S)-1-(4-fluorophenyl)-1,2,3,4-tetrahydroisoquinoline-2-carbonyl)-5-((S)-S-methylsulfonimidoyl)tetrahydro-2H-pyran-4-yl)carbamate as the second eluting SFC isomer (iAmylose column). Stereochemistry of sulfoximine stereocenter is arbitrarily assigned.

First eluting isomer: LCMS: 94%, RT=1.96 min., (M+H)⁺=532 (method A). SFC: 94%, RT=2.81 min., (M+H)⁺=532 (method V).

Second eluting isomer: LCMS: 93%, RT=1.95 min., (M+H)⁺=532 (method A). SFC: 100%, RT=2.67 min., (M+H)⁺=532 (method V).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)-⁶-sulfanone (COMPOUND 5547)

HCl (5-6 M in 2-propanol, 0.5 mL, 2.75 mmol) was added to a suspension of tert-butyl ((2R,4S,5R)-2-((S)-1-(4-fluorophenyl)-1,2,3,4-tetrahydroisoquinoline-2-carbonyl)-5-((R)-S-methylsulfonimidoyl)tetrahydro-2H-pyran-4-yl)carbamate (50 mg, 0.094 mmol) in 2-propanol (2 mL) and dichloromethane (0.5 mL). After stirring for 3 days, the reaction mixture was diluted with dichloromethane (5 mL) and neutralized with saturated aqueous NaHCO₃. The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:1, 3 mL) to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)- [symbol] ⁶-sulfanone (COMPOUND 5547). Stereochemistry of sulfoximine stereocenter is arbitrarily assigned.

LCMS: 95%, RT=2.55 min., (M+H)⁺=432 (method AK). SFC: 95%, RT=3.92 min., (M+H)⁺=432 (method W).

### Synthesis of (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)-⁶-sulfanone (COMPOUND 5548)

HCl (5-6 M in 2-propanol, 0.5 mL, 2.75 mmol) was added to a suspension of tert-butyl ((2R,4S,5R)-2-((S)-1-(4-fluorophenyl)-1,2,3,4-tetrahydroisoquinoline-2-carbonyl)-5-((S)-S-methylsulfonimidoyl)tetrahydro-2H-pyran-4-yl)carbamate (40 mg, 0.075 mmol) in 2-propanol (2 mL) and dichloromethane (0.5 mL). After stirring for 3 days, the reaction mixture was diluted with dichloromethane (5 mL) and neutralized with saturated aqueous NaHCO₃. The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:1, 3 mL) to give (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(methyl)- [symbol] ⁶-sulfanone (COMPOUND 5548). Stereochemistry of sulfoximine stereocenter is arbitrarily assigned.

LCMS: 93%, RT=2.58 min., (M+H)⁺=432 (method AK). SFC: 99%, RT=4.43 min., (M+H)⁺=432 (method W).

### Example 141

### (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)-⁶-sulfanone (COMPOUND 5549) and (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)-⁶-sulfanone (COMPOUND 5550)

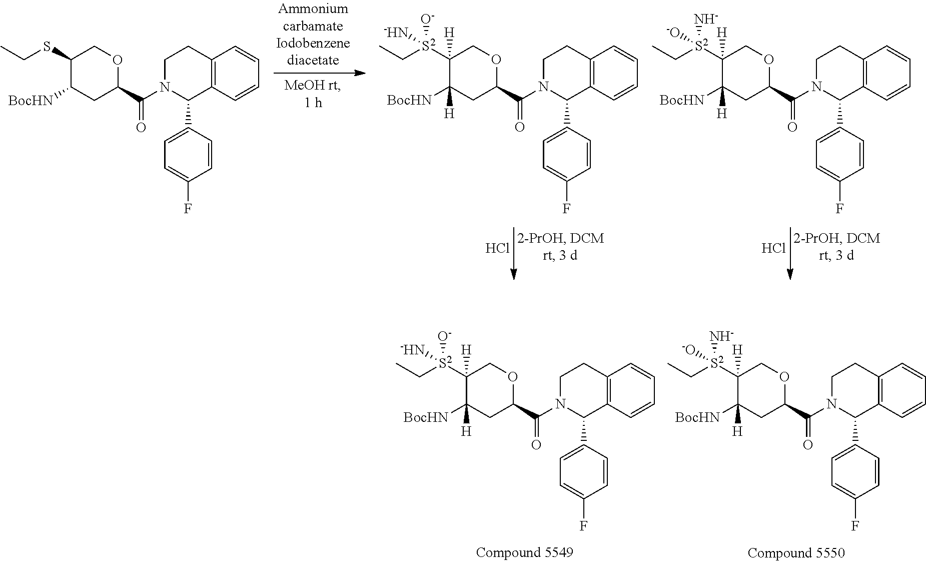

### Synthesis of tert-butyl ((2R,4S,5R)-5-((R)-ethylsulfonimidoyl)-2-((S)-1-(4-fluorophenyl)-1,2,3,4-tetrahydroisoquinoline-2-carbonyl)tetrahydro-2H-pyran-4-yl)carbamate and tert-butyl ((2R,4S,5R)-5-((S)-ethylsulfonimidoyl)-2-((S)-1-(4-fluorophenyl)-1,2,3,4-tetrahydroisoquinoline-2-carbonyl)tetrahydro-2H-pyran-4-yl)carbamate

Iodobenzene diacetate (668 mg, 2.073 mmol) was added to a suspension of tert-butyl ((2R,4S,5R)-5-(ethylthio)-2-((S)-1-(4-fluorophenyl)-1,2,3,4-tetrahydroisoquinoline-2-carbonyl)tetrahydro-2H-pyran-4-yl)carbamate (508 mg, 0.987 mmol) (see Compound 5521, 508 mg, 0.987 mmol) and ammonium carbamate (116 mg, 1.481 mmol) in methanol (6 mL). The reaction mixture was stirred under air and after 1 hour, the mixture was concentrated under reduced pressure. The residue was purified by flash column chromatography (silica, 0 to 10% (7M ammonia in methanol) in dichloromethane) and preparative SFC (method BY) to give tert-butyl ((2R,4S,5R)-5-((R)-ethylsulfonimidoyl)-2-((S)-1-(4-fluorophenyl)-1,2,3,4-tetrahydroisoquinoline-2-carbonyl)tetrahydro-2H-pyran-4-yl)carbamate as the first eluting SFC isomer and tert-butyl ((2R,4S,5R)-5-((S)-ethylsulfonimidoyl)-2-((S)-1-(4-fluorophenyl)-1,2,3,4-tetrahydroisoquinoline-2-carbonyl)tetrahydro-2H-pyran-4-yl)carbamate as the second eluting SFC isomer. Stereochemistry of the sulfoximine stereocenter is arbitrarily assigned.

First eluting isomer: LCMS: 98%, RT=1.68 min., (M+H)⁺=546 (method P). SFC: 99%, RT=4.99 min., (M+H)⁺=546 (method BZ).

Second eluting isomer: LCMS: 96%, RT=1.68 min., (M+H)⁺=546 (method A). SFC: 95%, RT=5.34 min., (M+H)⁺=546 (method BZ).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)-⁶-sulfanone (COMPOUND 5549)

HCl (5-6 M in 2-propanol, 1 mL, 5.5 mmol) was added to a suspension of tert-butyl ((2R,4S,5R)-5-((R)-ethylsulfonimidoyl)-2-((S)-1-(4-fluorophenyl)-1,2,3,4-tetrahydroisoquinoline-2-carbonyl)tetrahydro-2H-pyran-4-yl)carbamate (80 mg, 0.147 mmol) in 2-propanol (3 mL) and dichloromethane (1 mL). After stirring overnight, another portion of HCl (5-6 M in 2-propanol, 0.5 mL, 2.75 mmol) was added and stirring was continued for 2 hours. The reaction mixture was diluted with dichloromethane (5 mL) and neutralized with saturated aqueous NaHCO₃. The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:1, 3 mL). The product still contained some starting material and was resubmitted and purified according the procedure described above to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)- [symbol] ⁶-sulfanone (COMPOUND 5549). Stereochemistry of the sulfoximine stereocenter is arbitrarily assigned.

LCMS: 97%, RT=2.61 min., (M+H)⁺=446 (method AK). SFC: 99%, RT=3.94 min., (M+H)⁺=446 (method W).

### Synthesis of (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)-⁶-sulfanone (COMPOUND 5550)

HCl (5-6 M in 2-propanol, 1 mL, 5.5 mmol) was added to a suspension of tert-butyl ((2R,4S,5R)-5-((S)-ethylsulfonimidoyl)-2-((S)-1-(4-fluorophenyl)-1,2,3,4-tetrahydroisoquinoline-2-carbonyl)tetrahydro-2H-pyran-4-yl)carbamate (80 mg, 0.147 mmol) in 2-propanol (3 mL) and dichloromethane (1 mL). After stirring overnight, another portion of HCl (5-6 M in 2-propanol, 0.5 mL, 2.75 mmol) was added and stirring was continued for 2 hours. The reaction mixture was diluted with dichloromethane (5 mL) and neutralized with saturated aqueous NaHCO₃. The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:1, 3 mL). The product still contained some starting material and was resubmitted and purified according the procedure described above to give (S)-((3R,4S,6R)-4-amino-6-((S)-1-(4-fluorophenyl)-1,2,3,4-tetrahydroisoquinoline-2-carbonyl)tetrahydro-2H-pyran-3-yl)(imino)(ethyl)- [symbol] ⁶-sulfanone (COMPOUND 5550). Stereochemistry of the sulfoximine stereocenter is arbitrarily assigned.

LCMS: 94%, RT=1.38 min., (M+H)⁺=446 (method Q). SFC: 96%, RT=4.37 min., (M+H)⁺=446 (method W).

### Example 142

### (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)-⁶-sulfanone (COMPOUND 5551)

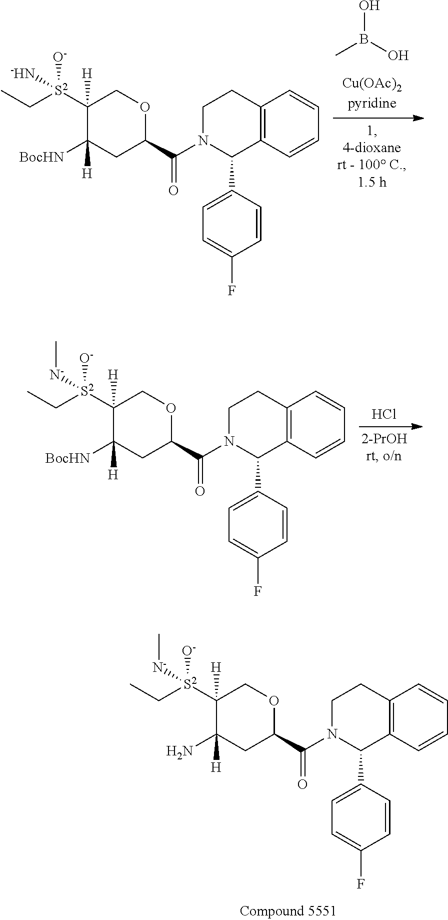

### Synthesis of tert-butyl ((2R,4S,5R)-2-((S)-1-(4-fluorophenyl)-1,2,3,4-tetrahydroisoquinoline-2-carbonyl)-5-((R)-N-methylethylsulfonimidoyl)tetrahydro-2H-pyran-4-yl)carbamate

Molecular sieves (4 Å) were added to a solution of tert-butyl ((2R,4S,5R)-5-((R)-ethylsulfonimidoyl)-2-((S)-1-(4-fluorophenyl)-1,2,3,4-tetrahydroisoquinoline-2-carbonyl)tetrahydro-2H-pyran-4-yl)carbamate (see COMPOUND 5549, 58 mg, 0.106 mmol), copper (II) acetate (29.0 mg, 0.159 mmol) and pyridine (0.021 mL, 0.255 mmol) in 1,4-dioxane (1 mL). The reaction vessel was closed and stirred for 5 minutes. Then methylboronic acid (12.72 mg, 0.213 mmol) was added and the reaction mixture was stirred for 5 minutes under air. The reaction vessel was sealed and heated to 100° C. After 1.5 hour, the reaction mixture was cooled to room temperature, diluted with dichloromethane (4 mL), and washed with water (4 mL). The organic layer was passed through a hydrophobic frit and the filtrate was evaporated under reduced pressure. The residue was purified by flash column chromatography twice (silica, 0 to 100% ethyl acetate in heptane) and (silica, 0 to 70% ethyl acetate in heptane) to give tert-butyl ((2R,4S,5R)-2-((S)-1-(4-fluorophenyl)-1,2,3,4-tetrahydroisoquinoline-2-carbonyl)-5-((R)-N-methylethylsulfonimidoyl)tetrahydro-2H-pyran-4-yl)carbamate. Stereochemistry of the sulfoximine stereocenter is arbitrarily assigned.

LCMS: 99%, RT=1.97 min., (M+H)⁺=560 (method A).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)-⁶-sulfanone (COMPOUND 5551)

HCl (5-6 M in 2-propanol, 2 mL, 11.0 mmol) was added to a solution of tert-butyl ((2R,4S,5R)-2-((S)-1-(4-fluorophenyl)-1,2,3,4-tetrahydroisoquinoline-2-carbonyl)-5-((R)-N-methylethylsulfonimidoyl)tetrahydro-2H-pyran-4-yl)carbamate (50 mg, 0.089 mmol) in 2-propanol (4 mL). After stirring overnight, the reaction mixture was diluted with dichloromethane (15 mL) and neutralised with saturated aqueous NaHCO₃. The layers were separated using a phase-separator and the organic filtrate was evaporated under reduced pressure. The residue was dissolved in methanol (2 mL) and brought onto an SCX-2 column (2 g) and eluted with methanol until neutral. Next, the column was eluted with ammonia in methanol (1.5 M). The basic fraction was concentrated to dryness under reduced pressure. The residue was lyophilized from a mixture of acetonitrile and water (1:1, 3 mL) to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)- [symbol] [symbol] ⁶-sulfanone (COMPOUND 5551). Stereochemistry of the sulfoximine stereocenter is arbitrarily assigned.

LCMS: 97%, RT=1.48 min., (M+H)⁺=460 (method P). SFC: 99%, RT=4.17 min., (M+H)⁺=460 (method AD).

### Example 143

### (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)-⁶-sulfanone (COMPOUND 5552)

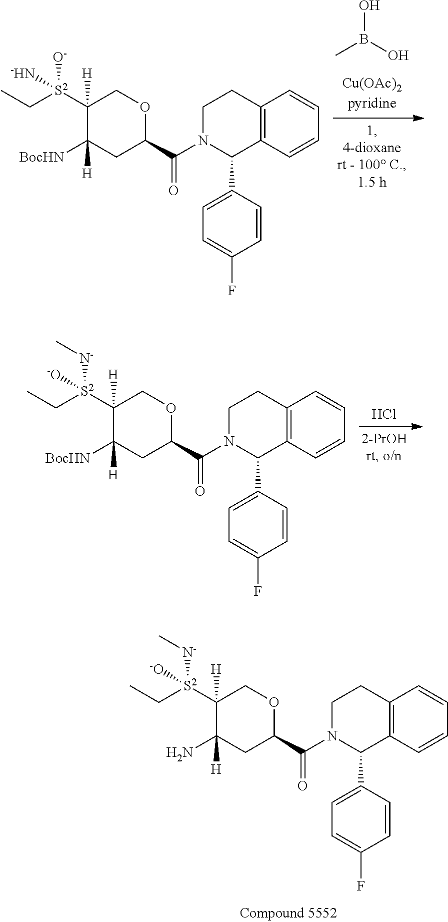

### Synthesis of tert-butyl ((2R,4S,5R)-2-((S)-1-(4-fluorophenyl)-1,2,3,4-tetrahydroisoquinoline-2-carbonyl)-5-((S)-N-methylethylsulfonimidoyl)tetrahydro-2H-pyran-4-yl)carbamate

Molecular sieves (4 Å) were added to a solution of tert-butyl ((2R,4S,5R)-5-((S)-ethylsulfonimidoyl)-2-((S)-1-(4-fluorophenyl)-1,2,3,4-tetrahydroisoquinoline-2-carbonyl)tetrahydro-2H-pyran-4-yl)carbamate (see COMPOUND 5550, 68 mg, 0.125 mmol), copper (II) acetate (34.0 mg, 0.187 mmol) and pyridine (0.024 mL, 0.299 mmol) in 1,4-dioxane (1.2 mL). The reaction vessel was closed and stirred for 5 minutes. Then methylboronic acid (12.72 mg, 0.213 mmol) was added and the reaction mixture was stirred for 5 minutes under air. The reaction vessel was sealed and heated to 100° C. After 1.5 hour, the reaction mixture was cooled to room temperature, diluted with dichloromethane (10 mL), and washed with water (5 mL). The organic layers was passed through a hydrophobic frit and the filtrate was evaporated under reduced pressure. The residue was purified by flash column chromatography twice (silica, 0 to 100% ethyl acetate in heptane) and (silica, 0 to 70% ethyl acetate in heptane) to give tert-butyl ((2R,4S,5R)-2-((S)-1-(4-fluorophenyl)-1,2,3,4-tetrahydroisoquinoline-2-carbonyl)-5-((S)-N-methylethylsulfonimidoyl)tetrahydro-2H-pyran-4-yl)carbamate. Stereochemistry of the sulfoximine stereocenter is arbitrarily assigned.

LCMS: 97%, RT=1.96 min., (M+H)⁺=560 (method A).

### Synthesis of (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)-⁶-sulfanone (COMPOUND 5552)

HCl (5-6 M in 2-propanol, 1 mL, 5.5 mmol) was added to a solution of tert-butyl ((2R,4S,5R)-2-((S)-1-(4-fluorophenyl)-1,2,3,4-tetrahydroisoquinoline-2-carbonyl)-5-((S)-N-methylethylsulfonimidoyl)tetrahydro-2H-pyran-4-yl)carbamate (33 mg, 0.059 mmol) in 2-propanol (3 mL). After stirring for 3 days, the reaction mixture was diluted with dichloromethane and neutralised with saturated aqueous NaHCO₃. The layers were separated using a phase-separator and the organic filtrate was evaporated under reduced pressure. The residue was dissolved in methanol (2 mL) and brought onto an SCX-2 column (1 g) and eluted with methanol until neutral. Next, the column was eluted with ammonia in methanol (2 M). The basic fraction was concentrated to dryness under reduced pressure. The residue was lyophilized from a mixture of acetonitrile and water (1:1, 4 mL) to give (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)(methylimino)- [symbol] ⁶-sulfanone (COMPOUND 5552). Stereochemistry of the sulfoximine stereocenter is arbitrarily assigned.

LCMS: 98%, RT=1.09 min., (M+H)⁺=460 (method P). SFC: 95%, RT=4.36 min., (M+H)⁺=460 (method AD).

### Example 144

### (4aR,7R,8aS)-4-ethyl-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5536)

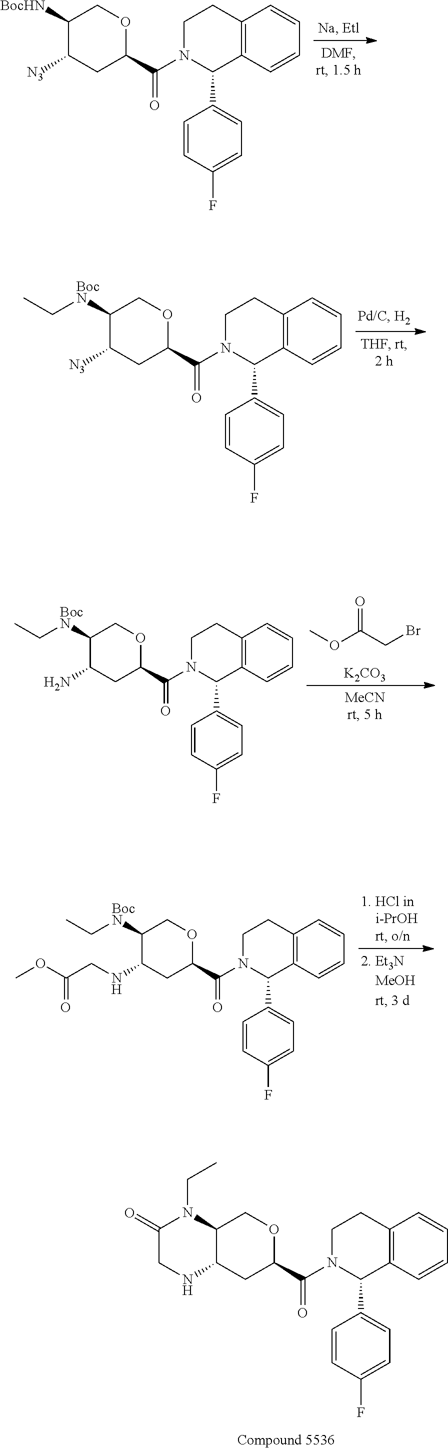

### Synthesis of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(ethyl)carbamate

Under an argon atmosphere, sodium hydride (60% in mineral oil, 21 mg, 0.525 mmol) was added to a solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)carbamate (see Compound 5532, 200 mg, 0.371 mmol) in N,N-dimethylformamide (2 mL). After stirring for 10 minutes, iodoethane (0.090 mL, 1.114 mmol) was added and stirring was continued for 1.5 hour. Then, the mixture was diluted with water and ethyl acetate. The layers were separated and the aqueous phase was extracted with ethyl acetate. The combined organic layers were washed with brine twice, dried over Na₂SO₄ and evaporated under reduced pressure. The residue was purified by flash column chromatography (silica, 5 to 40% ethyl acetate in heptane) to give tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(ethyl)carbamate after co-evaporation from dichloromethane.

LCMS: 99%, RT=2.34 min., (M+H)⁺=524 (method A).

### Synthesis of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)carbamate

A solution of tert-butyl ((3R,4S,6R)-4-azido-6-((S)-1-(4-fluorophenyl)-1,2,3,4-tetrahydroisoquinoline-2-carbonyl)tetrahydro-2H-pyran-3-yl)(ethyl)carbamate (165 mg, 0.315 mmol) in tetrahydrofuran (4 mL) was hydrogenated in the presence of palladium (10 wt % on carbon, containing 50% water, 80 mg, 0.038 mmol) at atmospheric hydrogen pressure. After stirring for 2 hours, the mixture was filtered over a 0.45 μm nylon filter. The filtrate was concentrated under reduced pressure to give tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)carbamate.

LCMS: 97%, RT=1.79 min., (M+H)⁺=498 (method A).

### Synthesis of methyl ((2R,4S,5R)-5-((tert-butoxycarbonyl)(ethyl)amino)-2-((S)-1-(4-fluorophenyl)-1,2,3,4-tetrahydroisoquinoline-2-carbonyl)tetrahydro-2H-pyran-4-yl)glycinate

Methyl bromoacetate (0.044 mL, 0.467 mmol) was added to a suspension of tert-butyl ((3R,4S,6R)-4-amino-6-((S)-1-(4-fluorophenyl)-1,2,3,4-tetrahydroisoquinoline-2-carbonyl)tetrahydro-2H-pyran-3-yl)(ethyl)carbamate (155 mg, 0.311 mmol) and potassium carbonate (108 mg, 0.779 mmol) in acetonitril (2.5 mL). After stirring for 5 hours, the mixture was diluted with water and dichloromethane. The layers were separated over a phase separation filter and the filtrate was concentrated under reduced pressure. The residue was purified by flash column chromatography (silica, 10 to 100% ethyl acetate in heptane) to give methyl ((2R,4S,5R)-5-((tert-butoxycarbonyl)(ethyl)amino)-2-((S)-1-(4-fluorophenyl)-1,2,3,4-tetrahydroisoquinoline-2-carbonyl)tetrahydro-2H-pyran-4-yl)glycinate after co-evaporation from dichloromethane.

LCMS: 99%, RT=1.86 min., (M+H)⁺=570 (method A).

### Synthesis of (4aR,7R,8aS)-4-ethyl-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5536)

HCl (6 M in 2-propanol, 1 mL, 6.00 mmol) was added to a solution of methyl ((2R,4S,5R)-5-((tert-butoxycarbonyl)(ethyl)amino)-2-((S)-1-(4-fluorophenyl)-1,2,3,4-tetrahydroisoquinoline-2-carbonyl)tetrahydro-2H-pyran-4-yl)glycinate (135 mg, 0.237 mmol) in 2-propanol (3 mL). After stirring overnight, the reaction mixture was diluted with dichloromethane and saturated aqueous NaHCO₃ and the mixture was stirred vigorously for 15 minutes. The layers were separated using a phase-separator and the organic filtrate was evaporated under reduced pressure. The residue was dissolved in methanol (3 mL) and triethylamine (0.066 mL, 0.474 mmol) was added. After 1 hour, another portion of triethylamine (0.033 mL, 0.237 mmol) was added and stirring was continued for 3 days. Then, the mixture was concentrated under reduced pressure. The residue was purified by flash column chromatography (silica, 1 to 10% (7 M NH₃ in methanol) in dichloromethane) to give (4aR,7R,8aS)-4-ethyl-7-((S)-1-(4-fluorophenyl)-1,2,3,4-tetrahydroisoquinoline-2-carbonyl)hexahydro-2H-pyrano[3,4-b]pyrazin-3(4H)-one (COMPOUND 5536) after lyophilization from a mixture of acetonitrile and water (1:1, 4 mL).

LCMS: 99%, RT=1.11 min., (M+H)⁺=438 (method P).

### Example 145

### ((2R,4S,5S)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5537)

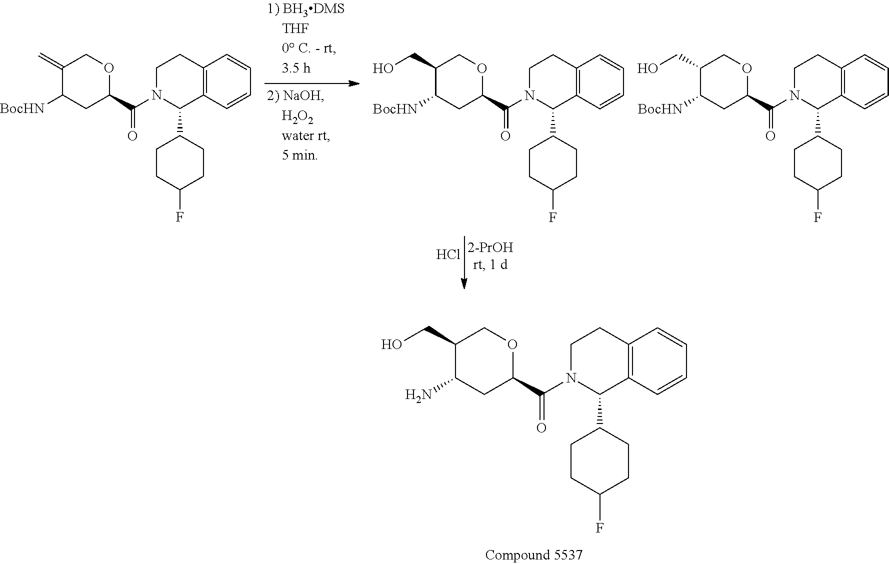

### Synthesis of tert-butyl ((2R,4S,5S)-2-((S)-1-(4-fluorophenyl)-1,2,3,4-tetrahydroisoquinoline-2-carbonyl)-5-(hydroxymethyl)tetrahydro-2H-pyran-4-yl)carbamate and tert-butyl ((2R,4S,5R)-2-((S)-1-(4-fluorophenyl)-1,2,3,4-tetrahydroisoquinoline-2-carbonyl)-5-(hydroxymethyl)tetrahydro-2H-pyran-4-yl)carbamate

At 0° C. under an N₂ atmosphere, borane dimethyl sulfide complex (2 M in tetrahydrofuran, 0.134 mL, 0.268 mmol) was added to a solution of tert-butyl ((2R,4S)-2-((S)-1-(4-fluorophenyl)-1,2,3,4-tetrahydroisoquinoline-2-carbonyl)-5-methylenetetrahydro-2H-pyran-4-yl)carbamate (see Compound 5518, 100 mg, 0.214 mmol) in tetrahydrofuran (dry, 2.5 mL). After 75 minutes, another portion of borane dimethyl sulfide complex (2 M in tetrahydrofuran, 0.134 mL, 0.268 mmol) was added and the reaction mixture was allowed to warm to room temperature. After 2 hours, sodium hydroxide (1 M, 1.286 mL, 1.286 mmol) was slowly added followed by hydrogen peroxide (30% aqueous, 0.219 mL, 2.143 mmol). After 5 minutes, the mixture was diluted with dichloromethane (15 mL). The organic layer was passed through a hydrophobic frit evaporated under reduced pressure. The residue was purified by flash column chromatography (silica, 0 to 50% ethyl acetate in heptane) and preparative SFC (method AT) to give tert-butyl ((2R,4S,5S)-2-((S)-1-(4-fluorophenyl)-1,2,3,4-tetrahydroisoquinoline-2-carbonyl)-5-(hydroxymethyl)tetrahydro-2H-pyran-4-yl)carbamate (12.6 mg) as the first eluting SFC isomer and tert-butyl ((2R,4S,5R)-2-((S)-1-(4-fluorophenyl)-1,2,3,4-tetrahydroisoquinoline-2-carbonyl)-5-(hydroxymethyl)tetrahydro-2H-pyran-4-yl)carbamate as the second eluting SFC isomer.

First eluting SFC isomer: SFC: RT=2.28 min., (M+H)⁺=485 (method AU).

Second eluting SFC isomer: SFC: RT=2.37 min., (M+H)⁺=485 (method AU).

### Synthesis of ((2R,4S,5S)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5537)

HCl (5-6 M in 2-propanol, 0.5 mL, 2.75 mmol) was added to a solution of tert-butyl ((2R,4S,5S)-2-((S)-1-(4-fluorophenyl)-1,2,3,4-tetrahydroisoquinoline-2-carbonyl)-5-(hydroxymethyl)tetrahydro-2H-pyran-4-yl)carbamate (12.6 mg, 0.026 mmol) in 2-propanol (1.0 mL). After 1 day, the mixture was partitioned between dichloromethane (15 mL) and saturated aqueous NaHCO₃ (5 mL). The organic layer was passed through a hydrophobic frit and evaporated under reduced pressure. The residue was dissolved in methanol, brought onto an SCX-2 column (1 g) and eluted with methanol (5 column volumes). Next, the column was eluted with ammonia in methanol (1 M). The basic fraction was concentrated to dryness under reduced pressure. The residue was lyophilized from a mixture of acetonitrile and water (1:1, 4 mL) to give ((2R,4S,5S)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5537).

LCMS: 99%, RT=1.04 min., (M+H)⁺=385 (method P). SFC: 94%, RT=3.15 min., (M+H)⁺=385 (method W).

### Example 146

### ((2R,4S,5R)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5538)

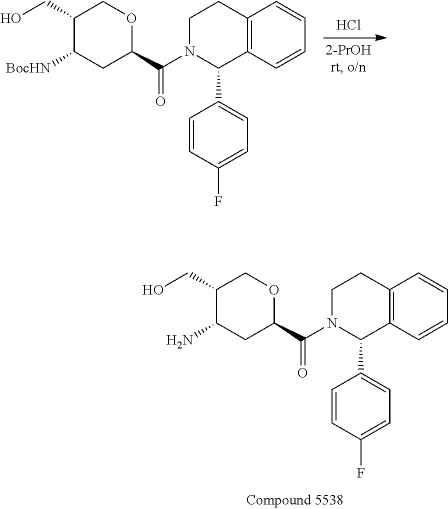 [SMILES: CC(C)(C)OC(=O)N[C@H]1C[C@H](C(=O)N2CCc3ccccc3[C@@H]2c2ccc(F)cc2)OC[C@H]1CO ; Cl ; N[C@H]1C[C@H](C(=O)N2CCc3ccccc3[C@@H]2c2ccc(F)cc2)OC[C@H]1CO]

### Synthesis of ((2R,4S,5R)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5538)

HCl (5-6 M in 2-propanol, 0.5 mL, 2.75 mmol) was added to a solution of tert-butyl ((2R,4S,5R)-2-((S)-1-(4-fluorophenyl)-1,2,3,4-tetrahydroisoquinoline-2-carbonyl)-5-(hydroxymethyl)tetrahydro-2H-pyran-4-yl)carbamate (see Compound 5537, 18.8 mg, 0.039 mmol) in 2-propanol (1.0 mL). After 1 day, the mixture was partitioned between dichloromethane (15 mL) and saturated aqueous NaHCO₃ (5 mL). The organic layer was passed through a hydrophobic frit and evaporated under reduced pressure. The residue was dissolved in methanol, brought onto an SCX-2 column (1 g) and eluted with methanol (5 column volumes). Next, the column was eluted with ammonia in methanol (1 M). The basic fraction was concentrated to dryness under reduced pressure. The residue was lyophilized from a mixture of acetonitrile and water (1:1, 4 mL) to give ((2R,4S,5R)-4-amino-5-(hydroxymethyl)tetrahydro-2H-pyran-2-yl)((S)-1-(4-fluorophenyl)-3,4-dihydroisoquinolin-2(1H)-yl)methanone (COMPOUND 5538).

LCMS: 91%, RT=2.59 min., (M+H)⁺=385 (method AK). SFC: 93%, RT=3.63 min., (M+H)⁺=385 (method W).

### Example 147

### ((2R,4R)-4-(aminomethyl)-4-hydroxytetrahydro-2H-pyran-2-yl)((S)-1-(4-fluorophenyl)-3,4-dihydroisoquinolin-2(1H)-yl)methanone (COMPOUND 5540)

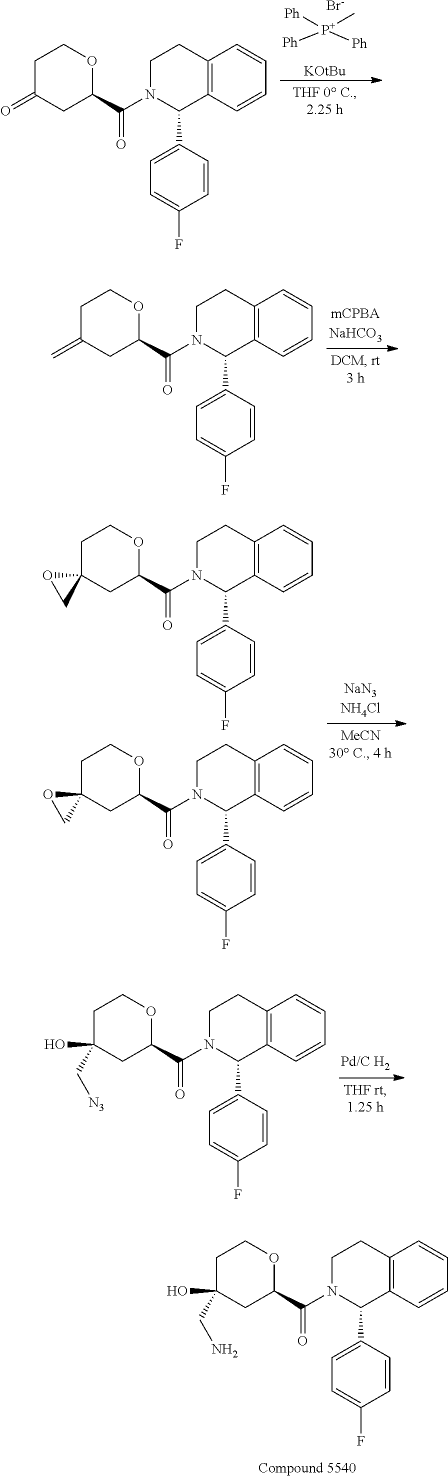

### Synthesis of ((S)-1-(4-fluorophenyl)-3,4-dihydroisoquinolin-2(1H)-yl)((R)-4-methylenetetrahydro-2H-pyran-2-yl)methanone

At 0° C., potassium tert-butoxide (1 M solution in tetrahydrofuran, 0.445 mL, 0.445 mmol) was added dropwise in 2 minutes to a suspension of methyltriphenylphosphonium bromide (167.6 mg, 0.469 mmol) in tetrahydrofuran (dry, 1.5 mL). After stirring for 30 minutes, a solution of (R)-2-((S)-1-(4-fluorophenyl)-1,2,3,4-tetrahydroisoquinoline-2-carbonyl)tetrahydro-4H-pyran-4-one (see Compound 5080, 149.8 mg, 0.424 mmol) in tetrahydrofuran (dry, 1.5 mL) was added dropwise in 2 minutes. After 1.75 hour, the reaction mixture was partitioned between a mixture of brine (10 mL), water (10 mL), and dichloromethane (15 mL). The organic layer was passed through a hydrophobic frit and evaporated under reduced pressure. The residue was purified by flash column chromatography (silica: 5 to 50% ethyl acetate in heptane) to give ((S)-1-(4-fluorophenyl)-3,4-dihydroisoquinolin-2(1H)-yl)((R)-4-methylenetetrahydro-2H-pyran-2-yl)methanone.

SFC: 99%, RT=3.18 min., (M+H)⁺=352 (method F).

### Synthesis of ((S)-1-(4-fluorophenyl)-3,4-dihydroisoquinolin-2(1H)-yl)((3R,5R)-1,6-dioxaspiro[2.5]octan-5-yl)methanone and ((S)-1-(4-fluorophenyl)-3,4-dihydroisoquinolin-2(1H)-yl)((3S,5R)-1,6-dioxaspiro[2.5]octan-5-yl)methanone

Sodium bicarbonate (64.5 mg, 0.768 mmol) and meta-chloroperbenzoic acid (70 wt %, 149.2 mg, 0.605 mmol) were added to a solution of ((S)-1-(4-fluorophenyl)-3,4-dihydroisoquinolin-2(1H)-yl)((R)-4-methylenetetrahydro-2H-pyran-2-yl)methanone (177 mg, 0.504 mmol) in dichloromethane (5 mL). After stirring for 3 hours, the mixture was partitioned between dichloromethane (20 mL) and saturated aqueous NaHCO₃ (10 mL). The organic layer was passed through a hydrophobic frit and evaporated under reduced pressure. The residue was purified by flash column chromatography (silica: 5 to 60% ethyl acetate in heptane) to give ((S)-1-(4-fluorophenyl)-3,4-dihydroisoquinolin-2(1H)-yl)((3R,5R)-1,6-dioxaspiro[2.5]octan-5-yl)methanone as the first eluting isomer and ((S)-1-(4-fluorophenyl)-3,4-dihydroisoquinolin-2(1H)-yl)((3S,5R)-1,6-dioxaspiro[2.5]octan-5-yl)methanone as the second eluting isomer.

First eluting isomer: SFC: 98%, RT=2.03 min., (M+H)⁺=368 (method V).

Second eluting isomer: SFC: 96%, RT=2.52 min., (M+H)⁺=368 (method V).

### Synthesis of ((2R)-4-(azidomethyl)-4-hydroxytetrahydro-2H-pyran-2-yl)((S)-1-(4-fluorophenyl)-3,4-dihydroisoquinolin-2(1H)-yl)methanone

Acetic Acid (61 μL) was added to a solution of sodium azide (21.43 mg, 0.330 mmol) in water (106 μL) and this was added to a screwcap vial containing ((S)-1-(4-fluorophenyl)-3,4-dihydroisoquinolin-2(1H)-yl)((3R,5R)-1,6-dioxaspiro[2.5]octan-5-yl)methanone (28.5 mg, 0.066 mmol). The vial was capped and heated to 30° C. After 4 hours, the reaction mixture was partitioned between a mixture of brine and water (2:1, 5 mL) and dichloromethane (10 mL). The organic layer was separated, washed with saturated aqueous NaHCO₃ (5 mL), passed through a hydrophobic frit, and evaporated under reduced pressure. The residue was purified by flash column chromatography (silica, 20 to 70% ethyl acetate in heptane) to give ((2R,4R)-4-(azidomethyl)-4-hydroxytetrahydro-2H-pyran-2-yl)((S)-1-(4-fluorophenyl)-3,4-dihydroisoquinolin-2(1H)-yl)methanone.

LCMS: 97%, RT=1.96 min., (M+H)⁺=411 (method A).

### Synthesis of ((2R,4R)-4-(aminomethyl)-4-hydroxytetrahydro-2H-pyran-2-yl)((S)-1-(4-fluorophenyl)-3,4-dihydroisoquinolin-2(1H)-yl)methanone (COMPOUND 5540)

A solution of ((2R,4R)-4-(azidomethyl)-4-hydroxytetrahydro-2H-pyran-2-yl)((S)-1-(4-fluorophenyl)-3,4-dihydroisoquinolin-2(1H)-yl)methanone in tetrahydrofuran (2 mL) was hydrogenated in the presence of palladium (10 wt % on carbon, 7.5 mg, 3.53 μmol) at atmospheric hydrogen pressure. After stirring for 1.25 hour, the mixture was diluted with dichloromethane and filtered. The filtrate was concentrated under reduced pressure. The residue was dissolved in methanol, brought onto an SCX-2 column (1 g), and eluted with methanol (5 column volumes). Next, the column was eluted with ammonia in methanol (1 M). The basic fraction was concentrated to dryness under reduced pressure and the residue was lyophilised from a mixture of acetonitrile and water (1:1, 4 mL) to give ((2R,4R)-4-(aminomethyl)-4-hydroxytetrahydro-2H-pyran-2-yl)((S)-1-(4-fluorophenyl)-3,4-dihydroisoquinolin-2(1H)-yl)methanone (COMPOUND 5540).

LCMS: 94%, RT=1.01 min., (M+H)⁺=382 (method P).

### Example 148

### ((2R,4S,5R)-4-amino-5-((2,2,2-trifluoroethyl)amino)tetrahydro-2H-pyran-2-yl)((S)-1-(4-fluorophenyl)-3,4-dihydroisoquinolin-2(1H)-yl)methanone (COMPOUND 5539)

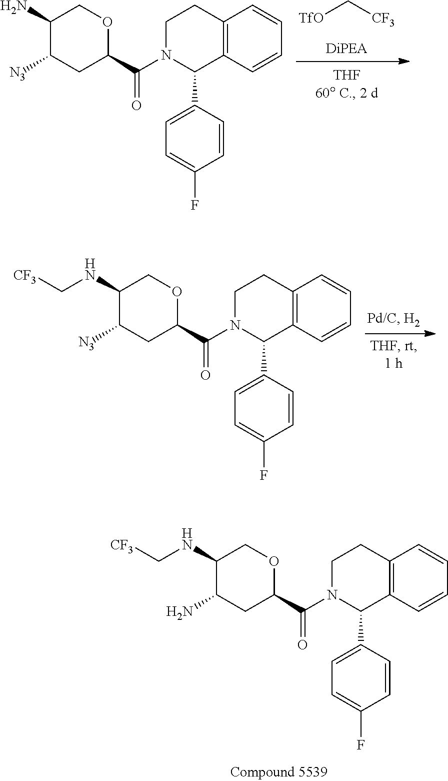

### Synthesis of ((2R,4S,5R)-4-azido-5-((2,2,2-trifluoroethyl)amino)tetrahydro-2H-pyran-2-yl)((S)-1-(4-fluorophenyl)-3,4-dihydroisoquinolin-2(1H)-yl)methanone

2,2,2-Trifluoroethyl trifluoromethanesulfonate (0.067 mL, 0.465 mmol) was added to a solution of ((2R,4S,5R)-5-amino-4-azidotetrahydro-2H-pyran-2-yl)((S)-1-(4-fluorophenyl)-3,4-dihydroisoquinolin-2(1H)-yl)methanone (see Compound 5512, 92 mg, 0.233 mmol) and N,N-diisopropylethylamine (0.122 mL, 0.698 mmol) in tetrahydrofuran (2.5 mL) and the mixture was warmed to 60° C. After stirring overnight, additional portions of 2,2,2-trifluoroethyl trifluoromethanesulfonate (0.034 mL, 0.233 mmol) and N,N-diisopropylethylamine (0.061 mL, 0.349 mmol) were added and stirring was continued overnight. Then the mixture was concentrated under reduced pressure and the residue was purified by flash column chromatography (silica, 5 to 40% ethyl acetate in heptane) to give ((2R,4S,5R)-4-azido-5-((2,2,2-trifluoroethyl)amino)tetrahydro-2H-pyran-2-yl)((S)-1-(4-fluorophenyl)-3,4-dihydroisoquinolin-2(1H)-yl)methanone.

LCMS: 100%, RT=2.21 min., (M+H)⁺=478 (method A).

### Synthesis of ((2R,4S,5R)-4-amino-5-((2,2,2-trifluoroethyl)amino)tetrahydro-2H-pyran-2-yl)((S)-1-(4-fluorophenyl)-3,4-dihydroisoquinolin-2(1H)-yl)methanone (COMPOUND 5539)

A solution of ((2R,4S,5R)-4-azido-5-((2,2,2-trifluoroethyl)amino)tetrahydro-2H-pyran-2-yl)((S)-1-(4-fluorophenyl)-3,4-dihydroisoquinolin-2(1H)-yl)methanone (95 mg, 0.199 mmol) in tetrahydrofuran (4 mL) was hydrogenated in the presence of palladium (10 wt % on carbon, containing 50% water, 60 mg, 0.028 mmol) at atmospheric hydrogen pressure. After stirring for 1 hour, the mixture was diluted with ethyl acetate and filtered over a 0.45 μm nylon filter. The filtrate was evaporated under reduced pressure. The residue was purified by acidic preparative MPLC (Linear Gradient: t=0 min 5% A; t=1 min 5% A; t=2 min 20% A; t=17 min 60% A; t=18 min 100%; t=23 min 100% A; detection: 220 nm). The product fractions were combined and basified with saturated aqueous NaHCO₃ and then partially concentrated under reduced pressure to remove the acetonitrile. The aqueous residue was extracted with dichloromethane (twice). The combined organic layers were dried over Na₂SO₄ and concentrated under reduced pressure. The residue was lyophilized from a mixture of acetonitrile and water (1:1, 4 mL) to give ((2R,4S,5R)-4-amino-5-((2,2,2-trifluoroethyl)amino)tetrahydro-2H-pyran-2-yl)((S)-1-(4-fluorophenyl)-3,4-dihydroisoquinolin-2(1H)-yl)methanone (COMPOUND 5539).

LCMS: 100%, RT=1.17 min., (M+H)⁺=452 (method P).

### Example 149

### 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)ethan-1-one (COMPOUND 5543)

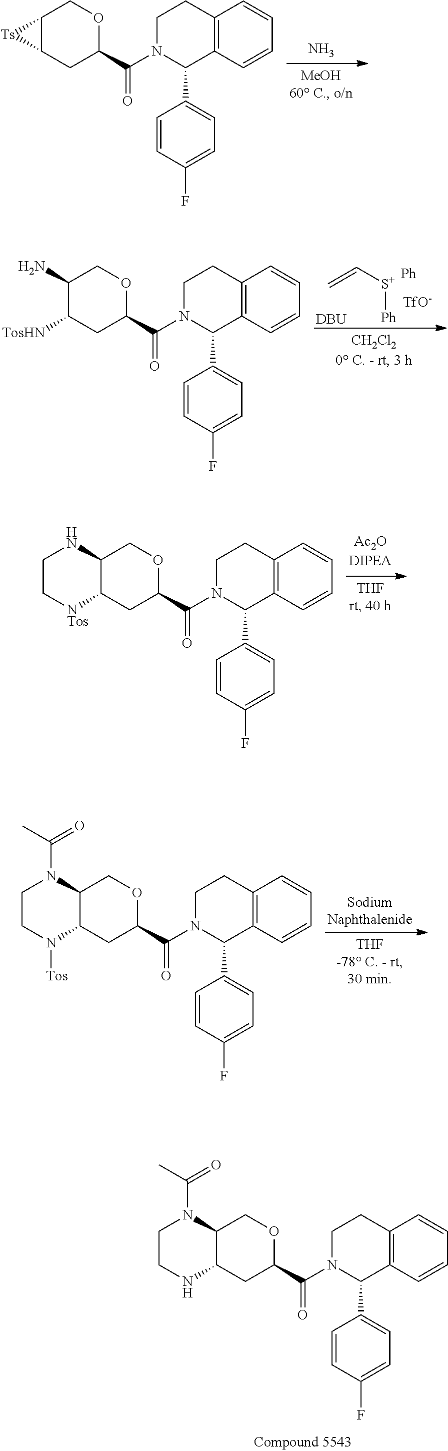

### Synthesis of N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide

A suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 1.50 g, 2.96 mmol) in ammonia in methanol (7 M, 15 mL, 105 mmol) was heated in a capped microwave vial at 60° C., overnight. Then, the reaction mixture was concentrated to dryness under reduced pressure to give N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide (1.584 g) as a white solid that was used as such.

### Synthesis of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone

At 0° C. under nitrogen atmosphere, 1,8-diazabicyclo[5.4.0]undec-7-ene (0.905 mL, 6.05 mmol), followed by dropwise addition of ethenyldiphenylsulfanium trifluoromethanesulfonate (1.370 mL, 3.78 mmol), were added to a suspension of N-((2R,4S,5R)-5-amino-2-((S)-1-(4-fluorophenyl)-1,2,3,4-tetrahydroisoquinoline-2-carbonyl)tetrahydro-2H-pyran-4-yl)-4-methylbenzenesulfonamide (1.584 g, 3.03 mmol) in dichloromethane (dry, 50 mL). After 10 minutes, the mixture was allowed to slowly warm to room temperature and stirring was continued for 3 hours. Then, the reaction mixture was washed with aqueous HCl (1 M, 20 mL) and saturated aqueous NaHCO₃ (20 mL), dried over Na₂SO₄, and evaporated under reduced pressure. The residue was purified by flash column chromatography (silica, 5 to 100% ethyl acetate in heptane) to give ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone.

LCMS: 97%, RT=1.79 min., (M+H)⁺=550 (method A).

### Synthesis of 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)ethan-1-one

Acetic anhydride (0.032 mL, 0.341 mmol) was added to a solution of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone (138 mg, 0.251 mmol) and N,N-diisopropylethylamine (0.143 mL, 0.819 mmol) in tetrahydrofuran (dry, 2.5 mL). After 40 hours, the reaction mixture was partitioned between dichloromethane (7.5 mL) and saturated aqueous NaHCO₃ (7.5 mL). The layers were separated and the aqueous phase was extracted with dichloromethane (7.5 mL). The combined organic layers were dried over Na₂SO₄ and evaporated under reduced pressure. The residue was purified by flash column chromatography on silica (0 to 100% ethyl acetate in heptane) to give 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)ethan-1-one.

LCMS: 99%, RT=2.26 min., (M+H)⁺=592 (method A).

### Synthesis of 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)ethan-1-one (COMPOUND 5543)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.735 mL, 0.367 mmol) was added to a solution of 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)ethan-1-one (118 mg, 0.199 mmol) in tetrahydrofuran (dry, 2.5 mL). After 15 minutes, an additional portion of sodium naphthalenide (0.5M in tetrahydrofuran, 0.423 mL, 0.211 mmol) was added. After 15 minutes, the reaction mixture was quenched by addition of methanol (0.4 mL) and stored in the freezer overnight. Then, the mixture was concentrated to dryness under reduced pressure. The residue was purified by flash column chromatography (silica, 0 to 7.5% (7M NH₃ in methanol) in dichloromethane) and preparative SFC (method BA) to give 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)ethan-1-one (COMPOUND 5543) as the first eluting isomer on SFC, after lyophilization from a mixture of acetonitrile and water (1:1, 4 mL).

LCMS: 97%, RT=1.05 min., (M+H)⁺=438 (method P). SFC: 100%, RT=4.66 min., (M+H)⁺=438 (method AD).

### Example 150

### ((S)-1-(4-fluorophenyl)-3,4-dihydroisoquinolin-2(1H)-yl)((4aR,7R,8aS)-4-(2,2,2-trifluoroethyl)octahydro-2H-pyrano[3,4-b]pyrazin-7-yl)methanone (Compound 5545)

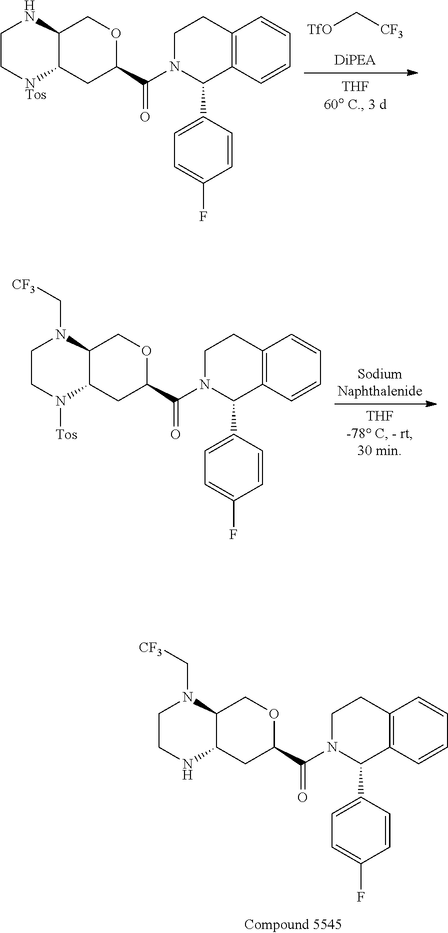

### Synthesis of ((S)-1-(4-fluorophenyl)-3,4-dihydroisoquinolin-2(1H)-yl)((4aR,7R,8aS)-1-tosyl-4-(2,2,2-trifluoroethyl)octahydro-2H-pyrano[3,4-b]pyrazin-7-yl)methanone

N,N-diisopropylethylamine (0.143 mL, 0.819 mmol) and 2,2,2-trifluoroethyl trifluoromethanesulfonate (0.059 mL, 0.409 mmol) were added to a solution of ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone (see Compound 5462, 150 mg, 0.273 mmol) in tetrahydrofuran (dry, 2.5 mL) and the mixture was heated to 60° C. After 4 hours, extra quantities of N,N-diisopropylethylamine (0.143 mL, 0.819 mmol) and 2,2,2-trifluoroethyl trifluoromethanesulfonate (0.059 mL, 0.409 mmol) were added and stirring was continued for 2 days. Then, an extra quantity of 2,2,2-trifluoroethyl trifluoromethanesulfonate (0.059 mL, 0.409 mmol) was added. After 1 more day, the reaction mixture was cooled to room temperature and concentrated under reduced pressure. The residue was purified by flash column chromatography (silica, 0 to 50% ethyl acetate in heptane) to give ((S)-1-(4-fluorophenyl)-3,4-dihydroisoquinolin-2(1H)-yl)((4aR,7R,8aS)-1-tosyl-4-(2,2,2-trifluoroethyl)octahydro-2H-pyrano[3,4-b]pyrazin-7-yl)methanone.

LCMS: 99%, RT=2.31 min., (M+H)⁺=632 (method A).

### Synthesis of ((S)-1-(4-fluorophenyl)-3,4-dihydroisoquinolin-2(1H)-yl)((4aR,7R,8aS)-4-(2,2,2-trifluoroethyl)octahydro-2H-pyrano[3,4-b]pyrazin-7-yl)methanone (Compound 5545)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 0.792 mL, 0.396 mmol) was added to a solution of ((S)-1-(4-fluorophenyl)-3,4-dihydroisoquinolin-2(1H)-yl)((4aR,7R,8aS)-1-tosyl-4-(2,2,2-trifluoroethyl)octahydro-2H-pyrano[3,4-b]pyrazin-7-yl)methanone (119 mg, 0.188 mmol) in tetrahydrofuran (dry, 2.5 mL). After 15 minutes, an additional portion of sodium naphthalenide (0.5M in tetrahydrofuran, 0.396 mL, 0.198 mmol) was added. After 15 minutes, the reaction mixture was quenched by addition of saturated aqueous ammonium chloride (2.0 mL), diluted with dichloromethane (5 mL) and stored in the freezer for 7 days. Then, the layers were separated and the aqueous phase was extracted with dichloromethane (5 mL). The combined organic phase was passed through a hydrophobic frit and evaporated under reduced pressure. The residue was purified by flash column chromatography (silica, 0 to 7.5% (7M NH₃ in methanol) in dichloromethane) and preparative SFC (method AN) to give ((S)-1-(4-fluorophenyl)-3,4-dihydroisoquinolin-2(1H)-yl)((4aR,7R,8aS)-4-(2,2,2-trifluoroethyl)octahydro-2H-pyrano[3,4-b]pyrazin-7-yl)methanone (Compound 5545) as the first eluting isomer on SFC, as after lyophilization from a mixture of acetonitrile and water (1:1, 4 mL).

LCMS: 98%, RT=1.22 min., (M+H)⁺=478 (method P). SFC: 99%, RT=2.70 min., (M+H)⁺=478 (method AD).

### Example 151

### 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)propan-1-one (Compound 5544)

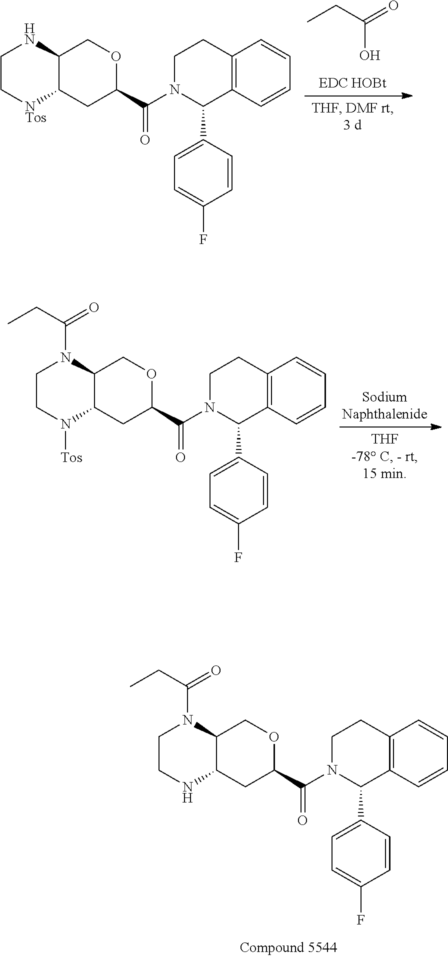

### Synthesis of 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)propan-1-one

N-(3-dimethylaminopropyl)-N′-ethylcarbodiimide hydrochloride (65.4 mg, 0.341 mmol) and 1-hydroxybenzotriazole hydrate (4.18 mg, 0.027 mmol) were added to a solution of propionic acid (0.026 mL, 0.341 mmol) and ((S)-1-(4-fluorophenyl)-3,4-dihydroisoquinolin-2(1H)-yl)((4aR,7R,8aS)-1-tosyloctahydro-2H-pyrano[3,4-b]pyrazin-7-yl)methanone (see Compound 5462, 150 mg, 0.273 mmol) in a mixture of tetrahydrofuran (dry, 2.5 mL) and N,N-dimethylformamide (dry, 0.5 mL). After 2 days, extra quantities of propionic acid (10.2 μL, 0.136 mmol), followed by N-(3-dimethylaminopropyl)-N′-ethylcarbodiimide hydrochloride (26.2 mg, 0.136 mmol) and 1-hydroxybenzotriazole hydrate (4.18 mg, 0.027 mmol) were added and stirring was continued for 1 day. Then, the reaction mixture was partitioned between ethyl acetate (25 mL) and water (25 mL). The organic layer was washed with half saturated brine (3×10 mL) and brine (10 mL), dried over Na₂SO₄, and evaporated under reduced pressure. The residue was purified by flash column chromatography (silica, 5 to 100% ethyl acetate in heptane) to give 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)propan-1-one.

LCMS: 99%, RT=2.17 min., (M+H)⁺=606 (method A).

### Synthesis of 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)propan-1-one (Compound 5544)

Under argon atmosphere, lumps of sodium (90 mg, 3.90 mmol) were added to a solution of naphthalene (500 mg, 3.90 mmol) in tetrahydrofuran (dry, 8 mL). The mixture was stirred for 2 hours till all sodium was dissolved to give a solution of sodium naphthalenide (0.5 M in tetrahydrofuran). At −78° C. under argon atmosphere, sodium naphthalenide (0.5M in tetrahydrofuran, 1.783 mL, 0.891 mmol) was added to a solution of 1-((4aR,7R,8aS)-7-((S)-1-(4-fluorophenyl)-1,2,3,4-tetrahydroisoquinoline-2-carbonyl)-1-tosyloctahydro-4H-pyrano[3,4-b]pyrazin-4-yl)propan-1-one (125 mg, 0.206 mmol) in tetrahydrofuran (dry, 2.5 mL). After 15 minutes, the reaction mixture was quenched by addition of methanol (0.4 mL) and stored in the freezer over the weekend. Then, the mixture was concentrated to dryness under reduced pressure. The residue was purified by flash column chromatography (silica, 0 to 6% (7M NH₃ in methanol) in dichloromethane) and preparative SFC (method BA) to give 1-((4aR,7R,8aS)-7-((S)-1-(4-fluorophenyl)-1,2,3,4-tetrahydroisoquinoline-2-carbonyl)octahydro-4H-pyrano[3,4-b]pyrazin-4-yl)propan-1-one (Compound 5544) as the first eluting isomer on SFC, after lyophilization from a mixture of acetonitrile and water (1:1, 4 mL).

LCMS: 97%, RT=1.11 min., (M+H)⁺=452 (method P). SFC: 99%, RT=4.55 min., (M+H)⁺=452 (method AD).

### Example 152

### ((S)-1-(4-fluorophenyl)-3,4-dihydroisoquinolin-2(1H)-yl)((3S,4aR,7R,8aS)-3-methyloctahydropyrano[3,4-b][1,4]oxazin-7-yl)methanone (Compound 5471)

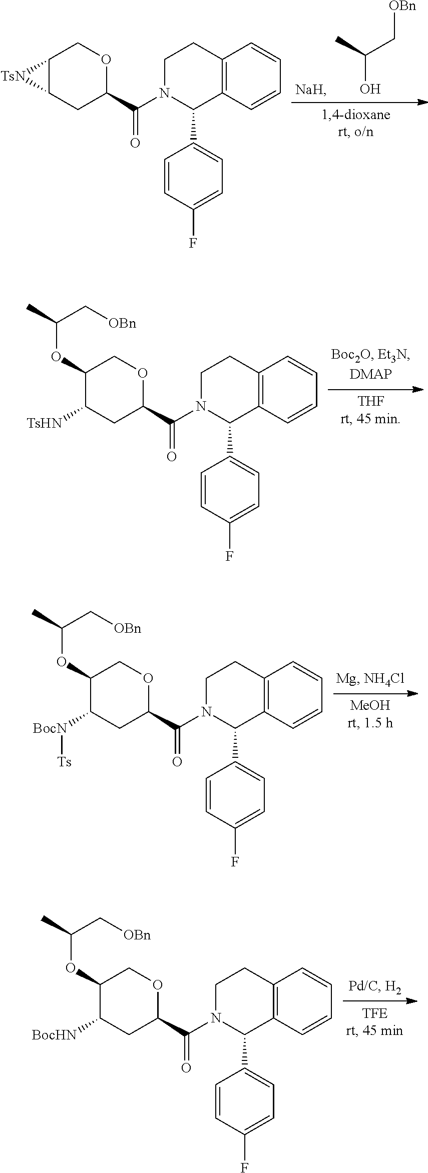

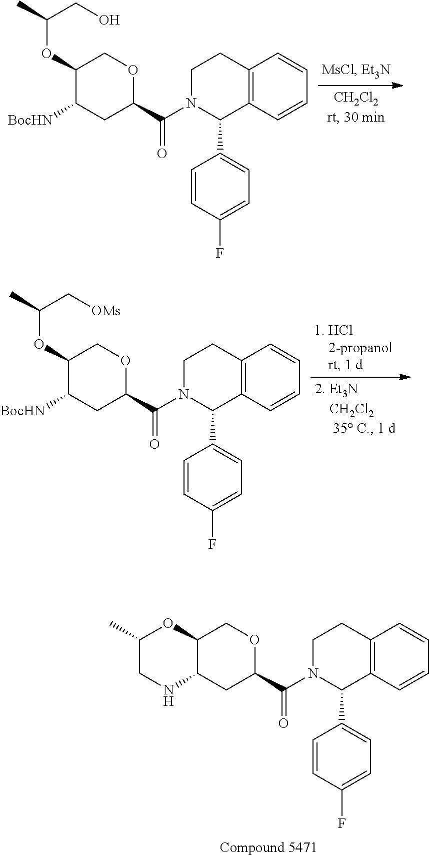 [SMILES: C[C@@H](CO)O[C@H]1CO[C@@H](C(=O)N2CCc3ccccc3[C@@H]2c2ccc(F)cc2)C[C@@H]1NC(=O)OC(C)(C)C ; C[C@@H](COS(C)(=O)=O)O[C@H]1CO[C@@H](C(=O)N2CCc3ccccc3[C@@H]2c2ccc(F)cc2)C[C@@H]1NC(=O)OC(C)(C)C ; C[C@H]1CN[C@H]2C[C@H](C(=O)N3CCc4ccccc4[C@@H]3c3ccc(F)cc3)OC[C@@H]2O1]

### Synthesis of N-((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide

Under an argon atmosphere, sodium hydride (60% dispersion in mineral oil, 35.5 mg, 0.888 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 300 mg, 0.592 mmol) and (S)-(−)-1-benzyloxy-2-propanol (492 mg, 2.96 mmol) in 1,4-dioxane (5 mL). After stirring overnight, the mixture was diluted with saturated aqueous NH₄Cl and extracted with ethyl acetate (2×). The combined organics were washed with brine, dried over Na₂SO₄, and evaporated under reduced pressure. The residue was purified by flash column chromatography (silica, 10 to 65% ethyl acetate in heptane) to give N-((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 90%, RT=2.25 min., (M+H)⁺=673 (method A).

### Synthesis of tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.073 mL, 0.526 mmol), di-tert-butyl dicarbonate (0.115 mg, 0.526 mmol), and 4-dimethylaminopyridine (catalytic amount) were added to a solution of N-((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide (177 mg, 0.263 mmol) in tetrahydrofuran (dry, 5 mL). After 45 minutes, the mixture was concentrated under reduced pressure. The residue was purified by flash column chromatography (silica, 5 to 40% ethyl acetate in heptane) to give tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate.

LCMS: 95%, RT=2.44 min., (M+H)⁺=773 (method A).

### Synthesis of tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate

Ammonium chloride (118 mg, 2.20 mmol) and magnesium (267 mg, 11.00 mmol) were added to a solution of tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (170 mg, 0.220 mmol) in methanol (5 mL). After 1.5 hours, the mixture was diluted with saturated aqueous NH₄Cl (15 mL) and stirred for 10 minutes. Then, the mixture was extracted with dichloromethane (2×15 mL). The combined organics were dried over Na₂SO₄ and evaporated under reduced pressure. The residue was purified by flash column chromatography (silica, 5 to 50% ethyl acetate in heptane) to give tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate.

LCMS: 95%, RT=2.27 min., (M+Na)⁺=619 (method A).

### Synthesis of tert-butyl ((2R,4S,5R)-2-((S)-1-(4-fluorophenyl)-1,2,3,4-tetrahydroisoquinoline-2-carbonyl)-5-(((S)-1-hydroxypropan-2-yl)oxy)tetrahydro-2H-pyran-4-yl)carbamate

A solution of tert-butyl ((2R,4S,5R)-5-(((S)-1-(benzyloxy)propan-2-yl)oxy)-2-((S)-1-(4-fluorophenyl)-1,2,3,4-tetrahydroisoquinoline-2-carbonyl)tetrahydro-2H-pyran-4-yl)carbamate (102 mg, 0.165 mmol) in 2,2,2-trifluoroethanol (3 mL) was hydrogenated in the presence of palladium (10 wt % on carbon, containing 50% water, 50 mg, 0.023 mmol) at atmospheric hydrogen pressure. After stirring for 45 minutes, the mixture was filtered over a 0.45 μm nylon filter. The filtrate was concentrated under reduced pressure to give tert-butyl ((2R,4S,5R)-2-((S)-1-(4-fluorophenyl)-1,2,3,4-tetrahydroisoquinoline-2-carbonyl)-5-(((S)-1-hydroxypropan-2-yl)oxy)tetrahydro-2H-pyran-4-yl)carbamate.

LCMS: 93%, RT=2.02 min., (M+H)⁺=529 (method A).

### Synthesis of (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propy methanesulfonate

Triethylamine (0.051 mL, 0.369 mmol) and methanesulfonyl chloride (0.019 mL, 0.246 mmol) were added to a solution of tert-butyl ((2R,4S,5R)-2-((S)-1-(4-fluorophenyl)-1,2,3,4-tetrahydroisoquinoline-2-carbonyl)-5-(((R)-1-hydroxypropan-2-yl)oxy)tetrahydro-2H-pyran-4-yl)carbamate (65 mg, 0.123 mmol) in dichloromethane (2 mL). After 30 minutes, the mixture was diluted with saturated aqueous NaHCO₃ and stirred for 15 minutes. The layers were separated over a phase separation filter and the organic filtrate was evaporated under reduced pressure to yield (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propyl methanesulfonate.

LCMS: 95%, RT=2.10 min., (M+H)⁺=607 (method A).

### Synthesis of ((S)-1-(4-fluorophenyl)-3,4-dihydroisoquinolin-2(1H)-yl)((3S,4aR,7R,8aS)-3-methyloctahydropyrano[3,4-b][1,4]oxazin-7-yl)methanone (Compound 5471)

HCl (5-6 M in 2-propanol, 2 mL, 11.0 mmol) was added to a solution of (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)propyl methanesulfonate (65 mg, 0.107 mmol) in 2-propanol (7 mL). After stirring for 1 day, the mixture was diluted with saturated aqueous NaHCO₃ (30 mL) and dichloromethane (25 mL) and stirred vigorously for 15 minutes. The layers were separated and the aqueous phase was extracted with dichloromethane (25 mL). The combined organics were evaporated under reduced pressure. The residue was dissolved in dichloromethane and triethylamine (0.045 mL, 0.321 mmol) was added. After stirring overnight, the mixture was warmed to 35° C. and stirred for 1 day. Then, the mixture was concentrated under reduced pressure. The residue was dissolved in methanol, brought onto an SCX-2 column (1 g), and eluted with methanol (5 column volumes). Next, the column was eluted with ammonia in methanol (2 M). The basic fraction was concentrated to dryness under reduced pressure. The residue was purified by preparative SFC (method S) to give ((S)-1-(4-fluorophenyl)-3,4-dihydroisoquinolin-2(1H)-yl)((3S,4aR,7R,8aS)-3-methyloctahydropyrano[3,4-b][1,4]oxazin-7-yl)methanone (Compound 5471) after lyophilization from a mixture of acetonitrile and water (1:1, 4 mL).

LCMS: 96%, RT=1.12 min., (M+H)⁺=411 (method P). SFC: 95%, RT=3.39 min., (M+H)⁺=411 (method G).

### Example 153

### ((S)-1-(4-fluorophenyl)-3,4-dihydroisoquinolin-2(1H)-yl)((3R,4aR,7R,8aS)-3-(trifluoromethyl)octahydropyrano[3,4-b][1,4]oxazin-7-yl)methanone (Compound 5474)

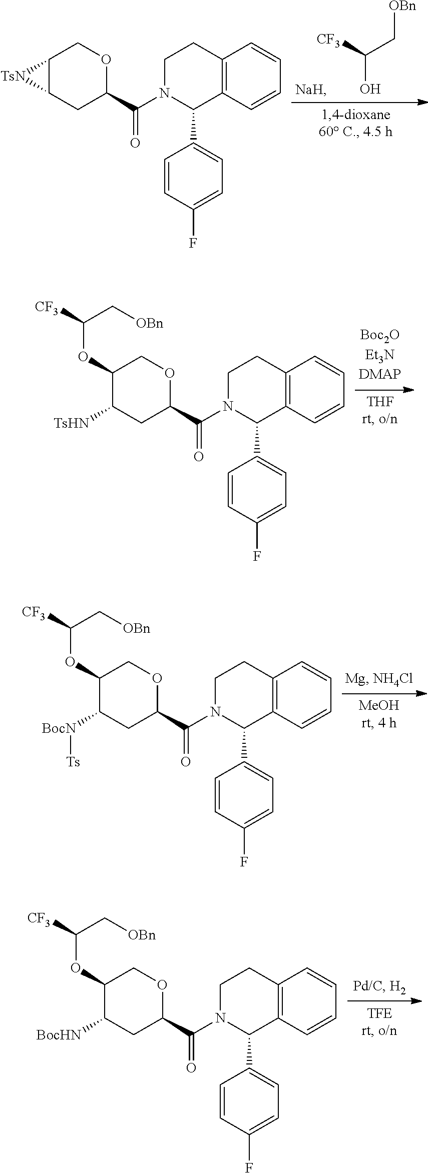

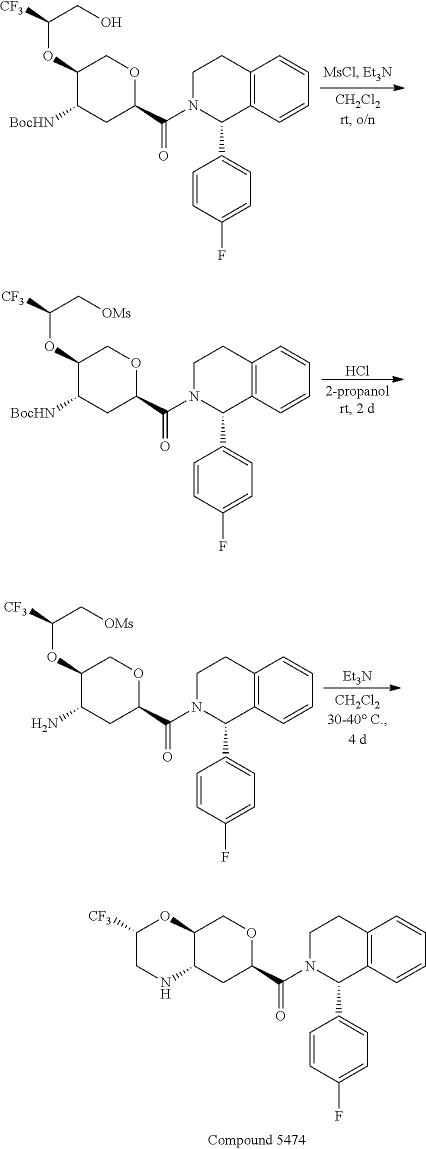 [SMILES: CC(C)(C)OC(=O)N[C@H]1C[C@H](C(=O)N2CCc3ccccc3[C@@H]2c2ccc(F)cc2)OC[C@@H]1O[C@H](CO)C(F)(F)F ; "Et3N" ; CC(C)(C)OC(=O)N[C@H]1C[C@H](C(=O)N2CCc3ccccc3[C@@H]2c2ccc(F)cc2)OC[C@@H]1O[C@H](COS(C)(=O)=O)C(F)(F)F ; Cl ; CS(=O)(=O)OC[C@@H](O[C@H]1CO[C@@H](C(=O)N2CCc3ccccc3[C@@H]2c2ccc(F)cc2)C[C@@H]1N)C(F)(F)F ; O=C([C@H]1C[C@@H]2NC[C@H](C(F)(F)F)O[C@H]2CO1)N1CCc2ccccc2[C@@H]1c1ccc(F)cc1]

### Synthesis of N-((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide

Under an argon atmosphere, sodium hydride (60% dispersion in mineral oil, 59.2 mg, 1.480 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 500 mg, 0.987 mmol) and (R)-3-(benzyloxy)-1,1,1-trifluoropropan-2-ol (652 mg, 2.96 mmol) in 1,4-dioxane (20 mL) and the mixture was heated to 60° C. After stirring for 4.5 hours, the mixture was diluted with saturated aqueous NH₄Cl (50 mL) and water (few mL) and extracted with ethyl acetate (2×50 mL). The combined organics were dried over Na₂SO₄ and evaporated under reduced pressure. The residue was purified by flash column chromatography (silica, 5 to 40% ethyl acetate in heptane) to give N-((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 93%, RT=2.33 min., (M+H)⁺=727 (method A).

### Synthesis of tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.182 mL, 1.307 mmol), di-tert-butyl dicarbonate (0.285 mg, 1.307 mmol)), and 4-dimethylaminopyridine (7.98 mg, 0.065 mmol) were added to a solution of N-((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide (475 mg, 0.654 mmol) in tetrahydrofuran (dry, 8 mL). After stirring overnight, the mixture was concentrated under reduced pressure. The residue was purified by flash column chromatography (silica, 0 to 50% ethyl acetate in heptane) to give tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate.

LCMS: 97%, RT=2.48 min., (M+H)⁺=827 (method A).

### Synthesis of tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate

Ammonium chloride (312 mg, 5.84 mmol) and magnesium (709 mg, 29.2 mmol) were added to a solution of tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (482.5 mg, 0.583 mmol) in methanol (17 mL). After stirring overnight, an extra quantity of magnesium (126 mg, 5.20 mmol) was added and stirring was continued for 4 hours. After this, the mixture was stored in the freezer over the weekend. Then, the mixture was diluted with saturated aqueous NH₄Cl (40 mL), stirred for 10 minutes, and extracted with dichloromethane (2×40 mL). The combined organics were dried over Na₂SO₄ and evaporated under reduced pressure to give tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate that was used as such.

LCMS: 90%, RT=2.35 min., (M+H)⁺=673 (method A).

### Synthesis of tert-butyl ((2R,4S,5R)-2-((S)-1-(4-fluorophenyl)-1,2,3,4-tetrahydroisoquinoline-2-carbonyl)-5-(((R)-1,1,1-trifluoro-3-hydroxypropan-2-yl)oxy)tetrahydro-2H-pyran-4-yl)carbamate

A solution of tert-butyl ((2R,4S,5R)-5-(((R)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate (156 mg, 0.232 mmol) in 2,2,2-trifluoroethanol (3.5 mL) was hydrogenated in the presence of palladium (10 wt % on carbon, containing 50% water, 61.7 mg, 0.029 mmol) at atmospheric hydrogen pressure. After stirring overnight, the mixture was filtered over a 0.45 μm nylon filter. The filtrate was concentrated under reduced pressure to give tert-butyl ((2R,4S,5R)-2-((S)-1-(4-fluorophenyl)-1,2,3,4-tetrahydroisoquinoline-2-carbonyl)-5-(((R)-1,1,1-trifluoro-3-hydroxypropan-2-yl)oxy)tetrahydro-2H-pyran-4-yl)carbamate (131 mg) as a colorless oil.

LCMS: 84%, RT=2.14 min., (M+H)⁺=583 (method A).

### Synthesis of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate

Triethylamine (0.094 mL, 0.674 mmol) and methanesulfonyl chloride (0.035 mL, 0.449 mmol) were added to a solution of tert-butyl ((2R,4S,5R)-2-((S)-1-(4-fluorophenyl)-1,2,3,4-tetrahydroisoquinoline-2-carbonyl)-5-(((R)-1,1,1-trifluoro-3-hydroxypropan-2-yl)oxy)tetrahydro-2H-pyran-4-yl)carbamate (131 mg, 0.225 mmol) in dichloromethane (2.5 mL). After stirring overnight, the reaction mixture was purified by flash column chromatography (silica, 0 to 60% ethyl acetate in heptane) to give (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate.

LCMS: 87%, RT=2.19 min., (M+H)⁺=661 (method A).

### Synthesis of (R)-2-(((3R,4S,6R)-4-amino-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate

HCl (5-6 M in 2-propanol, 1 mL, 5.5 mmol) was added to a solution of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate (111 mg, 0.168 mmol) in 2-propanol (8 mL). After stirring for overnight, another quantity of HCl (5-6 M in 2-propanol, 1 mL, 5.5 mmol) was added and stirring was continued overnight. Then, the mixture was diluted with saturated aqueous NaHCO₃ (30 mL) and dichloromethane (30 mL) and stirred vigorously for 30 minutes. The layers were separated and the aqueous phase was extracted with dichloromethane (20 mL). The combined organics were dried over Na₂SO₄ and evaporated under reduced pressure to give crude (R)-2-(((3R,4S,6R)-4-amino-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate (101 mg) as a colorless glass-like solid that was used as such.

LCMS: 80%, RT=1.67 min., (M+H)⁺=561 and 15%, RT=1.72 min., (M+H)⁺=465 (product of next step) (method A).

### Synthesis of ((S)-1-(4-fluorophenyl)-3,4-dihydroisoquinolin-2(1H)-yl)((3R,4aR,7R,8aS)-3-(trifluoromethyl)octahydropyrano[3,4-b][1,4]oxazin-7-yl)methanone (Compound 5474)

Triethylamine (0.099 mL, 0.714 mmol) was added to a solution of crude (R)-2-(((3R,4S,6R)-4-amino-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate (101 mg, 0.168 mmol) in dichloromethane (4 mL) and the mixture was warmed to 40° C. After 8 hours, another quantity of triethylamine (0.248 mL, 1.784 mmol) was added and stirring was continued at 30° C. over the weekend. Then, the mixture was concentrated under reduced pressure. The residue was purified by flash column chromatography (silica, 0 to 2.5% methanol in dichloromethane). The product was dissolved in methanol, brought onto an SCX-2 column (2 g), and eluted with methanol (5 column volumes). Next, the column was eluted with ammonia in methanol (1 M). The basic fraction was concentrated to dryness under reduced pressure. The residue was lyophilized from a mixture of acetonitrile and water (1:1, 4 mL) to give ((S)-1-(4-fluorophenyl)-3,4-dihydroisoquinolin-2(1H)-yl)((3R,4aR,7R,8aS)-3-(trifluoromethyl)octahydropyrano[3,4-b][1,4]oxazin-7-yl)methanone (Compound 5474).

LCMS: 99%, RT=1.23 min., (M+H)⁺=465 (method P).

### Example 154

### ((S)-1-(4-fluorophenyl)-3,4-dihydroisoquinolin-2(1H)-yl)((3S,4aR,7R,8aS)-3-(trifluoromethyl)octahydropyrano[3,4-b][1,4]oxazin-7-yl)methanone (Compound 5473)

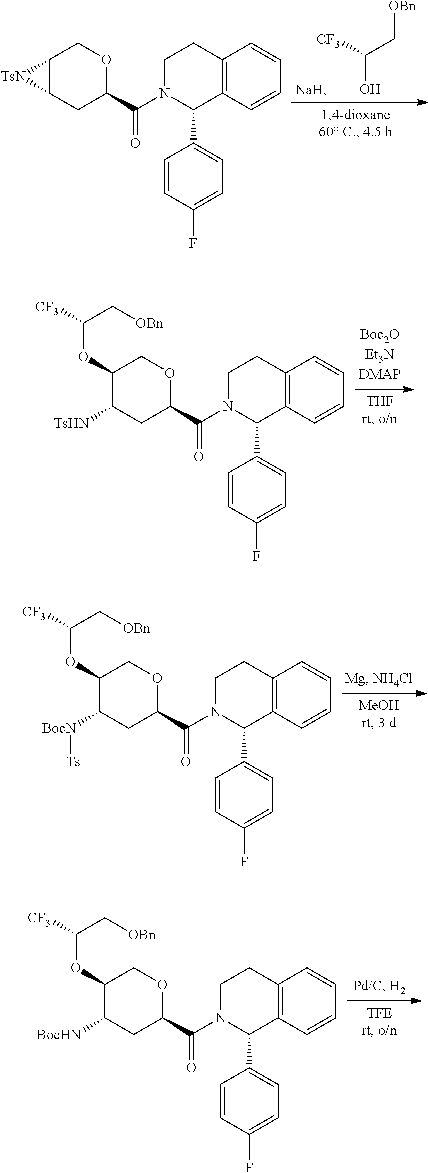

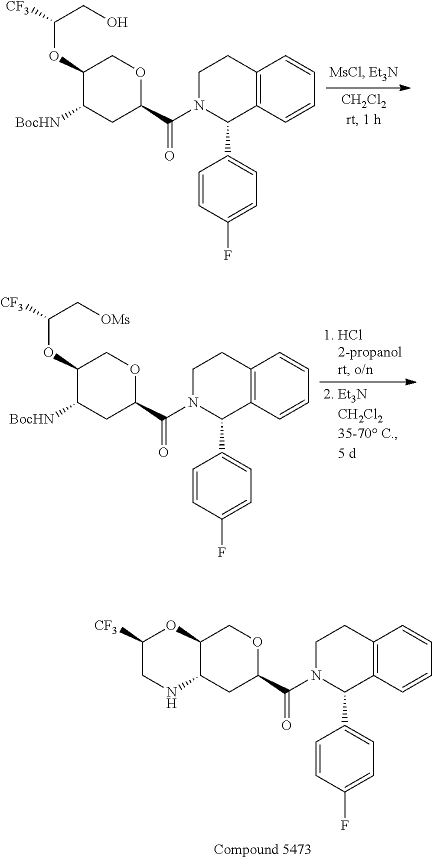 [SMILES: CC(C)(C)OC(=O)N[C@H]1C[C@H](C(=O)N2CCc3ccccc3[C@@H]2c2ccc(F)cc2)OC[C@@H]1O[C@@H](CO)C(F)(F)F ; CC(C)(C)OC(=O)N[C@H]1C[C@H](C(=O)N2CCc3ccccc3[C@@H]2c2ccc(F)cc2)OC[C@@H]1O[C@@H](COS(C)(=O)=O)C(F)(F)F ; O=C([C@H]1C[C@@H]2NC[C@@H](C(F)(F)F)O[C@H]2CO1)N1CCc2ccccc2[C@@H]1c1ccc(F)cc1]

### Synthesis of N-((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide

Under an argon atmosphere, sodium hydride (60% dispersion in mineral oil, 59.2 mg, 1.480 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 500 mg, 0.987 mmol) and (S)-3-(benzyloxy)-1,1,1-trifluoropropan-2-ol (652 mg, 2.96 mmol) in 1,4-dioxane (20 mL) and the mixture was heated to 60° C. After stirring for 4.5 hours, the mixture was allowed to cool to room temperature and stirred overnight. Then, the mixture was diluted with saturated aqueous NH₄Cl (35 mL) and water (10 mL) and extracted with ethyl acetate (35+20 mL). The combined organics were dried over Na₂SO₄ and evaporated under reduced pressure. The residue was purified by flash column chromatography (silica, 0 to 45% ethyl acetate in heptane) to give N-((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide.

LCMS: 89%, RT=2.49 min., (M+H)⁺=727 (method A).

### Synthesis of tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate

Triethylamine (0.163 mL, 1.170 mmol), di-tert-butyl dicarbonate (0.255 mg, 1.170 mmol)), and 4-dimethylaminopyridine (7.15 mg, 0.059 mmol) were added to a solution of N-((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)-4-methylbenzenesulfonamide (425 mg, 0.585 mmol) in tetrahydrofuran (dry, 8 mL). After stirring overnight, the mixture was concentrated under reduced pressure. The residue was purified by flash column chromatography (silica, 0 to 50% ethyl acetate in heptane) to give tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate.

LCMS: 89%, RT=2.49 min., (M+H)⁺=827 (method A).

### Synthesis of tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate

Ammonium chloride (310 mg, 5.79 mmol) and magnesium (704 mg, 29.0 mmol) were added to a solution of tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (480 mg, 0.579 mmol) in methanol (15 mL). After stirring for 4 hours, an extra quantity of magnesium (352 mg, 14.5 mmol) was added and stirring was continued over the weekend. Then, the mixture was diluted with saturated aqueous NH₄Cl (40 mL), stirred for 10 minutes, and extracted with dichloromethane (2×40 mL). The combined organics were dried over Na₂SO₄ and evaporated under reduced pressure. The residue was purified by flash column chromatography (silica, 0 to 50% ethyl acetate in heptane) to give tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate.

LCMS: 84%, RT=2.36 min., (M+H)⁺=673 (method A).

### Synthesis of tert-butyl ((2R,4S,5R)-2-((S)-1-(4-fluorophenyl)-1,2,3,4-tetrahydroisoquinoline-2-carbonyl)-5-(((S)-1,1,1-trifluoro-3-hydroxypropan-2-yl)oxy)tetrahydro-2H-pyran-4-yl)carbamate

A solution of tert-butyl ((2R,4S,5R)-5-(((S)-3-(benzyloxy)-1,1,1-trifluoropropan-2-yl)oxy)-2-((S)-1-(4-fluorophenyl)-1,2,3,4-tetrahydroisoquinoline-2-carbonyl)tetrahydro-2H-pyran-4-yl)carbamate (319 mg, 0.474 mmol) in 2,2,2-trifluoroethanol (7.5 mL) was hydrogenated in the presence of palladium (10 wt % on carbon, containing 50% water, 126 mg, 0.059 mmol) at atmospheric hydrogen pressure. After stirring overnight, the mixture was filtered over a 0.45 μm nylon filter and the filtrate was evaporated under reduced pressure. The residue was purified by flash column chromatography (silica, 0 to 50% ethyl acetate in heptane) to give tert-butyl ((2R,4S,5R)-2-((S)-1-(4-fluorophenyl)-1,2,3,4-tetrahydroisoquinoline-2-carbonyl)-5-(((S)-1,1,1-trifluoro-3-hydroxypropan-2-yl)oxy)tetrahydro-2H-pyran-4-yl)carbamate (73 mg) as a white solid.

LCMS: 62%, RT=2.17 min., (M+H)⁺=583 (method A).

### Synthesis of (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate

Triethylamine (0.052 mL, 0.376 mmol) and methanesulfonyl chloride (0.020 mL, 0.251 mmol) were added to a solution of tert-butyl ((2R,4S,5R)-2-((S)-1-(4-fluorophenyl)-1,2,3,4-tetrahydroisoquinoline-2-carbonyl)-5-(((S)-1,1,1-trifluoro-3-hydroxypropan-2-yl)oxy)tetrahydro-2H-pyran-4-yl)carbamate (73 mg, 0.125 mmol) in dichloromethane (3 mL). After stirring for 1 hour, the mixture was diluted with saturated aqueous NaHCO₃ (4 mL) and stirred for 15 minutes. The layers were separated over a phase separation filter and the organic filtrate was evaporated under reduced pressure. The residue was purified by flash column chromatography (silica, 10 to 60% ethyl acetate in heptane) to give (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate.

LCMS: 91%, RT=2.21 min., (M+H)⁺=661 (method A).

### Synthesis of ((S)-1-(4-fluorophenyl)-3,4-dihydroisoquinolin-2(1H)-yl)((3S,4aR,7R,8aS)-3-(trifluoromethyl)octahydropyrano[3,4-b][1,4]oxazin-7-yl)methanone (Compound 5473)

HCl (5-6 M in 2-propanol, 0.5 mL, 2.75 mmol) was added to a solution of (S)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)-3,3,3-trifluoropropyl methanesulfonate (45 mg, 0.068 mmol) in 2-propanol (1 mL). After stirring for overnight, the mixture was diluted with saturated aqueous NaHCO₃ and dichloromethane and stirred vigorously for 15 minutes. The organic phase was passed through a hydrophobic frit and evaporated under reduced pressure. In a reaction vial with cap, the residue was dissolved in dichloromethane, triethylamine (0.020 mL, 0.143 mmol) was added and the mixture was warmed to 35° C. After 2 days, another quantity of triethylamine (0.10 mL, 0.717 mmol) was added and stirring was continued at 70° C. for 3 hours and at 55° C. over the weekend. Then, the mixture was concentrated under reduced pressure. The residue was purified by acidic preparative MPLC (Linear Gradient: t=0 min 5% A; t=1 min 5% A; t=2 min 20% A; t=17 min 60% A; t=18 min 100%; t=23 min 100% A; detection: 220 nm). The combined product fractions were basified with saturated aqueous NaHCO₃ and the acetonitrile was removed under reduced pressure. The aqueous residue was extracted with dichloromethane (twice). The combined extracts were passed through a hydrophobic frit and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((3S,4aR,7R,8aS)-3-(trifluoromethyl)octahydropyrano[3,4-b][1,4]oxazin-7-yl)methanone (Compound 5473). LCMS: 99%, RT=1.27 min., (M+H)⁺=465 (method P)

### Example 155

### ((S)-1-(4-fluorophenyl)-3,4-dihydroisoquinolin-2(1H)-yl)((2R,4S,5R)-5-methoxy-4-((2-methoxyethyl)amino)tetrahydro-2H-pyran-2-yl)methanone (Compound 5569)

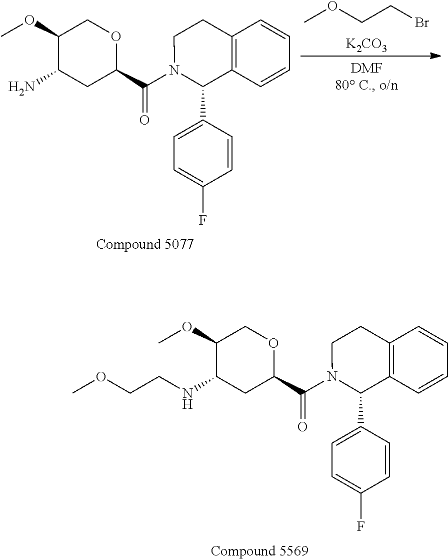

### Synthesis of ((S)-1-(4-fluorophenyl)-3,4-dihydroisoquinolin-2(1H)-yl)((2R,4S,5R)-5-methoxy-4-((2-methoxyethyl)amino)tetrahydro-2H-pyran-2-yl)methanone (Compound 5569)

Under argon atmosphere, potassium carbonate (575 mg, 4.16 mmol) and 2-bromoethyl methyl ether (0.198 mL, 2.081 mmol) were added to a solution of ((2R,4S,5R)-4-amino-5-methoxytetrahydro-2H-pyran-2-yl)((S)-1-(4-fluorophenyl)-3,4-dihydroisoquinolin-2(1H)-yl)methanone (see Compound 5077, 400 mg, 1.040 mmol) in N,N-dimethylformamide (dry, 8 mL) and the mixture was heated at 80° C. After stirring overnight, the mixture was diluted with ethyl acetate (30 mL) and water (15 mL). The aqueous layer was separated and extracted with ethyl acetate (20 mL). The combined organics were washed with brine (2×20 mL), dried over Na₂SO₄, and concentrated under reduced pressure. The residue was purified by acidic preparative MPLC (Linear Gradient: t=0 min 5% A; t=1 min 5% A; t=16 min 50% A; t=17 min 100%; t=22 min 100% A; detection: 215 nm) to give ((S)-1-(4-fluorophenyl)-3,4-dihydroisoquinolin-2(1H)-yl)((2R,4S,5R)-5-methoxy-4-((2-methoxyethyl)amino)tetrahydro-2H-pyran-2-yl)methanone (Compound 5569) after lyophilization from a mixture of acetonitrile and water (1:1).

LCMS: 98%, RT=1.66 min., (M+H)⁺=443 (method P).

### Example 156

### ((S)-1-(4-fluorophenyl)-3,4-dihydroisoquinolin-2(1H)-yl)((R)-morpholin-2-yl)methanone (COMPOUND 5405)

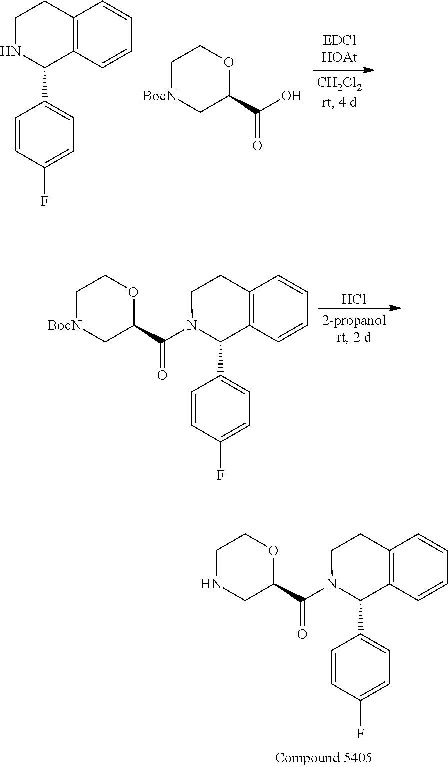

### Synthesis of tert-butyl (R)-2-((S)-1-(4-fluorophenyl)-1,2,3,4-tetrahydroisoquinoline-2-carbonyl)morpholine-4-carboxylate

N-(3-Dimethylaminopropyl)-N′-ethylcarbodiimide hydrochloride (139 mg, 0.726 mmol) and 1-hydroxy-7-azabenzotriazole (8.98 mg, 0.066 mmol) were added to a solution of (R)-4-(tert-butoxycarbonyl)morpholine-2-carboxylic acid (168 mg, 0.726 mmol) and (S)-1-(4-fluorophenyl)-1,2,3,4-tetrahydroisoquinoline (150 mg, 0.660 mmol) in dichloromethane (5 mL). After stirring for 4 days, the reaction mixture was diluted with saturated aqueous NaHCO₃. The organic layer was passed through a hydrophobic frit and evaporated under reduced pressure. The residue was purified by flash column chromatography (silica, 5 to 50% ethyl acetate in heptane) to give tert-butyl (R)-2-((S)-1-(4-fluorophenyl)-1,2,3,4-tetrahydroisoquinoline-2-carbonyl)morpholine-4-carboxylate.

LCMS: 99%, RT=2.17 min., (M+Na)⁺=463 (method A).

### Synthesis of ((S)-1-(4-fluorophenyl)-3,4-dihydroisoquinolin-2(1H)-yl)((R)-morpholin-2-yl)methanone (COMPOUND 5405)

HCl (5-6 M in 2-propanol, 1.667 mL, 9.17 mmol) was added to a solution of tert-butyl (R)-2-((S)-1-(4-fluorophenyl)-1,2,3,4-tetrahydroisoquinoline-2-carbonyl)morpholine-4-carboxylate (255 mg, 0.579 mmol) in 2-propanol (5 mL). After stirred for 2 days, the reaction mixture was diluted with saturated aqueous NaHCO₃ (15 mL) and dichloromethane and stirred for 15 minutes. The organic layer was passed through a hydrophobic frit and evaporated under reduced pressure. The residue was dissolved in methanol, brought onto an SCX-2 column (2 g), and eluted with methanol until neutral. Next, the column was eluted with ammonia in methanol (2 M). The basic fraction was concentrated to dryness under reduced pressure. The residue was lyophilized from a mixture of acetonitrile and water (1:1, 4 mL) to give ((S)-1-(4-fluorophenyl)-3,4-dihydroisoquinolin-2(1H)-yl)((R)-morpholin-2-yl)methanone (COMPOUND 5405).

LCMS: 99%, RT=1.03 min., (M+H)⁺=341 (method P).

### Example 157

### ((S)-1-(4-fluorophenyl)-3,4-dihydroisoquinolin-2(1H)-yl)((S)-morpholin-2-yl)methanone (COMPOUND 5406)

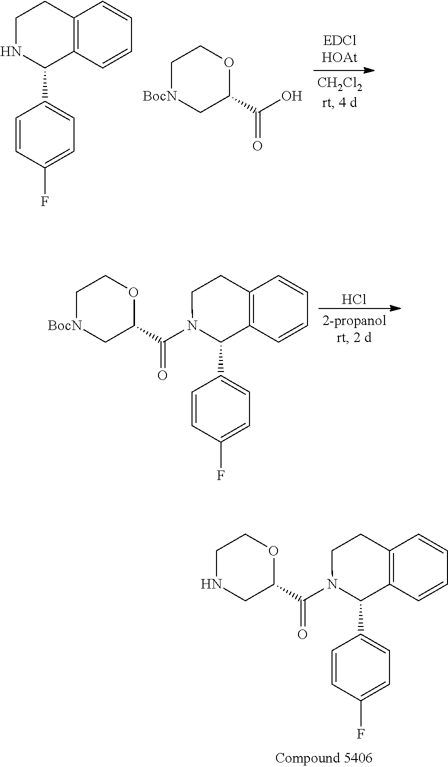

### Synthesis of tert-butyl (S)-2-((S)-1-(4-fluorophenyl)-1,2,3,4-tetrahydroisoquinoline-2-carbonyl)morpholine-4-carboxylate

N-(3-Dimethylaminopropyl)-N′-ethylcarbodiimide hydrochloride (139 mg, 0.726 mmol) and 1-hydroxy-7-azabenzotriazole (8.98 mg, 0.066 mmol) were added to a solution of (S)-4-(tert-butoxycarbonyl)morpholine-2-carboxylic acid (168 mg, 0.726 mmol) and (S)-1-(4-fluorophenyl)-1,2,3,4-tetrahydroisoquinoline (150 mg, 0.660 mmol) in dichloromethane (5 mL). After stirring for 4 days, the reaction mixture was diluted with saturated aqueous NaHCO₃. The organic layer was passed through a hydrophobic frit and evaporated under reduced pressure. The residue was purified by flash column chromatography (silica, 5 to 50% ethyl acetate in heptane) to give tert-butyl (S)-2-((S)-1-(4-fluorophenyl)-1,2,3,4-tetrahydroisoquinoline-2-carbonyl)morpholine-4-carboxylate.

LCMS: 99%, RT=2.15 min., (M+Na)⁺=463 (method A).

### Synthesis of ((S)-1-(4-fluorophenyl)-3,4-dihydroisoquinolin-2(1H)-yl)((S)-morpholin-2-yl)methanone (COMPOUND 5406)

HCl (5-6 M in 2-propanol, 1.667 mL, 9.17 mmol) was added to a solution of tert-butyl (S)-2-((S)-1-(4-fluorophenyl)-1,2,3,4-tetrahydroisoquinoline-2-carbonyl)morpholine-4-carboxylate (260 mg, 0.590 mmol) in 2-propanol (5 mL). After stirred for 2 days, the reaction mixture was diluted with saturated aqueous NaHCO₃ (15 mL) and dichloromethane and stirred for 15 minutes. The organic layer was passed through a hydrophobic frit and evaporated under reduced pressure. The residue was dissolved in methanol, brought onto an SCX-2 column (2 g), and eluted with methanol until neutral. Next, the column was eluted with ammonia in methanol (2 M). The basic fraction was concentrated to dryness under reduced pressure. The residue was lyophilized from a mixture of acetonitrile and water (1:1, 4 mL) to give ((S)-1-(4-fluorophenyl)-3,4-dihydroisoquinolin-2(1H)-yl)((S)-morpholin-2-yl)methanone (COMPOUND 5406).

LCMS: 99%, RT=1.03 min., (M+H)⁺=341 (method P).

### Example 158

### (S)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino)⁶-sulfanone (COMPOUND 5555)

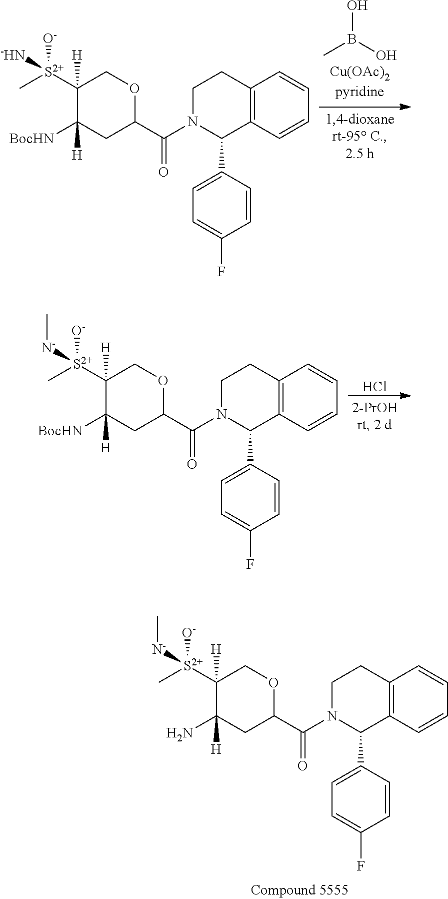

### Synthesis of tert-butyl ((2R,4S,5R)-5-((S)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate

Pyridine (0.025 mL, 0.311 mmol) was added to a suspension of tert-butyl ((2R,4S,5R)-2-((S)-1-(4-fluorophenyl)-1,2,3,4-tetrahydroisoquinoline-2-carbonyl)-5-((S)-S-methylsulfonimidoyl)tetrahydro-2H-pyran-4-yl)carbamate (see Compound 5547, 69 mg, 0.130 mmol), copper (II) acetate (39.6 mg, 0.218 mmol), and molecular sieves (4 Å, 100 mg) in 1,4-dioxane (dry, 2 mL). The reaction vessel was closed and stirred for 5 minutes. Then methylboronic acid (15.5 mg, 0.260 mmol) was added. The reaction vessel was sealed and heated to 95° C. After 2.5 hours, the reaction mixture was cooled to room temperature, diluted with dichloromethane (10 mL), and filtered through a 0.45 [symbol] m nylon filter. The filtrate was evaporated under reduced pressure. The residue was purified by flash column chromatography (silica, 30 to 100% ethyl acetate in heptane) to give tert-butyl ((2R,4S,5R)-5-((S)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate. Stereochemistry of the sulfoximine stereocenter is arbitrarily assigned.

LCMS: 97%, RT=1.91 min., (M+H)⁺=546 (method A).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino) ⁶-sulfanone (COMPOUND 5555)

HCl (5-6 M in 2-propanol, 1.0 mL, 5.0 mmol) was added to a solution of tert-butyl ((2R,4S,5R)-5-((S)-N, S-dimethylsulfonimidoyl)-2-((S)-1-(4-fluorophenyl)-1,2,3,4-tetrahydroisoquinoline-2-carbonyl)tetrahydro-2H-pyran-4-yl)carbamate (47 mg, 0.086 mmol) in 2-propanol (4 mL). After 2 days, the reaction mixture was basified with saturated aqueous NaHCO₃ and the 2-propanol was removed under reduced pressure. The residue was extracted with dichloromethane (20 mL). The organic layer was passed through a hydrophobic frit and evaporated under reduced pressure. The residue was purified by acidic preparative MPLC (Linear Gradient: t=0 min 5% A; t=1 min 5% A; t=16 min 40% A; t=17 min 100%; t=22 min 100% A; detection: 210 nm). The pooled product fractions were basified with saturated aqueous NaHCO₃ and the acetonitrile was removed under reduced pressure. The residue was extracted with dichloromethane. The organic layer was passed through a hydrophobic frit and evaporated under reduced pressure. The residue was filtered through a 0.25 Om nylon filter and lyophilized from a mixture of acetonitrile and water (1:1, 4 mL) to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino) [symbol] ⁶-sulfanone (COMPOUND 5555). Stereochemistry of the sulfoximine stereocenter is arbitrarily assigned.

LCMS: 98%, RT=1.06 min., (M+H)⁺=446 (method P).

### Example 159

### ((S)-1-(4-fluorophenyl)-3,4-dihydroisoquinolin-2(1H)-yl)((4R,4aR,7R,8aS)-4-imino-4-oxidooctahydro-2H-⁴-pyrano[3,4-b][1,4]thiazin-7-yl)methanone (COMPOUND 5553)

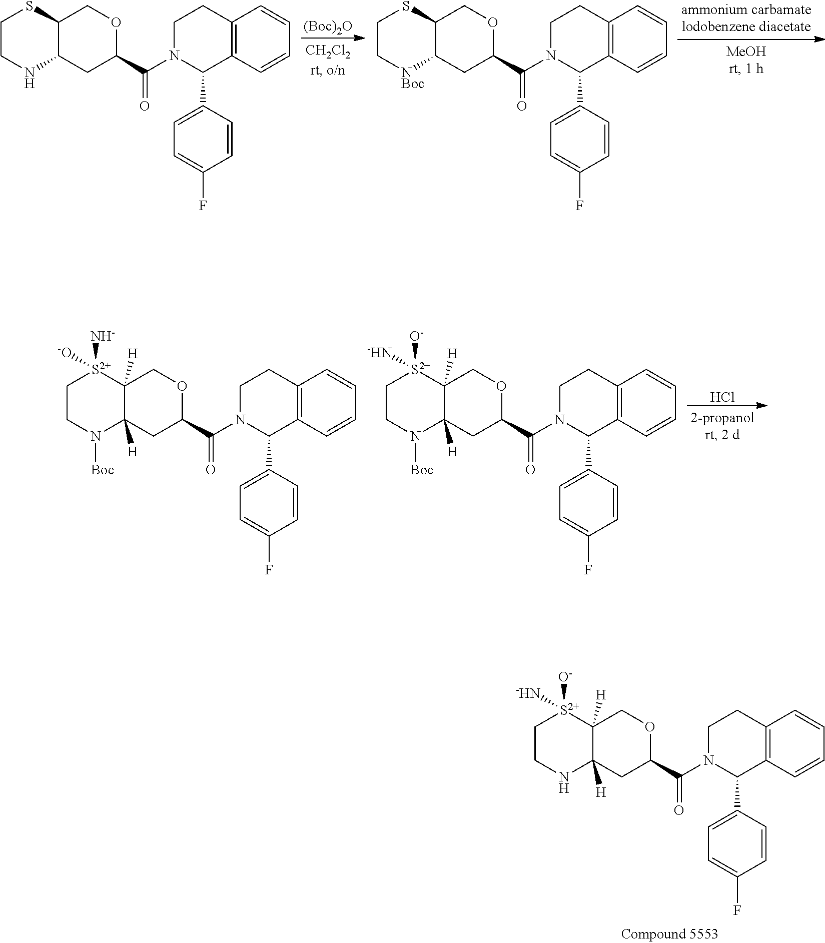

### Synthesis of tert-butyl (4aR,7R,8aS)-7-((S)-1-(4-fluorophenyl)-1,2,3,4-tetrahydroisoquinoline-2-carbonyl)hexahydropyrano[3,4-b][1,4]thiazine-1(5H)-carboxylate

Di-tert-butyl dicarbonate (135 mg, 0.619 mmol) was added to a solution of ((S)-1-(4-fluorophenyl)-3,4-dihydroisoquinolin-2(1H)-yl)((4aR,7R,8aS)-octahydropyrano[3,4-b][1,4]thiazin-7-yl)methanone (184 mg, 0.446 mmol) in dichloromethane (5 mL). After stirring overnight, the mixture was evaporated under reduced pressure. The residue was dissolved in a minimum amount of toluene and purified by flash column chromatography (silica, 5 to 40% ethyl acetate in heptane) to give The fractions containing the product was pooled and concentrated to give tert-butyl (4aR,7R,8aS)-7-((S)-1-(4-fluorophenyl)-1,2,3,4-tetrahydroisoquinoline-2-carbonyl)hexahydropyrano[3,4-b][1,4]thiazine-1(5H)-carboxylate.

LCMS: 99%, RT=2.29 min., (M+Na)⁺=535 (method A).

### Synthesis of tert-butyl (4S,4aR,7R,8aS)-7-((S)-1-(4-fluorophenyl)-1,2,3,4-tetrahydroisoquinoline-2-carbonyl)-4-iminohexahydro-2H-4⁴-pyrano[3,4-b][1,4]thiazine-1(5H)-carboxylate 4-oxide and tert-butyl (4R,4aR,7R,8aS)-7-((S)-1-(4-fluorophenyl)-1,2,3,4-tetrahydroisoquinoline-2-carbonyl)-4-iminohexahydro-2H-4⁴-pyrano[3,4-b][1,4]thiazine-1(5H)-carboxylate 4-oxide

Iodobenzene diacetate (267 mg, 0.827 mmol) was added to a solution of tert-butyl (4aR,7R,8aS)-7-((S)-1-(4-fluorophenyl)-1,2,3,4-tetrahydroisoquinoline-2-carbonyl)hexahydropyrano[3,4-b][1,4]thiazine-1(5H)-carboxylate (202 mg, 0.394 mmol) and ammonium carbamate (46.1 mg, 0.591 mmol) in methanol (2.5 mL). The reaction mixture was stirred under air and after 1 hour, the mixture was concentrated under reduced pressure. The residue was purified twice by flash column chromatography (silica, 0 to 10% (7M ammonia in methanol) in dichloromethane) and (silica, 25 to 100% ethyl acetate in heptane) to give tert-butyl (4S,4aR,7R,8aS)-7-((S)-1-(4-fluorophenyl)-1,2,3,4-tetrahydroisoquinoline-2-carbonyl)-4-iminohexahydro-2H-4 [symbol] ⁴-pyrano[3,4-b][1,4]thiazine-1(5H)-carboxylate 4-oxide as the first eluting isomer from the second column and tert-butyl (4R,4aR,7R,8aS)-7-((S)-1-(4-fluorophenyl)-1,2,3,4-tetrahydroisoquinoline-2-carbonyl)-4-iminohexahydro-2H-4 [symbol] ⁴-pyrano[3,4-b][1,4]thiazine-1(5H)-carboxylate 4-oxide as the second eluting isomer from the second column. Stereochemistry of the sulfoximine stereocenters is arbitrarily assigned.

First eluting isomer: LCMS: 99%, RT=2.03 min., (M+H)⁺=544 (method A). SFC: 99%, RT=4.94 min., (M+H)⁺=544 (method AZ).

Second eluting isomer: LCMS: 99%, RT=2.03 min., (M+H)⁺=544 (method A). SFC: 99%, RT=3.93 min., (M+H)⁺=544 (method AZ).

### Synthesis of ((S)-1-(4-fluorophenyl)-3,4-dihydroisoquinolin-2(1H)-yl)((4R,4aR,7R,8aS)-4-imino-4-oxidooctahydro-2H-4⁴-pyrano[3,4-b][1,4]thiazin-7-yl)methanone (COMPOUND 5553)

HCl (5 M in 2-propanol, 1.5 mL, 7.50 mmol) was added to a suspension of tert-butyl (4R,4aR,7R,8aS)-7-((S)-1-(4-fluorophenyl)-1,2,3,4-tetrahydroisoquinoline-2-carbonyl)-4-iminohexahydro-2H-4 [symbol] ⁴-pyrano[3,4-b][1,4]thiazine-1(5H)-carboxylate 4-oxide (64 mg, 0.118 mmol) in 2-propanol (6 mL). After 2 days, the reaction mixture was basified with saturated aqueous NaHCO₃ and the 2-propanol was removed under reduced pressure. The residue was extracted with dichloromethane. The organic layer was passed through a hydrophobic frit and evaporated under reduced pressure. The residue was purified by acidic preparative MPLC (Linear Gradient: t=0 min 5% A; t=1 min 5% A; t=3 min 15% A; t=18 min 40% A; t=19 min 100%; t=24 min 100% A; detection: 220 nm). The pooled product fractions were basified with saturated aqueous NaHCO₃ and the acetonitrile was removed under reduced pressure. The residue was extracted with dichloromethane. The organic layer was passed through a hydrophobic frit and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((4R,4aR,7R,8aS)-4-imino-4-oxidooctahydro-2H-4 [symbol] ⁴-pyrano[3,4-b][1,4]thiazin-7-yl)methanone (COMPOUND 5553). Stereochemistry of the sulfoximine stereocenter is arbitrarily assigned.

LCMS: 98%, RT=2.67 min., (M+H)⁺=444 (method AK). SFC: 100%, RT=3.51 min., (M+H)⁺=444 (method AS).

### Example 160

### ((S)-1-(4-fluorophenyl)-3,4-dihydroisoquinolin-2(1H)-yl)((4S,4aR,7R,8aS)-4-imino-4-oxidooctahydro-2H-4⁴-pyrano[3,4-b][1,4]thiazin-7-yl)methanone (COMPOUND 5554)

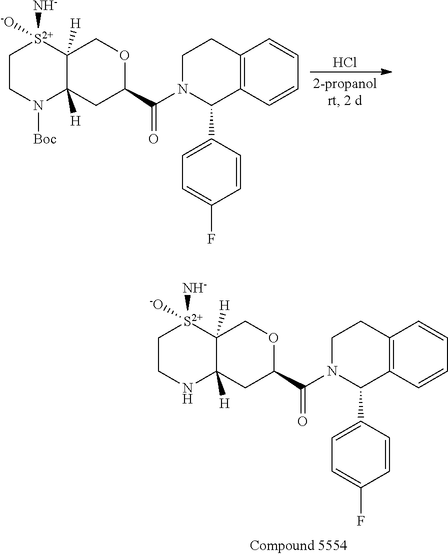

### Synthesis of ((S)-1-(4-fluorophenyl)-3,4-dihydroisoquinolin-2(1H)-yl)((4S,4aR,7R,8aS)-4-imino-4-oxidooctahydro-2H-4⁴-pyrano[3,4-b][1,4]thiazin-7-yl)methanone (COMPOUND 5554)

HCl (5 M in 2-propanol, 2.5 mL, 12.50 mmol) was added to a suspension of tert-butyl (4S,4aR,7R,8aS)-7-((S)-1-(4-fluorophenyl)-1,2,3,4-tetrahydroisoquinoline-2-carbonyl)-4-iminohexahydro-2H-4 [symbol] ⁴-pyrano[3,4-b][1,4]thiazine-1(5H)-carboxylate 4-oxide (64 mg, 0.118 mmol) in 2-propanol (10 mL). After 2 days, the reaction mixture was basified with saturated aqueous NaHCO₃ and the 2-propanol was removed under reduced pressure. The residue was extracted with dichloromethane. The organic layer was passed through a hydrophobic frit and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((4S,4aR,7R,8aS)-4-imino-4-oxidooctahydro-2H-4 [symbol] ⁴-pyrano[3,4-b][1,4]thiazin-7-yl)methanone (COMPOUND 5554). Stereochemistry of the sulfoximine stereocenter is arbitrarily assigned.

LCMS: 99%, RT=2.67 min., (M+H)⁺=444 (method AK). SFC: 99%, RT=3.15 min., (M+H)⁺=444 (method AS).

### Example 161

### (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)-⁶-sulfanone (COMPOUND 5557)

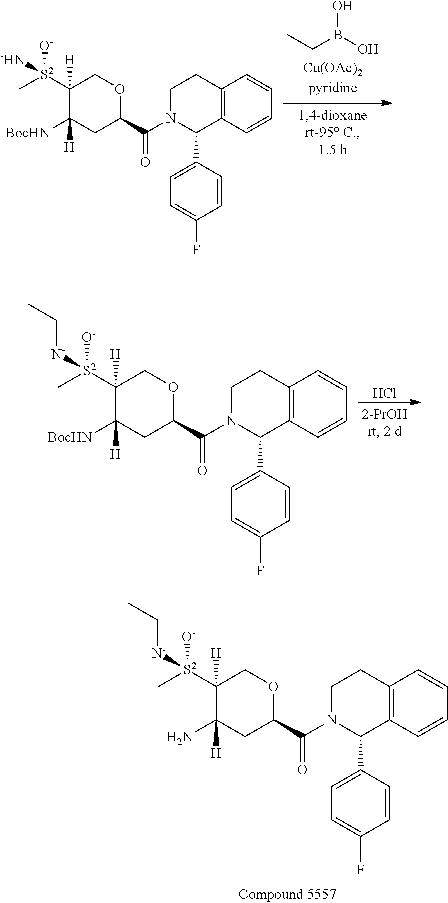

### Synthesis of tert-butyl ((2R,4S,5R)-5-((S)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate

Pyridine (0.157 mL, 1.947 mmol) was added to a suspension of tert-butyl ((2R,4S,5R)-2-((S)-1-(4-fluorophenyl)-1,2,3,4-tetrahydroisoquinoline-2-carbonyl)-5-((S)-S-methylsulfonimidoyl)tetrahydro-2H-pyran-4-yl)carbamate (see Compound 5547, 69 mg, 0.130 mmol), copper (II) acetate (40.1 mg, 0.221 mmol), and molecular sieves (4 Å, 100 mg) in 1,4-dioxane (dry, 2 mL). The reaction vessel was closed and stirred for 5 minutes. Then ethyl boronic acid (125 mg, 1.687 mmol) was added. The reaction vessel was sealed and heated to 95° C. After 1.5 hours, the reaction mixture was cooled to room temperature, diluted with dichloromethane (10 mL), and filtered through a 0.45 [symbol] m nylon filter. The filtrate was evaporated under reduced pressure. The residue was purified by flash column chromatography (silica, 40 to 100% ethyl acetate in heptane) to give tert-butyl ((2R,4S,5R)-5-((S)-N-ethyl-S-methylsulfonimidoyl)-2-((S)-1-(4-fluorophenyl)-1,2,3,4-tetrahydroisoquinoline-2-carbonyl)tetrahydro-2H-pyran-4-yl)carbamate after co-evaporation from methyl tert-butyl ether and pentane. Stereochemistry of the sulfoximine stereocenter is arbitrarily assigned.

LCMS: 99%, RT=1.94 min., (M+H)⁺=560 (method A).

### Synthesis of (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)⁶-sulfanone (COMPOUND 5557)

HCl (5 M in 2-propanol, 1.0 mL, 5.0 mmol) was added to a solution of tert-butyl ((2R,4S,5R)-5-((S)-N-ethyl-S-methylsulfonimidoyl)-2-((S)-1-(4-fluorophenyl)-1,2,3,4-tetrahydroisoquinoline-2-carbonyl)tetrahydro-2H-pyran-4-yl)carbamate (50 mg, 0.089 mmol) in 2-propanol (4 mL). After 2 days, the reaction mixture was basified with saturated aqueous NaHCO₃ and the 2-propanol was removed under reduced pressure. The residue was extracted with dichloromethane (10 mL). The organic layer was passed through a hydrophobic frit and evaporated under reduced pressure. The residue was purified by acidic preparative MPLC (Linear Gradient: t=0 min 10% A; t=2 min 10% A; t=17 min 50% A; t=18 min 100%; t=23 min 100% A; detection: 220 nm). The pooled product fractions were basified with saturated aqueous NaHCO₃ and the acetonitrile was removed under reduced pressure. The residue was extracted with dichloromethane (10 mL). The organic layer was passed through a hydrophobic frit and evaporated under reduced pressure. The residue was filtered through a 0.25 Om nylon filter and lyophilized from a mixture of acetonitrile and water (1:1, 4 mL) to give (S)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)- [symbol] ⁶-sulfanone (COMPOUND 5557). Stereochemistry of the sulfoximine stereocenter is arbitrarily assigned.

LCMS: 98%, RT=1.10 min., (M+H)⁺=460 (method P).

### Example 162

### (R)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino)-⁶-sulfanone (COMPOUND 5556)

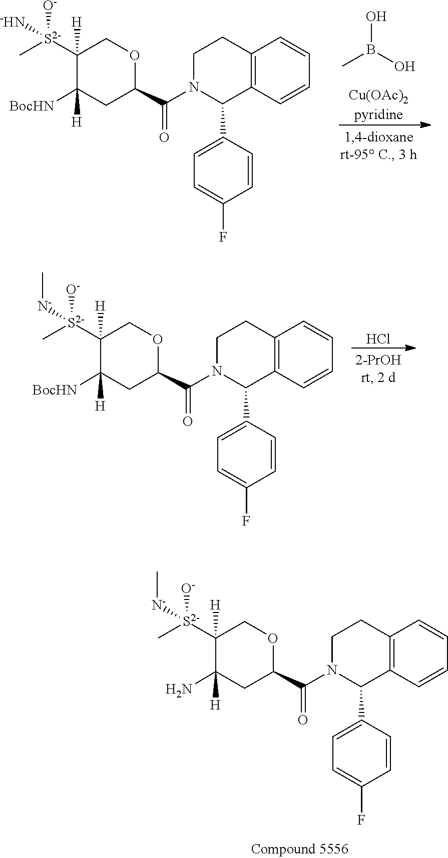

### Synthesis of tert-butyl ((2R,4S,5R)-5-((R)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate

Pyridine (0.032 mL, 0.398 mmol) was added to a suspension of tert-butyl ((2R,4S,5R)-2-((S)-1-(4-fluorophenyl)-1,2,3,4-tetrahydroisoquinoline-2-carbonyl)-5-((R)-S-methylsulfonimidoyl)tetrahydro-2H-pyran-4-yl)carbamate (see Compound 5547, 84.7 mg, 0.159 mmol), copper (II) acetate (49.2 mg, 0.271 mmol), and molecular sieves (4 Å, 100 mg) in 1,4-dioxane (dry, 2 mL). The reaction vessel was closed and stirred for 5 minutes. Then methyl boronic acid (22 mg, 0.368 mmol) was added. The reaction vessel was sealed and heated to 95° C. After 3 hours, the reaction mixture was cooled to room temperature, diluted with dichloromethane and a few drops of methanol, and filtered through a nylon 0.45 [symbol] m filter. The filtrate was evaporated under reduced pressure. The residue was purified by flash column chromatography (silica, 50 to 100% ethyl acetate in heptane) to give tert-butyl ((2R,4S,5R)-5-((R)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate. Stereochemistry of the sulfoximine stereocenter is arbitrarily assigned.

LCMS: 99%, RT=1.92 min., (M+H)⁺=546 (method A).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino)-⁶-sulfanone (COMPOUND 5556)

HCl (5-6 M in 2-propanol, 1.25 mL, 6.25 mmol) was added to a solution of tert-butyl ((2R,4S,5R)-5-((R)-N, S-dimethylsulfonimidoyl)-2-((S)-1-(4-fluorophenyl)-1,2,3,4-tetrahydroisoquinoline-2-carbonyl)tetrahydro-2H-pyran-4-yl)carbamate (58 mg, 0.106 mmol) in 2-propanol (5 mL). After 2 days, the reaction mixture was basified with saturated aqueous NaHCO₃ and the 2-propanol was removed under reduced pressure. The residue was extracted with dichloromethane (20 mL). The organic layer was passed through a hydrophobic frit and evaporated under reduced pressure. The residue was purified by acidic preparative MPLC (Linear Gradient: t=0 min 10% A; t=3 min 10% A; t=18 min 50% A; t=19 min 100%; t=24 min 100% A; detection: 210 nm). The pooled product fractions were basified with saturated aqueous NaHCO₃ and the acetonitrile was removed under reduced pressure. The residue was extracted with dichloromethane. The organic layer was passed through a hydrophobic frit and evaporated under reduced pressure. The residue was lyophilized from a mixture of acetonitrile and water (1:1, 4 mL) to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(methyl)(methylimino)- [symbol] ⁶-sulfanone (COMPOUND 5556). Stereochemistry of the sulfoximine stereocenter is arbitrarily assigned.

LCMS: 99%, RT=1.05 min., (M+H)⁺=446 (method P).

### Example 163

### (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)-⁶-sulfanone (COMPOUND 5558)

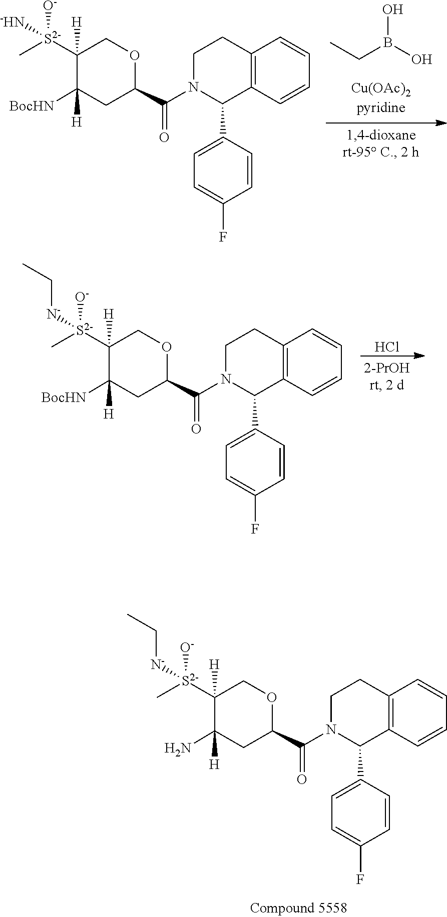

### Synthesis of tert-butyl ((2R,4S,5R)-5-((R)-N,S-dimethylsulfonimidoyl)-2-((S)-1-(4-fluorophenyl)-1,2,3,4-tetrahydroisoquinoline-2-carbonyl)tetrahydro-2H-pyran-4-yl)carbamate

Pyridine (0.276 mL, 0.341 mmol) was added to a suspension of tert-butyl ((2R,4S,5R)-2-((S)-1-(4-fluorophenyl)-1,2,3,4-tetrahydroisoquinoline-2-carbonyl)-5-((R)-S-methylsulfonimidoyl)tetrahydro-2H-pyran-4-yl)carbamate (see Compound 5547, 121 mg, 0.228 mmol), copper (II) acetate (70.3 mg, 0.387 mmol), and molecular sieves (4 Å, 200 mg) in 1,4-dioxane (dry, 3 mL). The reaction vessel was closed and stirred for 5 minutes. Then ethyl boronic acid (219 mg, 2.96 mmol) was added. The reaction vessel was sealed and heated to 95° C. After 2 hours, the reaction mixture was cooled to room temperature diluted with dichloromethane (20 mL) and a few drops of methanol, and filtered through a nylon 0.45 [symbol] m filter. The filtrate was evaporated under reduced pressure. The residue was purified by flash column chromatography (silica, 30 to 100% ethyl acetate in heptane) to give tert-butyl ((2R,4S,5R)-5-((R)-N-ethyl-S-methylsulfonimidoyl)-2-((S)-1-(4-fluorophenyl)-1,2,3,4-tetrahydroisoquinoline-2-carbonyl)tetrahydro-2H-pyran-4-yl)carbamate after co-evaporation from methyl tert-butyl ether. Stereochemistry of the sulfoximine stereocenter is arbitrarily assigned.

LCMS: 98%, RT=1.98 min., (M+H)⁺=560 (method A).

### Synthesis of (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)-⁶-sulfanone (COMPOUND 5558)

HCl (5 M in 2-propanol, 1.9 mL, 9.50 mmol) was added to a solution of tert-butyl ((2R,4S,5R)-5-((R)-N-ethyl-S-methylsulfonimidoyl)-2-((S)-1-(4-fluorophenyl)-1,2,3,4-tetrahydroisoquinoline-2-carbonyl)tetrahydro-2H-pyran-4-yl)carbamate (91 mg, 0.163 mmol) in 2-propanol (7.5 mL). After 2 days, the reaction mixture was basified with saturated aqueous NaHCO₃ and the 2-propanol was removed under reduced pressure. The residue was extracted with dichloromethane (10 mL). The organic layer was passed through a hydrophobic frit and evaporated under reduced pressure. The residue was purified by acidic preparative MPLC (Linear Gradient: t=0 min 10% A; t=2 min 10% A; t=17 min 50% A; t=18 min 100%; t=23 min 100% A; detection: 220 nm). The pooled product fractions were basified with saturated aqueous NaHCO₃ and the acetonitrile was removed under reduced pressure. The residue was extracted with dichloromethane (10 mL). The organic layer was passed through a hydrophobic frit and evaporated under reduced pressure. The residue was lyophilized from a mixture of acetonitrile and water (1:1, 4 mL) to give (R)-((3R,4S,6R)-4-amino-6-((S)-1-(4-fluorophenyl)-1,2,3,4-tetrahydroisoquinoline-2-carbonyl)tetrahydro-2H-pyran-3-yl)(ethylimino)(methyl)- [symbol] ⁶-sulfanone (COMPOUND 5558). Stereochemistry of the sulfoximine stereocenter is arbitrarily assigned.

LCMS: 96%, RT=1.09 min., (M+H)⁺=460 (method P).

### Example 164

### ((2R,4S,5R)-4-(dimethylamino)-5-methoxytetrahydro-2H-pyran-2-yl)((S)-1-(4-fluorophenyl)-3,4-dihydroisoquinolin-2(1H)-yl)methanone (COMPOUND 5575)

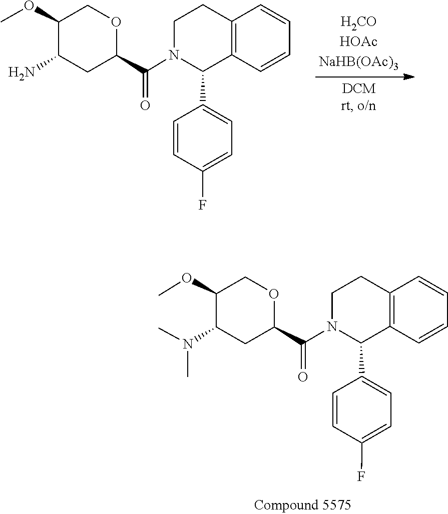 [SMILES: CO[C@H]1CO[C@@H](C(=O)N2CCc3ccccc3[C@@H]2c2ccc(F)cc2)C[C@@H]1N ; CO[C@H]1CO[C@@H](C(=O)N2CCc3ccccc3[C@@H]2c2ccc(F)cc2)C[C@@H]1N(C)C]

### Synthesis of ((2R,4S,5R)-4-(dimethylamino)-5-methoxytetrahydro-2H-pyran-2-yl)((S)-1-(4-fluorophenyl)-3,4-dihydroisoquinolin-2(1H)-yl)methanone (COMPOUND 5575)

Acetic acid (0.050 mL, 0.866 mmol) was added to a mixture of formaldehyde (37 wt % solution in water stabilized with 10-15% methanol, 0.325 mL, 4.33 mmol) and ((2R,4S,5R)-4-amino-5-methoxytetrahydro-2H-pyran-2-yl)((S)-1-(4-fluorophenyl)-3,4-dihydroisoquinolin-2(1H)-yl)methanone (see Compound 5077, 333 mg, 0.866 mmol) in dichloromethane (12 ml). After 5 minutes, sodium triacetoxyborohydride (459 mg, 2.165 mmol) was added and the mixture was stirred overnight. Then, the mixture was diluted with water (10 mL), stirred till all precipitates had dissolved, further diluted with saturated aqueous K₂CO₃, and stirred for 15 minutes. The organic layer was passed through a hydrophobic frit and evaporated under reduced pressure. The residue was purified by flash column chromatography (silica, 0 to 10% ammonia in methanol (7 M) in dichloromethane). The product was lyophilized from a mixture of acetonitril and water (1:1, 20 mL) to give ((2R,4S,5R)-4-(dimethylamino)-5-methoxytetrahydro-2H-pyran-2-yl)((S)-1-(4-fluorophenyl)-3,4-dihydroisoquinolin-2(1H)-yl)methanone (COMPOUND 5575).

LCMS: 98%, RT=1.99 min., (M+H)⁺=413 (method CA).

### Example 165

### ((S)-1-(4-fluorophenyl)-3,4-dihydroisoquinolin-2(1H)-yl)((3R,4aR,7R,8aS)-3-vinyloctahydropyrano[3,4-b][1,4]oxazin-7-yl)methanone (COMPOUND 5817)

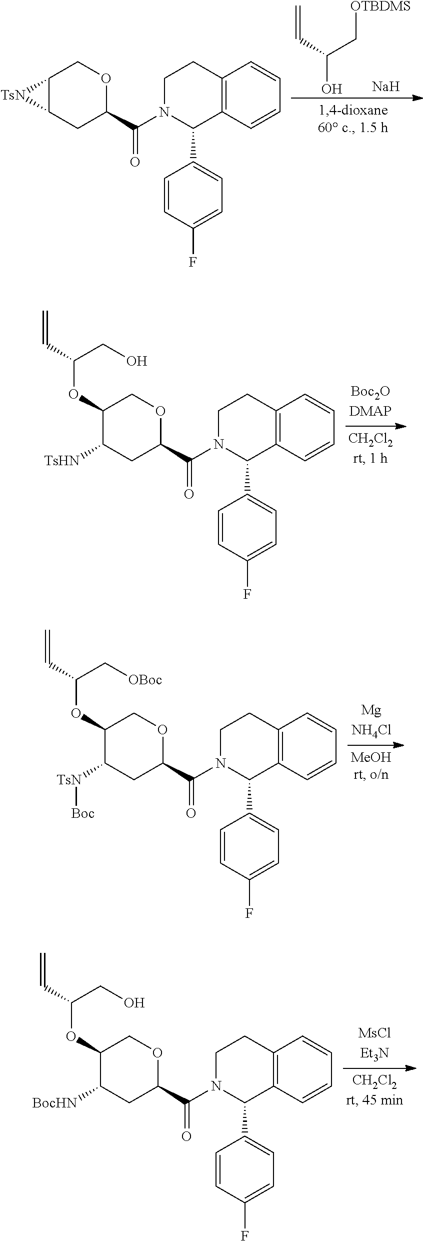

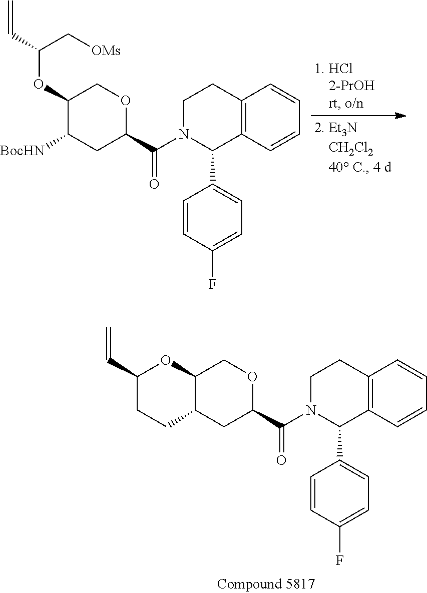 [SMILES: C=C[C@H](COS(C)(=O)=O)O[C@H]1CO[C@@H](C(=O)N2CCc3ccccc3[C@@H]2c2ccc(F)cc2)C[C@@H]1NC(=O)OC(C)(C)C ; C=C[C@@H]1CC[C@H]2C[C@H](C(=O)N3CCc4ccccc4[C@@H]3c3ccc(F)cc3)OC[C@@H]2O1]

### Synthesis of N-((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)-4-methylbenzenesulfonamide

Under argon atmosphere, sodium hydride (60% dispersion in mineral oil, 0.297 g, 7.43 mmol) was added to a suspension of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 2.51 g, 4.95 mmol) and (R)-1-((tert-butyldimethylsilyl)oxy)but-3-en-2-ol (1.504 g, 7.43 mmol) in 1,4-dioxane (100 mL) and the mixture was heated to 60° C. After 1.5 hours, the mixture was cooled to room temperature, diluted with saturated aqueous NH₄Cl and some water, and extracted with ethyl acetate (2×75 mL). The combined organics were dried over Na₂SO₄ and evaporated under reduced pressure. The residue was purified by flash column chromatography (silica, 10 to 66% ethyl acetate in heptane) and acidic preparative MPLC (Linear Gradient: t=0 min 5% A; t=1 min 5% A; t=2 min 30% A; t=17 min 70% A; t=18 min 100%; t=23 min 100% A; detection: 220 nm) to give N-((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)-4-methylbenzenesulfonamide.

LCMS: 99%, RT=2.12 min., (M+H)⁺=595 (method A).

### Synthesis of tert-butyl ((2R,4S,5R)-5-(((R)-1-((tert-butoxycarbonyl)oxy)but-3-en-2-yl)oxy)-2-((S)-1-(4-fluorophenyl)-1,2,3,4-tetrahydroisoquinoline-2-carbonyl)tetrahydro-2H-pyran-4-yl)(tosyl)carbamate

Di-tert-butyl dicarbonate (0.434 mg, 1.988 mmol) and 4-dimethylaminopyridine (24.28 mg, 0.199 mmol) were added to a solution of N-((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)-4-methylbenzenesulfonamide (591 mg, 0.994 mmol) in dichloromethane (30 mL). After stirring for 1 hour, the mixture was concentrated under reduced pressure. The residue was dissolved in methanol (15 mL) and potassium carbonate (275 mg, 1.988 mmol) was added. After stirring for 30 minutes, the mixture was diluted with water and extracted with dichloromethane (3×). The combined organic layers were passed through a hydrophobic frit and evaporated under reduced pressure. The residue was purified by flash column chromatography (silica, 5 to 50% ethyl acetate in heptane) to give tert-butyl ((2R,4S,5R)-5-(((R)-1-((tert-butoxycarbonyl)oxy)but-3-en-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (386 mg) as a white solid.

LCMS: 99%, RT=2.48 min., (M+Na)⁺=817 (method A).

### Synthesis of tert-butyl ((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)carbamate

Ammonium chloride (260 mg, 4.86 mmol) and magnesium (590 mg, 24.28 mmol) were added to a solution of tert-butyl ((2R,4S,5R)-5-(((R)-1-((tert-butoxycarbonyl)oxy)but-3-en-2-yl)oxy)-2-((S)-1-(4-fluorophenyl)-1,2,3,4-tetrahydroisoquinoline-2-carbonyl)tetrahydro-2H-pyran-4-yl)(tosyl)carbamate (386 mg, 0.486 mmol) in methanol (30 mL). The resulting suspension was stirred in a water bath overnight after which additional ammonium chloride (130 mg, 2.428 mmol) and magnesium (295 mg, 12.14 mmol) were added and stirring was continued overnight. Then, the mixture was diluted with saturated aqueous NH₄Cl, stirred for 15 minutes, and extracted with dichloromethane (3×). The combined organic layers were dried over Na₂SO₄ and evaporated under reduced pressure. The residue was purified by flash column chromatography (silica, 5 to 50% ethyl acetate in heptane) to give tert-butyl ((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)carbamate.

LCMS: 99%, RT=2.07 min., (M+H)⁺=541 (method A).

### Synthesis of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)but-3-en-1-yl methanesulfonate

Methanesulfonyl chloride (0.038 mL, 0.483 mmol) was added to a solution of tert-butyl ((2R,4S,5R)-2-((S)-1-(4-fluorophenyl)-1,2,3,4-tetrahydroisoquinoline-2-carbonyl)-5-(((R)-1-hydroxybut-3-en-2-yl)oxy)tetrahydro-2H-pyran-4-yl)carbamate (130.5 mg, 0.241 mmol) and triethylamine (0.101 mL, 0.724 mmol) in dichloromethane (5 mL). After stirring for 45 minutes, the mixture was diluted with saturated aqueous NaHCO₃ and stirred vigorously for 15 minutes. The organic layer was passed through a hydrophobic frit and evaporated under reduced pressure to give (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)but-3-en-1-yl methanesulfonate.

LCMS: 97%, RT=2.17 min., (M+H)⁺=619 (method A).

### Synthesis of ((S)-1-(4-fluorophenyl)-3,4-dihydroisoquinolin-2(1H)-yl)((3R,4aR,7R,8aS)-3-vinyloctahydropyrano[3,4-b][1,4]oxazin-7-yl)methanone (COMPOUND 5817)

HCl (5-6 M in 2-propanol, 1 mL, 5.50 mmol) was added to a solution of (R)-2-(((3R,4S,6R)-4-((tert-butoxycarbonyl)amino)-6-((S)-1-(4-fluorophenyl)-1,2,3,4-tetrahydroisoquinoline-2-carbonyl)tetrahydro-2H-pyran-3-yl)oxy)but-3-en-1-yl methanesulfonate (136 mg, 0.220 mmol) in 2-propanol (3 mL). After stirring overnight, the mixture was diluted with saturated aqueous NaHCO₃ and dichloromethane. The organic layer was passed through a hydrophobic frit and evaporated under reduced pressure. The residue was dissolved in dichloromethane (3 mL), triethylamine (0.092 mL, 0.659 mmol) was added, and the mixture was heated at 40° C. After 4 days, the mixture was concentrated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm) and the combined product fractions were lyophilized to give ((S)-1-(4-fluorophenyl)-3,4-dihydroisoquinolin-2(1H)-yl)((3R,4aR,7R,8aS)-3-vinyloctahydropyrano[3,4-b][1,4]oxazin-7-yl)methanone (COMPOUND 5817).

LCMS: 98%, RT=1.18 min., (M+H)⁺=423 (method P).

### Example 166

### ((3R,4aR,7R,8aS)-3-ethyloctahydropyrano[3,4-b][1,4]oxazin-7-yl)((S)-1-(4-fluorophenyl)-3,4-dihydroisoquinolin-2(1H)-yl)methanone (COMPOUND 5564)

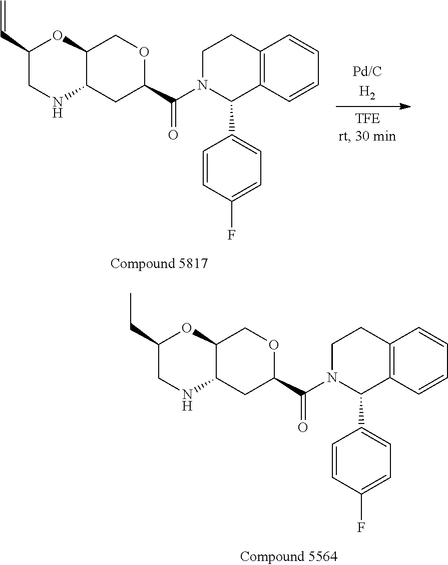 [SMILES: C=C[C@@H]1CN[C@H]2C[C@H](C(=O)N3CCc4ccccc4[C@@H]3c3ccc(F)cc3)OC[C@@H]2O1 ; CC[C@@H]1CN[C@H]2C[C@H](C(=O)N3CCc4ccccc4[C@@H]3c3ccc(F)cc3)OC[C@@H]2O1]

### Synthesis of ((3R,4aR,7R,8aS)-3-ethyloctahydropyrano[3,4-b][1,4]oxazin-7-yl)((S)-1-(4-fluorophenyl)-3,4-dihydroisoquinolin-2(1H)-yl)methanone (COMPOUND 5564)

A solution of ((S)-1-(4-fluorophenyl)-3,4-dihydroisoquinolin-2(1H)-yl)((3R,4aR,7R,8aS)-3-vinyloctahydropyrano[3,4-b][1,4]oxazin-7-yl)methanone (see Compound 5817, 47 mg, 0.111 mmol) in 2,2,2-trifluoroethanol (2 mL) was hydrogenated in the presence of palladium (10 wt % on carbon, containing 50% water, 37 mg, 0.017 mmol) at atmospheric hydrogen pressure. After stirring for 30 minutes, the mixture was filtered over a 0.45 μm nylon filter. The filtrate was brought onto an SCX-2 column and eluted with methanol (5 column volumes). Next, the column was eluted with ammonia in methanol (2 M). The basic fraction was concentrated to dryness under reduced pressure. The residue was lyophilized from a mixture of acetonitrile and water (1:1, 4 mL) to give ((3R,4aR,7R,8aS)-3-ethyloctahydropyrano[3,4-b][1,4]oxazin-7-yl)((S)-1-(4-fluorophenyl)-3,4-dihydroisoquinolin-2(1H)-yl)methanone (COMPOUND 5564).

LCMS: 97%, RT=1.20 min., (M+H)⁺=425 (method P).

### Example 167

### ((S)-1-(4-fluorophenyl)-3,4-dihydroisoquinolin-2(1H)-yl)((4aR,7R,8aS)-4-isopropyloctahydro-2H-pyrano[3,4-b]pyrazin-7-yl)methanone (COMPOUND 5602)

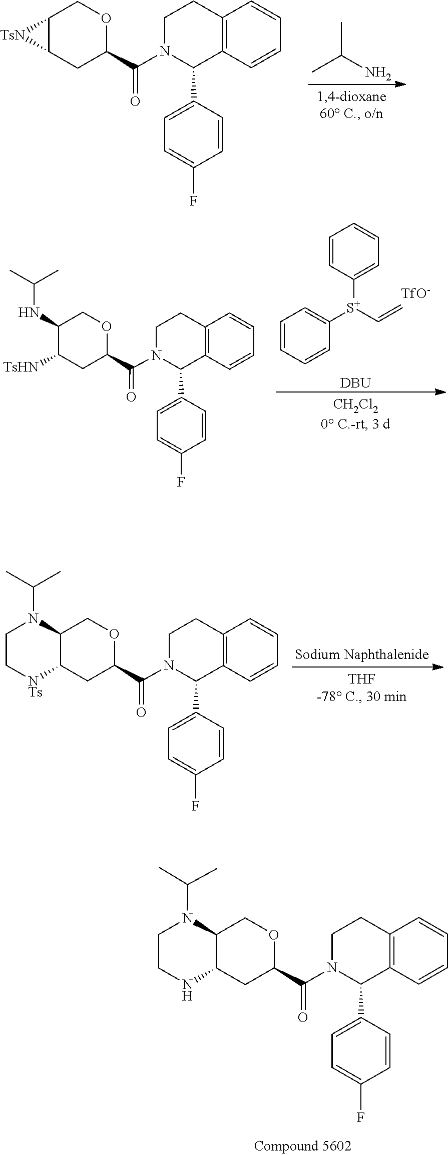

### Synthesis of N-((2R,4S,5R)-2-((S)-1-(4-fluorophenyl)-1,2,3,4-tetrahydroisoquinoline-2-carbonyl)-5-(isopropylamino)tetrahydro-2H-pyran-4-yl)-4-methylbenzenesulfonamide

Isopropylamine (0.168 mL, 1.974 mmol) was added to a solution of ((S)-1-(4-fluorophenyl)-3,4-dihydroisoquinolin-2(1H)-yl)((1S,4R,6S)-7-tosyl-3-oxa-7-azabicyclo[4.1.0]heptan-4-yl)methanone (see Compound 5520, 250 mg, 0.493 mmol) in 1,4-dioxane (4 mL) and the reaction mixture was heated to 60° C. After 5 hours, another portion of isopropylamine (0.084 mL, 0.987 mmol) was added and stirring was continued overnight. The reaction mixture was concentrated under reduced pressure to give N-((2R,4S,5R)-2-((S)-1-(4-fluorophenyl)-1,2,3,4-tetrahydroisoquinoline-2-carbonyl)-5-(isopropylamino)tetrahydro-2H-pyran-4-yl)-4-methylbenzenesulfonamide.

LCMS: 98%, RT=1.71 min., (M+H)⁺=566 (method A).

### Synthesis of ((S)-1-(4-fluorophenyl)-3,4-dihydroisoquinolin-2(1H)-yl)((4aR,7R,8aS)-4-isopropyl-1-tosyloctahydro-2H-pyrano[3,4-b]pyrazin-7-yl)methanone

Under argon atmosphere at 0° C., 1,8-diazabicyclo[5.4.0]undec-7-ene (0.171 mL, 1.141 mmol) was added to a solution of N-((2R,4S,5R)-2-((S)-1-(4-fluorophenyl)-1,2,3,4-tetrahydroisoquinoline-2-carbonyl)-5-(isopropylamino)tetrahydro-2H-pyran-4-yl)-4-methylbenzenesulfonamide (322.8 mg, 0.571 mmol) in dichloromethane (10 mL). After 10 minutes, a solution of ethenyldiphenylsulfanium trifluoromethanesulfonate (207 mg, 0.571 mmol) in dichloromethane (2 mL) was added dropwise. After 2 hours, the reaction mixture was warmed to room temperature and stirred for a further 2 hours. Then, another portion of 1,8-diazabicyclo[5.4.0]undec-7-ene (0.085 mL, 0.571 mmol) and a solution of ethenyldiphenylsulfanium trifluoromethanesulfonate (103 mg, 0.285 mmol) in dichloromethane (1 mL) were added. After stirring for 3 days, the mixture was diluted with saturated aqueous K₂CO₃ (5 mL) and water (5 mL) and stirred for 30 minutes. The aqueous layer was extracted with dichloromethane (2×5 mL). The combined organics were passed through a hydrophobic frit and evaporated under reduced pressure. The residue was purified by flash column chromatography (silica, 0 to 10% (7M ammonia in methanol) in dichloromethane) to give ((S)-1-(4-fluorophenyl)-3,4-dihydroisoquinolin-2(1H)-yl)((4aR,7R,8aS)-4-isopropyl-1-tosyloctahydro-2H-pyrano[3,4-b]pyrazin-7-yl)methanone.

LCMS: 98%, RT=1.84 min., (M+H)⁺=592 (method A).

### Synthesis of ((S)-1-(4-fluorophenyl)-3,4-dihydroisoquinolin-2(1H)-yl)((4aR,7R,8aS)-4-isopropyloctahydro-2H-pyrano[3,4-b]pyrazin-7-yl)methanone (COMPOUND 5602)

Under argon atmosphere at −78° C., a freshly prepared solution of sodium naphthalen-1-ide, (0.5 M in tetrahydrofuran (dry), 1.762 mL, 0.881 mmol) was added to a solution of ((S)-1-(4-fluorophenyl)-3,4-dihydroisoquinolin-2(1H)-yl)((4aR,7R,8aS)-4-isopropyl-1-tosyloctahydro-2H-pyrano[3,4-b]pyrazin-7-yl)methanone (173.8 mg, 0.294 mmol) in tetrahydrofuran (dry, 2 mL). After 30 minutes, the reaction mixture was partitioned between a mixture of saturated aqueous NH₄Cl (5 mL) and dichloromethane (10 mL). The aqueous layer was extracted with dichloromethane (2×5 mL) and the combined organics were passed through a hydrophobic frit and evaporated under reduced pressure. The residue was dissolved in methanol, brought onto an SCX-2 column (2 g), and eluted with methanol (5 column volumes). Next, the column was eluted with ammonia in methanol (1 M). The basic fraction was concentrated to dryness under reduced pressure. The residue was purified by preparative SFC (method BD). The product was lyophilized from a mixture of acetonitrile and water (1:1, 4 mL) to give ((S)-1-(4-fluorophenyl)-3,4-dihydroisoquinolin-2(1H)-yl)((4aR,7R,8aS)-4-isopropyloctahydro-2H-pyrano[3,4-b]pyrazin-7-yl)methanone (COMPOUND 5602).

LCMS: 97%, RT=1.87 min., (M+H)⁺=438 (method CA). SFC: 99%, RT=2.58 min., (M+H)⁺=438 (method V).

### Example 168

### ((R)-1-(2,4-difluorophenyl)-3,4-dihydroisoquinolin-2(1H)-yl)((R)-1,4-oxazepan-7-yl)methanone (COMPOUND 5263) and ((R)-1-(2,4-difluorophenyl)-3,4-dihydroisoquinolin-2(1H)-yl)((S)-1,4-oxazepan-7-yl)methanone (COMPOUND 5264)

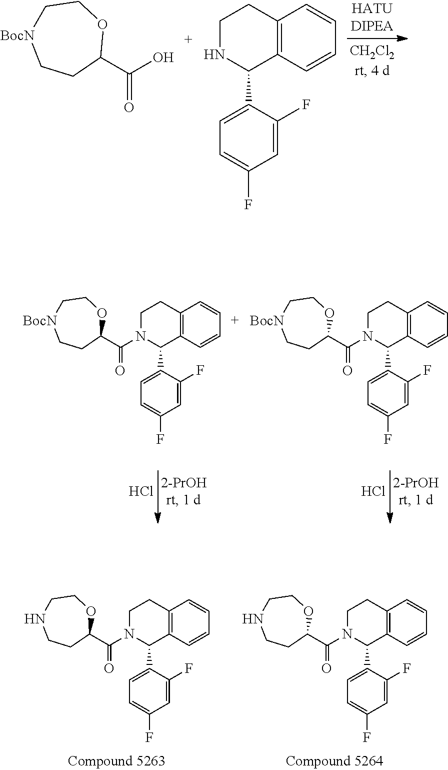

### Synthesis of tert-butyl (R)-7-((R)-1-(2,4-difluorophenyl)-1,2,3,4-tetrahydroisoquinoline-2-carbonyl)-1,4-oxazepane-4-carboxylate and tert-butyl (S)-7-((R)-1-(2,4-difluorophenyl)-1,2,3,4-tetrahydroisoquinoline-2-carbonyl)-1,4-oxazepane-4-carboxylate

1-[Bis(dimethylamino)methylene]-1H-1,2,3-triazolo[4,5-b]pyridinium 3-oxid hexafluorophosphate (159 mg, 0.419 mmol) and N,N-diisopropylethylamine (0.110 mL, 0.628 mmol) were added to a solution of 4-(tert-butoxycarbonyl)-1,4-oxazepane-7-carboxylic acid (118 mg, 0.419 mmol) in dichloromethane (2.5 mL). After 10 minutes, (R)-1-(2,4-difluorophenyl)-1,2,3,4-tetrahydroisoquinoline (103 mg, 0.419 mmol) was added and stirring was continued for 4 days. Then, the mixture was diluted with dichloromethane (20 mL), washed with aqueous HCl (1 M, 2×10 mL) and saturated aqueous NaHCO₃ (10 mL), dried over Na₂SO₄, and evaporated under reduced pressure. The residue was purified by flash column chromatography (silica, 0 to 40% ethyl acetate in heptane) and preparative SFC (method AE) to give tert-butyl (R)-7-((R)-1-(2,4-difluorophenyl)-1,2,3,4-tetrahydroisoquinoline-2-carbonyl)-1,4-oxazepane-4-carboxylate as the first eluting isomer on silica and tert-butyl (S)-7-((R)-1-(2,4-difluorophenyl)-1,2,3,4-tetrahydroisoquinoline-2-carbonyl)-1,4-oxazepane-4-carboxylate as the second eluting isomer on silica. Stereochemistry of the oxazepane ring assigned arbitrarily.

First eluting isomer: LCMS: 96%, RT=2.18 min., (M+Na)⁺=495 (method A). SFC: 99%, RT=2.85 min., (M+Na)⁺=495 (method AD).

Second eluting isomer: LCMS: 95%, RT=2.16 min., (M+H)⁺=473 (method A). SFC: 98%, RT=3.27 min., (M+Na)⁺=495 (method AD).

### Synthesis of ((R)-1-(2,4-difluorophenyl)-3,4-dihydroisoquinolin-2(1H)-yl)((R)-1,4-oxazepan-7-yl)methanone (COMPOUND 5263)

HCl (5 M in 2-propanol, 0.60 mL, 3.00 mmol) was added to a solution of tert-butyl (R)-7-((R)-1-(2,4-difluorophenyl)-1,2,3,4-tetrahydroisoquinoline-2-carbonyl)-1,4-oxazepane-4-carboxylate (50 mg, 0.106 mmol) in 2-propanol (1.8 mL). After 1 day, the reaction mixture was partitioned between dichloromethane (8 mL) and saturated aqueous Na₂CO₃ (8 mL). The aqueous layer was extracted with dichloromethane (8 mL). The combined organics were passed through a hydrophobic frit and evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product fractions were combined, basified with saturated aqueous Na₂CO₃ (4 mL), and extracted with ethyl acetate (4×10 mL). The combined organics were washed with brine (10 mL), dried over Na₂SO₄, and evaporated under reduced pressure. The residue was lyophilized from a mixture of acetonitrile and water (1:1, 4 mL) to give ((R)-1-(2,4-difluorophenyl)-3,4-dihydroisoquinolin-2(1/H)-yl)((R)-1,4-oxazepan-7-yl)methanone (COMPOUND 5263). Stereochemistry of the oxazepane ring assigned arbitrarily.

LCMS: 96%, RT=2.63 min., (M+H)⁺=373 (method AK).

### Synthesis of ((R)-1-(2,4-difluorophenyl)-3,4-dihydroisoquinolin-2(1H)-yl)((S)-1,4-oxazepan-7-yl)methanone (COMPOUND 5264)

HCl (5 M in 2-propanol, 0.60 mL, 3.00 mmol) was added to a solution of tert-butyl (S)-7-((R)-1-(2,4-difluorophenyl)-1,2,3,4-tetrahydroisoquinoline-2-carbonyl)-1,4-oxazepane-4-carboxylate (52.6 mg, 0.112 mmol) in 2-propanol (1.8 mL). After 1 day, the reaction mixture was partitioned between dichloromethane (8 mL) and saturated aqueous Na₂CO₃ (8 mL). The aqueous layer was extracted with dichloromethane (8 mL). The combined organics were passed through a hydrophobic frit and evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product fractions were combined, basified with saturated aqueous Na₂CO₃ (4 mL), and extracted with ethyl acetate (4×10 mL). The combined organics were washed with brine (10 mL), dried over Na₂SO₄, and evaporated under reduced pressure. The residue was lyophilized from a mixture of acetonitrile and water (1:1, 4 mL) to give ((R)-1-(2,4-difluorophenyl)-3,4-dihydroisoquinolin-2(1/H)-yl)((S)-1,4-oxazepan-7-yl)methanone (COMPOUND 5264). Stereochemistry of the oxazepane ring assigned arbitrarily.

LCMS: 99%, RT=2.58 min., (M+H)⁺=373 (method AK).

### Example 169

### ((S)-1-(4-fluorophenyl)-3,4-dihydroisoquinolin-2(1H)-yl)((4aS,7R,8aS)-4-(2-methoxyethyl)octahydro-2H-pyrano[3,4-b]pyrazin-7-yl)methanone (COMPOUND 5606)

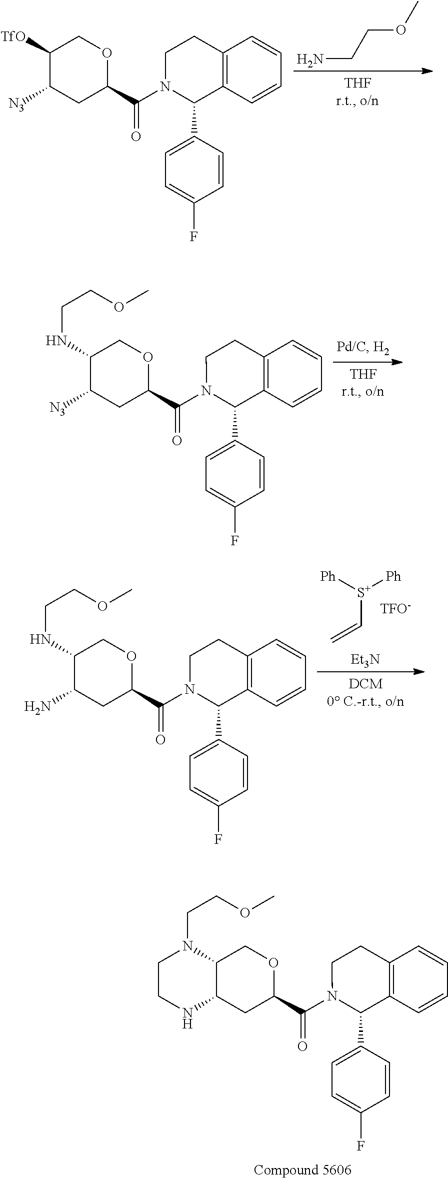

### Synthesis of ((2R,4S,5S)-4-azido-5-((2-methoxyethyl)amino)tetrahydro-2H-pyran-2-yl)((S)-1-(4-fluorophenyl)-3,4-dihydroisoquinolin-2(1H)-yl)methanone

2-Methoxyethylamine (0.651 mL, 7.48 mmol) was added to a solution of (3R,4S,6R)-4-azido-6-((S)-1-(4-fluorophenyl)-1,2,3,4-tetrahydroisoquinoline-2-carbonyl)tetrahydro-2H-pyran-3-yl trifluoromethanesulfonate (see Compound 5430, 304.2 mg, 0.576 mmol) in tetrahydrofuran (dry, 4.1 mL). After 3 days, the mixture was concentrated under reduced pressure. The residue was dissolved in methanol, brought onto an SCX-2 column (2 g), and eluted with methanol (5 column volumes). Next, the column was eluted with ammonia in methanol (1 M). The basic fraction was concentrated to dryness under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The combined product fractions were basified with saturated aqueous NaHCO₃ and extracted with dichloromethane. The combined extracts were dried over Na₂SO₄ and evaporated under reduced pressure to give ((2R,4S,5S)-4-azido-5-((2-methoxyethyl)amino)tetrahydro-2H-pyran-2-yl)((S)-1-(4-fluorophenyl)-3,4-dihydroisoquinolin-2(1H)-yl)methanone.

LCMS: 95%, RT=1.64 min., (M+H)⁺=454 (method A).

### Synthesis of ((2R,4S,5S)-4-amino-5-((2-methoxyethyl)amino)tetrahydro-2H-pyran-2-yl)((1S)-1-(4-fluorophenyl)-1,2,3,4-tetrahydronaphthalen-2-yl)methanone

A solution of ((2R,4S,5S)-4-azido-5-((2-methoxyethyl)amino)tetrahydro-2H-pyran-2-yl)((S)-1-(4-fluorophenyl)-3,4-dihydroisoquinolin-2(1H)-yl)methanone (134 mg, 0.295 mmol) in tetrahydrofuran (dry, 4.6 mL) was hydrogenated in the presence of palladium (10 wt % on carbon, containing 50% water, 110 mg, 0.052 mmol) at atmospheric hydrogen pressure. After stirring overnight, the mixture was diluted with ethanol (10 mL) and filtered over celite. The filtrate was evaporated under reduced pressure to give ((2R,4S,5S)-4-amino-5-((2-methoxyethyl)amino)tetrahydro-2H-pyran-2-yl)((S)-1-(4-fluorophenyl)-3,4-dihydroisoquinolin-2(1H)-yl)methanone (132 mg) as a faint yellow syrup that was used as such.

LCMS: 97%, RT=1.59 min., (M+H)⁺=428 (method A).

### Synthesis of ((S)-1-(4-fluorophenyl)-3,4-dihydroisoquinolin-2(1H)-yl)((4aS,7R,8aS)-4-(2-methoxyethyl)octahydro-2H-pyrano[3,4-b]pyrazin-7-yl)methanone (COMPOUND 5606)

Under argon atmosphere at 0° C., triethylamine (86 μL, 0.618 mmol) was added to a solution of ((2R,4S,5S)-4-amino-5-((2-methoxyethyl)amino)tetrahydro-2H-pyran-2-yl)((S)-1-(4-fluorophenyl)-3,4-dihydroisoquinolin-2(1H)-yl)methanone (132 mg, 0.309 mmol) in dichloromethane (20.5 mL). After 10 minutes, a solution of ethenyldiphenylsulfanium trifluoromethanesulfonate (75.0 mg, 0.207 mmol) in dichloromethane (10.20 mL) was added dropwise over 3 minutes. After 4 hours, the reaction mixture was warmed to room temperature and stirred overnight. Then, the mixture was diluted with dichloromethane (20 mL) and water (10 mL) and acidified with aqueous HCl (1 M). The layers were separated and the aqueous phase was extracted with dichloromethane (2×15 mL). Then, the aqueous layer was basified with saturated aqueous NaHCO₃ and extracted with dichloromethane (2×50 mL). The latter extracts were combined, washed with saturated aqueous NaHCO₃ (10 mL), dried over Na₂SO₄, and evaporated under reduced pressure. The residue was purified by basic preparative MPLC (Linear Gradient: t=0 min 5% A; t=1 min 5% A; t=2 min 20% A; t=17 min 60% A; t=18 min 100%; t=23 min 100% A; detection: 210 nm) to give ((S)-1-(4-fluorophenyl)-3,4-dihydroisoquinolin-2(1/H)-yl)((4aS,7R,8aS)-4-(2-methoxyethyl)octahydro-2H-pyrano[3,4-b]pyrazin-7-yl)methanone (COMPOUND 5606) after lyophilization.

LCMS: 98%, RT=1.91 min., (M+H)⁺=454 (method CA).

### Example 170

### ((S)-1-(4-fluorophenyl)-3,4-dihydroisoquinolin-2(1H)-yl)((4aR,7R,8aS)-1-methyl-4-(2,2,2-trifluoroethyl)octahydro-2H-pyrano[3,4-b]pyrazin-7-yl)methanone (COMPOUND 5735)

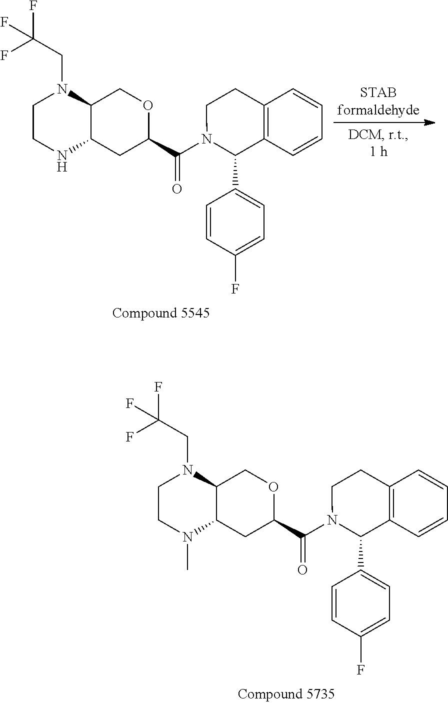 [SMILES: O=C([C@H]1C[C@@H]2NCCN(CC(F)(F)F)[C@H]2CO1)N1CCc2ccccc2[C@@H]1c1ccc(F)cc1 ; CN1CCN(CC(F)(F)F)[C@H]2CO[C@@H](C(=O)N3CCc4ccccc4[C@@H]3c3ccc(F)cc3)C[C@@H]21]

### Synthesis of ((S)-1-(4-fluorophenyl)-3,4-dihydroisoquinolin-2(1H)-yl)((4aR,7R,8aS)-1-methyl-4-(2,2,2-trifluoroethyl)octahydro-2H-pyrano[3,4-b]pyrazin-7-yl)methanone (COMPOUND 5735)

Formaldehyde (37 wt % solution in water, stabilized with 5-15% methanol, 5.82 μL, 0.077 mmol) and sodium triacetoxyborohydride (9.85 mg, 0.046 mmol) were added to a solution of ((S)-1-(4-fluorophenyl)-3,4-dihydroisoquinolin-2(1H)-yl)((4aR,7R,8aS)-4-(2,2,2-trifluoroethyl)octahydro-2H-pyrano[3,4-b]pyrazin-7-yl)methanone (see Compound 5545, 14.8 mg, 0.031 mmol) in dichloromethane (1 mL). After 2 hours, the reaction mixture was diluted by half with saturated K₂CO₃ solution (2 mL). The aqueous phase was extracted with dichloromethane (2×2 mL) and the combined organics were passed through a hydrophobic frit and evaporated under reduced pressure. The residue was lyophilized from a mixture of acetonitrile and water (1:1, 4 mL) to give ((S)-1-(4-fluorophenyl)-3,4-dihydroisoquinolin-2(1H)-yl)((4aR,7R,8aS)-1-methyl-4-(2,2,2-trifluoroethyl)octahydro-2H-pyrano[3,4-b]pyrazin-7-yl)methanone (COMPOUND 5735).

LCMS: 99%, RT=2.23 min., (M+H)⁺=492 (method CA).

### Example 171

Cellular Assays: Progranulin induction assay. To measure the efficacy of compounds, a progranulin induction cellular assay in mouse primary microglia (pMG), primary cortical neurons, and BV-2 cell lines is used. BV-2 cells are split the day before plating into a 96 well plate format at approximately 80%. Cells should be plated the day before and allowed for 1 hour attachment period and for 16 hour incubation. Levels of progranulin secreted into the cell culture medium or retained in the cell lysate can be quantified using an ELISA-based readout and measurement of secreted mouse PGRN in the medium was assessed by the methodology published by Ghidoni et al. 2012. Standard ELISA kits to measure PGRN are available from vendors such as Adipogen, R&D, and Biovendor.

Table B below presents the results of a progranulin induction assay as described above.

TABLE B

| Compound No. | EC₅₀ μM |
|---|---|
| 5000 | 0.41 |
| 5001 | 0.244 |
| 5002 | 0.278 |
| 5003 | 0.509 |
| 5004 | 0.164 |
| 5005 | 0.216 |
| 5006 | 0.143 |
| 5007 | 0.14 |
| 5008 | 0.369 |
| 5009 | 0.37 |
| 5010 | 0.573 |
| 5011 | 1.660 |
| 5012 | 0.125 |
| 5013 | 0.323 |
| 5014 | 0.501 |
| 5015 | 0.090 |
| 5016 | 0.199 |
| 5017 | 0.091 |
| 5018 | >10.0 |
| 5019 | 0.099 |
| 5020 | 1.060 |
| 5021 | 0.148 |
| 5022 | 4.160 |
| 5023 | 0.212 |
| 5024 | 0.749 |
| 5027 | 0.488 |
| 5028 | 0.042 |
| 5029 | 0.37 |
| 5030 | 0.348 |
| 5031 | 0.326 |
| 5032 | 0.213 |
| 5033 | 0.163 |
| 5034 | 0.185 |
| 5035 | 0.371 |
| 5036 | 0.356 |
| 5037 | 0.339 |
| 5038 | 0.5 |
| 5039 | 0.357 |
| 5040 | 0.173 |
| 5041 | 0.294 |
| 5042 | 0.079 |
| 5043 | 0.064 |
| 5044 | 0.163 |
| 5045 | 0.078 |
| 5046 | 0.062 |
| 5047 | 0.086 |
| 5048 | 0.575 |
| 5049 | 0.258 |
| 5050 | 0.35 |
| 5051 | 0.006 |
| 5052 | 0.018 |
| 5053 | 0.077 |
| 5054 | 0.13 |
| 5055 | 0.056 |
| 5056 | 0.127 |
| 5057 | 0.133 |
| 5058 | 0.29 |
| 5059 | 0.344 |
| 5060 | 5.850 |
| 5061 | 0.478 |
| 5062 | 0.049 |
| 5063 | 0.035 |
| 5064 | 0.102 |
| 5065 | 0.324 |
| 5066 | 0.948 |
| 5067 | 0.767 |
| 5068 | 0.376 |
| 5069 | 0.142 |
| 5070 | 0.153 |
| 5071 | 0.252 |
| 5072 | 0.081 |
| 5073 | 0.159 |
| 5074 | 0.23 |
| 5075 | 0.529 |
| 5076 | 0.321 |
| 5077 | 0.003 |
| 5078 | 0.001 |
| 5079 | >10.0 |
| 5080 | 0.166 |
| 5081 | 0.188 |
| 5082 | 0.044 |
| 5083 | 0.084 |
| 5084 | 0.18 |
| 5085 | 0.316 |
| 5086 | 0.194 |
| 5087 | 0.0421 |
| 5088 | 0.074 |
| 5089 | 0.062 |
| 5090 | 0.162 |
| 5091 | 0.408 |
| 5092 | 0.184 |
| 5207 | 0.182 |
| 5210 | 0.596 |
| 5211 | 0.209 |
| 5212 | 0.314 |
| 5213 | 0.401 |
| 5214 | 0.080 |
| 5215 | 0.140 |
| 5216 | 0.295 |
| 5217 | 0.226 |
| 5218 | 0.120 |
| 5219 | 0.006 |
| 5220 | 0.081 |
| 5235 | 0.372 |
| 5236 | 0.393 |
| 5237 | 0.665 |
| 5238 | 0.105 |
| 5239 | 0.316 |
| 5240 | 0.631 |
| 5243 | 0.200 |
| 5244 | 0.091 |
| 5245 | 0.350 |
| 5246 | 0.100 |
| 5247 | 0.095 |
| 5248 | 0.032 |
| 5250 | 0.095 |
| 5251 | 0.188 |
| 5255 | 0.030 |
| 5256 | 0.100 |
| 5257 | 0.022 |
| 5258 | 0.062 |
| 5259 | 0.046 |
| 5261 | 0.079 |
| 5262 | 0.044 |
| 5263 | 0.078 |
| 5264 | 0.048 |
| 5266 | 0.036 |
| 5267 | 0.0001 |
| 5405 | 0.736 |
| 5406 | 0.715 |
| 5418 | 0.352 |
| 5420 | >10 |
| 5421 | 0.843 |
| 5425 | 0.186 |
| 5426 | 0.441 |
| 5427 | 0.096 |
| 5428 | >10 |
| 5430 | >0.300 |
| 5431 | 0.029 |
| 5432 | 0.113 |
| 5443 | 0.151 |
| 5447 | 0.659 |
| 5448 | 0.044 |
| 5461 | 0.010 |
| 5462 | 0.003 |
| 5471 | 0.109 |
| 5472 | 0.013 |
| 5473 | 0.621 |
| 5474 | 1.510 |
| 5475 | 0.001 |
| 5476 | 0.001 |
| 5500 | 0.190 |
| 5501 | 3.860 |
| 5502 | 0.001 |
| 5503 | 0.005 |
| 5504 | 0.009 |
| 5505 | 0.410 |
| 5506 | 0.398 |
| 5507 | 0.001 |
| 5508 | 0.030 |
| 5509 | 0.001 |
| 5510 | 0.002 |
| 5511 | 0.0002 |
| 5512 | 0.005 |
| 5513 | 0.030 |
| 5514 | 0.024 |
| 5516 | 0.007 |
| 5517 | 0.012 |
| 5518 | 0.015 |
| 5519 | 0.041 |
| 5520 | 0.004 |
| 5521 | 0.008 |
| 5523 | 0.016 |
| 5524 | 0.016 |
| 5525 | 0.0003 |
| 5526 | 0.006 |
| 5527 | 0.274 |
| 5528 | 0.168 |
| 5529 | 1.560 |
| 5530 | 0.0003 |
| 5531 | 0.088 |
| 5532 | 1.680 |
| 5533 | 0.289 |
| 5534 | 0.015 |
| 5535 | 0.028 |
| 5536 | 0.150 |
| 5537 | 0.029 |
| 5538 | 0.064 |
| 5539 | 0.001 |
| 5540 | 0.210 |
| 5541 | 0.001 |
| 5542 | 4.390 |
| 5543 | 0.003 |
| 5544 | 0.019 |
| 5545 | 0.004 |
| 5546 | 0.030 |
| 5547 | 0.062 |
| 5548 | 0.071 |
| 5549 | 0.030 |
| 5550 | 0.017 |
| 5551 | 0.027 |
| 5552 | 0.011 |
| 5553 | 1.020 |
| 5554 | 4.860 |
| 5555 | 0.029 |
| 5556 | 0.070 |
| 5557 | 0.523 |
| 5558 | 0.312 |
| 5563 | 0.141 |
| 5564 | 0.004 |
| 5565 | >0.300 |
| 5566 | >0.300 |
| 5567 | 0.070 |
| 5568 | 0.025 |
| 5569 | >0.300 |
| 5570 | 0.232 |
| 5575 | 0.028 |
| 5577 | >0.300 |
| 5578 | 0.030 |
| 5579 | 0.050 |
| 5586 | 0.219 |
| 5587 | 0.194 |
| 5599 | 0.001 |
| 5600 | 0.021 |
| 5601 | 0.018 |
| 5603 | 0.008 |
| 5792 | >0.300 |
| 5793 | >0.300 |

In vivo Assays: Progranulin can be detected in tissue sample or biofluids including blood, plasma, brain, liver and cerebrospinal fluid (CSF) from different non-human animals including rat, mouse, dog, and cynomolgus monkeys. Progranulin concentration can be detected using one or more methods including an enzyme-linked immunosorbent assay (ELISA). Non-human animals including rat, mouse, dog, and cynomolgus monkeys are administered a compound as disclosed herein by one of several routes of administration including oral, subcutaneous, and intravenous routes of administration. Samples of tissue or biofluids are collected at specific times before and following compound administration. The amount of progranulin in a specific sample collected after compound administration is compared to the amount of progranulin detected in a sample of the same tissue or biofluid obtained prior to compound administration. Alternatively, the amount of progranulin can be compared to an equivalent sample taken from animals that did not receive the test compound. Test compounds can be administered one or more times. When compounds are administered more than one time, the number of doses and the intervals between doses can be adjusted to any combination. For example, a test compound can be administered a single time, a test compound can be administered 8 times at intervals of 12 hours, and a test compound can be administered 4 times at intervals of 24 hours. The ability of a compound to increase progranulin can be quantified in several ways. For example, the effect can be quantified as the progranulin concentration following compound administration, the effect can be quantified as the difference in progranulin concentration between samples collected post-administration and pre-administration, the effect can be quantified as the ratio or percent of progranulin in the post-administration sample relative to the pre-administration sample, and the effect can be quantified as the percent change in progranulin concentration relative to the pre-administration sample.

Treatment with the test compound increases the progranulin secretion relative to the control is at least about 110% (Activity level 1), at least about 130% (Activity level 2), at least about 150% (Activity level 3), at least about 180% (Activity level 4), at least about 200% (Activity level 5), at least about 250% (Activity level 6), or at least about 300% (Activity level 7).

Oral administration of compounds of the disclosure at 5 mg/kg resulted in increased progranulin secretion post dose relative to the control. Table C below presents the results of a progranulin secretion assay as described above.

TABLE C

| Compound No. | Activity Level |
|---|---|
| 5015 | 7 |
| 5042 | 6 |

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having a structure of Formula (I): 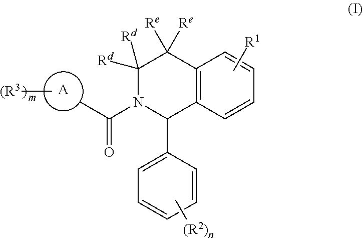
wherein
ring A is a 4- to 12-membered heterocycle comprising a ring O or S atom, further comprising 0-3 additional ring heteroatoms selected from O, N, and S;
R¹ is hydrogen, C₁₋₆alkyl, halo, C₁₋₃ haloalkyl, O—C₁₋₃ haloalkyl,C₁₋₃ alkylene-CN, C₁₋₃ alkylene-NR^{N}₂, C₀₋₆ alkylene-OR^{N}, C₀₋₆ alkylene- C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
each R² is independently halo;
each R³ is independently hydrogen, halo, C₁₋₆alkyl, C₁₋₆haloalkyl, C₀₋₆alkylene-OH, C₁₋₆alkoxy, C₁₋₆ haloalkoxy, C₁₋₆alkylene-O—C₁₋₆alkyl, C₀₋₆alkylene-NR^{a}R^{b}, S—C₁₋₆alkyl, C₂₋₆alkenyl, C(O)—C₁₋₆haloalkyl, SO₂—C₁₋₆ alkyl, S²⁺(O)—(NR^{a})—C₁₋₆alkyl, OR⁴, 5- to 8-membered heteroaryl comprising 1-4 ring N atoms, or 4- to 6-membered heterocycle comprising 1-4 ring heteroatoms selected from O, N, and S with at least 1 ring heteroatom being N, and the heteroaryl or heterocycle is optionally substituted with 1 or 2 substituents independently selected from halo, C₁₋₆alkyl, OH, and C₁₋₆alkoxy, or
two geminal R³ together with the atom to which they are attached form an oxo group, and
when ring A comprises a ring N atom, the N is substituted with R^{a}, and if ring A does not comprise a ring N atom, then at least one R³ is C₀₋₆alkylene-NR^{a}R^{b};
R^{a} and R^{b} are each independently hydrogen, C₁₋₆alkyl, C₁₋₆haloalkyl, C₁₋₆alkylene-OH, C₁₋₆alkylene-O—C₁₋₆alkyl, C(O)—C₁₋₆alkyl, C(O)—C₁₋₆haloalkyl, S(O)₂—C₁₋₆alkyl, S(O)₂—C₁₋₆haloalkyl; or
R^{a} and R^{b} together with the nitrogen to which they are attached form a 3- to 12-membered monocyclic or bicyclic heterocycle optionally further comprising 1-3 additional ring heteroatoms selected from O, N, and S;
R⁴ is C₂₋₆alkenyl, C₂₋₆alkynyl, C₀₋₆alkylene-C₃₋₆cycloalkyl, or C₀₋₆alkylene-C₆₋₁₀aryl;
each R^{d} is independently H or D;
each R^{e} is independently H, D, halo, OH, methyl, or methoxy, or
two geminal R^{e} together with the atom to which they are attached form an oxo group or a spiro C₃₋₅cycloalkyl;
m is 1-4; and
n is 0-2.

2. The compound or salt of claim 1, wherein ring A is a 4- to 6-membered heterocycle.

3. The compound or salt of claim 1 or 2, wherein ring A is a 6- to 8-membered heterocycle.

4. The compound or salt of any one of claims 1 to 3, wherein ring A comprises a ring O atom and 0-3 additional ring heteroatoms selected from O, N, and S.

5. The compound or salt of claim 4, wherein ring A comprises a tetrahydropyranyl ring.

6. The compound or salt of any one of claims 1 to 3, wherein ring A comprises a ring 0 and N atom.

7. The compound or salt of any one of claims 1 to 3, wherein ring A comprises a ring S atom and 0-3 additional ring heteroatoms selected from O, N, and S.

8. The compound or salt of any one of claims 1 to 3, wherein ring A is 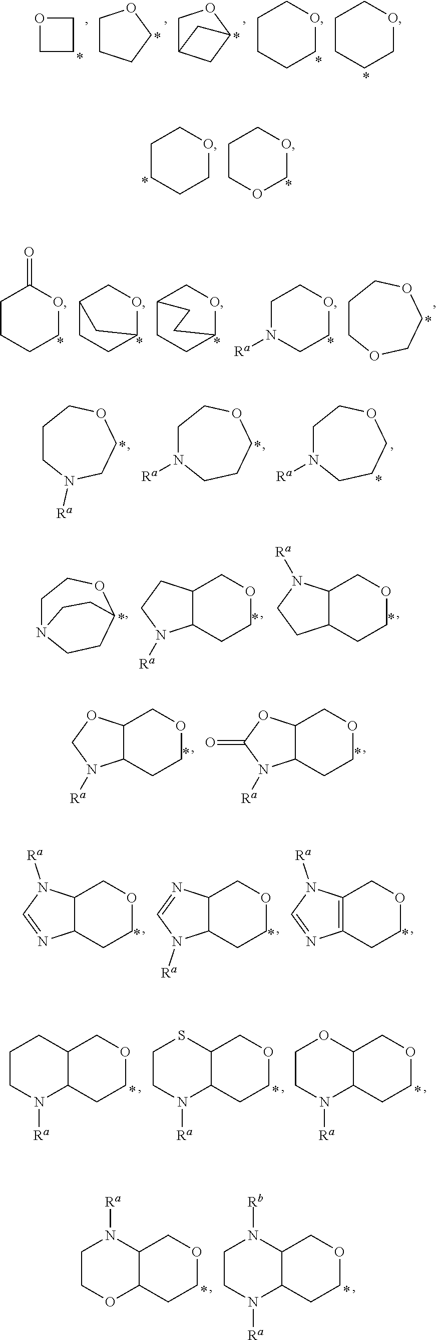 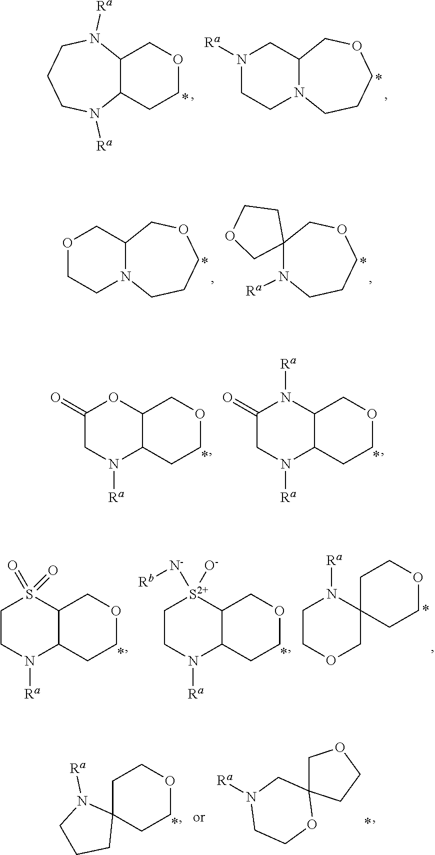
wherein * indicates the point of attachment of ring A to the adjacent carbonyl moiety of Formula I.

9. The compound or salt of claim 8, wherein ring A is

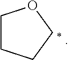

10. The compound or salt of claim 8, wherein ring A is

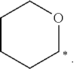

11. The compound or salt of claim 8, wherein ring A is

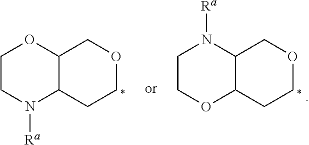

12. The compound or salt of any one of claims 1 to 11, wherein m is 1.

13. The compound or salt of claim 12, wherein R³ is hydrogen.

14. The compound or salt of any one of claims 1 to 11, wherein m is 3.

15. The compound or salt of any one of claims 1 to 11, wherein m is 2.

16. The compound or salt of claim 15, wherein ring A is

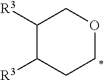

17. The compound or salt of any one of claims 1 to 12 and 14 to 16, wherein at least one R³ is C₀₋₆alkylene-NR^{a}R^{b}.

18. The compound or salt of claim 17, wherein at least one R³ is NH₂.

19. The compound or salt of claim 17, wherein at least one R³ is NHMe.

20. The compound or salt of any one of claims 1 to 12, and 14 to 19, wherein at least one R³ is halo.

21. The compound or salt of claim 20, wherein at least one R³ is F.

22. The compound or salt of any one of claims 1 to 12 and 14 to 21, wherein at least one R³ is C₁₋₆alkoxy

23. The compound or salt of claim 22, wherein at least one R³ is methoxy.

24. The compound or salt of claim 22, wherein at least one R³ is ethoxy.

25. The compound or salt of claim 21, wherein one R³ is F and one R³ is NH₂.

26. The compound or salt of claim 23, wherein one R³ is methoxy and one R³ is NH₂.

27. The compound or salt of claim 23, wherein one R³ is methoxy and one R³ is NHMe.

28. The compound or salt of claim 24, wherein one R³ is ethoxy and one R³ is NH₂.

29. The compound or salt of any one of claims 1 to 12, wherein R³ is S²⁺(O)—(NR^{a})—C₁₋₆alkyl.

30. The compound or salt of claim 29, wherein R³ is

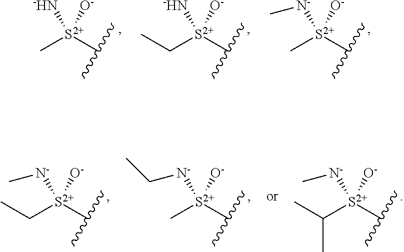

31. The compound or salt of any one of claims 1 to 12, wherein R³ is OR⁴.

32. The compound or salt of claim 31, wherein R⁴ is C₂₋₆alkynyl, C₀₋₆alkylene-C₃₋₆cycloalkyl, or C₀₋₆alkylene-C₆₋₁₀aryl.

33. The compound or salt of claim 33, wherein R³ is

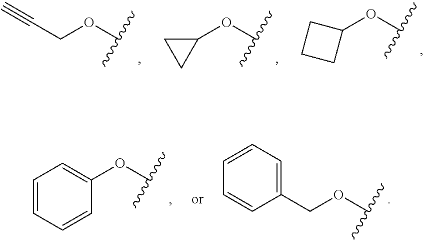

34. The compound or salt of any one of claims 1 to 33, wherein n is 0.

35. The compound or salt of any one of claims 1 to 33, wherein n is 1.

36. The compound or salt of any one of claims 1 to 33, wherein n is 2.

37. The compound or salt of any one of claims 1 to 28, 35 and 36, wherein R² is F or Cl.

38. The compound or salt of claim 37, wherein R² is F.

39. The compound or salt of any one of claims 1 to 38, wherein R¹ is halo.

40. The compound or salt of claim 39, wherein R¹ is F.

41. The compound or salt of any one of claims 1 to 38, wherein R¹ is hydrogen.

42. The compound or salt of any one of claims 1 to 41, wherein at least one R^{d} is H.

43. The compound or salt of claim 42, wherein each R^{d} is H.

44. The compound or salt of any one of claims 1 to 41, wherein at least wherein at least one R^{d} is D.

45. The compound or salt of any one of claims 1 to 44, wherein at least one R^{e} is H.

46. The compound or salt of claim 45, wherein each R^{e} is H.

47. The compound or salt of any one of claims 1 to 44, wherein at least one R^{e} is D.

48. The compound or salt of any one of claims 1 to 44, wherein each R^{e} is D.

49. The compound or salt of any one of claims 1 to 44, wherein at least one R^{e} is OH.

50. The compound or salt of any one of claims 1 to 44, wherein at least one R^{e} is halo.

51. The compound or salt of any one of claims 1 to 44, wherein at least one R^{e} is F.

52. The compound or salt of any one of claims 1 to 44, wherein each R^{e} is halo.

53. The compound or salt of any one of claims 1 to 44, wherein each R^{e} is F.

54. The compound or salt of any one of claims 1 to 44, wherein two geminal R^{e} together with the atom to which they are attached form an oxo group.

55. A compound, or pharmaceutically acceptable salt thereof, having a structure as shown in Table A.

56. The compound or salt of claim 55, selected from compound 5605, 5602, 5599, 5575, 5564, 5550, 5472, 5545, 5543, 5461, 5267, 5448, 5475, 5087, 5077, 5051, 5045, 5042, 5021, and 5012, and pharmaceutically acceptable salts thereof.

57. The compound or salt of claim 56, selected from compound 5599, 5564, 5472, 5077, and 5087, and pharmaceutically acceptable salts thereof.

58. The compound or salt of any one of claims 1 to 57 in the form of a salt.

59. A pharmaceutical composition comprising the compound or salt of any one of claims 1 to 58 and a pharmaceutically acceptable excipient.

60. Use of the compound or salt of any one of claims 1 to 58 as a medicament for the modulation of progranulin.

61. The use of claim 60, wherein progranulin secretion is increased.

62. A method of modulating progranulin in a subject in need thereof comprising administering to the subject the compound or salt of any one of claims 1 to 58 in an amount effective to increase the level of progranulin or granulin in the subject.

63. A method of treating a progranulin-associated disorder in a subject in need thereof comprising administering a therapeutically effective amount of the compound or salt of any one of claims 1 to 58 to the subject.

64. The method of claim 63, wherein the progranulin-associated disorder is Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), a lysosomal storage disease, nephropathy, a disease associated with inclusions and/or misfunction of C9orf72, TDP-43, FUS, UBQLN2, VCP, CHMP28, and/or MAPT, an acute neurological disorder, glioblastoma, or neuroblastoma.

65. The method of claim 64, wherein the Parkinson's disease is Parkinson's disease with GBA mutation.

66. The method of claim 64, wherein the lysosomal storage disease is Paget's disease, Gaucher's disease, Nieman's Pick disease, Tay-Sachs Disease, Fabry Disease, Pompes disease, or Naso-Hakula disease.

67. The method of claim 64, wherein the acute neurological disorder is stroke, cerebral hemorrhage, traumatic brain injury or head trauma.

68. The method of claim 64, wherein the progranulin-associated disorder is Frontotemporal dementia (FTD).

69. The method of claim 64, wherein the progranulin-associated disorder is Frontotemporal dementia-Granulin subtype (FTD-GRN).

70. A method of increasing lysosomal protein levels in a cell comprising contacting the cell with an effective amount of the compound or salt of any one of claims 1 to 58.

71. A method of increasing lysosomal protein levels in a subject comprising administering a therapeutically effective amount of the compound or salt of any one of claims 1 to 58 to the subject.

72. The method of claim 70 or 71, wherein the lysosomal protein is progranulin, prosaposin, β-glucocerebrosidase, galactosidase alpha, cathepsin B, cathepsin Z, neuraminidase 1, tripeptidyl peptidase, alpha-L-fucosidase 2, mannosidase alpha class 2B member 2, mannosidase beta, serine carboxypeptidase 1, acid ceramidase, GM2 ganglioside activator, cathepsin D, cathepsin S, cathepsin K, cathepsin L, or hexosaminidase.

73. A method of treating a lysosomal storage disorder in a subject suffering therefrom comprising administering a therapeutically effective amount of the compound or salt of any one of claims 1 to 58 to the subject.

74. The method of claim 73, wherein the lysosomal storage disorder is mucopolysaccharidosis, sphingolipidosis, glycogen storage disease type II, glycoprotein storage disease, Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, mucopolysaccharidosis-plus syndrome, Fabry disease, Gaucher disease, Tay-Sachs disease, sialidosis, Niemann Pick type A, Niemann Pick type B, galactosialidosis, Niemann pick type C, I-cell disease, mucolipidosis type III, GM1 gangliosidosis, μ-galactosidase deficiency, α-mannosidosis, GM2 gangliosidosis, β-mannosidosis, Krabbe, fucosidosis, metachromatic leukodystrophy, aspartylglucosaminuria, multiple sulfatase deficiency, Schindler, Farber lipogranulomatosis, Pompe disease, Wolman disease, Danon disease, free sialic acid storage disease, ceroid lipofuscinosis, β-glucuronidase hypoactivity disease, Sandhoff disease, or cholesterol ester storage disease.

75. A method of treating an inflammatory disorder in a subject suffering therefrom comprising administering a therapeutically effective amount of the compound or salt of any one of claims 1 to 58 to the subject.

76. The method of claim 75, wherein the inflammatory disorder is Sjogren disease, inflammatory arthritis, osteoarthritis, inflammatory bowel disease, or immune thrombocytopenia.

77. A method of treating a disorder in a subject suffering therefrom comprising administering a therapeutically effective amount of the compound or salt of any one of claims 1 to 58 to the subject, wherein the disorder is stroke, Down syndrome, congenital heart disease, diabetes, common variable immune deficiency (CVID), tubulo-interstitial kidney disease (TKD), polycystic liver disease, myocarditis, dermatitis hyperhomocysteinemia, endo-toxic shock, lung injury, bone defect (e.g., inflammatory periodontal bone defect), or osteolysis.

78. A method of treating a neurodegenerative disease in a subject suffering therefrom comprising administering a therapeutically effective amount of the compound or salt of any one of claims 1 to 58 to the subject.

79. The method of claim 78, wherein the neurodegenerative disease is Parkinson's disease, frontotemporal dementia, Alzheimer's disease, Huntington's disease, traumatic brain injury, neuronal ceroid lipofuscinosis (NCL), multiple sclerosis, amyotrophic lateral sclerosis (ALS), aigyrophilic grain dementia, Alexander's disease, Alper's disease, cerebral palsy, Cockayne syndrome, corticobasal degeneration, Creutzfeldt-Jakob disease, dementia pugilistica, diffuse neurofibrillary tangles with calcification, HIV-associated dementia, lewy body dementia, Kennedy's disease, neuroborreliosis, primary lateral sclerosis, Refsum's disease, Gerstmann-Straussler-Scheinker disease, Hallevorden-Spatz disease, hereditary diffuse leukoencepholopathy with spheroids (HDLS), inclusion body myositis, multiple system atrophy, myotonic dystrophy, Nasu-Hakola disease, Schilder's disease, Wobbly Hedgehog Syndrome (WHS), Duchenne-Aran muscular atrophy, progressive bulbar palsy, pseudobulbar palsy, HIV-associated neurocognitive disorder (HAND), tauopathy, chronic traumatic encephalopathy, or cerebellar downbeat nystagmus.
